(19) United States
(12) Patent Application Publication
(10) Pub. No.: US 2026/0166085 A1
(43) Pub. Date: Jun. 18, 2026

(54) COMPOSITIONS AND METHODS FOR NON-GENOTOXIC CELL CONDITIONING

(71) Applicant: Beam Therapeutics Inc., Cambridge, MA (US)

(72) Inventors: Nicole Gaudelli, Cambridge, MA (US); Tanggis Bohnuud, Cambridge, MA (US); Scott Haihua Chu, Cambridge, MA (US); Kathy Ying Zhang, Cambridge, MA (US); Nandini Mondal, Cambridge, MA (US); Megan Egbert, Cambridge, MA (US); Adam Hartigan, Cambridge, MA (US); Charlotte McDonagh, Cambridge, MA (US)

(73) Assignee: Beam Therapeutics Inc., Cambridge, MA (US)

(21) Appl. No.: 18/984,508

(22) Filed: Dec. 17, 2024

Related U.S. Application Data
(63) Continuation of application No. PCT/US2023/069189, filed on Jun. 27, 2023.
(60) Provisional application No. 63/355,927, filed on Jun. 27, 2022.
(60) Provisional application No. 63/386,719, filed on Dec. 9, 2022.
(60) Provisional application No. 63/478,744, filed on Jan. 6, 2023.
(60) Provisional application No. 63/500,854, filed on May 8, 2023.

(51) Int. Cl. A61K 35/28; A61K 39/00; A61P 37/06; C07K 16/28; C12N 5/0789; C12N 9/22; C12N 9/78; C12N 15/11

(52) U.S. Cl. CPC A61K 35/28; A61P 37/06; C07K 16/2803; C12N 5/0647; C12N 9/22; C12N 9/78; C12N 15/111; A61K 2039/505; C07K 2317/565; C07K 2317/92; C07K 2317/94; C12N 2310/20; C12N 2510/00; C12Y 305/04004

(57) ABSTRACT

Compositions and methods for non-genotoxic monoclonal antibody (mAb) conditioning, where the methods involve altering a cluster of differentiation 117 (CD117; c-KIT) polynucleotide sequence in a hematopoietic stem cell (HSC) or progenitor thereof to encode a CD117 polypeptide with reduced binding to the antibody. In various embodiments, the methods further include introducing a therapeutic alteration to a gene of the HSC or progenitor thereof for treatment of a hemoglobinopathy.

### CROSS REFERENCE TO RELATED APPLICATIONS

This application is a continuation under 35 U.S.C. § 111 (a) of PCT International Patent Application No. PCT/US2023/069189, filed Jun. 27, 2023, which claims priority to and the benefit of U.S. Provisional Application No. 63/500,854, filed May 8, 2023, U.S. Provisional Application No. 63/478,744, filed Jan. 6, 2023, U.S. Provisional Application No. 63/386,719, filed Dec. 9, 2022, and U.S. Provisional Application No. 63/355,927, filed Jun. 27, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Jun. 26, 2023, is named 180802-046405PCT_SL.xml and is 1,559,644 bytes in size.

### BACKGROUND

Busulfan is a DNA alkylating reagent that induces bone marrow immunosuppression and is widely used for conditioning prior to allogenic hematopoietic stem cell transplantation and administration of autologous cell therapies. Notably, a prerequisite for ex vivo treatment of sickle cell disease (SCD), the most common single gene inherited hemoglobinopathy, is conditioning of the patient prior to infusion of an autologous cell therapy. While busulfan is the current standard of care for patients in need of allogenic or autologous transplants and engraftment of cell therapies, the use of this potent cytotoxic agent has associated risks including genotoxicity, primary or secondary malignancy, and organ toxicities including infertility. These risks present barriers to patients who would otherwise seek treatment. Accordingly, there is a need for improved methods for conditioning prior to allogeneic hematopoietic stem cell transplantation.

### SUMMARY

As described below, the present disclosure features compositions and methods for non-genotoxic monoclonal antibody (mAb) conditioning, where the methods involve altering a cluster of differentiation 117 (CD117; c-KIT) polynucleotide sequence in a hematopoietic stem cell (HSC) or progenitor thereof to encode a CD117 polypeptide with reduced binding to the antibody. In various embodiments, the methods further include introducing a therapeutic alteration to a gene of the HSC or progenitor thereof for treatment of a hemoglobinopathy (e.g., sickle cell disease).

In one aspect, the disclosure features a method of altering a nucleobase of a CD117 polynucleotide. The method involves, contacting the CD117 polynucleotide with a base editor polypeptide containing a nucleic acid programmable DNA binding protein (napDNAbp) and an adenosine deaminase domain, and a guide polynucleotide that targets the base editor to i) effect an alteration of a nucleobase in a codon encoding a serine at amino acid position 261, where the alteration of the nucleobase encoding the serine at amino acid position 261 results in the codon expressing a glycine, ii) effect an alteration of a nucleobase in a codon encoding a serine at amino acid position 251, and/or iii) effect an alteration of a nucleobase in a codon encoding an asparagine at amino acid position 260 and effect an alteration of a nucleobase in a codon encoding a serine at amino acid position 261, or corresponding positions in another CD117 polypeptide, thereby altering the nucleobase of the CD117 polynucleotide.

In another aspect, the disclosure features a method of altering a nucleobase of a CD117 polynucleotide, the method involves contacting the CD117 polynucleotide with a base editor polypeptide containing a nucleic acid programmable DNA binding protein (napDNAbp) domain and an adenosine deaminase domain. The adenosine deaminase domain contains a combination of alterations to TadA*7.10 selected from: a) I76Y, V82T, Y123H, Y147R, F149Y, and Q154R; and b) I76Y, V82T, Y123H, Y147D, F149Y, Q154R, T166I, and D167N. The adenosine deaminase domain has at least 85% sequence identity to TadA*7.10. The method also involves contacting the CD117 polynucleotide with a guide polynucleotide that targets the base editor to effect an alteration of a nucleobase in a polynucleotide encoding a CD117 polypeptide, thereby altering the nucleobase of the CD117 polynucleotide.

In another aspect, the disclosure features a method for hematopoietic stem cell transplantation in a subject. The method involves (a) contacting an isolated hematopoietic stem cell or progenitor thereof with a guide polynucleotide and a base editor containing a nucleic acid programmable DNA binding protein (napDNAbp) and an adenosine deaminase domain or a polynucleotide encoding the base editor, where the guide polynucleotide targets a nucleic acid molecule encoding a CD117 polypeptide. (a) results in the generation of an edited cell. The method also involves (b) administering the edited cell to the subject. The method also involves (c) administering to the subject an antibody or antigen binding fragment thereof, where the antibody is selected from one or more of ABTx025, ABTx030, ABTx052, ABTx061, ABTx062, ABTx070, ABTx071, ABTx196, ABTx198, ABTx202, ABTx203, ABTx205, ABTx206, ABTx248, ABTx250, ABTx251, ABTx253, ABTx254, ABTx255, ABTx256, ABTx265, ABTx268, ABTx270, ABTx271, ABTx272, ABTx273, ABTx274, ABTx307, ABTx308, ABTx309, and ABTx313.

In another aspect, the disclosure features a method for hematopoietic stem cell transplantation in a subject. The method involves (a) contacting an isolated hematopoietic stem cell or progenitor thereof with a guide polynucleotide and a base editor containing a nucleic acid programmable DNA binding protein (napDNAbp) and an adenosine deaminase domain or a polynucleotide encoding the base editor. The guide polynucleotide targets a nucleic acid molecule encoding a CD117 polypeptide, thereby i) introducing an alteration of a nucleobase in a codon encoding a serine at amino acid position 261, where the alteration of the nucleobase encoding the serine at amino acid position 261 results in the codon expressing a glycine, ii) introducing an alteration of a nucleobase in a codon encoding a serine at amino acid position 251, and/or iii) introducing an alteration of a nucleobase in a codon encoding an asparagine at amino acid position 260 and introducing an alteration of a nucleobase in a codon encoding a serine at amino acid position 261, or corresponding positions in another CD117 polypeptide. (a) results in generating an edited cell. The method also involves (b) administering the edited cell to the subject. The method also involves (c) administering to the subject an antibody or antigen binding fragment thereof, antibody drug conjugate, or a chimeric antigen receptor T (CAR-T) cell, each of which selectively binds a wild type CD117 polypeptide.

In another aspect, the disclosure features a method for hematopoietic stem cell transplantation in a subject. The method involves (a) contacting an isolated hematopoietic stem cell or progenitor thereof with a guide polynucleotide and a base editor containing a nucleic acid programmable DNA binding protein (napDNAbp) and an adenosine deaminase domain or a polynucleotide encoding the base editor. The adenosine deaminase domain contains a combination of alterations to TadA*7.10 selected from: i) I76Y, V82T, Y123H, Y147R, F149Y, and Q154R; and ii) I76Y, V82T, Y123H, Y147D, F149Y, Q154R, T166I, and D167N. The adenosine deaminase domain has at least 85% sequence identity to TadA*7.10. The guide polynucleotide targets a nucleic acid molecule encoding a CD117 polypeptide. (a) results in generating an edited cell. The method also involves (b) administering the edited cell to the subject. The method also involves (c) administering to the subject an antibody or antigen binding fragment thereof, antibody drug conjugate, or a chimeric antigen receptor T (CAR-T) cell, each of which selectively binds a wild type CD117 polypeptide.

In another aspect, the disclosure features a method for treating a hemoglobinopathy in a subject. The method involves (a) contacting an isolated hematopoietic stem cell or progenitor thereof with two or more guide polynucleotides and a base editor containing a nucleic acid programmable DNA binding protein (napDNAbp) and an adenosine deaminase domain or a polynucleotide encoding the base editor. One guide polynucleotide targets a nucleic acid molecule encoding a CD117 polypeptide and another guide polynucleotide targets the base editor to effect a deamination of a nucleobase of a hemoglobin subunit gamma 1 and/or 2 (HBG1/2) promoter. (a) results in generating an edited cell. The method also involves (b) administering the edited cell to the subject. The method also involves (c) administering to the subject an antibody or antigen binding fragment thereof, where the antibody is selected from one or more of ABTx025, ABTx030, ABTx052, ABTx061, ABTx062, ABTx070, ABTx071, ABTx196, ABTx198, ABTx202, ABTx203, ABTx205, ABTx206, ABTx248, ABTx250, ABTx251, ABTx253, ABTx254, ABTx255, ABTx256, ABTx265, ABTx268, ABTx270, ABTx271, ABTx272, ABTx273, ABTx274, ABTx307, ABTx308, ABTx309, and ABTx313.

In another aspect, the disclosure features a method for treating a hemoglobinopathy in a subject. The method involves (a) contacting an isolated hematopoietic stem cell or progenitor thereof with two or more guide polynucleotides and a base editor containing a nucleic acid programmable DNA binding protein (napDNAbp) and an adenosine deaminase domain or a polynucleotide encoding the base editor. One guide polynucleotide targets a nucleic acid molecule encoding a CD117 polypeptide, thereby i) introducing an alteration of a nucleobase in a codon encoding a serine at amino acid position 261, where the alteration of the nucleobase encoding the serine at amino acid position 261 results in the codon expressing a glycine, ii) introducing an alteration of a nucleobase in a codon encoding a serine at amino acid position 251, and/or iii) introducing an alteration of a nucleobase in a codon encoding an asparagine at amino acid position 260 and introducing an alteration of a nucleobase in a codon encoding a serine at amino acid position 261, or corresponding positions in another CD117 polypeptide. Another guide polynucleotide targets the base editor to effect an alteration to a beta globin polynucleotide (HBB) that results in expression of a beta globin polypeptide having an alanine at position 6 (Hb G-Makassar). (a) also results in generating an edited cell. The method also involves (b) administering the edited cell to the subject. The method further involves (c) administering to the subject an antibody or antigen binding fragment thereof, antibody drug conjugate, or a chimeric antigen receptor T (CAR-T) cell, each of which selectively binds a wild type CD117 polypeptide.

In another aspect, the disclosure features a method for treating a hemoglobinopathy in a subject. The method involves (a) contacting an isolated hematopoietic stem cell or progenitor thereof with two or more guide polynucleotides and a base editor containing a nucleic acid programmable DNA binding protein (napDNAbp) and an adenosine deaminase domain or a polynucleotide encoding the base editor. The adenosine deaminase domain contains a combination of alterations to TadA*7.10 selected from: i) 176Y, V82T, Y123H, Y147R, F149Y, and Q154R; and ii) I76Y, V82T, Y123H, Y147D, F149Y, Q154R, T166I, and D167N. The adenosine deaminase domain has at least 85% sequence identity to TadA*7.10. One guide polynucleotide targets a nucleic acid molecule encoding a CD117 polypeptide, and another guide polynucleotide targets the base editor to effect an alteration to a beta globin polynucleotide (HBB) that results in expression of a beta globin polypeptide having an alanine at position 6 (Hb G-Makassar). (a) results in generating an edited cell. The method also involves (b) administering the edited cell to the subject. The method also involves (c) administering to the subject an antibody or antigen binding fragment thereof, antibody drug conjugate, or a chimeric antigen receptor T (CAR-T) cell, each of which selectively binds a wild type CD117 polypeptide.

In another aspect, the disclosure features a cell produced by the method of any of the above aspects, or embodiments thereof.

In another aspect, the disclosure features a pharmaceutical composition containing an effective amount of the cell of any of the above aspects, or embodiments thereof.

In another aspect, the disclosure features a base editor system containing a base editor containing a nucleic acid programmable DNA binding protein (napDNAbp) and an adenosine deaminase domain or a polynucleotide encoding the base editor, and a guide polynucleotide that targets the base editor to i) effect an alteration of a nucleobase in a codon encoding a serine at amino acid position 261, where the alteration of the nucleobase encoding the serine at amino acid position 261 results in the codon expressing a glycine, ii) effect an alteration of a nucleobase in a codon encoding a serine at amino acid position 251, and/or iii) effect an alteration of a nucleobase in a codon encoding an asparagine at amino acid position 260 and effect an alteration of a nucleobase in a codon encoding a serine at amino acid position 261, or corresponding positions in another CD117 polypeptide, thereby altering the nucleobase of the CD117 polynucleotide.

In another aspect, the disclosure features a base editor system containing a guide polynucleotide and a base editor containing a nucleic acid programmable DNA binding protein (napDNAbp) domain and an adenosine deaminase domain. The adenosine deaminase domain contains a combination of alterations to TadA*7.10. The combinations are selected from: a) I76Y, V82T, Y123H, Y147R, F149Y, and Q154R; and b) I76Y, V82T, Y123H, Y147D, F149Y, Q154R, T166I, and D167N. The guide polynucleotide targets the base editor to effect an alteration of a nucleobase of a CD117 polynucleotide. The adenosine deaminase domain has at least 85% sequence identity to TadA*7.10.

In another aspect, the disclosure features a base editor system containing a base editor containing a nucleic acid programmable DNA binding protein (napDNAbp) and an adenosine deaminase domain, and a guide polynucleotide containing a polynucleotide sequence selected from one or more of: AUAAUAGCUGGCAUCACGGU (SEQ ID NO: 693; gRNA931); CCACUAGCUUUCCAAACGGU (SEQ ID NO: 694; gRNA889); GCUGAACUGAUAGUCAACGU (SEQ ID NO: 695; gRNA908); UUUGACAAAGCCCGGAUCAG (SEQ ID NO: 696; gRNA918); UGAAAGUGAGGCCAGGUACU (SEQ ID NO: 697; gRNA923); AAACAGUCAGGUGAGUGAAU (SEQ ID NO: 698; gRNA928); AACUACAGGAGAAAUAUAAU (SEQ ID NO: 699; gRNA929); and GAUUAAAAGGCACCGAAGGA (SEQ ID NO: 700; gRNA944).

In another aspect, the disclosure features a polynucleotide encoding the base editor system of any of the above aspects, or embodiments thereof.

In another aspect, the disclosure features a guide polynucleotide containing a spacer sequence selected from one or more of: AUAAUAGCUGGCAUCACGGU (SEQ ID NO: 693; gRNA931); CCACUAGCUUUCCAAACGGU (SEQ ID NO: 694; gRNA889); GCUGAACUGAUAGUCAACGU (SEQ ID NO: 695; gRNA908); UUUGACAAAGCCCGGAUCAG (SEQ ID NO: 696; gRNA918); UGAAAGUGAGGCCAGGUACU (SEQ ID NO: 697; gRNA923); AAACAGUCAGGUGAGUGAAU (SEQ ID NO: 698; gRNA928); AACUACAGGAGAAAUAUAAU (SEQ ID NO: 699; gRNA929); and GAUUAAAAGGCACCGAAGGA (SEQ ID NO: 700; gRNA944).

In another aspect, the disclosure features a kit containing the cell, base editor system, polynucleotide, or pharmaceutical composition of any of the above aspects, or embodiments thereof.

In another aspect, the disclosure features an anti-CD117 antibody or antigen-binding portion thereof containing one or more complementarity determining regions (CDRs) which contain heavy chain variable region (VH) CDRs and/or light chain variable region (VL) CDRs selected from the following: A) VL CDR1: QSVSSSY (SEQ ID NO: 394); VL CDR2: GAS; VL CDR3: QQYGTSLT (SEQ ID NO: 395); VH CDR1: GFTFDDYA (SEQ ID NO: 391); VH CDR2: ISWNSGTI (SEQ ID NO: 392); VH CDR3: AKDSPPGYCSSASCYGAFDI (SEQ ID NO: 393) (ABTx025); B) VL CDR1: QSISSY (SEQ ID NO: 409); VL CDR2: AAS; VL CDR3: QQSYSTPLT (SEQ ID NO: 410); VH CDR1: GFTFSSYS (SEQ ID NO: 406); VH CDR2: IGTISSYI (SEQ ID NO: 407); VH CDR3: ARDYYGGLFDY (SEQ ID NO: 408) (ABTx030); C) VL CDR1: QSVSSSY (SEQ ID NO: 439); VL CDR2: GAS; VL CDR3: QQYGSSPLT (SEQ ID NO: 440); VH CDR1: GFTFDDYA (SEQ ID NO: 436); VH CDR2: ISWNSGSI (SEQ ID NO: 437); VH CDR3: AKDTPLGYCSTTSCYGAFDI (SEQ ID NO: 438) (ABTx061); D) VL CDR1: QSISSY (SEQ ID NO: 454); VL CDR2: AAS; VL CDR3: QQSYSTPFT (SEQ ID NO: 455); VH CDR1: GFTFDDYA (SEQ ID NO: 451); VH CDR2: ISWNSGTI (SEQ ID NO: 452); VH CDR3: AKDSPPGYCSSASCYGAFDI (SEQ ID NO: 453) (ABTx062); E) VL CDR1: QGISSY (SEQ ID NO: 469); VL CDR2: AAS; VL CDR3: QQSYSTPIT (SEQ ID NO: 470); VH CDR1: GFTEDDYG (SEQ ID NO: 466); VH CDR2: INWNGGST (SEQ ID NO: 467); VH CDR3: ARESWDGSGIYYMDV (SEQ ID NO: 468) (ABTx070); F) VL CDR1: QGISSY (SEQ ID NO: 484); VL CDR2: AAS; VL CDR3: QQLNSYPYT (SEQ ID NO: 485); VH CDR1: GFTEDDYG (SEQ ID NO: 481); VH CDR2: INWNGGST (SEQ ID NO: 482); VH CDR3: ARESWNYEGYYYMDV (SEQ ID NO: 483) (ABTx071); G) VL CDR1: QGISSA (SEQ ID NO: 424); VL CDR2: DAS; VL CDR3: QQFNSYPLT (SEQ ID NO: 425); VH CDR1: GYRFTSYW (SEQ ID NO: 421); VH CDR2: IYPGDSDTR (SEQ ID NO: 958); VH CDR3: ARHGRGYDGYDGAFDI (SEQ ID NO: 1012); H) VL CDR1: QGISSA (SEQ ID NO: 424); VL CDR2: DAS; VL CDR3: QQFNSYPLT (SEQ ID NO: 425); VH CDR1: GYRFTSYW (SEQ ID NO: 421); VH CDR2: IYPGDSDTK (SEQ ID NO: 972); VH CDR3: ARHGRGYDGYEGAFDI (SEQ ID NO: 948); I) VL CDR1: QGISSA (SEQ ID NO: 424); VL CDR2: DAS; VL CDR3: QQFYSYPLT (SEQ ID NO: 954); VH CDR1: GYRFTSYW (SEQ ID NO: 421); VH CDR2: TYPGDSDTR (SEQ ID NO: 958); VH CDR3: ARHGRGYDGYEGAFDI (SEQ ID NO: 948); J) VL CDR1: QGISSA (SEQ ID NO: 424); VL CDR2: DAS; VL CDR3: QQFSSYPLT (SEQ ID NO: 1020); VH CDR1: GYRFTSYW (SEQ ID NO: 421); VH CDR2: IYPGDSDTR (SEQ ID NO: 958); VH CDR3: ARHGRGYDGYEGAFDI (SEQ ID NO: 948); K) VL CDR1: QGISSA (SEQ ID NO: 424); VL CDR2: DAS; VL CDR3: QQTNSHPLT (SEQ ID NO: 1023); VH CDR1: GYRFTSYW (SEQ ID NO: 421); VH CDR2: IYPGDSDTK (SEQ ID NO: 972); VH CDR3: ARHGRGYDGYDGAFDI (SEQ ID NO: 1012); L) VL CDR1: QGISSA (SEQ ID NO: 424); VL CDR2: DAS; VL CDR3: QQVRSYPLT (SEQ ID NO: 1025); VH CDR1: GYRFTSYW (SEQ ID NO: 421); VH CDR2: IYPGDSDTK (SEQ ID NO: 972); VH CDR3: ARHGRGYDGYEGAFDI (SEQ ID NO: 948); M) VL CDR1: QGISSA (SEQ ID NO: 424); VL CDR2: DAS; VL CDR3: QQTYSYPLT (SEQ ID NO: 1029); VH CDR1: GYRFTSYW (SEQ ID NO: 421); VH CDR2: IYPGDSDTR (SEQ ID NO: 958); VH CDR3: ARHGRGYDAYEGAFDI (SEQ ID NO: 1027); N) VL CDR1: QGISSA (SEQ ID NO: 424); VL CDR2: DAS; VL CDR3: QQTRSYPLT (SEQ ID NO: 1036); VH CDR1: GYRFTSYW (SEQ ID NO: 421); VH CDR2: TYPGDSDTK (SEQ ID NO: 972); VH CDR3: ARHGRGYDGYDGAFDI (SEQ ID NO: 1012); O) VL CDR1: QGISSA (SEQ ID NO: 424); VL CDR2: DAS; VL CDR3: QQVNSYPLT (SEQ ID NO: 952); VH CDR1: GYRFTSYW (SEQ ID NO: 421); VH CDR2: TYPGDSDTK (SEQ ID NO: 972); VH CDR3: ARHGRGYDGYEGAFDI (SEQ ID NO: 948); P) VL CDR1: QGISSA (SEQ ID NO: 424); VL CDR2: DAS; VL CDR3: QQVRSYPLT (SEQ ID NO: 1025); VH CDR1: GYRFTSYW (SEQ ID NO: 421); VH CDR2: IYPGDSDTR (SEQ ID NO: 958); VH CDR3: ARHGRGYDAYEGAFDI (SEQ ID NO: 1027); Q) VL CDR1: QGISSA (SEQ ID NO: 424); VL CDR2: DAS; VL CDR3: QQTNSYPLT (SEQ ID NO: 1044); VH CDR1: GYRFTSYW (SEQ ID NO: 421); VH CDR2: IYPGDSDTR (SEQ ID NO: 958); VH CDR3: ARHGRGYDAYDGAFDI (SEQ ID NO: 1032); R) VL CDR1: QGISSA (SEQ ID NO: 424); VL CDR2: DAS; VL CDR3: QQVRSYPLT (SEQ ID NO: 1025); VH CDR1: GYRFTSYW (SEQ ID NO: 421); VH CDR2: TYPGDSDTK (SEQ ID NO: 972); VH CDR3: ARHGRGYDGYDGAFDI (SEQ ID NO: 1012); S) VL CDR1: QGISSA (SEQ ID NO: 424); VL CDR2: DAS; VL CDR3: QQFRSYPLT (SEQ ID NO: 953); VH CDR1: GYRFTSYW (SEQ ID NO: 421); VH CDR2: TYPGDSDTR (SEQ ID NO: 958); VH CDR3: ARHGRGYDGYEGAFDI (SEQ ID NO: 948); T) VL CDR1: QGISSA (SEQ ID NO: 424); VL CDR2: DAS; VL CDR3: QQTNSYPLT (SEQ ID NO: 1044); VH CDR1: GYRFTSYW (SEQ ID NO: 421); VH CDR2: IYPGDSDTK (SEQ ID NO: 972); VH CDR3: ARHGRGYDGYEGAFDI (SEQ ID NO: 948); U) VL CDR1: QSVSSSY (SEQ ID NO: 394); VL CDR2: GAS; VL CDR3: QQSETCLT (SEQ ID NO: 1074); VH CDR1: GFTEDDYA (SEQ ID NO: 391) VH CDR2: ISWNSGTIG (SEQ ID NO: 1070); VH CDR3: AKDSPPGYCSSASCYGAFDI (SEQ ID NO: 393); V) VL CDR1: QSVSSSY (SEQ ID NO: 394); VL CDR2: GAS; VL CDR3: QQDSLGLT (SEQ ID NO: 1074); VH CDR1: GFTFDDYA (SEQ ID NO: 391); VH CDR2: ISWNSGTIG (SEQ ID NO: 1070); VH CDR3: AKDSPPGYCASASCYGAFDI (SEQ ID NO: 1090); W) VL CDR1: QSVSSSY (SEQ ID NO: 394); VL CDR2: GSS; VL CDR3: QQYNFWPYT (SEQ ID NO: 1084); VH CDR1: GFTFDDYA (SEQ ID NO: 391); VH CDR2: ISWNSGTIG (SEQ ID NO: 1070); VH CDR3: AKDSPPGYCSSASCYGAFDI (SEQ ID NO: 393); X) VL CDR1: QGISSY (SEQ ID NO: 469); VL CDR2: AAS; VL CDR3: QQSYSTPYT (SEQ ID NO: 1097); VH CDR1: GFTFDDYA (SEQ ID NO: 391); VH CDR2: ISWNSGTIG (SEQ ID NO: 1070); VH CDR3: AKDWPSGFCSSAYCYGAFDI (SEQ ID NO: 1094); Y) VL CDR1: QGISSY (SEQ ID NO: 469); VL CDR2: AAS; VL CDR3: QQSYSTPYT (SEQ ID NO: 1097); VH CDR1: GFTFDDYA (SEQ ID NO: 1071); VH CDR2: ISWNSGTIG (SEQ ID NO: 1070); VH CDR3: AKDSPPGYCSSASCYGAFDI (SEQ ID NO: 393); Z) VL CDR1: QGISSA (SEQ ID NO: 424); VL CDR2: DAS; VL CDR3: QQFNSYPLT (SEQ ID NO: 425); VH CDR1: GYRFTSYW (SEQ ID NO: 421); VH CDR2: TYPGDSDTR (SEQ ID NO: 958); VH CDR3: ARHGRGYDGYDGAFDI (SEQ ID NO: 1012); AA) VL CDR1: QGISSA (SEQ ID NO: 424); VL CDR2: DAS; VL CDR3: QQFSSYPLT (SEQ ID NO: 1020); VH CDR1: GYRFTSYW (SEQ ID NO: 421); VH CDR2: IYPGDSDTK (SEQ ID NO: 972); VH CDR3: ARHGRGYDAYDGAFDI (SEQ ID NO: 1032); AB) VL CDR1: QGISSA (SEQ ID NO: 424); VL CDR2: DAS; VL CDR3: QQTNSYPLT (SEQ ID NO: 1044); VH CDR1: GYRFTSYW (SEQ ID NO: 421); VH CDR2: IYPGDSDTR (SEQ ID NO: 958); VH CDR3: ARHGRGYDAYDGAFDI (SEQ ID NO: 1032); and AC) VL CDR1: QGISSA (SEQ ID NO: 424); VL CDR2: DAS; VL CDR3: QQFSSYPLT (SEQ ID NO: 425); VH CDR1: GYRFTSYW (SEQ ID NO: 421); VH CDR2: IYPGDSDTR (SEQ ID NO: 958); VH CDR3: ARHGRGYDGYDGAFDI (SEQ ID NO: 1012).

In another aspect, the disclosure features an isolated nucleic acid molecule that encodes the antibody of any one of any of the above aspects, or embodiments thereof.

In another aspect, the disclosure features an anti-CD117 antibody or antigen-binding portion thereof containing complementarity determining regions (CDRs) that contain the following heavy chain variable region (VH) CDR and light chain variable region (VL) CDR amino acid sequences: VH CDR1: GYRFTSYW (SEQ ID NO: 421); VH CDR2 is IYPGDSDTR (SEQ ID NO: 958) or TYPGDSDTK (SEQ ID NO: 972); VH CDR3: ARHGRGYXYXGAFDI (SEQ ID NO: 944); VL CDR1: QGISSA (SEQ ID NO: 424); VL CDR2: DAS; and VL CDR3 selected from one or more of QQXSYPLT (SEQ ID NO: 945), QQXSHPLT (SEQ ID NO: 946), and QQXSSPLT (SEQ ID NO: 947). X indicates any amino acid. The anti-CD117 antibody contains at least one amino acid alteration relative to the amino acid sequence of ABTx052.

In another aspect, the disclosure features An anti-CD117 antibody or antigen-binding portion thereof containing complementarity determining regions (CDRs) that contain or contain only the following heavy chain variable region (VH) CDR and light chain variable region (VL) CDR amino acid sequences:
VH CDR1: GX₁X₂FX₃X₄YX₅, where X₁ is F or Y, X₂ is R or T, X₃ is D, S, or T, X₄ is D or S, and X₅ is A, G, S, or W;
VH CDR2 is IX₆X₇X₈X₉X₁₀X₁₁X₁₂X₁₃, where X₆ is G, N, S, or Y, X₇ is P, T, or W, X₈ is G, I, or N, X₉ is D, G, or S, X₁₀ is G or S, X₁₁ is D, S, T, or Y, X₁₂ is I or T, and X₁₃ is G, K, R, or Y; VH CDR3 is selected from one or more of ARHGRGYDX₁₄ YDGAFDI (SEQ ID NO: 1105), ARDYYGGLFDY (SEQ ID NO: 1106), ARESWX₁₅X₁₆X₁₇GX₁₈ YYMDV (SEQ ID NO: 1107), and AKDX₁₉PX₂₀GX₂₁CX₂₂X₂₃X₂₄X₂₅CYGAFDI (SEQ ID NO: 1108), where X₁₄ is A or G, X₁₅ is D or N, X₁₆ is G or Y, X₁₇ is E or S, X₁₈ is I or Y, X₁₉ is S, T, or W, X₂₀ is L, P, or S, X₂₁ is F or Y, X₂₂ is A or S, X₂₃ is S or T, X₂₄ is A or T, and X₂₅ is S or Y;
VL CDR1 is QSX₂₆SSX₂₇ (SEQ ID NO: 1109) or QSVSSSY (SEQ ID NO: 1110), where X₂₆ is G or S, and X₂₇ is A or Y;
VL CDR2: X₂₈X₂₉S, where X₂₈ is A, D, or G, and X₂₉ is A or S; and
VL CDR3 is QQX₃₀X₃₁X₃₂X₃₃ PX₃₄T (SEQ ID NO: 1111) or QQX₃₅X₃₆X₃₇X₃₈LT (SEQ ID NO: 1112), where X₃₀ is F, L, S, T, or Y, X₃₁ G, N, S, or Y, X₃₂ is S or F, X₃₃ S, T, W, or Y, X₃₄ F, I, L, or Y, X₃₅ is D, S, or Y, X₃₆ is E, G, or S, X₃₇ is L or T, and X₃₈ is C, G, or S. The anti-CD117 antibody contains at least one amino acid alteration relative to the amino acid sequence of ABTx052.

In another aspect, the disclosure features a method for hematopoietic stem cell transplantation in a subject. The method involves (a) administering a hematopoietic stem cell or progenitor thereof to the subject. The hematopoietic stem cell or progenitor thereof expresses a CD117 variant containing an S261G amino acid alteration, or a CD117 variant containing Y259C and N260D amino acid alterations. The method further involves (b) administering to the subject an antibody or antigen binding fragment thereof that selectively binds a wild type CD117 polypeptide.

In another aspect, the disclosure features a method for treating a hemoglobinopathy in a subject. The method involves (a) administering a hematopoietic stem cell or progenitor thereof to the subject. The hematopoietic stem cell or progenitor thereof: i) either expresses a CD117 variant containing an S261G amino acid alteration or a CD117 variant containing Y259C and N260D amino acid alterations, and ii) contains a nucleobase alteration to the HBG1/2 promoter that effects an increase in gamma globin expression and/or expresses an HBB polypeptide containing an alanine at position 6. The method further involves, (b) administering to the subject an antibody or antigen binding fragment thereof that selectively binds a wild type CD117 polypeptide.

In another aspect, the disclosure features a hematopoietic stem cell or progenitor thereof expressing i) an S261G alteration, ii) alterations at amino acid positions 260 and 261, and/or ii) an alteration at amino acid position 251 relative to the following amino acid sequence, where the CD117 polypeptide has at least 85% sequence identity to the following amino acid sequence:

Wild Type CD117
(SEQ ID NO: 499)
MRGARGAWDFLCVLLLLLRVQTGSSQPSVSPGEPSPPSIHPGKSDLIVRVGDEIRLLCTDPGFV
KWTFEILDETNENKQNEWITEKAEATNTGKYTCTNKHGLSNSIYVFVRDPAKLFLVDRSLYGKE
DNDTLVRCPLTDPEVTNYSLKGCQGKPLPKDLRFIPDPKAGIMIKSVKRAYHRLCLHCSVDQEG
KSVLSEKFILKVRPAFKAVPVVSVSKASYLLREGEEFTVTCTIKDVSSSVYSTWKRENSQTKLQ
EKYNSWHHGDFNYERQATLTISSARVNDSGVFMCYANNTFGSANVTTTLEVVDKGFINIFPMIN
TTVFVNDGENVDLIVEYEAFPKPEHQQWIYMNRTFTDKWEDYPKSENESNIRYVSELHLTRLKG
TEGGTYTFLVSNSDVNAAIAFNVYVNTKPEILTYDRLVNGMLQCVAAGFPEPTIDWYFCPGTEQ
RCSASVLPVDVQTLNSSGPPFGKLVVQSSIDSSAFKHNGTVECKAYNDVGKTSAYFNFAFKGNN
KEQIHPHTLFTPLLIGFVIVAGMMCIIVMILTYKYLQKPMYEVQWKVVEEINGNNYVYIDPTQL
PYDHKWEFPRNRLSFGKTLGAGAFGKVVEATAYGLIKSDAAMTVAVKMLKPSAHLTEREALMSE
LKVLSYLGNHMNIVNLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTPAIMEDDELALDLED
LLSFSYQVAKGMAFLASKNCIHRDLAARNILLTHGRITKICDFGLARDIKNDSNYVVKGNARLP
VKWMAPESIFNCVYTFESDVWSYGIFLWELFSLGSSPYPGMPVDSKFYKMIKEGFRMLSPEHAP
AEMYDIMKTCWDADPLKRPTFKQIVQLIEKQISESTNHIYSNLANCSPNRQKPVVDHSVRINSV
GSTASSSQPLLVHDDV.

In another aspect, the disclosure features a hematopoietic stem cell or progenitor thereof expressing a CD117 polypeptide containing a sequence containing 10, 20, 30, or 40 consecutive amino acids. The sequence of consecutive amino acids contains amino acid position 260, amino acid positions 261, and/or amino acid position 251 relative to the following wild type CD117 amino acid sequence. Also, i) an amino acid corresponding to amino acid position 261 is substituted with a glycine, ii) the amino acids corresponding to amino acid positions 260 and 261 are altered, and/or
iii) an amino acid corresponding to amino acid position 251 is altered relative to the following wild type CD117 amino acid sequence in the sequence of consecutive amino acids. The sequence of consecutive amino acids has at least 85% sequence identity to a fragment of the following wild type CD117 amino acid sequence that has the same length as the sequence of consecutive amino acids.
Wild Type CD117 amino acid sequence MRGARGAWDFLCVLLLLLRVQTGSSQPSVSPGEPSPPSIHPGKSDLIVRVGDEIRLLCTDPGFV KWTFEILDETNENKQNEWITEKAEATNTGKYTCTNKHGLSNSIYVFVRDPAKLFLVDRSLYGKE DNDTLVRCPLTDPEVTNYSLKGCQGKPLPKDLRFIPDPKAGIMIKSVKRAYHRLCLHCSVDQEG KSVLSEKFILKVRPAFKAVPVVSVSKASYLLREGEEFTVTCTIKDVSSSVYSTWKRENSQTKLQ EKYNSWHHGDFNYERQATLTISSARVNDSGVFMCYANNTFGSANVTTTLEVVDKGFINIFPMIN TTVFVNDGENVDLIVEYEAFPKPEHQQWI YMNRTFTDKWEDYPKSENESNIRYVSELHLTRLKG TEGGTYTFLVSNSDVNAAIAFNVYVNTKPEILTYDRLVNGMLQCVAAGFPEPTIDWYFCPGTEQ RCSASVLPVDVQTLNSSGPPFGKLVVQSSIDSSAFKHNGTVECKAYNDVGKTSAYFNFAFKGNN KEQIHPHTLFTPLLIGFVIVAGMMCIIVMILTYKYLQKPMYEVQWKVVEEINGNNYVYIDPTQL PYDHKWE FPRNRLSFGKTLGAGAFGKVVEATAYGLIKSDAAMTVAVKMLKPSAHLTEREALMSE LKVLSYLGNHMNIVNLLGACTIGGPTLVITEYCCYGDLLNFLRRKRDSFICSKQEDHAEAALYK NLLHSKESSCSDSTNEYMDMKPGVSYVVPTKADKRRSVRIGSYIERDVTPAIMEDDELALDLED LLSFSYQVAKGMAFLASKNCIHRDLAARNILLTHGRITKICDFGLARDI KNDSNYVVKGNARLP VKWMAPESI FNCVYTFESDVWSYGIFLWELFSLGSSPYPGMPVDSKFYKMI KEGFRMLSPEHAP AEMYDIMKTCWDADPLKRPTFKQIVQLIEKQISESTNHIYSNLANCSPNRQKPVVDHSVRINSV GSTASSSQPLLVHDDV (SEQ ID NO: 499). The CD117 polypeptide is capable of binding a stem cell factor (SCF) polypeptide.

In any aspect of the disclosure, or embodiments thereof, the adenosine deaminase is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In any aspect of the disclosure, or embodiments thereof, the adenosine deaminase domain contains a set of alterations to TadA*7.10: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1). The alterations are selected from the following: a) I76Y, V82T, Y123H, Y147R, F149Y, and Q154R (ABE9v1); b) I76Y, V82T, Y123H, Y147D, F149Y, Q154R, T166I, and D167N (ABE9v1); and c) I76Y, V82S, Y123H, Y147D, F149Y, Q154R, T166I, and D167N (ABE8.20+). The adenosine deaminase domain has at least 85% sequence identity to TadA*7.10.

In any aspect of the disclosure, or embodiments thereof, the deaminase is a monomer or heterodimer. In any aspect of the disclosure, or embodiments thereof, the base editor polypeptide is an internal base editor (IBE) containing the deaminase domain inserted at an internal location of the napDNAbp.

In any aspect of the disclosure, or embodiments thereof, the base editor polypeptide further contains one or more nuclear localization sequences (NLS). In any aspect of the disclosure, or embodiments thereof, the base editor polypeptide further contains a bipartite nuclear localization sequence (NLS).

In any aspect of the disclosure, or embodiments thereof, the deaminase domain is fused to the napDNAbp. In any aspect of the disclosure, or embodiments thereof, the napDNAbp is a nuclease inactive or nickase variant. In any aspect of the disclosure, or embodiments thereof, the napDNAbp contains a Cas9, Cas12a/Cpf1, Cas12b/C2c1, Cas12c/C2c3, Cas12d/CasY, Cas12e/CasX, Cas12g, Cas12h, Cas12i, or Cas12j/CasΦ polypeptide or a portion thereof. In any aspect of the disclosure, or embodiments thereof, where the napDNAbp contains a Cas9 polynucleotide or a portion thereof. In any aspect of the disclosure, or embodiments thereof, the napDNAbp contains a dead Cas9 (dCas9) or a Cas9 nickase (nCas9). In any aspect of the disclosure, or embodiments thereof, the napDNAbp is a modified Staphylococcus aureus Cas9 (SaCas9), Streptococcus thermophilus 1 Cas9 (St1Cas9), Streptococcus pyogenes Cas9 (SpCas9), or variants thereof. In any aspect of the disclosure, or embodiments thereof, the napDNAbp contains a variant of SpCas9 having an altered protospacer-adjacent motif (PAM) specificity. In any aspect of the disclosure, or embodiments thereof, the altered PAM has specificity for the nucleic acid sequence 5′-NGC-3′. In any aspect of the disclosure, or embodiments thereof, the napDNAbp recognizes an NRCH PAM sequence, where R is A or G, and His A, C, or T. In any aspect of the disclosure, or embodiments thereof, the napDNAbp recognizes the PAM nucleotide sequence CACC. In any aspect of the disclosure, or embodiments thereof, the napDNAbp contains a nucleotide sequence with at least 85% sequence identity to the following amino acid sequence: DKKYSIGLAIGTNSVGWAVITDEYKVPSKKFKVLGNTDRHSIKKNLIGALLFDSGETAEATRLK RTARRRYTRRKNRICYLQEIFSNEMAKVDDSFFHRLEESFLVEEDKKHERHPIFGNIVDEVAYH EKYPTIYHLRKKLVDSTDKADLRLIYLALAHMIKFRGHFLIEGDLNPDNSDVDKLFIQLVQTYN QLFEENPINASGVDAKAILSARLSKSRRLENLIAOLPGEKKNGLFGNLIALSLGLTPNFKSNFD LAEDAKLQLSKDTYDDDLDNLLAQIGDQYADLFLAAKNLSDAILLSDILRVNTEITKAPLSASM VKRYDEHHQDLTLLKALVRQQLPEKYKEIFFDQSKNGYAGYIDGGASQEEFYKFIKPILEKMDG TEELLVKLNREDLLRKORTFDNGIIPHQIHLGELHAILRRQGDFYPFLKDNREKIEKILTFRIP YYVGPLARGNSRFAWMTRKSEETITPWNFEEVVDKGASAQSFIERMTNFDKNLPNEKVLPKHSL LYEYFTVYNELTKVKYVTEGMRKPAFLSGEQKKAIVDLLFKTNRKVTVKOLKEDYFKKIECFDS VEISGVEDRFNASLGTYHDLLKIIKDKDFLDNEENEDILEDIVLTLTLFEDREMIEERLKTYAH LFDDKVMKQLKRLRYTGWGRLSRKLINGIRDKQSGKTILDFLKSDGFANRNFMQLIHDDSLTFK EDIQKAQVSGQGDSLHEHIANLAGSPAIKKGILQTVKVVDELVKVMGGHKPENIVIEMARENOT TQKGQKNSRERMKRIEEGIKELGSQILKEHPVENTQLQNEKLYLYYLQNGRDMYVDQELDINRL SDYDVDHIVPQSFLKDDSIDNKVLTRSDKNRGKSDNVPSEEVVKKMKNYWRQLLNAKLITQRKF DNLTKAERGGLSELDKAGFIKRQLVETRQITKHVAQILDSRMNTKYDENDKLIREVKVITLKSK LVSDFRKDFQFYKVREINNYHHAHDAYLNAVVGTALIKKYPKLESEFVYGDYKVYDVRKMIAKS EQEIGKATAKYFFYSNIMNFFKTEITLANGEIRKRPLIETNGETGEIVWDKGRDFATVRKVLSM PQVNIVKKTEVQTGGFSKESILPKGNSDKLIARKKDWDPKKYGGFNSPTVAYSVLVVAKVEKGK SKKLKSVKELLGITIMERSSFEKNPIDFLEAKGYKEVKKDLIIKLPKYSLFELENGRKRMLASA GVLQKGNELALPSKYVNFLYLASHYEKLKGSPEDNEQKOLFVEQHKHYLDEIIEQISEFSKRVI LADANLDKVLSAYNKHRDKPIREQAENIIHLFTLTNLGAPAAFKYFDTTINRKQYNTTKEVLDA TLIRQSITGLYETRIDLSQLG (SEQ ID NO: 938), and recognizes a CACC PAM sequence.

In any aspect of the disclosure, or embodiments thereof, the method involves administering to the subject an antibody or antigen binding fragment thereof, where the antibody is selected from one or more of ABTx025, ABTx030, ABTx052, ABTx061, ABTx062, ABTx070, ABTx071, ABTx196, ABTx198, ABTx202, ABTx203, ABTx205, ABTx206, ABTx248, ABTx250, ABTx251, ABTx253, ABTx254, ABTx255, ABTx256, ABTx265, ABTx268, ABTx270, ABTx271, ABTx272, ABTx273, ABTx274, ABTx307, ABTx308, ABTx309, and ABTx313.

In any aspect of the disclosure, or embodiments thereof, the subject has a hemoglobinopathy. In embodiments, the hemoglobinopathy is selected from one or more of sickle cell anemia, thalassemia, Fanconi anemia, aplastic anemia, and Wiskott-Aldrich syndrome.

In any aspect of the disclosure, or embodiments thereof, the method further involves contacting the hematopoietic stem cell or progenitor thereof with a guide polynucleotide that targets a nucleic acid molecule encoding a beta globin (HBB) polypeptide, thereby introducing an amino acid alteration to an alanine at position 6 of the HBB polypeptide. In any aspect of the disclosure, or embodiments thereof, the method further involves contacting the hematopoietic stem cell or progenitor thereof with a guide polynucleotide that targets the base editor to effect a deamination of a nucleobase of a hemoglobin subunit gamma 1 and/or 2 (HBG1/2) promoter. In any aspect of the disclosure, or embodiments thereof, deamination of the nucleobase disrupts repressor binding to the hemoglobin subunit gamma 1 and/or 2 (HBG1/2) promoter. In any aspect of the disclosure, or embodiments thereof, deamination of the nucleobase effects an increase in gamma globin (HbF) expression.

In any aspect of the disclosure, or embodiments thereof, the guide polynucleotide contacting the CD117 polynucleotide contains a nucleotide sequence selected from one or more of: AUAAUAGCUGGCAUCACGGU (SEQ ID NO: 693; gRNA931; CC200); CCACUAGCUUUCCAAACGGU (SEQ ID NO: 694; gRNA889); GCUGAACUGAUAGUCAACGU (SEQ ID NO: 695; gRNA908); UUUGACAAAGCCCGGAUCAG (SEQ ID NO: 696; gRNA918); UGAAAGUGAGGCCAGGUACU (SEQ ID NO: 697; gRNA923); AAACAGUCAGGUGAGUGAAU (SEQ ID NO: 698; gRNA928); AACUACAGGAGAAAUAUAAU (SEQ ID NO: 699; gRNA929); and GAUUAAAAGGCACCGAAGGA (SEQ ID NO: 700; gRNA944).

In any aspect of the disclosure, or embodiments thereof, the guide polynucleotide targeting deamination of a nucleobase of a HBG1/2 promoter, or the guide polynucleotide targeting a nucleic acid molecule encoding a beta globin (HBB) polypeptide contains a nucleotide sequence selected from one or more of: ACUUCUCCACAGGAGUCAGG (SEQ ID NO: 902); GUGGGGAAGGGGCCCCCAAG (SEQ ID NO: 903); AUUGAGAUAGUGUGGGGAAG (SEQ ID NO: 904); CAUUGAGAUAGUGUGGGGAA (SEQ ID NO: 905); GCAUUGAGAUAGUGUGGGGA (SEQ ID NO: 906); GUGGGGAAGGGGCCCCCAAG (SEQ ID NO: 907); GCUAUUGGUCAAGGCAAGGC (SEQ ID NO: 908); CAAGGCUAUUGGUCAAGGCA (SEQ ID NO: 909); CUUGUCAAGGCUAUUGGUCA (SEQ ID NO: 910); CUUGACCAAUAGCCUUGACA (SEQ ID NO: 911); GUUUGCCUUGUCAAGGCUAU (SEQ ID NO: 912); UGGUCAAGUUUGCCUUGUCA (SEQ ID NO: 913); UGGGGAAGGGGCCCCCAAGA (SEQ ID NO: 914); GUGUGGGGAAGGGGCCCCCA (SEQ ID NO: 915); UCAGACAGAUAUUUGCAUUG (SEQ ID NO: 916); UUUCAGACAGAUAUUUGCAU (SEQ ID NO: 917); CUUGCCUUGACCAAUAGCCU (SEQ ID NO: 918); UAGCCUUGACAAGGCAAACU (SEQ ID NO: 919); CAAACUUGACCAAUAGUCUU (SEQ ID NO: 920); UGUGGGGAAGGGGCCCCCAA (SEQ ID NO: 921); GGGCCCCUUCCCCACACUAU (SEQ ID NO: 922); CAGACAGAUAUUUGCAUUGA (SEQ ID NO: 923); UUUCAGACAGAUAUUUGCAU (SEQ ID NO: 924); GCCUUGACAAGGCAAACUUG (SEQ ID NO: 925); UUGACAAGGCAAACUUGACC (SEQ ID NO: 926); UGACCAAUAGUCUUAGAGUA (SEQ ID NO: 927); and AGACAGAUAUUUGCAUUGAGAUA (SEQ ID NO: 928). In any aspect of the disclosure, or embodiments thereof, the guide polynucleotide contains a scaffold with the following nucleotide sequence:

(SEQ ID NO: 317)
GUUUUAGAGCUAGAAAUAGCAAGUUAAAAUAAGGCUAGUCCGUUAUCAA
CUUGAAAAAGUGGCACCGAGUCGGUGCUUUU.

In any aspect of the disclosure, or embodiments thereof, the hematopoietic stem cell or progenitor thereof is autologous to the subject. In any aspect of the disclosure, or embodiments thereof, the hematopoietic stem cell or progenitor thereof is allogeneic to the subject. In any aspect of the disclosure, or embodiments thereof, the subject is a mammal. In any aspect of the disclosure, or embodiments thereof, the mammal is a canine, feline, human, non-human primate, or rodent.

In any aspect of the disclosure, or embodiments thereof, the guide polynucleotide is a guide RNA. In any aspect of the disclosure, or embodiments thereof, at least one of the two or more guide polynucleotides contain a nucleotide sequence selected from one or more of: AUAAUAGCUGGCAUCACGGU (SEQ ID NO: 693; gRNA931; CC200); CCACUAGCUUUCCAAACGGU (SEQ ID NO: 694; gRNA889); GCUGAACUGAUAGUCAACGU (SEQ ID NO: 695; gRNA908); UUUGACAAAGCCCGGAUCAG (SEQ ID NO: 696; gRNA918); UGAAAGUGAGGCCAGGUACU (SEQ ID NO: 697; gRNA923); AAACAGUCAGGUGAGUGAAU (SEQ ID NO: 698; gRNA928); AACUACAGGAGAAAUAUAAU (SEQ ID NO: 699; gRNA929); and GAUUAAAAGGCACCGAAGGA (SEQ ID NO: 700; gRNA944). In any aspect of the disclosure, or embodiments thereof, at least one of the two or more guide polynucleotides contains a nucleotide sequence selected from one or more of: ACUUCUCCACAGGAGUCAGG (SEQ ID NO: 902); GUGGGGAAGGGGCCCCCAAG (SEQ ID NO: 903); AUUGAGAUAGUGUGGGGAAG (SEQ ID NO: 904); CAUUGAGAUAGUGUGGGGAA (SEQ ID NO: 905); GCAUUGAGAUAGUGUGGGGA (SEQ ID NO: 906); GUGGGGAAGGGGCCCCCAAG (SEQ ID NO: 907); GCUAUUGGUCAAGGCAAGGC (SEQ ID NO: 908); CAAGGCUAUUGGUCAAGGCA (SEQ ID NO: 909); CUUGUCAAGGCUAUUGGUCA (SEQ ID NO: 910); CUUGACCAAUAGCCUUGACA (SEQ ID NO: 911); GUUUGCCUUGUCAAGGCUAU (SEQ ID NO: 912); UGGUCAAGUUUGCCUUGUCA (SEQ ID NO: 913); UGGGGAAGGGGCCCCCAAGA (SEQ ID NO: 914); GUGUGGGGAAGGGGCCCCCA (SEQ ID NO: 915); UCAGACAGAUAUUUGCAUUG (SEQ ID NO: 916); UUUCAGACAGAUAUUUGCAU (SEQ ID NO: 917); CUUGCCUUGACCAAUAGCCU (SEQ ID NO: 918); UAGCCUUGACAAGGCAAACU (SEQ ID NO: 919); CAAACUUGACCAAUAGUCUU (SEQ ID NO: 920); UGUGGGGAAGGGGCCCCCAA (SEQ ID NO: 921); GGGCCCCUUCCCCACACUAU (SEQ ID NO: 922); CAGACAGAUAUUUGCAUUGA (SEQ ID NO: 923); UUUCAGACAGAUAUUUGCAU (SEQ ID NO: 924); GCCUUGACAAGGCAAACUUG (SEQ ID NO: 925); UUGACAAGGCAAACUUGACC (SEQ ID NO: 926); UGACCAAUAGUCUUAGAGUA (SEQ ID NO: 927); and AGACAGAUAUUUGCAUUGAGAUA (SEQ ID NO: 928). In any aspect of the disclosure, or embodiments thereof, the guide polynucleotide is selected from one or more of: AUAAUAGCUGGCAUCACGGU (SEQ ID NO: 693; gRNA931; CC200); CCACUAGCUUUCCAAACGGU (SEQ ID NO: 694; gRNA889); GCUGAACUGAUAGUCAACGU (SEQ ID NO: 695; gRNA908); UUUGACAAAGCCCGGAUCAG (SEQ ID NO: 696; gRNA918); UGAAAGUGAGGCCAGGUACU (SEQ ID NO: 697; gRNA923); AAACAGUCAGGUGAGUGAAU (SEQ ID NO: 698; gRNA928); AACUACAGGAGAAAUAUAAU (SEQ ID NO: 699; gRNA929); and GAUUAAAAGGCACCGAAGGA (SEQ ID NO: 700; gRNA944).

In any aspect of the disclosure, or embodiments thereof, the base editor system further contains a guide polynucleotide containing the following polynucleotide sequence:

(SEQ ID NO: 902)
ACUUCUCCACAGGAGUCAGG.

In any aspect of the disclosure, or embodiments thereof, the antibody contains a heavy chain variable domain (VH) sequence having at least 85% amino acid sequence identity to the amino acid sequence: EVQLVESGGGLVQPGRSLRLSCAASGFTFDDYAMHWVRQAPGKGLEWVSGISWNSGTIGYADSV KGRFTNSRDNAKNSLYLEMNSLRAEDTALYYCAKDSPPGYCSSASCYGAFDIWGQGTMVTVSS (SEQ ID NO: 1120; ABTx025 VH), and/or containing a light chain variable domain (VL) sequence having at least 85% amino acid sequence identity to the amino acid sequence:

(SEQ ID NO: 1121; ABTx025 VL)
DVVMTQSPGTLSLSPGERATLSCRASQSVSSSYLAWYQQKPGQAPRLLI
YGASSRATGIPDRFSGSGSGTDFTLTISRLEPEDFAVYYCQQYGTSLTF
GGGTKLEIKRTV.

In any aspect of the disclosure, or embodiments thereof, the antibody contains a heavy chain variable domain (VH) sequence having at least 85% amino acid sequence identity to the amino acid sequence: QVQLQESGGGLVKPGGSLRLSCAASGFTFSSYSMNWVRQAPGKGLEWVSSIGTISSYIYYADSV KGRFTISRDNAKNSLYLQMNSLRAEDTAVYYCARDYYGGLFDYWGQGTLVTVSS (SEQ ID NO: 1118; ABTx030 VH), and/or contains a light chain variable domain (VL) sequence having at least 85% amino acid sequence identity to the amino acid sequence:

(SEQ ID NO: 1119; ABTx030 VL)
EIVMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVDIKRTV.

In any aspect of the disclosure, or embodiments thereof, the antibody contains a heavy chain variable domain (VH) sequence having at least 85% amino acid sequence identity to the amino acid sequence: EVOLVESGGGLVQPGRSLRLSCAASGFTFDDYAMHWVRQAPGKGLEWVSGISWNSGSIGYADSV KGRFTISRDNAKNSLYLQMNSLRAEDTALYYCAKDTPLGYCSTTSCYGAFDIWGQGTMVTVSS (SEQ ID NO: 1126; ABTx061 VH), and/or contains a light chain variable domain (VL) sequence having at least 85% amino acid sequence identity to the amino acid sequence:

(SEQ ID NO: 1127; ABTx061 VL)
EIVLAQSPGTLSLSPGERATLSCRASQSVSSSYLAWYQQKPGQAPRLLI
YGASSRATGIPDRFSGSGSGTDFTLTISRLEPEDFAVYYCQQYGSSPLT
FGGGTKLEIKRTV.

In any aspect of the disclosure, or embodiments thereof, the antibody contains a heavy chain variable domain (VH) sequence having at least 85% amino acid sequence identity to the amino acid sequence: EVOLVESGGGLVQPGRSLRLSCSASGFTFDDYAMHWVRQAPGKGLEWVSGISWNSGTIGYADSV KGRFTISRDNAKNSLYLQMNSLRAEDTALYYCAKDSPPGYCSSASCYGAFDIWGQGTTVTVSS (SEQ ID NO: 1128; ABTx062 VH), and/or contains a light chain variable domain (VL) sequence having at least 85% amino acid sequence identity to the amino acid sequence:

(SEQ ID NO: 1129; ABTx062 VL)
DIQLTQSPSSLSASVGDRVTITCRASQSISSYLNWYQQKPGKAPKLLIY
AASSLQSGVPSRFSGSGSGTDFTLTINSLQPEDFATYYCQQSYSTPFTF
GPGTKVEIKRTV.

In any aspect of the disclosure, or embodiments thereof, the antibody contains a heavy chain variable domain (VH) sequence having at least 85% amino acid sequence identity to the amino acid sequence: EVOLVESGGGVVRPGGSLRLSCAASGFTFDDYGMSWVRQAPGKGLEWVSDINWNGGSTGYADSV KGRFTISRDNAKNSLYLQMNSLRAEDTALYYCARESWDGSGIYYMDVWGKGTTVTVSS (SEQ ID NO: 1130; ABTx070 VH), and/or contains a light chain variable domain (VL) sequence having at least 85% amino acid sequence identity to the amino acid sequence:

(SEQ ID NO: 1131; ABTx070 VL)
DIVMTQSPSFLSASVGDRVTITCRASQGISSYLNWYQQKPGKAPKLLIY
AASTLQNGVPSRFSGSGSGTDFTLTISSLQPEDFATYYCQQSYSTPITF
GQGTRLEIKRTV.

In any aspect of the disclosure, or embodiments thereof, the antibody contains a heavy chain variable domain (VH) sequence having at least 85% amino acid sequence identity to the amino acid sequence: EVOLVESGGGVVRPGGSLRLSCAASGFTFDDYGMSWVRQAPGKGLEWVSGINWNGGSTGYADSV KGRFTISRDNAKNSLHLQMNSLRAEDTALYYCARESWNYEGYYYMDVWGKGTTVTVSS (SEQ ID NO: 1132; ABTx071 VH), and/or contains a light chain variable domain (VL) sequence having at least 85% amino acid sequence identity to the amino acid sequence:

(SEQ ID NO: 1133; ABTx071 VL)
AIQMTQSPSFLSASVGDRVTITCRASQGISSYLAWYQQKPGKAPKLLIY
AASTLONGVPSRFSGSGSGTEFTLTISSLOPEDFATYYCQQLNSYPYTF
GQGTKLEIKRTV.

In any aspect of the disclosure, or embodiments thereof, the antibody contains a heavy chain variable domain (VH) sequence having at least 85% amino acid sequence identity to the amino acid sequence: QVQLVQSGAAVKKPGESLKISCKGSGYRFTSYWIGWVRQMPGKGLEWMGIIYPGDSDTRYSPSF QGQVTISAGKSISTAYLOWSSLKASDTAMYYCARHGRGYDGYDGAFDIWGQGTMVTVSS (SEQ ID NO: 1122; ABTx313 VH), and/or contains a light chain variable domain (VL) sequence having at least 85% amino acid sequence identity to the amino acid sequence:

(SEQ ID NO: 1123; ABTx313 VL)
AIQLTQSPSSLSASVGDRVTITCRASQGISSALAWYQQKPGKAPKLLIY
DASSLESGVPSRFSGSGSGTDFTLTISSLQPEDFATYYCQQFSSYPLTF
GGGTKVEIKRTV.

In any aspect of the disclosure, or embodiments thereof, the antibody contains a heavy chain variable domain (VH) sequence having at least 85% amino acid sequence identity to the amino acid sequence: QVQLVQSGAAVKKPGESLKISCKGSGYRFTSYWIGWVRQMPGKGLEWMGIIYPGDSDTRYSPSF QGQVTISAGKSISTAYLQWSSLKASDTAMYYCARHGRGYDGYDGAFDIWGQGTMVTVSS (SEQ ID NO: 1122; ABTx307 VH), and/or containing a light chain variable domain (VL) sequence having at least 85% amino acid sequence identity to the amino acid sequence:

(SEQ ID NO: 960; ABTx307 VL)
AIQLTQSPSSLSASVGDRVTITCRASQGISSALAWYQQKPGKAPKLLIY
DASSLESGVPSRFSGSGSGTDFTLTISSLQPEDFATYYCQQFNSYPLTF
GGGTKVEIKRTV.

In any aspect of the disclosure, or embodiments thereof, the antibody contains a heavy chain variable domain (VH) sequence having at least 85% amino acid sequence identity to the amino acid sequence: QVQLVQSGAAVKKPGESLKISCKGSGYRFTSYWIGWVRQMPGKGLEWMGIIYPGDSDTKYSPSF QGQVTISAGKSISTAYLQWSSLKASDTAMYYCARHGRGYDAYDGAFDIWGQGTMVTVSS (ID NO: 1124; ABTx308 VH), and/or contains a light chain variable domain (VL) sequence having at least 85% amino acid sequence identity to the amino acid sequence:

(SEQ ID NO: 1123; ABTx308 VL)
AIQLTQSPSSLSASVGDRVTITCRASQGISSALAWYQQKPGKAPKLLIY
DASSLESGVPSRFSGSGSGTDFTLTISSLQPEDFATYYCQQFSSYPLTF
GGGTKVEIKRTV.

In any aspect of the disclosure, or embodiments thereof, the antibody contains a heavy chain variable domain (VH) sequence having at least 85% amino acid sequence identity to the amino acid sequence: QVQLVQSGAAVKKPGESLKISCKGSGYRFTSYWIGWVRQMPGKGLEWMGIIYPGDSDTRYSPSF QGQVTISAGKSISTAYLOWSSLKASDTAMYYCARHGRGYDAYDGAFDIWGQGTMVTVSS (SEQ ID NO: 1124; ABTx309 VH), and/or contains a light chain variable domain (VL) sequence having at least 85% amino acid sequence identity to the amino acid sequence:

(SEQ ID NO: 1125; ABTx309 VL)
AIQLTQSPSSLSASVGDRVTITCRASQGISSALAWYQQKPGKAPKLLIY
DASSLESGVPSRFSGSGSGTDFTLTISSLQPEDFATYYCQQTNSYPLTF
GGGTKVEIKRTV.

In any aspect of the disclosure, or embodiments thereof, the antibody contains a heavy chain variable domain (VH) sequence having at least 85% amino acid sequence identity to the amino acid sequence: EVOLVESGGGLVQPGRSLRLSCAASGFTFDDYAMHWVRQAPGKGLEWVSGISWNSGTIGYADSV KGRFTNSRDNAKNSLYLEMNSLRAEDTALYYCAKDSPPGYCSSASCYGAFDIWGQGTMVTVSS (SEQ ID NO: 1120; ABTx196 VH), and/or contains a light chain variable domain (VL) sequence having at least 85% amino acid sequence identity to the amino acid sequence:

(SEQ ID NO: 1068; ABTx196 VL)
DVVMTQSPGTLSLSPGERATLSCRASQSVSSSYLAWYQQKPGQAPRLLI
YGASSRATGIPDRFSGSGSGTDFTLTISRLEPEDFAVYYCQQSETCLTF
GGGTKLEIK.

In any aspect of the disclosure, or embodiments thereof, the antibody contains a heavy chain variable domain (VH) sequence having at least 85% amino acid sequence identity to the amino acid sequence: EVOLVESGGGLVQPGRSLRLSCAASGFTFDDYAMHWVRQAPGKGLEWVSGISWNSGTIGYADSV KGRFTSSRDNAKNSLYLEMNSLRAEDTALYYCAKDSPPGYCSSASCYGAFDIWGQGTMVTVSS (SEQ ID NO: 1085; ABTx202 VH), and/or contains a light chain variable domain (VL) sequence having at least 85% amino acid sequence identity to the amino acid sequence:

(SEQ ID NO: 1086; ABTx202 VL)
EIVMTQSPGTLSLSPGERATLSCRASQSVSSSYLAWYQQKPGQAPRLLI
YGSSTRATGIPARFSGSGSGTEFALTISSLSEDFAVYYCQQYNFWPYT
FGQGTKVEIK.

In any aspect of the disclosure, or embodiments thereof, the antibody contains a heavy chain variable domain (VH) sequence having at least 85% amino acid sequence identity to the amino acid sequence: EVOLVESGGGLVQPGRSLRLSCAASGFTFDDYAMHWVRQAPGKGLEWVSGISWNSGTIGYADSV KGRFTNSRDNAKNSLYLEMNSLRAEDTALYYCAKDSPPGYCASASCYGAFDIWGQGTMVTVSS (SEQ ID NO: 1089; ABTx198 VH), and/or contains a light chain variable domain (VL) sequence having at least 85% amino acid sequence identity to the amino acid sequence:

(SEQ ID NO: 1103; ABTx198 VL)
DVVMTQSPGTLSLSPGERATLSCRASQSVSSSYLAWYQQKPGQAPRLLI
YGASSRATGIPDRFSGSGSGTDFTLTISRLEPEDFAVYYCQQDSLGLTF
GGGTKLEIK .

In any aspect of the disclosure, or embodiments thereof, the antibody contains a heavy chain variable domain (VH) sequence having at least 85% amino acid sequence identity to the amino acid sequence: EVOLVESGGGLVQPGRSLRLSCAASGFTFDDYAMHWVRQAPGKGLEWVSGISWNSGTIGYADSV KGRFTNSRDNAKNSLYLEMNSLRAEDTALYYCAKDWPSGFCSSAYCYGAFDIWGQGTMVTVSS (SEQ ID NO: 1092; ABTx203, VH), and/or contains a light chain variable domain (VL) sequence having at least 85% amino acid sequence identity to the amino acid sequence:

(SEQ ID NO: 1093; ABTx203, VL)
DIVMTQSPSFLSASVGDRVTITCRASQGISSYLAWYQQKPGKAPKLLIY
AASTLQNGVPSRFSGSGSGTDFTLTITSLQPEDFATYYCQQSYSTPYTF
GQGTKLEIK.

In any aspect of the disclosure, or embodiments thereof, the antibody contains a heavy chain variable domain (VH) sequence having at least 85% amino acid sequence identity to the amino acid sequence: EVOLVESGGGLVQPGRSLRLSCAASGFTFDDYAMHWVRQAPGKGLEWVSGISWNSGTIGYADSV KGRFTNSRDNAKNSLYLEMNSLRAEDTALYYCAKDSPPGYCSSASCYGAFDIWGQGTMVTVSS (SEQ ID NO: 1120; ABTx205 VH), and/or contains a light chain variable domain (VL) sequence having at least 85% amino acid sequence identity to the amino acid sequence:

(SEQ ID NO: 1093; ABTx205 VL)
DIVMTQSPSFLSASVGDRVTITCRASQGISSYLAWYQQKPGKAPKLLIY
AASTLQNGVPSRFSGSGSGTDFTLTITSLQPEDFATYYCQQSYSTPYTF
GQGTKLEIK

In any aspect of the disclosure, or embodiments thereof, the antibody contains a heavy chain variable domain (VH) sequence having at least 85% amino acid sequence identity to the amino acid sequence: EVQLVESGGGLVQPGRSLRLSCAASGFTFDDYAMHWVRQAPGKGLEWVSGISWNSGTIGYADSV KGRFTNSRDNAKNSLYLEMNSLRAEDTALYYCAKDSPPGYCSSASCYGAFDIRGQGTMVTVSS (SEQ ID NO: 1101; ABTx206 VH), and/or contains a light chain variable domain (VL) sequence having at least 85% amino acid sequence identity to the amino acid sequence:

(SEQ ID NO: 1093)
DIVMTQSPSFLSASVGDRVTITCRASQGISSYLAWYQQKPGKAPKLLIY
AASTLQNGVPSRFSGSGSGTDFTLTITSLQPEDFATYYCQQSYSTPYTF
GQGTKLEIK.

In any aspect of the disclosure, or embodiments thereof, the subject has a percent chimerism for the edited cells or cells derived or descended from the edited cells of at least about 10%, 20%, 30%, 40%, 50%, 60%, 70%, 80%, or 90% at 1 wk, 2 wks, 3 wks, 4 wks, 5 wks, 6 wks, 7 wks, 8 wks, 9 wks, 10 wks, 11 wks, and/or 12 wks following administration of the antibody or antigen binding fragment thereof. In embodiments, the percent chimerism is measured for bulk bone marrow, CD34+ cells, CD235a+ cells, CD19+ cells, and/or CD45+ cells.

In any aspect of the disclosure, or embodiments thereof, the method involves contacting a hematopoietic stem cell or progenitor thereof with a guide polynucleotide containing a spacer sequence corresponding to gRNA931 (CC200) and another guide polynucleotide containing a spacer sequence corresponding to sgRNA_027. In any aspect of the disclosure, or embodiments thereof, the base editor contains a TadA*8.20 adenosine deaminase domain. In any aspect of the disclosure, or embodiments thereof, the base editor contains a Cas9-NRCH napDNAbp domain. In any aspect of the disclosure, or embodiments thereof, the base editor is ABE8.20-NRCH. In any aspect of the disclosure, or embodiments thereof, the antibody is ABTx052, contains VH CDR1, VH CDR2, VH CDR3, VL CDR1, VL CDR2, and VL CDR3 of ABTx052, and/or contains a VH domain with at least 95% sequence identity to the VH domain of ABTx052 and a VL domain with at least 95% sequence identity to the VL domain of ABTx052. In any aspect of the disclosure, or embodiments thereof, the composition contains with a guide polynucleotide containing a spacer sequence corresponding to gRNA931 (CC200), a guide polynucleotide containing a spacer sequence corresponding to sgRNA_027, and/or an mRNA encoding the base editor ABE8.20-NRCH.

In any aspect of the disclosure, or embodiments thereof, the method involves (A) base editing hematopoietic stem cells or progenitor thereof by contacting them with (i) a guide polynucleotide containing a spacer sequence corresponding to gRNA931 (CC200) and another guide polynucleotide containing a spacer sequence corresponding to sgRNA_027, or polynucleotides encoding the same, and (ii) an ABE-NRCH base editor, or a polynucleotide encoding the same; (B) administering the base-edited hematopoietic stem cell or progenitor thereof to a subject; and (C) administering to the subject an ABTx052 antibody before, after, or concurrently with administration of the base-edited hematopoietic stem cells.

In any aspect of the disclosure, or embodiments thereof, the antibody, antibody drug conjugate, or chimeric antigen receptor contains complementarity determining regions (CDRs) that contain the following heavy chain variable region (VH) CDR and light chain variable region (VL) CDR amino acid sequences: VH CDR1: GYRFTSYW (SEQ ID NO: 421); VH CDR2 is IYPGDSDTR (SEQ ID NO: 958) or TYPGDSDTK (SEQ ID NO: 972); VH CDR3: ARHGRGYXYXGAFDI (SEQ ID NO: 944); VL CDR1: QGISSA (SEQ ID NO: 424); VL CDR2: DAS; and VL CDR3 is selected from one or more of QQXSYPLT (SEQ ID NO: 945), QQXSHPLT (SEQ ID NO: 946), and QQXSSPLT (SEQ ID NO: 947). X indicates any amino acid. The anti-CD117 antibody contains at least one amino acid alteration relative to the amino acid sequence of ABTx052.

In any aspect of the disclosure, or embodiments thereof, VH CDR3 is selected from one or more of ARHGRGYDGYEGAFDI (SEQ ID NO: 948), ARHGRGYNAYEGAFDI (SEQ ID NO: 949), ARHGRGYNGYDGAFDI (SEQ ID NO: 950), ARHGRGYDGYDGAFDI (SEQ ID NO: 1012), ARHGRGYDAYEGAFDI (SEQ ID NO: 1027), and ARHGRGYDAYDGAFDI (SEQ ID NO: 1032), and/or VL CDR3 is selected from one or more of QQTNSYPLT (SEQ ID NO: 951), QQVNSYPLT (SEQ ID NO: 952), QQFRSYPLT (SEQ ID NO: 953), QQFYSYPLT (SEQ ID NO: 954), QQFNSHPLT (SEQ ID NO: 955), QQFNSSPLT (SEQ ID NO: 956), QQFSSYPLT (SEQ ID NO: 1020), QQTNSHPLT (SEQ ID NO: 1023), QQVRSYPLT (SEQ ID NO: 1025), QQTYSYPLT (SEQ ID NO: 1029), QQFNSTPLT (SEQ ID NO: 1033), and QQTRSYPLT (SEQ ID NO: 1036).

In any aspect of the disclosure, or embodiments thereof, the antibody selectively binds wild type CD117. In any aspect of the disclosure, or embodiments thereof, the antibody has a rate of dissociation constant for binding to wild type CD117 that is less than about 4.0E-04 or 4.5.0E-03.

In any aspect of the disclosure, or embodiments thereof, the antibody has reduced binding to a CD117 variant containing a Y259C, N260D, and/or S261G amino acid alteration, where the reduced binding is relative to anti-CD117 antibody ABTx052 or ABTx135. In any aspect of the disclosure, or embodiments thereof, the antibody has reduced binding to a CD117 variant containing the amino acid alterations Y259C and N260D and to a CD117 variant containing the amino acid alteration S261G, where the reduced binding is relative to anti-CD117 antibody ABTx052 or ABTx135.

In any aspect of the disclosure, or embodiments thereof, the antibody contains variable heavy chain (VH) and variable light chain (VL) framework regions (FR) containing the following amino acid sequences: VH FR1: QVQLVQSGAAVKKPGESLKISCKGS (SEQ ID NO: 426); VH FR2: IGWVRQMPGKGLEWMGI (SEQ ID NO: 427); VH FR3: YSPSFQGQVTISAGKSISTAYLOWSSLKASDTAMYYC (SEQ ID NO: 973); VH FR4: WGQGTMVTVSS (SEQ ID NO: 429); VL FR1: AIQLTQSPSSLSASVGDRVTITCRAS (SEQ ID NO: 430); VL FR2: LAWYQQKPGKAPKLLIY (SEQ ID NO: 431); VL FR3: SLESGVPSRFSGSGSGTDFTLTISSLQPEDFATYYC (SEQ ID NO: 432); and VL FR4: FGGGTKVEIKRTV (SEQ ID NO: 433).

In any aspect of the disclosure, or embodiments thereof, the antibody is a human IgG1 antibody.

In any aspect of the disclosure, or embodiments thereof, the antibody or antigen binding fragment thereof, antibody drug conjugate, or a chimeric antigen receptor T (CAR-T) cell is administered before, after, or concurrently with the edited cell.

In any aspect provided herein, or embodiments thereof, the method is not a process for modifying the germline genetic identity of human beings.

In any aspect provided herein, or embodiments thereof, the antibody has an EC50 on a target cell of less than about 0.1 nM. In any aspect provided herein, or embodiments thereof, the antibody has polyspecificity that is similar to or lower than the polyspecificity of ABTx052. In any aspect provided herein, or embodiments thereof, the antibody is effective in reducing the viability of a hematopoietic stem cell (HSC) expressing a wild type CD117 polypeptide. In any aspect provided herein, or embodiments thereof, the antibody has an IC50 value for reducing viability of the HSC that is less than 5E-006 molar.

In any aspect of the disclosure, or embodiments thereof: A) VH CDR1 is selected from one or more of: GYRFTSYW (SEQ ID NO: 421), GFTFSSYS (SEQ ID NO: 406), GFTEDDYG (SEQ ID NO: 466), and GFTFDDYA (SEQ ID NO: 435); B) VH CDR2 is selected from one or more of: IYPGDSDTK (SEQ ID NO: 972), TYPGDSDTR (SEQ ID NO: 958), IGTISSYIY (SEQ ID NO: 1113), INWNGGSTG (SEQ ID NO: 1114), ISWNSGSIG (SEQ ID NO: 1115), and ISWNSGTIG (SEQ ID NO: 1070); C) VH CDR3 is selected from one or more of: ARHGRGYDAYDGAFDI (SEQ ID NO: 1032), ARHGRGYDGYDGAFDI (SEQ ID NO: 1012), ARDYYGGLFDY (SEQ ID NO: 408), ARESWDGSGIYYMDV (SEQ ID NO: 468), ARESWNYEGYYYMDV (SEQ ID NO: 483), AKDTPLGYCSTTSCYGAFDI (SEQ ID NO: 438), AKDWPSGFCSSAYCYGAFDI (SEQ ID NO: 1094), AKDSPPGYCSSASCYGAFDI (SEQ ID NO: 453), and AKDSPPGYCASASCYGAFDI (SEQ ID NO: 1090); D) VL CDR1 is selected from one or more of: QGISSA (SEQ ID NO: 424), QSISSY (SEQ ID NO: 409), QGISSY (SEQ ID NO: 469), and QSVSSSY (SEQ ID NO: 439); E) VL CDR2 is selected from one or more of: DAS, AAS, GSS, and GAS; and/or F) VL CDR3 is selected from one or more of: QQTNSYPLT (SEQ ID NO: 1044), QQFSSYPLT (SEQ ID NO: 1022), QQFNSYPLT (SEQ ID NO: 425), QQSYSTPLT (SEQ ID NO: 410), QQSYSTPFT (SEQ ID NO: 455), QQLNSYPYT (SEQ ID NO: 485), QQSYSTPIT (SEQ ID NO: 470), QQSYSTPYT (SEQ ID NO: 1097), QQYNFWPYT (SEQ ID NO: 1084), QQYGSSPLT (SEQ ID NO: 440), QQYGTSLT (SEQ ID NO: 395), QQSETCLT (SEQ ID NO: 1074), and QQDSLGLT (SEQ ID NO: 1091).

In any aspect of the disclosure, or embodiments thereof, the antibody contains variable heavy chain (VH) and variable light chain (VL) framework regions (FR), where A) VH FR1 is selected from one or more of: QVQLVQSGAAVKKPGESLKISCKGS (SEQ ID NO: 426), QVQLQESGGGLVKPGGSLRLSCAAS (SEQ ID NO: 411), EVOLVESGGGVVRPGGSLRLSCAAS (SEQ ID NO: 471), EVOLVESGGGLVQPGRSLRLSCAAS (SEQ ID NO: 396), and EVOLVESGGGLVQPGRSLRLSCSAS (SEQ ID NO: 456); B) VH FR2 is selected from one or more of: IGWVRQMPGKGLEWMGI (SEQ ID NO: 427), MNWVRQAPGKGLEWVSS (SEQ ID NO: 412), MSWVRQAPGKGLEWVSD (SEQ ID NO: 472), MSWVRQAPGKGLEWVSG (SEQ ID NO: 487), and MHWVRQAPGKGLEWVSG (SEQ ID NO: 397); C) VH FR3 is selected from one or more of: YSPSFQGQVTISAGKSISTAYLQWSSLKASDTAMYYC (SEQ ID NO: 973), YADSVKGRFTISRDNAKNSLYLOMNSLRAEDTAVYYC (SEQ ID NO: 413), YADSVKGRFTISRDNAKNSLYLQMNSLRAEDTALYYC (SEQ ID NO: 1116), YADSVKGRFTISRDNAKNSLHLQMNSLRAEDTALYYC (SEQ ID NO: 1117), YADSVKGRFTNSRDNAKNSLYLEMNSLRAEDTALYYC (SEQ ID NO: 1077), and YADSVKGRFTSSRDNAKNSLYLEMNSLRAEDTALYYC (SEQ ID NO: 1065); D) VH FR4 is selected from one or more of: WGQGTMVTVSS (SEQ ID NO: 389), WGQGTLVTVSS (SEQ ID NO: 414), WGKGTTVTVSS (SEQ ID NO: 474), WGQGTTVTVSS (SEQ ID NO: 459), and RGQGTMVTVSS (SEQ ID NO: 1102); E) VL FR1 is selected from one or more of: AIQLTQSPSSLSASVGDRVTITCRAS (SEQ ID NO: 430), EIVMTQSPSSLSASVGDRVTITCRAS (SEQ ID NO: 415), DIQLTQSPSSLSASVGDRVTITCRAS (SEQ ID NO: 460), AIQMTQSPSFLSASVGDRVTITCRAS (SEQ ID NO: 490), DIVMTQSPSFLSASVGDRVTITCRAS (SEQ ID NO: 475), EIVMTQSPGTLSLSPGERATLSCRAS (SEQ ID NO: 1087), EIVLAQSPGTLSLSPGERATLSCRAS (SEQ ID NO: 445), and DVVMTQSPGTLSLSPGERATLSCRAS (SEQ ID NO: 400); F) VL FR2 is selected from one or more of: LAWYQQKPGKAPKLLIY (SEQ ID NO: 431), LNWYQQKPGKAPKLLIY (SEQ ID NO: 416), and LAWYQQKPGQAPRLLIY (SEQ ID NO: 446); G) VL FR3 is selected from one or more of: SLESGVPSRFSGSGSGTDFTLTISSLOPEDFATYYC (SEQ ID NO: 432), SLQSGVPSRFSGSGSGTDFTLTISSLOPEDFATYYC (SEQ ID NO: 417), SLQSGVPSRFSGSGSGTDFTLTINSLOPEDFATYYC (SEQ ID NO: 462), TLQNGVPSRFSGSGSGTEFTLTISSLOPEDFATYYC (SEQ ID NO: 492), TLQNGVPSRFSGSGSGTDFTLTISSLQPEDFATYYC (SEQ ID NO: 477), TLQNGVPSRFSGSGSGTDFTLTITSLQPEDFATYYC (SEQ ID NO: 1099), TRATGIPARFSGSGSGTEFALTISSLQSEDFAVYYC (SEQ ID NO: 1080), and SRATGIPDRFSGSGSGTDFTLTISRLEPEDFAVYYC (SEQ ID NO: 402); and/or H) VL FR4 is selected from one or more of: FGGGTKVEIKRTV (SEQ ID NO: 433), FGGGTKVDIKRTV (SEQ ID NO: 418), FGPGTKVEIKRTV (SEQ ID NO: 463), FGQGTKLEIKRTV (SEQ ID NO: 493), FGQGTRLEIKRTV (SEQ ID NO: 478), FGQGTKLEIK (SEQ ID NO: 1100), FGQGTKVEIK (SEQ ID NO: 1088), FGGGTKLEIKRTV (SEQ ID NO: 403), and FGGGTKLEIK (SEQ ID NO: 1082).

In any aspect of the disclosure, or embodiments thereof, the hemoglobinopathy is selected from one or more of sickle cell anemia, thalassemia, Fanconi anemia, aplastic anemia, and Wiskott-Aldrich syndrome.

In any aspect of the disclosure, or embodiments thereof, step (b) takes place before, after, or concurrently with step (a).

In any aspect of the disclosure, or embodiments thereof, the CD117 polypeptide contains an S261G alteration.

In any aspect of the disclosure, or embodiments thereof, the sequence of consecutive amino acids has at least 90%, 95%, 99%, 99.5%, or 99.9% sequence identity to the fragment. In any aspect of the disclosure, or embodiments thereof, the CD117 polypeptide has at least 90%, 95%, 99%, 99.5%, or 99.9% sequence identity to the amino acid sequence.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “ABTx025” is meant an antibody having at least about 85% amino acid sequence identity to an antibody sequence of antibody ABTx025 or comprising VH and/or VL CDRs 1-3 of ABTx025 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx025. Exemplary heavy chain and light chain sequences for antibody ABTx025 are provided below, where the variable regions are in plain text, the constant domains are in bold, and complementarity determining regions (CDRs), i.e., CDR1, CDR2, and CDR2, are underlined:

ABTx025 heavy chain (HC):
(SEQ ID NO: 389)
EVQLVESGGGLVQPGRSLRLSCAASGFTFDDYAMHWVRQAPGKGLEWVS
GISWNSGTIGYADSVKGRFTNSRDNAKNSLYLEMNSLRAEDTALYYCAK
DSPPGYCSSASCYGAFDIWGQGTMVTVSSASTKGPSVFPLAPSSKSTSG
GTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEA
LHNHYTQKSLSLSPGK
ABTx025 light chain (LC):
(SEQ ID NO: 390)
DVVMTQSPGTLSLSPGERATLSCRASQSVSSSYLAWYQQKPGQAPRLLI
YGASSRATGIPDRFSGSGSGTDFTLTISRLEPEDFAVYYCQQYGTSLTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx025 antibody VH region are as follows:

VH CDR1:
(SEQ ID NO: 391)
GFTFDDYA;
VH CDR2:
(SEQ ID NO: 392)
ISWNSGTI;
and
VH CDR3:
(SEQ ID NO: 393)
AKDSPPGYCSSASCYGAFDI.

The three CDRs of the ABTx025 antibody VL region are as follows:

VL CDR1:
(SEQ ID NO: 394)
QSVSSSY;
VL CDR2:
GAS;
and
VL CDR3:
(SEQ ID NO: 395)
QQYGTSLT.

The four framework (FR) regions, i.e., FR1, FR2, FR3, and FR4, of the ABTx025 antibody are located on either side of each of the CDRs in VH and VL region sequences shown supra. In particular, the four FRs of the ABTx025 antibody VH region are as follows:

VH FR1:
(SEQ ID NO: 396)
EVQLVESGGGLVQPGRSLRLSCAAS;
VH FR2:
(SEQ ID NO: 397)
MHWVRQAPGKGLEWVSG;
VH FR3:
(SEQ ID NO: 398)
IGYADSVKGRFTNSRDNAKNSLYLEMNSLRAEDTALYYC;
and
VH FR4:
(SEQ ID NO: 399)
WGQGTMVTVSS.

The four FRs of the ABTx025 antibody VL region are as follows:

VL FR1:
(SEQ ID NO: 400)
DVVMTQSPGTLSLSPGERATLSCRAS;
VL FR2:
(SEQ ID NO: 401)
LAWYQQKPGQAPRLLIY;
VL FR3:
(SEQ ID NO: 402)
SRATGIPDRFSGSGSGTDFTLTISRLEPEDFAVYYC;
and
VL FR4:
(SEQ ID NO: 403)
FGGGTKLEIKRTV.

By “ABTx025 polynucleotide” is meant a nucleic acid molecule (e.g., DNA) encoding at least a fragment of an ABTx025 antibody. In an embodiment, the encoded fragment has antigen binding activity.

By “ABTx030” is meant an antibody having at least about 85% amino acid sequence identity to an antibody sequence of antibody ABTx030 or comprising VH and/or VL CDRs 1-3 of ABTx030 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx030. Exemplary heavy chain and light chain sequences for antibody ABTx030 are provided below, where the variable regions are in plain text, the constant domains are in bold, and complementarity determining regions (CDRs), i.e., CDR1, CDR2, and CDR2, are underlined:

ABTx030 heavy chain (HC):
(SEQ ID NO: 404)
QVQLQESGGGLVKPGGSLRLSCAASGFTFSSYSMNWVRQAPGKGLEWVS
SIGTISSYIYYADSVKGRFTISRDNAKNSLYLQMNSLRAEDTAVYYCAR
DYYGGLFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK
ABTx030 light chain (LC):
(SEQ ID NO: 405)
EIVMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVDIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx030 antibody VH region are as follows:

VH CDR1:
(SEQ ID NO: 406)
GFTFSSYS;
VH CDR2:
(SEQ ID NO: 407)
IGTISSYI
or
(SEQ ID NO: 1113)
IGTISSYIY;
and
VH CDR3:
(SEQ ID NO: 408)
ARDYYGGLFDY.

The three CDRs of the ABTx030 antibody VL region are as follows:

VL CDR1:
(SEQ ID NO: 409)
QSISSY;
VL CDR2:
AAS;
and
VL CDR3:
(SEQ ID NO: 410)
QQSYSTPLT.

The four framework (FR) regions, i.e., FR1, FR2, FR3, and FR4, of the ABTx030 antibody are located on either side of each of the CDRs in VH and VL region sequences shown supra. In particular, the four FRs of the ABTx030 antibody VH region are as follows:

VH FR1:
(SEQ ID NO: 411)
QVQLQESGGGLVKPGGSLRLSCAAS;
VH FR2:
(SEQ ID NO: 412)
MNWVRQAPGKGLEWVSS;
VH FR3:
(SEQ ID NO: 413)
YYADSVKGRFTISRDNAKNSLYLQMNSLRAEDTAVYYC;
and
VH FR4:
(SEQ ID NO: 414)
WGQGTLVTVSS.

The four FRs of the ABTx030 antibody VL region are as follows:

VL FR1:
(SEQ ID NO: 415)
EIVMTQSPSSLSASVGDRVTITCRAS;
VL FR2:
(SEQ ID NO: 416)
LNWYQQKPGKAPKLLIY;
VL FR3:
(SEQ ID NO: 417)
SLQSGVPSRFSGSGSGTDFTLTISSLQPEDFATYYC;
and
VL FR4:
(SEQ ID NO: 418)
FGGGTKVDIKRTV.

By “ABTx030 polynucleotide” is meant a nucleic acid molecule (e.g., DNA) encoding at least a fragment of an ABTx030 antibody. In an embodiment, the encoded fragment has antigen binding activity.

By “ABTx052” or “mAb-7” is meant an antibody having at least about 85% amino acid sequence identity to an antibody sequence of antibody ABTx052 or comprising VH and/or VL CDRs 1-3 of ABTx052 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx052.

Exemplary heavy chain and light chain sequences for antibody ABTx052 are provided below, where embodiments of the variable regions are in plain text, embodiments of the constant domains are in bold, and embodiments of complementarity determining regions (CDRs), i.e., CDR1, CDR2, and CDR2, are underlined:

ABTx052 heavy chain (HC):
(SEQ ID NO: 419)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYNGYEGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx052 light chain (LC):
(SEQ ID NO: 420)
AIQLTQSPSSLSASVGDRVTITCRASQGISSALAWYQQKPGKAPKLLIY
DASSLESGVPSRFSGSGSGTDFTLTISSLQPEDFATYYCQQFNSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx052 antibody VH region are as follows:

(SEQ ID NO: 421)
VH CDR1: GYRFTSYW;
(SEQ ID NO: 422)
VH CDR2: IYPGDSDT
or
(SEQ ID NO: 958)
IYPGDSDTR;
and
(SEQ ID NO: 423)
VH CDR3: ARHGRGYNGYEGAFDI.

The three CDRs of the ABTx052 antibody VL region are as follows:

(SEQ ID NO: 424)
VL CDR1: QGISSA;
VL CDR2: DAS;
and
(SEQ ID NO: 425)
VL CDR3: QQFNSYPLT.

The four framework (FR) regions, i.e., FR1, FR2, FR3, and FR4, of the ABTx052 antibody are located on either side of each of the CDRs in VH and VL region sequences shown supra. In particular, the four FRs of the ABTx052 antibody VH region are as follows:

(SEQ ID NO: 426)
VH FR1: QVQLVQSGAAVKKPGESLKISCKGS;
(SEQ ID NO: 427)
VH FR2: IGWVRQMPGKGLEWMGI;
and
(SEQ ID NO: 428)
VH FR3: RYSPSFQGQVTISAGKSISTAYLQWSSLKASDTAMYYC
or
(SEQ ID NO: 973)
YSPSFQGQVTISAGKSISTAYLQWSSLKASDTAMYYC;
(SEQ ID NO: 429)
VH FR4: WGQGTMVTVSS.

The four FRs of the ABTx052 antibody VL region are as follows:

(SEQ ID NO: 430)
VL FR1: AIQLTQSPSSLSASVGDRVTITCRAS;
(SEQ ID NO: 431)
VL FR2: LAWYQQKPGKAPKLLIY;
(SEQ ID NO: 432)
VL FR3: SLESGVPSRFSGSGSGTDFTLTISSLQPEDFATYYC;
and
(SEQ ID NO: 433)
VL FR4: FGGGTKVEIKRTV.

By “ABTx052 polynucleotide” is meant a nucleic acid molecule (e.g., DNA) encoding at least a fragment of an ABTx052 antibody. In an embodiment, the encoded fragment has antigen binding activity.

By “ABTx061” is meant an antibody having at least about 85% amino acid sequence identity to an antibody sequence of antibody ABTx061 or comprising VH and/or VL CDRs 1-3 of ABTx061 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx061. Exemplary heavy chain and light chain sequences for antibody ABTx061 are provided below, where the variable regions are in plain text, the constant domains are in bold, and complementarity determining regions (CDRs), i.e., CDR1, CDR2, and CDR2, are underlined:

ABTx061 heavy chain (HC):
(SEQ ID NO: 434)
EVQLVESGGGLVQPGRSLRLSCAASGFTFDDYAMHWVRQAPGKGLEWVSG
ISWNSGSIGYADSVKGRFTISRDNAKNSLYLQMNSLRAEDTALYYCAKDT
PLGYCSTTSCYGAFDIWGQGTMVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK
ABTx061 light chain (LC):
(SEQ ID NO: 435)
EIVLAQSPGTLSLSPGERATLSCRASQSVSSSYLAWYQQKPGQAPRLLIY
GASSRATGIPDRFSGSGSGTDFTLTISRLEPEDFAVYYCQQYGSSPLTFG
GGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

The three CDRs of the ABTx061 antibody VH region are as follows:

(SEQ ID NO: 436)
VH CDR1: GFTFDDYA;
(SEQ ID NO: 437)
VH CDR2: ISWNSGSI
or
ISWNSGSIG;
(SEQ ID NO: 1115)
and
(SEQ ID NO: 438)
VH CDR3: AKDTPLGYCSTTSCYGAFDI.

The three CDRs of the ABTx061 antibody VL region are as follows:

(SEQ ID NO: 439)
VL CDR1: QSVSSSY;
VL CDR2: GAS;
and
(SEQ ID NO: 440)
VL CDR3: QQYGSSPLT.

The four framework (FR) regions, i.e., FR1, FR2, FR3, and FR4, of the ABTx061 antibody are located on either side of each of the CDRs in VH and VL region sequences shown supra. In particular, the four FRs of the ABTx061 antibody VH region are as follows:

(SEQ ID NO: 441)
VH FR1: EVQLVESGGGLVQPGRSLRLSCAA;
(SEQ ID NO: 442)
VH FR2: MHWVRQAPGKGLEWVSG;
(SEQ ID NO: 443)
VH FR3: GYADSVKGRFTISRDNAKNSLYLQMNSLRAEDTALYYC
or
(SEQ ID NO: 1116)
YADSVKGRFTISRDNAKNSLYLQMNSLRAEDTALYYC;
and
(SEQ ID NO: 444)
VH FR4: WGQGTMVTVSS.

The four FRs of the ABTx061 antibody VL region are as follows:

(SEQ ID NO: 445)
VL FR1: EIVLAQSPGTLSLSPGERATLSCRAS;
(SEQ ID NO: 446)
VL FR2: LAWYQQKPGQAPRLLIY;
(SEQ ID NO: 447)
VL FR3: SRATGIPDRFSGSGSGTDFTLTISRLEPEDFAVYYC;
and
(SEQ ID NO: 448)
VL FR4: FGGGTKLEIKRTV.

By “ABTx061 polynucleotide” is meant a nucleic acid molecule (e.g., DNA) encoding at least a fragment of an ABTx061 antibody. In an embodiment, the encoded fragment has antigen binding activity.

By “ABTx062” is meant an antibody having at least about 85% amino acid sequence identity to an antibody sequence of antibody ABTx062 or comprising VH and/or VL CDRs 1-3 of ABTx062 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx062. Exemplary heavy chain and light chain sequences for antibody ABTx062 are provided below, where the variable regions are in plain text, the constant domains are in bold, and complementarity determining regions (CDRs), i.e., CDR1, CDR2, and CDR2, are underlined:

ABTx062 heavy chain (HC):
(SEQ ID NO: 449)
EVQLVESGGGLVQPGRSLRLSCSASGFTFDDYAMHWVRQAPGKGLEWVSG
ISWNSGTIGYADSVKGRFTISRDNAKNSLYLQMNSLRAEDTALYYCAKDS
PPGYCSSASCYGAFDIWGQGTTVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK
ABTx062 light chain (LC):
(SEQ ID NO: 450)
DIQLTQSPSSLSASVGDRVTITCRASQSISSYLNWYQQKPGKAPKLLIYA
ASSLQSGVPSRFSGSGSGTDFTLTINSLQPEDFATYYCQQSYSTPFTFGP
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The three CDRs of the ABTx062 antibody VH region are as follows:

(SEQ ID NO: 451)
VH CDR1: GFTFDDYA;
(SEQ ID NO: 452)
VH CDR2: ISWNSGTI;
and
(SEQ ID NO: 453)
VH CDR3: AKDSPPGYCSSASCYGAFDI.

The three CDRs of the ABTx062 antibody VL region are as follows:

(SEQ ID NO: 454)
VL CDR1: QSISSY;
VL CDR2: AAS;
and
(SEQ ID NO: 455)
VL CDR3: QQSYSTPFT.

The four framework (FR) regions, i.e., FR1, FR2, FR3, and FR4, of the ABTx062 antibody are located on either side of each of the CDRs in VH and VL region sequences shown supra. In particular, the four FRs of the ABTx062 antibody VH region are as follows:

(SEQ ID NO: 456)
VH FR1: EVQLVESGGGLVQPGRSLRLSCSAS;
(SEQ ID NO: 457)
VH FR2: MHWVRQAPGKGLEWVSG;
(SEQ ID NO: 458)
VH FR3: GYADSVKGRFTISRDNAKNSLYLQMNSLRAEDTALYYC
or
(SEQ ID NO: 1116)
YADSVKGRFTISRDNAKNSLYLQMNSLRAEDTALYYC;
and
(SEQ ID NO: 459)
VH FR4: WGQGTTVTVSS.

The four FRs of the ABTx062 antibody VL region are as follows:

(SEQ ID NO: 460)
VL FR1: DIQLTQSPSSLSASVGDRVTITCRAS;
(SEQ ID NO: 461)
VL FR2: LNWYQQKPGKAPKLLIY;
(SEQ ID NO: 462)
VL FR3: SLQSGVPSRFSGSGSGTDFTLTINSLQPEDFATYYC;
and
(SEQ ID NO: 463)
VL FR4: FGPGTKVEIKRTV.

By “ABTx062 polynucleotide” is meant a nucleic acid molecule (e.g., DNA) encoding at least a fragment of an ABTx062 antibody. In an embodiment, the encoded fragment has antigen binding activity.

By “ABTx070” is meant an antibody having at least about 85% amino acid sequence identity to an antibody sequence of antibody ABTx070 or comprising VH and/or VL CDRs 1-3 of ABTx070 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx070. Exemplary heavy chain and light chain sequences for antibody ABTx070 are provided below, where the variable regions are in plain text, the constant domains are in bold, and complementarity determining regions (CDRs), i.e., CDR1, CDR2, and CDR2, are underlined:

ABTx070 heavy chain (HC):
(SEQ ID NO: 464)
EVQLVESGGGVVRPGGSLRLSCAASGFTFDDYGMSWVRQAPGKGLEWVSD
INWNGGSTGYADSVKGRFTISRDNAKNSLYLQMNSLRAEDTALYYCARES
WDGSGIYYMDVWGKGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK
ABTx070 light chain (LC):
(SEQ ID NO: 465)
DIVMTQSPSFLSASVGDRVTITCRASQGISSYLNWYQQKPGKAPKLLIYA
ASTLQNGVPSRFSGSGSGTDFTLTISSLQPEDFATYYCQQSYSTPITFGQ
GTRLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The three CDRs of the ABTx070 antibody VH region are as follows:

(SEQ ID NO: 466)
VH CDR1: GFTFDDYG;
(SEQ ID NO: 467)
VH CDR2: INWNGGST
or
(SEQ ID NO: 1114)
INWNGGSTG;
and
(SEQ ID NO: 468)
VH CDR3: ARESWDGSGIYYMDV.

The three CDRs of the ABTx070 antibody VL region are as follows:

(SEQ ID NO: 469)
VL CDR1: QGISSY;
VL CDR2: AAS;
and
(SEQ ID NO: 470)
VL CDR3: QQSYSTPIT.

The four framework (FR) regions, i.e., FR1, FR2, FR3, and FR4, of the ABTx070 antibody are located on either side of each of the CDRs in VH and VL region sequences shown supra. In particular, the four FRs of the ABTx070 antibody VH region are as follows:

(SEQ ID NO: 471)
VH FR1: EVQLVESGGGVVRPGGSLRLSCAAS;
(SEQ ID NO: 472)
VH FR2: MSWVRQAPGKGLEWVSD;
(SEQ ID NO: 473)
VH FR3: GYADSVKGRFTISRDNAKNSLYLQMNSLRAEDTALYYC
or
(SEQ ID NO: 1116)
YADSVKGRFTISRDNAKNSLYLQMNSLRAEDTALYYC;
and
(SEQ ID NO: 474)
VH FR4: WGKGTTVTVSS.

The four FRs of the ABTx070 antibody VL region are as follows:

(SEQ ID NO: 475)
VL FR1: DIVMTQSPSFLSASVGDRVTITCRAS;
(SEQ ID NO: 476)
VL FR2: LNWYQQKPGKAPKLLIY;
(SEQ ID NO: 477)
VL FR3: TLQNGVPSRFSGSGSGTDFTLTISSLQPEDFATYYC;
and
(SEQ ID NO: 478)
VL FR4: FGQGTRLEIKRTV.

By “ABTx070 polynucleotide” is meant a nucleic acid molecule (e.g., DNA) encoding at least a fragment of an ABTx070 antibody. In an embodiment, the encoded fragment has antigen binding activity.

By “ABTx071” is meant an antibody having at least about 85% amino acid sequence identity to an antibody sequence of antibody ABTx071 or comprising VH and/or VL CDRs 1-3 of ABTx071 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx071. Exemplary heavy chain and light chain sequences for antibody ABTx071 are provided below, where the variable regions are in plain text, the constant domains are in bold, and complementarity determining regions (CDRs), i.e., CDR1, CDR2, and CDR2, are underlined:

ABTx071 heavy chain (HC):
(SEQ ID NO: 479)
EVQLVESGGGVVRPGGSLRLSCAASGFTFDDYGMSWVRQAPGKGLEWVSG
INWNGGSTGYADSVKGRFTISRDNAKNSLHLQMNSLRAEDTALYYCARES
WNYEGYYYMDVWGKGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK
ABTx071 light chain (LC):
(SEQ ID NO: 480)
AIQMTQSPSFLSASVGDRVTITCRASQGISSYLAWYQQKPGKAPKLLIYA
ASTLQNGVPSRFSGSGSGTEFTLTISSLQPEDFATYYCQQLNSYPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The three CDRs of the ABTx071 antibody VH region are as follows:

(SEQ ID NO: 481)
VH CDR1: GFTFDDYG;
(SEQ ID NO: 482)
VH CDR2: INWNGGST
or
(SEQ ID NO: 1114)
INWNGGSTG;
and
(SEQ ID NO: 483)
VH CDR3: ARESWNYEGYYYMDV.

The three CDRs of the ABTx071 antibody VL region are as follows:

(SEQ ID NO: 484)
VL CDR1: QGISSY;
VL CDR2: AAS;
and
(SEQ ID NO: 485)
VL CDR3: QQLNSYPYT.

The four framework (FR) regions, i.e., FR1, FR2, FR3, and FR4, of the ABTx071 antibody are located on either side of each of the CDRs in VH and VL region sequences shown supra. In particular, the four FRs of the ABTx071 antibody VH region are as follows:

(SEQ ID NO: 486)
VH FR1: EVQLVESGGGVVRPGGSLRLSCAAS;
(SEQ ID NO: 487)
VH FR2: MSWVRQAPGKGLEWVSG;
(SEQ ID NO: 488)
VH FR3: GYADSVKGRFTISRDNAKNSLHLQMNSLRAEDTALYYC
or
(SEQ ID NO: 1117)
YADSVKGRFTISRDNAKNSLHLQMNSLRAEDTALYYC;
and
(SEQ ID NO: 489)
VH FR4: WGKGTTVTVSS.

The four FRs of the ABTx071 antibody VL region are as follows:

(SEQ ID NO: 490)
VL FR1: AIQMTQSPSFLSASVGDRVTITCRAS;
(SEQ ID NO: 491)
VL FR2: LAWYQQKPGKAPKLLIY;
(SEQ ID NO: 492)
VL FR3: TLQNGVPSRFSGSGSGTEFTLTISSLQPEDFATYYC;
and
(SEQ ID NO: 493)
VL FR4: FGQGTKLEIKRTV.

By “ABTx071 polynucleotide” is meant a nucleic acid molecule (e.g., DNA) encoding at least a fragment of an ABTx071 antibody. In an embodiment, the encoded fragment has antigen binding activity.

By “ABTx248” is meant an antibody having at least about 85% amino acid sequence identity to an antibody sequence of antibody ABTx248 or comprising VH and/or VL CDRs 1-3 of ABTx248 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx248. Exemplary heavy chain and light chain sequences for antibody ABTx248 are provided below, where the variable regions are in plain text, the constant domains are in bold, and complementarity determining regions (CDRs), i.e., CDR1, CDR2, and CDR2, are underlined:

ABTx248 heavy chain (HC):
(SEQ ID NO: 1011)
QVQLVQSGAAVKKPGESLKISCKGSGYRFTSYWIGWVRQMPGKGLEWMGI
IYPGDSDTRYSPSFQGQVTISAGKSISTAYLQWSSLKASDTAMYYCARHG
RGYDGYDGAFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PG
ABTx248 light chain (LC):
(SEQ ID NO: 420)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The three CDRs of the ABTx248 antibody VH region are as follows:

(SEQ ID NO: 421)
VH CDR1: GYRFTSYW;
(SEQ ID NO: 958)
VH CDR2: IYPGDSDTR;
and
(SEQ ID NO: 1012)
VH CDR3: ARHGRGYDGYDGAFDI.

The three CDRs of the ABTx248 antibody VL region are as follows:

(SEQ ID NO: 424)
VL CDR1: QGISSA;
VL CDR2: DAS;
and
(SEQ ID NO: 425)
VL CDR3: QQFNSYPLT.

The four framework (FR) regions, i.e., FR1, FR2, FR3, and FR4, of the ABTx248 antibody are located on either side of each of the CDRs in VH and VL region sequences shown supra. In particular, the four FRs of the ABTx248 antibody VH region are as follows:

(SEQ ID NO: 426)
VH FR1: QVQLVQSGAAVKKPGESLKISCKGS;
(SEQ ID NO: 427)
VH FR2: IGWVRQMPGKGLEWMGI;
and
(SEQ ID NO: 428)
VH FR3: RYSPSFQGQVTISAGKSISTAYLQWSSLKASDTAMYYC
or
(SEQ ID NO: 973)
YSPSFQGQVTISAGKSISTAYLQWSSLKASDTAMYYC;
(SEQ ID NO: 429)
VH FR4: WGQGTMVTVSS.

The four FRs of the ABTx248 antibody VL region are as follows:

(SEQ ID NO: 430)
VL FR1: AIQLTQSPSSLSASVGDRVTITCRAS;
(SEQ ID NO: 431)
VL FR2: LAWYQQKPGKAPKLLIY;
(SEQ ID NO: 432)
VL FR3: SLESGVPSRFSGSGSGTDFTLTISSLQPEDFATYYC;
and
(SEQ ID NO: 433)
VL FR4: FGGGTKVEIKRTV.

By “ABTx248 polynucleotide” is meant a nucleic acid molecule (e.g., DNA) encoding at least a fragment of an ABTx248 antibody. In an embodiment, the encoded fragment has antigen binding activity.

By “ABTx249” is meant an antibody having at least about 85% amino acid sequence identity to an antibody sequence of antibody ABTx249 or comprising VH and/or VL CDRs 1-3 of ABTx249 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx249. Exemplary heavy chain and light chain sequences for antibody ABTx249 are provided below, where the variable regions are in plain text, the constant domains are in bold, and complementarity determining regions (CDRs), i.e., CDR1, CDR2, and CDR2, are underlined:

ABTx249 heavy chain (HC):
(SEQ ID NO: 1013)
QVQLVQSGAAVKKPGESLKISCKGSGYRFTSYWIGWVRQMPGKGLEWMGI
IYPGDSDTRYSPSFQGQVTISAGKSISTAYLQWSSLKASDTAMYYCARHG
RGYDGYEGAFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PG
ABTx249 light chain (LC):
(SEQ ID NO: 1014)
AIQLTQSPSSLSASVGDRVTITCRASQGISSALAWYQQKPGKAPKLLIYD
ASSLESGVPSRFSGSGSGTDFTLTISSLQPEDFATYYCQQLNGYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The three CDRs of the ABTx249 antibody VH region are as follows:

VH CDR1:
(SEQ ID NO: 421)
GYRFTSYW;
VH CDR2:
(SEQ ID NO: 958)
IYPGDSDTR;
and
VH CDR3:
(SEQ ID NO: 948)
ARHGRGYDGYEGAFDI.

The three CDRs of the ABTx249 antibody VL region are as follows:

VL CDR1:
(SEQ ID NO: 424)
QGISSA;
VL CDR2:
DAS;
and
VL CDR3:
(SEQ ID NO: 1015)
QQLNGYPLT.

The four framework (FR) regions, i.e., FR1, FR2, FR3, and FR4, of the ABTx249 antibody are located on either side of each of the CDRs in VH and VL region sequences shown supra. In particular, the four FRs of the ABTx249 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx249 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx249 polynucleotide” is meant a nucleic acid molecule (e.g., DNA) encoding at least a fragment of an ABTx249 antibody. In an embodiment, the encoded fragment has antigen binding activity.

By “ABTx250” is meant an antibody having at least about 85% amino acid sequence identity to an antibody sequence of antibody ABTx250 or comprising VH and/or VL CDRs 1-3 of ABTx250 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx250. Exemplary heavy chain and light chain sequences for antibody ABTx250 are provided below, where the variable regions are in plain text, the constant domains are in bold, and complementarity determining regions (CDRs), i.e., CDR1, CDR2, and CDR2, are underlined:

ABTx250 heavy chain (HC):
(SEQ ID NO: 1016)
QVQLVQSGAAVKKPGESLKISCKGSGYRFTSYWIGWVRQMPGKGL
EWMGIIYPGDSDTKYSPSFQGQVTISAGKSISTAYLQWSSLKASD
TAMYYCARHGRGYDGYEGAFDIWGQGTMVTVSSASTKGPSVFPLA
PSSKSTSGGTAALGCLVKDYFPEPVTVSWNSGALTSGVHTFPAVL
QPSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PG
ABTx250 light chain (LC):
(SEQ ID NO: 420)
AIQLTQSPSSLSASVGDRVTITCRASQGISSALAWYQQKPGKAPK
LLIYDASSLESGVPSRFSGSGSGTDFTLTISSLQPEDFATYYCQQ
FNSYPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

The three CDRs of the ABTx250 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 948)
ARHGRGYDGYEGAFDI.
The three CDRs of the ABTx250 antibody
VL region are as follows:
VL CDR1:
(SEQ ID NO: 424)
QGISSA;
VL CDR2:
DAS;
and
VL CDR3:
(SEQ ID NO: 425)
QQFNSYPLT.

The four framework (FR) regions, i.e., FR1, FR2, FR3, and FR4, of the ABTx250 antibody are located on either side of each of the CDRs in VH and VL region sequences shown supra. In particular, the four FRs of the ABTx250 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx250 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx250 polynucleotide” is meant a nucleic acid molecule (e.g., DNA) encoding at least a fragment of an ABTx250 antibody. In an embodiment, the encoded fragment has antigen binding activity.

By “ABTx251” is meant an antibody having at least about 85% amino acid sequence identity to an antibody sequence of antibody ABTx251 or comprising VH and/or VL CDRs 1-3 of ABTx251 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx251. Exemplary heavy chain and light chain sequences for antibody ABTx251 are provided below, where the variable regions are in plain text, the constant domains are in bold, and complementarity determining regions (CDRs), i.e., CDR1, CDR2, and CDR2, are underlined:

ABTx251 heavy chain (HC):
(SEQ ID NO: 1013)
QVQLVQSGAAVKKPGESLKISCKGSGYRFTSYWIGWVRQMPGKGL
EWMGIIYPGDSDTRYSPSFQGQVTISAGKSISTAYLQWSSLKASD
TAMYYCARHGRGYDGYEGAFDIWGQGTMVTVSSASTKGPSVFPLA
PSSKSTSGGTAALGCLVKDYFPEPVTVSWNSGALTSGVHTFPAVL
QPSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PG
ABTx251 light chain (LC):
(SEQ ID NO: 1017)
AIQLTQSPSSLSASVGDRVTITCRASQGISSALAWYQQKPGKAPK
LLIYDASSLESGVPSRFSGSGSGTDFTLTISSLQPEDFATYYCQQ
FYSYPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

The three CDRs of the ABTx251 antibody VH region are as follows:

VH CDR1:
(SEQ ID NO: 421)
GYRFTSYW;
VH CDR2:
(SEQ ID NO: 958)
IYPGDSDTR;
and
VH CDR3:
(SEQ ID NO: 948)
ARHGRGYDGYEGAFDI.

The three CDRs of the ABTx251 antibody VL region are as follows:

VL CDR1:
(SEQ ID NO: 424)
QGISSA;
VL CDR2:
DAS;
and
VL CDR3:
(SEQ ID NO: 954)
QQFYSYPLT.

The four framework (FR) regions, i.e., FR1, FR2, FR3, and FR4, of the ABTx251 antibody are located on either side of each of the CDRs in VH and VL region sequences shown supra. In particular, the four FRs of the ABTx251 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx251 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx251 polynucleotide” is meant a nucleic acid molecule (e.g., DNA) encoding at least a fragment of an ABTx251 antibody. In an embodiment, the encoded fragment has antigen binding activity.

By “ABTx252” is meant an antibody having at least about 85% amino acid sequence identity to an antibody sequence of antibody ABTx252 or comprising VH and/or VL CDRs 1-3 of ABTx252 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx252. Exemplary heavy chain and light chain sequences for antibody ABTx252 are provided below, where the variable regions are in plain text, the constant domains are in bold, and complementarity determining regions (CDRs), i.e., CDR1, CDR2, and CDR2, are underlined:

ABTx252 heavy chain (HC):
(SEQ ID NO: 1013)
QVQLVQSGAAVKKPGESLKISCKGSGYRFTSYWIGWVRQMPGKGL
EWMGIIYPGDSDTRYSPSFQGQVTISAGKSISTAYLQWSSLKASD
TAMYYCARHGRGYDGYEGAFDIWGQGTMVTVSSASTKGPSVFPLA
PSSKSTSGGTAALGCLVKDYFPEPVTVSWNSGALTSGVHTFPAVL
QPSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PG
ABTx252 light chain (LC):
(SEQ ID NO: 1018)
AIQLTQSPSSLSASVGDRVTITCRASQGISSALAWYQQKPGKAPK
LLIYDASSLESGVPSRFSGSGSGTDFTLTISSLQPEDFATYYCQQ
FNSHPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

The three CDRs of the ABTx252 antibody VH region are as follows:

VH CDR1:
(SEQ ID NO: 421)
GYRFTSYW;
VH CDR2:
(SEQ ID NO: 958)
IYPGDSDTR;
and
VH CDR3:
(SEQ ID NO: 948)
ARHGRGYDGYEGAFDI.
The three CDRs of the ABTx252
antibody VL region are as follows:
VL CDR1:
(SEQ ID NO: 424)
QGISSA;
VL CDR2:
DAS;
and
VL CDR3:
(SEQ ID NO: 955)
QQFNSHPLT.

The four framework (FR) regions, i.e., FR1, FR2, FR3, and FR4, of the ABTx252 antibody are located on either side of each of the CDRs in VH and VL region sequences shown supra. In particular, the four FRs of the ABTx252 antibody VH region are as follows:

```
  (SEQ ID NO: 426)
VH FR1: QVQLVQSGAAVKKPGESLKISCKGS;
  (SEQ ID NO: 427)
VH FR2: IGWVRQMPGKGLEWMGI;
and
  (SEQ ID NO: 428)
VH FR3: RYSPSFQGQVTISAGKSISTAYLQWSSLKASDTAMYYC
or
  (SEQ ID NO: 973)
YSPSFQGQVTISAGKSISTAYLQWSSLKASDTAMYYC;
(SEQ ID NO: 429)
VH FR4: WGQGTMVTVSS
```

The four FRs of the ABTx252 antibody VL region are as follows:

```
  (SEQ ID NO: 430)
VL FR1: AIQLTQSPSSLSASVGDRVTITCRAS;
  (SEQ ID NO: 431)
VL FR2: LAWYQQKPGKAPKLLIY;
  (SEQ ID NO: 432)
VL FR3: SLESGVPSRFSGSGSGTDFTLTISSLQPEDFATYYC;
and
(SEQ ID NO: 433)
VL FR4: FGGGTKVEIKRTV.
```

By “ABTx252 polynucleotide” is meant a nucleic acid molecule (e.g., DNA) encoding at least a fragment of an ABTx252 antibody. In an embodiment, the encoded fragment has antigen binding activity.

By “ABTx253” is meant an antibody having at least about 85% amino acid sequence identity to an antibody sequence of antibody ABTx253 or comprising VH and/or VL CDRs 1-3 of ABTx253 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx253. Exemplary heavy chain and light chain sequences for antibody ABTx253 are provided below, where the variable regions are in plain text, the constant domains are in bold, and complementarity determining regions (CDRs), i.e., CDR1, CDR2, and CDR2, are underlined:

```
ABTx253 heavy chain (HC):
  (SEQ ID NO: 1013)
QVQLVQSGAAVKKPGESLKISCKGSGYRFTSYWIGWVRQMPGKGLEWMGIIYPGDSDTRYSPSF
QGQVTISAGKSISTAYLQWSSLKASDTAMYYCARHGRGYDGYEGAFDIWGQGTMVTVSSASTKG
PSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTOKSLS
LSPG
ABTx253 light chain (LC):
  (SEQ ID NO: 1019)
AIQLTQSPSSLSASVGDRVTITCRASQGISSALAWYQQKPGKAPKLLIYDASSLESGVPSRFSG
SGSGTDFTLTISSLQEDFATYYCQQFSSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC.
```

The three CDRs of the ABTx253 antibody VH region are as follows:

```
  (SEQ ID NO: 421)
VH CDR1: GYRFTSYW;
  (SEQ ID NO: 958)
VH CDR2: IYPGDSDTR;
and
(SEQ ID NO: 948)
VH CDR3: ARHGRGYDGYEGAFDI.
```

The three CDRs of the ABTx253 antibody VL region are as follows:

(SEQ ID NO: 424)
VL CDR1: QGISSA;
VL CDR2: DAS;
and
(SEQ ID NO: 1020)
VL CDR3: QQFSSYPLT.

The four framework (FR) regions, i.e., FR1, FR2, FR3, and FR4, of the ABTx253 antibody are located on either side of each of the CDRs in VH and VL region sequences shown supra. In particular, the four FRs of the ABTx253 antibody VH region are as follows:

```
  (SEQ ID NO: 426)
VH FR1: QVQLVQSGAAVKKPGESLKISCKGS;
  (SEQ ID NO: 427)
VH FR2: IGWVRQMPGKGLEWMGI;
and
  (SEQ ID NO: 428)
VH FR3: RYSPSFQGQVTISAGKSISTAYLQWSSLKASDTAMYYC
or
  (SEQ ID NO: 973)
YSPSFQGQVTISAGKSISTAYLQWSSLKASDTAMYYC;
(SEQ ID NO: 429)
VH FR4: WGQGTMVTVSS.
```

The four FRs of the ABTx253 antibody VL region are as follows:

```
  (SEQ ID NO: 430)
VL FR1: AIQLTQSPSSLSASVGDRVTITCRAS;
  (SEQ ID NO: 431)
VL FR2: LAWYQQKPGKAPKLLIY;
  (SEQ ID NO: 432)
VL FR3: SLESGVPSRFSGSGSGTDFTLTISSLOPEDFATYYC;
and
(SEQ ID NO: 433)
VL FR4: FGGGTKVEIKRTV.
```

By “ABTx253 polynucleotide” is meant a nucleic acid molecule (e.g., DNA) encoding at least a fragment of an ABTx253 antibody. In an embodiment, the encoded fragment has antigen binding activity.

By “ABTx254” is meant an antibody having at least about 85% amino acid sequence identity to an antibody sequence of antibody ABTx254 or comprising VH and/or VL CDRs 1-3 of ABTx254 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx254. Exemplary heavy chain and light chain sequences for antibody ABTx254 are provided below, where the variable regions are in plain text, the constant domains are in bold, and complementarity determining regions (CDRs), i.e., CDR1, CDR2, and CDR2, are underlined:

ABTx254 heavy chain (HC):
(SEQ ID NO: 1021)
QVQLVQSGAAVKKPGESLKISCKGSGYRFTSYWIGWVRQMPGKGLEWMGIIYPGDSDTKYSPSF
QGQVTISAGKSISTAYLQWSSLKASDTAMYYCARHGRGYDGYDGAFDIWGQGTMVTVSSASTKG
PSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSLS
LSPG
ABTx254 light chain (LC):
(SEQ ID NO: 1022)
AIQLTQSPSSLSASVGDRVTITCRASQGISSALAWYQQKPGKAPKLLIYDASSLESGVPSRFSG
SGSGTDFTLTISSLQPEDFATYYCQQTNSHPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSENRGEC.

The three CDRs of the ABTx254 antibody VH region are as follows:

```
  (SEQ ID NO: 421)
VH CDR1: GYRFTSYW;
  (SEQ ID NO: 972)
VH CDR2: IYPGDSDTK;
and
(SEQ ID NO: 1012)
VH CDR3: ARHGRGYDGYDGAFDI
```

The three CDRs of the ABTx254 antibody VL region are as follows:

```
  (SEQ ID NO: 424)
VL CDR1: QGISSA;
VL CDR2: DAS;
and
  (SEQ ID NO: 1023)
VL CDR3: QQTNSHPLT.
```

The four framework (FR) regions, i.e., FR1, FR2, FR3, and FR4, of the ABTx254 antibody are located on either side of each of the CDRs in VH and VL region sequences shown supra. In particular, the four FRs of the ABTx254 antibody VH region are as follows:

```
  (SEQ ID NO: 426)
VH FR1: QVQLVQSGAAVKKPGESLKISCKGS;
  (SEQ ID NO: 427)
VH FR2: IGWVRQMPGKGLEWMGI;
and
  (SEQ ID NO: 428)
VH FR3: RYSPSFQGQVTISAGKSISTAYLQWSSLKASDTAMYYC
or
  (SEQ ID NO: 973)
YSPSFQGQVTISAGKSISTAYLQWSSLKASDTAMYYC;
(SEQ ID NO: 429)
VH FR4: WGQGTMVTVSS.
```

The four FRs of the ABTx254 antibody VL region are as follows:

(SEQ ID NO: 430)
VL FR1: AIQLTQSPSSLSASVGDRVTITCRAS;
(SEQ ID NO: 431)
VL FR2: LAWYQQKPGKAPKLLIY;
(SEQ ID NO: 432)
VL FR3: SLESGVPSRFSGSGSGTDFTLTISSLQPEDFATYYC;
and
(SEQ ID NO: 433)
VL FR4: FGGGTKVEIKRTV.

By “ABTx254 polynucleotide” is meant a nucleic acid molecule (e.g., DNA) encoding at least a fragment of an ABTx254 antibody. In an embodiment, the encoded fragment has antigen binding activity.

By “ABTx255” is meant an antibody having at least about 85% amino acid sequence identity to an antibody sequence of antibody ABTx255 or comprising VH and/or VL CDRs 1-3 of ABTx255 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx255. Exemplary heavy chain and light chain sequences for antibody ABTx255 are provided below, where the variable regions are in plain text, the constant domains are in bold, and complementarity determining regions (CDRs), i.e., CDR1, CDR2, and CDR2, are underlined:

ABTx255 heavy chain (HC):
(SEQ ID NO: 1016)
QVQLVQSGAAVKKPGESLKISCKGSGYRFTSYWIGWVRQMPGKGLEWMGIIYPGDSDTKYSPSF
QGQVTISAGKSISTAYLQWSSLKASDTAMYYCARHGRGYDGYEGAFDIWGQGTMVTVSSASTKG
PSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSLS
LSPG
ABTx255 light chain (LC):
(SEQ ID NO: 1024)
AIQLTQSPSSLSASVGDRVTITCRASQGISSALAWYQQKPGKAPKLLIYDASSLESGVPSRFSG
SGSGTDFTLTISSLQPEDFATYYCQQVRSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC.

The three CDRs of the ABTx255 antibody VH region are as follows:

```
  (SEQ ID NO: 421)
VH CDR1: GYRFTSYW;
  (SEQ ID NO: 972)
VH CDR2: IYPGDSDTK; and
  (SEQ ID NO: 948)
VH CDR3: ARHGRGYDGYEGAFDI.
The three CDRs of the ABTx255 antibody VL
region are as follows:
  (SEQ ID NO: 424)
VL CDR1: QGISSA;
VL CDR2: DAS;
and
(SEQ ID NO: 1025)
VL CDR3: QQVRSYPLT.
```

The four framework (FR) regions, i.e., FR1, FR2, FR3, and FR4, of the ABTx255 antibody are located on either side of each of the CDRs in VH and VL region sequences shown supra. In particular, the four FRs of the ABTx255 antibody VH region are as follows:

```
  (SEQ ID NO: 426)
VH FR1: QVQLVQSGAAVKKPGESLKISCKGS;
  (SEQ ID NO: 427)
VH FR2: IGWVRQMPGKGLEWMGI;
and
  (SEQ ID NO: 428)
VH FR3: RYSPSFQGQVTISAGKSISTAYLQWSSLKASDTAMYYC
or
  (SEQ ID NO: 973)
YSPSFQGQVTISAGKSISTAYLQWSSLKASDTAMYYC;
(SEQ ID NO: 429)
VH FR4: WGQGTMVTVSS
```

The four FRs of the ABTx255 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx255 polynucleotide” is meant a nucleic acid molecule (e.g., DNA) encoding at least a fragment of an ABTx255 antibody. In an embodiment, the encoded fragment has antigen binding activity.

By “ABTx256” is meant an antibody having at least about 85% amino acid sequence identity to an antibody sequence of antibody ABTx256 or comprising VH and/or VL CDRs 1-3 of ABTx256 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx256. Exemplary heavy chain and light chain sequences for antibody ABTx256 are provided below, where the variable regions are in plain text, the constant domains are in bold, and complementarity determining regions (CDRs), i.e., CDR1, CDR2, and CDR2, are underlined:

ABTx256 heavy chain (HC):
(SEQ ID NO: 1026)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYDAYEGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx256 light chain (LC):
(SEQ ID NO: 1028)
AIQLTQSPSSLSASVGDRVTITCRASQGISSALAWYQQKPGKAPKLLIY
DASSLESGVPSRFSGSGSGTDFTLTISSLQPEDFATYYCQQTYSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx256 antibody VH region are as follows:

VH CDR1:
(SEQ ID NO: 421)
GYRFTSYW;
VH CDR2:
(SEQ ID NO: 958)
IYPGDSDTR;
and
VH CDR3:
(SEQ ID NO: 1027)
ARHGRGYDAYEGAFDI.

The three CDRs of the ABTx256 antibody VL region are as follows:

VL CDR1:
(SEQ ID NO: 424)
QGISSA;
VL CDR2:
DAS;
and
VL CDR3:
(SEQ ID NO: 1029)
QQTYSYPLT.

The four framework (FR) regions, i.e., FR1, FR2, FR3, and FR4, of the ABTx256 antibody are located on either side of each of the CDRs in VH and VL region sequences shown supra. In particular, the four FRs of the ABTx256 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx256 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx256 polynucleotide” is meant a nucleic acid molecule (e.g., DNA) encoding at least a fragment of an ABTx256 antibody. In an embodiment, the encoded fragment has antigen binding activity.

By “ABTx257” is meant an antibody having at least about 85% amino acid sequence identity to an antibody sequence of antibody ABTx257 or comprising VH and/or VL CDRs 1-3 of ABTx257 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx257. Exemplary heavy chain and light chain sequences for antibody ABTx257 are provided below, where the variable regions are in plain text, the constant domains are in bold, and complementarity determining regions (CDRs), i.e., CDR1, CDR2, and CDR2, are underlined:

ABTx257 heavy chain (HC):
(SEQ ID NO: 1030)
QVQLVQSGAAVKKPGESLKISCKGSGYRFTSYWIGWVRQMPGKGLEWMG
IIYPGDSDTKYSPSFQGQVTISAGKSISTAYLQWSSLKASDTAMYYCAR
HGRGYDAYDGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx257 light chain (LC):
(SEQ ID NO: 1031)
AIQLTQSPSSLSASVGDRVTITCRASQGISSALAWYQQKPGKAPKLLIY
DASSLESGVPSRFSGSGSGTDFTLTISSLQPEDFATYYCQQTNSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx257 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 1032)
ARHGRGYDAYDGAFDI.
The three CDRs of the ABTx257 antibody VL region
are as follows:
VL CDR1:
(SEQ ID NO: 424)
QGISSA;
VL CDR2:
DAS;
and
VL CDR3:
(SEQ ID NO: 1044)
QQTNSYPLT.

The four framework (FR) regions, i.e., FR1, FR2, FR3, and FR4, of the ABTx257 antibody are located on either side of each of the CDRs in VH and VL region sequences shown supra. In particular, the four FRs of the ABTx257 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx257 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx257 polynucleotide” is meant a nucleic acid molecule (e.g., DNA) encoding at least a fragment of an ABTx257 antibody. In an embodiment, the encoded fragment has antigen binding activity.

By “ABTx258” is meant an antibody having at least about 85% amino acid sequence identity to an antibody sequence of antibody ABTx258 or comprising VH and/or VL CDRs 1-3 of ABTx258 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx258. Exemplary heavy chain and light chain sequences for antibody ABTx258 are provided below, where the variable regions are in plain text, the constant domains are in bold, and complementarity determining regions (CDRs), i.e., CDR1, CDR2, and CDR2, are underlined:

ABTx258 heavy chain (HC):
(SEQ ID NO: 1034)
QVQLVQSGAAVKKPGESLKISCKGSGYRFTSYWIGWVRQMPGKGLEWMG
IIYPGDSDTKYSPSFQGQVTISAGKSISTAYLQWSSLKASDTAMYYCAR
HGRGYDAYEGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx258 light chain (LC):
(SEQ ID NO: 1024)
AIQLTQSPSSLSASVGDRVTITCRASQGISSALAWYQQKPGKAPKLLIY
DASSLESGVPSRFSGSGSGTDFTLTISSLQPEDFATYYCQQVRSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx258 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 1027)
ARHGRGYDAYEGAFDI.
The three CDRs of the ABTx058 antibody VL region
are as follows:
VL CDR1:
(SEQ ID NO: 424)
QGISSA;
VL CDR2:
DAS;
and
VL CDR3:
(SEQ ID NO: 1025)
QQVRSYPLT.

The four framework (FR) regions, i.e., FR1, FR2, FR3, and FR4, of the ABTx258 antibody are located on either side of each of the CDRs in VH and VL region sequences shown supra. In particular, the four FRs of the ABTx258 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx258 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx258 polynucleotide” is meant a nucleic acid molecule (e.g., DNA) encoding at least a fragment of an ABTx258 antibody. In an embodiment, the encoded fragment has antigen binding activity.

By “ABTx259” is meant an antibody having at least about 85% amino acid sequence identity to an antibody sequence of antibody ABTx259 or comprising VH and/or VL CDRs 1-3 of ABTx259 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx259. Exemplary heavy chain and light chain sequences for antibody ABTx259 are provided below, where the variable regions are in plain text, the constant domains are in bold, and complementarity determining regions (CDRs), i.e., CDR1, CDR2, and CDR2, are underlined:

ABTx259 heavy chain (HC):
(SEQ ID NO: 1016)
QVQLVQSGAAVKKPGESLKISCKGSGYRFTSYWIGWVRQMPGKGLEWMG
IIYPGDSDTKYSPSFQGQVTISAGKSISTAYLQWSSLKASDTAMYYCAR
HGRGYDGYEGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx259 light chain (LC):
(SEQ ID NO: 1035)
AIQLTQSPSSLSASVGDRVTITCRASQGISSALAWYQQKPGKAPKLLIY
DASSLESGVPSRFSGSGSGTDFTLTISSLQPEDFATYYCQQTRSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx259 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 948)
ARHGRGYDGYEGAFDI.
The three CDRs of the ABTx259 antibody VL region
are as follows:
VL CDR1:
(SEQ ID NO: 424)
QGISSA;
VL CDR2:
DAS;
and
VL CDR3:
(SEQ ID NO: 1036)
QQTRSYPLT.

The four framework (FR) regions, i.e., FR1, FR2, FR3, and FR4, of the ABTx259 antibody are located on either side of each of the CDRs in VH and VL region sequences shown supra. In particular, the four FRs of the ABTx259 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx259 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx259 polynucleotide” is meant a nucleic acid molecule (e.g., DNA) encoding at least a fragment of an ABTx259 antibody. In an embodiment, the encoded fragment has antigen binding activity.

By “ABTx260” is meant an antibody having at least about 85% amino acid sequence identity to an antibody sequence of antibody ABTx260 or comprising VH and/or VL CDRs 1-3 of ABTx260 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx260. Exemplary heavy chain and light chain sequences for antibody ABTx260 are provided below, where the variable regions are in plain text, the constant domains are in bold, and complementarity determining regions (CDRs), i.e., CDR1, CDR2, and CDR2, are underlined:

ABTx260 heavy chain (HC):
(SEQ ID NO: 1021)
QVQLVQSGAAVKKPGESLKISCKGSGYRFTSYWIGWVRQMPGKGLEWMG
IIYPGDSDTKYSPSFQGQVTISAGKSISTAYLQWSSLKASDTAMYYCAR
HGRGYDGYDGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx260 light chain (LC):
(SEQ ID NO: 1037)
AIQLTQSPSSLSASVGDRVTITCRASQGISSALAWYQQKPGKAPKLLIY
DASSLESGVPSRFSGSGSGTDFTLTISSLQPEDFATYYCQQVNSHPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx260 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 1012)
ARHGRGYDGYDGAFDI.

The three CDRs of the ABTx260 antibody VL region are as follows:

VL CDR1:
(SEQ ID NO: 424)
QGISSA;
VL CDR2:
DAS;
and
VL CDR3:
(SEQ ID NO: 1038)
QQVNSHPLT.

The four framework (FR) regions, i.e., FR1, FR2, FR3, and FR4, of the ABTx260 antibody are located on either side of each of the CDRs in VH and VL region sequences shown supra. In particular, the four FRs of the ABTx260 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx260 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx260 polynucleotide” is meant a nucleic acid molecule (e.g., DNA) encoding at least a fragment of an ABTx260 antibody. In an embodiment, the encoded fragment has antigen binding activity.

By “ABTx261” is meant an antibody having at least about 85% amino acid sequence identity to an antibody sequence of antibody ABTx261 or comprising VH and/or VL CDRs 1-3 of ABTx261 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx261. Exemplary heavy chain and light chain sequences for antibody ABTx261 are provided below, where the variable regions are in plain text, the constant domains are in bold, and complementarity determining regions (CDRs), i.e., CDR1, CDR2, and CDR2, are underlined:

ABTx261 heavy chain (HC):
(SEQ ID NO: 1034)
QVQLVQSGAAVKKPGESLKISCKGSGYRFTSYWIGWVRQMPGKGLEWMG
IIYPGDSDTKYSPSFQGQVTISAGKSISTAYLQWSSLKASDTAMYYCAR
HGRGYDAYEGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx261 light chain (LC):
(SEQ ID NO: 1035)
AIQLTQSPSSLSASVGDRVTITCRASQGISSALAWYQQKPGKAPKLLIY
DASSLESGVPSRFSGSGSGTDFTLTISSLQPEDFATYYCQQTRSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx261 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 1027)
ARHGRGYDAYEGAFDI.

The three CDRs of the ABTx261 antibody VL region are as follows:

VL CDR1:
(SEQ ID NO: 424)
QGISSA;
VL CDR2:
DAS;
and
VL CDR3:
(SEQ ID NO: 1036)
QQTRSYPLT.

The four framework (FR) regions, i.e., FR1, FR2, FR3, and FR4, of the ABTx261 antibody are located on either side of each of the CDRs in VH and VL region sequences shown supra. In particular, the four FRs of the ABTx261 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx261 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx261 polynucleotide” is meant a nucleic acid molecule (e.g., DNA) encoding at least a fragment of an ABTx261 antibody. In an embodiment, the encoded fragment has antigen binding activity.

By “ABTx262” is meant an antibody having at least about 85% amino acid sequence identity to an antibody sequence of antibody ABTx262 or comprising VH and/or VL CDRs 1-3 of ABTx262 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx262. Exemplary heavy chain and light chain sequences for antibody ABTx262 are provided below, where the variable regions are in plain text, the constant domains are in bold, and complementarity determining regions (CDRs), i.e., CDR1, CDR2, and CDR2, are underlined:

ABTx262 heavy chain (HC):
(SEQ ID NO: 1026)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYDAYEGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx262 light chain (LC):
(SEQ ID NO: 1039)
AIQLTQSPSSLSASVGDRVTITCRASQGISSALAWYQQKPGKAPKLLIY
DASSLESGVPSRFSGSGSGTDFTLTISSLQPEDFATYYCQQFRSSPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx262 antibody VH region are as follows:

VH CDR1:
(SEQ ID NO: 421)
GYRFTSYW;
VH CDR2:
(SEQ ID NO: 958)
IYPGDSDTR;
and
VH CDR3:
(SEQ ID NO: 1027)
ARHGRGYDAYEGAFDI.

The three CDRs of the ABTx262 antibody VL region are as follows:

(SEQ ID NO: 424)
VL CDR1: QGISSA;
VL CDR2: DAS;
and
(SEQ ID NO: 1040)
VL CDR3: QQFRSSPLT.

The four framework (FR) regions, i.e., FR1, FR2, FR3, and FR4, of the ABTx262 antibody are located on either side of each of the CDRs in VH and VL region sequences shown supra. In particular, the four FRs of the ABTx262 antibody VH region are as follows:

(SEQ ID NO: 426)
VH FR1: QVQLVQSGAAVKKPGESLKISCKGS;
(SEQ ID NO: 427)
VH FR2: IGWVRQMPGKGLEWMGI;
and
(SEQ ID NO: 428)
VH FR3: RYSPSFQGQVTISAGKSISTAYLQWSSLKASDTAMYYC
or
(SEQ ID NO: 973)
YSPSFQGQVTISAGKSISTAYLQWSSLKASDTAMYYC;
(SEQ ID NO: 429)
VH FR4: WGQGTMVTVSS.

The four FRs of the ABTx262 antibody VL region are as follows:

(SEQ ID NO: 430)
VL FR1: AIQLTQSPSSLSASVGDRVTITCRAS;
(SEQ ID NO: 431)
VL FR2: LAWYQQKPGKAPKLLIY;
(SEQ ID NO: 432)
VL FR3: SLESGVPSRFSGSGSGTDFTLTISSLQPEDFATYYC;
and
(SEQ ID NO: 433)
VL FR4: FGGGTKVEIKRTV.

By “ABTx262 polynucleotide” is meant a nucleic acid molecule (e.g., DNA) encoding at least a fragment of an ABTx262 antibody. In an embodiment, the encoded fragment has antigen binding activity.

By “ABTx263” is meant an antibody having at least about 85% amino acid sequence identity to an antibody sequence of antibody ABTx263 or comprising VH and/or VL CDRs 1-3 of ABTx263 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx263. Exemplary heavy chain and light chain sequences for antibody ABTx263 are provided below, where the variable regions are in plain text, the constant domains are in bold, and complementarity determining regions (CDRs), i.e., CDR1, CDR2, and CDR2, are underlined:

ABTx263 heavy chain (HC):
(SEQ ID NO: 1021)
QVQLVQSGAAVKKPGESLKISCKGSGYRFTSYWIGWVRQMPGKGLEWMGI
IYPGDSDTKYSPSFQGQVTISAGKSISTAYLQWSSLKASDTAMYYCARHG
RGYDGYDGAFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PG
ABTx263 light chain (LC):
(SEQ ID NO: 1041)
AIQLTQSPSSLSASVGDRVTITCRASQGISSALAWYQQKPGKAPKLLIYD
ASSLESGVPSRFSGSGSGTDFTLTISSLQPEDFATYYCQQVYSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The three CDRs of the ABTx263 antibody VH region are as follows:

(SEQ ID NO: 421)
VH CDR1: GYRFTSYW;
(SEQ ID NO: 972)
VH CDR2: IYPGDSDTK;
and
(SEQ ID NO: 1012)
VH CDR3: ARHGRGYDGYDGAFDI.

The three CDRs of the ABTx263 antibody VL region are as follows:

(SEQ ID NO: 424)
VL CDR1: QGISSA;
VL CDR2: DAS;
and
(SEQ ID NO: 1042)
VL CDR3: QQVYSYPLT.

The four framework (FR) regions, i.e., FR1, FR2, FR3, and FR4, of the ABTx263 antibody are located on either side of each of the CDRs in VH and VL region sequences shown supra. In particular, the four FRs of the ABTx263 antibody VH region are as follows:

(SEQ ID NO: 426)
VH FR1: QVQLVQSGAAVKKPGESLKISCKGS;
(SEQ ID NO: 427)
VH FR2: IGWVRQMPGKGLEWMGI;
and
(SEQ ID NO: 428)
VH FR3: RYSPSFQGQVTISAGKSISTAYLQWSSLKASDTAMYYC
or
(SEQ ID NO: 973)
YSPSFQGQVTISAGKSISTAYLQWSSLKASDTAMYYC;
(SEQ ID NO: 429)
VH FR4: WGQGTMVTVSS.

The four FRs of the ABTx263 antibody VL region are as follows:

(SEQ ID NO: 430)
VL FR1: AIQLTQSPSSLSASVGDRVTITCRAS;
(SEQ ID NO: 431)
VL FR2: LAWYQQKPGKAPKLLIY;
(SEQ ID NO: 432)
VL FR3: SLESGVPSRFSGSGSGTDFTLTISSLQPEDFATYYC;
and
(SEQ ID NO: 433)
VL FR4: FGGGTKVEIKRTV.

By “ABTx263 polynucleotide” is meant a nucleic acid molecule (e.g., DNA) encoding at least a fragment of an ABTx263 antibody. In an embodiment, the encoded fragment has antigen binding activity.

By “ABTx264” is meant an antibody having at least about 85% amino acid sequence identity to an antibody sequence of antibody ABTx264 or comprising VH and/or VL CDRs 1-3 of ABTx264 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx264. Exemplary heavy chain and light chain sequences for antibody ABTx264 are provided below, where the variable regions are in plain text, the constant domains are in bold, and complementarity determining regions (CDRs), i.e., CDR1, CDR2, and CDR2, are underlined:

ABTx264 heavy chain (HC):
(SEQ ID NO: 1034)
QVQLVQSGAAVKKPGESLKISCKGSGYRFTSYWIGWVRQMPGKGLEWMGI
IYPGDSDTKYSPSFQGQVTISAGKSISTAYLQWSSLKASDTAMYYCARHG
RGYDAYEGAFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PG
ABTx264 light chain (LC):
(SEQ ID NO: 1028)
AIQLTQSPSSLSASVGDRVTITCRASQGISSALAWYQQKPGKAPKLLIYD
ASSLESGVPSRFSGSGSGTDFTLTISSLQPEDFATYYCQQTYSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSENRGEC.

The three CDRs of the ABTx264 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 1027)
ARHGRGYDAYEGAFDI.

The three CDRs of the ABTx264 antibody VL region are as follows:

(SEQ ID NO: 424)
VL CDR1: QGISSA;
VL CDR2: DAS;
and
(SEQ ID NO: 1029)
VL CDR3: QQTYSYPLT.

The four framework (FR) regions, i.e., FR1, FR2, FR3, and FR4, of the ABTx264 antibody are located on either side of each of the CDRs in VH and VL region sequences shown supra. In particular, the four FRs of the ABTx264 antibody VH region are as follows:

(SEQ ID NO: 426)
VH FR1: QVQLVQSGAAVKKPGESLKISCKGS;
(SEQ ID NO: 427)
VH FR2: IGWVRQMPGKGLEWMGI;
and
(SEQ ID NO: 428)
VH FR3: RYSPSFQGQVTISAGKSISTAYLQWSSLKASDTAMYYC
or
(SEQ ID NO: 973)
YSPSFQGQVTISAGKSISTAYLQWSSLKASDTAMYYC;
(SEQ ID NO: 429)
VH FR4: WGQGTMVTVSS.

The four FRs of the ABTx264 antibody VL region are as follows:

(SEQ ID NO: 430)
VL FR1: AIQLTQSPSSLSASVGDRVTITCRAS;
(SEQ ID NO: 431)
VL FR2: LAWYQQKPGKAPKLLIY;
(SEQ ID NO: 432)
VL FR3: SLESGVPSRFSGSGSGTDFTLTISSLQPEDFATYYC;
and
(SEQ ID NO: 433)
VL FR4: FGGGTKVEIKRTV.

By “ABTx264 polynucleotide” is meant a nucleic acid molecule (e.g., DNA) encoding at least a fragment of an ABTx264 antibody. In an embodiment, the encoded fragment has antigen binding activity.

By “ABTx265” is meant an antibody having at least about 85% amino acid sequence identity to an antibody sequence of antibody ABTx265 or comprising VH and/or VL CDRs 1-3 of ABTx265 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx265. Exemplary heavy chain and light chain sequences for antibody ABTx265 are provided below, where the variable regions are in plain text, the constant domains are in bold, and complementarity determining regions (CDRs), i.e., CDR1, CDR2, and CDR2, are underlined:

ABTx265 heavy chain (HC):
(SEQ ID NO: 1021)
QVQLVQSGAAVKKPGESLKISCKGSGYRFTSYWIGWVRQMPGKGLEWMGI
IYPGDSDTKYSPSFQGQVTISAGKSISTAYLQWSSLKASDTAMYYCARHG
RGYDGYDGAFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PG
ABTx265 light chain (LC):
(SEQ ID NO: 1035)
AIQLTQSPSSLSASVGDRVTITCRASQGISSALAWYQQKPGKAPKLLIYD
ASSLESGVPSRFSGSGSGTDFTLTISSLQPEDFATYYCQQTRSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The three CDRs of the ABTx265 antibody VH region are as follows:

(SEQ ID NO: 421)
VH CDR1: GYRFTSYW;
(SEQ ID NO: 972)
VH CDR2: IYPGDSDTK;
and
(SEQ ID NO: 1012)
VH CDR3: ARHGRGYDGYDGAFDI.

The three CDRs of the ABTx265 antibody VL region are as follows:

(SEQ ID NO: 424)
VL CDR1: QGISSA;
VL CDR2: DAS;
and
(SEQ ID NO: 1036)
VL CDR3: QQTRSYPLT.

The four framework (FR) regions, i.e., FR1, FR2, FR3, and FR4, of the ABTx265 antibody are located on either side of each of the CDRs in VH and VL region sequences shown supra. In particular, the four FRs of the ABTx265 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx265 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx265 polynucleotide” is meant a nucleic acid molecule (e.g., DNA) encoding at least a fragment of an ABTx265 antibody. In an embodiment, the encoded fragment has antigen binding activity.

By “ABTx266” is meant an antibody having at least about 85% amino acid sequence identity to an antibody sequence of antibody ABTx266 or comprising VH and/or VL CDRs 1-3 of ABTx266 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx266. Exemplary heavy chain and light chain sequences for antibody ABTx266 are provided below, where the variable regions are in plain text, the constant domains are in bold, and complementarity determining regions (CDRs), i.e., CDR1, CDR2, and CDR2, are underlined:

ABTx266 heavy chain (HC):
(SEQ ID NO: 1026)
QVQLVQSGAAVKKPGESLKISCKGSGYRFTSYWIGWVRQMPGKGL
EWMGIIYPGDSDTRYSPSFQGQVTISAGKSISTAYLQWSSLKASD
TAMYYCARHGRGYDAYEGAFDIWGQGTMVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PG
ABTx266 light chain (LC):
(SEQ ID NO: 1046)
AIQLTQSPSSLSASVGDRVTITCRASQGISSALAWYQQKPGKAPK
LLIYDASSLESGVPSRFSGSGSGTDFTLTISSLQPEDFATYYCQQ
VRSHPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC.

The three CDRs of the ABTx266 antibody VH region are as follows:

VH CDR1:
(SEQ ID NO: 421)
GYRFTSYW;
VH CDR2:
(SEQ ID NO: 958)
IYPGDSDTR;
and
VH CDR3:
(SEQ ID NO: 1027)
ARHGRGYDAYEGAFDI.
The three CDRs of the ABTx266
antibody VL region are as follows:
VL CDR1:
(SEQ ID NO: 424)
QGISSA;
VL CDR2:
DAS;
and
VL CDR3:
(SEQ ID NO: 1047)
QQVRSHPLT.

The four framework (FR) regions, i.e., FR1, FR2, FR3, and FR4, of the ABTx266 antibody are located on either side of each of the CDRs in VH and VL region sequences shown supra. In particular, the four FRs of the ABTx266 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx266 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx266 polynucleotide” is meant a nucleic acid molecule (e.g., DNA) encoding at least a fragment of an ABTx266 antibody. In an embodiment, the encoded fragment has antigen binding activity.

By “ABTx267” is meant an antibody having at least about 85% amino acid sequence identity to an antibody sequence of antibody ABTx267 or comprising VH and/or VL CDRs 1-3 of ABTx267 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx267. Exemplary heavy chain and light chain sequences for antibody ABTx267 are provided below, where the variable regions are in plain text, the constant domains are in bold, and complementarity determining regions (CDRs), i.e., CDR1, CDR2, and CDR2, are underlined:

ABTx267 heavy chain (HC):
(SEQ ID NO: 1030)
QVQLVQSGAAVKKPGESLKISCKGSGYRFTSYWIGWVRQMPGKGL
EWMGIIYPGDSDTKYSPSFQGQVTISAGKSISTAYLQWSSLKASD
TAMYYCARHGRGYDAYDGAFDIWGQGTMVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PG
ABTx267 light chain (LC):
(SEQ ID NO: 1048)
AIQLTQSPSSLSASVGDRVTITCRASQGISSALAWYQQKPGKAPK
LLIYDASSLESGVPSRFSGSGSGTDFTLTISSLQPEDFATYYCQQ
TNSSPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

The three CDRs of the ABTx267 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 1032)
ARHGRGYDAYDGAFDI.

The three CDRs of the ABTx267 antibody VL region are as follows:

VL CDR1:
(SEQ ID NO: 424)
QGISSA;
VL CDR2:
DAS;
and
VL CDR3:
(SEQ ID NO: 1050)
QQTNSSPLT.

The four framework (FR) regions, i.e., FR1, FR2, FR3, and FR4, of the ABTx267 antibody are located on either side of each of the CDRs in VH and VL region sequences shown supra. In particular, the four FRs of the ABTx267 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx267 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx267 polynucleotide” is meant a nucleic acid molecule (e.g., DNA) encoding at least a fragment of an ABTx267 antibody. In an embodiment, the encoded fragment has antigen binding activity.

By “ABTx268” is meant an antibody having at least about 85% amino acid sequence identity to an antibody sequence of antibody ABTx268 or comprising VH and/or VL CDRs 1-3 of ABTx268 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx268. Exemplary heavy chain and light chain sequences for antibody ABTx268 are provided below, where the variable regions are in plain text, the constant domains are in bold, and complementarity determining regions (CDRs), i.e., CDR1, CDR2, and CDR2, are underlined:

ABTx268 heavy chain (HC):
(SEQ ID NO: 1016)
QVQLVQSGAAVKKPGESLKISCKGSGYRFTSYWIGWVRQMPGKGL
EWMGIIYPGDSDTKYSPSFQGQVTISAGKSISTAYLQWSSLKASD
TAMYYCARHGRGYDGYEGAFDIWGQGTMVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLS
LSPG
ABTx268 light chain (LC):
(SEQ ID NO: 1049)
AIQLTQSPSSLSASVGDRVTITCRASQGISSALAWYQQKPGKAPK
LLIYDASSLESGVPSRFSGSGSGTDFTLTISSLQPEDFATYYCQQ
VNSYPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC

The three CDRs of the ABTx268 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 948)
ARHGRGYDGYEGAFDI.

The three CDRs of the ABTx268 antibody VL region are as follows:

VL CDR1:
(SEQ ID NO: 424)
QGISSA;
VL CDR2:
DAS;
and
VL CDR3:
(SEQ ID NO: 952)
QQVNSYPLT.

The four framework (FR) regions, i.e., FR1, FR2, FR3, and FR4, of the ABTx268 antibody are located on either side of each of the CDRs in VH and VL region sequences shown supra. In particular, the four FRs of the ABTx268 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx268 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx268 polynucleotide” is meant a nucleic acid molecule (e.g., DNA) encoding at least a fragment of an ABTx268 antibody. In an embodiment, the encoded fragment has antigen binding activity.

By “ABTx269” is meant an antibody having at least about 85% amino acid sequence identity to an antibody sequence of antibody ABTx269 or comprising VH and/or VL CDRs 1-3 of ABTx269 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx269. Exemplary heavy chain and light chain sequences for antibody ABTx269 are provided below, where the variable regions are in plain text, the constant domains are in bold, and complementarity determining regions (CDRs), i.e., CDR1, CDR2, and CDR2, are underlined:

ABTx269 heavy chain (HC):
(SEQ ID NO: 1016)
QVQLVQSGAAVKKPGESLKISCKGSGYRFTSYWIGWVRQMPGKGLEWMG
IIYPGDSDTKYSPSFQGQVTISAGKSISTAYLQWSSLKASDTAMYYCAR
HGRGYDGYEGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx269 light chain (LC):
(SEQ ID NO: 1041)
AIQLTQSPSSLSASVGDRVTITCRASQGISSALAWYQQKPGKAPKLLIY
DASSLESGVPSRFSGSGSGTDFTLTISSLQPEDFATYYCQQVYSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx269 antibody VH region are as follows:

VH CDR1:
(SEQ ID NO: 421)
GYRFTSYW;
VH CDR2:
(SEQ ID NO: 972)
IYPGDSDTK;
and
VH CDR3:
(SEQ ID NO: 948)
ARHGRGYDGYEGAFDI.

The three CDRs of the ABTx269 antibody VL region are as follows:

VL CDR1:
(SEQ ID NO: 424)
QGISSA;
VL CDR2:
DAS;
and
VL CDR3:
(SEQ ID NO: 1042)
QQVYSYPLT.

The four framework (FR) regions, i.e., FR1, FR2, FR3, and FR4, of the ABTx269 antibody are located on either side of each of the CDRs in VH and VL region sequences shown supra. In particular, the four FRs of the ABTx269 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx269 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx269 polynucleotide” is meant a nucleic acid molecule (e.g., DNA) encoding at least a fragment of an ABTx269 antibody. In an embodiment, the encoded fragment has antigen binding activity.

By “ABTx270” is meant an antibody having at least about 85% amino acid sequence identity to an antibody sequence of antibody ABTx270 or comprising VH and/or VL CDRs 1-3 of ABTx270 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx270. Exemplary heavy chain and light chain sequences for antibody ABTx270 are provided below, where the variable regions are in plain text, the constant domains are in bold, and complementarity determining regions (CDRs), i.e., CDR1, CDR2, and CDR2, are underlined:

ABTx270 heavy chain (HC):
(SEQ ID NO: 1026)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYDAYEGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx270 light chain (LC):
(SEQ ID NO: 1024)
AIQLTQSPSSLSASVGDRVTITCRASQGISSALAWYQQKPGKAPKLLIY
DASSLESGVPSRFSGSGSGTDFTLTISSLQPEDFATYYCQQVRSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx270 antibody VH region are as follows:

VH CDR1:
(SEQ ID NO: 421)
GYRFTSYW;
VH CDR2:
(SEQ ID NO: 958)
IYPGDSDTR;
and
VH CDR3:
(SEQ ID NO: 1027)
ARHGRGYDAYEGAFDI.

The three CDRs of the ABTx270 antibody VL region are as follows:

VL CDR1:
(SEQ ID NO: 424)
QGISSA;
VL CDR2:
DAS;
and
VL CDR3:
(SEQ ID NO: 1025)
QQVRSYPLT.

The four framework (FR) regions, i.e., FR1, FR2, FR3, and FR4, of the ABTx270 antibody are located on either side of each of the CDRs in VH and VL region sequences shown supra. In particular, the four FRs of the ABTx270 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx270 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx270 polynucleotide” is meant a nucleic acid molecule (e.g., DNA) encoding at least a fragment of an ABTx270 antibody. In an embodiment, the encoded fragment has antigen binding activity.

By “ABTx271” is meant an antibody having at least about 85% amino acid sequence identity to an antibody sequence of antibody ABTx271 or comprising VH and/or VL CDRs 1-3 of ABTx271 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx271. Exemplary heavy chain and light chain sequences for antibody ABTx271 are provided below, where the variable regions are in plain text, the constant domains are in bold, and complementarity determining regions (CDRs), i.e., CDR1, CDR2, and CDR2, are underlined:

ABTx271 heavy chain (HC):
(SEQ ID NO: 1043)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYDAYDGAFDIWGQGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPG
ABTx271 light chain (LC):
(SEQ ID NO: 1031)
AIQLTQSPSSLSASVGDRVTITCRASQGISSALAWYQQKPGKAPKLLIY
DASSLESGVPSRFSGSGSGTDFTLTISSLQPEDFATYYCQQTNSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The three CDRs of the ABTx271 antibody VH region are as follows:

VH CDR1:
(SEQ ID NO: 421)
GYRFTSYW;
VH CDR2:
(SEQ ID NO: 958)
IYPGDSDTR;
and
VH CDR3:
(SEQ ID NO: 1032)
ARHGRGYDAYDGAFDI.
The three CDRs of the ABTx271 antibody VL region
are as follows:
VL CDR1:
(SEQ ID NO: 424)
QGISSA;
VL CDR2:
DAS;
and
VL CDR3:
(SEQ ID NO: 1044)
QQTNSYPLT.

The four framework (FR) regions, i.e., FR1, FR2, FR3, and FR4, of the ABTx271 antibody are located on either side of each of the CDRs in VH and VL region sequences shown supra. In particular, the four FRs of the ABTx271 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx271 antibody VL region are as follows:

VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 433)
FGGGTKVEIKRTV.

By “ABTx271 polynucleotide” is meant a nucleic acid molecule (e.g., DNA) encoding at least a fragment of an ABTx271 antibody. In an embodiment, the encoded fragment has antigen binding activity.

By “ABTx272” is meant an antibody having at least about 85% amino acid sequence identity to an antibody sequence of antibody ABTx272 or comprising VH and/or VL CDRs 1-3 of ABTx272 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx272. Exemplary heavy chain and light chain sequences for antibody ABTx272 are provided below, where the variable regions are in plain text, the constant domains are in bold, and complementarity determining regions (CDRs), i.e., CDR1, CDR2, and CDR2, are underlined:

ABTx272 heavy chain (HC):
(SEQ ID NO: 1021)
QVQLVQSGAAVKKPGESLKISCKGSGYRFTSYWIGWVRQMPGKGLEWMGIIYPGDSDTKYSPSF
QGQVTISAGKSISTAYLQWSSLKASDTAMYYCARHGRGYDGYDGAFDIWGQGTMVTVSSASTKG
PSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTOKSLS
LSPG
ABTx272 light chain (LC):
(SEQ ID NO: 1024)
AIQLTQSPSSLSASVGDRVTITCRASQGISSALAWYQQKPGKAPKLLIYDASSLESGVPSRFSG
SGSGTDFTLTISSLQPEDFATYYCQQVRSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSENRGEC

The three CDRs of the ABTx272 antibody VH region are as follows:

```
  (SEQ ID NO: 421)
VH CDR1: GYRFTSYW;
  (SEQ ID NO: 972)
VH CDR2: IYPGDSDTK;
and
(SEQ ID NO: 1012)
VH CDR3: ARHGRGYDGYDGAFDI.
```

The three CDRs of the ABTx272 antibody VL region are as follows:

(SEQ ID NO: 424)
VL CDR1: QGISSA;
VL CDR2: DAS;
and
(SEQ ID NO: 1025)
VL CDR3: QQVRSYPLT

The four framework (FR) regions, i.e., FR1, FR2, FR3, and FR4, of the ABTx272 antibody are located on either side of each of the CDRs in VH and VL region sequences shown supra. In particular, the four FRs of the ABTx272 antibody VH region are as follows:

```
  (SEQ ID NO: 426)
VH FR1: QVQLVQSGAAVKKPGESLKISCKGS;
  (SEQ ID NO: 427)
VH FR2: IGWVRQMPGKGLEWMGI;
and
  (SEQ ID NO: 428)
VH FR3: RYSPSFQGQVTISAGKSISTAYLQWSSLKASDTAMYYC
or
  (SEQ ID NO: 973)
YSPSFQGQVTISAGKSISTAYLQWSSLKASDTAMYYC;
(SEQ ID NO: 429)
VH FR4: WGQGTMVTVSS.
```

The four FRs of the ABTx272 antibody VL region are as follows:

```
  (SEQ ID NO: 430)
VL FR1: AIQLTQSPSSLSASVGDRVTITCRAS;
  (SEQ ID NO: 431)
VL FR2: LAWYQQKPGKAPKLLIY;
  (SEQ ID NO: 432)
VL FR3: SLESGVPSRFSGSGSGTDFTLTISSLQPEDFATYYC;
and
(SEQ ID NO: 433)
VL FR4: FGGGTKVEIKRTV.
```

By “ABTx272 polynucleotide” is meant a nucleic acid molecule (e.g., DNA) encoding at least a fragment of an ABTx272 antibody. In an embodiment, the encoded fragment has antigen binding activity.

By “ABTx273” is meant an antibody having at least about 85% amino acid sequence identity to an antibody sequence of antibody ABTx273 or comprising VH and/or VL CDRs 1-3 of ABTx273 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx273. Exemplary heavy chain and light chain sequences for antibody ABTx273 are provided below, where the variable regions are in plain text, the constant domains are in bold, and complementarity determining regions (CDRs), i.e., CDR1, CDR2, and CDR2, are underlined:

ABTx273 heavy chain (HC):
(SEQ ID NO: 1013)
QVQLVQSGAAVKKPGESLKISCKGSGYRFTSYWIGWVRQMPGKGLEWMGIIYPGDSDTRYSPSF
QGQVTISAGKSISTAYLQWSSLKASDTAMYYCARHGRGYDGYEGAFDIWGQGTMVTVSSASTKG
PSVFPLAPSSKSTSGGTAALGCLVKDYFPEPVTVSWNSGALTSGVHTFPAVLOSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSLS
LSPG
ABTx273 light chain (LC):
(SEQ ID NO: 1045)
AIQLTQSPSSLSASVGDRVTITCRASQGISSALAWYQQKPGKAPKLLIYDASSLESGVPSRFSG
SGSGTDFTLTISSLQPEDFATYYCQQFRSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC.

The three CDRs of the ABTx273 antibody VH region are as follows:

```
  (SEQ ID NO: 421)
VH CDR1: GYRFTSYW;
  (SEQ ID NO: 958)
VH CDR2: IYPGDSDTR;
and
  (SEQ ID NO: 948)
VH CDR3: ARHGRGYDGYEGAFDI.
```

The three CDRs of the ABTx0273 antibody VL region are as follows:

(SEQ ID NO: 424)
VL CDR1: QGISSA;
VL CDR2: DAS;
and
(SEQ ID NO: 953)
VL CDR3: QQFRSYPLT

The four framework (FR) regions, i.e., FR1, FR2, FR3, and FR4, of the ABTx273 antibody are located on either side of each of the CDRs in VH and VL region sequences shown supra. In particular, the four FRs of the ABTx273 antibody VH region are as follows:

```
  (SEQ ID NO: 426)
VH FRI: QVQLVQSGAAVKKPGESLKISCKGS;
  (SEQ ID NO: 427)
VH FR2: IGWVRQMPGKGLEWMGI;
and
  (SEQ ID NO: 428)
VH FR3: RYSPSFQGQVTISAGKSISTAYLOWSSLKASDTAMYYC
or
  (SEQ ID NO: 973)
YSPSFQGQVTISAGKSISTAYLQWSSLKASDTAMYYC;
(SEQ ID NO: 429)
VH FR4: WGQGTMVTVSS
```

The four FRs of the ABTx273 antibody VL region are as follows:

```
  (SEQ ID NO: 430)
VL FRI: AIQLTQSPSSLSASVGDRVTITCRAS;
  (SEQ ID NO: 431)
VL FR2: LAWYQQKPGKAPKLLIY;
  (SEQ ID NO: 432)
VL FR3: SLESGVPSRFSGSGSGTDFTLTISSLQPEDFATYYC;
and
(SEQ ID NO: 433)
VL FR4: FGGGTKVEIKRTV
```

By “ABTx273 polynucleotide” is meant a nucleic acid molecule (e.g., DNA) encoding at least a fragment of an ABTx273 antibody. In an embodiment, the encoded fragment has antigen binding activity.

By “ABTx274” is meant an antibody having at least about 85% amino acid sequence identity to an antibody sequence of antibody ABTx274 or comprising VH and/or VL CDRs 1-3 of ABTx274 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx274. Exemplary heavy chain and light chain sequences for antibody ABTx274 are provided below, where the variable regions are in plain text, the constant domains are in bold, and complementarity determining regions (CDRs), i.e., CDR1, CDR2, and CDR2, are underlined:

```
ABTx274 hevy chain (HC):
  (SEQ ID NO: 1016)
QVQLVQSGAAVKKPGESLKISCKGSGYRFTSYWIGWVRQMPGKGLEWMGIIYPGDSDTKYSPSF
QGQVTISAGKSISTAYLQWSSLKASDTAMYYCARHGRGYDGYEGAFDIWGQGTMVTVSSASTKG
PSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTOKSLS
LSPG
ABTx274 light chain (LC):
  (SEQ ID NO: 1031)
AIQLTQSPSSLSASVGDRVTITCRASQGISSALAWYQQKPGKAPKLLIYDASSLESGVPSRFSG
SGSGTDFTLTISSLQPEDFATYYCQQTNSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSENRGEC.
```

The three CDRs of the ABTx274 antibody VH region are as follows:

```
  (SEQ ID NO: 421)
VH CDR1: GYRFTSYW;
(SEQ ID NO: 972)
VH CDR2: IYPGDSDTK;
and
  (SEQ ID NO: 948)
VH CDR3: ARHGRGYDGYEGAFDI.
The three CDRs of the ABTx274 antibody
VL region are as follows:
  (SEQ ID NO: 424)
VL CDR1: QGISSA;
VL CDR2: DAS;
and
(SEQ ID NO: 1044)
VL CDR3: QQTNSYPLT
```

The four framework (FR) regions, i.e., FR1, FR2, FR3, and FR4, of the ABTx274 antibody are located on either side of each of the CDRs in VH and VL region sequences shown supra. In particular, the four FRs of the ABTx274 antibody VH region are as follows:

```
  (SEQ ID NO: 426)
VH FRI: QVQLVQSGAAVKKPGESLKISCKGS;
  (SEQ ID NO: 427)
VH FR2: IGWVRQMPGKGLEWMGI;
and
  (SEQ ID NO: 428)
VH FR3: RYSPSFQGQVTISAGKSISTAYLQWSSLKASDTAMYYC
or
  (SEQ ID NO: 973)
YSPSFQGQVTISAGKSISTAYLQWSSLKASDTAMYYC;
(SEQ ID NO: 429)
VH FR4: WGQGTMVTVSS
```

The four FRs of the ABTx274 antibody VL region are as follows:

```
  (SEQ ID NO: 430)
VL FR1: AIQLTQSPSSLSASVGDRVTITCRAS;
(SEQ ID NO: 431)
VL FR2: LAWYQQKPGKAPKLLI;
  (SEQ ID NO: 432)
VL FR3: SLESGVPSRFSGSGSGTDFTLTISSLQPEDFATYYC;
and
(SEQ ID NO: 433)
VL FR4: FGGGTKVEIKRTV
```

By “ABTx274 polynucleotide” is meant a nucleic acid molecule (e.g., DNA) encoding at least a fragment of an ABTx274 antibody. In an embodiment, the encoded fragment has antigen binding activity.

By “ABTx313” is meant an antibody having at least about 85% amino acid sequence identity to an antibody sequence of antibody ABTx313 or comprising VH and/or VL CDRs 1-3 of ABTx313 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx313. Exemplary heavy chain and light chain sequences for antibody ABTx313 are provided below, where the variable regions are in plain text, the constant domains are in bold, and complementarity determining regions (CDRs), i.e., CDR1, CDR2, and CDR2, are underlined:

ABTx313 heavy chain (HC):
(SEQ ID NO: 1051)
QVQLVQSGAAVKKPGESLKISCKGSGYRFTSYWIGWVRQMPGKGLEWMGIIYPGDSDTRYSPSF
QGQVTISAGKSISTAYLQWSSLKASDTAMYYCARHGRGYDGYDGAFDIWGQGTMVTVSSASTKG
PSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDT
LMISRTPEVTCVVVAVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTOKSLS
LSPGK
ABTx313 light chain (LC):
(SEQ ID NO: 1019)
AIQLTQSPSSLSASVGDRVTITCRASQGISSALAWYQQKPGKAPKLLIYDASSLESGVPSRFSG
SGSGTDFTLTISSLQPEDFATYYCQQFSSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSENRGEC

The three CDRs of the ABTx313 VH region are as follows:

```
  (SEQ ID NO: 421)
VH CDR-1: GYRFTSYW
  (SEQ ID NO: 1053)
VH CDR-2: IYPGDSDTR
(SEQ ID NO: 1012)
VH CDR-3: ARHGRGYDGYDGAFDI
```

The three CDRs of the ABTx313 VL region are as follows:

```
  (SEQ ID NO: 424)
VL CDR-1: QGISSA
VL CDR-2: DAS
  (SEQ ID NO: 1020)
VL CDR-3: QQFSSYPLT
```

The four framework (FR) regions, i.e., FR1, FR2, FR3, and FR4, of the ABTx313 antibody are located on either side of each of the CDRs in VH and VL region sequences shown supra. In particular, the four FRs of the ABTx313 antibody VH region are as follows:

```
  (SEQ ID NO: 426)
VH FR1: QVQLVQSGAAVKKPGESLKISCKGS
  (SEQ ID NO: 427)
VH FR2: IGWVRQMPGKGLEWMGI
  (SEQ ID NO: 1059)
VH FR3: YSPSFQGQVTISAGKSISTAYLQWSSLKASDTAMYYC
(SEQ ID NO: 429)
VH FR4: WGQGTMVTVSS
```

The four FRs of the ABTx313 antibody VL region are as follows:

```
  (SEQ ID NO: 430)
VL FR1: AIQLTQSPSSLSASVGDRVTITCRAS
  (SEQ ID NO: 431)
VL FR2: LAWYQQKPGKAPKLLIY
  (SEQ ID NO: 432)
VL FR3: SLESGVPSRFSGSGSGTDFTLTISSLQPEDFATYYC
(SEQ ID NO: 433)
VL FR4: FGGGTKVEIKRTV
```

By “ABTx313 polynucleotide” is meant a nucleic acid molecule (e.g., DNA) encoding at least a fragment of an ABTx313 antibody. In an embodiment, the encoded fragment has antigen binding activity.

By “ABTx307” is meant an antibody having at least about 85% amino acid sequence identity to an antibody sequence of antibody ABTx307 or comprising VH and/or VL CDRs 1-3 of ABTx307 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx307. Exemplary heavy chain and light chain sequences for antibody ABTx307 are provided below, where the variable regions are in plain text, the constant domains are in bold, and complementarity determining regions (CDRs), i.e., CDR1, CDR2, and CDR2, are underlined:

```
ABTx307 heavy chain (HC):
  (SEQ ID NO: 1051)
QVQLVQSGAAVKKPGESLKISCKGSGYRFTSYWIGWVRQMPGKGLEWMGIIYPGDSDTRYSPSF
QGQVTISAGKSISTAYLOWSSLKASDTAMYYCARHGRGYDGYDGAFDIWGQGTMVTVSSASTKG
PSVFPLAPSSKSTSGGTAALGCLVKDYFPEPVTVSWNSGALTSGVHTFPAVLOSSGLYSLSSVV
TVPSSSLGTQTYICNVNHKPSNTKVDKKVEPKSCDKTHTCPPCPAPEAAGGPSVFLFPPKPKDT
LMISRTPEVTCVVVAVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTOKSLS
LSPGK
ABTx307 light chain (LC) (VL):
  (SEQ ID NO: 420)
AIQLTQSPSSLSASVGDRVTITCRASQGISSALAWYQQKPGKAPKLLIYDASSLESGVPSRFSG
SGSGTDFTLTISSLQPEDFATYYCQQFNSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSENRGEC
```

The three CDRs of the ABTx307 VH region are as follows:

```
  (SEQ ID NO: 421)
VH CDR-1: GYRFTSYW
  (SEQ ID NO: 1053)
VH CDR-2: IYPGDSDTR
(SEQ ID NO: 1012)
VH CDR-3: ARHGRGYDGYDGAFDI
```

The three CDRs of the ABTx307 VL region are as follows:

(SEQ ID NO: 424)
VL CDR-1: QGISSA
VL CDR-2: DAS
(SEQ ID NO: 425)
VL CDR-3: QQFNSYPLT

The four framework (FR) regions, i.e., FR1, FR2, FR3, and FR4, of the ABTx307 antibody are located on either side of each of the CDRs in VH and VL region sequences shown supra. In particular, the four FRs of the ABTx307 antibody VH region are as follows:

```
  (SEQ ID NO: 426)
VH FR1: QVQLVQSGAAVKKPGESLKISCKGS
  (SEQ ID NO: 427)
VH FR2: IGWVRQMPGKGLEWMGI
  (SEQ ID NO: 1059)
VH FR3: YSPSFQGQVTISAGKSISTAYLQWSSLKASDTAMYYC
(SEQ ID NO: 429)
VH FR4: WGQGTMVTVSS
```

The four FRs of the ABTx307 antibody VL region are as follows:

```
  (SEQ ID NO: 430)
VL FRI: AIQLTQSPSSLSASVGDRVTITCRAS
  (SEQ ID NO: 431)
VL FR2: LAWYQQKPGKAPKLLIY
  (SEQ ID NO: 432)
VL FR3: SLESGVPSRFSGSGSGTDFTLTISSLQPEDFATYYC
(SEQ ID NO: 433)
VL FR4: FGGGTKVEIKRTV
```

By “ABTx307 polynucleotide” is meant a nucleic acid molecule (e.g., DNA) encoding at least a fragment of an ABTx307 antibody. In an embodiment, the encoded fragment has antigen binding activity.

By “ABTx308” is meant an antibody having at least about 85% amino acid sequence identity to an antibody sequence of antibody ABTx308 or comprising VH and/or VL CDRs 1-3 of ABTx308 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx308. Exemplary heavy chain and light chain sequences for antibody ABTx308 are provided below, where the variable regions are in plain text, the constant domains are in bold, and complementarity determining regions (CDRs), i.e., CDR1, CDR2, and CDR2, are underlined:

ABTx308 heavy chain (HC):
(SEQ ID NO: 1066)
QVQLVQSGAAVKKPGESLKISCKGSGYRFTSYWIGWVRQMPGKGLEWMGIIYPGDSDTKYSPSF
QGQVTISAGKSISTAYLQWSSLKASDTAMYYCARHGRGYDAYDGAFDIWGQGTMVTVSSASTKG
PSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDT
LMISRTPEVTCVVVAVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSLS
LSPGK
ABTx308 light chain (LC):
(SEQ ID NO: 1019)
AIQLTQSPSSLSASVGDRVTITCRASQGISSALAWYQQKPGKAPKLLIYDASSLESGVPSRFSG
SGSGTDFTLTISSLQPEDFATYYCQQFSSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC

The three CDRs of the ABTx308 VH region are as follows:

```
  (SEQ ID NO: 421)
VH CDR-1: GYRFTSYW
  (SEQ ID NO: 1063)
VH CDR-2: IYPGDSDTK
(SEQ ID NO: 1032)
VH CDR-3: ARHGRGYDAYDGAFDI
```

The three CDRs of the ABTx308 VL region are as follows:

(SEQ ID NO: 424)
VL CDR-1: QGISSA
VL CDR-2: DAS
(SEQ ID NO: 1020)
VL CDR-3: QQFSSYPLT

The four framework (FR) regions, i.e., FR1, FR2, FR3, and FR4, of the ABTx308 antibody are located on either side of each of the CDRs in VH and VL region sequences shown supra. In particular, the four FRs of the ABTx308 antibody VH region are as follows:

(SEQ ID NO: 426)
VH FRI: QVQLVQSGAAVKKPGESLKISCKGS
(SEQ ID NO: 427)
VH FR2: IGWVRQMPGKGLEWMGI
(SEQ ID NO: 1059)
VH FR3: YSPSFQGQVTISAGKSISTAYLQWSSLKASDTAMYYC
(SEQ ID NO: 429)
VH FR4: WGQGTMVTVSS

The four FRs of the ABTx308 antibody VL region are as follows:

```
  (SEQ ID NO: 1060)
VL FR1: AIQLTQSPSSLSASVGDRVTITCRAS
  (SEQ ID NO: 1061)
VL FR2: LAWYQQKPGKAPKLLIY
  (SEQ ID NO: 1062)
VL FR3: SLESGVPSRFSGSGSGTDFTLTISSLQPEDFATYYC
(SEQ ID NO: 433)
VL FR4: FGGGTKVEIKRTV
```

By “ABTx308 polynucleotide” is meant a nucleic acid molecule (e.g., DNA) encoding at least a fragment of an ABTx308 antibody. In an embodiment, the encoded fragment has antigen binding activity.

By “ABTx309” is meant an antibody having at least about 85% amino acid sequence identity to an antibody sequence of antibody ABTx309 or comprising VH and/or VL CDRs 1-3 of ABTx309 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx309. Exemplary heavy chain and light chain sequences for antibody ABTx309 are provided below, where the variable regions are in plain text, the constant domains are in bold, and complementarity determining regions (CDRs), i.e., CDR1, CDR2, and CDR2, are underlined:

ABTx309 heavy chain (HC):
(SEQ ID NO: 1067)
QVQLVQSGAAVKKPGESLKISCKGSGYRFTSYWIGWVRQMPGKGLEWMGIIYPGDSDTRYSPSF
QGQVTISAGKSISTAYLQWSSLKASDTAMYYCARHGRGYDAYDGAFDIWGQGTMVTVSSASTKG
PSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDT
LMISRTPEVTCVVVAVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSLS
LSPGK
ABTx309 light chain (LC):
(SEQ ID NO: 1031)
AIQLTQSPSSLSASVGDRVTITCRASQGISSALAWYQQKPGKAPKLLIYDASSLESGVPSRFSG
SGSGTDFTLTISSLQPEDFATYYCQQTNSYPLTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC

The three CDRs of the ABTx309 VH region are as follows:

VH CDR-1:
(SEQ ID NO: 421)
GYRFTSYW
VH CDR-2:
(SEQ ID NO: 1053)
IYPGDSDTR
VH CDR-3:
(SEQ ID NO: 1064)
ARHGRGYDAYDGAFDI

The three CDRs of the ABTx309 VL region are as follows:

VL CDR-1:
(SEQ ID NO: 424)
QGISSA
VL CDR-2:
DAS
VL CDR-3:
(SEQ ID NO: 1044)
QQTNSYPLT

The four framework (FR) regions, i.e., FR1, FR2, FR3, and FR4, of the ABTx309 antibody are located on either side of each of the CDRs in VH and VL region sequences shown supra. In particular, the four FRs of the ABTx309 antibody VH region are as follows:

VH FR1:
(SEQ ID NO: 1057)
QVQLVQSGAAVKKPGESLKISCKGS
VH FR2:
(SEQ ID NO: 1058)
IGWVRQMPGKGLEWMGI
VH FR3:
(SEQ ID NO: 1059)
YSPSFQGQVTISAGKSISTAYLQWSSLKASDTAMYYC
VH FR4:
(SEQ ID NO: 429)
WGQGTMVTVSS

The four FRs of the ABTx309 antibody VL region are as follows:

VL FR1:
(SEQ ID NO: 430)
AIQLTQSPSSLSASVGDRVTITCRAS
VL FR2:
(SEQ ID NO: 431)
LAWYQQKPGKAPKLLIY
VL FR3:
(SEQ ID NO: 432)
SLESGVPSRFSGSGSGTDFTLTISSLQPEDFATYYC
VL FR4:
(SEQ ID NO: 433)
FGGGTKVEIKRTV

By “ABTx309 polynucleotide” is meant a nucleic acid molecule (e.g., DNA) encoding at least a fragment of an ABTx309 antibody. In an embodiment, the encoded fragment has antigen binding activity.

By “ABTx196” is meant an antibody having at least about 85% amino acid sequence identity to an antibody sequence of antibody ABTx196 or comprising VH and/or VL CDRs 1-3 of ABTx196 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx196. Exemplary heavy chain variable region and light chain variable region sequences for antibody ABTx196 are provided below, where complementarity determining regions (CDRs), i.e., CDR1, CDR2, and CDR2, are underlined:

ABTx196 heavy chain variable region (VH):
(SEQ ID NO: 1120)
EVQLVESGGGLVQPGRSLRLSCAASGFTFDDYAMHWVRQAPGKGLEWVS
GISWNSGTIGYADSVKGRFTNSRDNAKNSLYLEMNSLRAEDTALYYCAK
DSPPGYCSSASCYGAFDIWGQGTMVTVSS
ABTx196 light chain variable region (VL):
(SEQ ID NO: 1068)
DVVMTQSPGTLSLSPGERATLSCRASQSVSSSYLAWYQQKPGQAPRLLI
YGASSRATGIPDRFSGSGSGTDFTLTISRLEPEDFAVYYCQQSETCLTF
GGGTKLEIK

The three CDRs of the ABTx196 VH region are as follows:

VH CDR-1:
(SEQ ID NO: 391)
GFTFDDYA
VH CDR-2:
(SEQ ID NO: 1070)
ISWNSGTIG
VH CDR-3:
(SEQ ID NO: 393)
AKDSPPGYCSSASCYGAFDI
The three CDRs of the ABTx196 VL region are as
follows:
VL CDR-1:
(SEQ ID NO: 394)
QSVSSSY
VL CDR-2:
GAS
VL CDR-3:
(SEQ ID NO: 1074)
QQSETCLT

The four framework (FR) regions, i.e., FR1, FR2, FR3, and FR4, of the ABTx196 antibody are located on either side of each of the CDRs in VH and VL region sequences shown supra. In particular, the four FRs of the ABT x196 antibody VH region are as follows:

VH FR1:
(SEQ ID NO: 396)
EVQLVESGGGLVQPGRSLRLSCAAS
VH FR2:
(SEQ ID NO: 397)
MHWVRQAPGKGLEWVSG
VH FR3:
(SEQ ID NO: 1077)
YADSVKGRFTNSRDNAKNSLYLEMNSLRAEDTALYYC
VH FR4:
(SEQ ID NO: 429)
WGQGTMVTVSS

The four FRs of the ABT x196 antibody VL region are as follows:

VL FR1:
(SEQ ID NO: 400)
DVVMTQSPGTLSLSPGERATLSCRAS
VL FR2:
(SEQ ID NO: 446)
LAWYQQKPGQAPRLLIY
VL FR3:
(SEQ ID NO: 402)
SRATGIPDRFSGSGSGTDFTLTISRLEPEDFAVYYC
VL FR4:
(SEQ ID NO: 1082)
FGGGTKLEIK

By “ABT x196 polynucleotide” is meant a nucleic acid molecule (e.g., DNA) encoding at least a fragment of an ABT x196 antibody. In an embodiment, the encoded fragment has antigen binding activity.

By “ABTx202” is meant an antibody having at least about 85% amino acid sequence identity to an antibody sequence of antibody ABTx202 or comprising VH and/or VL CDRs 1-3 of ABTx202 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx202. Exemplary heavy chain variable region and light chain variable region sequences for antibody ABTx202 are provided below, where complementarity determining regions (CDRs), i.e., CDR1, CDR2, and

CDR2, are underlined:

ABTx202 Heavy chain variable region (VH):
(SEQ ID NO: 1085)
EVQLVESGGGLVQPGRSLRLSCAASGFTFDDYAMHWVRQAPGKGLEWVS
GISWNSGTIGYADSVKGRFTSSRDNAKNSLYLEMNSLRAEDTALYYCAK
DSPPGYCSSASCYGAFDIWGQGTMVTVSS
ABTx202 light chain variable region (VL):
(SEQ ID NO: 1086)
EIVMTQSPGTLSLSPGERATLSCRASQSVSSSYLAWYQQKPGQAPRLLI
YGSSTRATGIPARFSGSGSGTEFALTISSLQSEDFAVYYCQQYNFWPYT
FGQGTKVEIK

The three CDRs of the ABTx202 VH region are as follows:

VH CDR-1:
(SEQ ID NO: 391)
GFTFDDYA
VH CDR-2:
(SEQ ID NO: 1070)
ISWNSGTIG
VH CDR-3:
(SEQ ID NO: 393)
AKDSPPGYCSSASCYGAFDI

The three CDRs of the ABTx202 Light chain variable region (VL) region are as follows:

VL CDR-1:
(SEQ ID NO: 394)
QSVSSSY
VL CDR-2:
GSS
VL CDR-3:
(SEQ ID NO: 1084)
QQYNFWPYT

The four framework (FR) regions, i.e., FR1, FR2, FR3, and FR4, of the ABTx202 antibody are located on either side of each of the CDRs in VH and VL region sequences shown supra. In particular, the four FRs of the ABTx202 antibody VH region are as follows:

VH FR1:
(SEQ ID NO: 396)
EVQLVESGGGLVQPGRSLRLSCAAS
VH FR2:
(SEQ ID NO: 397)
MHWVRQAPGKGLEWVSG
VH FR3:
(SEQ ID NO: 1065)
YADSVKGRFTSSRDNAKNSLYLEMNSLRAEDTALYYC
VH FR4:
(SEQ ID NO: 429)
WGQGTMVTVSS

The four FRs of the ABTx202 antibody VL region are as follows:

VL FR1:
(SEQ ID NO: 1087)
EIVMTQSPGTLSLSPGERATLSCRAS
VL FR2:
(SEQ ID NO: 446)
LAWYQQKPGQAPRLLIY
VL FR3:
(SEQ ID NO: 1080)
TRATGIPARFSGSGSGTEFALTISSLQSEDFAVYYC
VL FR4:
(SEQ ID NO: 1088)
FGQGTKVEIK

By “ABTx202 polynucleotide” is meant a nucleic acid molecule (e.g., DNA) encoding at least a fragment of an ABTx202 antibody. In an embodiment, the encoded fragment has antigen binding activity.

By “ABTx198” is meant an antibody having at least about 85% amino acid sequence identity to an antibody sequence of antibody ABTx198 or comprising VH and/or VL CDRs 1-3 of ABTx198 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx198. Exemplary heavy chain variable region and light chain variable region sequences for antibody ABTx198 are provided below, where complementarity determining regions (CDRs), i.e., CDR1, CDR2, and CDR2, are underlined:

ABTx198 heavy chain variable region (VH):
(SEQ ID NO: 1089)
EVQLVESGGGLVQPGRSLRLSCAASGFTFDDYAMHWVRQAPGKGL
EWVSGISWNSGTIGYADSVKGRFTNSRDNAKNSLYLEMNSLRAED
TALYYCAKDSPPGYCASASCYGAFDIWGQGTMVTVSS
ABTx198 light chain variable region (VL):
(SEQ ID NO: 1103)
DVVMTQSPGTLSLSPGERATLSCRASQSVSSSYLAWYQQKPGQAP
RLLIYGASSRATGIPDRFSGSGSGTDFTLTISRLEPEDFAVYYCQ
QDSLGLTFGGGTKLEIK

The three CDRs of the ABTx198 VH region are as follows:

VH CDR-1:
(SEQ ID NO: 391)
GFTFDDYA
VH CDR-2:
(SEQ ID NO: 1070)
ISWNSGTIG
VH CDR-3:
(SEQ ID NO: 1090)
AKDSPPGYCASASCYGAFDI

The three CDRs of the ABTx198 Light chain variable region (VL) region are as follows:

VL CDR-1:
(SEQ ID NO: 394)
QSVSSSY
VL CDR-2:
GAS
VL CDR-3:
(SEQ ID NO: 1091)
QQDSLGLT

The four framework (FR) regions, i.e., FR1, FR2, FR3, and FR4, of the ABTx198 antibody are located on either side of each of the CDRs in VH and VL region sequences shown supra. In particular, the four FRs of the ABTx198 antibody VH region are as follows:

VH FR1:
(SEQ ID NO: 396)
EVQLVESGGGLVQPGRSLRLSCAAS
VH FR2:
(SEQ ID NO: 397)
MHWVRQAPGKGLEWVSG
VH FR3:
(SEQ ID NO: 1077)
YADSVKGRFTNSRDNAKNSLYLEMNSLRAEDTALYYC
VH FR4:
(SEQ ID NO: 429)
WGQGTMVTVSS

The four FRs of the ABTx198 antibody VL region are as follows:

VL FR1:
(SEQ ID NO: 400)
DVVMTQSPGTLSLSPGERATLSCRAS
VL FR2:
(SEQ ID NO: 446)
LAWYQQKPGQAPRLLIY
VL FR3:
(SEQ ID NO: 402)
SRATGIPDRFSGSGSGTDFTLTISRLEPEDFAVYYC
VL FR4:
(SEQ ID NO: 1082)
FGGGTKLEIK

By “ABTx198 polynucleotide” is meant a nucleic acid molecule (e.g., DNA) encoding at least a fragment of an ABTx198 antibody. In an embodiment, the encoded fragment has antigen binding activity.

By “ABTx203” is meant an antibody having at least about 85% amino acid sequence identity to an antibody sequence of antibody ABTx203 or comprising VH and/or VL CDRs 1-3 of ABTx203 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx203. Exemplary heavy chain variable region and light chain variable region sequences for antibody ABTx203 are provided below, where complementarity determining regions (CDRs), i.e., CDR1, CDR2, and CDR2, are underlined:

ABTx203 heavy chain variable region (VH):
(SEQ ID NO: 1092)
EVQLVESGGGLVQPGRSLRLSCAASGFTFDDYAMHWVRQAP
GKGLEWVSGISWNSGTIGYADSVKGRFTNSRDNAKNSLYLE
MNSLRAEDTALYYCAKDWPSGFCSSAYCYGAFDIWGQGTMV
TVSS
ABTx203 light chain variable region (VL):
(SEQ ID NO: 1093)
DIVMTQSPSFLSASVGDRVTITCRASQGISSYLAWYQQKP
GKAPKLLIYAASTLQNGVPSRFSGSGSGTDFTLTITSLQP
EDFATYYCQQSYSTPYTFGQGTKLEIK

The three CDRs of the ABTx203 VH region are as follows:

VH CDR-1:
(SEQ ID NO: 391)
GFTFDDYA
VH CDR-2:
(SEQ ID NO: 1070)
ISWNSGTIG
VH CDR-3:
(SEQ ID NO: 1094)
AKDWPSGFCSSAYCYGAFDI

The three CDRs of the ABTx203 VL region are as follows:

VL CDR-1:
(SEQ ID NO: 469)
QGISSY
VL CDR-2:
AAS
VL CDR-3:
(SEQ ID NO: 1097)
QQSYSTPYT

The four framework (FR) regions, i.e., FR1, FR2, FR3, and FR4, of the ABTx203 antibody are located on either side of each of the CDRs in VH and VL region sequences shown supra. In particular, the four FRs of the ABTx203 antibody VH region are as follows:

VH FR1:
(SEQ ID NO: 396)
EVQLVESGGGLVQPGRSLRLSCAAS
VH FR2:
(SEQ ID NO: 397)
MHWVRQAPGKGLEWVSG
VH FR3:
(SEQ ID NO: 1077)
YADSVKGRFTNSRDNAKNSLYLEMNSLRAEDTALYYC
VH FR4:
(SEQ ID NO: 429)
WGQGTMVTVSS

The four FRs of the ABTx203 antibody VL region are as follows:

VL FR1:
(SEQ ID NO: 475)
DIVMTQSPSFLSASVGDRVTITCRAS
VL FR2:
(SEQ ID NO: 431)
LAWYQQKPGKAPKLLIY
VL FR3:
(SEQ ID NO: 1099)
TLQNGVPSRFSGSGSGTDFTLTITSLOPEDFATYYC
VL FR4:
(SEQ ID NO: 1100)
FGQGTKLEIK

By “ABTx203 polynucleotide” is meant a nucleic acid molecule (e.g., DNA) encoding at least a fragment of an ABTx203 antibody. In an embodiment, the encoded fragment has antigen binding activity.

By “ABTx205” is meant an antibody having at least about 85% amino acid sequence identity to an antibody sequence of antibody ABTx205 or comprising VH and/or VL CDRs 1-3 of ABTx205 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx205. Exemplary heavy chain variable region and light chain variable region sequences for antibody ABTx205 are provided below, where complementarity determining regions (CDRs), i.e., CDR1, CDR2, and CDR2, are underlined:

ABTx205 heavy chain variable region (VH):
(SEQ ID NO: 1120)
EVQLVESGGGLVQPGRSLRLSCAASGFTFDDYAMHWVRQAPGKGL
EWVSGISWNSGTIGYADSVKGRFTNSRDNAKNSLYLEMNSLRAED
TALYYCAKDSPPGYCSSASCYGAFDIWGQGTMVTVSS
ABTx205 light chain variable region (VL):
(SEQ ID NO: 1093)
DIVMTQSPSFLSASVGDRVTITCRASQGISSYLAWYQQKPGKAPK
LLIYAASTLQNGVPSRFSGSGSGTDFTLTITSLQPEDFATYYCQQ
SYSTPYTFGQGTKLEIK

The three CDRs of the ABTx205 VH region are as follows:

VH CDR-1:
(SEQ ID NO: 391)
GFTFDDYA
VH CDR-2:
(SEQ ID NO: 1070)
ISWNSGTIG
VH CDR-3:
(SEQ ID NO: 393)
AKDSPPGYCSSASCYGAFDI

The three CDRs of the ABTx205 VL region are as follows:

VL CDR-1:
(SEQ ID NO: 1095)
QGISSY
VL CDR-2:
AAS
VL CDR-3:
(SEQ ID NO: 1097)
QQSYSTPYT

The four framework (FR) regions, i.e., FR1, FR2, FR3, and FR4, of the ABTx205 antibody are located on either side of each of the CDRs in VH and VL region sequences shown supra. In particular, the four FRs of the ABTx205 antibody VH region are as follows:

VH FR1:
(SEQ ID NO: 396)
EVQLVESGGGLVQPGRSLRLSCAAS
VH FR2:
(SEQ ID NO: 397)
MHWVRQAPGKGLEWVSG
VH FR3:
(SEQ ID NO: 1077)
YADSVKGRFTNSRDNAKNSLYLEMNSLRAEDTALYYC
VH FR4:
(SEQ ID NO: 429)
WGQGTMVTVSS

The four FRs of the ABTx205 antibody VL region are as follows:

VL FR1:
(SEQ ID NO: 475)
DIVMTQSPSFLSASVGDRVTITCRAS
VL FR2:
(SEQ ID NO: 431)
LAWYQQKPGKAPKLLIY
VL FR3:
(SEQ ID NO: 1099)
TLQNGVPSRFSGSGSGTDFTLTITSLQPEDFATYYC
VL FR4:
(SEQ ID NO: 1100)
FGQGTKLEIK

By “ABTx205 polynucleotide” is meant a nucleic acid molecule (e.g., DNA) encoding at least a fragment of an ABTx205 antibody. In an embodiment, the encoded fragment has antigen binding activity.

By “ABTx206” is meant an antibody having at least about 85% amino acid sequence identity to an antibody sequence of antibody ABTx206 or comprising VH and/or VL CDRs 1-3 of ABTx206 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx206. Exemplary heavy chain variable region and light chain variable region sequences for antibody ABTx206 are provided below, where the variable regions are in plain text, the constant domains are in bold, and complementarity determining regions (CDRs), i.e., CDR1, CDR2, and CDR2, are underlined:

ABTx206 heavy chain variable region (VH):
(SEQ ID NO: 1101)
EVQLVESGGGLVQPGRSLRLSCAASGFTFDDYAMHWVRQA
PGKGLEWVSGISWNSGTIGYADSVKGRFTNSRDNAKNSLY
LEMNSLRAEDTALYYCAKDSPPGYCSSASCYGAFDIRGQG
TMVTVSS
ABTx206 light chain variable region (VL):
(SEQ ID NO: 1093)
DIVMTQSPSFLSASVGDRVTITCRASQGISSYLAWYQQK
PGKAPKLLIYAASTLQNGVPSRFSGSGSGTDFTLTITSL
QPEDFATYYCQQSYSTPYTFGQGTKLEIK

The three CDRs of the ABTx206 VH region are as follows:

VH CDR-1:
(SEQ ID NO: 391)
GFTFDDYA
VH CDR-2:
(SEQ ID NO: 1070)
ISWNSGTIG
VH CDR-3:
(SEQ ID NO: 393)
AKDSPPGYCSSASCYGAFDI

The three CDRs of the ABTx206 VL region are as follows:

VL CDR-1:
(SEQ ID NO: 1095)
QGISSY
VL CDR-2:
AAS
VL CDR-3:
(SEQ ID NO: 1097)
QQSYSTPYT

The four framework (FR) regions, i.e., FR1, FR2, FR3, and FR4, of the ABTx206 antibody are located on either side of each of the CDRs in VH and VL region sequences shown supra. In particular, the four FRs of the ABTx206 antibody VH region are as follows:

VH FR1:
(SEQ ID NO: 396)
EVQLVESGGGLVQPGRSLRLSCAAS
VH FR2:
(SEQ ID NO: 397)
MHWVRQAPGKGLEWVSG
VH FR3:
(SEQ ID NO: 1077)
YADSVKGRFTNSRDNAKNSLYLEMNSLRAEDTALYYC
VH FR4:
(SEQ ID NO: 1102)
RGQGTMVTVSS

The four FRs of the ABTx206 antibody VL region are as follows:

VL FR1:
(SEQ ID NO: 475)
DIVMTQSPSFLSASVGDRVTITCRAS
VL FR2:
(SEQ ID NO: 431)
LAWYQQKPGKAPKLLIY
VL FR3:
(SEQ ID NO: 1099)
TLQNGVPSRFSGSGSGTDFTLTITSLQPEDFATYYC
VL FR4:
(SEQ ID NO: 1100)
FGQGTKLEIK

By “ABTx206 polynucleotide” is meant a nucleic acid molecule (e.g., DNA) encoding at least a fragment of an ABTx206 antibody. In an embodiment, the encoded fragment has antigen binding activity.

By “adenine” or “9H-Purin-6-amine” is meant a purine nucleobase with the molecular formula C₅H₅N₅, having the structure

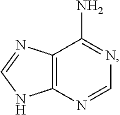

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2 (1H)-one” is meant an adenine molecule attached to a ribose sugar via a glycosidic bond, having the structure

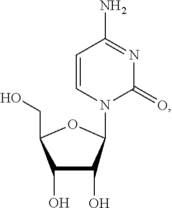

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE)” is meant a base editor comprising an adenosine deaminase.

By “Adenosine Base Editor (ABE) polynucleotide” is meant a polynucleotide encoding an ABE. By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by an oral route.

By “agent” is meant any small molecule chemical compound, antibody, nucleic acid molecule, or polypeptide, or fragments thereof.

“Allogeneic,” as used herein, refers to cells of the same species that differ genetically to the cell in comparison.

“Autologous,” as used herein, refers to cells from the same subject.

By “alteration” is meant a change in the level, structure, or activity of an analyte, gene or polypeptide as detected by standard art known methods such as those described herein. As used herein, an alteration includes a change (e.g., increase or decrease) in expression levels. In embodiments, the increase or decrease in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant decrease, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi-tri- and quad-specific antibodies, diabodies, triabodies, and tetrabodies), and antigen binding fragments of antibodies, including, for example, Fab′, F(ab′)2, Fab, Fv, rlgG, and scFv fragments. Unless otherwise indicated, the term “monoclonal antibody” (mAb) is meant to include both intact molecules, as well as antibody fragments (including, for example, Fab and F(ab′)2 fragments) that are capable of specifically binding to a target protein. As used herein, the Fab and F(ab′)2 fragments refer to antibody fragments that lack the Fc fragment of an intact antibody.

Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable regions of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)2 fragments, that are capable of specifically binding to a target protein. Antibodies may include chimeric antibodies; recombinant and engineered antibodies, and antigen binding fragments thereof. Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)2, a fragment of an antibody molecule containing a monovalent antigen-binding portion of an antibody molecule, obtained by treating an intact antibody with the enzyme pepsin (i.e., a dimer of Fab′ fragments held together by two disulfide bonds).

The term “antigen-binding fragment,” as used herein, refers to one or more portions or fragments of an antibody that retain the ability to specifically bind to a target antigen. In an embodiment, the target antigen is a CD117 variant polypeptide or peptide. The antigen-binding function of an antibody can be performed by fragments of a full-length antibody. The antibody fragments can be a Fab, F(ab′)2, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). Such antibody fragments can be obtained using conventional techniques known to those of skill in the art, and the fragments can be screened for utility in the same manner as intact antibodies.

Antigen-binding fragments can be produced by recombinant DNA techniques, enzymatic or chemical cleavage of intact immunoglobulins, or, in some cases, by chemical peptide synthesis procedures known in the art. In some embodiments, antigen-binding fragments (e.g., .g., Fab′, F(ab′)2, Fab, scFab, Fv, rlgG, and scFv fragments) of an anti-CD117 antibody, which are joined by a synthetic linker, are encompassed herein.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors include those sequences with about or at least about 85% sequence identity to any base editor sequence provided in the sequence listing, such as those corresponding to SEQ ID NOs: 2-11.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

By “base editing activity” is meant acting to chemically alter a base within a polynucleotide. In one embodiment, a first base is converted to a second base. In an embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain (e.g., an adenosine deaminase), and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is an adenine or adenosine base editor (ABE). In some embodiments, the base editor system (e.g., a base editor system comprising an adenosine deaminase) comprises a uracil glycosylase inhibitor or other agent or peptide that inhibits the inosine base excision repair system.

By “β-globin (HBB) polypeptide” is meant a polypeptide having at least about 85% amino acid sequence identity to NCBI Accession No. NP_000509, provided below, or a fragment thereof capable of forming a dimer with a HBA1 polypeptide. In particular embodiments, a β-globin protein comprises one or more alterations relative to the following reference sequence. In one particular embodiment, a β-globin protein associated with sickle cell disease comprises an E6V (also termed E7V) mutation.

(SEQ ID NO: 494)
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH.

By HBB polynucleotide” is meant a nucleic acid molecule that encodes an HBB polypeptide as well as the introns, exons, 3′ untranslated regions, 5′ untranslated regions, and regulatory sequences associated with its expression, or fragments thereof. In embodiments, a HBB polynucleotide is the genomic sequence, cDNA, mRNA, or gene associated with and/or required for HBB expression. Exemplary HBB polynucleotide sequences from Homo sapiens are provided below (NCBI Ref. Seq. Accessions No. NM_000518 and NG_059281).

(SEQ ID NO: 495)
ACATTTGCTTCTGACACAACTGTGTTCACTAGCAACCTCAAACAGACACC
ATGGTGCATCTGACTCCTGAGGAGAAGTCTGCCGTTACTGCCCTGTGGGG
CAAGGTGAACGTGGATGAAGTTGGTGGTGAGGCCCTGGGCAGGCTGCTGG
TGGTCTACCCTTGGACCCAGAGGTTCTTTGAGTCCTTTGGGGATCTGTCC
ACTCCTGATGCTGTTATGGGCAACCCTAAGGTGAAGGCTCATGGCAAGAA
AGTGCTCGGTGCCTTTAGTGATGGCCTGGCTCACCTGGACAACCTCAAGG
GCACCTTTGCCACACTGAGTGAGCTGCACTGTGACAAGCTGCACGTGGAT
CCTGAGAACTTCAGGCTCCTGGGCAACGTGCTGGTCTGTGTGCTGGCCCA
TCACTTTGGCAAAGAATTCACCCCACCAGTGCAGGCTGCCTATCAGAAAG
TGGTGGCTGGTGTGGCTAATGCCCTGGCCCACAAGTATCACTAAGCTCGC
TTTCTTGCTGTCCAATTTCTATTAAAGGTTCCTTTGTTCCCTAAGTCCAA
CTACTAAACTGGGGGATATTATGAAGGGCCTTGAGCATCTGGATTCTGCC
TAATAAAAAACATTTATTTTCATTGCAA.

>NG_059281.1:5001-6608 Homo sapiens hemoglobin subunit beta (HBB), RefSeqGene (LRG_1232) on chromosome 11; an A altered to T in Sickle cell disease is indicated in bold; the bold-underlined T indicates a SNP that is a C in some sickle cell patients. The underlined ATG is the start codon.

(SEQ ID NO: 940)
ACATTTGCTTCTGACACAACTGTGTTCACTAGCAACCTCAAACAGACACC
ATGGTGCATCTGACTCCTGAGGAGAAGTCTGCCGTTACTGCCCTGTGGGG
CAAGGTGAACGTGGATGAAGTTGGTGGTGAGGCCCTGGGCAGGTTGGTAT
CAAGGTTACAAGACAGGTTTAAGGAGACCAATAGAAACTGGGCATGTGGA
GACAGAGAAGACTCTTGGGTTTCTGATAGGCACTGACTCTCTCTGCCTAT
TGGTCTATTTTCCCACCCTTAGGCTGCTGGTGGTCTACCCTTGGACCCAG
AGGTTCTTTGAGTCCTTTGGGGATCTGTCCACTCCTGATGCTGTTATGGG
CAACCCTAAGGTGAAGGCTCATGGCAAGAAAGTGCTCGGTGCCTTTAGTG
ATGGCCTGGCTCACCTGGACAACCTCAAGGGCACCTTTGCCACACTGAGT
GAGCTGCACTGTGACAAGCTGCACGTGGATCCTGAGAACTTCAGGGTGAG
TCTATGGGACGCTTGATGTTTTCTTTCCCCTTCTTTTCTATGGTTAAGTT
CATGTCATAGGAAGGGGATAAGTAACAGGGTACAGTTTAGAATGGGAAAC
AGACGAATGATTGCATCAGTGTGGAAGTCTCAGGATCGTTTTAGTTTCTT
TTATTTGCTGTTCATAACAATTGTTTTCTTTTGTTTAATTCTTGCTTTCT
TTTTTTTTCTTCTCCGCAATTTTTACTATTATACTTAATGCCTTAACATT
GTGTATAACAAAAGGAAATATCTCTGAGATACATTAAGTAACTTAAAAAA
AAACTTTACACAGTCTGCCTAGTACATTACTATTTGGAATATATGTGTGC
TTATTTGCATATTCATAATCTCCCTACTTTATTTTCTTTTATTTTTAATT
GATACATAATCATTATACATATTTATGGGTTAAAGTGTAATGTTTTAATA
TGTGTACACATATTGACCAAATCAGGGTAATTTTGCATTTGTAATTTTAA
AAAATGCTTTCTTCTTTTAATATACTTTTTTGTTTATCTTATTTCTAATA
CTTTCCCTAATCTCTTTCTTTCAGGGCAATAATGATACAATGTATCATGC
CTCTTTGCACCATTCTAAAGAATAACAGTGATAATTTCTGGGTTAAGGCA
ATAGCAATATCTCTGCATATAAATATTTCTGCATATAAATTGTAACTGAT
GTAAGAGGTTTCATATTGCTAATAGCAGCTACAATCCAGCTACCATTCTG
CTTTTATTTTATGGTTGGGATAAGGCTGGATTATTCTGAGTCCAAGCTAG
GCCCTTTTGCTAATCATGTTCATACCTCTTATCTTCCTCCCACAGCTCCT
GGGCAACGTGCTGGTCTGTGTGCTGGCCCATCACTTTGGCAAAGAATTCA
CCCCACCAGTGCAGGCTGCCTATCAGAAAGTGGTGGCTGGTGTGGCTAAT
GCCCTGGCCCACAAGTATCACTAAGCTCGCTTTCTTGCTGTCCAATTTCT
ATTAAAGGTTCCTTTGTTCCCTAAGTCCAACTACTAAACTGGGGGATATT
ATGAAGGGCCTTGAGCATCTGGATTCTGCCTAATAAAAAACATTTATTTT
CATTGCAA.

A “binding polypeptide” refers to a polypeptide, or an antigen binding portion or fragment thereof, that has specificity for and specifically binds to a CD117 polypeptide. In an embodiment, a binding polypeptide is an anti-CD117 antibody or immunoglobulin or an antigen binding portion or fragment thereof.

By “hemoglobin, gamma A (HBG1) polypeptide” is meant a polypeptide having at least about 85% amino acid sequence identity to Genbank Accession No. CAA23771.1, provided below, or a fragment thereof capable of forming a protein complex with alpha hemoglobin subunits.

(SEQ ID NO: 496)
MGHFTEEDKATITSLWGKVNVEDAGGETLGRLLVVYPWTQRFFDSFGNLS
SASAIMGNPKVKAHGKKVLTSLGDAIKHLDDLKGTFAQLSELHCDKLHVD
PENFKLLGNVLVTVLAIHFGKEFTPEVQASWQKMVTAVASALSSRYH.

By HBG1 polynucleotide” is meant a nucleic acid molecule that encodes an HBG1 polypeptide as well as the introns, exons, 3′ untranslated regions, 5′ untranslated regions, and regulatory sequences associated with its expression, or fragments thereof. In embodiments, a HBG1 polynucleotide is the genomic sequence, cDNA, mRNA, or gene associated with and/or required for HBG1 expression. An exemplary HBB polynucleotide sequence from Homo sapiens is provided on ENSEMBL at accession no. GRCh38:11:5248044:5259425:1, the reverse-complement of which is provided below (SEQ ID NO: 497). In the below sequence, exons encoding HBG1 are shown in bold and an exemplary HBG1 promoter region corresponds to a region 5′ of the first exon encoding HBG1 (e.g., the first 100, 200, 300, or 400 nucleotides 5′ of the first exon), or portions thereof.

(SEQ ID NO: 497)
CCTTCTCACTTGGGTAGGCTCTGTCAGAGGGAAAGTCTAGGCCTCAAGGC
TGAGACTTTTGTCCCATGAGGTGTTCCCTTGATGTAGCACAGTCCCCCTT
TTCCTAGGCGTGGGGCTTCCTGAGAGCCGAACTGTAGTGATTGTTATCTC
TCTTCTGGATCTAGCCACCCATCAGGTCTACCAGACTCCAGGCTGGTACT
GGGGTTTGTCTGCACAGAGTCTTGTGACGTGAACCATCTGTGGGTCTCTC
AGCCATAGATACAACCACCTGCTCCAATGGAGGTGGCAGAGGATGAAATG
GACTCTGTGAGGGTCCTTACTTTTGGTTGTTCAATGCACTATTTTTGTGC
TGGTTGGCCTCCTGCCAGGAGGTGGCACTTTCTAGAAAGCATCAGCAGAG
GCAGTCAGGTGGTGGTGGCTGGGGGGGCTGGGGCACCCTAGAACTCCCAA
GAATATATGCCCTTTGTCTTCAGCTACCAGGGTGAGTAAGGAAGGACCAT
CAGGTGGGGGCAGGACTAGTCGTGTCTGAGCTCAGAGTCTCCTTGGGCAG
GTCTTTCTGTGGCTACTGTGGGAGGATGGGGGTGTAGTTTCCAGGTCAAT
GGATTTATGTTCCTAGGACAATTATGGCTGCCTCTGCTGTGTCATGCAGG
TCATCAGGAAAGTGGGGGAAAGCAAGCAGTCACGTGACTTGCCCAGCTCC
CATGCAACTCAAAAGGTTGGTCTCACTTCCAGCGTGCACCCTCCCCCGCA
ACAGCACCGAATCTGTTTCCATGCAGTCAGTGAGCAAGGCTGAGAACTTG
CCCCAGGCTACCAGCTGCGAAACCAAGTAGGGCTGTCCTACTTCCCTGCC
AGTGGAGTCTGCACACCAAATTCATGTCCCCCCACCAACCCCCCCACTGC
CCAGCCCCTAGATCTGGCCAGGTGGAGATTTTCTTTTTCCTGTCATCTTT
TCCCAGTTCCTCTGGCAGCCCTCCCAAATGACCCCTGTGAGGCAAGGCAG
AAATGGCTTCCTAGGGGACCCAGAGAGCCCACAGGGCTTTTCCCGCTGCT
TCCTCTACCCCTGTATTTTGCTTGGCCCTCTAAATTGACTCAGCTCCAGG
TAAGGTCAGAATCTTCTCCTGTGGTCTAGATCTTCAGGTTCCCCAGTGAG
GATGTGTGTTTGGGGGTAGACGGTCCCCCTTTTCCACTTCCACAGTTTGG
GCACTCACAATATTTGGGGTGTTTCCCGGGTCCTGCAGGAGCAATCTGCT
TCTTTCAGAGGGTGTGTGCGTTCTCTCAGCTTTCTTGATTTATTTCTGCA
GGTGGTTCTGCAAAAAAAATTCCTGATGGGAGACTTCACATGCTGCTCTG
TGCATCCGAGTGGGAGCTGCAATGTACTTCTGCTGCCTCCCATCTGCCAT
CACCCTCTAATTTGTCGGTAATATGCATTTTTAATCAATCTTTTTTTCTC
TCTCTCTCTTTTTCTTCTCCCCCAAAACTATACTGCCCTTTGATATCAAG
GAATCAAGGACGTGATGTTGAGGGGTGGGCAGTGGATACACTCTTTACCC
CTTAGGGAGCTATATCTAGATTTAGATATTGCCAATTCAAGATAACTTAA
TTGAAAGCAAATTCATAATGAATACACACACACACACACACATCTGCATG
ACAAGATTTTTAATAGTTGAAAGAATAACTAATAATTGTCCACAGGCAAT
AAGGGCTTTTTAAGCAAAACAGTTGTGATAAACAGGTCATTCTTAGAATA
GTAATCCAGCCAATAGTACAGGTTGCTTAGAGATTATGTCATTACCAGAG
TTAAAATTCTATAATGGCTTCTCACTCCCTACCACTGAGGACAAGTTTAT
GTCCTTAGGTTTATGCTTCCCTGAAACAATACCACCTGCTATTCTCCACT
TTACATATCAACGGCACTGGTTCTTTATCTAACTCTCTGGCACAGCAGGA
GTTTGTTTTCTTCTGCTTCAGAGCTTTGAATTTACTATTTCAGCTTCTAA
ACTTTATTTGGCAATGCCTTCCCATGGCAGATTCCTTCTGTCATTTTGCC
TCTGTTCGAATACTTTCTCCTTAATTTCATTCTTAGTTAATAATATCTGA
AATTATTTTGTTGTTTAACTTAATTATTAATTTTATGTATGTTCTACCTA
GATTATAATCTTCAGAGGAAAGTTTTATTCTCTGACTTATTTAACTTAAA
TGCCCACTACTTTAAAAATTATGACATTTATTTAACAGATATTTGCTGAA
CAAATGTTTGAAAATACATGGGAAAGAATGCTTGAAAACACTTGAAATTG
CTTGTGTAAAGAAACAGTTTTATCAGTTAGGATTTAATCAATGTCAGAAG
CAATGATATAGGAAAAATCGAGGAATAAGACAGTTATGGATAAGGAGAAA
TCAACAAACTCTTAAAAGATATTGCCTCAAAAGCATAAGAGGAAATAAGG
GTTTATACATGACTTTTAGAACACTGCCTTGGTTTTTGGATAAATGGGGA
AGTTGTTTGAAAACAGGAGGGATCCTAGATATTCCTTAGTCTGAGGAGGA
GCAATTAAGATTCACTTGTTTAGAGGCTGGGAGTGGTGGCTCACGCCTGT
AATCCCAGAATTTTGGGAGGCCAAGGCAGGCAGATCACCTGAGGTCAAGA
GTTCAAGACCAACCTGGCCAACATGGTGAAATCCCATCTCTACAAAAATA
CAAAAATTAGACAGGCATGATGGCAAGTGCCTGTAATCCCAGCTACTTGG
GAGGCTGAGGAAGGAGAATTGCTTGAACCTGGAAGGCAGGAGTTGCAGTG
AGCCGAGATCATACCACTGCACTCCAGCCTGGGTGACAGAACAAGACTCT
GTCTCAAAAAAAAAAAAGAGAGATTCAAAAGATTCACTTGTTTAGGCCTT
AGCGGGCTTAGACACCAGTCTCTGACACATTCTTAAAGGTCAGGCTCTAC
AAATGGAACCCAACCAGACTCTCAGATATGGCCAAAGATCTATACACACC
CATCTCACAGATCCCCTATCTTAAAGAGACCCTAATTTGGGTTCACCTCA
GTCTCTATAATCTGTACCAGCATACCAATAAAAATCTTTCTCACCCATCC
TTAGATTGAGAGAAGTCACTTATTATTATGTGAGTAACTGGAAGATACTG
ATAAGTTGACAAATCTTTTTCTTTCCTTTCTTATTCAACTTTTATTTTAA
CTTCCAAAGAACAAGTGCAATATGTGCAGCTTTGTTGCGCAGGTCAACAT
GTATCTTTCTGGTCTTTTAGCCGCCTAACACTTTGAGCAGATATAAGCCT
TACACAGGATTATGAAGTCTGAAAGGATTCCACCAATATTATTATAATTC
CTATCAACCTGATAGGTTAGGGGAAGGTAGAGCTCTCCTCCAATAAGCCA
GATTTCCAGAGTTTCTGACGTCATAATCTACCAAGGTCATGGATCGAGTT
CAGAGAAAAAACAAAAGCAAAACCAAACCTACCAAAAAATAAAAATCCCA
AAGAAAAAATAAAGAAAAAAACAGCATGAATACTTCCTGCCATGTTAAGT
GGCCAATATGTCAGAAACAGCACTGAGTTACAGATAAAGATGTCTAAACT
ACAGTGACATCCCAGCTGTCACAGTGTGTGGACTATTAGTCAATAAAACA
GTCCCTGCCTCTTAAGAGTTGTTTTCCATGCAAATACATGTCTTATGTCT
TAGAATAAGATTCCCTAAGAAGTGAACCTAGCATTTATACAAGATAATTA
ATTCTAATCCATAGTATCTGGTAAAGAGCATTCTACCATCATCTTTACCG
AGCATAGAAGAGCTACACCAAAACCCTGGGTCATCAGCCAGCACATACAC
TTATCCAGTGATAAATACACATCATCGGGTGCCTACATACATACCTGAAT
ATAAAAAAAATACTTTTGCTGAGATGAAACAGGCGTGATTTATTTCAAAT
AGGTACGGATAAGTAGATATTGAAGTAAGGATTCAGTCTTATATTATATT
ACATAACATTAATCTATTCCTGCACTGAAACTGTTGCTTTATAGGATTTT
TCACTACACTAATGAGAACTTAAGAGATAATGGCCTAAAACCACAGAGAG
TATATTCAAAGATAAGTATAGCACTTCTTATTTGGAAACCAATGCTTACT
AAATGAGACTAAGACGTGTCCCATCAAAAATCCTGGACCTATGCCTAAAA
CACATTTCACAATCCCTGAACTTTTCAAAAATTGGTACATGCTTTAACTT
TAAACTACAGGCCTCACTGGAGCTACAGACAAGAAGGTGAAAAACGGCTG
ACAAAAGAAGTCCTGGTATCTTCTATGGTGGGAGAAGAAAACTAGCTAAA
GGGAAGAATAAATTAGAGAAAAATTGGAATGACTGAATCGGAACAAGGCA
AAGGCTATAAAAAAAATTAAGCAGCAGTATCCTCTTGGGGGCCCCTTCCC
CACACTATCTCAATGCAAATATCTGTCTGAAACGGTCCCTGGCTAAACTC
CACCCATGGGTTGGCCAGCCTTGCCTTGACCAATAGCCTTGACAAGGCAA
ACTTGACCAATAGTCTTAGAGTATCCAGTGAGGCCAGGGGCCGGCGGCTG
GCTAGGGATGAAGAATAAAAGGAAGCACCCTTCAGCAGTTCCACACACTC
GCTTCTGGAACGTCTGAGGTTATCAATAAGCTCCTAGTCCAGACGCCATG
GGTCATTTCACAGAGGAGGACAAGGCTACTATCACAAGCCTGTGGGGCAA
GGTGAATGTGGAAGATGCTGGAGGAGAAACCCTGGGAAGGTAGGCTCTGG
TGACCAGGACAAGGGAGGGAAGGAAGGACCCTGTGCCTGGCAAAAGTCCA
GGTCGCTTCTCAGGATTTGTGGCACCTTCTGACTGTCAAACTGTTCTTGT
CAATCTCACAGGCTCCTGGTTGTCTACCCATGGACCCAGAGGTTCTTTGA
CAGCTTTGGCAACCTGTCCTCTGCCTCTGCCATCATGGGCAACCCCAAAG
TCAAGGCACATGGCAAGAAGGTGCTGACTTCCTTGGGAGATGCCATAAAG
CACCTGGATGATCTCAAGGGCACCTTTGCCCAGCTGAGTGAACTGCACTG
TGACAAGCTGCATGTGGATCCTGAGAACTTCAAGGTGAGTCCAGGAGATG
TTTCAGCACTGTTGCCTTTAGTCTCGAGGCAACTTAGACAACTGAGTATT
GATCTGAGCACAGCAGGGTGTGAGCTGTTTGAAGATACTGGGGTTGGGAG
TGAAGAAACTGCAGAGGACTAACTGGGCTGAGACCCAGTGGCAATGTTTT
AGGGCCTAAGGAGTGCCTCTGAAAATCTAGATGGACAACTTTGACTTTGA
GAAAAGAGAGGTGGAAATGAGGAAAATGACTTTTCTTTATTAGATTTCGG
TAGAAAGAACTTTCACCTTTCCCCTATTTTTGTTATTCGTTTTAAAACAT
CTATCTGGAGGCAGGACAAGTATGGTCATTAAAAAGATGCAGGCAGAAGG
CATATATTGGCTCAGTCAAAGTGGGGAACTTTGGTGGCCAAACATACATT
GCTAAGGCTATTCCTATATCAGCTGGACACATATAAAATGCTGCTAATGC
TTCATTACAAACTTATATCCTTTAATTCCAGATGGGGGCAAAGTATGTCC
AGGGGTGAGGAACAATTGAAACATTTGGGCTGGAGTAGATTTTGAAAGTC
AGCTCTGTGTGTGTGTGTGTGTGTGTGCGCGCGTGTGTTTGTGTGTGTGT
GAGAGCGTGTGTTTCTTTTAACGTTTTCAGCCTACAGCATACAGGGTTCA
TGGTGGCAAGAAGATAACAAGATTTAAATTATGGCCAGTGACTAGTGCTG
CAAGAAGAACAACTACCTGCATTTAATGGGAAAGCAAAATCTCAGGCTTT
GAGGGAAGTTAACATAGGCTTGATTCTGGGTGGAAGCTTGGTGTGTAGTT
ATCTGGAGGCCAGGCTGGAGCTCTCAGCTCACTATGGGTTCATCTTTATT
GTCTCCTTTCATCTCAACAGCTCCTGGGAAATGTGCTGGTGACCGTTTTG
GCAATCCATTTCGGCAAAGAATTCACCCCTGAGGTGCAGGCTTCCTGGCA
GAAGATGGTGACTGGAGTGGCCAGTGCCCTGTCCTCCAGATACCACTGAG
CTCACTGCCCATGATGCAGAGCTTTCAAGGATAGGCTTTATTCTGCAAGC
AATCAAATAATAAATCTATTCTGCTAAGAGATCACACATGGTTGTCTTCA
GTTCTTTTTTTATGTCTTTTTAAATATATGAGCCACAAAGGGTTTTATGT
TGAGGGATGTGTTTATGTGTATTTATACATGGCTATGTGTGTTTGTGTCA
TGTGCACACTCCACACTTTTTTGTTTACGTTAGATGTGGGTTTTGATGAG
CAAATAAAAGAACTAGGCAATAAAGAAACTTGTACATGGGAGTTCTGCAA
GTGGGAGTAAAAGGTGCAGGAGAAATCTGGTTGGAAGAAAGACCTCTATA
GGACAGGACTCCTCAGAAACAGATGTTTTGGAAGAGATGGGGAAAGGTTC
AGTGAAGGGGGCTGAACCCCCTTCCCTGGATTGCAGCACAGCAGCGAGGA
AGGGGCTCAACGAAGAAAAAGTGTTCCAAGCTTTAGGAAGTCAAGGTTTA
GGCAGGGATAGCCATTCTATTTTATTAGGGGCAATACTATTTCCAACGGC
ATCTGGCTTTTCTCAGCCCTTGTGAGGCTCTACAGGGAGGTTGAGGTGTT
AGAGATCAGAGCAGGAAACAGGTTTTTCTTTCCACGGTAACTACAATGAA
GTGATCCTTACTTTACTAAGGAACTTTTCATTTTAAGTGTTGACGCATGC
CTAAAGAGGTGAAATTAATCCCATACCCTTAAGTCTACAGACTGGTCACA
GCATTTCAAGGAGGAGACCTCATTGTAAGCTTCTAGGGAGGTGGGGACTT
AGGTGAAGGAAATGAGCCAGCAGAAGCTCACAAGTCAGCATCAGCGTGTC
ATGTCTCAGCAGCAGAACAGCACGGTCAGATGAAAATATAGTGTGAAGAA
TTTGTATAACATTAATTGAGAAGGCAGATTCACTGGAGTTCTTATATAAT
TGAAAGTTAATGCACGTTAATAAGCAAGAGTTTAGTTTAATGTGATGGTG
TTATGAACTTAACGCTTGTGTCTCCAGAAAATTCACATGCTGAATCCCCA
ACTCCCAATTGGCTCCATTTGTGGGGGAGGCTTTGGAAAAGTAATCAGGT
TTAGAGGAGCTCATGAGAGCAGATCCCCATCATAGAATTATTTTCCTCAT
CAGAAGCAGAGAGATTAGCCATTTCTCTTCCTTCTGGTGAGGACACAGTG
GGAAGTCAGCCACCTGCAACCCAGGAAGAGAGCCCTGACCAGGAACCAGC
AGAAAAGTGAGAAAAAATCCTGTTGTTGAAGTCACCCAGTCTATGCTATT
TTGTTATAGCACCTTGCACTAAGTAAGGCAGATGAAGAAAGAGAAAAAAA
TAAGCTTCGGTGTTCAGTGGATTAGAAACCATGTTTATCTCAGGTTTACA
AATCTCCACTTGTCCTCTGTGTTTCAGAATAAAATACCAACTCTACTACT
CTCATCTGTAAGATGCAAATAGTAAGCCTGAGCCCTTCTGTCTAACTTTG
AATTCTATTTTTTCTTCAACGTACTTTAGGCTTGTAATGTGTTTATATAC
AGTGAAATGTCAAGTTCTTTCTTTATATTTCTTTCTTTCTTTTTTTTCCT
CAGCCTCAGAGTTTTCCACATGCCCTTCCTACTTTCAGGAACTTCTTTCT
CCAAACGTCTTCTGCCTGGCTCCATCAAATCATAAAGGACCCACTTCAAA
TGCCATCACTCACTACCATTTCACAATTCGCACTTTCTTTCTTTGTCCTT
TTTTTTTTTAGTAAAACAAGTTTATAAAAAATTGAAGGAATAAATGAATG
GCTACTTCATAGGCAGAGTAGACGCAAGGGCTACTGGTTGCCGATTTTTA
TTGTTATTTTTCAATAGTATGCTAAACAAGGGGTAGATTATTTATGCTGC
CCATTTTTAGACCATAAAAGATAACTTCCTGATGTTGCCATGGCATTTTT
TTCCTTTTAATTTTATTTCATTTCATTTTAATTTCGAAGGTACATGTGCA
GGATGTGCAGGCTTGTTACATGGGTAAATGTGTGTCTTTCTGGCCTTTTA
GCCATCTGTATCAATGAGCAGATATAAGCTTTACACAGGATCATGAAGGA
TGAAAGAATTTCACCAATATTATAATAATTTCAATCAACCTGATAGCTTA
GGGGATAAACTAATTTGAAGATACAGCTTGCCTCCGATAAGCCAGAATTC
CAGAGCTTCTGGCATTATAATCTAGCAAGGTTAGAGATCATGGATCACTT
TCAGAGAAAAACAAAAACAAACTAACCAAAAGCAAAACAGAACCAAAAAA
CCACCATAAATACTTCCTACCCTGTTAATGGTCCAATATGTCAGAAACAG
CACTGTGTTAGAAATAAAGCTGTCTAAAGTACACTAATATTCGAGTTATA
ATAGTGTGTGGACTATTAGTCAATAAAAACAACCCTTGCCTCTTTAGAGT
TGTTTTCCATGTACACGCACATCTTATGTCTTAGAGTAAGATTCCCTGAG
AAGTGAACCTAGCATTTATACAAGATAATTAATTCTAATCCACAGTACCT
GCCAAAGAACATTCTACCATCATCTTTACTGAGCATAGAAGAGCTACGCC
AAAACCCTGGGTCATCAGCCAGCACACACACTTATCCAGTGGTAAATACA
CATCATCTGGTGTATACATACATACCTGAATATGGAATCAAATATTTTTC
TAAGATGAAACAGTCATGATTTATTTCAAATAGGTACGGATAAGTAGATA
TTGAGGTAAGCATTAGGTCTTATATTATGTAACACTAATCTATTACTGCG
CTGAAACTGTGGCTTTATAGAAATTGTTTTCACTGCACTATTGAGAAATT
AAGAGATAATGGCAAAAGTCACAAAGAGTATATTCAAAAAGAAGTATAGC
ACTTTTTCCTTAGAAACCACTGCTAACTGAAAGAGACTAAGATTTGTCCC
GTCAAAAATCCTGGACCTATGCCTAAAACACATTTCACAATCCCTGAACT
TTTCAAAAATTGGTACATGCTTTAGCTTTAAACTACAGGCCTCACTGGAG
CTAGAGACAAGAAGGTAAAAAACGGCTGACAAAAGAAGTCCTGGTATCCT
CTATGATGGGAGAAGGAAACTAGCTAAAGGGAAGAATAAATTAGAGAAAA
ACTGGAATGACTGAATCGGAACAAGGCAAAGGCTATAAAAAAAATTAGCA
GTATCCTCTTGGGGGCCCCTTCCCCACACTATCTCAATGCAAATATCTGT
CTGAAACGGTCCCTGGCTAAACTCCACCCATGGGTTGGCCAGCCTTGCCT
TGACCAATAGCCTTGACAAGGCAAACTTGACCAATAGTCTTAGAGTATCC
AGTGAGGCCAGGGGCCGGCGGCTGGCTAGGGATGAAGAATAAAAGGAAGC
ACCCTTCAGCAGTTCCACACACTCGCTTCTGGAACGTCTGAGGTTATCAA
TAAGCTCCTAGTCCAGACGCCATGGGTCATTTCACAGAGGAGGACAAGGC
TACTATCACAAGCCTGTGGGGCAAGGTGAATGTGGAAGATGCTGGAGGAG
AAACCCTGGGAAGGTAGGCTCTGGTGACCAGGACAAGGGAGGGAAGGAAG
GACCCTGTGCCTGGCAAAAGTCCAGGTCGCTTCTCAGGATTTGTGGCACC
TTCTGACTGTCAAACTGTTCTTGTCAATCTCACAGGCTCCTGGTTGTCTA
CCCATGGACCCAGAGGTTCTTTGACAGCTTTGGCAACCTGTCCTCTGCCT
CTGCCATCATGGGCAACCCCAAAGTCAAGGCACATGGCAAGAAGGTGCTG
ACTTCCTTGGGAGATGCCACAAAGCACCTGGATGATCTCAAGGGCACCTT
TGCCCAGCTGAGTGAACTGCACTGTGACAAGCTGCATGTGGATCCTGAGA
ACTTCAAGGTGAGTCCAGGAGATGTTTCAGCCCTGTTGCCTTTAGTCTCG
AGGCAACTTAGACAACGGAGTATTGATCTGAGCACAGCAGGGTGTGAGCT
GTTTGAAGATACTGGGGTTGGGGGTGAAGAAACTGCAGAGGACTAACTGG
GCTGAGACCCAGTGGTAATGTTTTAGGGCCTAAGGAGTGCCTCTAAAAAT
CTAGATGGACAATTTTGACTTTGAGAAAAGAGAGGTGGAAATGAGGAAAA
TGACTTTTCTTTATTAGATTCCAGTAGAAAGAACTTTCATCTTTCCCTCA
TTTTTGTTGTTTTAAAACATCTATCTGGAGGCAGGACAAGTATGGTCGTT
AAAAAGATGCAGGCAGAAGGCATATATTGGCTCAGTCAAAGTGGGGAACT
TTGGTGGCCAAACATACATTGCTAAGGCTATTCCTATATCAGCTGGACAC
ATATAAAATGCTGCTAATGCTTCATTACAAACTTATATCCTTTAATTCCA
GATGGGGGCAAAGTATGTCCAGGGGTGAGGAACAATTGAAACATTTGGGC
TGGAGTAGATTTTGAAAGTCAGCTCTGTGTGTGTGTGTGTGTGTGCGCGC
GCGCGTGTGTGTGTGTGTGTCAGCGTGTGTTTCTTTTAACGTCTTCAGCC
TACAACATACAGGGTTCATGGTGGCAAGAAGATAGCAAGATTTAAATTAT
GGCCAGTGACTAGTGCTTGAAGGGGAACAACTACCTGCATTTAATGGGAA
GGCAAAATCTCAGGCTTTGAGGGAAGTTAACATAGGCTTGATTCTGGGTG
GAAGCTTGGTGTGTAGTTATCTGGAGGCCAGGCTGGAGCTCTCAGCTCAC
TATGGGTTCATCTTTATTGTCTCCTTTCATCTCAACAGCTCCTGGGAAAT
GTGCTGGTGACCGTTTTGGCAATCCATTTCGGCAAAGAATTCACCCCTGA
GGTGCAGGCTTCCTGGCAGAAGATGGTGACTGCAGTGGCCAGTGCCCTGT
CCTCCAGATACCACTGAGCTCACTGCCCATGATTCAGAGCTTTCAAGGAT
AGGCTTTATTCTGCAAGCAATACAAATAATAAATCTATTCTGCTGAGAGA
TCACACATGATTTTCTTCAGCTCTTTTTTTTACATCTTTTTAAATATATG
AGCCACAAAGGGTTTATATTGAGGGAAGTGTGTATGTGTATTTCTGCATG
CCTGTTTGTGTTTGTGGTGTGTGCATGCTCCTCATTTATTTTTATATGAG
ATGTGCATTTTGATGAGCAAATAAAAGCAGTAAAGACACTTGTACACGGG
AGTTCTGCAAGTGGGAGTAAATGGTGTAGGAG.

By “hemoglobin, gamma G (HBG2) polypeptide” is meant a polypeptide having at least about 85% amino acid sequence identity to Genbank Accession No. CAA23773.1, provided below, or a fragment thereof capable of forming a protein complex with alpha hemoglobin subunits.

(SEQ ID NO: 498)
MGHFTEEDKATITSLWGKVNVEDAGGETLGRLLVVYPWTQRFFDSFGNLS
SASAIMGNPKVKAHGKKVLTSLGDAIKHLDDLKGTFAQLSELHCDKLHVD
PENFKLLGNVLVTVLAIHFGKEFTPEVQASWQKMVTGVASALSSRYH.

By HBG2 polynucleotide” is meant a nucleic acid molecule that encodes an HBG2 polypeptide as well as the introns, exons, 3′ untranslated regions, 5′ untranslated regions, and regulatory sequences associated with its expression, or fragments thereof. In embodiments, a HBG2 polynucleotide is the genomic sequence, cDNA, mRNA, or gene associated with and/or required for HBG2 expression. An exemplary HBB polynucleotide sequence from Homo sapiens is provided on ENSEMBL at accession no. GRCh38:11:5248044:5259425:1, the reverse-complement of which is provided above (SEQ ID NO: 497). In the above sequence (SEQ ID NO: 497), exons encoding HBG2 are shown in bold-underlined text and an exemplary HBG2 promoter region corresponds to a region 5′ of the first exon encoding HBG2 (e.g., the first 100, 200, 300, or 400 nucleotides 5′ of the first exon), or portions thereof.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “percent chimerism” or “chimerism” is meant the proportion of cells of a given type(s) of interest in a subject that were administered to the subject or altered in the subject or that are descended or derived from the cells administered to or edited in the subject. In some cases, percent chimerism is calculated as the percent of hCD45+ cells in a subject that were administered to the subject or derived or descended from the cells administered to the subject. In embodiments, the cell type of interest is bulk bone marrow, CD34+ cells, CD235a+ cells, CD19+ cells, or CD45+ cells. In embodiments, chimerism is measured in a subject 1 day, 1 wk, 2 wks, 3 wks, 4 wks, 5 wks, 6 wks, 7 wks, 8 wks, 9 wks, 10 wks, 11 wks, 12 wks, 6 months, a year, or longer following administration of cells to the subject. In embodiments, chimerism is measured in a subject 1 day, 1 wk, 2 wks, 3 wks, 4 wks, 5 wks, 6 wks, 7 wks, 8 wks, 9 wks, 10 wks, 11 wks, 12 wks, 6 months, a year, or longer following administration of an anti-CD117 antibody to the subject. In embodiments, the percent chimerism measured at the time point is about or at least about 10%, 20%, 30%, 40%, 50%, 60%, 70%, 80%, 90%, or 100%. In some cases, the percent chimerism is greater than that measured for a subject administered cells expressing a wild type CD117 polypeptide and not base edited according to the methods provided herein to express an altered CD117 polypeptide with reduced binding to an anti-CD117 antibody. As used herein, the term “complementarity determining region” (CDR) refers to a hypervariable region found both in the light chain and the heavy chain variable regions ((VL and VH domains, respectively). CDRs are noncontiguous antigen-binding sites found within the variable regions of both heavy and light chain polypeptides. These particular regions have been described by Kabat et al., J. Biol. Chem. 252:6609-6616, 1977 and Kabat, et al., Sequences of Proteins of Immunological Interest, Fifth Edition, U.S. Department of Health and Human Services, NIH Publication No. 91-3242, 1991; by Chothia et al., (J. Mol. Biol. 196:901-917, 1987), and by MacCallum et al., (J. Mol. Biol. 262:732-745, 1996) where the definitions include overlapping or subsets of amino acid residues when compared against each other. In certain embodiments, the term “CDR” is a CDR as defined by Kabat based on sequence comparisons. The more highly conserved portions of variable regions are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The variable regions of native heavy and light chains each comprise four framework regions (FR1, FR2, FR3, FR4) that primarily adopt a beta-sheet configuration, connected by three CDRs (CDR1, CDR2, CDR3), which form loops that connect, and in some cases form part of, the beta-sheet structure. The CDRs in each chain are held together in close proximity by the FR regions in the order FR1-CDR1-FR2-CDR2-FR3-CDR3-FR4, and the CDRs in each antibody chain contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated. In various embodiments, complementarity determining regions are identified using any of the methodologies available to one of skill in the art such as those methods described in “Antibody structure-Function Relationships.” Therapeutic Antibody Engineering, edited by William R. Strohl and Lilia M. Strohl, Woodhead Publishing Series in Biomedicine, 2012, 37-56, 459-595, the entirety of which is incorporated herein in its entirety for all purposes, where such methods include, as non-limiting examples, those of Kabat, Chothia, Lefranc, Honegger, Martin, MacCallum, and Zhao. CDRs can be identified using sequence or structure based methods. Various software programs are available to one of skill in the art to identify CDRs for an antibody amino acid sequence. In various embodiments, a CDR as provided herein may be modified to include 1, 2, 3, 4, 5, 6, 7, 8, 9, or 10 additional amino acids and/or to exclude 1, 2, 3, 4, 5, 6, 7, 8, or 9 amino acids at the N-terminal and/or C-terminal end (e.g, in an embodiment VH CDR1 of ABTx025 is modified to be RASQSVSS (SEQ ID NO: 939), rather than QSVSSSY (SEQ ID NO: 438) by extending QSVSSSY (SEQ ID NO: 439) by 3 amino acids at the N-terminus and excluding two amino acids at the C-terminus). The present disclosure contemplates that the CDRs identified for a particular antibody can vary in location or length depending upon the method by which they are determined.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free-OH can be maintained; and glutamine for asparagine such that a free-NH₂ can be maintained.

As used herein, the terms “condition” and “conditioning” refer to processes by which a patient is prepared for receipt of a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation). According to the methods described herein, a patient may be conditioned for hematopoietic stem cell transplant therapy by administration to the patient of an antibody or antigen-binding fragment thereof capable of binding an antigen expressed by hematopoietic stem cells, such as CD117. Such antibodies are expected to act via complement-mediated cytotoxicity and antibody-dependent cell-mediated cytotoxicity. As described herein, the transplanted cells have been edited so that the antibody no longer binds a CD117 antigen. Administration of an antibody, antigen-binding fragment thereof, drug-antibody conjugate, or chimeric antigen receptor expressing T-cell (CAR-T) capable of binding a CD117 antigen to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2 (1H)-one” is meant a purine nucleobase with the molecular formula C₄H₅N₃O, having the structure

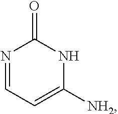

and corresponding to CAS No. 71-30-7.

By “cytidine” is meant a cytosine molecule attached to a ribose sugar via a glycosidic bond, having the structure

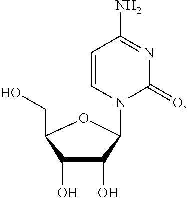

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide comprising a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include diseases amenable to treatment with hematopoietic stem cell transplantation, such as β-thalassemia, sickle cell disease (SCD), or adenosine deaminase deficiency.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity.

By “effective amount” is meant the amount of an agent or active compound, e.g., a base editor or antibody as described herein, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice embodiments of the disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

As used herein, the term “framework region” or “FR region” includes amino acid residues that are adjacent to the CDRs. FR region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter1 19, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche (e.g., the bone marrow niche) and re-establish productive and sustained hematopoiesis.”

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally occurring organic molecules with which it is naturally associated. In some embodiments, the preparation is at least 75%, 90%, or 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “cluster of differentiation 117 (CD117; C-kit; SCFR) polypeptide” is meant a polypeptide having at least about 85% amino acid sequence identity to an amino acid sequence provided at GenBank Accession No. NP_000213, which is provided below, or a fragment thereof that binds an anti-CD117 antibody. CD117 (KIT) is a type III receptor tyrosine kinase operating in cell signal transduction in several cell types. Normally KIT is activated (phosphorylated) by binding of its ligand, the stem cell factor (SCF). This leads to a phosphorylation cascade ultimately activating various transcription factors in different cell types. Such activation regulates apoptosis, cell differentiation, proliferation, chemotaxis, and cell adhesion. In some embodiments, an CD117 polypeptide or fragment thereof has SCF signaling activity.

>NP_000213.1 mast/stem cell growth factor
receptor Kit isoform 1 precursor [Homo sapiens]
(SEQ ID NO: 499)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHINGTVECKAYNDVGK
TSAYFNFAFKGNNKEQIHPHTLFTPLLIGFVIVAGMMCIIVMILTYKYLQ
KPMYEVQWKVVEEINGNNYVYIDPTQLPYDHKWEFPRNRLSFGKTLGAGA
FGKVVEATAYGLIKSDAAMTVAVKMLKPSAHLTEREALMSELKVLSYLGN
HMNIVNLLGACTIGGPTLVITEYCCYGDLLNFLRRKRDSFICSKQEDHAE
AALYKNLLHSKESSCSDSTNEYMDMKPGVSYVVPTKADKRRSVRIGSYIE
RDVTPAIMEDDELALDLEDLLSFSYQVAKGMAFLASKNCIHRDLAARNIL
LTHGRITKICDFGLARDIKNDSNYVVKGNARLPVKWMAPESIFNCVYTFE
SDVWSYGIFLWELFSLGSSPYPGMPVDSKFYKMIKEGFRMLSPEHAPAEM
YDIMKTCWDADPLKRPTFKQIVQLIEKQISESTNHIYSNLANCSPNRQKP
VVDHSVRINSVGSTASSSQPLLVHDDV
>CD117 variant with S261G alteration and
N260D alteration (shown in bold-underline)
(SEQ ID NO: 1134)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYDGWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV
>CD117 variant with S261G alteration
(shown in bold-underline)
(SEQ ID NO: 1135)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNGWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV
>CD117 variant with Y259C and N260D
alterations (shown in bold-underline)
(SEQ ID NO: 1136)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKCDSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHINGTVECKAYNDVGK
TSAYFNFAFKGNNKEQIHPHTLFTPLLIGFVIVAGMMCIIVMILTYKYLQ
KPMYEVQWKVVEEINGNNYVYIDPTQLPYDHKWEFPRNRLSFGKTLGAGA
FGKVVEATAYGLIKSDAAMTVAVKMLKPSAHLTEREALMSELKVLSYLGN
HMNIVNLLGACTIGGPTLVITEYCCYGDLLNFLRRKRDSFICSKQEDHAE
AALYKNLLHSKESSCSDSTNEYMDMKPGVSYVVPTKADKRRSVRIGSYIE
RDVTPAIMEDDELALDLEDLLSFSYQVAKGMAFLASKNCIHRDLAARNIL
LTHGRITKICDFGLARDIKNDSNYVVKGNARLPVKWMAPESIFNCVYTFE
SDVWSYGIFLWELFSLGSSPYPGMPVDSKFYKMIKEGFRMLSPEHAPAEM
YDIMKTCWDADPLKRPTFKQIVQLIEKQISESTNHIYSNLANCSPNRQKP
VVDHSVRINSVGSTASSSQPLLVHDDV
>CD117 variant with an N260D alteration
(shown in bold-underline)
(SEQ ID NO: 1137)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYDSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHINGTVECKAYNDVGK
TSAYFNFAFKGNNKEQIHPHTLFTPLLIGFVIVAGMMCIIVMILTYKYLQ
KPMYEVQWKVVEEINGNNYVYIDPTQLPYDHKWEFPRNRLSFGKTLGAGA
FGKVVEATAYGLIKSDAAMTVAVKMLKPSAHLTEREALMSELKVLSYLGN
HMNIVNLLGACTIGGPTLVITEYCCYGDLLNFLRRKRDSFICSKQEDHAE
AALYKNLLHSKESSCSDSTNEYMDMKPGVSYVVPTKADKRRSVRIGSYIE
RDVTPAIMEDDELALDLEDLLSFSYQVAKGMAFLASKNCIHRDLAARNIL
LTHGRITKICDFGLARDIKNDSNYVVKGNARLPVKWMAPESIFNCVYTFE
SDVWSYGIFLWELFSLGSSPYPGMPVDSKFYKMIKEGFRMLSPEHAPAEM
YDIMKTCWDADPLKRPTFKQIVQLIEKQISESTNHIYSNLANCSPNRQKP
VVDHSVRINSVGSTASSSQPLLVHDDV
>CD117 variant with an S251G alteration
(shown in bold-underline)
(SEQ ID NO: 1138)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
GQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHINGTVECKAYNDVGK
TSAYFNFAFKGNNKEQIHPHTLFTPLLIGFVIVAGMMCIIVMILTYKYLQ
KPMYEVQWKVVEEINGNNYVYIDPTQLPYDHKWEFPRNRLSFGKTLGAGA
FGKVVEATAYGLIKSDAAMTVAVKMLKPSAHLTEREALMSELKVLSYLGN
HMNIVNLLGACTIGGPTLVITEYCCYGDLLNFLRRKRDSFICSKQEDHAE
AALYKNLLHSKESSCSDSTNEYMDMKPGVSYVVPTKADKRRSVRIGSYIE
RDVTPAIMEDDELALDLEDLLSFSYQVAKGMAFLASKNCIHRDLAARNIL
LTHGRITKICDFGLARDIKNDSNYVVKGNARLPVKWMAPESIFNCVYTFE
SDVWSYGIFLWELFSLGSSPYPGMPVDSKFYKMIKEGFRMLSPEHAPAEM
YDIMKTCWDADPLKRPTFKQIVQLIEKQISESTNHIYSNLANCSPNRQKP
VVDHSVRINSVGSTASSSQPLLVHDDV

By “cluster of differentiation 117 (CD117; C-kit; SCFR) polynucleotide” is meant a nucleic acid molecule that encodes a CD117 polypeptide as well as the introns, exons, 3′ untranslated regions, 5′ untranslated regions, and regulatory sequences associated with its expression, or fragments thereof. In embodiments, a CD117 polynucleotide is the genomic sequence, cDNA, mRNA, or gene associated with and/or required for CD117 expression. An exemplary CD117 polynucleotide sequence from Homo sapiens is provided below (NCBI Ref. Seq. Accession No. NM_000222.2), and an exemplary CD117 gene sequence is provided at ENSEMBL Accession No. ENSG00000157404.

>NM_000222.2 Homo sapiens KIT proto-oncogene, receptor tyrosine
kinase (KIT), transcript variant 1, mRNA
(SEQ ID NO: 500)
TCTGGGGGCTCGGCTTTGCCGCGCTCGCTGCACTTGGGCGAGAGCTGGAACGTGGACCAGAGCT
CGGATCCCATCGCAGCTACCGCGATGAGAGGCGCTCGCGGCGCCTGGGATTTTCTCTGCGTTCT
GCTCCTACTGCTTCGCGTCCAGACAGGCTCTTCTCAACCATCTGTGAGTCCAGGGGAACCGTCT
CCACCATCCATCCATCCAGGAAAATCAGACTTAATAGTCCGCGTGGGCGACGAGATTAGGCTGT
TATGCACTGATCCGGGCTTTGTCAAATGGACTTTTGAGATCCTGGATGAAACGAATGAGAATAA
GCAGAATGAATGGATCACGGAAAAGGCAGAAGCCACCAACACCGGCAAATACACGTGCACCAAC
AAACACGGCTTAAGCAATTCCATTTATGTGTTTGTTAGAGATCCTGCCAAGCTTTTCCTTGTTG
ACCGCTCCTTGTATGGGAAAGAAGACAACGACACGCTGGTCCGCTGTCCTCTCACAGACCCAGA
AGTGACCAATTATTCCCTCAAGGGGTGCCAGGGGAAGCCTCTTCCCAAGGACTTGAGGTTTATT
CCTGACCCCAAGGCGGGCATCATGATCAAAAGTGTGAAACGCGCCTACCATCGGCTCTGTCTGC
ATTGTTCTGTGGACCAGGAGGGCAAGTCAGTGCTGTCGGAAAAATTCATCCTGAAAGTGAGGCC
AGCCTTCAAAGCTGTGCCTGTTGTGTCTGTGTCCAAAGCAAGCTATCTTCTTAGGGAAGGGGAA
GAATTCACAGTGACGTGCACAATAAAAGATGTGTCTAGTTCTGTGTACTCAACGTGGAAAAGAG
AAAACAGTCAGACTAAACTACAGGAGAAATATAATAGCTGGCATCACGGTGACTTCAATTATGA
ACGTCAGGCAACGTTGACTATCAGTTCAGCGAGAGTTAATGATTCTGGAGTGTTCATGTGTTAT
GCCAATAATACTTTTGGATCAGCAAATGTCACAACAACCTTGGAAGTAGTAGATAAAGGATTCA
TTAATATCTTCCCCATGATAAACACTACAGTATTTGTAAACGATGGAGAAAATGTAGATTTGAT
TGTTGAATATGAAGCATTCCCCAAACCTGAACACCAGCAGTGGATCTATATGAACAGAACCTTC
ACTGATAAATGGGAAGATTATCCCAAGTCTGAGAATGAAAGTAATATCAGATACGTAAGTGAAC
TTCATCTAACGAGATTAAAAGGCACCGAAGGAGGCACTTACACATTCCTAGTGTCCAATTCTGA
CGTCAATGCTGCCATAGCATTTAATGTTTATGTGAATACAAAACCAGAAATCCTGACTTACGAC
AGGCTCGTGAATGGCATGCTCCAATGTGTGGCAGCAGGATTCCCAGAGCCCACAATAGATTGGT
ATTTTTGTCCAGGAACTGAGCAGAGATGCTCTGCTTCTGTACTGCCAGTGGATGTGCAGACACT
AAACTCATCTGGGCCACCGTTTGGAAAGCTAGTGGTTCAGAGTTCTATAGATTCTAGTGCATTC
AAGCACAATGGCACGGTTGAATGTAAGGCTTACAACGATGTGGGCAAGACTTCTGCCTATTTTA
ACTTTGCATTTAAAGGTAACAACAAAGAGCAAATCCATCCCCACACCCTGTTCACTCCTTTGCT
GATTGGTTTCGTAATCGTAGCTGGCATGATGTGCATTATTGTGATGATTCTGACCTACAAATAT
TTACAGAAACCCATGTATGAAGTACAGTGGAAGGTTGTTGAGGAGATAAATGGAAACAATTATG
TTTACATAGACCCAACACAACTTCCTTATGATCACAAATGGGAGTTTCCCAGAAACAGGCTGAG
TTTTGGGAAAACCCTGGGTGCTGGAGCTTTCGGGAAGGTTGTTGAGGCAACTGCTTATGGCTTA
ATTAAGTCAGATGCGGCCATGACTGTCGCTGTAAAGATGCTCAAGCCGAGTGCCCATTTGACAG
AACGGGAAGCCCTCATGTCTGAACTCAAAGTCCTGAGTTACCTTGGTAATCACATGAATATTGT
GAATCTACTTGGAGCCTGCACCATTGGAGGGCCCACCCTGGTCATTACAGAATATTGTTGCTAT
GGTGATCTTTTGAATTTTTTGAGAAGAAAACGTGATTCATTTATTTGTTCAAAGCAGGAAGATC
ATGCAGAAGCTGCACTTTATAAGAATCTTCTGCATTCAAAGGAGTCTTCCTGCAGCGATAGTAC
TAATGAGTACATGGACATGAAACCTGGAGTTTCTTATGTTGTCCCAACCAAGGCCGACAAAAGG
AGATCTGTGAGAATAGGCTCATACATAGAAAGAGATGTGACTCCCGCCATCATGGAGGATGACG
AGTTGGCCCTAGACTTAGAAGACTTGCTGAGCTTTTCTTACCAGGTGGCAAAGGGCATGGCTTT
CCTCGCCTCCAAGAATTGTATTCACAGAGACTTGGCAGCCAGAAATATCCTCCTTACTCATGGT
CGGATCACAAAGATTTGTGATTTTGGTCTAGCCAGAGACATCAAGAATGATTCTAATTATGTGG
TTAAAGGAAACGCTCGACTACCTGTGAAGTGGATGGCACCTGAAAGCATTTTCAACTGTGTATA
CACGTTTGAAAGTGACGTCTGGTCCTATGGGATTTTTCTTTGGGAGCTGTTCTCTTTAGGAAGC
AGCCCCTATCCTGGAATGCCGGTCGATTCTAAGTTCTACAAGATGATCAAGGAAGGCTTCCGGA
TGCTCAGCCCTGAACACGCACCTGCTGAAATGTATGACATAATGAAGACTTGCTGGGATGCAGA
TCCCCTAAAAAGACCAACATTCAAGCAAATTGTTCAGCTAATTGAGAAGCAGATTTCAGAGAGC
ACCAATCATATTTACTCCAACTTAGCAAACTGCAGCCCCAACCGACAGAAGCCCGTGGTAGACC
ATTCTGTGCGGATCAATTCTGTCGGCAGCACCGCTTCCTCCTCCCAGCCTCTGCTTGTGCACGA
CGATGTCTGAGCAGAATCAGTGTTTGGGTCACCCCTCCAGGAATGATCTCTTCTTTTGGCTTCC
ATGATGGTTATTTTCTTTTCTTTCAACTTGCATCCAACTCCAGGATAGTGGGCACCCCACTGCA
ATCCTGTCTTTCTGAGCACACTTTAGTGGCCGATGATTTTTGTCATCAGCCACCATCCTATTGC
AAAGGTTCCAACTGTATATATTCCCAATAGCAACGTAGCTTCTACCATGAACAGAAAACATTCT
GATTTGGAAAAAGAGAGGGAGGTATGGACTGGGGGCCAGAGTCCTTTCCAAGGCTTCTCCAATT
CTGCCCAAAAATATGGTTGATAGTTTACCTGAATAAATGGTAGTAATCACAGTTGGCCTTCAGA
ACCATCCATAGTAGTATGATGATACAAGATTAGAAGCTGAAAACCTAAGTCCTTTATGTGGAAA
ACAGAACATCATTAGAACAAAGGACAGAGTATGAACACCTGGGCTTAAGAAATCTAGTATTTCA
TGCTGGGAATGAGACATAGGCCATGAAAAAAATGATCCCCAAGTGTGAACAAAAGATGCTCTTC
TGTGGACCACTGCATGAGCTTTTATACTACCGACCTGGTTTTTAAATAGAGTTTGCTATTAGAG
CATTGAATTGGAGAGAAGGCCTCCCTAGCCAGCACTTGTATATACGCATCTATAAATTGTCCGT
GTTCATACATTTGAGGGGAAAACACCATAAGGTTTCGTTTCTGTATACAACCCTGGCATTATGT
CCACTGTGTATAGAAGTAGATTAAGAGCCATATAAGTTTGAAGGAAACAGTTAATACCATTTTT
TAAGGAAACAATATAACCACAAAGCACAGTTTGAACAAAATCTCCTCTTTTAGCTGATGAACTT
ATTCTGTAGATTCTGTGGAACAAGCCTATCAGCTTCAGAATGGCATTGTACTCAATGGATTTGA
TGCTGTTTGACAAAGTTACTGATTCACTGCATGGCTCCCACAGGAGTGGGAAAACACTGCCATC
TTAGTTTGGATTCTTATGTAGCAGGAAATAAAGTATAGGTTTAGCCTCCTTCGCAGGCATGTCC
TGGACACCGGGCCAGTATCTATATATGTGTATGTACGTTTGTATGTGTGTAGACAAATATTTGG
AGGGGTATTTTTGCCCTGAGTCCAAGAGGGTCCTTTAGTACCTGAAAAGTAACTTGGCTTTCAT
TATTAGTACTGCTCTTGTTTCTTTTCACATAGCTGTCTAGAGTAGCTTACCAGAAGCTTCCATA
GTGGTGCAGAGGAAGTGGAAGGCATCAGTCCCTATGTATTTGCAGTTCACCTGCACTTAAGGCA
CTCTGTTATTTAGACTCATCTTACTGTACCTGTTCCTTAGACCTTCCATAATGCTACTGTCTCA
CTGAAACATTTAAATTTTACCCTTTAGACTGTAGCCTGGATATTATTCTTGTAGTTTACCTCTT
TAAAAACAAAACAAAACAAAACAAAAAACTCCCCTTCCTCACTGCCCAATATAAAAGGCAAATG
TGTACATGGCAGAGTTTGTGTGTTGTCTTGAAAGATTCAGGTATGTTGCCTTTATGGTTTCCCC
CTTCTACATTTCTTAGACTACATTTAGAGAACTGTGGCCGTTATCTGGAAGTAACCATTTGCAC
TGGAGTTCTATGCTCTCGCACCTTTCCAAAGTTAACAGATTTTGGGGTTGTGTTGTCACCCAAG
AGATTGTTGTTTGCCATACTTTGTCTGAAAAATTCCTTTGTGTTTCTATTGACTTCAATGATAG
TAAGAAAAGTGGTTGTTAGTTATAGATGTCTAGGTACTTCAGGGGCACTTCATTGAGAGTTTTG
TCTTGGATATTCTTGAAAGTTTATATTTTTATAATTTTTTCTTACATCAGATGTTTCTTTGCAG
TGGCTTAATGTTTGAAATTATTTTGTGGCTTTTTTTGTAAATATTGAAATGTAGCAATAATGTC
TTTTGAATATTCCCAAGCCCATGAGTCCTTGAAAATATTTTTTATATATACAGTAACTTTATGT
GTAAATACATAAGCGGCGTAAGTTTAAAGGATGTTGGTGTTCCACGTGTTTTATTCCTGTATGT
TGTCCAATTGTTGACAGTTCTGAAGAATTCTAATAAAATGTACATATATAAATCAAAAAAAAAA
AAAAAA

The term “linker”, as used herein, refers to a molecule that links two moieties. In one embodiment, the term “linker” refers to a covalent linker (e.g., covalent bond) or a non-covalent linker.

“Makassar” or “Hb G-Makassar” refers to a human β-hemoglobin variant, the human Hemoglobin (Hb) of G-Makassar variant or mutation (HB Makassar variant), which is an asymptomatic, naturally occurring variant (E6A) hemoglobin. Hb G-Makassar was first identified in Indonesia. (Mohamad, A. S. et al., 2018, Hematol. Rep., 10 (3): 7210 (doi: 10.4081/hr.2018.7210). The Hb G-Makassar mobility is slower when subjected to electrophoresis. The Makassar β-hemoglobin variant has its anatomical abnormality at the β-6 or A3 location where the glutamyl residue typically is replaced by an alanyl residue. The substitution of single amino acid in the gene encoding the β-globin subunit β-6 glutamyl to valine will result as sickle cell disease. Routine procedures, such as isoelectric focusing, hemoglobin electrophoresis separation by cation-exchange High Performance Liquid Chromatography (HPLC) and cellulose acetate electrophoresis, have been unable to separate the Hb G-Makassar and HbS globin forms, as they were found to have identical properties when analyzed by these methods. Consequently, Hb G-Makassar and HbS have been incorrectly identified and mistaken for each other by those skilled in the art, thus leading to misdiagnosis of Sickle Cell Disease (SCD). In one embodiment, the valine at amino acid position 6, which causes sickle cell disease, is replaced with an alanine, to thereby generate an Hb variant (Hb Makassar) that does not generate a sickle cell phenotype. In some embodiments, a Val→Ala (GTG→GCG) replacement (i.e., the Hb Makassar variant) can be generated using an A·T to G·C base editor (ABE).

Thus, the present disclosure includes compositions and methods for base editing a thymidine (T) to a cytidine (C) in the codon of the sixth amino acid of a sickle cell disease variant of the β-globin protein (Sickle HbS; E6V), thereby substituting an alanine for a valine (V6A) at this amino acid position. Substitution of alanine for valine at position 6 of HbS generates a β-globin protein variant that does not have a sickle cell phenotype (e.g., does not have the potential to polymerize as in the case of the pathogenic variant HbS). Accordingly, the compositions and methods of the disclosure are useful for the treatment of sickle cell disease (SCD).

By “marker” is meant any protein or polynucleotide having an alteration in expression, level, structure, or activity that is associated with a disease or disorder. In some cases, the disease or disorder is sickle cell disease. Non-limiting examples of markers include a Makassar variant of beta globin, beta globin, fetal hemoglobin, CD117, and variants of CD117 provided herein.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, IRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi: 10.1038/nbt.4172. In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
(SEQ ID NO: 196)
MDSLLMNRRKFLYQFKNVRWAKGRRETYLC,
(SEQ ID NO: 328)
PKKKRKVEGADKRTADGSEFESPKKKRKV,
(SEQ ID NO: 329)
RKSGKIAAIVVKRPRKPKKKRKV,
or
(SEQ ID NO: 196)
MDSLLMNRRKFLYQFKNVRWAKGRRETYLC.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, CsxIS, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363 (6422): 88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-230, and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase).

As used herein, “obtaining” as in “obtaining an agent” includes synthesizing, purchasing, or otherwise acquiring the agent.

By “Stem Cell Factor (SCF) polypeptide” is meant a polypeptide having at least about 85% amino acid sequence identity to an amino acid sequence provided at NCBI Ref. Seq. Accession No. NP_000890, reproduced below, or a fragment thereof that functions in hematopoiesis. In some embodiments, a SCF polypeptide or fragment thereof binds CD117.

>NP_000890.1 kit ligand isoform b precursor [Homo
sapiens]
(SEQ ID NO: 501)
MKKTQTWILTCIYLQLLLFNPLVKTEGICRNRVTNNVKDVTKLVANLPKD
YMITLKYVPGMDVLPSHCWISEMVVQLSDSLTDLLDKFSNISEGLSNYSI
IDKLVNIVDDLVECVKENSSKDLKKSFKSPEPRLFTPEEFFRIFNRSIDA
FKDFVVASETSDCVVSSTLSPEKDSRVSVTKPFMLPPVAASSLRNDSSSS
NRKAKNPPGDSSLHWAAMALPALFSLIIGFAFGALYWKKROPSLTRAVEN
IQINEEDNEISMLQEKEREFQEV.

By “stem cell factor (SCF) polynucleotide” is meant a nucleic acid molecule that encodes an SCF polypeptide as well as the introns, exons, 3′ untranslated regions, 5′ untranslated regions, and regulatory sequences associated with its expression, or fragments thereof. In embodiments, an SCF polynucleotide is the genomic sequence, cDNA, mRNA, or gene associated with and/or required for SCF expression. An exemplary SCF polynucleotide sequence from Homo sapiens is provided below (NCBI Ref. Seq. Accession No. NM_003994.5).

>NM_003994.5 Homo sapiens KIT ligand (KITLG), transcript variant
a, mRNA
(SEQ ID NO: 502)
GGGCTTCGCTCGCCGCCTCGCGCCGAGACTAGAAGCGCTGCGGGAAGCAGGGACAGTGGAGAGG
GCGCTGCGCTCGGGCTACCCAATGCGTGGACTATCTGCCGCCGCTGTTCGTGCAATATGCTGGA
GCTCCAGAACAGCTAAACGGAGTCGCCACACCACTGTTTGTGCTGGATCGCAGCGCTGCCTTTC
CTTATGAAGAAGACACAAACTTGGATTCTCACTTGCATTTATCTTCAGCTGCTCCTATTTAATC
CTCTCGTCAAAACTGAAGGGATCTGCAGGAATCGTGTGACTAATAATGTAAAAGACGTCACTAA
ATTGGTGGCAAATCTTCCAAAAGACTACATGATAACCCTCAAATATGTCCCCGGGATGGATGTT
TTGCCAAGTCATTGTTGGATAAGCGAGATGGTAGTACAATTGTCAGACAGCTTGACTGATCTTC
TGGACAAGTTTTCAAATATTTCTGAAGGCTTGAGTAATTATTCCATCATAGACAAACTTGTGAA
TATAGTGGATGACCTTGTGGAGTGCGTGAAAGAAAACTCATCTAAGGATCTAAAAAAATCATTC
AAGAGCCCAGAACCCAGGCTCTTTACTCCTGAAGAATTCTTTAGAATTTTTAATAGATCCATTG
ATGCCTTCAAGGACTTTGTAGTGGCATCTGAAACTAGTGATTGTGTGGTTTCTTCAACATTAAG
TCCTGAGAAAGGGAAGGCCAAAAATCCCCCTGGAGACTCCAGCCTACACTGGGCAGCCATGGCA
TTGCCAGCATTGTTTTCTCTTATAATTGGCTTTGCTTTTGGAGCCTTATACTGGAAGAAGAGAC
AGCCAAGTCTTACAAGGGCAGTTGAAAATATACAAATTAATGAAGAGGATAATGAGATAAGTAT
GTTGCAAGAGAAAGAGAGAGAGTTTCAAGAAGTGTAATTGTGGCTTGTATCAACACTGTTACTT
TCGTACATTGGCTGGTAACAGTTCATGTTTGCTTCATAAATGAAGCAGCTTTAAACAAATTCAT
ATTCTGTCTGGAGTGACAGACCACATCTTTATCTGTTCTTGCTACCCATGACTTTATATGGATG
ATTCAGAAATTGGAACAGAATGTTTTACTGTGAAACTGGCACTGAATTAATCATCTATAAAGAA
GAACTTGCATGGAGCAGGACTCTATTTTAAGGACTGCGGGACTTGGGTCTCATTTAGAACTTGC
AGCTGATGTTGGAAGAGAAAGCACGTGTCTCAGACTGCATGTACCATTTGCATGGCTCCAGAAA
TGTCTAAATGCTGAAAAAACACCTAGCTTTATTCTTCAGATACAAACTGCAGCCTGTAGTTATC
CTGGTCTCTGCAAGTAGATTTCAGCTTGGATAGTGAGGGTAACAATTTTTCTCAAAGGGATCTG
GAAAAAATGTTTAAAACTCAGTAGTGTCAGCCACTGTACAGTGTAGAAAGCAGTGGGAACTGTG
ATTGGATTTGGCAACATGTCAGCTTTATAGTTGCCGATTAGTGATATGGGTCTGATTTCGATCT
CTTCCTGATGTAAACCATGCTCACCCATATCCCACTATACAAATGCAAATGGTTGCCTGGTTCC
ATTTATGCAAGGGAGCCAGTACTGAATTATGCCTTGGCAGAGGGGAGACTCCAAAAGAGTCATC
GCAGGAAGAAGTTAAGAACACTGAACATCAGAACAGTCTGCCAAGAAGGACATTGGCATCCTGG
GAAAGTCCGCCTTTTCCCTTGACCACTATAGGGTGTATAAATCGTGTTTGCAAAATGTGTTATG
ATGTGTTTATATTCTAAAACTATTACAGAGCTATGTAAAGGGACTTAGGAGAAAATGCTGAATG
TAAGATGGTCCCATTTCAATTTCCACCATGGGAGAGCCTAAAAATAAATTATGACATTTAGTAT
CTAAGGTTAGAAAACCACGCCCACATGCTAATATGGGTGTTGAAAACTAGGTTACTTATAATGC
AAGGAATCAGGAAACTTTAGTTATTTATAGTATAATCACCATTATCTGTTTAAAGGATCCATTT
AGTTAAAATCGGGCACTCTATATTCATTAAGGTTTATGAATTAAAAAGAAAGCTTTATGTAGTT
ATGCATGTCAGTTTGCTATTTAAAATGTGTGACAGTGTTTGTCATATTAAGAGTGAATTTGGCA
GGAATTCCCAAGATGGACATTGTGCTTTTAAACTAGAACTTGTAAGACATTATGTGAATATCCC
TTGCCAATTTTTTTTATAATAAGAAAACATCTGACTAAAGTCAAAGAATGATTTCTTATGGTTT
ATTTTGATGAAAGTTCTTTTAACATGTCTTGAATGTACACATAAAGGAATCCAAAGCTTTCCAT
TCTAACTTAATCTTTGTGATAACATTATTGCCATGTTCTACAACCGTAAGATGACAGTTTTCAA
TGTAGTGACACAAAAGGGCATGAAAAACTAACTGCTAGCTTTCCTTTCATTTCAAAAGTCCAAG
AATTTCTAGTATATTTGGATTTTAGCTTCTGTTCAAAGCAAATCCAGATGCAACTCCAGTAAGT
GGCCTTTGCTCTTTTTTGTACCAAAGAGCCCAGATGATTCCTACAGTCCCTTTCTTCTCTAACA
TGCTGTGGTTCCTTAAATATGAGTAATTTCTCTAAGATATAACCCAGGTGCTTTGAGAAGCTGC
ATTAAGGTGTTCAGGCCCTCAGATATCACATGGTACACTTGATTAGTAATAAAACCAGAGATCA
ATTTAAATTGCTGATAGGTCCTGTCTCAGTGTGTGGCATTGACTGTTTTCAGGAAAATAGATAC
AGATTAATATGAGTTATGCGTGTAGGTTGTGTATAGATTGAGAAGATAGATACTTCTCAATCTA
GTAGTTTGATTTATTTAACCAATGGTTTCAGTTTGCTTGAGCATATGAAAATCCTGCTTAATGT
GCTTAAGAGTATAATAAATGTGTACTTTTGTCCTCAAACCTAGTAGCTGGGTTTTAACACTCAT
GGACATGGTCTTAATCAATGGAGTTAAATAAACAAATTCAGCAAGTTATTAAATCTGACATGGT
AGGAGAGGGGAGATGTGTCCTGCTTATTAAATGTGTTGGTCCATTGAAAGTTACATGGATTGCC
AATTTTTAAAACACTAAAGTTGAATAAAATGCATGAACAATAGAAAAATGCTGAACATTATTTT
GGATGCTAGCTGCTTGGACATTAACTGTGTTATTTCTGCTTTGAGATGAAAATATATATTTATC
TTTGCTTATTTTATCCCAGATGTGTTCTGAATATCCTTCTTCATAAATCATGGAAAACTCACTG
CTGAGATAGTAAACCATGAAATCGCCTTTTCAGTTGGTGCCATGTATCTGACAGTTCCATCTTG
GAAGGTTTCAAAATTACCTTTTAAAATGATCTCAGAAGTCTGTAGATTCTCAATGATACTGAAA
GCTTTGCACCTCTTTGGTAGAAACCAGGTCTATTTAGAAAATGGCTTTATGATAAATGTTGCCT
CCTGAGTGATAATGAAGTGTTCCTGGATATTGTATTGTAATTTAATGTGCTTACCACACTGCCA
CATTTTAATGAGTCAGAGAAAAATTAATTTTTCTTCAATACAATAATAGAACAAGTAGCCTATT
CTCTTAAAAAGTATGTGAAAAGAAAATTATGAAAAAATATGCATACCTAATGAAGTATTGGTTT
TAGTAAGAATTAAATACATTTCATTGAGCTTTAAAGTACTTTGGAGAAACTTTGGGGCACGTTT
TCCTACTCTAATTCAACTAAAGTTATAAATAAAGAGAAAAACTCATTCAGAAATCATGGATTTT
AAAAATATTTTACTGCAGCCAAGTTTTCATTTCAAAATGTAATTTCAGTTTGGAGCTTTTAGGC
ATTATGTATATTTAAAAAATATATTCTTCAAAAATGCATTTTGGCATGGTGGGATGGATGTTGC
AAAAGATATCCGGAGCCTCCAGTCTGTCATTAACTGATATGGTAAATCACCTCTCTTCTTTGGG
TCTCAATTTTTTATTTATCTATATGGTAAACTCAGAGATCACTCCTTAGGGGTGAGTCCTATTG
CAATATGACCGACAAAGAAGACAAAATAGCATTGAAACTAACCCATACAAAATATCCAACTCTG
GATTCTGTGAATAAGTATCTTGACCATAAAAAGTCATTGCTGTTCTTGTTTCTAATGTAAATAG
TGTCCATTAGTAAAAGTGAAATTCAGTCTTAAGTAGGGTGAATTGGATCACCATTTACACAAGA
GATGGCTTTTTCCTTTGCTTGAATAAACATTTTGGATCACCTCCAAAGAATGAAAACCAGTAGT
ACGTTTTAGTCATATTAGTCAGGATGAGAAACTATAAGATGTGTGTAACATTTGGAAATGCACC
AAAGTGAGCGTTTAAATCTTCTCATTTTATTGAAAACTAAGAGCAGAAAATGTAAAATGCTCAT
GAAGGTTTTGAATGCCAAAAGATATTTTAGAATCAATTTATAAAGGGGTAATTCATTAATTACA
CTTTAAAATTGGAAAGTGGGATAAGAAATCTAAAGTAAACCAGCTTATCTTTGAAACAATATTA
TTTTGAAATTGGCTTTAAAATAAAACCATTCAGATTGAAATTCTAATTAGCTCATTTGTGGAGT
TTGATCACACAATTCATAATGTTGCTGCTTTCCATTAACTAGTCTTGAAATGCCTTTGTTTGTA
AAAATAAAATAATGGTACTTTCATTTTATAACAAGGTGTTTTTTTCAAGAAATAATCCATGCTA
AAATGGATATTTGTGATCCTGAAATGTTTACTAAGCATTGTAAATTTATTTATAACTGCCATCT
CCAACTACATCCTTATGATGTTTTTAACAATAAAATTAAAACAACTGTTAAACTAAAAACCACA
CCGTTTTCCAGTACTTGATCTCTGAGCTACAATACTCACTAAATATAATTTTCCAATCAAAATA
TTCTATTCTATATTCTAAGGGTTAATATGTGATTATAGTGTCCACTTGCCACCATTTTTTTAAA
TCAATGGACTTGAAAAGTATTAATTTAGATGGATGCGCAGATATACCCTCAGTTCAGTCATAGA
TTGGAGTTTGCATATAATAATGTAAATGTATGTCGACACTATTCTAAATAGTTCTATTATGACT
GAAATTTAATTAAATAAAAAAGGTTGTAAAATGTGATGTGTATGTGTATATACTGTATGTGTAC
TTTTTAAAATAGGTGTATGTCCCAACCCTTTTTTATACAGGTTTGAATTTAAAATTACATGATA
TATACATATACTTTATTGTTCTAAATAAAGAATTTTATGCACTCTCAAAAAAAAAAAAAAAAAA.

By “subject” or “patient” is meant a mammal. Non-limiting examples of mammals include a primate (e.g., a human or a cynomolgus monkey) or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates (e.g., a cynomolgus monkey), cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

As used herein, the term “human antibody” refers to an antibody in which substantially every part of the protein (e.g., CDR, framework, C_{L}, C_{H} domains (e.g., C_{H1}, C_{H2}, C_{H3}), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell (e.g., yeast) that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)2 or other target-binding subdomains of antibodies) which contain minimal sequences derived from non-human immunoglobulin. In general, the humanized antibody will comprise substantially all of at least one, and typically two, variable regions, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and 6,180,370 to Queen et al; EP239400; PCT publication WO 91/09967; U.S. Pat. No. 5,225,539; EP592106; and EP519596; incorporated herein by reference.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild type or healthy cell. In other embodiments and without limitation, a reference is an untreated cell that is not subjected to a test condition, or is subjected to placebo or normal saline, medium, buffer, and/or a control vector that does not harbor a polynucleotide of interest. In some cases, a “reference” is a n untreated subject, such as a subject not administered a hematopoietic stem cell edited according to the methods of the present disclosure. In some cases, the subject is a healthy subject (e.g., a subject not having sickle cell disease). In some embodiments, the reference is an unedited or wild type cell, polypeptide, or polynucleotide.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild type sequence of a protein of interest. In other embodiments, a reference sequence is a polynucleotide sequence encoding a wild type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease: RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), or derivatives thereof (e.g., a sequence with at least about 85% sequence identity to a Cas9, such as Nme2Cas9 or spCas9).

As used herein, the term “scFv” refers to a single chain Fv antibody in which the variable regions of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

Amino acids generally can be grouped according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

By “selectively binds” is meant specifically binds a wild type version of the cell surface protein but exhibits reduced binding or fails to detectably bind to the cell surface protein comprising a mutation. In embodiments, an antibody of the present disclosure selectively binds to a wild type CD117 polypeptide but exhibits reduced binding to a CD117 polypeptide comprising one or more amino acid alterations, such as those provided herein, relative to the wild type CD117 polypeptide. In embodiments, an antibody of the present disclosure binds a wild type CD117 polypeptide 1.1-fold, 1.2-fold, 1.3-fold, 1.4-fold, 1-fold, 5-fold, 1.75-fold, 2-fold, 3-fold, 4-fold, 5-fold, 6-fold, 7-fold, 8-fold, 9-fold, 10-fold, 100-fold, 1000-fold, 10000-fold, 100000-fold, or 1000000-fold more strongly (e.g., as quantified using KD(M), where a lower KD(M) indicates stronger binding) than to an altered CD117 polypeptide of the present disclosure.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g., a Cas9) or a base editor.

The term “target site” refers to a sequence within a nucleic acid molecule that is modified. In embodiments, the modification is deamination of a base. The deaminase can be an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a compositions as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA glycosylase
inhibitor
(SEQ ID NO: 231)
MTNLSDIIEKETGKQLVIQESILMLPEEVEEVIGNKPESDILVHTAYDES
TDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “transfecting” or “transfection” is used synonymously and according to some aspects and embodiments herein means the introduction of heterologous nucleic acid (DNA/RNA) into a eukaryotic cell, in particular yeast cells.

According to some aspects and embodiments herein, antibody fragments are understood as meaning functional parts of antibodies, such as Fc, Fab, Fab′, Fv, F(ab′)2, scFv. According to some aspects and embodiments herein, corresponding biologically active fragments are to be understood as meaning those parts of antibodies which are capable of binding to an antigen, such as Fab, Fab′, Fv, F(ab′)2, and scFv.

As used herein, the term “vector” refers to a means of introducing a nucleic acid sequence into a cell, resulting in a transformed cell. Vectors include plasmids, transposons, phages, viruses, liposomes, lipid nanoparticles, and episomes. “Expression vectors” are nucleic acid sequences comprising the nucleotide sequence to be expressed in the recipient cell. Expression vectors contain a polynucleotide sequence as well as additional nucleic acid sequences to promote and/or facilitate the expression of the introduced sequence, such as start, stop, enhancer, promoter, and secretion sequences, into the genome of a mammalian cell. Examples of vectors include nucleic acid vectors, e.g., DNA vectors, such as plasmids, RNA vectors, viruses or other suitable replicons (e.g., viral vectors). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Certain vectors that can be used for the expression of antibodies, antibody fragments, base editors, guide polynucleotides, and/or base editor systems of some aspects and embodiments herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of some aspects and embodiments herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIGS. 1A and 1B provide schematics showing an engineered stem cell antibody paired evasion approach (ESCAPE). FIG. 1A provides a schematic showing conditioning with a mAb. A bone marrow niche may be depleted with mAb interfering with an essential protein expressed on the surface of stem cells. Following marrow clearance, hematopoietic stem cells (HSCs) containing mutations in the mAb-targeted protein that make the cells resistant toward mAb ablation can be supplied for engraftment. In some cases, the method involves administering the antibody more than once (i.e., re-dosing). FIG. 1B provides a schematic showing selective enrichment of engineered hematopoietic stem cells (eHSC) harboring mutations in an essential cell surface protein. (e.g., CD117). HSC can be engineered, through base editing, to contain non-synonymous mutations that do not inhibit normal cellular function and resist mAb ablation. Unedited or endogenous cells remaining in the niche can be selectively eliminated due to sensitivity to mAb binding and interference.

FIG. 2A-2D provide schematics showing an engineered stem cell antibody paired evasion (ESCAPE) approach to non-genotoxic cell conditioning. FIG. 2A provides a schematic showing how base editing can be used to generate cells having increased fetal hemoglobin production (HbF) and an altered CD117 (c-KIT) polypeptide that has reduced binding to an antibody (e.g., a non-genotoxic stem cell factor-(SCF-)blocking antibody). In embodiments, cell conditioning strategy eliminates the need for busulfan conditioning by replacing the chemotherapy agent with a non-genotoxic SCF-blocking antibody. The strategy includes modifying the CD117 (c-KIT) antigen by creating a single or multiple-nucleotide polymorphism (SNP or nucleotide alteration) through base editing. In embodiments, the SNP in c-KIT (e.g., a non-synonymous mutation) prevents or reduces antibody (Ab) (e.g., ABTx052) binding. In embodiments, the modification to the CD117 antigen is multiplexed with an edit to a sickle cell disease (SCD) target (e.g., an edit associated with an upregulation in fetal hemoglobin (HbF) expression, such as an edit to a promoter region). In some cases, both edits are carried out using the same adenosine base editor (ABE) in combination with two or more guide RNAs (gRNAs) (e.g., sgRNA_015 and a CD117-targeting gRNA). In various instances, the edited cells are derived from a patient to which they are to be administered. FIGS. 2B and 2C provide schematics showing how combining an SCF-blocking antibody that is specific for wild type c-KIT antigen will ablate cells that are displaying wild type c-KIT. Therefore, ESCAPE enables the selective enrichment of c-KIT edited cells. FIG. 2C provides a schematic showing that unedited cells are depleted when an SCF-blocking antibody is present. In embodiments, cells that contain a modified c-KIT polypeptide are not depleted by the SCF-blocking antibody and repopulate and engraft. Multiplex-edited cells (i.e., cells with an edit for treatment of sickle cell disease and an edit to the CD117 antigen) are administered in embodiments concurrently with, after, or prior to administration of an SCF-blocking antibody, where the administration of the antibody is associated with enrichment of the edited cells in a subject. FIG. 2D provides a schematic showing how alteration of a CD117 epitope encoded by an HSC using base editing allows the HSC to selectively evade being bound by an antibody that binds the CD117 epitope.

FIG. 3 provides a schematic summarizing screens that were completed to identify ABE8.8-compatible guides (left panel) for use in introducing alterations to CD117 antigens and antibodies showing reduced binding to the altered antigens (right panel) and suitable for use in the ESCAPE approach to non-genotoxic cell conditioning. The guides identified in the screens included CC128, CC79, and CC89. The antibodies identified in the screens included ABTx052, ABTx062, ABTx025, ABTx030, ABTx070, and ABTx071. As shown in the left panel of FIG. 3, 102 guide RNAs capable of installing missense mutations were computationally identified. Among these, 27 gRNAs were selected based on highest editing efficiencies in HEK293 cells. 8 gRNAs were selected based on high editing efficiencies in CD34+ cells. CD117 mutations produced by these gRNAs were characterized and 5 were chosen based on retention of normal ligand binding and phosphorylation capability in vitro. Also, as shown in the right panel of FIG. 3, 188 mAb clones were identified among which 72 clones had unique variable heavy chains (VH). 66 of these clones were screened for binding to wild type and variant CD117 proteins. 6 such antibodies were selected based on binding to wild type CD117 and lack of binding to variant CD117. One of the lead antibodies blocked CD117 binding to its natural ligand SCF and did not bind to one of the lead edits.

FIG. 4 provides crystallographic images of CD117 showing the location of amino acid residue alterations of the present disclosure. The alterations included T144A (sgRNACC89); Y249C and N260D (sgRNA CC128); and M351T (sgRNA CC79). The edits Y259C and N260D were located near the stem cell factor (SCF) binding site. In FIG. 4 the following alteration locations are highlighted in the structural images from top-to-bottom: Highlighted sites top to bottom: (CC89) T144A; (CC128) Y259C N260D; (CC79) M351T; and (CC78) Y418C. The protospacer corresponding to the guide RNA CC128 is provided in the figure and corresponds to SEQ ID NO: 830.

FIGS. 5A-5F provide plots and a histogram showing that ABTx052 lacks binding to CD117 edited using the guide CC128 and ABE8.8 (CC128-edited CD117), and blocks stem cell factor (SCF) binding to wild type CD117. FIG. 5A provides a plot showing biolayer interferometry (BLI) measurements demonstrating that ABTx052 (mAB-7) did not bind to the CC128-edited CD117 as purified protein. ABTx052 did bind with wtCD117 expressed on M07e cells with high affinity (20 μM). ABTx052 rapidly dissociated from CC128-edited CD117. The monoclonal antibody mAb-7 bound wtCD117 with a high affinity (KD<1E-12) but bound minimally to CC128-edited CD117. FIGS. 5B and 5F provide a plots showing that ABTx052 (mAB-7) blocked SCF binding to CD117. FIG. 5C provides a histogram showing that ABTx052 did not bind to the CC128-edited CD117 as expressed in M07e cells. FIG. 5D provides a plot showing that ABTx052 had an EC50 of about 20 μM. The vertical lines in FIGS. 5A and 5B indicate times of substrate addition (e.g., ABTx052 or SCF addition). FIG. 5E provides a plot showing flow cytometry data demonstrating that unedited CD34+ cells were bound highly (EC50 0.02 nM) by mAb-7 modified ton include a LALADA Fc alteration (i.e., ABTx135), while CD34+ cells edited with CD117 sgRNA showed minimal binding by the antibody. ABTx135 showed minimal binding to CD34+ cells expressing a CD117 variant prepared using guide CC128. 100,000 Unedited or CD117-edited human CD34+ hematopoietic stem and progenitor cells were incubated with varying concentrations of ABTx135, which corresponded to ABTx052 containing an LALADA alteration in the Fc domain, for 20 minutes at 4C in 100 μL total staining volume in PBS+2.5% FBS. After 20 minutes the cells were pelleted by centrifugation at 500×G for 5 minutes at 4 C. Supernatant was removed, and cells were washed 2 times in PBS+2.5% FBS. After the 2 washes, 100 μL of the secondary detection antibody at concentration of 25 μg/mL was added to the cell pellet. Cells were incubated with the secondary antibody for 20 minutes at 4 C. Cells were then washed 2 times with PBS+2.5% FBS at 4 C. Finally, cells were suspended in 100 μL of PBS+2.5% FBS and analyzed using flow cytometry. Geometric mean fluorescence intensities of staining were plotted (y-axis) with log of ABTx135 concentration (x-axis). Staining of cells with secondary antibody alone (dotted line) was used as negative control. Flow cytometry showed binding of mAb-7 to unedited CD34+ cells and lack of binding to CD117-edited CD34+ cells. Secondary antibody details: Goat anti-Human IgG Fc Cross-Adsorbed Secondary Antibody, DyLight 650, ThermoFisher, Cat #SA5-10137.

FIGS. 6A and 6B provide an SDS-PAGE gel image and a size-exclusion chromatography plot, respectively, showing the purity of the ABTx052 antibody.

FIGS. 7A and 7B provide plots showing the in vitro evaluation of mAb binding at increasing doses between unedited human hematopoietic stem cells (HSCs) and HSCs base edited to contain mutations in CD117 to block mAb binding. Human CD34+ cells edited with ABE8.8 and CD117-targeting guide RNA escaped recognition by the antibodies ABTx062 and ABTx052 that bind to unedited human HSCs expressing wild type CD117. The mAbs ABTx062 and ABTx052 showed loss of binding to CD34+ hematopoietic stem and progenitor cells (HSPCs; HSCs) edited using gRNA CC128. Edited cells expressed CD117*Y259C/N260D. The desired nucleotide edits caried out using the CC128 guide were 5G+7G.

FIGS. 8A to 8G provide plots and a bar graph showing that the CC128 engineered CD117 epitope was protective against ligand blocking by ABTx052 in vitro. FIGS. 8A and 8B provide plots showing that at Days 2 and 5 post-transfection, cells edited using the CC128 guide showed improved viability in the presence of ABTx052 relative to unedited cells. Cell viability of primary CD34+ derived edited cells with CC128 guide alone cultured in the presence (and absence of stem cell factor (SCF)) at increasing concentrations of ABTx052 Ab was preserved. Unedited cells exposed to ABTx052 exhibited viability that resembled complete withdrawal of SCF (dotted line). FIG. 8C shows that cells containing the target 5G+7G dual nucleotide edit combination introduced using the CC128 guide were enriched for in a 1:1 mix of edited and unedited cells over time in the presence of ABTx052. Next-generation sequencing (NGS) of the 1:1 mixes was used to show the enrichment by demonstrating an increase in editing frequency.

FIGS. 8D and 8E provide plots showing that contacting cells expressing a wild type CD117 polypeptide (“unedited cells”) with ABTx052 mimicked complete SCF withdrawal and led to ˜85% loss of viability in vitro, while edited cells showed increased viability relative to the unedited cells. Unedited, CD117-edited, and a 1:1 mixture of unedited and CD117-edited CD34+ cells were cultured in presence of varying concentrations of mAb-7 for 7 days. mAb-7 treatment depleted unedited cells to the level of complete SCF starvation (dotted line), while CD117-edited cells retained viability. Cell viability levels for the mixed population maintained an intermediate value. FIG. 8F provides a bar graph showing enrichment for base-edited cells containing a CC128 engineered CD117 epitope in in a co-culture of edited and non-edited cells contacted with concentrations of mAb-7 ranging from 100 ng/mL to 10000 ng/mL. After 7-days of co-culture in the presence of absence of mAb-7, genomic DNA was isolated from the co-cultures and subjected to next generation sequencing (NGS). NGS showed enrichment of CD117 editing in the treated cells compared with an untreated control. FIG. 8G provides a plot showing that mAb-7 selectively depleted unedited hematopoietic stem cells while cells expressing an engineered CD117 (engineered through base editing using the guide CC128) retained viability in vitro. In FIG. 8G, the dotted horizontal line represents cell viability corresponding to complete SCF starvation, and the control corresponded to cells that were not treated with mAb-7.

FIG. 9 provides a schematic diagram showing the design of an in vivo study to determine if CD117 edited cells had a similar function as wild type CD117 cells. Unedited or CD117-edited CD34+ cells were transplanted into NBSGW mice. Mouse bone marrow was harvested and subjected to flow cytometry and NGS analyses 16 weeks post transplantation. Marrow hematopoietic compartments were sorted.

FIGS. 10A and 10B provide a bar graph and a flow cytometry plot showing that engraftment of CD117 variants prepared using the guides CC79, CC84, CC89, CC90, CC119, and CC128 was not altered by base editing, as measured after an 8-week engraftment using flow cytometry. FIG. 10B provides a representative flow cytometry plot demonstrating engraftment of edited human CD45+ cells (hCD45+) in a mouse administered the cells. In FIG. 10B unedited mouse CD45+ cells are represented by the term “mCD45+.”

FIGS. 11A and 11B provide stacked bar graphs showing that CD117 edits were retained in the bulk bone marrow at the 8-week time point of engraftment. FIG. 11A shows percent editing 48 hours post electroporation (EP) in cells edited using ABE8.8 and the guides CC79, CC84, CC89, CC90, CC119, and CC128. The edits were 5G, 6G, 7G, 8G, 5G+7G, 6G+7G, and 6G+8G. FIG. 11B shows bulk bone marrow (BM) editing measured after 8 weeks of engraftment. In FIG. 11A, going from top-to-bottom of each stacked bar and from left to right, the edits represented in each bar are, where each bar is separated by a semicolon: 6G+8G, 8G; 6G+8G, 6G; 5G; 6G+7G, 7G, 6G; 5G; 5G+7G, 7G, 5G; and 7G. In FIG. 11B, going from top-to-bottom of each stacked bar and from left to right, the edits represented in each bar are, where each bar is separated by a semicolon: 6G+8G, 8G, 6G; 6G+8G, 6G; 5G; 6G+7G, 7G, 6G; 5G; 5G+7G, 5G; and 7G.

FIG. 12 provides a schematic showing that multiplex editing advantageously preserved therapeutic editing levels in hematopoietic stem cells (HSCs) and what the desirable multiplex edit (i.e., “Multiplex edited HSCs) was. In an embodiment, cells are multiplex edited using ABE8.8 in combination with a guide RNA for introducing an edit to a CD117 polynucleotide and an sgRNA_015 guide (HPFH edit). The guide sgRNA_015 was used to introduce a therapeutic edit to the cells and targeted the HBG1/2 promoter, and the sgRNA CC128 was used to introduce a conditioning edit to the cells and targeted a CD117 polynucleotide. In FIG. 12, SCF is represented by a filled-in grey circle, and +BEAM-101 gRNA indicates sgRNA_015.

FIGS. 13A and 13B provide stacked bar graphs showing that multiplex editing with sgRNA HBG1/2a-114 (sgRNA_015) and gRNA CC128 yielded highly efficient A: T to G: C base editing. FIG. 13A provides a stacked bar graph showing editing rates at the CD117 site targeted by the CC128 guide. FIG. 13B provides a stacked bar graph showing editing rates at the HBG1/2a-114 site targeted by sgRNA_015.

FIG. 14 provides a schematic showing a strategy for generating single cell clones for de-risking individual CC128 edits.

FIGS. 15A and 15B provide bar graphs showing editing rates for M07e clones and staining of the M07e clones with the antibody ABTx052. FIG. 15A provides a stacked bar graph showing allelic editing in the indicated clones. Going from top-to-bottom and from left-to-right, the edits depicted in each bar, where each bar is separated by a semicolon, are: 7G, 5G+7G, 5G; 7G, 5G+7G, 5G; 7G, 5G+7G, 5G; 7G, 5G+7G, 5G; 5G; 5G; 7G, 5G+7G+10G, 5G+7G, 5G; 7G, 5G+7G, 5G; and 7G, 5G+7G (where the location of the nucleotide edits are indicated by subscripts within the following sequence: 5′-AAATA₅TA₇ATA₁₀GCTGGCATCA-3′(SEQ ID NO: 830)). FIG. 15B provides a bar graph showing that some of the M07e clones showed reduced ABTx052 staining. The genotypes of the clones indicated on the x-axis of FIGS. 15A and 15B are provided in Table 12.

FIGS. 16A-16D provide Western blot images and a plot showing that Y259C (5G) and Y259C+N260D (5G+7G) CD117 mono-allelically edited cells could bind ligand (stem cell factor (SCF)) and induce phosphorylation in the presence of ABTx052 and that, therefore, the edited CD117 polypeptides were functional. FIG. 16A provides a Western blot image demonstrating phosphorylation of CD117 in the indicated clones (see Table 12 for a description of the clone allelic editing compositions). Cells were incubated with 1 μg/mL of ABTx052 antibody for 5 min before addition of SCF at 100 ng/ml for 10 min. Phospho CD117 was probed with anti-phospho CD117 ab Y719. FIG. 16B provides a plot showing how cells edited using the guide CC128 show increased viability in the presence of ABTx052 at the concentrations indicated on the x-axis and 100 ng/mL SCF relative to unedited (UN) cells under the same conditions. The monoclonal antibody ABTx052 (mAbABTx052) reduced cell proliferation of unedited cells by blocking c-KIT binding with SCF. Distinct differences in cell survival were observed between unedited and CC128 variant primary hematopoietic stem cells (HSCs) upon culturing with ABTx052. CC128-edited cells displayed higher survival relative to unedited cells. FIG. 16C provides a Western blot image showing phosphorylation of CD117 in cells edited using the indicated guides (CC128 or CC295) in combination with ABE8.8. The Western blot also shows that ABTx052 did not block SCF binding to the CD117 polypeptide altered using the CC128 guide. The cells were M07e cells. Cells were stimulated with 100 ng/mL SCF for 10 minutes. The c-KIT was probed using mAB 332. Phospho-c-KIT was probed using mAb Y719. CD117 edited using the guide CC128 was phosphorylated upon SCF stimulation. ABTx052 blocked phosphorylation of unedited c-KIT polypeptides. FIG. 16D provides a Western blot image showing results from an experiment in which M07e cell lines that were unedited, base edited with CD117-sgRNA, or CD117-knockout (KO) were treated with 100 ng/mL SCF in presence or absence of mAb-7. Phosphorylated CD117 was probed with anti-phospho CD117 mAb Y719. Cells expressing wild type CD117 underwent phosphorylation upon SCF stimulation, as did cells containing CD117 mono-allelic and bi-allelic edits. mAb-7 inhibited phosphorylation of WT CD117. Bi-allelically edited cells underwent normal levels of phosphorylation even in the presence of mAb-7. mAb-7 reduced but did not completely inhibit CD117 phosphorylation in cells containing a mono-allelic CD117 edit. CD117-KO cells served as a negative control.

FIG. 17 provides a schematic depicting a screen completed to identify new guides for use with an ABE-NRCH non-G PAM editor to alter a polynucleotide encoding a CD117 polypeptide. The criteria for selection from guide screening in HEK293 T cells were the following: 1) intended edit achieved at a frequency >25% of good next-generation sequencing quality, 2) low heterogeneity in final protein variant being generated, 3) conservation of protein sequence between cynomolgus genome (required at amino acid being targeted and preferred if 100% conserved).

FIGS. 18A and 18B provide flow cytometry histograms. FIG. 18A provides overlayed flow cytometry histograms showing that overall CD117 expression in cells remained constant following editing using the guides gRNA931 (CC200) and CC128. Expression was measured using the monoclonal antibody (mAb) 104D2. FIG. 18B provides overlayed flow cytometry histograms showing that CD34+ cells edited using gRNA 931 lacked binding to ABTx052.

FIG. 19 provides a plot showing that the CD117 S261G engineered epitope was protective against ligand blocking by ABTx052 in vitro. Cells edited using gRNA931 (CC200) showed increased viability relative to unedited cells when contacted with ABTx052 at the concentrations indicated on the x-axis.

FIGS. 20A-20H provide bar graphs showing percent human CD34+ (FIG. 20A), CD15+ (FIG. 20B), CD19+ (FIG. 20C), Lin−CD34+ (FIG. 20D), CD3+ (FIG. 20E), hCD33+SSC-Alow (FIG. 20F), GlyA (FIG. 20G), and CD33+SSC-Ahi (FIG. 20H) cells in mice following 8 weeks of engraftment with cells edited using the guides indicated on the X-axis (CC79, CC84, CC90, CC119, and CC128).

FIGS. 21A and 21B provide a stacked bar graph and a bar graph demonstrating highly efficient, bi-allelic editing was achieved in CD34+ cells and that the cells had normal colony forming unit (CFU) capacity. FIG. 21A provides a stacked bar graph showing that cells edited using the indicated guides (CC79, CC128, CC84, CC90, CC89, and CC119) had normal colony forming unity capacity, which was consistent with CD117 function being preserved, and that disrupting CD117 function (e.g., by editing using the guides 291 or 295) adversely impacted the colony forming ability of the edited cells. Disrupting CD117 function impacted the colony forming ability of the erythroid population. CFU assays indicated that editing c-KIT had minimal effect on myeloid colony formation. FIG. 21B provides a bar graph showing percent A>G editing achieved using the indicated guides (CC79, CC128, CC84, CC90, CC89, and CC119) after 24 hours (first bar from the left in each set of 3), 48 hours (second bar from the left in each set of 3), and 120 hours (3rd bar from the left in each set of 3). Over 85% bi-allelic editing was achieved.

FIGS. 22A-22D provide bar graphs demonstrating that c-KIT knock-out profoundly affected erythroid in vitro differentiation. FIG. 22A shows the total number of burst forming unit-erythroid cells (BFU-E) measured after cells were edited using the indicated guides (HFPH (sgRNA_015), CC119, CC126, CC290, CC291, CC292, CC293, CC294, and CC295). FIG. 22B shows the number of granulocyte-macrophage progenitor (GMP) colony forming units (CFU-GM) measured after cells were edited using the indicated guides (HFPH (sgRNA_015), CC119, CC126, CC290, CC291, CC292, CC293, CC294, and CC295). FIGS. 22C and 22D show levels of in vitro differentiation and myeloid 7-days post-transfection using the indicated editors. In FIGS. 22C and 22D, the bars from left-to-right correspond to the following: Unedited, HPFH (sgRNA_015), CC119, CC126, CC290, CC291, CC292, CC293, CC294, and CC295. Knockout edits results in profound erythroid in vitro differentiation defect and less profound myeloid differentiation defect.

FIGS. 23A and 23B provide stacked bar plots and bar plots showing results relating to editing of CD34+HSPCs to install c-KIT mutations. FIG. 23A provides a stacked bar plot showing editing efficiency with selected c-KIT guides (CC79, CC84, CC89, CC90, CC119, and CC128). Highly efficient editing in CD34+HSPCs was achieved and lead to expression of mutant c-KIT polypeptides in edited cells. FIG. 23B provides a bar graph showing that knocking out c-KIT (using guides CC291 and CC295) disrupted erythroid differentiation in vitro, but that editing using the guides HPFH (sgRNA_015), CC78, CC79, CC94, CC89, CC90, CC119, CC128, or CC84+CC90 did not. Mutations to c-KIT were selected that did not disrupt HSPC function and differentiation.

FIG. 24 provides a Western blot showing that the guides 291 and 295 used in combination with ABE8.8 were useful in successfully knocking out expression of CD117. Unedited & edited CD34 cells were stimulated with stem cell factor (SCF) for 10 mins and the cell lysate was subjected to a western blot using anti-CD117 antibody (MAB332) and anti-phospho CD117 antibody (Y719). Editing CD34+HSCs with KO sgRNA 291 & 295 and ABE8.8 mRNA displayed no CD117 phosphorylation.

FIG. 25 provides fluorescent images of cells showing that CD117 altered using the guide CC128 was internalized upon binding to stem cell factor (SCF), which is consistent with the altered CD117 polypeptide maintaining its function. SCF was conjugated with the pH-sensitive dye pHrodo-green. pHrodo-green fluoresces only when it enters a cell. Measurement of pHrodo-green fluorescence indicates internalization of the ligand-bound SCF.

FIG. 26 provides a stacked bar graph showing percent target, bystander, and non-synonymous bystander A>G edits corresponding to the indicated base editors. In FIG. 26 each bar indicates from top-to-bottom “other non-synonymous bystanders”, “1G bystander”, and “favorable” edits. In FIG. 26 “XVIVO” refers to the serum free stem cell medium in which cells were grown, “IVD” refers to “in vitro differentiated erythroid cultures (IVD)”, and “d5” and “d7” refer to five days and seven days, respectively.

FIG. 27 provides a stacked bar graph showing editing efficiencies (A to G %) for the base editors ABE8.20-NRCH (1570), ABE9v1-NRCH (2517), and ABE9v2 (2518) used in combination with the guide gRNA931 (CC200). In each stacked bar of FIG. 27, the following edits are shown in order from top-to-bottom: “3G_4G_6G,” “4G_6G,” “3G_6G,” and “6G.”

FIG. 28 provides histograms showing that cells expressing CD117 polypeptides altered using ABE8.20-NRCH (1570), ABE9v1-NRCH (2517), or ABE9v2 (2518) in combination with the guide gRNA931 (CC200) showed reduced binding to the antibody ABTx052. Cells were evaluated 2 days post-electroporation (EP). In FIG. 28, “1” indicates cells edited using ABE8.20-NRCH, “2” indicates cells edited using ABE9v1-NRCH, and “3” indicates cells edited using ABE9v2.

FIGS. 29A-29C provide bar graphs, a flow cytometry histogram, and plots showing that highly efficient multiplex editing of CD117 and HBG1/2 was achieved in CD34+ hematopoietic stem cells (HSPCs). FIG. 29A provides bar graphs showing successful multiplex base-editing of CD34+ cells using sgRNAs against polynucleotides encoding CD117 and HBG1/2. Multiplex base editing efficiency was equivalent to single-plex for both sgRNAs used. Multiplex editing of HBG1/2 and CD117 polynucleotides led to efficient editing of both with efficiencies of greater than 85%. Single clonal analysis revealed that all cells had HBG1/2 editing in the multiplex editing condition. CD117 multiplex editing outcomes were: >97% bi-allelic, 1.5% mono-allelic, and 1.5% unedited. FIG. 29B provides plots showing that multiplex base editing of a CD117 polynucleotide in combination with editing of an HBG1/2 polynucleotide did not hinder γ-globin induction in edited cells. About 60% gamma globin induction was detected in IVED cells differentiated from multiplex edited CD34+ cells. FIG. 29C provides a flow cytometry histogram showing that multiplex edited cells escaped recognition by mAb-7.

FIG. 30 provides a bar graph showing SCF blocking by mAb-7 significantly inhibited erythroid colony formation. Contacting cells with mAb-7 (i.e., ABTx052) led to enrichment of CD117 base edited cells. A 1:1 mixture of unedited and cells base-edited using the guide CC128 in combination with a base editor were plated in semi-solid media in the presence of varying concentrations of mAb-7. At day-14 individual erythroid colonies (BFU-E) were picked and sequenced (n=24 colonies were sequenced for each condition). In the absence of mAB-7, an even distribution of edited and unedited colonies was observed. Increasing concentration of mAB-7 yielded higher proportions of edited colonies. 100% of colonies at mAB-7 concentrations of 100 ng/ml or higher contained either the 5G or 5G_7G edit. Very few BFU-E colonies were observed upon incubation with SR1. A few of the selected colonies at higher antibody concentrations contained CD117 edit in only one allele indicating that a monoallelic editing may be sufficient to evade antibody blocking by mAb.

FIGS. 31A and 31B provide a bar graph and stacked histograms showing successful multiplex editing of both a CD117 polynucleotide and a beta globin gene in CD34+ hematopoietic stem cells. FIG. 31A provides a bar graph showing high multiplex editing at the sites targeted by the guides sgRNA_017 (Makassar sgRNA) and gRNA931 (CD117 sgRNA; CC200). Highly efficient CD117 and Makassar editing (n=3) was achieved in mobilized peripheral blood-derived (mPB) CD34+ cells using a single base editor in combination with two guide polynucleotides (sgRNAs). FIG. 31B provides a flow cytometry histogram showing that both edited and unedited cells were bound by a pan CD117 antibody (Pan CD117 Ab), but that the edited cells showed reduced binding to the antibody mAb-7 (i.e., ABTx052).

FIGS. 32A-32C provide structural images and a plot. The images of FIGS. 32A and 32B correspond to a ˜3.0 Å Cryo-EM structural images of CD117/c-KIT in complex with ABTx052-Fab in solution state. In FIG. 32A, each domain is numbered and each number are placed next to the corresponding domain in the figure, where “1” represents the ABTx052 Fab-heavy chain domain, “2” represents the ABTx052 Fab-light chain, “3” represents the D3 domain of CD117, “4” represents the D2 domain of CD117, and “5” represents the D1 domain of CD117. FIG. 32B provides a structural image shaded to indicate structural resolution as estimated by windowed FSC in RELION 3. FIG. 32C provides a plot of Fourier shell correlation against resolution (1/Å) showing that the final resolution of the structure was about 3 Å.

FIGS. 33A-33D provide a bar graph and plots demonstrating that alteration of the Fc-domain of mAb-7 generated highly potent mAb that did not produce mast cell degranulation in vitro. FIG. 33A provides a bar graph showing that Fc engineered versions of anti-CD117 ABTx052 did not produce mast cell degranulation in vitro. Mast cells generated via in vitro differentiation of CD34+ cells contacted with IgE or interferon gamma to increase expression of the Fc-gamma receptor, were treated with native and Fc-engineered mAb-7. Absorbance at 405 nM measured beta hexosaminidase release and thereby represented mast cell degranulation. The positive control antibodies 104D2 and NEG085 known to cause mast cell degranulation upon incubation with mast cells were used as positive controls for degranulation. In FIG. 33A, mAb-7-FcEng-1, mAB-7-FcEng-2, and mAb-7-FcEng-3, refer to ABTx052 with the Fc modifications LALAPG, LSLTRG, and LALADA respectively. FIG. 33B provides a plot demonstrating that mAb-7 selectively depleted unedited CD34+ cells in vitro, while CD117-edited cells contacted with the antibody retained viability. FIGS. 33C and 33D provide plots showing that Fc-engineered mAb-7 (ABTx135; mAb-7 with an LALADA Fc domain alteration) elicited similar levels of cell depletion in human and Cynomolgus CD34+ cells. In FIGS. 33C and 33D CD34+ cells from human and Cynomolgus sources were cultured in presence of 100 ng/mL SCF and varying concentrations of ABTx135 for 7 days. As control, some cells were cultured without any SCF. Cell viability was assessed at day 7 using Cell titer glo reagent following manufacturer's instructions. ABTx135 elicited similar levels of cell depletion in human and Cynomolgus CD34+ cells. Cell titer glo measurements at day 7 showed that mAb-7 treatment depleted unedited cells to the level of complete SCF starvation, while CD117-edited cells retained viability.

FIGS. 34A and 34B provide schematic diagrams showing how adenine base editors (ABEs) can be used to chemically modify target bases in a polynucleotide sequence. FIG. 34A provides a schematic showing that, in embodiments, an ABE is a fusion protein comprising an evolved TadA* deaminase connected to a CRISPR-Cas enzyme. The base editor binds to a target sequence that is complementary to the guide-RNA and exposes a stretch of single-stranded DNA. The deaminase converts the target adenine into inosine (which is read as guanine by DNA polymerases) and the Cas enzyme nicks the opposite strand. The nicked strand is repaired completing the conversion of an A: T to G: C base pair. FIG. 34B provides a schematic diagram showing how naturally-occurring base changes cause Hereditary Persistence of Fetal Hemoglobin (HPFH), which protects patients from SCD/B-Thal. Base editors can reproduce these changes, leading to high, consistent levels of fetal hemoglobin. Higher fetal hemoglobin can be associated with further reductions in symptoms of a hemoglobinopathy (e.g., sickle cell disease).

FIGS. 35A and 35B provide bar graphs showing that editing of CD117 polynucleotides in CD34+ cells did not alter long-term engraftment and multi-lineage reconstitution in a rodent model. FIG. 35A provides a bar graph showing that CD117-base edited cells produced stable long-term (16-week) engraftment in NBSGW mice. Unedited (light-grey bars) and CD117-base edited (dark-grey bars) CD34+ cells produced similar levels of multi-lineage reconstitution. FIG. 35B provides a bar graph showing next-generation sequencing (NGS) results demonstrating high CD117 editing levels within different hematopoietic compartments.

FIGS. 36A to 36D provide a schematic diagram and bar graphs demonstrating that mAb-7 selectively depleted unedited cells from the bone marrow (BM) of mice transplanted with hCD34+ cells. FIG. 36A provides a schematic diagram of an in vivo study to evaluate anti-CD117-mAb-mediated myeloablation. Irradiated NSG mice were first humanized with either unedited cells or a 1:1 mixture of unedited and multiplex-edited CD34+ cells. 4 weeks post-transplantation, mice were either left untreated, or treated with either isotype control antibody or mAb-7 according to mouse groups listed in right panel. Mouse bone marrow was harvested at indicated time points and analyzed using flow cytometry. Next-generation sequencing (NGS) was performed with bulk bone marrow and sorted CD34+ cells. FIG. 36B provides bar graphs showing a significant reduction in human chimerism was observed at 8 weeks (top panel) and 12 weeks (bottom panel) post dosing. FIG. 36C provides bar graphs showing the CD34+ cell population was also abrogated at 8 (top panel) and 12-week (bottom panel) time points. FIG. 36D provides bar graphs showing editing at HBG1/2 and CD117 target sites within the bulk bone marrow (left panel) and the sorted CD34+ cells (right panel) and demonstrating significant enrichment of editing in the mAb-7 treatment group compared with the isotype control group, indicating in vivo selection of multiplex-edited cells. In FIG. 36D, each set of four bars represents, from left-to-right, the following mouse groups: D, E, D, E. In FIGS. 36A-36D the letters “A” to “E” are used to refer to the following mouse groups: A) unedited cells and no antibody treatment; B) unedited cells and treatment with a control isotype antibody; C) unedited cells and treatment with mAb-7; D) 1:1 ratio of edited and unedited cells and treatment with the control isotype antibody; E) 1:1 ratio of edited and unedited cells and treatment with mAb-7.

FIG. 37 provides a schematic diagram showing the design of an in vivo experiment to evaluate ABTx052-mediated enrichment of multiplex base-edited cells in NSG mice. The multiplex base-edited cells contained an altered CD117 polynucleotide and an altered HBG1/2 polynucleotide.

FIG. 38 provides bar graphs showing engraftment data measured by human CD45+ cell chimerism over time in mice. Depletion of unedited cells was observed following administration of mAb (ABTx052) to the mice. The mice were treated as described in FIG. 37. The study conditions referenced in FIG. 38 are described in Table 15.

FIG. 39 provides bar graphs showing HSC bone marrow analysis results demonstrating selective depletion of unedited CD34+ hematopoietic stem cells (HSCs) in mice engrafted with the cells and exposed to ABTx052 post-engraftment. The mice were treated as described in FIG. 37. The study conditions referenced in FIG. 38 are described in Table 26.1.

FIGS. 40A and 40B provide stacked bar graphs showing allelic frequencies for HBG1/2 edits and CD117 edits measured at eight weeks post ABTx052 administration in mice treated as described in FIG. 37, in bone marrow (FIG. 40A) and in CD34+ cells (FIG. 40B). The groups referenced in FIG. 38 are described in Table 15.

FIGS. 41A and 41B provide plots showing ABTx052 cell binding as measured on (FIG. 41A) M07e cells expressing wild-type (WT) or base-edited (ESCAPE-2 variant) CD117 and (FIG. 41B) mobilized peripheral blood (mPB) human CD34's expressing wild-type (WT) or base-edited (ESCAPE-2 variant) CD117. Cells expressing WT showed high affinity binding to ABTx052, while cells expressing the ESCAPE-2 variant did not. In both cell types, highly selective binding of ABTx052 was observed for WT but not for the ESCAPE-2 variant produced in the cells through base editing according to the methods provided herein.

FIGS. 42A and 42B provide a plot and a bar graph showing that ABTx052 (an SCF blocking antibody) was capable of mediating the depletion in culture of cells expressing wild-type CD117, but that cells expressing an altered CD117 polypeptide with reduced binding affinity for ABTx052 escaped depletion. ABTx052 (FIG. 42A) selectively depleted unedited hCD34+ cells in vitro and (FIG. 42B) enriched for cells base edited to express the altered CD117 polypeptide. Cells were evaluated on the fourth and tenth days following being contacted with ABTx052. The cells expressing the wild-type CD117 polypeptide were also base edited to express the Makassar variant of beta hemoglobin (HBB). In FIGS. 42A and 42B, “no SCF culture condition” indicates cell viability observed for cells grown in the absence of SCF, and “ESCAPE-2 variant” indicates cells base edited to express the altered CD117 polypeptide and the Makassar variant of HBB. In the plot of FIG. 42A, unedited, CD117-edited (ESCAPE-2 variant, CC200/gRNA931), and a 1:1 mixture of unedited and CD117-edited CD34+ cells were cultured in presence of 100 ng/mL SCF and varying concentrations of ABTx052 for 4 days prior to analysis. As a control, some cells were cultured without any SCF, and their viability is shown by the dotted line in the plot. Cell viability was assessed at day 4 following being contacted with ABTx052 using a Cell titer glo reagent following manufacturer's instructions. Treatment with ABTx052 depleted unedited cells to the level of complete SCF starvation (dotted line), while CD117-edited cells retain viability. Cell viability levels for the mixed population maintained an intermediate value. The bar graph of FIG. 42B shows that next-generation sequencing of genomic DNA isolated from untreated and ABTx052 treated cells at day 10 demonstrated enrichment of CD117 editing in the treated cells compared with the untreated control.

FIG. 43 provides a Western blot image showing that both bi and mono-allelically multiplex base edited M07e cells base edited to express an altered CD117 polypeptide with reduced binding to ABTx052 and the Makassar variant of beta hemoglobin (HBB) variant retained CD117 phosphorylation upon stimulation with SCF, even in the presence of ABTx052. Phosphorylation of WT CD117 was blocked upon being contacted with ABTx052.

FIGS. 44A and 44B provide bar graphs showing in vivo depletion of unedited hematopoietic stem and progenitor cells (HSPCs) 2 weeks post-dosing with ABTx052 or ABTx135 (4 week post-humanization). FIG. 44A provides bar graphs showing total chimerism (left bar graph) and Lin−CD34+CD117+HSPC frequencies (right bar graph) of mice (n=3) dosed with ABTx135. FIG. 44B provides a bar graph showing chimerism levels measured in the mice at later time points (8 wk and 12 wk following dosing with ABTx052). In FIGS. 44A and 44B, “mAb” represents the ABTx052 or ABTx135 antibody.

FIGS. 45A-45K provide plots showing kinetics for binding of ABTx052 and variants thereof to wild-type CD117 (Wt CD117) and to the indicated two CD117 variants (CC200 and CC128) expressed from polynucleotides prepared by base editing a polynucleotide encoding wild-type CD117 using a base editor system containing the guide polynucleotide CC200 or CC128 and an adenosine deaminase base editor (ABE). In each of FIGS. 45A-45K, the y-axis represents antibody binding to CD117 in nanomolar and the x-axis represents time. The first vertical line from the left in each of FIGS. 45A-45K represents the time at which CD117 was first contacted with a solution containing the indicated antibody, and the second vertical line from the left represents the time at which the solution in contact with CD117 was replaced with a solution free of any antibody. In FIGS. 45B-45K, the indicated ABTx052 variants each contain a single amino acid alteration, as indicated, in the indicated chain of ABTx052 (i.e., heavy chain variable region (VH) or light chain variable region (VL)). All of the antibodies evaluated were human IgG1 antibodies.

FIGS. 46A and 46B provide flow cytometry histograms demonstrating reduced binding of the indicated ABTx052 variants to the CD117 variant CC128 expressed from a polynucleotide prepared by base editing a polynucleotide encoding wild-type CD117 using a base editor system containing the guide polynucleotide CC128 and an adenosine deaminase base editor (ABE). In FIGS. 46A and 46B, the indicated ABTx052 variants, with the exception of ABTx062, each contain a single amino acid alteration, as indicated, in the indicated chain of ABTx052 (i.e., heavy chain variable region (VH) or light chain variable region (VL)). All of the antibodies evaluated were human IgG1 antibodies.

FIG. 47 provides a schematic diagram summarizing some representative advantages of the methods provided herein compared to conditioning methods involving administering an alkylating agent (e.g., busulfan) or an anti-CD117 monoclonal antibody to a subject.

FIG. 48 provides a plot showing biolayer interferometry (BLI) measurements demonstrating comparable binding of wild-type and CC128-edited CD117 polypeptides to SCF.

FIG. 49 provides a plot showing wild-type and CC128-edited CD117 showed similar internalization upon SCF stimulation as measured by surface availability of CD117.

FIGS. 50A and 50B present flow cytometry contour plots and a stacked bar graph showing that hematopoietic stem and progenitor cells (HSPCs) base edited using a base editor system containing the guide CC128 and the base editor ABE8.8 retained normal myeloid and erythroid differentiation in vitro. FIG. 50A provides flow cytometry contour plots showing different myeloid cells generated via in vitro differentiation of the edited and unedited cells. Unedited and edited cells (CC128 cells) showed equivalent myeloid differentiation potential to generate neutrophils, monocytes, eosinophils, basophils, dendritic cells, and plasmocytoid dendritic cells (pDCs) in vitro. FIG. 50B provides a stacked bar graph showing results from colony forming unit (CFU) assays demonstrating that the edited HSPCs had similar colony forming efficiency and comparable colony output to unedited cells. In FIG. 50A, the numbers next to each box represent the percent of total cells counted that fell within the region delimited by the box.

FIGS. 51A and 51B provide flow cytometry contour plots and a plot showing that hematopoietic stem and progenitor cells (HSPCs) were sensitive to treatment with mAb-7. FIG. 51A provides flow cytometry contour plots showing a gating and sorting strategy for analysis of multipotent long-term hematopoietic stem cell (LT-HSC), multipotent progenitor cell (MPP), common myeloid progenitor cell (CMP), granulocyte-monocyte progenitor cell (GMP), and megakaryocyte-erythroid progenitor cell (MEP) subpopulations of cells contacted with mAb-7 based on cell surface marker expression. Healthy human mobilized peripheral blood derived CD34+HSPCs were stained with cell surface markers to identify long-term HSC, multi-potent progenitor, common myeloid progenitor, granulo-monocytic progenitor, and megakaryocytic-Erythrocytic progenitor subpopulations. These populations were isolated using flow assisted sorting to perform antibody treatment experiments. FIG. 51B provides a plot showing that LT-HSC and other progenitor subpopulations within CD34+ fractions exhibited similar sensitivity to mAb-7 treatment in vitro. Sorted HSPC subpopulations were seeded in a 96-well plate and treated with varying concentrations of ABTx135 and cultured for 5 days in 37° C. tissue culture incubator. Cell viability was measured at day 5 using cell titer glo assay.

FIGS. 52A-52C provide flow cytometry histograms, a flow cytometry contour plot, and a plot showing that anti-CD117 mAb treatment using mAb-7 induced HSPC apoptosis in vitro. FIG. 52A provides flow cytometry histograms showing that mAb-7 treatment mimicked SCF withdrawal and led to proliferation arrest in CD34+HSPCs as measured by cell-trace violet dye dilution. Cell-trace violet dye was used to measure cell proliferation. FIG. 52B provides flow cytometry contour plots showing a gating strategy to identify live, early apoptotic, and late apoptotic cells based on Annexin V and 7-AAD staining. FIG. 52C provides a plot of aggregated data showing frequency of live, apoptotic, and necrotic cells and demonstrating that mAb-7 treatment led to dose-dependent apoptosis in unedited HSPCs.

FIGS. 53A-53E provide a schematic diagram and bar graph showing engraftment of HSPCs base edited using the guide CC128 and the base editor ABE8.8 following anti-CD117 mAb-based conditioning. FIG. 53A provides a schematic diagram showing the design of the in vivo experiment to which the data of FIGS. 53B-53E corresponds. FIG. 53B provides bar graphs showing enrichment of CD117 (CC128) and HBG1/2 base editing in group 2 mice, which indicated engraftment of multiplex edited cells in mice treated with mAb-7. The isotype control group (group 1) showed absence of marrow editing. In FIG. 53B, each grouping of 5 bars represent, from left-to-right, bulk BM, hCD15, hCD19, Lin−CD34+, and GlyA. FIG. 53C provides bar graphs showing high human chimerism in an mAb-7 treated group that received a transplant of multiplex edited cells (group 2), in bulk bone marrow (Top panel) and Lin−CD34+ (Bottom panel) compartments. FIG. 53D provides a bar graph showing depletion of HPSCs expressing WT-CD117 and enrichment of base edited HSPCs (CC128) in bone marrow. FIG. 53E provides a bar graph presenting results from a receptor occupancy assay showing occupied receptors on unedited human HSPCs in mouse bone marrow following mAb treatment. The transplant dosing groups 1 and 2 of FIGS. 53B-53E are described in Table 19.

FIG. 54 provides plots showing kinetics for binding of ABTx052 and the VH_E107D (ABTx181) variant of ABTx052 to wild-type CD117.

FIGS. 55A-55C provide plots showing kinetics for binding of ABTx052 and each of the antibodies listed in Table 21 to wild-type CD117 (FIG. 55A), to the CC128 variant of CD117 (FIG. 55B), and to the CC200 variant of CD117 (FIG. 55C). The CC200 and CC128 variants of CD117 were prepared by base editing cells using the guide CC200 or CC128, respectively, and an adenosine deaminase base editor (ABE). In each of FIGS. 55A-55C, the y-axis represents antibody binding to CD117 in nanomolar, and the x-axis represents time. The first vertical line from the left in each of FIGS. 55A-55C represents the time at which CD117 was first contacted with a solution containing the indicated antibody, and the second vertical line from the left represents the time at which the solution in contact with CD117 was replaced with a solution free of any antibody.

FIG. 56 provides a plot similar to those of FIGS. 55A-55C showing kinetics for binding of ABTx052 to wild-type CD117, to the CC128 variant of CD117, and to the CC200 variant of CD117.

FIGS. 57A-57L provide plots similar to those of FIGS. 55A-55C showing kinetics for binding of ABTx248 (FIG. 57A), ABTx253 (FIG. 57B), ABTx271 (FIG. 57C), ABTx273 (FIG. 57D), ABTx250 (FIG. 57E), ABTx251 (FIG. 57F), ABTx274 (FIG. 57G), ABTx265 (FIG. 57H), ABTx268 (FIG. 57I), ABTx270 (FIG. 57J), ABTx256 (FIG. 57K), and ABTx272 (FIG. 57L) to wild-type CD117, to the CC128 variant of CD117, and to the CC200 variant of CD117.

FIGS. 58A-58D provide plots similar to those of FIGS. 55A-55C showing kinetics for binding of ABTx052 (FIG. 58A), ABTx248 (FIG. 58B), ABTx253 (FIG. 58C), and ABTx271 (FIG. 58D), to wild type CD117 at different antibody concentrations, where each curve of each plot represents a different antibody concentration. The following antibody concentrations were evaluted: 333.3 nM, 166.7 nM, 83.3 nM, 41.6 nM, 20.8 nM, 10.4 nM, and 5.2 nM.

FIG. 59 provides a plot showing EC50 values for the indicated antibodies when administered to M07e cells. In FIG. 59, “MFI” indicates “mean fluorescence intensity.”

FIG. 60 provides melt curves for the antibodies ABTx248, ABTx253, ABTx271, and ABTx052 showing that the ABTx247, ABTx253, and ABTx271 antibodies had Tms that were comparable to those of ABTx052.

FIG. 61 provides a plot showing that the antibodies ABTx248, ABTx253, and ABTx271 has low polyspecificity.

FIG. 62 provides a plot showing that hematopoietic stem cells (HSCs) contacted with increasing concentrations of the antibody ABTx052, ABTx167, ABTx248, ABTx135, ABTx253, or ABTx271 showed reductions in viability. Unedited HSCs were treated with the indicated monoclonal antibodies for 5-7 days. The media used was StemSpan™ SFEM II+Glutamax+100 ng/mL+Pen/Strep with 100 ng/mL SCF. Cells cultured without SCF was used as a reference.

FIG. 63 provides a schematic diagram showing two dosing paradigms used for transplantation of CD117 edited hematopoietic stem cells (HSCs) into humanized NBSGW mice following administration of an anti-CD117 monoclonal antibody (ABTx135). The groups of mice treated according to Dosing Paradigm A or Dosing Paradigm B are described in Tables 26 and 27, respectively. In FIG. 63, “CD117 edited” indicates that the cells were edited using the base editor ABE8.8 in combination with sgRNA CC128 (gRNA434).

FIGS. 64A-64G provide a series of bar graphs. FIG. 64A provides a bar graph showing chimerism observed in mice treated according to Dosing Paradigm A (see FIG. 63) at 4 weeks following administration of 1e6 CD34+ cells from Donor A and prior to administration of the monoclonal antibody ABTx135. FIGS. 64B-64G providing bar graphs showing chimerism (FIG. 64B) as well as frequencies of the cell types Lin−CD34+CD117+ (FIG. 64C), Lin−CD34+ (FIG. 64D), CD34+ (FIG. 64E), CD15+ (FIG. 64F), and GlyA+ (FIG. 64G) at 2 weeks post-monoclonal antibody administration. As a control, mice were administered an isotype antibody that does not bind CD117.

FIGS. 65A and 65B provide bar graphs showing observed levels of target editing of a CD117 polynucleotide (FIG. 65A) and/or an HBG1/2 promoter region (FIG. 65B) in bulk bone marrow (BM), hCD15+ cells, hCD19+ cells, Lin−CD34+ cells, and GlyA+ cells in mice treated according to Dosing Paradigm B of FIG. 63 at 10 weeks post monoclonal antibody administration. In FIGS. 65A and 65B, each grouping of five bars corresponds, from left-to-right, to “Bulk BM,” “hCD15,” “hCD19,” “Lin−CD34+,” and “GlyA.” The four groups indicated along the x-axis of FIGS. 65A and 65B correspond, respectively, to Groups 1˜4 of Table 27.

FIGS. 66A-66C provide bar graphs showing levels of chimerism (FIG. 66A), Lin−CD34+ cells (FIG. 66B), and CD34+ cells (FIG. 66C) in mice treated according to Dosing Paradigm B of FIG. 63 at 10 weeks post monoclonal antibody administration. In FIGS. 66A-66C, Groups 1B, 2B, 3B, and 4B, correspond to Groups 1, 2, 3, and 4 of Table 27, respectively.

FIGS. 67A-67D provide bar graphs showing levels of CD19+ cells (FIG. 67A), CD15+ cells (FIG. 67B), GlyA+ cells (FIG. 67C), and CD33+ cells (FIG. 67D) in mice treated according to Dosing Paradigm B of FIG. 63 at 10 weeks post monoclonal antibody administration. In FIGS. 67A-67C, Groups 1B, 2B, 3B, and 4B, correspond to Groups 1, 2, 3, and 4 of Table 27, respectively.

FIG. 68 provides a bar graph showing levels of Lin−CD34+CD117+ cells expressing wild type CD117 or edited CD117 in mice treated according to Dosing Paradigm B of FIG. 63 at weeks post monoclonal antibody administration. In FIG. 68 Groups 1, 2, 3, and 4 correspond to Groups 1, 2, 3, and 4 of Table 27, respectively.

FIG. 69 provides a schematic diagram showing two dosing paradigms used for transplantation of CD117 edited hematopoietic stem cells (HSCs) into NSG-SGM3 mice expressing human stem cell factor (SCF). The groups of mice treated according to the protocol described in FIG. 69 are described in Table 69.

FIGS. 70A-70C provide bar graphs showing levels of target base editing observed in the bulk bone marrow of mice treated according to the method of FIG. 69 at 4 weeks post hCD34+ cell transplantation. FIG. 70A provides a bar graph showing levels of editing observed at a CD117 polynucleotide target site corresponding to the guide polynucleotide CC128. FIG. 70B provides a bar graph showing levels of editing observed at a CD117 polynucleotide target site corresponding to the guide polynucleotide CC200. FIG. 70C provides a bar graphs showing levels of editing observed at a HBG1/2 promoter target site corresponding to the guide CC200. The groups indicated along the x-axis of FIGS. 70A-70C correspond, respectively, to Groups 1˜4 of Table 69.

FIGS. 71A-71F provide bar graphs showing levels of chimerism (FIG. 71A), Lin−CD34+ cells (FIG. 71B), CD34+ cells (FIG. 71C), CD19+ cells (FIG. 71D), CD15+ cells (FIG. 71E), and CD33+ cells (FIG. 71F) in mice treated according to the method of FIG. 69 at 4 weeks post hCD34+ cell transplant. The groups indicated along the x-axis of FIGS. 71A-71F correspond, respectively, to Groups 1˜4 of Table 69.

FIGS. 72A and 72B provide plots showing kinetics for binding of variants of ABTx025 to wild-type CD117 (Wt CD117) (FIG. 72A) and to the CD117 variant CC128 (FIG. 72B) expressed from a polynucleotide prepared by base editing a polynucleotide encoding wild-type CD117 using a base editor system containing the guide polynucleotide CC128 and an adenosine deaminase base editor (ABE). In each of FIGS. 72A and 72B, the y-axis represents antibody binding in nanomolar and the x-axis represents time. The first vertical line from the left in each of FIGS. 72A and 72B represents the time at which CD117 was first contacted with a solution containing an antibody, and the second vertical line from the left represents the time at which the solution in contact with CD117 was replaced with a solution free of any antibody.

FIGS. 73A-73E provide plots showing kinetics for binding of variants of ABTx025 to wild-type CD117 (Wt CD117) (FIG. 72A). In each of FIGS. 73A-73E, the y-axis represents antibody binding in nanomolar and the x-axis represents time. In each of FIGS. 73A-73E, the vertical line represents the time at which the solution in contact with CD117 was replaced with a solution free of any antibody. The first vertical line from the left in each of FIGS. 73A-73E represents the time at which CD117 was first contacted with a solution containing the indicated antibody, and the second vertical line from the left represents the time at which the solution in contact with CD117 was replaced with a solution free of any antibody. The plots of FIGS. 73A-73E show the off rates for the indicated antibodies. Each line of the plots of FIGS. 73A-73E corresponds to a different concentration of CD117.

FIGS. 74A and 74B provide melt curves for the indicated antibodies. FIG. 74B plots the derivative of the melt curves shown in FIG. 74A.

FIG. 75 provides a plot showing that the indicated antibodies had low polyspecificity.

FIGS. 76A and 76B show binding of the indicated antibodies to M07e cells expressing wild type CD117 (FIG. 76A) or a CC128 variant of CD117 (FIG. 76B). The plots allow for the determination of an effective concentration 50% (EC50) value for the binding of each antibody to the indicated cells. The cells were stained using the indicated antibodies as the primary antibody and a secondary antibody containing the fluorophore AF647.

FIG. 77 provides a plot showing that the antibodies ABTx196 and ABTx198 were able to efficiently deplete human CD34+HSPCs at efficiencies comparable to those for the antibody ABTx135. The horizontal line in FIG. 77 corresponds to the viability of cells grown in the absence of SCF.

FIGS. 78A-78D providing size exclusion chromatography-high performance liquid chromatography (SEC-HPLC) plots showing the purity of ABTx307, ABTx308, ABTx309, and ABTx313 antibodies isolated from host cells.

FIG. 79 provides images of reducing and non-reducing SDS PAGE gels run to evaluate purified ABTx307, ABTx308, ABTx309, and ABTx313 antibodies.

FIGS. 80A and 80B provide melt curves for the indicated antibodies. FIG. 80B plots the derivative of the melt curves shown in FIG. 80A.

FIGS. 81A and 81B provide plots showing polyspecificity levels for each of the indicated antibodies as evaluated using a 1:200 baculovirus particles (BVP) coating (FIG. 81A) and a 1:500 BVP coating (FIG. 81B).

FIGS. 82A-82E provide plots showing KD measurements for ABTx307 (FIG. 82A), ABTx308 (FIG. 82B), ABTx309 (FIG. 82C), ABTx313 (FIG. 82D), and ABTx135 (FIG. 82E). KD measurements were collected using Anti-Human IgG Fc Capture (AHC) biosensors to capture 100 nM of each monoclonal antibody followed by incubation with a 1:2 titration series of a wild type CD117 extracellular domain (ECD) protein with an undiluted concentration of 345 nM. The AHC biosensor data was analyzed using Octet Analysis Studio software.

FIGS. 83A-83E provide plots showing binding to the CC128 variant of CD117 for ABTx307 (FIG. 83A), ABTx308 (FIG. 83B), ABTx309 (FIG. 83C), ABTx313 (FIG. 83D), and ABTx135 (FIG. 83E). Binding to the CC128 variant of CD117 was determined using streptavidin (SA) biosensors, which were allowed to bind the CC128 variant from a 5 μg/ml solution thereof. The SA biosensors were then titrated with monoclonal antibodies starting at 2.5 μM and using titration increments of 1:2. Data was analyzed using the Octet Analysis Studio Software.

FIG. 84 provides plots showing binding to wild type CD117 and the CC128 variant of CD117 for ABTx135, ABTx309, ABTx307, ABTx313, and ABTx308. Binding to the CC128 variant of CD117 or to wild type CD117 was determined using streptavidin (SA) biosensors, which were allowed to bind the CD117 polypeptide from a 5 μg/ml solution thereof. The SA biosensors were then incubated with 100 nM of the indicated antibodies. Data was analyzed using the Octet Analysis Studio Software.

FIG. 85 provides a plot showing binding of ABTx308 and ABTx253 to the CC128 variant of CD117. Binding to the CC128 variant of CD117 was determined using streptavidin (SA) biosensors, which were allowed bind the CC128 variant from a 5 μg/ml solution thereof. The SA biosensors were then incubated with 100 nM of the indicated antibodies. Data was analyzed using the Octet Analysis Studio Software.

FIG. 86A-86D provide plot comparing binding to the CC128 variant of CD117 by ABTx307 (FIG. 86A), ABTx308 (FIG. 86B), ABTx309 (FIG. 86C), and ABTx313 (FIG. 86D) to ABTx135. Binding to the CC128 variant of CD117 was determined using streptavidin (SA) biosensors, which were allowed to bind the CC128 variant from a 5 μg/ml solution thereof. The SA biosensors were then incubated with 100 nM of the indicated antibodies. Data was analyzed using the Octet Analysis Studio Software.

FIGS. 87A and 87B provide plots showing binding of M07e cells expressing wild type CD117 (FIG. 87A) and of M07e cells expressing a CC128 variant of CD117 (FIG. 87B) by ABTx307, ABTx308, ABTx308, ABTx313, and ABTx135. The indicated monoclonal antibodies were incubated with the cells for 1 hour. The chart of FIG. 87A provides a summer of the EC50 (nM) values calculated for each antibody from the plot of FIG. 87A.

FIG. 88 provides a plot showing that the antibodies ABTx307, ABTx308, ABTx309, and ABTx313 were able to efficiently deplete human CD34+HSPCs. The horizontal line in FIG. 77 corresponds to the viability of cells grown in the absence of SCF.

FIG. 89 provides a ribbon diagram showing a cryo-EM structure of a CD117: ABTx052 complex. The CD117 domains D1, D2, and D3 are shown in different shades of grey, and the antibody ABTx052 is shown in darker shades of grey to the right of the CD117 domains in FIG. 89. The CD117 domains D4 and D5 were excluded from the structure due to the lack of an interpretable cryo-EM density map. The CD117 residues [D72 and T74 (domain D1); D121, R122, Y125, R181, and K203 (domain D2); and S240, E257, Y259, S261, and H263 (domain D3)] that interact with ABTx052 are shown as spheres.

FIG. 90 provides a ribbon diagram showing a cryo-EM structure of a CD117: SCF complex. The superposition between CC128: SCF (CC128-arrows/beta pleated sheets shown in lighter shades of grey, SCF-spirals/alpha helices shown in darker shades of grey; CC128 domains D5 and D5′ were excluded from the structure due to the lack of interpretable cryo-EM density map) and CD117: SCF (CD117, SCF; PDB Accession No. 2E9W) tetrameric structures revealed high structural similarity, with an RMSD of 0.848-Å for all of the Ca atoms. This high structural similarity between these complexes of CD117 and CC128 with SCF showed that the CC128-Y259C—N260D substitutions did not affect the protein structure and, consequently, its function in binding SCF.

### DETAILED DESCRIPTION

The disclosure features compositions and methods for non-genotoxic monoclonal antibody (mAb) conditioning, where the methods involve altering a cluster of differentiation 117 (CD117; c-KIT) polynucleotide sequence in a hematopoietic stem cell (HSC) or progenitor thereof to encode a CD117 polypeptide with reduced binding to the antibody. In various embodiments, the methods further include introducing a therapeutic alteration to a gene of the HSC or progenitor thereof for treatment of a hemoglobinopathy (e.g., sickle cell disease).

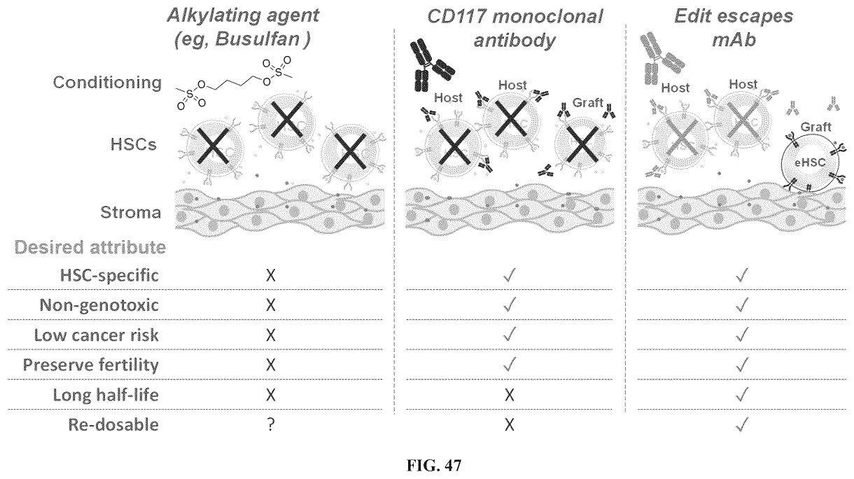

The embodiments of the disclosure are based, at least in part, upon the development of a non-genotoxic, busulfan-free, conditioning approach that combines a non-genotoxic conditioning strategy with a gene editing strategy for the treatment of sickle cell disease. The embodiments of the disclosure were developed with the goal, among others, of reducing challenges associated with the standard of care conditioning regimen (see, e.g., FIG. 47). The non-genotoxic conditioning efforts aimed to leverage base editing technologies to engineer hemopoietic stem cells (eHSCs) that can be coupled with a monoclonal antibody (mAb) reagent capable of conditioning a patient prior to transplantation. Through base editing, an engineered stem cell antibody paired evasion, or “ESCAPE”, approach was developed that enables the engineered HSCs to be resistant to mAb still present in the system and/or allows the ability to administer a second dose of the mAb after administration of the HSCs. In embodiments, the engineered hematopoietic stem cells (eHSCs) can selectively escape ablation and be engineered to contain base edits in the promoter region of the HBG1/2 gene and/or to contain a Makassar alteration to a pathogenic beta globin gene, towards a potential treatment of sickle cell disease.

The methods described herein provide for the selective targeting of endogenous HSCs, while sparing edited HSCs. Accordingly, antibody or antibody-drug conjugate (ADC) treatment used for conditioning prior to hematopoietic stem cell transplantation (HSCT) can continue to be administered following HSCT to expand gene edited cells in vivo or treat malignant disease with repeated dosing. This minimizes the risk of killing edited cells. Edited cells would allow for the administration of antibody or ADC without Fc modifications to reduce their half-life. This has the potential to enable the use of antibodies with longer half-lives and simplify the development of ADCs. Clinical trial design is also simplified-HSCs could be infused prior to or concurrently with conditioning with little or no risk of being depleted. The methods provide a benefit for all patients regardless of immune status.

In one embodiment, CD117 is altered (e.g., using base editing) in a cell for transplantation to prevent binding of anti-CD117 antibody, but not interfere with normal SCF signaling. Using base editing, a nucleobase change may be generated to create an amino acid substitution in CD117. Stem cell factor (SCF) drives HSC self-renewal and differentiation into progenitor cells. Administering anti-CD117 antibody blocks SCF binding to CD117, thereby depleting HSCs and progenitor cells in the patient (conditioning). Autologous gene edited HSCs are transplanted into the patient. Gene-edited cells compete with residual host HSCs to repopulate bone marrow (BM). Anti-CD117 antibody blocks SCF binding to wildtype (WT) CD117 but cannot bind to HSCs with an edited CD117. Thus, native, wild type HSCs are targeted by anti-CD117 antibody, but gene edited HSCs are not. Thus, both cells express CD117 polypeptides that are activated by SCF binding. However, binding of anti-CD117 antibody to wild type CD117 disrupts SCF binding and results in inhibition of SCF signaling wild type cells. In contrast, gene-edited HSCs are refractory to anti-CD117 antibody because the amino acid substitution introduced in CD117 prevents the binding of anti-CD117 antibody but does not interfere with normal SCF binding and signaling.

In embodiments, the methods of the disclosure enable a reduction or elimination of use of alkylating agents for conditioning. In some instances, the reduction is a reduction of about, or at least about, 5%, 10%, 15%, 20%, 25%, 30%, 35%, 40%, 45%, 50%, 55%, 60%, 65%, 70%, 75%, 80%, 85%, 90%, 95%, or 100%. In some instances, the methods of the disclosure are associated with a reduction in or avoidance of side effects associated with an alkylating agent (e.g., busulphan). In some cases, the side effect is selected from one or more of intestinal mucosal damage, alopecia, pancytopenia, anemia, amenorrhea, impaired spermatogenesis, increased risk of malignancy, and infertility.

Exemplary, yet nonlimiting, examples of compositions and methods for treating hemoglobinopathies are described in International Publications No. WO2021041945, WO2020168133, WO 2019/217942, and WO2019079347, which are incorporated herein by reference in their entireties for all purposes.

### Cluster of Differentiation 117 (Cd117; C-Kit)

CD117 is expressed in hematopoietic stem cells (HSCs) and is critical for their self-renewal, survival & differentiation. Upon differentiation, CD117 expression is lost. Mature mast cells retain CD117 expression. High level of expression the long term and short-term HSCs make CD117 an attractive target for immunologic conditioning.

Therefore, one approach to eliminate hematopoietic stem cells from a niche is to contact the cells with an anti-CD117 antibody that interferes with proper functioning of the CD117 polypeptide (e.g., blocks binding to SCF). Accordingly, CD117/c-KIT is a target for hematopoietic stem cell transplantation (HSCT) antibody-based conditioning. Non-limiting examples of antibodies suitable for use in the methods of the disclosure include ABTx052, ABTx062, ABTx025, ABTx030, ABTx070, ABTx071, JSP191, and MGTA-117. In some embodiments, the antibody is selected from one or more of ABTx025, ABTx030, ABTx052, ABTx061, ABTx062, ABTx070, ABTx071, ABTx196, ABTx198, ABTx202, ABTx203, ABTx205, ABTx206, ABTx248, ABTx250, ABTx251, ABTx253, ABTx254, ABTx255, ABTx256, ABTx265, ABTx268, ABTx270, ABTx271, ABTx272, ABTx273, ABTx274, ABTx307, ABTx308, ABTx309, and ABTx313.

Critical events in the DCD117 life cycle include SCF binding, CD117 homo-dimerization, trans-phosphorylation of tyrosine residues, ubiquitinization, internalization, and proteolytic degradation. Trans-phosphorylation of the tyrosine residues is associated with cell activation and downstream phosphorylation, calcium mobilization, and cell migration.

### HBB Gene Editing

As described herein, the compositions and methods of the disclosure are useful and advantageous for the treatment of sickle cell disease (SCD), which is caused by a Glu→Val mutation at the sixth amino acid of the β-globin protein encoded by the HBB gene. Despite many developments to date in the field of gene editing, precise correction of the diseased HBB gene to revert Val→Glu is presently not achievable using either CRISPR/Cas nuclease or CRISPR/Cas base editing approaches.

Genome editing of the HBB gene to replace the affected nucleotide using a CRISPR/Cas nuclease approach requires cleavage of genomic DNA. However, cleavage of genomic DNA carries an increased risk of generating base insertions/deletions (indels), which have the potential to cause unintended and undesirable consequences, including generating premature stop codons, altering the codon reading frame, etc. Furthermore, generating double-stranded breaks at the β-globin locus has the potential to radically alter the locus through recombination events. The β-globin locus contains a cluster of globin genes having sequence identity to one another -5′-ε-; Gγ-; Aγ-; δ-; and β-globin-3′. Because of the structure of the β-globin locus, recombination repair of a double-stranded break within the locus has the potential to result in gene loss of intervening sequences between globin genes, for example between δ- and β-globin genes.

Unintended alterations to the locus also carry a risk of causing thalassemia.

CRISPR/Cas base editing approaches hold promise in that they have the ability to generate precise alterations at the nucleobase level. However, precise correction of Val→Glu (GTG→GAG) requires a T·A to A·T transversion editor, which is not presently known to exist. Additionally, the specificity of CRISPR/Cas base editing is due in part to a limited window of editable nucleotides created by R-loop formation upon CRISPR/Cas binding to DNA. Thus, CRISPR/Cas targeting must occur at or near the sickle cell site to allow base editing to be possible, and there may be additional sequence requirements for optimal editing within the window. One requirement for CRISPR/Cas targeting is the presence of a protospacer-adjacent motif (PAM) flanking the site to be targeted. For example, many base editors are based on SpCas9 which requires an NGG PAM. Even assuming hypothetically that an T·A to A·T transversion were possible, no NGG PAM exists that would place the target “A” at a desirable position for such an SpCas9 base editor. Although many new CRISPR/Cas proteins have been discovered or generated that expand the collection of available PAMs, PAM requirements remain a limiting factor in the ability to direct CRISPR/Cas base editors to specific nucleotides at any location in the genome.

The present disclosure is based, at least in part, on several discoveries described herein that address the foregoing challenges for providing a genome editing approach for treatment of sickle cell anemia. In one aspect, the embodiments of the disclosure are based in part on the ability to replace the valine at amino acid position 6, which causes sickle cell disease, with an alanine, to thereby generate an Hb variant (Hb Makassar) that does not generate a sickle cell phenotype. While precise correction (GTG→GAG) is not possible without a T·A to A·T transversion base editor, the studies performed herein have found that a Val→Ala (GTG→GCG) replacement (i.e., the Hb Makassar variant) can be generated using an A·T to G·C base editor (ABE). In some embodiments, the methods of the disclosure involve detecting an Hb Makassar variant using an antibody, such as an antibody selected from those described in U.S. Provisional Patent Application No. 63/329,109, filed Apr. 8, 2022. This was achieved in part by the development of novel base editors and novel base editing strategies, as provided herein. For example, novel ABE base editors (i.e., having an adenosine deaminase domain) that utilize flanking sequences (e.g., PAM sequences; zinc finger binding sequences) for optimal base editing at the sickle cell target site.

Thus, the present disclosure includes compositions and methods for base editing a thymidine (T) to a cytidine (C) in the codon of the sixth amino acid of a sickle cell disease variant of the β-globin protein (Sickle HbS; E6V), thereby substituting an alanine for a valine (V6A) at this amino acid position. Substitution of alanine for valine at position 6 of HbS generates a β-globin protein variant that does not have a sickle cell phenotype (e.g., does not have the potential to polymerize as in the case of the pathogenic variant HbS). Accordingly, the compositions and methods of the disclosure are useful for the treatment of sickle cell disease (SCD).

HBG1 and/or HBG2 Promoter Editing

Sickle cell disease (SCD) affects approximately 100,000 patients in the United States. Individuals carrying both the SCD mutation and mutations that cause persistence of fetal hemoglobin (HPFH) do not typically present with sickle cell pathologies due to persistent fetal hemoglobin (HbF) levels. Higher HbF levels correlate with greater benefit for individuals with blood disease, such as reduction in disease symptoms and improved overall health. A T to C mutation at the −198 position in the HBG promoter causes HPFH by interference of binding to γ-globulin repressor proteins, such as BCL11A.

Ex vivo manipulation and/or editing of HSCs prior to administration to patients as a cell therapy is a promising approach for the treatment of hematological disorders. ABEs can introduce a T to C substitution at the −198 position of the promoter region of HBG1/2 (Gaudelli, N. M. et al. Programmable base editing of A*T to G*C in genomic DNA without DNA cleavage. Nature 551, 464-471, doi: 10.1038/nature24644 (2017)). This naturally occurring allele yields Hereditary Persistence of Fetal Hemoglobin (HPFH) resulting in increased levels of γ-globin into adulthood, which can mitigate the defects in β-globin seen in sickle cell disease and β-thalassemia (Wienert, B. et al. KLF1 drives the expression of fetal hemoglobin in British HPFH. Blood 130, 803-807, doi: 10.1182/blood-2017-02-767400 (2017)). Base editor systems have been developed that significantly increase the level of HbF following nucleotide conversion at key regulatory motifs within the HBG1 and HBG2 (HBG1/2) promoters.

Increasing levels of HbF expression leads to protection to the majority of SCD and β-thalassemia patients based on clinical observations of HPFH and non-interventional therapy that links higher HbF dosage with milder disease (Ngo et al., 2011 Brit J Hem, Vol. 156 (2): 259-264; Musallam et al., 2012 Blood). Accordingly, in the HPFH approach described here, base editing is used to recreate single base changes in the regulatory region of both gamma globin genes (HBG1 and HBG2) that disrupt repressor binding and lead to increased expression of fetal hemoglobin (HbF). Beta-thalassemia or sickle cell disease patients naturally harboring these variants are often asymptomatic or experience a milder form of the disease.

An exemplary target sequence for introducing an alteration to an HBG1/2 promoter to increase expression of fetal hemoglobin is provided below:

The target sequence, including edited bases 5 and 8 (in bold) and PAM (SEQ ID NO: 503):

| 1 | 2 | 3 | 4 | 5 | 6 | 7 | 8 | 9 | 10 | 11 | 12 | 13 | 14 | 15 | 16 | 17 | 18 | 19 | 20 | PAM |
|---|---|---|---|---|---|---|---|---|---|---|---|---|---|---|---|---|---|---|---|---|
| C | T | T | G | A | C | C | A | A | T | A | G | C | C | T | T | G | A | C | A | AGG |

### Methods of Treatment

The methods and compositions disclosed herein may be used to condition a subject's tissues (e.g., bone marrow) for engraftment or transplant and following such conditioning, a stem cell population is administered to the subject. The transplanted cells (e.g., HSCs) can be autologous cells or allogeneic cells. In certain aspects, the stem cell population comprises an exogenous stem cell population. In some embodiments, the stem cell population comprises the subject's endogenous stem cells (e.g., endogenous stem cells that have been genetically modified to correct a disease or genetic defect, such as those associated with sickle cell disease). Such methods and compositions may be useful for treating such diseases without causing the toxicities that are observed in response to traditional conditioning therapies, such as irradiation.

Hematopoietic stem cell transplant therapy can be administered to a subject in need of treatment so as to populate or re-populate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

Hematopoietic stem cells can thus be administered to a patient defective or deficient in one or more cell types of the hematopoietic lineage in order to reconstitute the defective or deficient population of cells in vivo, thereby treating the pathology associated with the defect or depletion in the endogenous blood cell population. The compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome). Additionally, or alternatively, the compositions and methods described herein can be used to treat a malignancy or proliferative disorder, such as a hematologic cancer, myeloproliferative disease. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Antibodies, antigen-binding fragments thereof, and ligands described herein can be administered to a patient (e.g., a human patient suffering from cancer, an autoimmune disease, or in need of hematopoietic stem cell transplant therapy) in a variety of dosage forms. For instance, antibodies, antigen-binding fragments thereof, and ligands described herein can be administered to a patient suffering from cancer, an autoimmune disease, or in need of hematopoietic stem cell transplant therapy in the form of an aqueous solution, such as an aqueous solution containing one or more pharmaceutically acceptable excipients. Pharmaceutically acceptable excipients for use with the compositions and methods described herein include viscosity-modifying agents. The aqueous solution may be sterilized using techniques known in the art. Non-limiting examples of antibodies suitable for use in the methods of the disclosure include ABTx025, ABTx030, ABTx052, ABTx061, ABTx062, ABTx070, ABTx071, JSP191, and MGTA-117. Further non-limiting examples of anti-CD117 antibodies useful in embodiments of the methods of the disclosure include 1C5, 2B8, 104D2, ACK2, JSP191, K45, Y45.B8, ST04-99, 22HCLC, G.813.2, 8D7, ACK4, 2B8/BM, OTI2B12, OTI1B6, OTI2E3, OTI1E2, OTI2CID5, OTI3F9, OTI9A11, OTI14B1, 6F2, UMAB216, OTI2B5, OTI1F6, OTI2C1H4, OTI6F8, and derivatives thereof available, for example, from ThermoFisher Scientific.

In various embodiments, an antibody of the disclosure is administered to a subject before, after, or concurrently with administration of transplanted cells (e.g., HSCs) to the subject. In embodiments, the methods of the disclosure involve administering cells (e.g., HSCs of the disclosure) to a subject previously that was previously administered an anti-CD117 antibody of the disclosure. In some cases, the blood of the subject contains a therapeutically effective amount of the anti-CD117 antibody at the time at which the cells are administered to the subject. The antibody of the disclosure may persist at a therapeutically effective level in a subject (e.g., in the blood of the subject) for about or at least about 1 day, 2 days, 3 days, 4 days, 5 days, 6 days, 7 days, 8 days, 1 wk, 2 wks, 3 wks, 4 wks, 5 wks, 10 wks, 15 wks, or 20 wks after administration of the antibody to the subject. An antibody or antigen binding fragment thereof, antibody drug conjugate, or a chimeric antigen receptor T (CAR-T) cell may be administered to a subject before, after, or concurrently with a edited cell of the disclosure.

IgG1 antibodies targeting CD117 can cause mast cell degranulation, which can result from Fcγ receptor interactions. Accordingly, in some embodiments an antibody (e.g., ABTx052) can be modified to include one or more amino acid residue alterations in an Fc domain to reduce or eliminate binding to FcγR, binding to FcRn, and/or mast cell activation. In embodiments, an antibody is modified to include amino acid alterations that prevent or reduce hypersensitivity reactions (HSRs). In some embodiments, an antibody (e.g., ABTx052) is modified to include an amino acid alteration at one or more of the following sites: L234, L235, G236, D265, N297, and P329. In some cases, the antibody includes an amino acid alteration and/or combination of amino acid alterations selected from: N297Q (aglycosylated); L234A and L235A (LALA); L234A, L235A, and P329G (LALAPG); L234A, L235A, and D265A (LALADA); L234S, L235T, and G236R (STR or LSLTGR); M252Y, S254T, and T256E (YTE); and LS M428L and N434S (LS) (see, e.g., Rosenberg, et al., PloS One, 14: e0212649, the disclosure of which is incorporated herein by reference in its entirety for all purposes); where the amino acid positions are referenced to the following amino acid sequence:

(positions 7-1101 of SEQ ID NO: 419)
SGAAVKKPGESLKISCKGSGYRFTSYWIGWVRQMPGKGLEWMGIIYPGDS
DTRYSPSFQGQVTISAGKSISTAYLOWSSLKASDTAMYYCARHGRGYNGY
EGAFDIWGQGTMVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG.

The antibodies, antigen-binding fragments, and ligands described herein may be administered by a variety of routes, such as orally, transdermally, subcutaneously, intranasally, intravenously, intramuscularly, intraocularly, or parenterally. The most suitable route for administration in any given case will depend on the particular antibody, antigen-binding fragment, or ligand administered, the patient, pharmaceutical formulation methods, administration methods (e.g., administration time and administration route), the patient's age, body weight, sex, severity of the diseases being treated, the patient's diet, and the patient's excretion rate.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Editing of Target Genes

To produce the gene edits described above, cells (e.g., hematopoietic stem cells (HSCs)) are contacted with one or more guide RNAs and a nucleobase editor polypeptide comprising a nucleic acid programmable DNA binding protein (napDNAbp) and an adenosine deaminase. In some cases, the cells are collected from a subject prior to the contacting. In some embodiments, the gRNA comprises nucleotide analogs. These nucleotide analogs can inhibit degradation of the gRNA from cellular processes. Tables 1 and 2 provides exemplary spacer sequences to be used for gRNAs. In some instances, a spacer sequence can be selected from those listed in Tables 1 and 2 or from a variant thereof with a truncation and/or extension (e.g., a 1, 2, 3, 4, 5, 6, 7, 8, 9, or 10 nucleotide 3′ and/or 5′ truncation and/or extension). In some instances, the gRNA is added directly to a cell.

Base editing can be carried out in vitro or in vivo. In some embodiments, cells (e.g., a hematopoietic stem cells (HSCs)) are collected from a subject or a donor. In some embodiments, base editing is carried out to induce changes in the genome of the cell. In some embodiments, base editing is carried out to induce changes in the genome of an allogeneic cell.

In some embodiments, cells of the present disclosure, are contacted with one or more guide RNAs and a nucleobase editor polypeptide comprising a nucleic acid programmable DNA binding protein (napDNAbp) (e.g., Cas9) domain and an adenosine deaminase domain. In some embodiments, the at least one nucleic acid molecule encoding one or more guide RNAs and a nucleobase editor polypeptide is delivered to cells by one or more vectors (e.g., AAV vector or lipid nanoparticle). In some cases, a guide RNA(s) and a nucleobase editor polypeptide is delivered to cells by electroporation.

The present disclosure provides one or more guide RNAs that direct a nucleobase editor polypeptide to edit a site in the genome of the cell (e.g., hematopoietic stem cell (HSC)). In some embodiments, the present disclosure provides guide RNAs that target a CD117 polynucleotide, a beta globin (HBB) polynucleotide, and/or a promoter region of a HBG1/2 polynucleotide. Exemplary guide RNA spacer sequences are provided in the below Tables 1 and 2.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

TABLE 1
Guide RNAs sequences

| | | SEQ | | SEQ | | |
|---|---|---|---|---|---|---|
| | | ID | | ID | | |
| Guide No. | Spacer | NO: | Target Sequence | NO: | PAM | Mutations |
| Guide-CC1 | AUGAAACGAAUG | 504 | ATGAAACGAATGAGA | 703 | CAG | E73G |
| | AGAAUAAG | | ATAAG | | | T74A |
| | | | | | | N75D |
| Guide-CC2 | UGGAUGAAACGA | 505 | TGGATGAAACGAATG | 704 | AAG | D72G |
| | AUGAGAAU | | AGAAT | | | E73G |
| | | | | | | T74A |
| Guide-CC3 | GCCAGGGGAAGC | 506 | GCCAGGGGAAGCCTC | 705 | AAG | Q152R |
| | CUCUUCCC | | TTCCC | | | K154E |
| Guide-CC4 | UCUGUGUACUCA | 507 | TCTGTGTACTCAACG | 706 | AAG | Y243C |
| | ACGUGGAA | | TGGAA | | | |
| Guide-CC5 | UGUGUACUCAAC | 508 | TGTGTACTCAACGTG | 707 | GAG | Y243C |
| | GUGGAAAA | | GAAAA | | | |
| Guide-CC6 | GGGAAGAUUAUC | 509 | GGGAAGATTATCCCA | 708 | GAG | E360G |
| | CCAAGUCU | | AGTCT | | | D361G |
| Guide-CC7 | CCAGAAAUCCUG | 510 | CCAGAAATCCTGACT | 709 | CAG | E414G |
| | ACUUACGA | | TACGA | | | I415V |
| Guide-CC8 | UCUGAGAAUGAA | 511 | TCTGAGAATGAAAGT | 710 | CAG | E366G |
| | AGUAAUAU | | AATAT | | | N367G |
| Guide-CC9 | CUAUUUUAACUU | 512 | CTATTTTAACTTTGC | 711 | AAG | Y503C |
| | UGCAUUUA | | ATTTA | | | N505G |
| Guide- | UUCACGAGCCUG | 513 | TTCACGAGCCTGTCG | 712 | CAG | L421P |
| CC10 | UCGUAAGU | | TAAGT | | | V422A |
| Guide- | CAGCAGUGGAUC | 514 | CAGCAGTGGATCTAT | 713 | CAG | Q347R |
| CC11 | UAUAUGAA | | ATGAA | | | |
| Guide- | CAUUCCCCAAAC | 515 | CATTCCCCAAACCTG | 714 | CAG | K342E |
| CC12 | CUGAACAC | | AACAC | | | |
| Guide- | UAUCCCAAGUCU | 516 | TATCCCAAGTCTGAG | 715 | AAG | K364G |
| CC13 | GAGAAUGA | | AATGA | | | |
| Guide- | AUAAAUGGGAAG | 517 | ATAAATGGGAAGATT | 716 | AAG | K358G |
| CC14 | AUUAUCCC | | ATCCC | | | |
| Guide- | UCCCCAAACCUG | 518 | TCCCCAAACCTGAAC | 717 | CAG | K342G |
| CC15 | AACACCAG | | ACCAG | | | |
| Guide- | GCCAUUCACGAG | 519 | GCCATTCACGAGCCT | 718 | AAG | V422A |
| CC16 | CCUGUCGU | | GTCGT | | | |
| Guide- | AAAUGUCACAAC | 520 | AAATGTCACAACAAC | 719 | AAG | N300S |
| CC17 | AACCUUGG | | CTTGG | | | T302A |
| Guide- | CGUGGAAAAGAG | 521 | CGTGGAAAAGAGAAA | 720 | CAG | K247G |
| CC18 | AAAACAGU | | ACAGT | | | R248G |
| Guide- | CCAUUUGACAAA | 522 | CCATTTGACAAAGCC | 721 | CAG | V64A |
| CC19 | GCCCGGAU | | CGGAT | | | W66R |
| Guide- | AGCAAGCUAUCU | 523 | AGCAAGCTATCTTCT | 722 | AAG | S220G |
| CC20 | UCUUAGGG | | TAGGG | | | Y221C |
| Guide- | UGAGGUUUAUUC | 524 | TGAGGTTTATTCCTG | 723 | AAG | R161G |
| CC21 | CUGACCCC | | ACCCC | | | I163V |
| Guide- | UGAAUGGAUCAC | 525 | TGAATGGATCACGGA | 724 | CAG | E81G I83V |
| CC22 | GGAAAAGG | | AAAGG | | | |
| Guide- | CCACUGGCAGUA | 526 | CCACTGGCAGTACAG | 725 | GAG | L455P |
| CC23 | CAGAAGCA | | AAGCA | | | V457A |
| Guide- | AGAAUGAAUGGA | 527 | AGAATGAATGGATCA | 726 | AAG | N80G |
| CC24 | UCACGGAA | | CGGAA | | | E81G |
| Guide- | UGUAAACGAUGG | 528 | TGTAAACGATGGAGA | 727 | TAG | N326G |
| CC25 | AGAAAAUG | | AAATG | | | D327G |
| Guide- | AGGCUUACAACG | 529 | AGGCTTACAACGATG | 728 | AAG | Y494C |
| CC26 | AUGUGGGC | | TGGGC | | | N495D |
| Guide- | AAGUGACCAAUU | 530 | AAGTGACCAATTATT | 729 | AAG | T144A |
| CC27 | AUUCCCUC | | CCCTC | | | N145D |
| Guide- | UGUCACAACAAC | 531 | TGTCACAACAACCTT | 730 | TAG | T302A |
| CC28 | CUUGGAAG | | GGAAG | | | T303A |
| Guide- | AAUAGAUUGGUA | 532 | AATAGATTGGTATTT | 731 | CAG | I438M |
| CC29 | UUUUUGUC | | TTGTC | | | D439G |
| Guide- | ACAAUGGCACGG | 533 | ACAATGGCACGGTTG | 732 | AAG | N486G |
| CC30 | UUGAAUGU | | AATGT | | | T488A |
| Guide- | ACACAACAGGCA | 534 | ACACAACAGGCACAG | 733 | AAG | V213A |
| CC31 | CAGCUUUG | | CTTTG | | | V214A |
| Guide- | GUUCAGAGUUCU | 535 | GTTCAGAGTTCTATA | 734 | TAG | Q475R |
| CC32 | AUAGAUUC | | GATTC | | | S476G |
| Guide- | AGAUUUGAUUGU | 536 | AGATTTGATTGTTGA | 735 | AAG | D332G |
| CC33 | UGAAUAUG | | ATATG | | | I334V |
| Guide- | AUAAACAUUAAA | 537 | ATAAACATTAAATGC | 736 | CAG | V407A |
| CC34 | UGCUAUGG | | TATGG | | | Y408H |
| Guide- | AUCCAUCCAUCC | 538 | ATCCATCCATCCAGG | 737 | CAG | I39V H40R |
| CC35 | AGGAAAAU | | AAAAT | | | |
| Guide- | AUGGAUCACGGA | 539 | ATGGATCACGGAAAA | 738 | AAG | I83V T84A |
| CC36 | AAAGGCAG | | GGCAG | | | |
| Guide- | GUUGACUAUCAG | 540 | GTTGACTATCAGTTC | 739 | GAG | T276A |
| CC37 | UUCAGCGA | | AGCGA | | | I277V |
| Guide- | CACAACAACCUU | 541 | CACAACAACCTTGGA | 740 | TAG | T303A |
| CC38 | GGAAGUAG | | AGTAG | | | T304A |
| Guide- | CUAUAGAUUCUA | 542 | CTATAGATTCTAGTG | 741 | AAG | I478V |
| CC39 | GUGCAUUC | | CATTC | | | D479G |
| Guide- | UGCCAAUAAUAC | 543 | TGCCAATAATACTTT | 742 | CAG | N293G |
| CC40 | UUUUGGAU | | TGGAT | | | N294G |
| Guide- | GCUUUGUCAAAU | 544 | GCTTTGTCAAATGGA | 743 | GAG | K65E |
| CC41 | GGACUUUU | | CTTTT | | | |
| Guide- | CUUCCCCAUGAU | 545 | CTTCCCCATGATAAA | 744 | CAG | M318V |
| CC42 | AAACACUA | | CACTA | | | |
| Guide- | CGUUUGGAAAGC | 546 | CGTTTGGAAAGCTAG | 745 | CAG | K471E |
| CC43 | UAGUGGUU | | TGGTT | | | |
| Guide- | AAACUACAGGAG | 547 | AAACTACAGGAGAAA | 746 | TAG | Q256R |
| CC44 | AAAUAUAA | | TATAA | | | |
| Guide- | AGGAGGCACUUA | 548 | AGGAGGCACTTACAC | 747 | TAG | T389A |
| CC45 | CACAUUCC | | ATTCC | | | |
| Guide- | CUUGAAUGCACU | 549 | CTTGAATGCACTAGA | 748 | TAG | F483P |
| CC46 | AGAAUCUA | | ATCTA | | | |
| Guide- | UUCCAAACGGUG | 550 | TTCCAAACGGTGGCC | 749 | GAG | F469P |
| CC47 | GCCCAGAU | | CAGAT | | | |
| Guide- | UCCAGGAAAAUC | 551 | TCCAGGAAAATCAGA | 750 | TAG | K43G |
| CC48 | AGACUUAA | | CTTAA | | | |
| Guide- | CCCUCAAGGGGU | 552 | CCCTCAAGGGGTGCC | 751 | AAG | K149G |
| CC49 | GCCAGGGG | | AGGGG | | | |
| Guide- | UCCAAAGCAAGC | 553 | TCCAAAGCAAGCTAT | 752 | TAG | K218G |
| CC50 | UAUCUUCU | | CTTCT | | | |
| Guide- | UAGGGAAGGGGA | 554 | TAGGGAAGGGGAAGA | 753 | CAG | E225G |
| CC51 | AGAAUUCA | | ATTCA | | | |
| Guide- | AGCGAGAGUUAA | 555 | AGCGAGAGTTAATGA | 754 | GAG | R281G |
| CC52 | UGAUUCUG | | TTCTG | | | |
| Guide- | UUUGGAAAGCUA | 556 | TTTGGAAAGCTAGTG | 755 | GAG | K471G |
| CC53 | GUGGUUCA | | GTTCA | | | |
| Guide- | AGACGGUUCCCC | 557 | AGACGGTTCCCCTGG | 756 | CAG | S35P |
| CC54 | UGGACUCA | | ACTCA | | | |
| Guide- | ACGUUGACUAUC | 558 | ACGTTGACTATCAGT | 757 | GAG | T276A |
| CC55 | AGUUCAGC | | TCAGC | | | |
| Guide- | CUGCACAUCCAC | 559 | CTGCACATCCACTGG | 758 | CAG | V459A |
| CC56 | UGGCAGUA | | CAGTA | | | |
| Guide- | CACAUCCACUGG | 560 | CACATCCACTGGCAG | 759 | AAG | V457A |
| CC57 | CAGUACAG | | TACAG | | | |
| Guide- | AUCCACUGGCAG | 561 | ATCCACTGGCAGTAC | 760 | CAG | V457A |
| CC58 | UACAGAAG | | AGAAG | | | |
| Guide- | UUGACGUCAGAA | 562 | TTGACGTCAGAATTG | 761 | TAG | V399A |
| CC59 | UUGGACAC | | GACAC | | | |
| Guide- | AUUAACUCUCGC | 563 | ATTAACTCTCGCTGA | 762 | TAG | V282A |
| CC60 | UGAACUGA | | ACTGA | | | |
| Guide- | CCAUACAAGGAG | 564 | CCATACAAGGAGCGG | 763 | AAG | L124P |
| CC61 | CGGUCAAC | | TCAAC | | | Y125H |
| Guide- | UUCAGGAUGAAU | 565 | TTCAGGATGAATTTT | 76 | CAG | I201T |
| CC62 | UUUUCCGA | | TCCGA | | | L202P |
| Guide- | GUGACUUCAAUU | 566 | GTGACTTCAATTATG | 765 | CAG | D266G |
| CC63 | AUGAACGU | | AACGT | | | N268D |
| Guide- | GUCGGAAAAAUU | 567 | GTCGGAAAAATTCAT | 766 | AAG | E198G |
| CC64 | CAUCCUGA | | CCTGA | | | K199G |
| Guide- | AUUAUAUUUCUC | 568 | ATTATATTTCTCCTG | 767 | TAG | Y259H |
| CC65 | CUGUAGUU | | TAGTT | | | |
| Guide- | AAGGAGCGGUCA | 569 | AAGGAGCGGTCAACA | 768 | AAG | S123P |
| CC66 | ACAAGGAA | | AGGAA | | | |
| Guide- | GAGAUUAAAAGG | 570 | GAGATTAAAAGGCAC | 769 | GAG | K383G |
| CC67 | CACCGAAG | | CGAAG | | | |
| Guide- | AACGAGAUUAAA | 571 | AACGAGATTAAAAGG | 770 | AAG | R381G |
| CC68 | AGGCACCG | | CACCG | | | |
| Guide- | GAAAAAUUCAUC | 572 | GAAAAATTCATCCTG | 771 | GAG | K199G |
| CC69 | CUGAAAGU | | AAAGT | | | |
| Guide- | AUUCAUCCUGAA | 573 | ATTCATCCTGAAAGT | 772 | CAG | I201V |
| CC70 | AGUGAGGC | | GAGGC | | | |
| Guide- | GAAAGAAGACAA | 574 | GAAAGAAGACAACGA | 773 | TGG | K127R |
| CC71 | CGACACGC | | CACGC | | | E128G |
| | | | | | | D129G |
| Guide- | UACAGGAGAAAU | 575 | TACAGGAGAAATATA | 774 | TGG | Q256R |
| CC72 | AUAAUAGC | | ATAGC | | | E257G |
| | | | | | | K258E |
| Guide- | AUGAGAAUAAGC | 576 | ATGAGAATAAGCAGA | 775 | TGG | E76G |
| CC73 | AGAAUGAA | | ATGAA | | | N77G |
| | | | | | | K78E |
| Guide- | CCAGGGGAAGCC | 577 | CCAGGGGAAGCCTCT | 776 | AGG | Q152R |
| CC74 | UCUUCCCA | | TCCCA | | | K154G |
| Guide- | CAGAAAUCCUGA | 578 | CAGAAATCCTGACTT | 777 | AGG | E414G |
| CC75 | CUUACGAC | | ACGAC | | | I415V |
| Guide- | ACAGAACCUUCA | 579 | ACAGAACCTTCACTG | 778 | TGG | R353G |
| CC76 | CUGAUAAA | | ATAAA | | | T354A |
| Guide- | UCACGAGCCUGU | 580 | TCACGAGCCTGTCGT | 779 | AGG | L421P |
| CC77 | CGUAAGUC | | AAGTC | | | V422A |
| Guide- | ACUUACGACAGG | 581 | ACTTACGACAGGCTC | 780 | TGG | Y418C |
| CC78 | CUCGUGAA | | GTGAA | | | D419G |
| Guide- | UGUUCAUAUAGA | 582 | TGTTCATATAGATCC | 781 | TGG | M351T |
| CC79 | UCCACUGC | | ACTGC | | | |
| Guide- | CCAAACCUGAAC | 583 | CCAAACCTGAACACC | 782 | TGG | K342G |
| CC80 | ACCAGCAG | | AGCAG | | | |
| Guide- | CUGUCGUAAGUC | 584 | CTGTCGTAAGTCAGG | 783 | TGG | Y418H |
| CC81 | AGGAUUUC | | ATTTC | | | |
| Guide- | CAGAACCUUCAC | 585 | CAGAACCTTCACTGA | 784 | GGG | T354A |
| CC82 | UGAUAAAU | | TAAAT | | | |
| Guide- | UAUUUUAACUUU | 586 | TATTTTAACTTTGCA | 785 | AGG | N505G |
| CC83 | GCAUUUAA | | TTTAA | | | |
| Guide- | UAAGCAGAAUGA | 587 | TAAGCAGAATGAATG | 786 | CGG | K78R |
| CC84 | AUGGAUCA | | GATCA | | | Q79R |
| | | | | | | N80D |
| Guide- | GAAUGGCAUGCU | 588 | GAATGGCATGCTCCA | 787 | TGG | N423S |
| CC85 | CCAAUGUG | | ATGTG | | | M425V |
| Guide- | GUGCAGACACUA | 589 | GTGCAGACACTAAAC | 788 | TGG | Q460R |
| CC86 | AACUCAUC | | TCATC | | | T461A |
| Guide- | UGACCAAUUAUU | 590 | TGACCAATTATTCCC | 789 | GGG | T144A |
| CC87 | CCCUCAAG | | TCAAG | | | N145G |
| Guide- | GUGACCAAUUAU | 591 | GTGACCAATTATTCC | 790 | GGG | T144A |
| CC88 | UCCCUCAA | | CTCAA | | | N145G |
| Guide- | AGUGACCAAUUA | 592 | AGTGACCAATTATTC | 791 | AGG | T144A |
| CC89 | UUCCCUCA | | CCTCA | | | N145G |
| Guide- | GAAUGAAUGGAU | 593 | GAATGAATGGATCAC | 792 | AGG | N80S |
| CC90 | CACGGAAA | | GGAAA | | | E81G |
| Guide- | UGCAGACACUAA | 594 | TGCAGACACTAAACT | 793 | GGG | Q460R |
| CC91 | ACUCAUCU | | CATCT | | | T461A |
| Guide- | CCAAAGCAAGCU | 595 | CCAAAGCAAGCTATC | 794 | AGG | K218G |
| CC92 | AUCUUCUU | | TTCTT | | | S220G |
| Guide- | ACAGCACUGACU | 596 | ACAGCACTGACTTGC | 795 | TGG | V195A |
| CC93 | UGCCCUCC | | CCTCC | | | L196P |
| Guide- | GGAGGGCAAGUC | 597 | GGAGGGCAAGTCAGT | 796 | CGG | E191G |
| CC94 | AGUGCUGU | | GCTGT | | | K193G |
| Guide- | CACAUAAACAUU | 598 | CACATAAACATTAAA | 797 | TGG | V407A |
| CC95 | AAAUGCUA | | TGCTA | | | Y408H |
| Guide- | CAAAUGGACUUU | 599 | CAAATGGACTTTTGA | 798 | TGG | K65R |
| CC96 | UGAGAUCC | | GATCC | | | T67A |
| Guide- | CAAUGGCACGGU | 600 | CAATGGCACGGTTGA | 799 | AGG | N486S |
| CC97 | UGAAUGUA | | ATGTA | | | T488A |
| Guide- | AAAUACUGUAGU | 601 | AAATACTGTAGTGTT | 800 | TGG | V323A |
| CC98 | GUUUAUCA | | TATCA | | | F324L |
| Guide- | CAAAGCAAGCUA | 602 | CAAAGCAAGCTATCT | 801 | GGG | K218R |
| CC99 | UCUUCUUA | | TCTTA | | | S220G |
| Guide- | GGAAUAAACCUC | 603 | GGAATAAACCTCAAG | 802 | GGG | F162P |
| CC100 | AAGUCCUU | | TCCTT | | | I163T |
| Guide- | ACCACUAGCUUU | 604 | ACCACTAGCTTTCCA | 803 | TGG | L472P |
| CC101 | CCAAACGG | | AACGG | | | V473A |
| Guide- | GCAAGCUAUCUU | 605 | GCAAGCTATCTTCTT | 804 | AGG | S220G |
| CC102 | CUUAGGGA | | AGGGA | | | Y221C |
| Guide- | CAAGCUAUCUUC | 606 | CAAGCTATCTTCTTA | 805 | GGG | S220G |
| CC103 | UUAGGGAA | | GGGAA | | | Y221C |
| Guide- | UUUGAUCAUGAU | 607 | TTTGATCATGATGCC | 80€ | TGG | M171T |
| CC104 | GCCCGCCU | | CGCCT | | | I172T |
| Guide- | UUGAUCAUGAUG | 608 | TTGATCATGATGCCC | 807 | GGG | M171T |
| CC105 | CCCGCCUU | | GCCTT | | | I172T |
| Guide- | AUAGAUUGGUAU | 609 | ATAGATTGGTATTTT | 808 | AGG | I438M |
| CC106 | UUUUGUCC | | TGTCC | | | D439G |
| Guide- | AGGAAUAAACCU | 610 | AGGAATAAACCTCAA | 809 | TGG | F162P |
| CC107 | CAAGUCCU | | GTCCT | | | I163T |
| Guide- | CACAACAGGCAC | 611 | CACAACAGGCACAGC | 810 | AGG | V213A |
| CC108 | AGCUUUGA | | TTTGA | | | |
| Guide- | UGACGUCAGAAU | 612 | TGACGTCAGAATTGG | 811 | AGG | V399A |
| CC109 | UGGACACU | | ACACT | | | |
| Guide- | ACUACAGUAUUU | 613 | ACTACAGTATTTGTA | 812 | TGG | T322A |
| CC110 | GUAAACGA | | AACGA | | | |
| Guide- | AGCAAAUGUCAC | 614 | AGCAAATGTCACAAC | 813 | TGG | N300G |
| CC111 | AACAACCU | | AACCT | | | |
| Guide- | GUCUGAUUUUCC | 615 | GTCTGATTTTCCTGG | 814 | TGG | S44P |
| CC112 | UGGAUGGA | | ATGGA | | | |
| Guide- | UGCUGAUCCAAA | 616 | TGCTGATCCAAAAGT | 815 | TGG | S298P |
| CC113 | AGUAUUAU | | ATTAT | | | |
| Guide- | AUCAGACUUAAU | 617 | ATCAGACTTAATAGT | 816 | TGG | D45G |
| CC114 | AGUCCGCG | | CCGCG | | | |
| Guide- | UGCAUUCAAGCA | 618 | TGCATTCAAGCACAA | 817 | CGG | K484G |
| CC115 | CAAUGGCA | | TGGCA | | | |
| Guide- | UCCCAGAGCCCA | 619 | TCCCAGAGCCCACAA | 818 | TGG | E435G |
| CC116 | CAAUAGAU | | TAGAT | | | |
| Guide- | UCAGCGAGAGUU | 620 | TCAGCGAGAGTTAAT | 819 | TGG | R281G |
| CC117 | AAUGAUUC | | GATTC | | | |
| Guide- | GUGUGAAACGCG | 621 | GTGTGAAACGCGCCT | 820 | CGG | K176G |
| CC118 | CCUACCAU | | ACCAT | | | |
| Guide- | UCAGACUUAAUA | 622 | TCAGACTTAATAGTC | 821 | GGG | D45G |
| CC119 | GUCCGCGU | | CGCGT | | | |
| Guide- | UCUAGUGCAUUC | 623 | TCTAGTGCATTCAAG | 822 | TGG | S481G |
| CC120 | AAGCACAA | | CACAA | | | |
| Guide- | AUUCCCUCAAGG | 624 | ATTCCCTCAAGGGGT | 823 | GGG | K149E |
| CC121 | GGUGCCAG | | GCCAG | | | |
| Guide- | AAGGCAGAAGCC | 625 | AAGGCAGAAGCCACC | 824 | CGG | E88G |
| CC122 | ACCAACAC | | AACAC | | | |
| Guide- | GAGGUUUAUUCC | 626 | GAGGTTTATTCCTGA | 825 | AGG | I163V |
| CC123 | UGACCCCA | | CCCCA | | | |
| Guide- | UUUAUUCCUGAC | 627 | TTTATTCCTGACCCC | 826 | GGG | I163V |
| CC124 | CCCAAGGC | | AAGGC | | | |
| Guide- | GUUUAUUCCUGA | 628 | GTTTATTCCTGACCC | 827 | CGG | I163V |
| CC125 | CCCCAAGG | | CAAGG | | | |
| Guide- | CAUACAAGGAGC | 629 | CATACAAGGAGCGGT | 828 | AGG | L124P |
| CC126 | GGUCAACA | | CAACA | | | Y125H |
| Guide- | AAAAAUUCAUCC | 630 | AAAAATTCATCCTGA | 829 | AGG | K199G |
| CC127 | UGAAAGUG | | AAGTG | | | I201V |
| Guide- | AAAUAUAAUAGC | 631 | AAATATAATAGCTGG | 830 | CGG | Y259C |
| CC128 | UGGCAUCA | | CATCA | | | N260D/G/ |
| | | | | | | S |
| Guide- | UGACUUCAAUUA | 632 | TGACTTCAATTATGA | 831 | AGG | D266G |
| CC129 | UGAACGUC | | ACGTC | | | N268G |
| Guide- | CUAGUUCUGUGU | 633 | CTAGTTCTGTGTACT | 832 | TGG | S240G |
| CC130 | ACUCAACG | | CAACG | | | |
| Guide- | AGAUUAAAAGGC | 634 | AGATTAAAAGGCACC | 833 | AGG | K383G |
| CC131 | ACCGAAGG | | GAAGG | | | |
| Guide- | ACGAGAUUAAAA | 635 | ACGAGATTAAAAGGC | 834 | AGG | R381G |
| CC132 | GGCACCGA | | ACCGA | | | |
| Guide- | GGAUGAAACGAA | 636 | GGATGAAACGAATGA | 835 | AGC | E73G |
| CC133 | UGAGAAUA | | GAATA | | | T74A |
| | | | | | | N75G |
| Guide- | AACUACAGGAGA | 637 | AACTACAGGAGAAAT | 836 | AGC | Q256R |
| CC134 | AAUAUAAU | | ATAAT | | | E257G |
| | | | | | | K258G |
| Guide- | UGGGAAAGAAGA | 638 | TGGGAAAGAAGACAA | 837 | CGC | K127G |
| CC135 | CAACGACA | | CGACA | | | E128G |
| | | | | | | D129G |
| Guide- | GCCAAUAAUACU | 639 | GCCAATAATACTTTT | 838 | AGC | N293G |
| CC136 | UUUGGAUC | | GGATC | | | N294G |
| | | | | | | T295A |
| Guide- | CUUACGACAGGC | 640 | CTTACGACAGGCTCG | 839 | GGC | Y418C |
| CC137 | UCGUGAAU | | TGAAT | | | D419G |
| | | | | | | R420G |
| Guide- | GUUAAAAUAGGC | 641 | GTTAAAATAGGCAGA | 840 | TGC | Y503H |
| CC138 | AGAAGUCU | | AGTCT | | | F504P |
| Guide- | UUCUGUUCAUAU | 642 | TTCTGTTCATATAGA | 841 | TGC | Y350H |
| CC139 | AGAUCCAC | | TCCAC | | | M351T |
| Guide- | AGAAAUCCUGAC | 643 | AGAAATCCTGACTTA | 842 | GGC | I415V |
| CC140 | UUACGACA | | CGACA | | | T417A |
| Guide- | AACGAUGUGGGC | 644 | AACGATGTGGGCAAG | 843 | TGC | D496G |
| CC141 | AAGACUUC | | ACTTC | | | K499E |
| Guide- | AUUCCCCAAACC | 645 | ATTCCCCAAACCTGA | 844 | AGC | K342G |
| CC142 | UGAACACC | | ACACC | | | |
| Guide- | CGACAGGCUCGU | 646 | CGACAGGCTCGTGAA | 845 | TGC | R420G |
| CC143 | GAAUGGCA | | TGGCA | | | |
| Guide- | ACUUCUGCCUAU | 647 | ACTTCTGCCTATTTT | 846 | TGC | Y503C |
| CC144 | UUUAACUU | | AACTT | | | |
| Guide- | GCGACGAGAUUA | 648 | GCGACGAGATTAGGC | 847 | TGC | D52G |
| CC145 | GGCUGUUA | | TGTTA | | | E53G I54V |
| | | | | | | R55G |
| Guide- | AAGACAACGACA | 649 | AAGACAACGACACGC | 848 | CGC | D129G |
| CC146 | CGCUGGUC | | TGGTC | | | N130G |
| | | | | | | D131G |
| | | | | | | T132A |
| Guide- | UGGAUCACGGAA | 650 | TGGATCACGGAAAAG | 849 | AGC | I83V T84A |
| CC147 | AAGGCAGA | | GCAGA | | | E85G |
| | | | | | | K86E |
| Guide- | GACACAGACACA | 651 | GACACAGACACAACA | 850 | AGC | V213A |
| CC148 | ACAGGCAC | | GGCAC | | | V214A |
| | | | | | | S215P |
| | | | | | | V216A |
| Guide- | GGGAAGAAUUCA | 652 | GGGAAGAATTCACAG | 851 | TGC | E227G |
| CC149 | CAGUGACG | | TGACG | | | E228G |
| | | | | | | T230A |
| Guide- | GCACCAACAAAC | 653 | GCACCAACAAACACG | 852 | AGC | N99G |
| CC150 | ACGGCUUA | | GCTTA | | | K100G |
| | | | | | | H101R |
| Guide- | UAUAGAUUCUAG | 654 | TATAGATTCTAGTGC | 853 | AGC | I478M |
| CC151 | UGCAUUCA | | ATTCA | | | D479G |
| | | | | | | S481G |
| Guide- | UCAUGAUCAAAA | 655 | TCATGATCAAAAGTG | 854 | CGC | I172V |
| CC152 | GUGUGAAA | | TGAAA | | | K173G |
| | | | | | | S174G |
| Guide- | AUGAUCAAAAGU | 656 | ATGATCAAAAGTGTG | 855 | CGC | I172V |
| CC153 | GUGAAACG | | AAACG | | | K173G |
| | | | | | | S174G |
| Guide- | AAUGAAUGGAUC | 657 | AATGAATGGATCACG | 856 | GGC | E81G I83V |
| CC154 | ACGGAAAA | | GAAAA | | | T84A |
| Guide- | CCAAUUAUUCCC | 658 | CCAATTATTCCCTCA | 857 | TGC | N145S |
| CC155 | UCAAGGGG | | AGGGG | | | Y146C |
| Guide- | CAGACUUAAUAG | 659 | CAGACTTAATAGTCC | 858 | GGC | D45G |
| CC156 | UCCGCGUG | | GCGTG | | | I47V |
| Guide- | UCUGACGUCAAU | 660 | TCTGACGTCAATGCT | 859 | AGC | D398G |
| CC157 | GCUGCCAU | | GCCAT | | | N400G |
| Guide- | GCAUAACAGCCU | 661 | GCATAACAGCCTAAT | 860 | CGC | L56P L57P |
| CC158 | AAUCUCGU | | CTCGT | | | |
| Guide- | UUUUUCCGACAG | 662 | TTTTTCCGACAGCAC | 861 | TGC | L196P |
| CC159 | CACUGACU | | TGACT | | | S197P |
| Guide- | UUUCCGUGAUCC | 663 | TTTCCGTGATCCATT | 862 | TGC | W82R |
| CC160 | AUUCAUUC | | CATTC | | | I83T |
| Guide- | UUUGACAAAGCC | 664 | TTTGACAAAGCCCGG | 863 | TGC | F63P |
| CC161 | CGGAUCAG | | ATCAG | | | V64A |
| Guide- | GCCCAGAUGAGU | 665 | GCCCAGATGAGTTTA | 864 | TGC | S464P |
| CC162 | UUAGUGUC | | GTGTC | | | S465P |
| Guide- | GCUGAACUGAUA | 666 | GCTGAACTGATAGTC | 865 | TGC | I277T |
| CC163 | GUCAACGU | | AACGT | | | S279P |
| Guide- | ACACUUUUGAUC | 667 | ACACTTTTGATCATG | 866 | CGC | M171T |
| CC164 | AUGAUGCC | | ATGCC | | | I172T |
| Guide- | GAAAAUCAGACU | 668 | GAAAATCAGACTTAA | 867 | CGC | K43R |
| CC165 | UAAUAGUC | | TAGTC | | | D45G |
| Guide- | GCAGACACUAAA | 669 | GCAGACACTAAACTC | 868 | GGC | T461A |
| CC166 | CUCAUCUG | | ATCTG | | | N463G |
| Guide- | GGACCAGGAGGG | 670 | GGACCAGGAGGGCAA | 869 | TGC | Q190R |
| CC167 | CAAGUCAG | | GTCAG | | | E191G |
| Guide- | GCUGAUCCAAAA | 671 | GCTGATCCAAAAGTA | 870 | GGC | F296P |
| CC168 | GUAUUAUU | | TTATT | | | S298P |
| Guide- | ACAUCCACUGGC | 672 | ACATCCACTGGCAGT | 871 | AGC | L455P |
| CC169 | AGUACAGA | | ACAGA | | | V457A |
| Guide- | CACUGGCAGUAC | 673 | CACTGGCAGTACAGA | 872 | AGC | V454A |
| CC170 | AGAAGCAG | | AGCAG | | | L455P |
| Guide- | AGGCAGAAGCCA | 674 | AGGCAGAAGCCACCA | 873 | GGC | E88G |
| CC171 | CCAACACC | | ACACC | | | T90A |
| Guide- | AGGUUUAUUCCU | 675 | AGGTTTATTCCTGAC | 874 | GGC | I163V |
| CC172 | GACCCCAA | | CCCAA | | | |
| Guide- | CCAAGCUUUUCC | 676 | CCAAGCTTTTCCTTG | 875 | CGC | K116R |
| CC173 | UUGUUGAC | | TTGAC | | | |
| Guide- | UUAGUGUCUGCA | 677 | TTAGTGTCTGCACAT | 876 | GGC | V459A |
| CC174 | CAUCCACU | | CCACT | | | |
| Guide- | CCACUAGCUUUC | 678 | CCACTAGCTTTCCAA | 877 | GGC | L472P |
| CC175 | CAAACGGU | | ACGGT | | | |
| Guide- | CCUCAAGGGGUG | 679 | CCTCAAGGGGTGCCA | 878 | AGC | K149G |
| CC176 | CCAGGGGA | | GGGGA | | | |
| Guide- | UGUGAAACGCGC | 680 | TGTGAAACGCGCCTA | 879 | GGC | K176G |
| CC177 | CUACCAUC | | CCATC | | | |
| Guide- | CUAGUGCAUUCA | 681 | CTAGTGCATTCAAGC | 880 | GGC | K484G |
| CC178 | AGCACAAU | | ACAAT | | | |
| Guide- | CCGCGUGGGCGA | 682 | CCGCGTGGGCGACGA | 881 | GGC | D52G |
| CC179 | CGAGAUUA | | GATTA | | | |
| Guide- | UUAUUCCUGACC | 683 | TTATTCCTGACCCCA | 882 | GGC | D165G |
| CC180 | CCAAGGCG | | AGGCG | | | |
| Guide- | ACUCCAGAAUCA | 684 | ACTCCAGAATCATTA | 883 | CGC | S285P |
| CC181 | UUAACUCU | | ACTCT | | | |
| Guide- | GGCUGUUAUGCA | 685 | GGCTGTTATGCACTG | 884 | GGC | T59A |
| CC182 | CUGAUCCG | | ATCCG | | | |
| Guide- | UUCAUCUAACGA | 686 | TTCATCTAACGAGAT | 885 | GGC | H378R |
| CC183 | GAUUAAAA | | TAAAA | | | T380A |
| | | | | | | R381G |
| Guide- | ACAGGAGAAAUA | 687 | ACAGGAGAAATATAA | 886 | GGC | E257G |
| CC184 | UAAUAGCU | | TAGCT | | | K258G |
| | | | | | | Y259C |
| Guide- | UCAGGAUGAAUU | 688 | TCAGGATGAATTTTT | 887 | AGC | F200P |
| CC185 | UUUCCGAC | | CCGAC | | | I201T |
| Guide- | GACUUCAAUUAU | 689 | GACTTCAATTATGAA | 888 | GGC | N268G |
| CC186 | GAACGUCA | | CGTCA | | | Y269C |
| Guide- | AGGAGCGGUCAA | 690 | AGGAGCGGTCAACAA | 889 | AGC | V120A |
| CC187 | CAAGGAAA | | GGAAA | | | S123P |
| Guide- | GAUUAAAAGGCA | 691 | GATTAAAAGGCACCG | 890 | GGC | K383G |
| CC188 | CCGAAGGA | | AAGGA | | | T385A |
| Guide- | AAAAUUCAUCCU | 692 | AAAATTCATCCTGAA | 891 | GGC | I201V |
| CC189 | GAAAGUGA | | AGTGA | | | |
| gRNA931; | AUAAUAGCUGGC | 693 | ATAATAGCTGGCATC | 892 | GACT | S261G |
| gRNA2342; | AUCACGGU | | ACGGT | | | N260D |
| gRNA2343; | | | | | | |
| CC200 | | | | | | |
| gRNA889 | CCACUAGCUUUC | 694 | CCACTAGCTTTCCAA | 893 | GGCC | L472P |
| | CAAACGGU | | ACGGT | | | |
| gRNA908 | GCUGAACUGAUA | 695 | GCTGAACTGATAGTC | 894 | TGCC | S279P |
| | GUCAACGU | | AACGT | | | |
| gRNA918 | UUUGACAAAGCC | 696 | TTTGACAAAGCCCGG | 895 | TGCA | V64A |
| | CGGAUCAG | | ATCAG | | | |
| gRNA923 | UGAAAGUGAGGC | 697 | TGAAAGTGAGGCCAG | 896 | GGCT | Silent |
| | CAGGUACU | | GTACT | | | |
| gRNA928 | AAACAGUCAGGU | 698 | AAACAGTCAGGTGAG | 897 | CGCT | S251G |
| | GAGUGAAU | | TGAAT | | | |
| gRNA929 | AACUACAGGAGA | 699 | AACTACAGGAGAAAT | 898 | AGCT | Silent |
| | AAUAUAAU | | ATAAT | | | |
| gRNA944 | GAUUAAAAGGCA | 700 | GATTAAAAGGCACCG | 899 | GGCA | Silent |
| | CCGAAGGA | | AAGGA | | | |
| gRNA291 | GGCUUUACCUCC | 701 | GGCTTTACCTCCAAT | 900 | AGG | A to G at |
| | AAUGGUGC | | GGTGC | | | splice site |
| | | | | | | (KO) |
| gRNA295 | CAGUACCUGGCC | 702 | CAGTACCTGGCCTCA | 901 | AGG | A to G at |
| | UCACUUUC | | CTTTC | | | splice site |
| | | | | | | (KO) |

TABLE 2
Exemplary Target Sequences. In embodiments, −198 or −114 refers to the
nucleotide 198 or 114 nucleotides upstream of the first transcribe nucleotide of HBG1/2. In
some instances, the site −114 is alternatively referred to as the site −115. In the table the
abbreviation “c.” indicates that position +1 is the first nucleotide transcribed and −1 is the
last nucleotide 5′ of the first transcribed nucleotide.

| Guide | | Description | | | SEQ | |
|---|---|---|---|---|---|---|
| Name | Gene | of Edit | Base Editor | gRNA Spacer Sequence | ID NO | PAM |
| sgRNA_ | HBB | Makassar | ABE | ACUUCUCCACAGGAGUCA | 902 | TGC |
| 017 | | | | GG | | |
| sgRNA_ | HBB | Makassar | ABE | UUCUCCACAGGAGUCAGG | 941 | |
| 027 | | | | UG | | |
| | HBG1/2 | c. −198 T > C | ABE | GUGGGGAAGGGGCCCCCA | 903 | AGG |
| | | | | AG | | |
| | HBG1/2 | c. −198 T > C | ABE | AUUGAGAUAGUGUGGGGA | 904 | GGG |
| | | | | AG | | |
| | HBG1/2 | c. −198 T > C | ABE | CAUUGAGAUAGUGUGGGG | 905 | GGG |
| | | | | AA | | |
| | HBG1/2 | c. −198 T > C | ABE | GCAUUGAGAUAGUGUGGG | 906 | AGG |
| | | | | GA | | |
| | HBG1/2 | c. −198 T > C | ABE | GUGGGGAAGGGGCCCCCA | 907 | AGG |
| | | | | AG | | |
| | HBG1/2 | c. −114 ~ | CBE and/or | GCUAUUGGUCAAGGCAAG | 908 | TGG |
| | | −102 deletion | ABE | GC | | |
| | HBG1/2 | c. −114 ~ | CBE and/or | CAAGGCUAUUGGUCAAGG | 909 | AGG |
| | | −102 deletion | ABE | CA | | |
| | HBG1/2 | c. −114 ~ | CBE and/or | CUUGUCAAGGCUAUUGGU | 910 | AGG |
| | | −102 deletion | ABE | CA | | |
| sgRNA_ | HBG1/2 | c. −114 ~ | CBE and/or | CUUGACCAAUAGCCUUGA | 911 | AGG |
| 015 | | −102 deletion | ABE | CA | | |
| (TSBTx045) | HBG1/2 | c. −114 ~ | CBE and/or | GUUUGCCUUGUCAAGGCU | 912 | TGG |
| | | −102 deletion | ABE | AU | | |
| | HBG1/2 | c. −114 ~ | CBE and/or | UGGUCAAGUUUGCCUUGU | 913 | AGG |
| | | 102 deletion | ABE | CA | | |
| | HBG1/2 | c. −198 T > C | ABE | UGGGGAAGGGGCCCCCAA | 914 | GGA |
| | | | | GA | | |
| | HBG1/2 | c. −198 T > C | ABE | GUGUGGGGAAGGGGCCCC | 915 | AGA |
| | | | | CA | | |
| | HBG1/2 | c. −175 T > C | ABE | UCAGACAGAUAUUUGCAU | 916 | AGA |
| | | | | UG | | |
| | HBG1/2 | c. −175 T > C | ABE | UUUCAGACAGAUAUUUGC | 917 | TGA |
| | | | | AU | | |
| | HBG1/2 | c. −114 ~ | CBE and/or | CUUGCCUUGACCAAUAGC | 918 | TGA |
| | | −102 deletion | ABE | CU | | |
| | HBG1/2 | c. −114 ~ | CBE and/or | UAGCCUUGACAAGGCAAA | 919 | TGA |
| | | −102 deletion | ABE | CU | | |
| | HBG1/2 | c. -90 | CBE and/or | CAAACUUGACCAAUAGUC | 920 | AGA |
| | | BCL11A | ABE | UU | | |
| | | binding | | | | |
| | HBG1/2 | c. −198 T > C | ABE | UGUGGGGAAGGGGCCCCC | 921 | GAGG |
| | | | | AA | | AT |
| | HBG1/2 | c. −202 | CBE and/or | GGGCCCCUUCCCCACACU | 922 | CTCA |
| | | C > T, −201 | ABE | AU | | AT |
| | | C > T, −198 | | | | |
| | | T > C, −197 | | | | |
| | | C > T, −196 | | | | |
| | | C > T, −195 | | | | |
| | | C > G | | | | |
| | HBG1/2 | c. −175 T > C | ABE | CAGACAGAUAUUUGCAUU | 923 | GATA |
| | | | | GA | | GT |
| | HBG1/2 | c. −175 T > C | ABE | UUUCAGACAGAUAUUUGC | 924 | TGAG |
| | | | | AU | | AT |
| | HBG1/2 | c. −114 ~ | CBE and/or | GCCUUGACAAGGCAAACU | 925 | ACCA |
| | | −102 deletion | ABE | UG | | AT |
| | HBG1/2 | c. −114 ~ | CBE and/or | UUGACAAGGCAAACUUGA | 926 | AATA |
| | | −102 deletion | ABE | CC | | GT |
| | HBG1/2 | c. -90 | CBE and/or | UGACCAAUAGUCUUAGAG | 927 | TCCA |
| | | BCL11A | ABE | UA | | GT |
| | | binding | | | | |
| | HBG1/2 | c. −175 T > C | ABE | AGACAGAUAUUUGCAUUG | 928 | TTT |
| | | | | AGAUA | | |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof. In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152 (5): 1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR (N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′ -NAA-3 ′ |

N is A, C, T, or G; and V is A, C, or G.

In some embodiments, the PAM is NGC. In some embodiments, the NGC PAM is recognized by a Cas9 variant. In some embodiments, the NGC PAM Cas9 variant includes one or more amino acid substitutions selected from D1135M, S1136Q, G1218K, E1219F, A1322R, D1332A, R1335E, and T1337R (collectively termed “MQKFRAER”) of spCas9 (SEQ ID No: 197), or a corresponding mutation in another Cas9. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218). In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9. In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science. aba8853 (2020); Hu et al. “Evolved Cas9 variants with broad PAM compatibility and high DNA specificity,” Nature, 2018 Apr. 5, 556 (7699), 57-63; Miller et al., “Continuous evolution of SpCas9 variants compatible with non-G PAMs” Nat. Biotechnol., 2020 April; 38 (4): 471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp.

The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS) n (SEQ ID NO: 246), SGGSSGGS (SEQ ID NO: 330), (GGGGS) n (SEQ ID NO: 247), (G) n, (EAAAK) n (SEQ ID NO: 248), (GGS) n, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, 1156F, or a corresponding mutation in another ACTIVE 705096791v1 184 adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | M | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | M | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | M | R | L | N | T | V | L | N | F | S | V | N | Y | G | A | C | Y | R | V | F | N | K |
| TadA*6.6 | M | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | M | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
Adenosine Deaminase Variants. Alterations are referenced
to TadA*7.10. Additional details of TadA*9 adenosine
deaminases are described in International PCT Application
No. PCT/US2020/049975, which is incorporated herein
by reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K_V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K_V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises one or more of M1I, S2A, S2E, V4D, V4E, V4M, F6S, H8E, H8Y, E9Y, M12S, R13H, R13I, R13Y, T17L, T17S, L18A, L18E, A19N, R21N, K20K, K20R, R21A, G22P, W23D, R23H, W23G, W23Q, W23L, W23R, D24E, D24G, E25F, E25M, E25D, E25A, E25G, E25R, E25V, E25S, E25Y, R26D, R26E, R26G, R26N, R26Q, R26C, R26L, R26K, R26W, E27V, E27D, P29V, V30G, L34S, L34V, L36H, H36L, H36N, N37N, N37T, N37S, N38G, N38R, W45A, W45L, W45N, N46N, R46W, R46F, R46Q, R46M, R47A, R47Q, R47F, R47K, R47P, R47W, R47M, P48T, P48L, P48A, P48I, P48S, 149G, 149H, 149V, 149F, 149H, G50L, R51H, R51L, R5IN, L51W, R51Y, H52D, H52Y, D53P, P54C, P54T, A55H, T55A, A56E, A56S, E59A, E59G, E59I, E59Q, E59W, M61A, M61I, M61L, M61V, L63S, L63V, Q65V, G66C, G67D, G67L, G67V, L68Q, M70H, M70Q, L84F, M70V, M70L, E70A, M70V, Q71M, Q7IN, Q71L, Q71R, N72A, N72K, N72S, N72D, N72Y, Y73G, Y73I, Y73K, Y73R, Y73S, R74A, R74Q, R74G, R74K, R74L, R74N, I76D, I76F, 1761, 176N, I76T, I76Y, D77G, A78I, T79M, L80M, L80Y, V82A, V82S, V82G, V82T, L84E, L84F, L84Y, E85K, E85G, E85P, E85S, S87C, S87L, S87V, V88A, V88M, C90S, A91A, A91G, A91S, A91V, A91T, G92T, A93I, M94A, M94V, M94L, M94I, M94H, 195S, 195G, 195L, 195H, 195V, H96A, H96L, H96R, H96S, S97C, S97G, S97I, S97M, S97R, S97S, R98K, R98I, R98N, R98Q, G100R, G100V, R101V, R101R, V102A, V102F, V102I, V102V, D103A, F104G, D104N, F104V, F104I, F104L, A106T, V106Q, V106F, V106W, V106M, A106A, A106Q, A106F, A106G, A106W, A106M, A106V, A106R, R107C, R107G, R107P, R107K, R107A, R107N, R107W, R107H, R107S, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, A109H, A109K, A109R, A109S, A109T, A109V, K110G, K110H, K110I, K11OR, K110T, T111A, T111G, T111H, T111R, G112A, A114G, A114H, A114V, G115S, L117M, L117N, L117V, M118D, M118G, M118K, M118N, M118V, D119L, D119N, D119S, D119V, V120H, V120L, H122H, H122N, H122P, H122R, H122S, H122Y, H123C, H123G, H123P, H123V, H123Y, Y123H, P124G, P124I, P124L, P124W, G125H, G125I, G125A, G125M, G125K, M126D, M126H, M126K, M126I, M126N, M1260, M126S, M126Y, N127H, N127S, N127D, N127K, N127R, H128R, R129H, R129Q, R129V, R129I, R129E, R129V, 11321, 1132F, T133V, T133E, T133G, T133K, E134A, E134E, E134G, E134I, G135G, G135V, 1136G, 1136L, 1136T, 1137A, 1137D, 1137E, L137M, 1137S, A138D, A138E, A138G, S138A, A138N, A138S, A138T, A138V, A138Y, D139E, D139I, D139C, D139L, D139M, E140A, E140C, E140L, E140R, A142N, A142D, A142G, A142A, A142L, A142S, A142T, A142N, A142S, A142V, A143D, A143E, A143G, A143D, A143G, A143E, A143L, A143W, A143M, A143S, A143Q, A143R, C146R, S146A, S146C, S146D, S146F, S146R, S146T, D147D, D147L, D147F, D147G, D147Y, Y147T, Y147R, Y147D, D147R, F148L, F148F, F148R, F148Y, F149C, F149M, F149R, F149Y, M151F, M151P, M151R, M151V, R152C, R152F, R152H, R152P, R152R, R153C, R153Q, R153R, R153V, Q154E, Q154H, Q154M, Q154R, Q154L, Q154S, Q154V, E155F, E155G, E155I, E155K, E155P, E155V, E155D, 1156A, 1156F, 1156D, 1156K, 1156N, 1156R, 1156Y, E157A, E157F, E1571, E157P, E157T, E157V, N157K, K157N, K157R, A158Q, A158K, A158V, Q159F, Q159K, Q159L, Q159N, K160A, K160S, K160E, K160K, K160N, K161I, K161A, K16IN, K161Q, K161S, K161T, A162D, A162Q, R162H, R162P, A162S, Q163G, Q163H, Q163N, Q163R, S164I, S164R, S164Y, S165A, S165D, S165I, S165T, S165Y, T166D, T166K, T166I, T166N, T166P, T166R, D167S and/or D167N mutation in a TadA reference sequence (e.g., TadA*7.10,ecTadA, or TadA8e), and any alternative mutation at the corresponding position, or any substitution from R26, W23, E27, H36, R47, P48, R51, H52, R74, 176, V82, V88, M94, 195, H96, A106, D108, A109, K110, T111, A114, D119, H122, H123, M126, N127, A142, S146, D147, F149, R152, Q154, E155, 1156, E157, K161, T166, and/or D167, with respect to a TadA reference sequence, or a substitution of 2-50 amino acids in a TadA reference sequence, which may be selected from W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, 1156F, K157N, K161N, T166I, and D167N, or one or more corresponding mutations in another adenosine deaminase. Additional mutations are described in U.S. Patent Application Publication No. 2022/0307003 A1 and International Patent Application Publications No. WO 2023/288304 A2 and WO 2023/034959 A2, the disclosures of which are incorporated herein by reference in their entirety for all purposes.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA (wt)) linked to a TadA*. Throughout the present disclosure, an adenosine deaminase base editor that comprises a single TadA* domain and a TadA (wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA (wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

In some embodiments, an APOBEC deaminase incorporated into a base editor can comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (TADC)” domains. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, 149K, 149M, 149N, 149Q, I49T, G67W, I76H, I76R, 176W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+149N; 1.17+E27G+149N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are
indicated with reference to TadA*8.20.

| | location in structure | | | | | | | | | | |
|---|---|---|---|---|---|---|---|---|---|---|---|
| | N/A | S h1 | S h1 | S h1 | NAS | NAS | NAS | NAS | | S | |
| | Amino Acid No. (*START Met is AA#1) | | | | | | | | | | |
| | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | W | | |
| 1.10 | | | | | C | | | | | I | |
| 1.11 | | | G | | Q | | | | | | |
| 1.12 | | | | A | H | | | M | | I | |
| 1.13 | | | | | | | | Q | | I | |
| 1.14 | H | | | | | | | K | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

“S” indicates “Surface,” and “NAS” indicates “Near Active Site.”

TABLE 6A
Adenosine Deaminase Variants. Mutations are indicated with reference to TadA*8.20.

| | location in structure | | | | | | | | | | | |
|---|---|---|---|---|---|---|---|---|---|---|---|---|
| | I | NAS | | NAS | S | | S | S | S | | S | |
| | Amino Acid No. (*START Met is AA#1) | | | | | | | | | | | |
| | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

“I” indicates “Internal,” “S” indicates “Surface,” and “NAS” indicates “Near Active Site.”

TABLE 6B
Adenosine deaminase variants. Mutations are
indicated with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| TadA*8.20 | E | P | V | I | S | F | R | G | G | A |
| Alterations | G/S/H | G/A/K | I/L/F | K | T | L/A | C | H | M | E |
| Evaluated | | | | | | | | | | |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| TadA*8.20 | E | P | V | I | S | F | R | G | G | A |
| Alterations | G/S/H | G/A/K | I/L/F | K | T | L/A | C | H | M | E |
| Evaluated | | | | | | | | | | |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| TadA*8.20 | E | P | V | I | S | F | R | G | G | A |
| Alterations | G/S/H | G/A/K | I/L/F | K | T | L/A | C | H | M | E |
| Evaluated | | | | | | | | | | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| TadA*8.20 | E | P | V | I | S | F | R | G | G | A |
| Alterations | G/S/H | G/A/K | I/L/F | K | T | L/A | C | H | M | E |
| Evaluated | | | | | | | | | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| TadA*8.20 | E | P | V | I | S | F | R | G | G | A |
| Alterations | G/S/H | G/A/K | I/L/F | K | T | L/A | C | H | M | E |
| Evaluated | | | | | | | | | | |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| TadA*8.20 | E | P | V | I | S | F | R | G | G | A |
| Alterations | G/S/H | G/A/K | I/L/F | K | T | L/A | C | H | M | E |
| Evaluated | | | | | | | | | | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminase variants. Mutations are indicated with reference to variant 1.2 (Table 6A).

| | Alternative Variant | Residue identity (START Met is amino acid #1) | | | | | | | | |
|---|---|---|---|---|---|---|---|---|---|---|
| Variant Name | Names | 4 | 6 | 17 | 23 | 76 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| Reference | 1.2 (see Table 6A) | V | F | T | R | I | D | G | T | A |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |

| | Alternative Variant | Residue identity (START Met is amino acid #1) | | | | | | | | |
|---|---|---|---|---|---|---|---|---|---|---|
| Variant Name | Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| Reference | 1.2 (see Table 6A) | D | H | N | A | R | A | Q | A | T |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to TadA*8.20.

| | AA Positions | | | | | | | | | | | | | | |
|---|---|---|---|---|---|---|---|---|---|---|---|---|---|---|---|
| | 6 | 27 | 49 | 76 | 77 | 82 | 107 | 112 | 114 | 115 | 119 | 122 | 127 | 142 | 143 |
| TadA*8.20 | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table | | | | | | | | | | | | | | | |
| 6C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| TadA*8.20 | F | E | I | Y | D | S | R | G | A | G | D | H | N | A | A |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to TadA*8.19/8.20.

| | AA positions: | | | | | | | | | | | | |
|---|---|---|---|---|---|---|---|---|---|---|---|---|---|
| | 17 | 27 | 48 | 49 | 76 | 82 | 84 | 118 | 142 | 147 | 149 | 166 | 167 |
| ABE8.19m/8.20m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 1010. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite-2 parts, while monopartite NLSs are not). The NLS of nucleoplasmin, KR[PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows: PKKKRKVEGADKRTADGSEFESPKKKRKV (SEQ ID NO: 328).

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein, for example ABE Variant A, ABE Variant B, ABE Variant C, ABE Variant D, ABE Variant E, ABE Variant F, ABE Variant G, ABE Variant H, ABE Variant I, ABE Variant J, ABE Variant K, or ABE Variant D comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some embodiments, cellular DNA repair response to the presence of U:G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fe domain, a fibritin foldon domain, an FK506 binding protein (FKBP) domain, an FKBP binding domain (FRB) domain of mTOR, a foldon domain, a fragment X domain, a GAI domain, a GIDI domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voβ, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS) n (SEQ ID NO: 247), and (G) n to more rigid linkers of the form (EAAAK) n (SEQ ID NO: 248), (SGGS) n (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32 (6): 577-82; the entire contents are incorporated herein by reference) and (XP) n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS) n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGSP
TSTEEGTSTEPSEGSAPGTSTEPSEGSAPGTSESATPESGPGSEPATSGG
SGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSGGS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P (AP) 4 (SEQ ID NO: 367), P (AP) 7 (SEQ ID NO: 368), P (AP) 10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10 (1): 439; the entire contents are incorporated herein by reference). Such proline-rich linkers are also termed “rigid” linkers.

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations: unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3: eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### CD117-Binding Polypeptides

Generation and Screening of Antibodies that Bind to a wild type CD117 Polypeptide or Peptide

Antibodies, including recombinantly produced antibodies, that specifically bind to wild type CD117 but show reduced binding to a CD117 variant polypeptide or peptide thereof (e.g., a variant produced by the methods provided herein) are provided and described herein. In embodiments, the antibodies are ABTx025, ABTx030, ABTx052, ABTx061, ABTx062, ABTx070, ABTx071, ABTx196, ABTx198, ABTx202, ABTx203, ABTx205, ABTx206, ABTx248, ABTx250, ABTx251, ABTx253, ABTx254, ABTx255, ABTx256, ABTx265, ABTx268, ABTx270, ABTx271, ABTx272, ABTx273, ABTx274, ABTx307, ABTx308, ABTx309, and ABTx313, or antigen binding portions thereof, as described herein.

Methods for generating antibodies against a protein or peptide of interest are known and practiced in the art. When animals are immunized with antigens they respond by generating a polyclonal antibody response comprised of many individual monoclonal antibody specificities. It is the sum of these individual specificities that make polyclonal antibodies useful in so many different assays. Individual monoclonal antibodies were originally isolated by immortalizing individual B cells using hybridoma technology (Kohler and Milstein, Nature 256, 495, 2011), in which B cells from an immunized animal are fused with a myeloma cell. With the advent of molecular biology, in vitro methods to generate antibodies against proteins of interest, such as wild type CD117, have been developed.

The terms “antigen of interest” or “target protein” are used herein interchangeably and refer generally to the agent recognized and specifically bound by an antibody. In an embodiment, such an antigen of interest or target protein is the wild type CD117 polypeptide, or an antigenic and/or immunogenic portion thereof.

An antibody is a polypeptide chain-containing molecular structure with a specific shape that specifically binds an epitope, where one or more non-covalent binding interactions stabilize the complex between the molecular structure and the epitope. In one embodiment, an antibody molecule is an immunoglobulin (e.g., IgG, IgM, IgA, IgE, IgD). Antibodies from a variety of sources, e.g., human, rodent, rabbit, cow, sheep, pig, dog, or fowl are considered “antibodies.” Numerous antibody coding sequences have been described; and others may be raised by methods well-known in the art.

For example, antibodies or antigen binding fragments may be produced by genetic engineering. Antibody coding sequences of interest include those encoded by native sequences, as well as nucleic acids that, by virtue of the degeneracy of the genetic code, are not identical in sequence to a wild type nucleic acid sequence. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues). Variants also include fragments of the polypeptides disclosed herein, particularly biologically active fragments and/or fragments corresponding to functional domains. Techniques for in vitro mutagenesis of cloned genes are known. Also included in some aspects and embodiments herein are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions obtained from antibody producing cells of one species with the constant light and heavy chain regions from another. Typically, chimeric antibodies utilize rodent or rabbit variable regions and human constant regions, in order to produce an antibody with predominantly human domains. The production of such chimeric antibodies is well known in the art and may be achieved by standard means (as described, e.g., in U.S. Pat. No. 5,624,659, incorporated fully herein by reference).

Humanized antibodies are engineered to contain even more human-like immunoglobulin domains and incorporate only the complementarity-determining regions of the animal-derived antibody. This is accomplished by carefully examining the sequence of the hyper-variable loops of the variable regions of the monoclonal antibody and fitting them to the structure of the human antibody chains. Although apparently complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)2, or other fragments) may be synthesized. “Fragment,” or minimal immunoglobulins may be designed utilizing recombinant immunoglobulin techniques. For instance, “Fv” immunoglobulins for use in some aspects and embodiments herein may be produced by synthesizing a variable light chain region and a variable heavy chain region. Combinations of antibodies are also of interest, e.g., diabodies, which comprise two distinct Fv specificities.

Immunoglobulins may be modified post-translationally, e.g., to add chemical linkers, detectable moieties, such as fluorescent dyes, enzymes, substrates, chemiluminescent moieties and the like, or specific binding moieties, such as streptavidin, avidin, or biotin, and the like may be utilized in the methods and compositions of some aspects and embodiments herein.

### Screening of Libraries for CD117-Binding Polypeptides or Peptides

Methods for high throughput screening of polypeptide (e.g., antibody or antigen-binding antibody fragment) libraries for molecules capable of binding to the wild type CD117 polypeptide or peptide (and/or epitopes within the CD117 polypeptide or peptide) include, without limitation, display techniques including phage display, bacterial display, yeast display, mammalian display, ribosome display, mRNA display, and cDNA display. The use of phage display to isolate ligands that bind biologically relevant molecules has been reviewed, e.g., in Felici et al. (Biotechnol. Annual Rev. 1:149-183, 1995), Katz (Annual Rev. Biophys. Biomol. Struct. 26:27-45, 1997), and Hoogenboom et al. (Immunotechnology 4:1-20, 1998). Several randomized combinatorial peptide libraries have been constructed to select for polypeptides that bind different targets, e.g., cell surface receptors or DNA (reviewed by Kay (Perspect. Drug Discovery Des. 2, 251-268, 1995), Kay et al., (Mol. Divers. 1:139-140, 1996)). Proteins and multimeric proteins have been successfully phage-displayed as functional molecules (see. e.g., EP 0349578A, EP 4527839A, EP 0589877A; Chiswell and McCafferty (Trends Biotechnol. 10, 80-84 1992)). In addition, functional antibody fragments (e.g., Fab, single-chain Fv [scFv]) have been expressed as reported by McCafferty et al. (Nature 348:552-554, 1990), Barbas et al. (Proc. Natl. Acad Sci. USA 88:7978-7982, 1991), and Clackson et al. (Nature 352:624-628, 1991). These references are hereby incorporated by reference in their entirety.

In addition to generating CD117 binding polypeptides (e.g., CD117 binding polypeptides, antibodies, and antigen-binding fragments thereof) of some aspects and embodiments herein, in vitro display techniques, which are known and practiced in the art, also provide methods for improving the affinity of an anti-CD117 variant-binding polypeptide, antibody, or antigen-binding fragments thereof. For instance, rather than screening libraries of antibodies and fragments thereof containing completely randomized hypervariable regions, narrower libraries of antibodies and antigen-binding fragments thereof that feature targeted mutations at specific sites within hypervariable regions can be screened. This can be accomplished, for example, by assembling libraries of polynucleotides encoding antibodies or antigen-binding fragments thereof that encode random mutations only at particular sites within hypervariable regions. These polynucleotides can then be expressed in, e.g., filamentous phage, bacterial cells, yeast cells, mammalian cells, or in vitro using, e.g., ribosome display, mRNA display, or cDNA display techniques in order to screen for antibodies or antigen-binding fragments thereof that specifically bind to the wild type CD117 polypeptide or peptide (and epitopes thereof) with improved binding affinity. Yeast display, for instance, is well-suited for affinity maturation, and has been used previously to improve the affinity of a single-chain antibody to a K_{D} of 48 fM (Boder et al. (Proc Natl Acad Sci USA 97:10701, 2000)).

Additional in vitro techniques that can be used for the generation and affinity maturation of CD117-binding polypeptides, antibodies, and antigen-binding fragments thereof (e.g., single-chain polypeptides, antibodies, and antigen-binding fragments thereof) of some aspects and embodiments herein include the screening of combinatorial libraries of antibodies or antigen-binding fragments thereof for functional molecules capable of specifically binding to peptides derived from the CD117 polypeptide. Combinatorial antibody libraries can be obtained, e.g., by expression of polynucleotides encoding randomized hypervariable regions of an antibody or antigen-binding fragment thereof in a eukaryotic or prokaryotic cell. This can be achieved, e.g., using gene expression techniques described herein or known in the art. Heterogeneous mixtures of antibodies can be purified, e.g., by Protein A or Protein G selection, sizing column chromatography), centrifugation, differential solubility, and/or by any other standard technique for the purification of proteins. Libraries of combinatorial libraries thus obtained can be screened, e.g., by incubating a heterogeneous mixture of these antibodies with a peptide derived from the CD117 polypeptide that has been immobilized to a surface for a period of time sufficient to allow antibody-antigen binding. Non-binding antibodies or fragments thereof can be removed by washing the surface with an appropriate buffer (e.g., a solution buffered at physiological pH (approximately 7.4) and containing physiological salt concentrations and ionic strength, and optionally containing a detergent, such as TWEEN-20®). Antibodies that remain bound can subsequently be detected, e.g., using an ELISA-based detection protocol (see, e.g., U.S. Pat. No. 4,661,445; incorporated herein by reference).

Additional techniques for screening combinatorial libraries of polypeptides (e.g., antibodies, and antigen-binding fragments thereof) for those that specifically bind to CD117 polypeptide-derived peptides include the screening of one-bead-one-compound libraries of antibody fragments. Antibody fragments can be chemically synthesized on a solid bead (e.g., using established split-and-pool solid phase peptide synthesis protocols) composed of a hydrophilic, water-swellable material such that each bead displays a single antibody fragment. Heterogeneous bead mixtures can then be incubated with a CD117 polypeptide-derived peptide that is optionally labeled with a detectable moiety (e.g., a fluorescent dye) or that is conjugated to an epitope tag (e.g., biotin, avidin, FLAG tag, HA tag) that can later be detected by treatment with a complementary tag (e.g., avidin, biotin, anti-FLAG antibody, anti-HA antibody, respectively). Beads containing antibody portions or fragments that specifically bind to CD117 polypeptide-derived peptide can be identified by analyzing the fluorescent properties of the beads following incubation with a fluorescently-labeled antigen or complementary tag (e.g., by confocal fluorescent microscopy or by fluorescence-activated bead sorting; see, e.g., Muller et al. (J. Biol. Chem., 16500-16505, 1996); incorporated herein by reference). Beads containing antibody fragments that specifically bind to CD117 polypeptide-derived peptides can thus be separated from those that do not contain high-affinity antibody fragments. The sequence of an antibody fragment that specifically binds to a CD117 polypeptide-derived peptide can be determined by techniques known in the art, including, e.g., Edman degradation, tandem mass spectrometry, matrix-assisted laser-desorption time-of-flight mass spectrometry (MALDI-TOF MS), nuclear magnetic resonance (NMR), and 2D gel electrophoresis, among others (see, e.g., WO 2004/062553; incorporated herein by reference).

### Methods of Identifying Antibodies and Ligands

Methods for high throughput screening of antibody, antibody fragment, and ligand libraries for molecules capable of binding the CD117 polypeptide or peptide can be used to identify antibodies suitable for the uses as described herein. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind to the CD117 polypeptide or peptide.

### Host Cells for Expression of Antibodies

Mammalian cells can be co-transfected with polynucleotides encoding the antibodies of some aspects and embodiments herein, which are expressed as recombinant polypeptides, and assembled into anti-CD117 antibodies by the host cell. In one embodiment, a mammalian cell is co-transfected with polynucleotides encoding the heavy and light chains of an anti-CD117 polypeptide antibody, which are expressed in the cell and assembled as the anti-CD117 antibody.

It is possible to express antibodies or antigen-binding fragments thereof in either prokaryotic or eukaryotic host cells. In certain embodiments, expression of polypeptides or antigen-binding fragments thereof is performed in eukaryotic cells, e.g., mammalian host cells, for optimal secretion of a properly folded and immunologically active antibody. Exemplary, nonlimiting mammalian host cells for expressing the recombinant antibodies or antigen-binding fragments thereof of some aspects and embodiments herein include Chinese Hamster Ovary (CHO cells) (including DHFR CHO cells, described in Urlaub and Chasin (1980, Proc. Natl. Acad. Sci. USA 77:4216-4220), used with a DHFR selectable marker, e.g., as described in Kaufman and Sharp (1982, Mol. Biol. 159:601-621), NSO myeloma cells, COS cells, HEK293T cells, SP2/0, NIH3T3, and BaF3 cells. Additional, nonlimiting cell types that may be useful for the expression of antibodies and fragments thereof include bacterial cells, such as BL-21 (DE3) E. coli cells, which can be transformed with vectors containing foreign DNA according to established protocols. Additional eukaryotic cells that may be useful for expression of antibodies include yeast cells, such as auxotrophic strains of S. cerevisiae, which can be transformed and selectively grown in incomplete medium according to established procedures known in the art. When recombinant expression vectors encoding antibody genes are introduced into mammalian host cells, the antibodies are produced by culturing the host cells for a period of time sufficient to allow for expression of the antibody protein in the host cells or secretion of the antibody into the culture medium in which the host cells are grown.

Polypeptides (e.g., antibodies or antigen-binding fragments thereof) can be recovered from the culture medium using standard protein purification methods. Host cells can also be used to produce portions of intact antibodies, such as Fab fragments or scFv molecules. Also included in some aspects and embodiments herein are methods in which the above procedure is varied according to established protocols known in the art. For example, it may be desirable to transfect a host cell with DNA encoding either the light chain or the heavy chain (but not both) of an anti-CD117 antibody of some aspects and embodiments herein in order to produce an antigen-binding fragment of the antibody.

Once a CD117-binding polypeptide (e.g., an anti-CD117 polypeptide antibody or an antigen-binding fragment thereof) of some aspects and embodiments herein has been produced by recombinant expression, it can be purified by any method known in the art, such as a method useful for purification of an immunoglobulin molecule, for example, by chromatography (e.g., ion exchange, affinity, affinity for antigen (e.g., a CD117 polypeptide or peptide) after Protein A or Protein G selection, and sizing column chromatography), centrifugation, differential solubility, or by any other standard technique for the purification of proteins. Further, a CD117 polypeptide (e.g., an anti-CD117 antibody of some aspects and embodiments described herein) or an antigen-binding portion or fragment thereof, can be fused to heterologous polypeptide sequences as known in the art, for example, to facilitate purification, e.g., a histidine tag, a detectable/detectably labeled marker, and the like.

Once isolated, an anti-CD117 antibody, or antigen-binding portion or fragment thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a Superdex. TM. 75 column (Pharmacia Biotech AB, Uppsala, Sweden).

### Mapping Epitopes of the CD117 Polypeptide

Anti-CD117 antibodies, and antigen-binding fragments thereof, can be produced by screening libraries of polypeptides (e.g., antibodies and antigen-binding fragments thereof) for functional molecules that are capable of binding to a wild type CD117 polypeptide or peptide that selectively bind to the wild type CD117 polypeptide or peptide compared with CD117 polypeptide variant polypeptides or peptides containing alterations, such as those provided herein. Epitopes can be modeled by screening antibodies or antigen-binding fragments thereof against a series of linear or cyclic peptides containing residues that correspond to a desired epitope within a CD117 polypeptide or peptide.

As an example, peptides containing individual fragments isolated from the CD117 polypeptide or peptide can be synthesized by peptide synthesis techniques known in the art. These peptides can be immobilized on a solid surface and screened for molecules that bind to anti-CD117 antibodies and antigen-binding fragments thereof, such as representative antibodies ABTx025, ABTx030, ABTx052, ABTx061, ABTx062, ABTx070, and ABTx071, or antigen binding portions thereof, as described herein, e.g., using an ELISA-based screening platform using established procedures. Using this assay, peptides that specifically bind to the anti-CD117 antibodies with high affinity therefore contain residues within epitopes of the CD117 polypeptide antigen that preferentially bind these antibodies. Peptides identified in this manner can be used to screen libraries of antibodies and antigen-binding fragments thereof in order to identify anti-CD117 antibodies useful in generating anti-CD117 antibodies of some aspects and embodiments herein.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas9 nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient HDR with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (IssDNA) donors.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138 (7): 2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 974-1009. Inteins suitable for use in embodiments of the present disclosure and methods for use thereof are described in U.S. Pat. No. 10,526,401, International Patent Application Publication No. WO 2013/045632, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, referenced to SEQ ID NO: 197.

### Pharmaceutical Compositions

In some aspects, the present disclosure provides a pharmaceutical composition comprising any of the cells, polynucleotides, polypeptides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., bone marrow). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Kits

The disclosure provides kits for the treatment of a hemoglobinopathy (e.g., sickle cell disease) in a subject according to the methods provided herein. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is a Cas9 or Cas 12 polypeptide or variant thereof. In some embodiments, the polynucleotide encoding the base editor is a mRNA sequence. In some embodiments, the deaminase is an adenosine deaminase. In some embodiments, the kit comprises an edited cell and instructions regarding the use of such cell. In some instances, the kit comprises an anti-CD117 antibody (e.g., ABTx025, ABTx030, ABTx052, ABTx061, ABTx062, ABTx070, ABTx071, ABTx196, ABTx198, ABTx202, ABTx203, ABTx205, ABTx206, ABTx248, ABTx250, ABTx251, ABTx253, ABTx254, ABTx255, ABTx256, ABTx265, ABTx268, ABTx270, ABTx271, ABTx272, ABTx273, ABTx274, ABTx307, ABTx308, ABTx309, and/or ABTx313) suitable for use in the methods provided herein.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Development of an Engineered Stem Cell Antibody-Paired Evasion (ESCAPE) Approach for Engraftment of Hematopoietic Stem Cells (HSCs) Engineered to Overexpress Fetal Hemoglobin

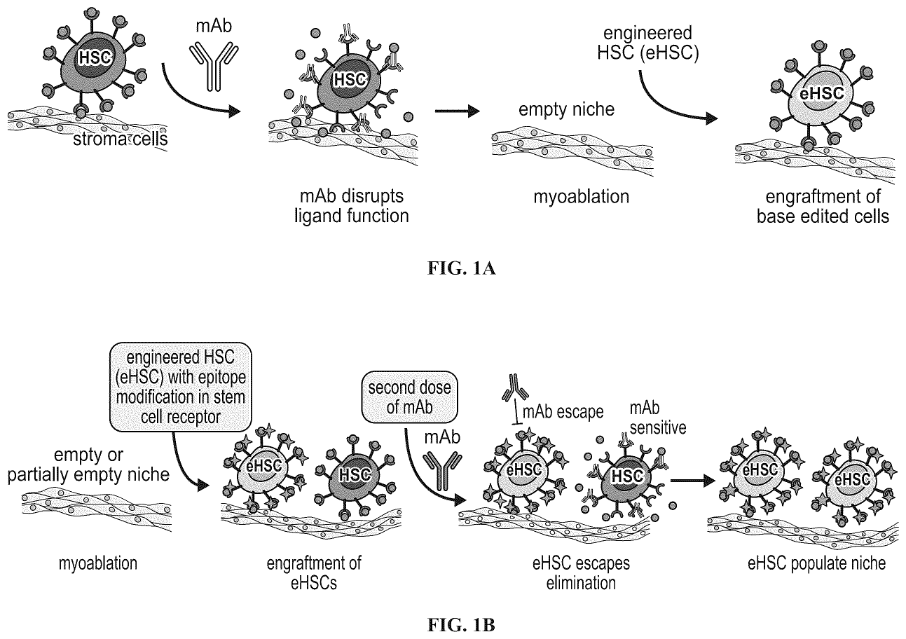

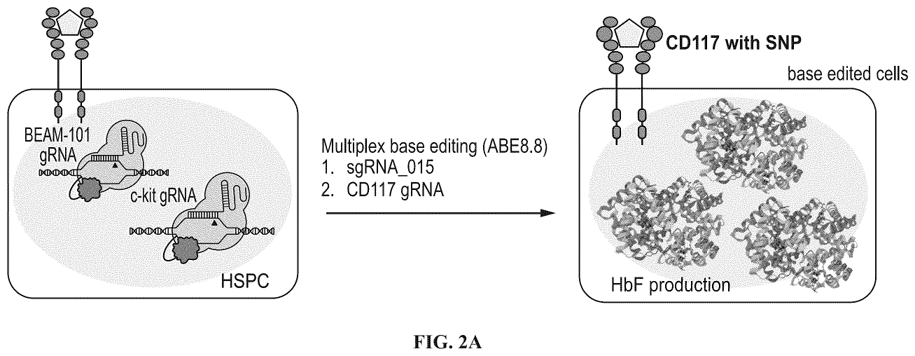

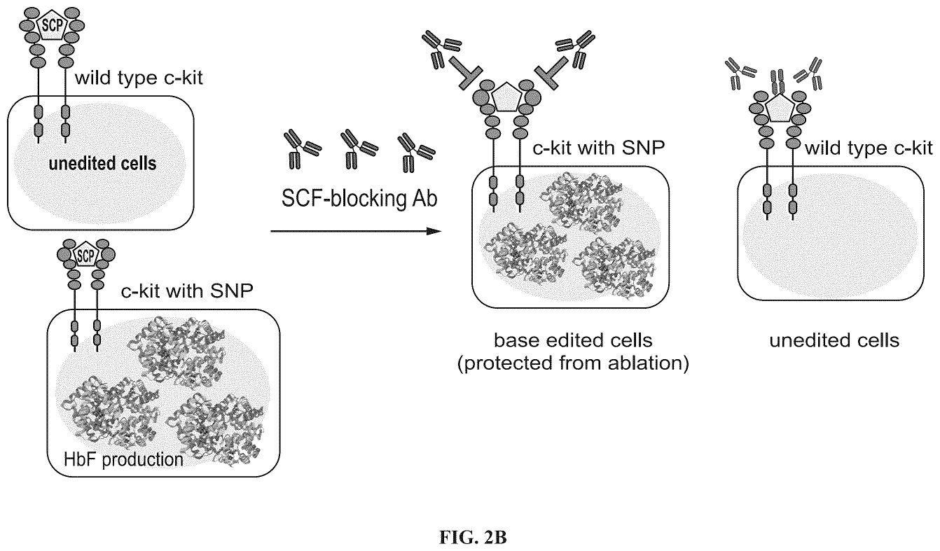

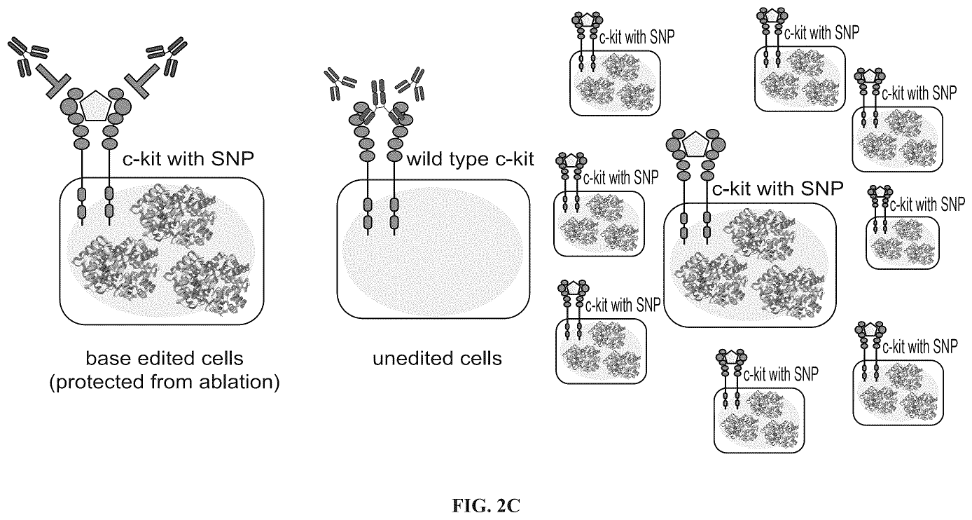

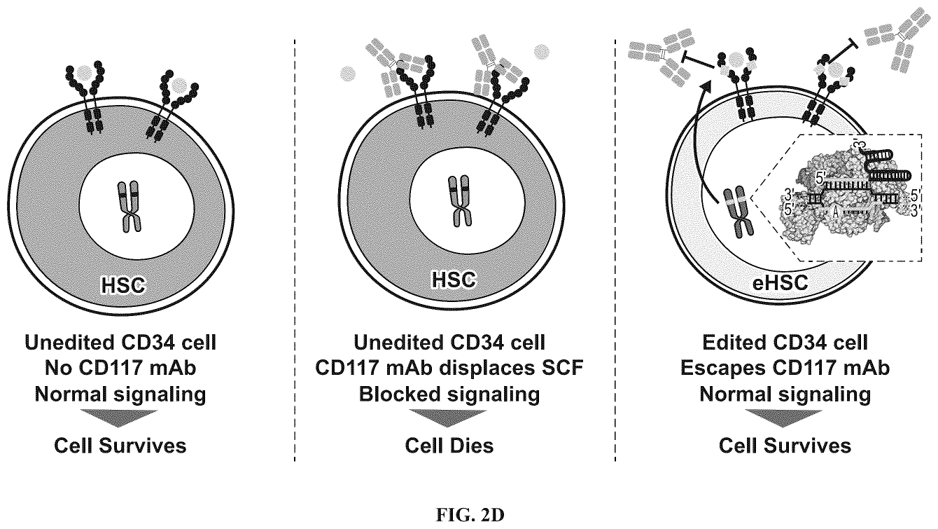

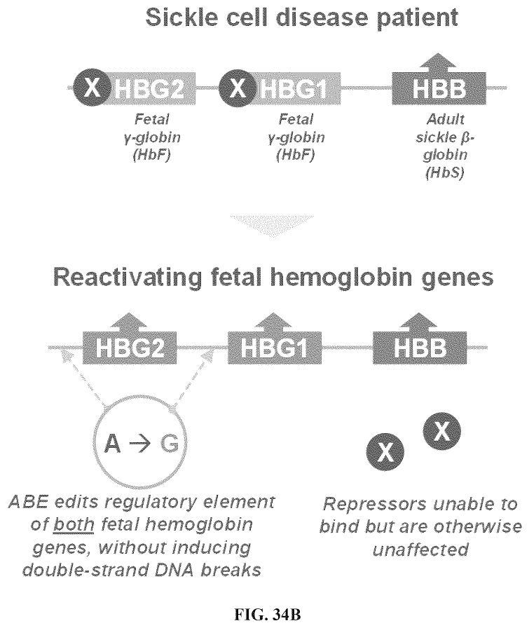

Experiments were undertaken to leverage base editing technologies to produce engineered hemopoietic stem cells (eHSCs) that could be coupled with a monoclonal antibody (mAb) strategy capable of clearing the niche of a patient's bone marrow to levels sufficient for eHSC engraftment and preserving eHSC viability in the presence of the conditioning mAb. Using base editing technology (see, e.g., FIGS. 34A and 34B), methods were developed to enhance the flexibility and potency of a non-genotoxic conditioning approach (e.g., use of monoclonal antibodies) through intentional, non-synonymous mutations within an epitope of an essential protein expressed on the surface of long-term HSCs (e.g., CD117). This essential, cell-surface variant protein was designed to selectively escape mAb-induced ablation. Contrastingly, unedited, or endogenous HSCs expressing wild type CD117 remained sensitive to mAb interference and were destroyed (FIGS. 1A, 1B, 2A-2D, and 12). Because the CD117 epitope-engineered HSCs were designed for transplant and to be resistant to a conditioning reagent, high concentrations of mAb can be used to clear the niche for engraftment, long half-lives of administered mAb conditioning reagent can be tolerated, and additional doses of mAb can be administered if required. The programmed epitope edits were combined with base editing strategies to upregulate fetal-hemoglobin production in erythroid cells, a mechanism that can ameliorate the effects of sickle cell disease.

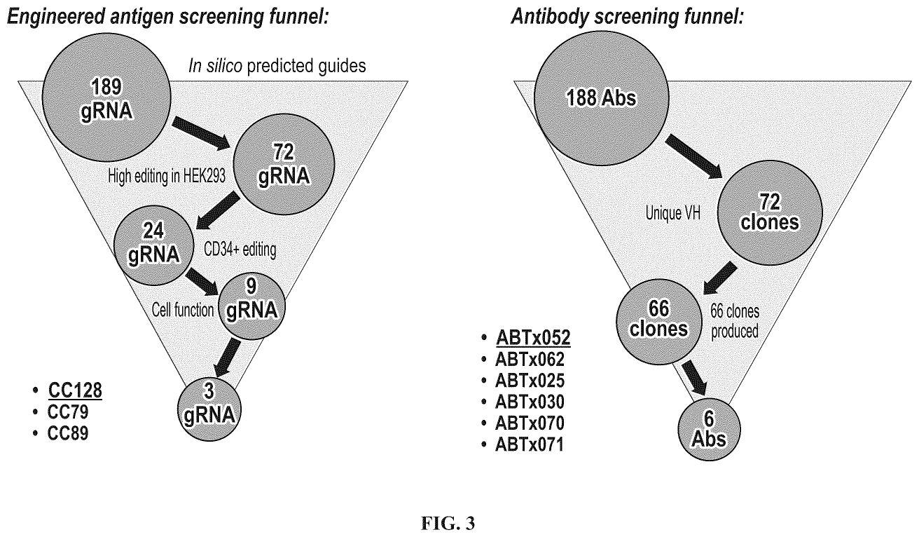

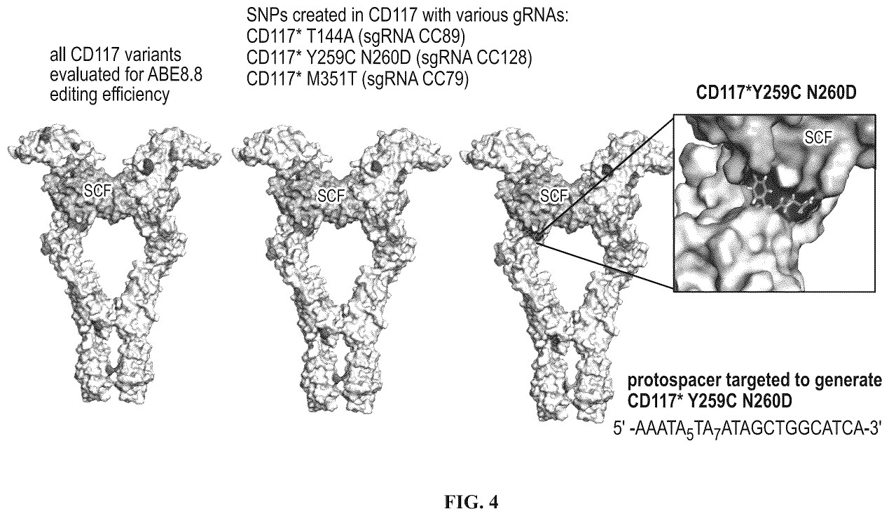

An experiment was undertaken to screen 189 guide RNAs predicted in silico to be suitable for use with ABE8.8 for introducing an alteration to a CD117 polynucleotide (FIG. 3). 72 of the guides demonstrated high editing in HEK293 T cells. Of those 72 guides, 24 demonstrated editing in CD34+ cells. Of those 24 guides, 3 (i.e., CC128, CC79, and CC89) were identified as optimal for altering a CD117 polynucleotide sequence. CD117 polypeptide alterations corresponding to each of the three guides are shown in FIG. 4. The alteration T144A corresponded to sgRNA CC89, the alterations Y259C and N260D corresponded to sgRNA CC128, and the alteration M351T corresponded to sgRNA CC79. The protospacer corresponding to sgRNA CC128 was the following: 5′-AAATA₅TA₇ATAGCTGGCATCA-3′ (SEQ ID NO: 830), where the subscripts indicate the A5 and A7 nucleotides that were the targets for base editing to yield the alterations 5G and 7G, respectively, which corresponded to the CD117 amino acid alterations Y259C and N260D (FIG. 4). The amino acids Y259 and N260 of CD117 are near the binding site for stem cell factor (SCF). Table 8A below provides a summary of representative CD117 variants prepared using guides identified in the screen including the desired/target CD117 variant outcomes and the observed protein variants at maximum editing. These were selected based on favorable editing outcomes and no clear biological phenotype. Table 8A also lists observed editing rates when the cells were multiplex edited using a CD117-targeting guide in combination with a guide targeting the promoter region of HBG1/2 (sgRNA_015). Four of the guides listed in Table 8A had homology to a cynomolgus (cyno) CD117 gene. None of the guides listed in Table 8A (excepting the control knock-out (KP) guide) had an erythroid phenotype, a myeloid phenotype, or a CD117 phosphorylation phenotype. Cells edited using the guides listed in Table 8A all showed an in vivo phenotype showing major differences from control cohort in editing or human chimerism at 8 weeks. Table 8A shows that human chimerism and multi-lineage hematopoietic reconstitution potential and retention of editing at 8 was observed at levels similar to unedited controls.

TABLE 8A
Summary of ABE8.8-compatible engineered CD117 variant biology.¹

| | CD117 | | | | |
|---|---|---|---|---|---|
| | variant | | Observed protein | | |
| Variant ID | desired | Domain/Epitope | variants at max | Erythroid | Myeloid |
| (comment) | outcome | Group | editing | phenotype | phenotype |
| CC90 | E81G | other | 82% E81G | N | N |
| CC89 | T144A | other | 73% T144A; 4% | N | N |
| | | | T144A + N145D | | |
| CC119 | D45G | other | 88% D45G | N | N |
| CC128 (SCF | Y259C + | funct impt/SCF | 89% Y259C + N260D; | N | N |
| blocker) | N260D | binding | 5% N260D | | |
| CC84 | Q79R | other | 79% Q79R; 6% | N | N |
| | | | Q79R + N80D | | |
| CC79 | M351T | dimerization | 77% M351T; 5% | N | N |
| | | | M315T + Y350H | | |
| KO (splice | | | 7G edit | Y | N |
| site | | | | | |
| disruption) | | | | | |

| | CD117 | Mutliplexed | 8-week | | Low to |
|---|---|---|---|---|---|
| Variant ID | phosphorylation | with HBG1/2 - | in vivo | Cyno | no Ab |
| (comment) | phenotype | 114 sgRNA* | phenotype** | homology | binders |
| CC90 | N | ++ | N | Y | 1 |
| CC89 | N | + | N | N | 4 |
| CC119 | N | +++ | N | N | 2 |
| CC128 (SCF | N | +++ | N | Y | 4 |
| blocker) | | | | | |
| CC84 | N | + | N | Y | 2 |
| CC79 | N | +++ | N | Y | 3 |
| KO (splice | Y | N/A | Y | N/A | N/A |
| site | | | | | |
| disruption) | | | | | |

¹*For multiplexing, value is level of HBG1/2 editing that can be achieved with CD117 editing: + <40%; ++ 40-60%; +++ >60%
**in vivo phenotype in terms of major differences from control cohort in editing or human chimerism at 8 weeks

In addition to guide RNA screen, 188 human antibodies identified from a CD117 immune phage library were screened to identify six antibodies (ABTx052, ABTx062, ABTx025, ABTx030, ABTx070, and ABTx071) that bound wild type CD117 but had reduced binding to CD117 polypeptides altered using the guides identified in the guide RNA screen (e.g., CC128, CC79, and CC89) (FIG. 3). Table 8B provides a summary of the observed binding, or lack thereof, between the listed antibodies and the indicated CD117 variants prepared by editing M07e cells using the indicated guides (i.e., CC79, CC89, and CC128). M07e cells are not dependent on SCF for growth, but CD117 can be activated upon SCF stimulation. All of the antibodies listed in Table 8B bound similarly to human and cyno CD117. Cynomolgus monkey is a suitable species for investigative new drug-enabling toxicology studies. ABTx052, ABTx061, ABTx062 and ABTx025 competed with SCF to bind CD117.

TABLE 8B
CD117 antibody/edit pairs identified.

| | | | | | | | KD(M) | KD(M) |
|---|---|---|---|---|---|---|---|---|
| | | CC79- | CC89- | CC128- | SCF | ABTx052 | Human | Cyno |
| Ab ID | M07e | M07e | M07e | M07e | blocking | blocking | CD117 | CD117 |
| ABTx061 | binding | binding | binding | no | Yes | Yes | 1.86E−08 | 2.95E−08 |
| | | | | binding | | | | |
| ABTx062 | binding | binding | binding | no | Yes | Yes | 1.80E−08 | 3.18E−08 |
| | | | | binding | | | | |
| ABTx070 | binding | binding | no | binding | No | No | 6.50E−09 | 4.86E−10 |
| | | | binding | | | | | |
| ABTx071 | binding | binding | no | binding | No | No | 5.23E−10 | 4.28E−09 |
| | | | binding | | | | | |
| ABTx025 | binding | binding | binding | no | Yes | Yes | 1.25E−09 | 5.31E−09 |
| | | | | binding | | | | |
| ABTx030 | binding | low | binding | binding | No | No | 4.49E−10 | 8.30E−10 |
| | | binding | | | | | | |
| ABTx052 | binding | binding | binding | low/no | Yes | Yes | <1.0E−12 | <1.0E−12 |
| | | | | binding | | | | |

“Cyno” is an abbreviation for “cynomolgus.”

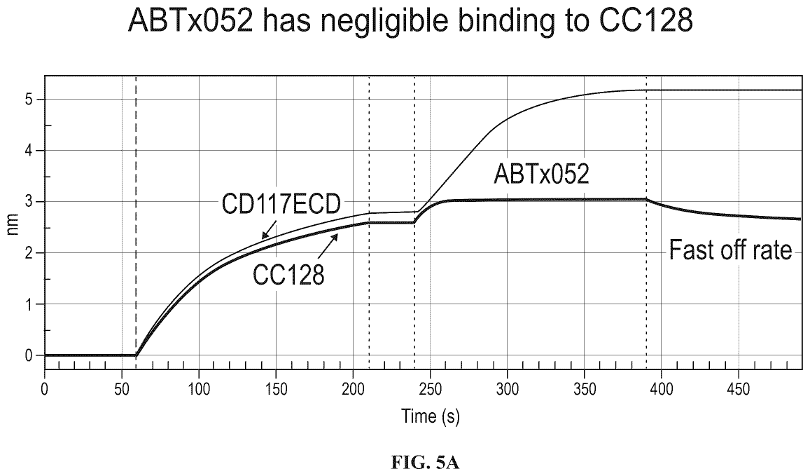

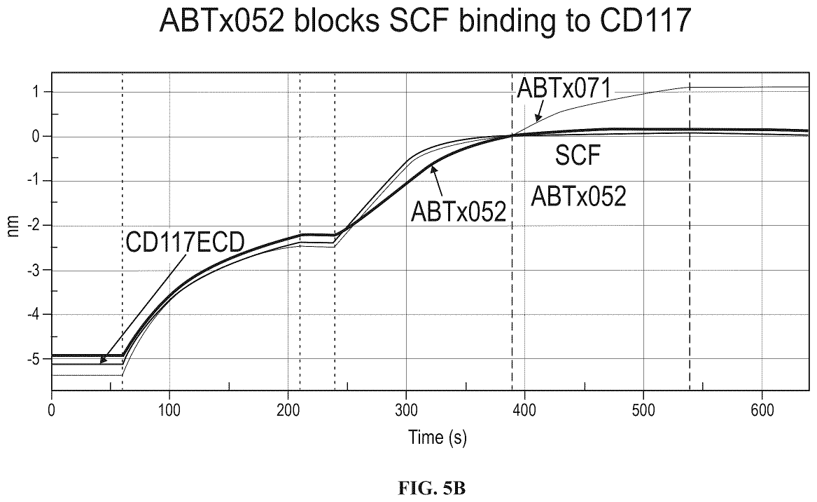

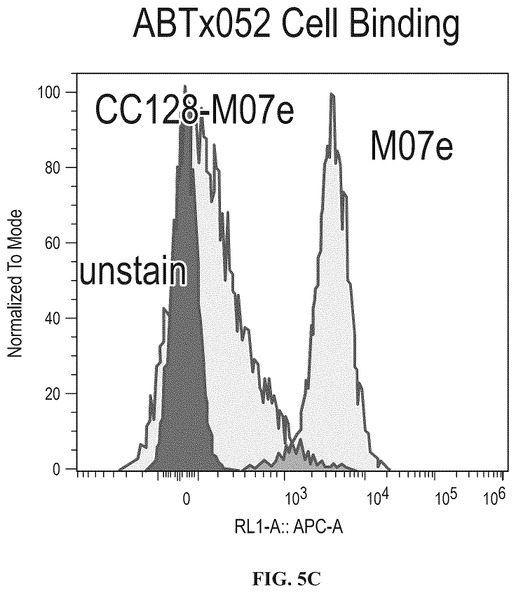

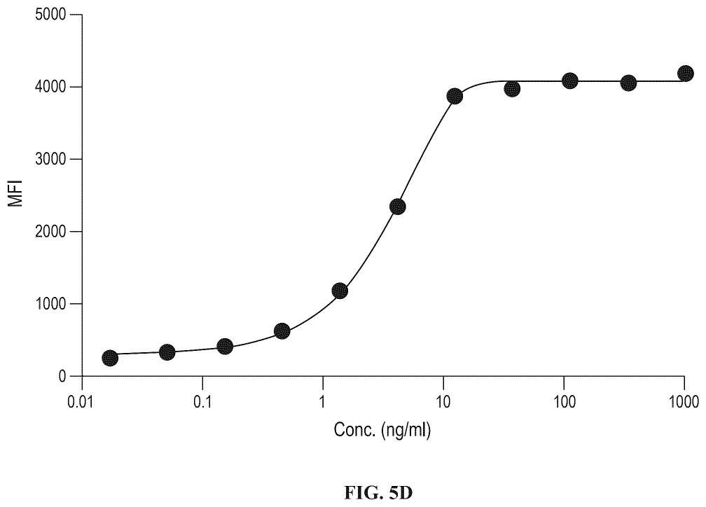

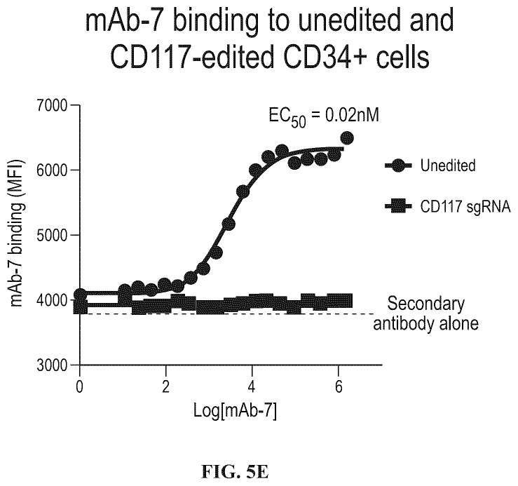

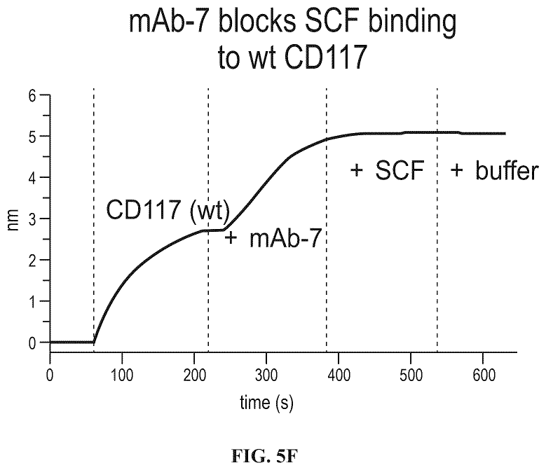

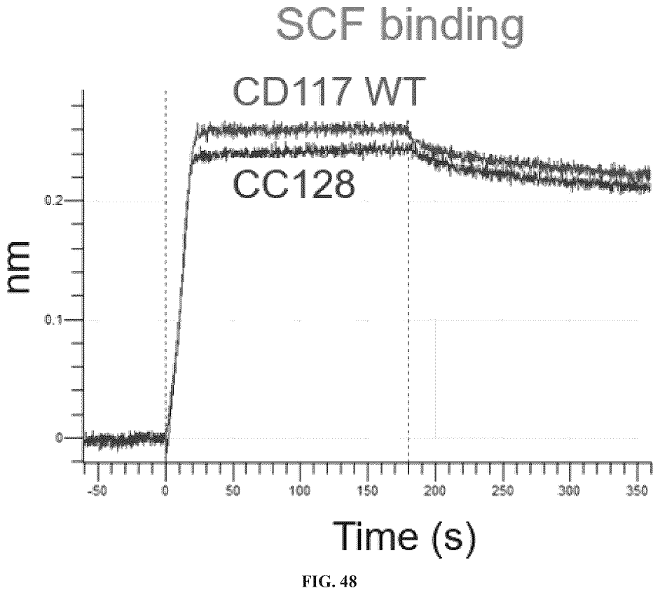

Experiments were undertaken to characterizing the binding of stem cell factor (SCF) and ABTx052 to CD117 polypeptides altered using the CC128 guide in combination with ABE8.8 (FIGS. 5A-5F and 48). It was determined that ABTx052 had negligible binding to the CD117 polypeptides altered using the CC128 guide in combination with ABE8.8. It was also determined that ABTx052 blocked SCF binding to wild type CD117.

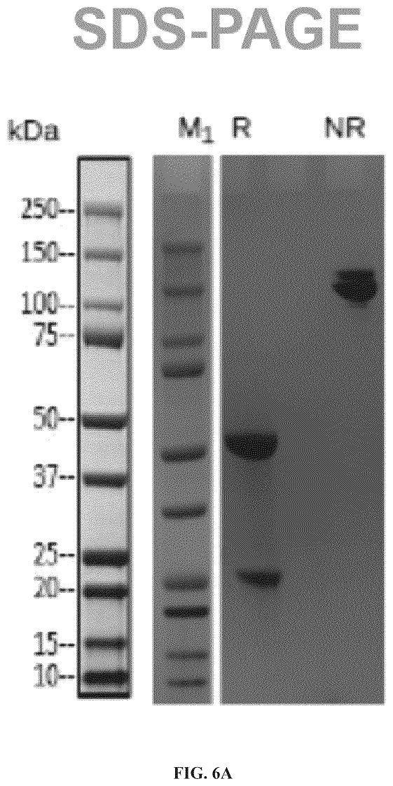

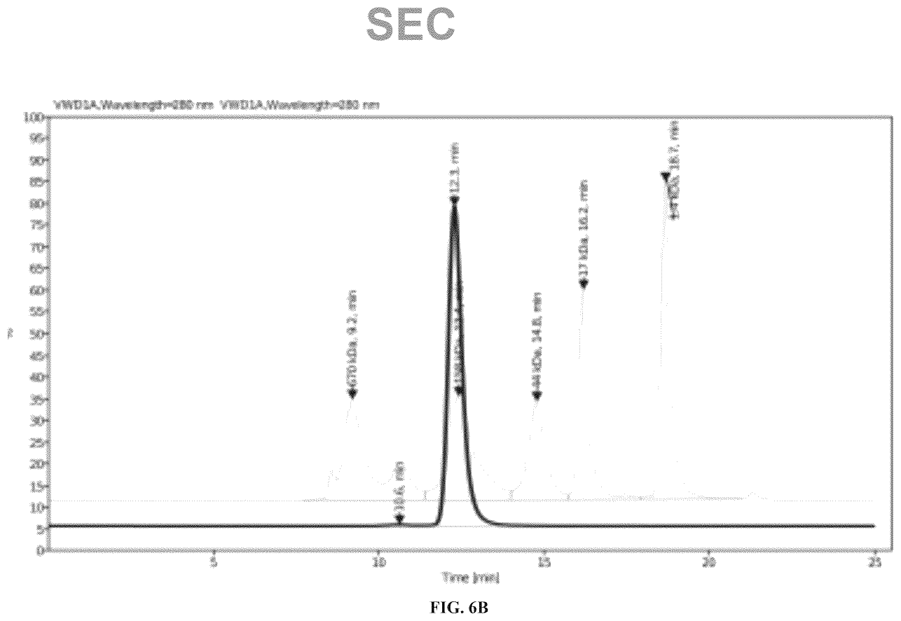

As a quality control measure, experiments were undertaken to characterize the ABTx052 antibody (FIGS. 6A and 6B) using SDS-PAGE and size-exclusion chromatography (SEC). The results of the characterization are provided in Table 9 below.

TABLE 9
Summary of ABTx052 properties.

| Assay | Result |
|---|---|
| Purity (SDS-PAGE) | >95% |
| % monomer (SEC) | >99% |
| Yield (purified) | ~0.3 g/L transient CHO |
| Concentration | 8.68 mg/ml (PBS pH |
| (A280) | 7.2) |
| Endotoxin (LAL) | <0.1 EU/mg |

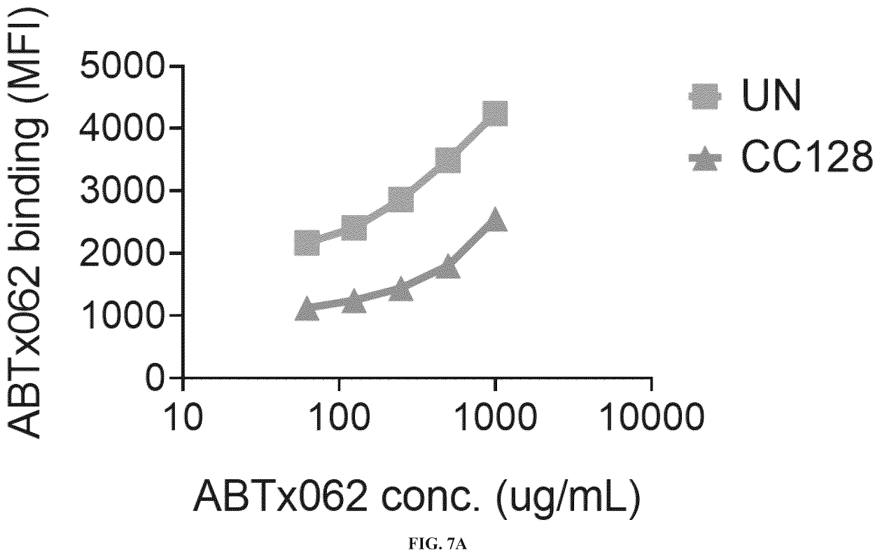

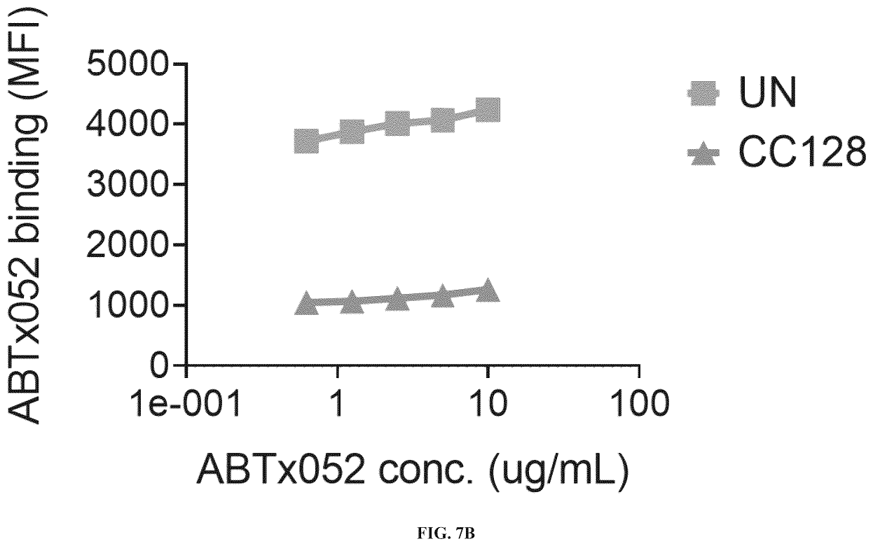

Experiments were undertaken to evaluate the binding of ABTx062 and ABTx052 to CD34+ cells edited using a base editor system containing ABE8.8 and CC128 sgRNA (FIGS. 7A and 7B). ABTx062 and ABTx052 bound cells expressing wild type CD117. The edited cells contained a 5G+7G edit (referenced to the following protospacer sequence corresponding to the CC128 sgRNA: 5′-AAATA₅TA₇ATAGCTGGCATCA-3′ (SEQ ID NO: 830)) to a CD117 polynucleotide sequence, which corresponded to the amino acid alterations Y259C and N260D. As shown in FIGS. 7A and 7B, mAbs ABTx062 and ABTx062 bound to unedited cells and showed a loss of binding to the CD34+ hematopoietic stem and progenitor cells (HSPCs) edited using gRNA CC128.

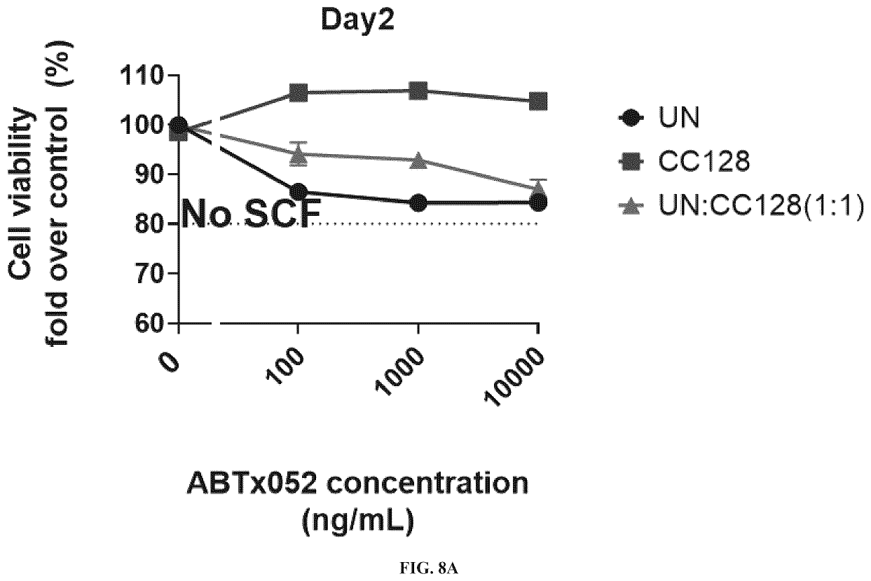

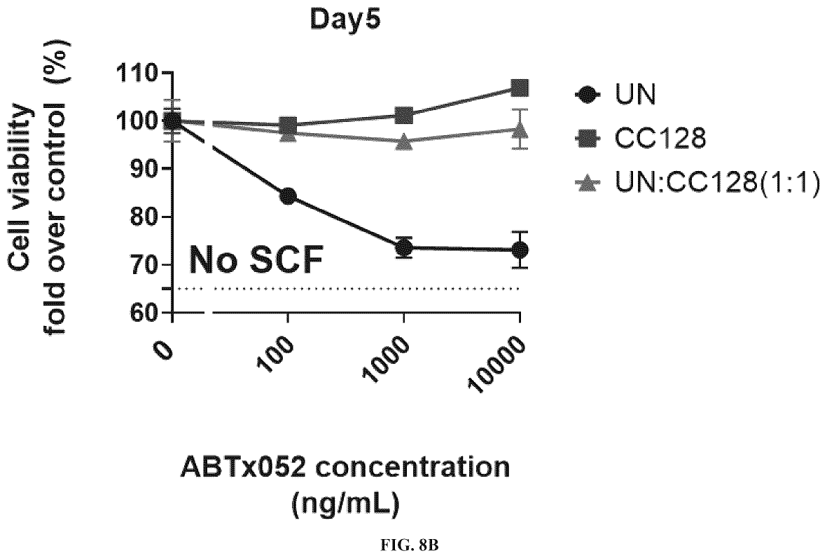

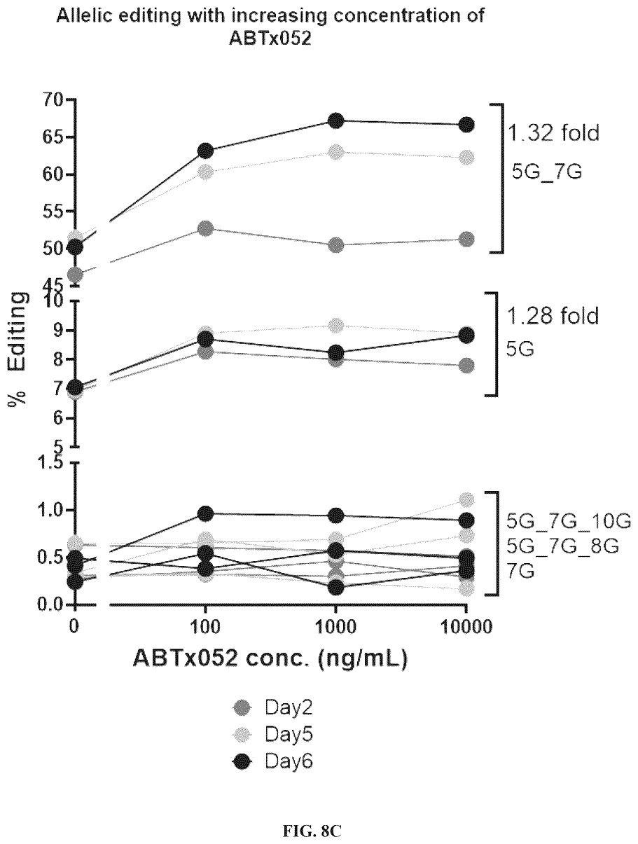

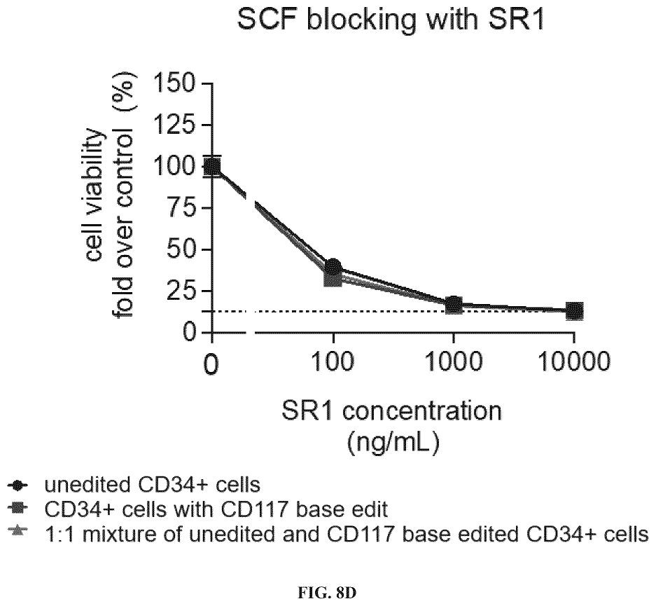

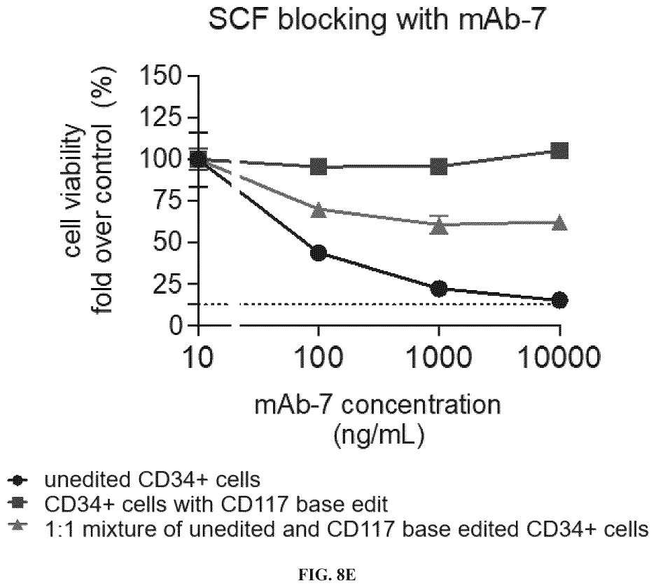

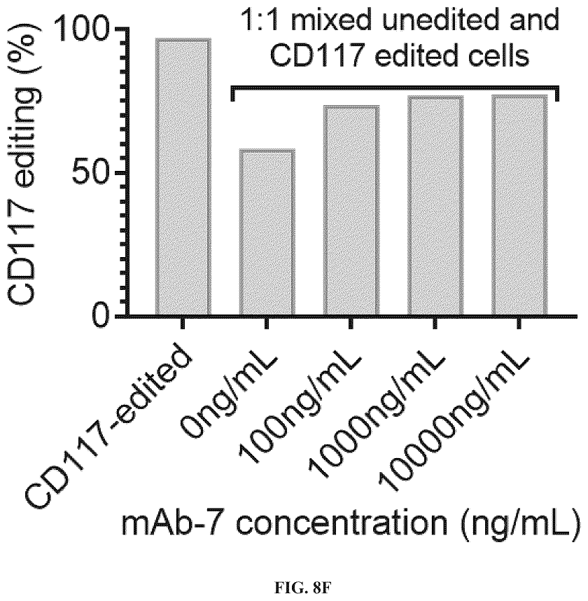

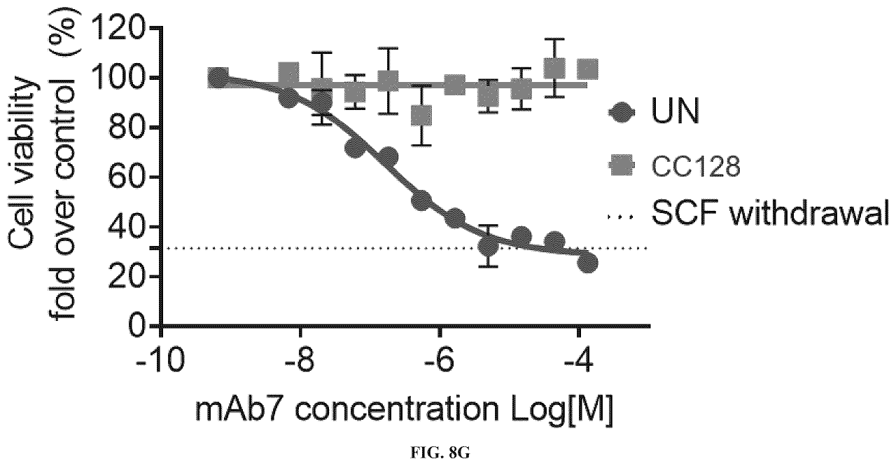

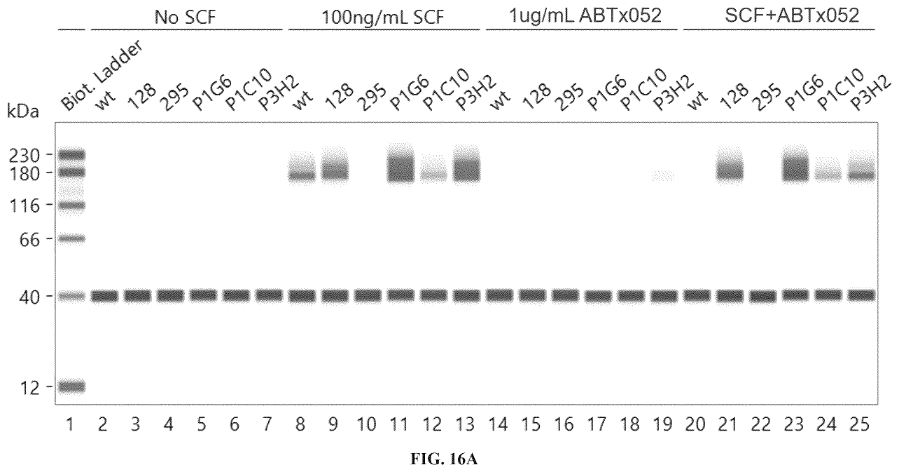

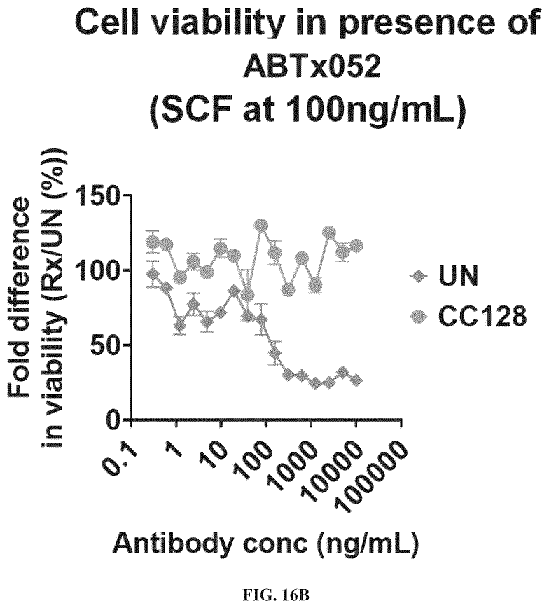

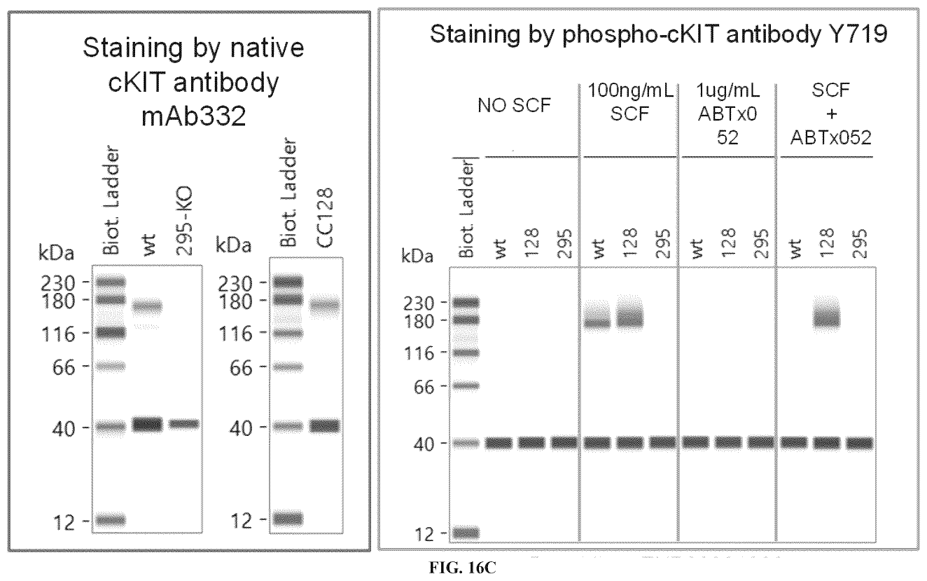

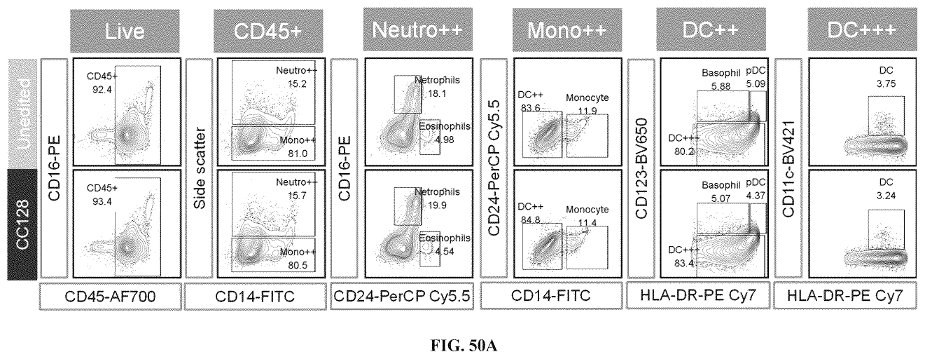

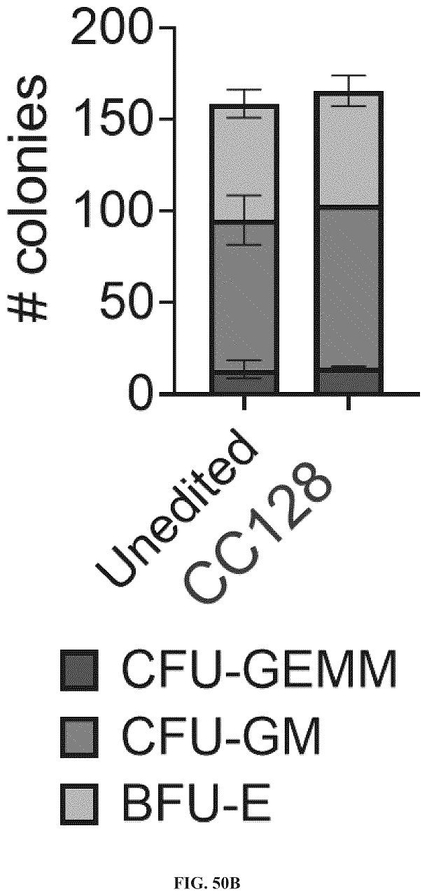

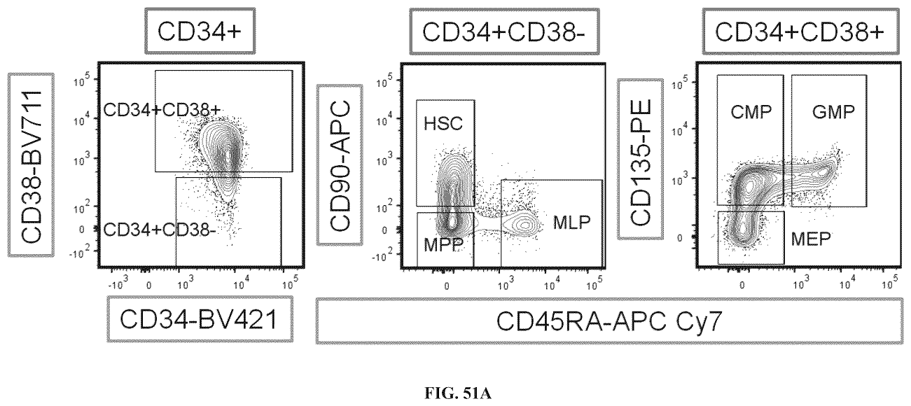

Next, experiments were undertaken to demonstrate that CD117 polypeptides containing an epitope engineered using the CC128 guide were protected against ligand blocking by ABTx052 in vitro and cells expressing the altered CD117 polypeptides had a proliferative advantage in the presence of the aBTx052 antibody relative to cells expressing a wild type CD117 polypeptide (FIGS. 8A-8G). Cell viability of primary CD34+ derived cells edited using ABE8.8 in combination with the CC128 guide and cultured in the presence of SCF at increasing concentrations of ABTx052 Ab was preserved. Long-term HSC, multi-potent progenitor, common myeloid progenitor, granulo-monocytic progenitor, and megakaryocytic-Erythrocytic progenitor subpopulations of hematopoietic stem cells were all sensitive to antibody treatment using ABTx135 (ABTx052 containing an LALADA alteration in the Fc domain) (FIGS. 51A and 51B). Viability in the absence of SCF was also preserved (FIGS. 8A and 8B). Cells expressing CD117 modified using the CC128 guide showed improved cell viability in the presence of ABTx052 relative to unedited cells (FIG. 16B). Unedited cells exposed to ABTx052 exhibit viability that resembles complete withdrawal of SCF (dotted line in FIGS. 8A and 8B). Next-generation DNA sequencing (NGS) of 1:1 mixes of unedited and edited cells demonstrated an increase in editing frequency, which demonstrated enrichment of cells harboring the target CD117 edit (5G+7G; Y259C+N260D) (FIGS. 8D and 8F). FIGS. 8D and 8E show that contacting cells expressing a wild type CD117 polypeptide (“unedited cells”) with ABTx052 mimicked complete SCF withdrawal and led to ˜85% loss of viability in vitro, while edited cells showed increased viability relative to the unedited cells. Therefore, the edited cells maintained viability upon exposure to ABTx052.

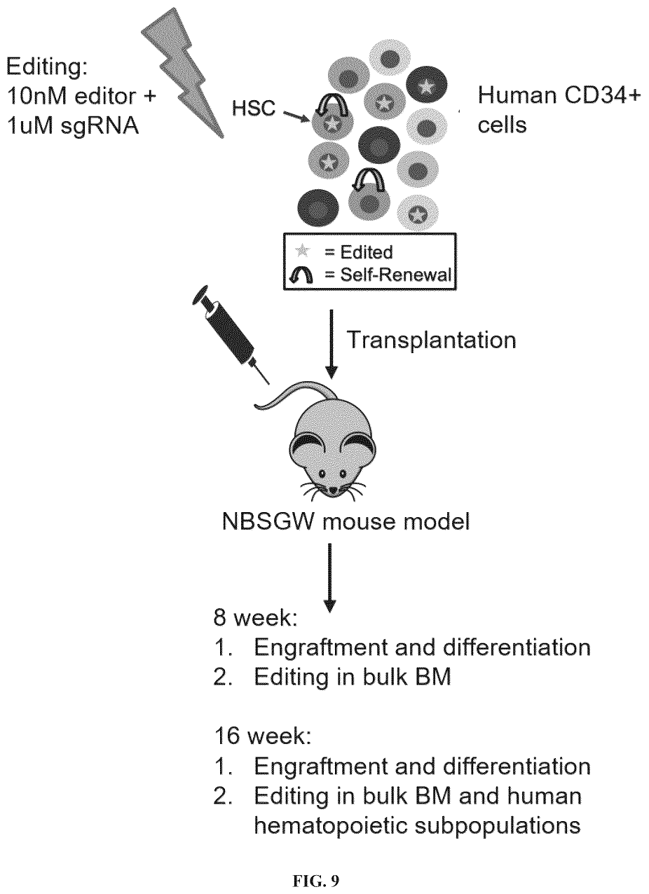

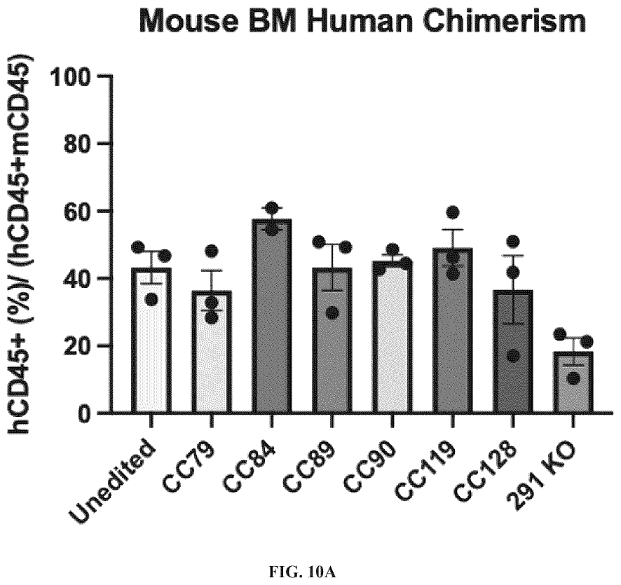

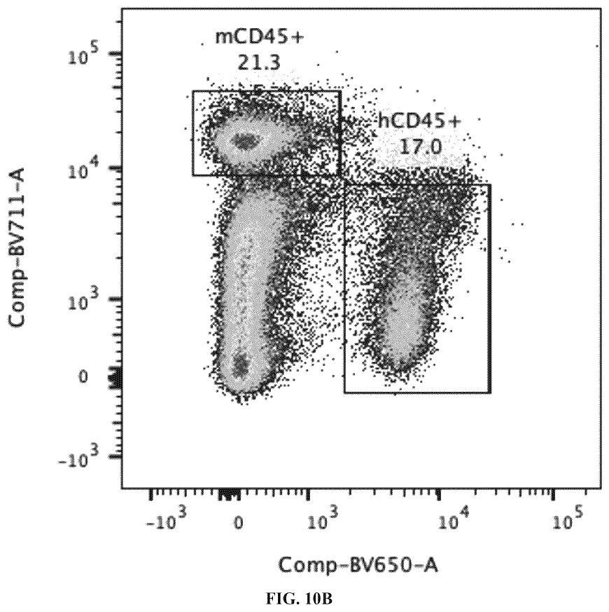

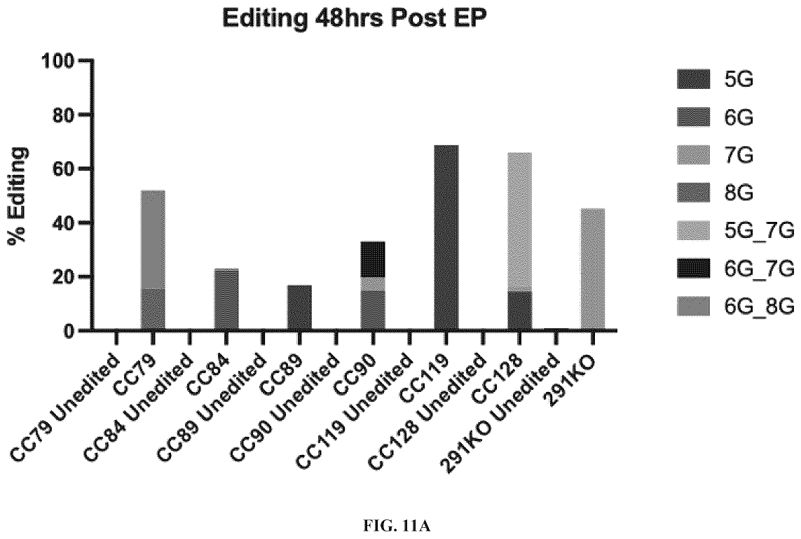

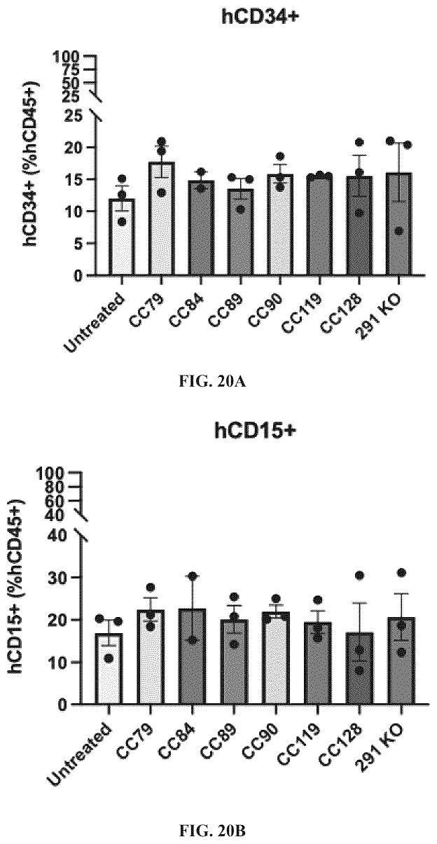

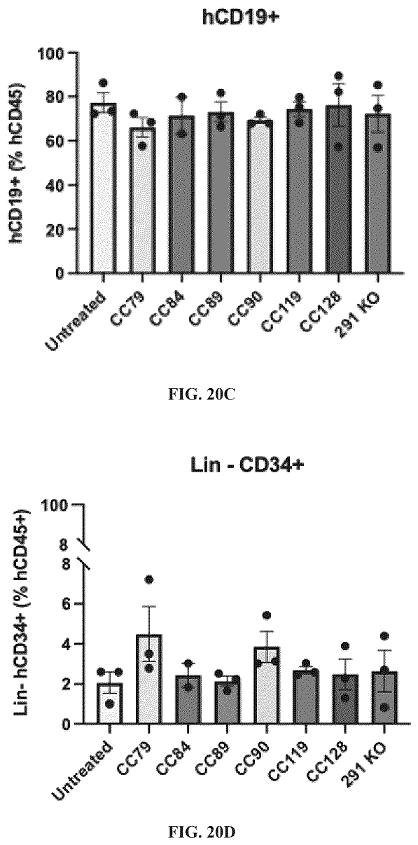

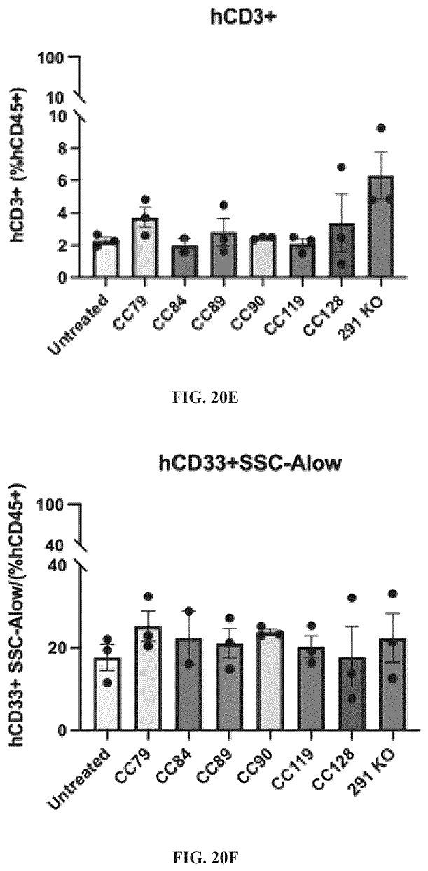

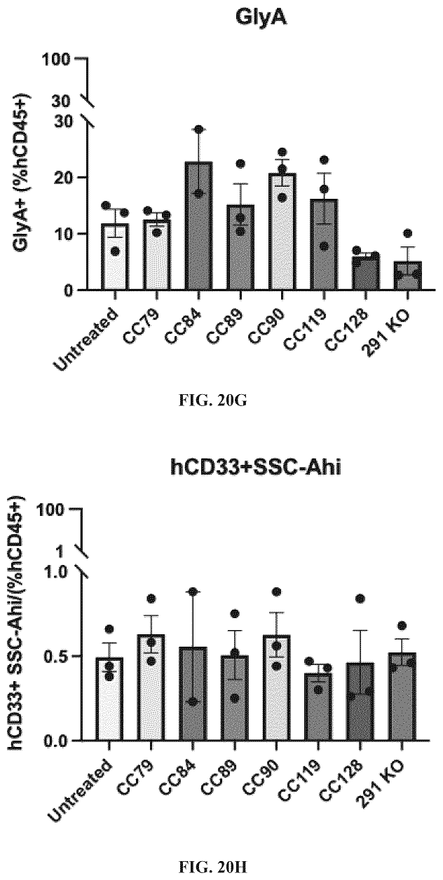

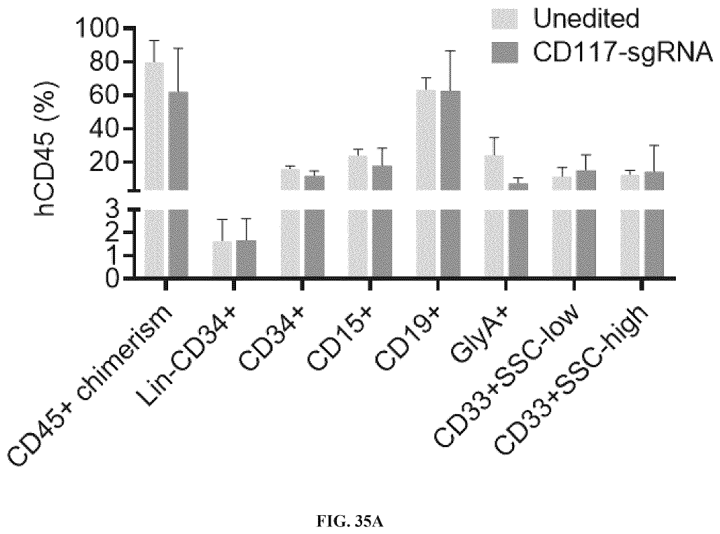

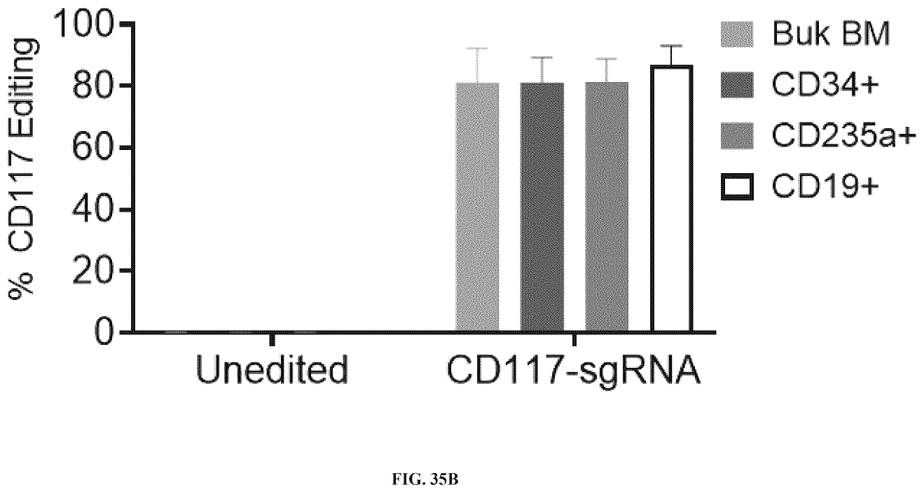

An in vivo study was undertaken in mice to evaluate engraftment and function of cells expressing a CD117 polypeptide edited using the CC128 guide in combination with ABE8.8. The experimental design is described in Table 10 and in FIG. 9. The CD117 variants evaluated are described in Table 12. Engineered hematopoietic stem cells were transfected with the guide and mRNA encoding ABE8.8 using electroporation. Engraftment of the CD117 variant cells in NBSGW mice was not altered by the base editing, as shown in FIGS. 10A, 10B, 20A-20H, 35A, and 35B, where engraftment of the edited cells was found to be approximately the same as or greater than that of unedited cells across cellular compartments. Comparable multi-lineage reconstitution was observed for unedited and CD117-edited CD34+ cells (FIG. 35A). High editing retention was observed for the CD117-edited cells within different bone marrow hematopoietic compartments indicating long-term persistence of CD117 editing (FIG. 35B). As demonstrated in Table 11, differentiation potential of the cells was not altered by the base editing carried out to alter the CD117 polypeptide sequence. Methods for differentiation of CD34+ cells are described in Giarratana M C et al. Proof of principle for transfusion of in vitro-generated red blood cells. Blood. 2011 Nov. 10; 118 (19): 5071-9. doi: 10.1182/blood-2011-06-362038. Epub 2011 Sep. 1. PubMed PMID: 21885599; PubMed Central PMCID: PMC3217398. Also, as shown in FIGS. 11A and 11B, editing of the CD117 polynucleotide was retained in bulk bone marrow (BM) at the 8-week timepoint.

TABLE 10
Experimental design.

| | Treatment | | | |
|---|---|---|---|---|
| | (guides | | | |
| | used to | | | |
| | edit the | | | N for each |
| Group | cells) | Dose (cells/mouse) | Route | timepoint |
| 1 | Unedited | 3.00E+05 | IV | 8-wk: 3 |
| | | | | 16-wk: 5 |
| 2 | CC79 | 3.00E+05 | IV | 8-wk: 3 |
| | | | | 16-wk: 5 |
| 3 | CC84 | 3.00E+05 | IV | 8-wk: 3 |
| | | | | 16-wk: 5 |
| 4 | CC89 | 3.00E+05 | IV | 8-wk: 3 |
| | | | | 16-wk: 5 |
| 5 | CC90 | 3.00E+05 | IV | 8-wk: 3 |
| | | | | 16-wk: 5 |
| 6 | CC119 | 3.00E+05 | IV | 8-wk: 3 |
| | | | | 16-wk: 5 |
| 7 | CC128 | 3.00E+05 | IV | 8-wk: 3 |
| | | | | 16-wk: 5 |
| 8 | CC291 KO | 3.00E+05 | IV | 8-wk: 3 |
| | | | | 16-wk: 5 |

“IV” is an abbreviation for intravenous.

TABLE 11
BT-499 8-week engraftment.

Average frequencies of cell populations in
BM (% hCD45+)

| Group | Lin- | hCD235a+ | hCD15+ | hCD19+ |
|---|---|---|---|---|
| (variant) | hCD34+ | (erythroid) | (myeloid) | (B cell) |
| Untreated | 2.1 | 11.9 | 16.9 | 77.3 |
| CC79 | 4.5 | 12.5 | 22.4 | 66.1 |
| CC84 | 2.4 | 22.8 | 22.8 | 71.5 |
| CC89 | 2.1 | 15.2 | 20.1 | 73.1 |
| CC90 | 3.9 | 20.8 | 22 | 69.3 |
| CC119 | 2.7 | 16.3 | 19.5 | 74.4 |
| CC128 | 2.5 | 6 | 17.1 | 76.2 |
| 291KO | 2.6 | 5.2 | 20.7 | 72.3 |

TABLE 12
Variant edits, where the location of each edit is indicated
with referenced to the distance of the nucleotide from the
5′ end of the corresponding spacer sequence (see Table 1).

| Variant | Intended | | | | |
|---|---|---|---|---|---|
| ID | variant | 5G | 6G | 7G | 8G |
| CC79 | M351T | | M351T | | Y350Y |
| CC84 | Q79R | | Q79R | | N80D |
| CC89 | T144A | T144A | | | N145D |
| CC90 | E81G | | E81G | E81G | |
| CC119 | D45G | D45G | | | |
| CC128 | Y259C/N260D | Y259C | | N260D | |

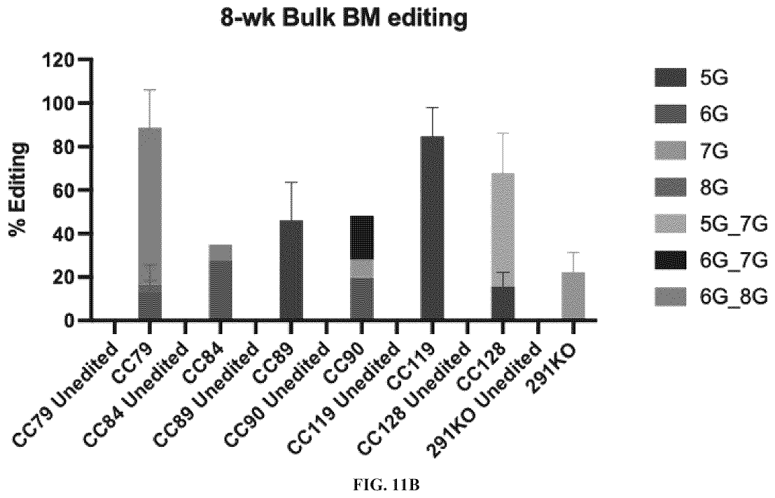

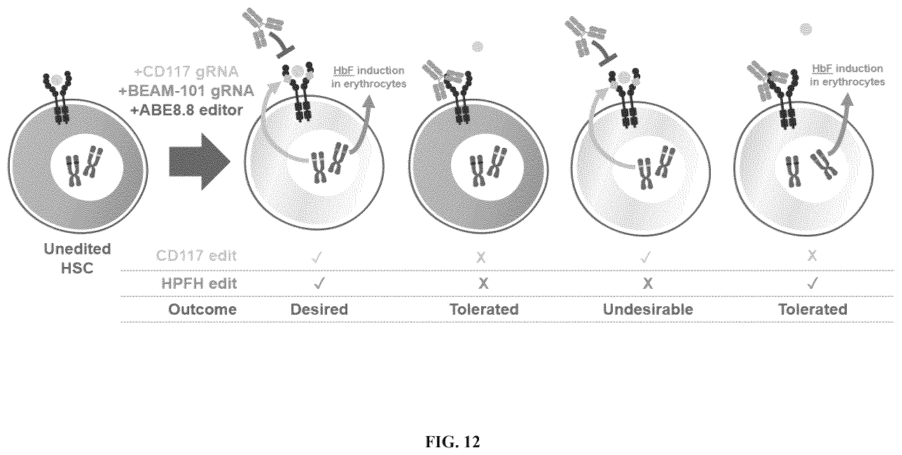

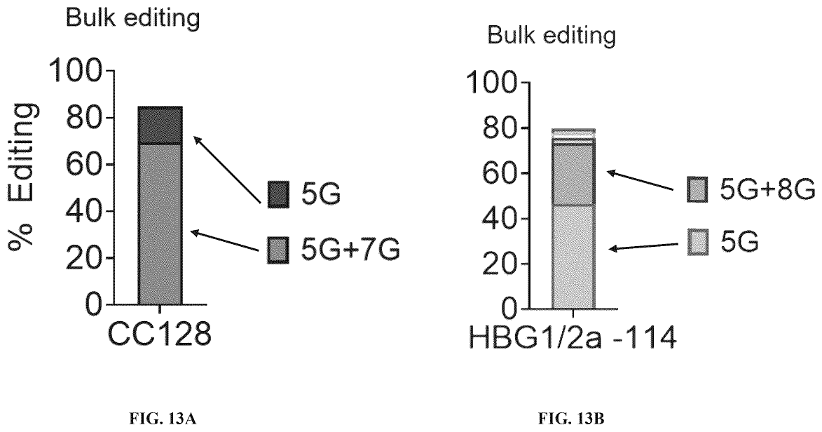

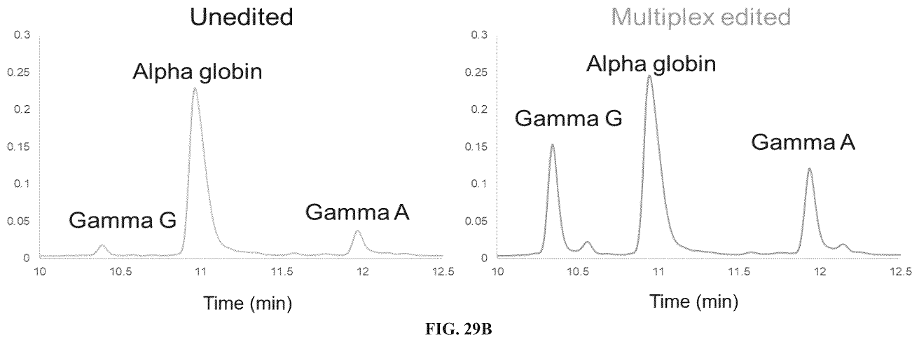

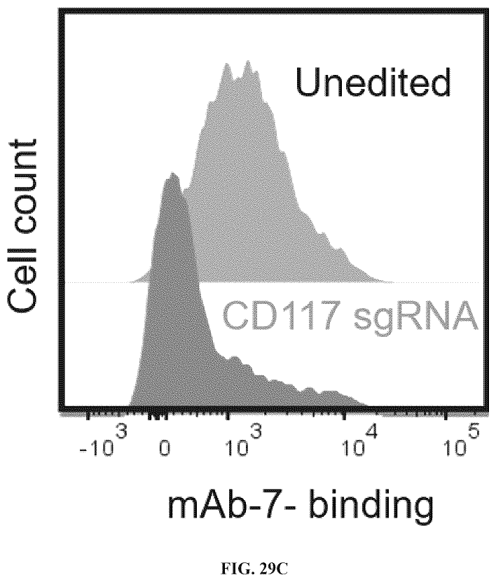

Next, primary CD34+ hematopoietic stem and progenitor cells (HSPCs) were base edited in multiplex using ABE8.8 in combination with sgRNA CC128 and sgRNA_015 (HBG1/2-114 edit) (FIGS. 12, 13A, and 13B). Multiplex editing with sgRNA HBG1/2a-114 and gRNA CC128 yielded highly efficient base editing (>80% and ˜70%, respectively) (FIGS. 13A and 13B). An additional ˜15% editing of 5G alone (CD117 Y259C) was also detected. Over 60% gamma induction was detected for in vitro erythroid differentiated (IVED) cells that were multiplex edited (similar to those achieved single-plex sgRNA_015 editing alone). Further, as shown in FIG. 29A, multiplex (>85% or >75%) editing efficiencies were equivalent to those for non-multiplex edits. The outcomes for editing of the CD117 polynucleotide were favorable: >87% bi-allelic, 1.5% mono-allelic, and 1.5% unedited. Multiplex editing did not hinder gamma globulin induction and was associated with >55% gamma-globin induction (FIG. 29B). The multiplex edited cells escaped recognition by mAb-7 (FIG. 29C). Over 60% of gamma globin induction was detected in in vitro differentiated erythroid cells edited in multiplex, which was similar to levels with a single-plex HBG1/2 edit. In some cases, about 80% bi-allelic CD117 editing was observed and near complete editing of the HBG1/2 locus in HSCs. It was confirmed that multiplex editing was achieved in the cells by confirming that cells containing the HBG1/2-114 edit also contained an altered CD117 polynucleotide.

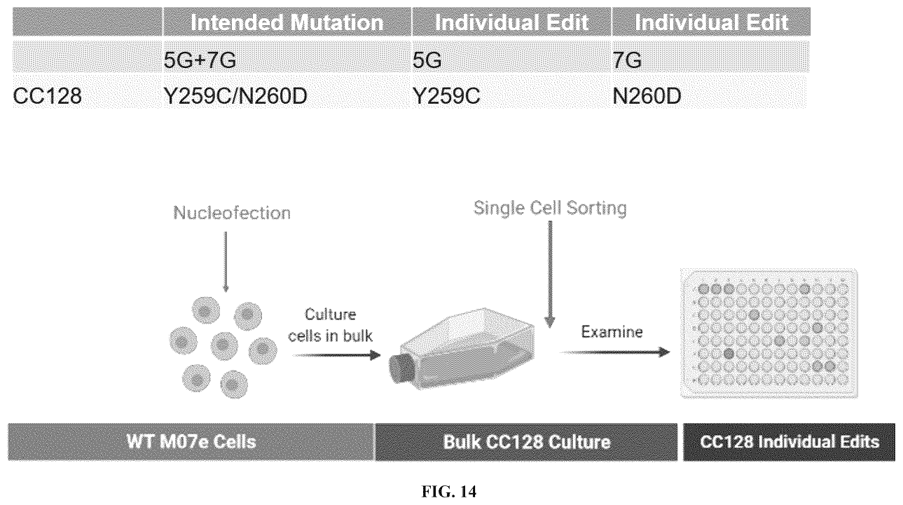

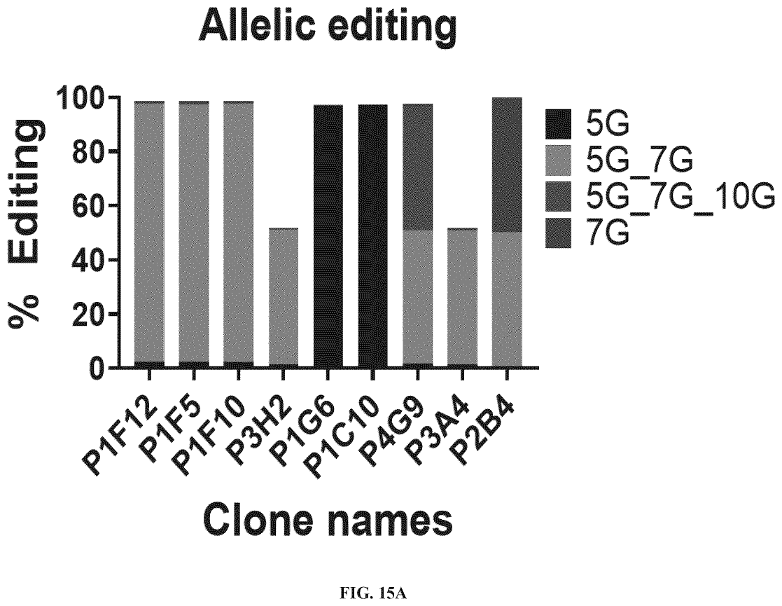

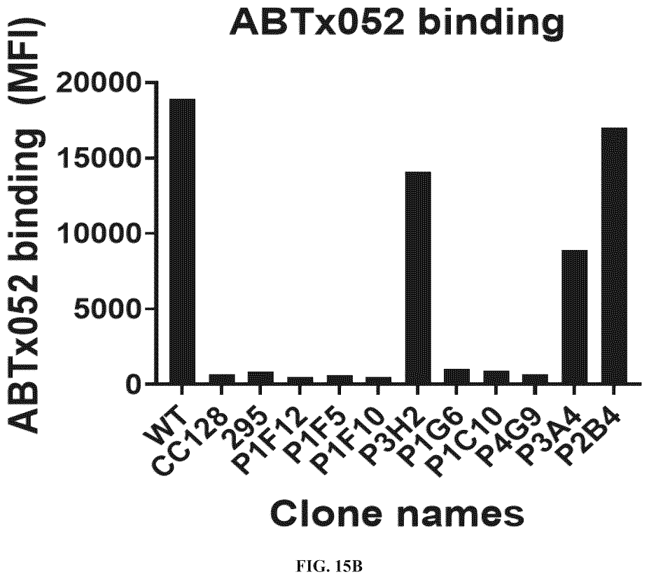

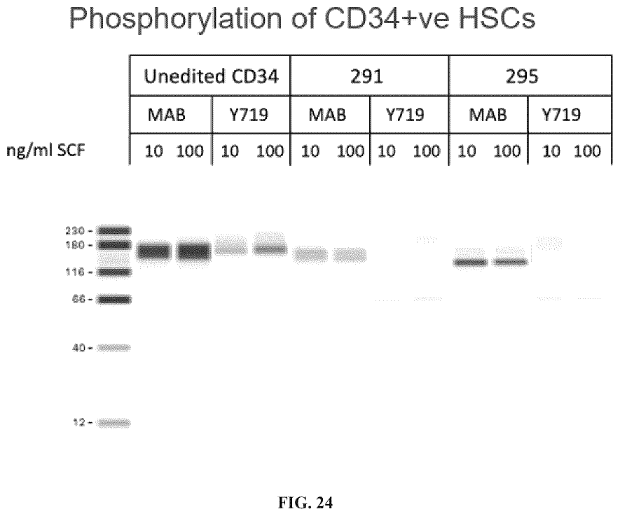

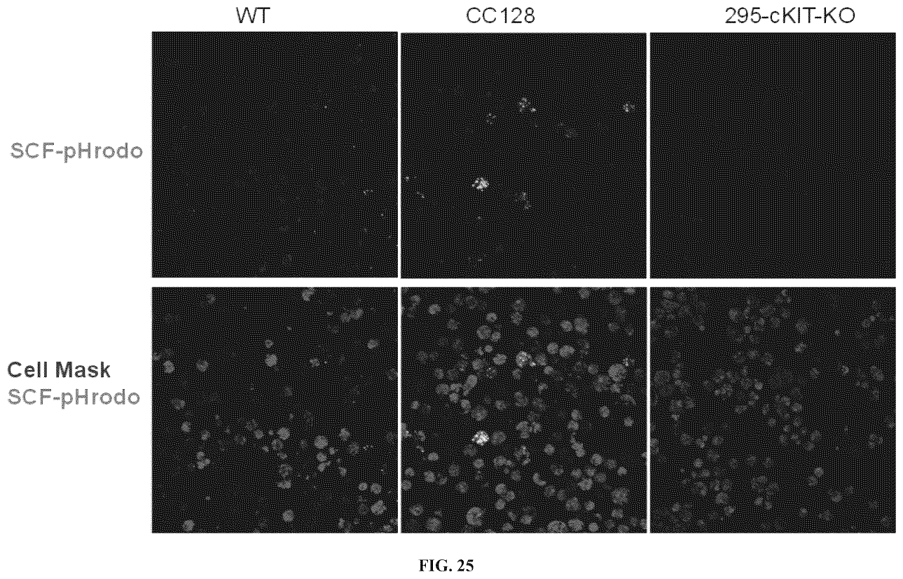

Experiments were next undertaken to demonstrate that c-KIT/CD117 polypeptides edited using the CC128 guide were functional in vitro. Given the about 15% occurrence of the 5G edit alone (CD117 Y259C) in cells edited using the CC128 guide, these CD117 variants were individually investigated for normal functionality in addition to the 5G+7G combined edit (CD117 Y259C+N260D). The experimental design for generating single cell clones for de-risking individual edits introduced using the CC128 guide used in combination with ABE8.8 is illustrated in FIG. 14. Wild type MO7e cells were nucleofected with mRNA encoding ABE8.8 and the CC128 guide polynucleotide. Transfected cells were then cultured in bulk and sorted to isolate cells containing individual edits to the CD117 polynucleotide. Clones that were isolated and evaluated are described in Table 13. Allelic editing measured for each clone is shown in FIG. 15A. ABTx052 binding to each clone was measured and many of the clones showed reduced binding relative to wild type cells expressing an unedited CD117 polypeptide (FIG. 15B). ABTx052 (alternatively referred to as “mAb-7”), bound CD117 with high affinity, did not bind to the edited CD117 variant prepared using the guide CC128 as purified protein or expressed in M07e cells, and blocked SCF binding to wild type CD117. The KD(M) of ABTx052 (mAB-7) for human CD117 was <1.0E-12 and the KD(M) of ABTx052 for cyno CD117 was <1.0E-12. Phosphorylation of CD117 expressed by the clones P3H2, P1G6, P1C10, and CC128 was measured in the presence of SCF and/or ABTx052 (FIGS. 16A-16D). Wild type CD117 was also phosphorylated, and no phosphorylation of CD117 was measured in cells with knock-out edits to the CD117 polynucleotide sequence (FIG. 24). Therefore, the c-KIT/CD117 polypeptides using the CC128 guide were functional.

TABLE 13
M07e single clones.

| | |
|---|---|
| Clone | |
| Name | Allelic Editing Composition |

| | | |
|---|---|---|
| P1F12 | 5G + 7G | Biallelic |
| P1F5 | 5G + 7G | Biallelic |
| P1F10 | 5G + 7G | Biallelic |
| P3H2 | 5G + 7G | Monoallelic |
| P1G6 | 5G | Biallelic |
| P1C10 | 5G | Biallelic |
| P4G9 | 5G + 7G + 10G | |
| P3A4 | 5G + 7G | Monoallelic |
| P2B4 | 5G + 7G/7G | Monoallelic |
| WT | N/A | N/A |
| CC128 | 5G + 7G | Biallelic |
| CC295 | cKIT Knockout | Biallelic |

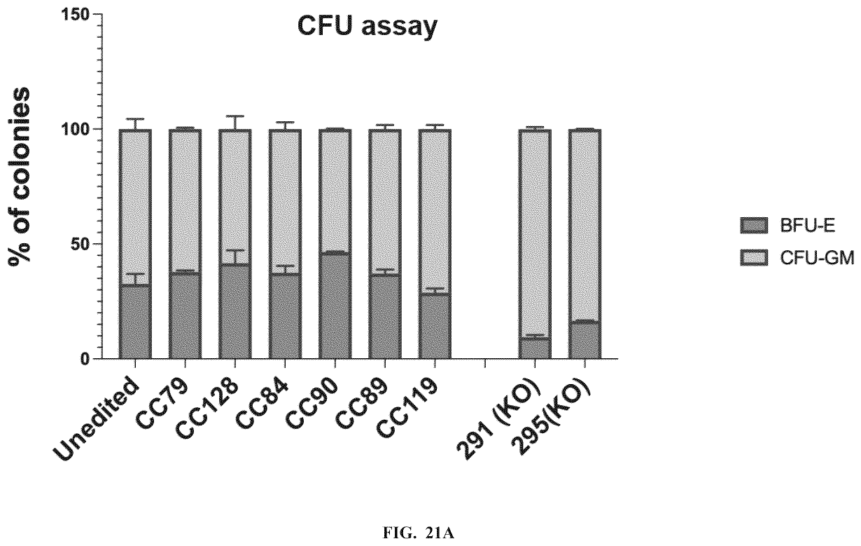

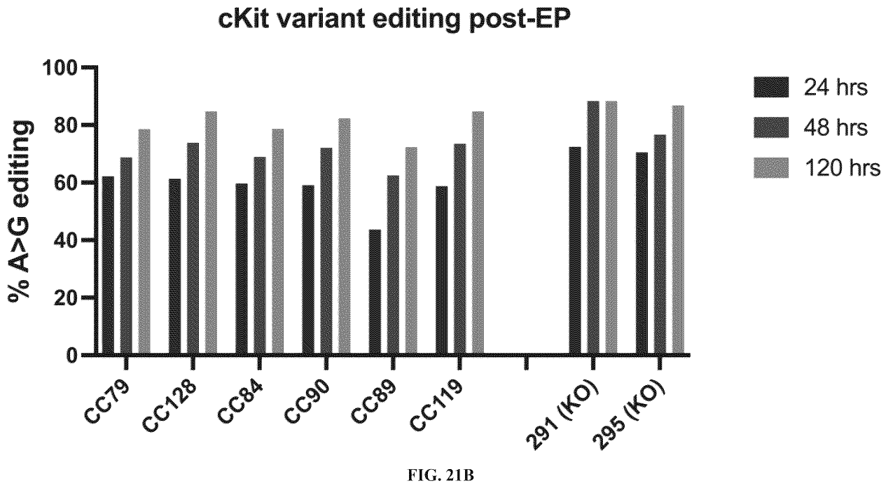

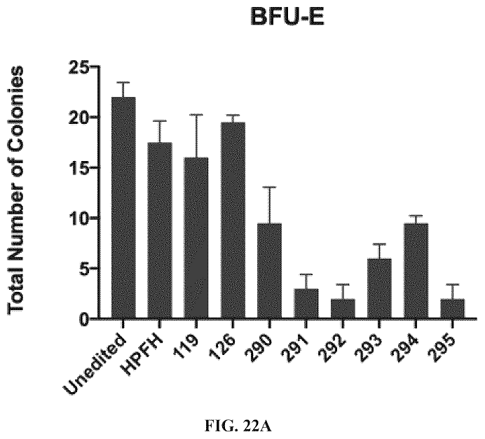

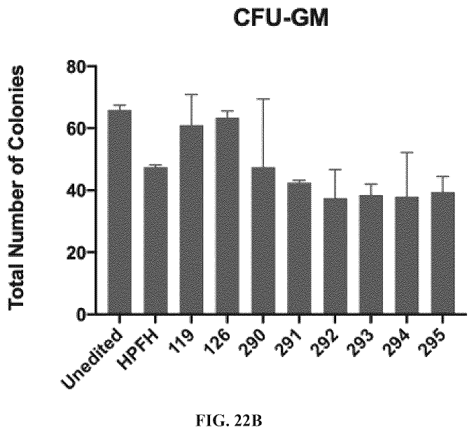

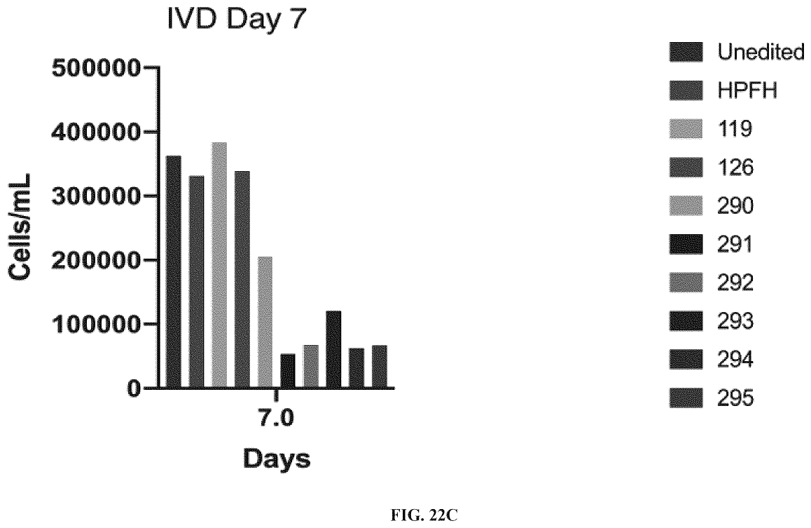

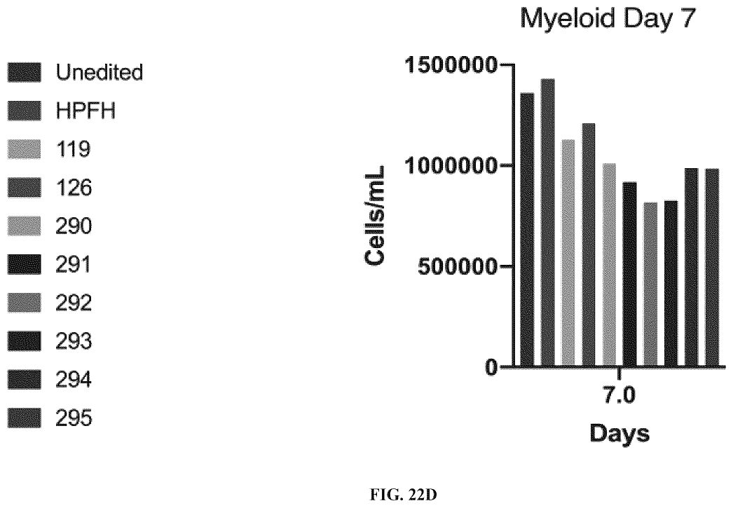

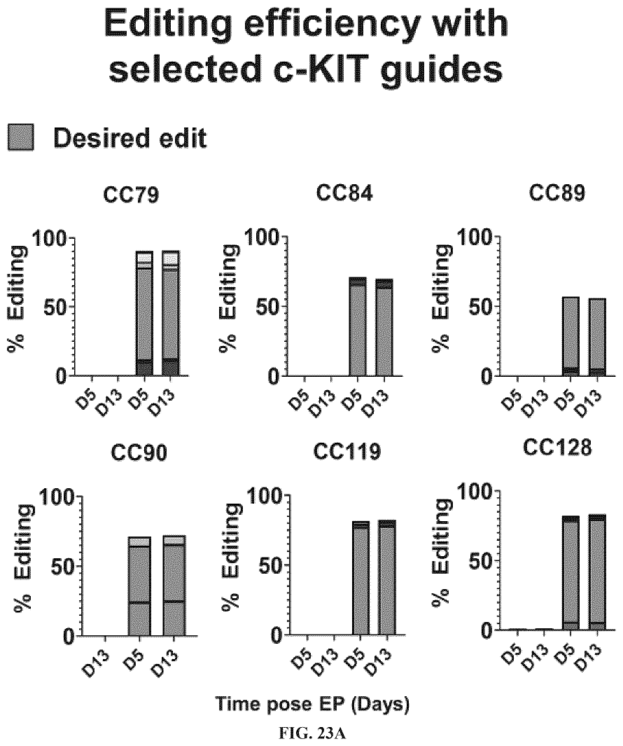

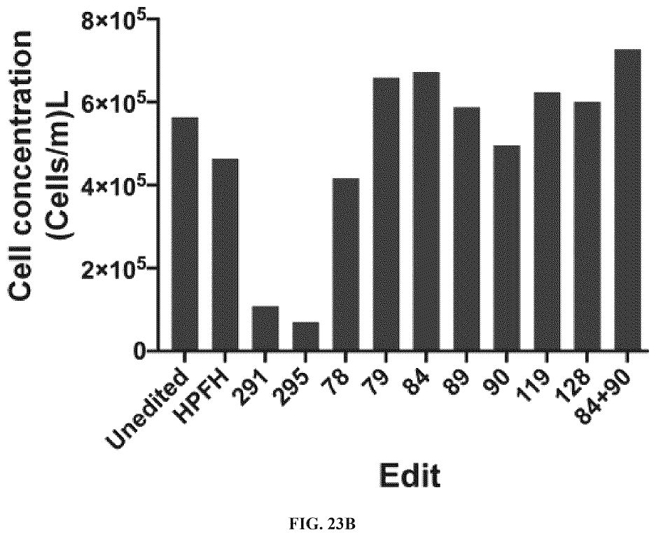

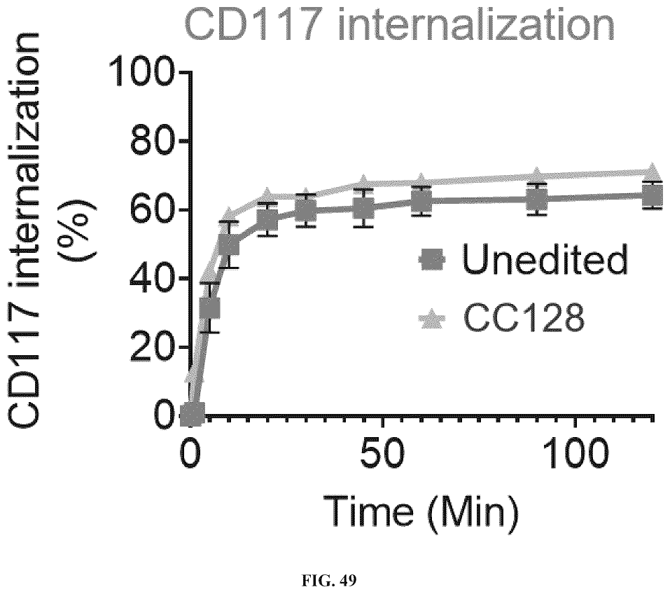

Further experiments were undertaken to determine that altered CD117/c-KIT receptors reflected the life cycle of the CD117 receptor. Editing efficiencies corresponding to guides for altering a CD117 polynucleotide are shown in FIG. 23A. Target edits carried out using the guides are described in Table 14. The lifecycle of the CD117 receptor includes ligand binding, dimerization, trans-phosphorylation at the juxta membrane region, phosphorylation in the intracellular kinase domain, ubiquitination, internalization, and proteolytic degradation. The following assays were completed to evaluate receptor biology: phosphorylation and internalization assays. The following hematopoietic stem cell (HSC) assays were also completed: colony forming units (CFU) (FIG. 21A), in vitro differentiation (IVD) (FIG. 22C), myeloid (FIG. 22D), and in vivo multilineage reconstitution. Mast cell activation was also evaluated. As discussed above, the altered CD117 polypeptides were phosphorylated in the presence of SCF. It was also determined that CD117 expressed by cells edited using the guide CC128 was internalized by the cells upon binding to SCF (FIGS. 25 and 49). Highly efficient, bi-allelic editing was achieved in CD34+ cells to yield variants having normal colony forming unit capacity (FIGS. 21A and 21B). Disrupting CD117 function through knock-out impacted the colony forming ability of the erythroid population. Over 85% bi-allelic editing was achieved using the guides CC79, CC128, CC84, CC90, CC89, and CC119 (FIG. 21B). CD117 knockout profoundly affected erythroid in vitro differentiation (FIGS. 22A-22D and 23B), but edits carried out using the guides 78, 79, 84, 89, 90, 119, and 128 did not (FIG. 23B). CFU assays indicated that editing CD117 had minimal effect on myeloid colony formation (FIG. 21A). Therefore, the CD117 mutations carried out using the guides CC78, CC79, CC84, CC89, CC90, CC119, CC128, and CC84+CC90 did not disrupt HSPC function and differentiation (e.g., into myeloid or erythroid lineages) (see, e.g., FIGS. 50A and 50B).

TABLE 14
Mutations corresponding to guides.

| Guide | Mutation/edits |
|---|---|
| CC78 | Y418C |
| CC79 | M351T |
| CC84 | Q79R |
| CC90 | E81G |
| CC89 | T144A |
| CC119 | D45G |
| CC128 | Y259C + N260D |
| 291 | A to G at splice site |
| | (KO) |
| 295 | A to G at splice site |
| | (KO) |

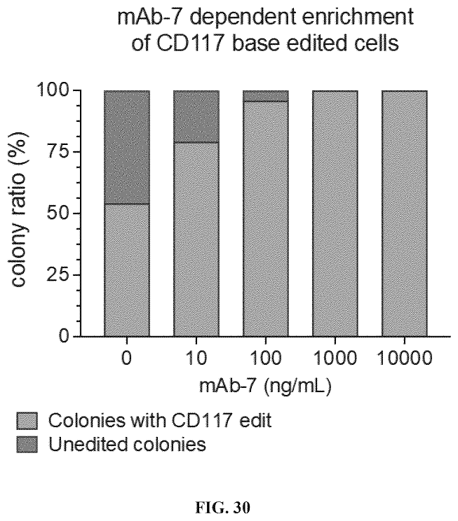

Experiments were undertaken to demonstrate that CD34+ hematopoietic stem cells edited using ABE8.8 in combination with the guide CC128 were enriched in a mixed culture containing the base edited and unedited cells when grown in the presence of ABTx052 (alternatively referred to as mAb-7) (FIG. 30). A 1:1 mixture of unedited and edited cells was plated in semi-solid media in the presence of varying concentrations of ABTx052. At day 14, individual erythroid colonies (BFU-E) were picked and sequenced (24 total for each concentration of ABTx052). Stem cell factor (SCF) blocking by ABTx052 significantly inhibited erythroid colony formation by unedited cells. In the absence of ABTx052, an even distribution of edited and unedited colonies was observed. Increasing concentration of ABTx052 yielded a correspondingly higher proportion of edited colonies. 100% of colonies at ABTx052 concentration 100 ng/ml or higher contained a 5G or 5G_7G edit. Very few BFU-E colonies were observed when the cells were incubated with the antibody SR1 as a positive control, which blocks SCF binding to CD117 in both edited and unedited cells. Treatment of unedited HSPCs with mAB-7 in vitro resulted in a >85% reduction in viability of the cells, while CD117-edited cells remained unaffected.

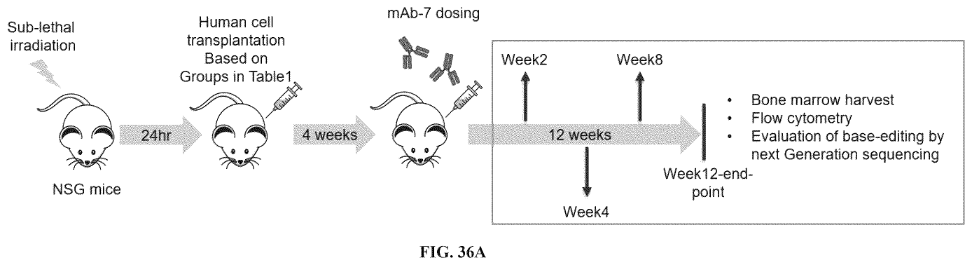

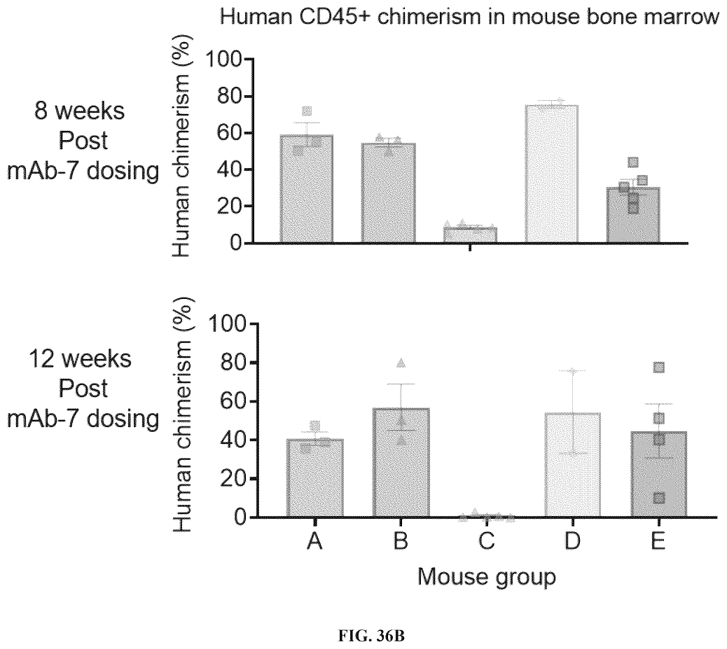

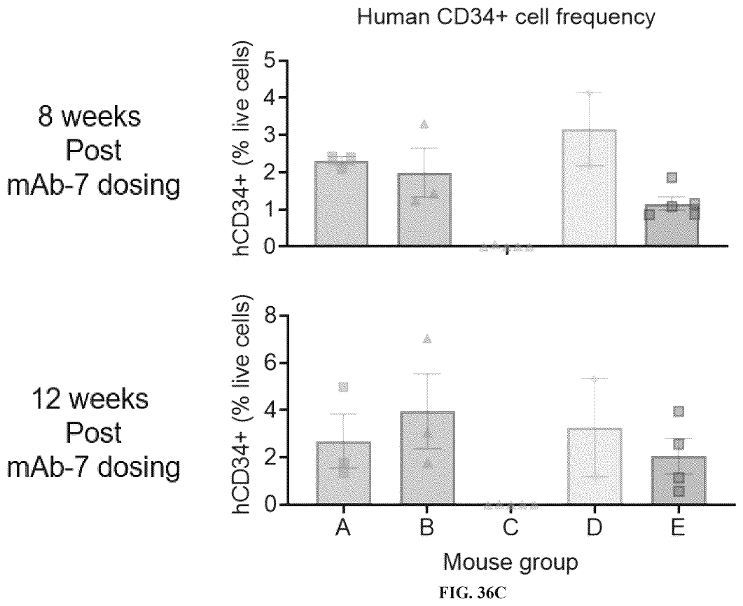

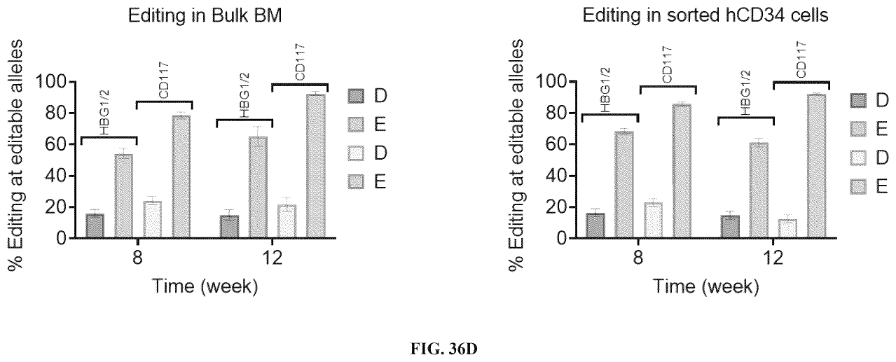

Experiments were undertaken to demonstrate through xenotransplantation studies that CD117-edited HSPC were capable of multi-lineage hematopoietic engraftment in immunocompromised mice as shown in FIG. 36A. To evaluate anti-CD117-mAb-mediated myeloablation, NSG mice were first humanized with either unedited cells or a 1:1 mixture of unedited and multiplex edited (i.e., cells containing an altered CD117 gene and an altered HBG1/2 gene; 1e6 cells/mouse) human CD34+ cells. 4 weeks post-transplantation, mice were either left untreated, or treated with either isotype control antibody or mAb-7 according to the following mouse groups: A) unedited cells and no antibody treatment; B) unedited cells and treatment with a control isotype antibody; C) unedited cells and treatment with mAb-7; D) 1:1 ratio of edited and unedited cells and treatment with the control isotype antibody; E) 1:1 ratio of edited and unedited cells and treatment with mAb-7. Mouse bone marrow was harvested at 8 and 12 weeks post mAb treatment administration and was analyzed using flow cytometry (to measure overall CD45+ human chimerism or CD34+ cell frequency) (FIGS. 36B and 36C). Bulk bone marrow and sorted CD34+ cells were analyzed using NGS to evaluate editing retention (FIG. 36D). It was found that mAb-7 selectively depleted unedited cells form the bone marrow (BM) of mice transplanted with hCD34+ cells. Significant ablation of human chimerism was observed in mAb-7 treated mice transplanted with unedited CD34+ cells. The mAb-7 treatment led to in vivo selection of multiplex edited cells, as indicated by high levels of CD117 and HBG1/2 editing in antibody-treated mouse bone marrow.

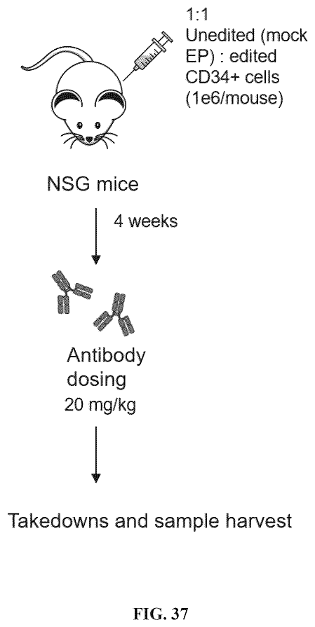

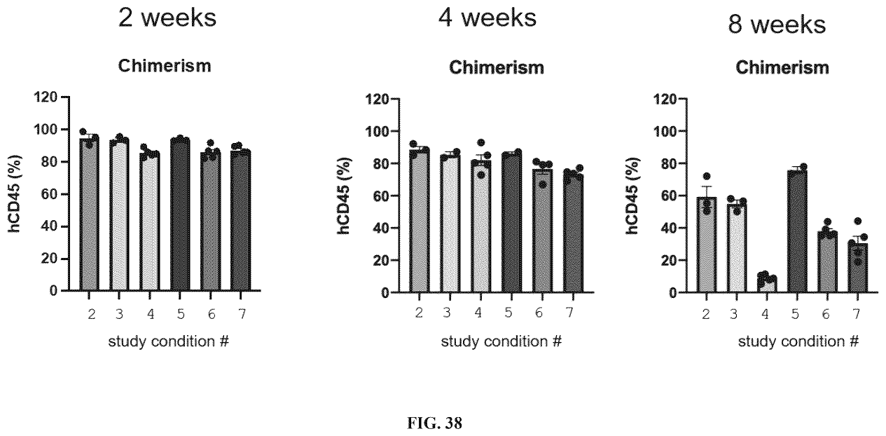

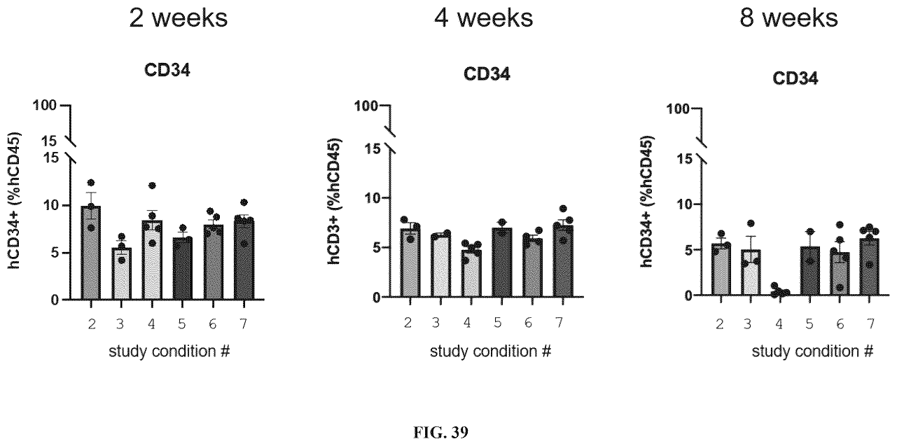

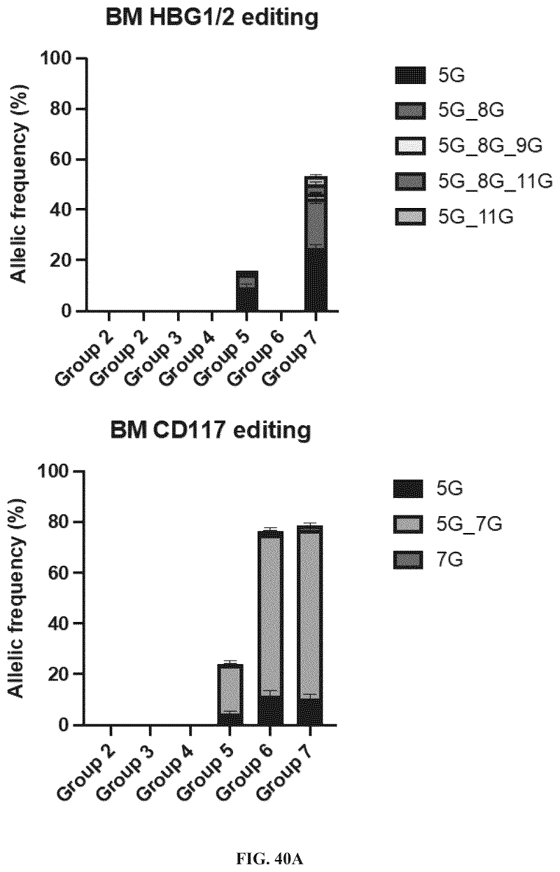

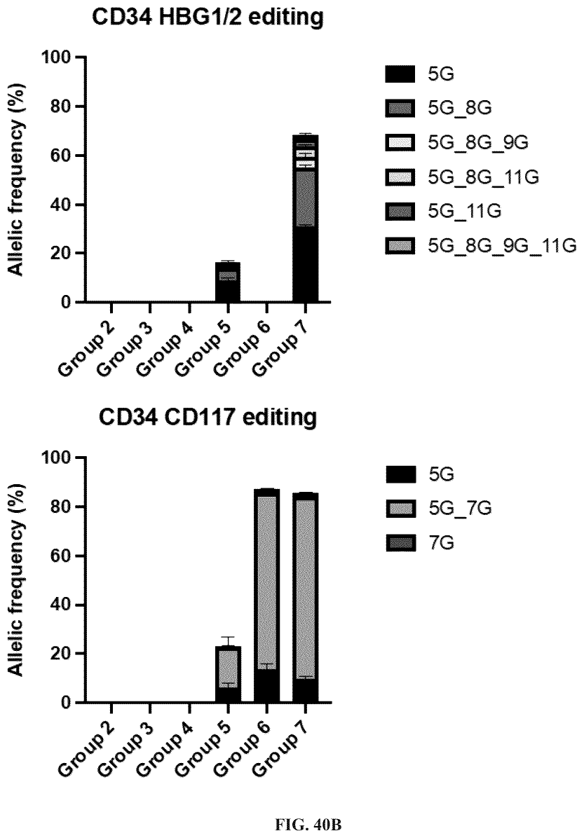

A further in vivo experiment was undertaken (see FIG. 37) to evaluate ABTx052-mediated enrichment of multiplex edited cells in NSG mice. Table 15 below describes what cells the mice were administered and the number of mice evaluated at each time point listed. The following was evaluated at each time point: chimerism, HSC phenotyping, base edits observed in bulk bone marrow (BM) and Lin−CD34+, and CD117 staining. At 12 weeks post-antibody dosing, a globin analysis was completed for each mouse. Engraftment of the cells in the mice was measured as CD45+ cell chimerism, and it was observed that unedited cells were depleted in the mice following administration of ABTx052 (FIG. 38). Selective depletion of CD34+ stem cells was observed in mice engrafted with unedited HSCs and exposed to ABTx052 post-engraftment (FIG. 39), as demonstrated by ABTx052 treatment impacting CD34+ cell frequency within bone marrow of transplanted mice. Eight weeks post ABTx052 administration, sequencing of bulk bone marrow and lin-CD34+ cells revealed enrichment of cells containing a base-edited CD117 polynucleotide (FIGS. 40A and 40B). Increased levels of HBG1/2 and CD117 editing was measured in bulk BM and Lin−CD34+ populations of mice treated with ABTx052. Multiplex edited HSCs were retained in vivo.

TABLE 15
Treatment groups and number of mice evaluated at each indicated end point (TD).

| Group/Study | Cells | Ab | TD | TD | TD | TD | TD |
|---|---|---|---|---|---|---|---|
| Condition | (1e6/mouse) | treatment | (0 wk) | (2 wk) | (4 wk) | (8 wk) | (12 wk) |
| 1 | 1:1 ratio of | None | N = 3 | | | | |
| | unedited to | | | | | | |
| | multiplex | | | | | | |
| | edited cells | | | | | | |
| 2 | Unedited cells | None | N = 3 | N = 3 | N = 3 | N = 3 | N = 3 |
| 3 | Unedited cells | Isotype | | N = 3 | N = 3 | N = 3 | N = 3 |
| 4 | Unedited cells | ABTx052 | | N = 5 | N = 5 | N = 5 | N = 5 |
| 5 | 1:1 ratio of | Isotype | | N = 3 | N = 3 | N = 3 | N = 3 |
| | unedited cells | | | | | | |
| | to multiplex | | | | | | |
| | edited cells | | | | | | |
| 6 | 1:1 ratio of | ABTx052 | | N = 5 | N = 5 | N = 5 | N = 5 |
| | unedited cells | | | | | | |
| | to cells edited | | | | | | |
| | to alter a | | | | | | |
| | CD117 gene | | | | | | |
| | but not an | | | | | | |
| | HBG1/2 gene | | | | | | |
| 7 | 1:1 ratio of | ABTx052 | | N = 5 | N = 5 | N = 5 | N = 5 |
| | unedited cells | | | | | | |
| | to multiplex | | | | | | |
| | edited cells | | | | | | |
| 8 | 1:1 ratio of | None | | | | | N = 5 |
| | unedited to | | | | | | |
| | multiplex | | | | | | |
| | edited cells | | | | | | |

The above experiments demonstrate that the mAb-7 bound wild-type CD117 with high affinity but minimally to base edited CD117 variant protein. The also demonstrate that edited CD117 behaved normally in vitro vs wild-type in proliferation, differentiation, viability, and phosphorylation assays. Further, they demonstrate that CD117 base-edited CD34+ cells lead to multilineage reconstitution in rodent model comparable with unedited CD34+ cells. Also, Fc engineered mAb-7 did not induce mast-cell degranulation in vitro, and multiplex editing of cells using a CD117 sgRNA with therapeutic sgRNAs (e.g., HBG1/2) with a single ABE8 editor achieved >85% CD117 base editing in CD34+ cells also containing therapeutic edit. Further, Multiplex base edited CD34+ cells evaded mAb-mediated effects and SCF-ligand blocking, allowing for escape in vitro and in vivo. Finally, the mAb-7 selectively depleted unedited cells from the BM of mice transplanted with a 1:1 mixture of unedited and edited hCD34+ cells.

### Example 2: Development of an Engineered Stem Cell Antibody-Paired Evasion (ESCAPE) Approach for Engraftment of Hematopoietic Stem Cells (HSCs) Engineered to Express the Makassar Variant of Beta Hemoglobin

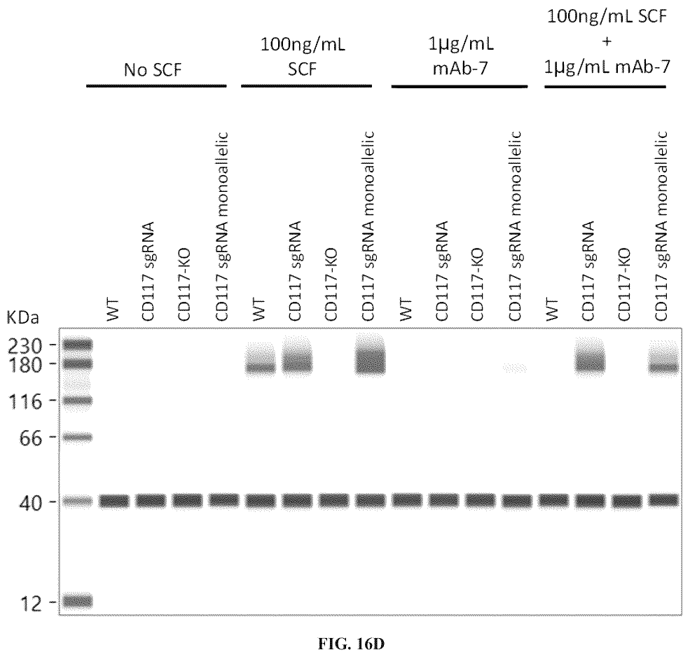

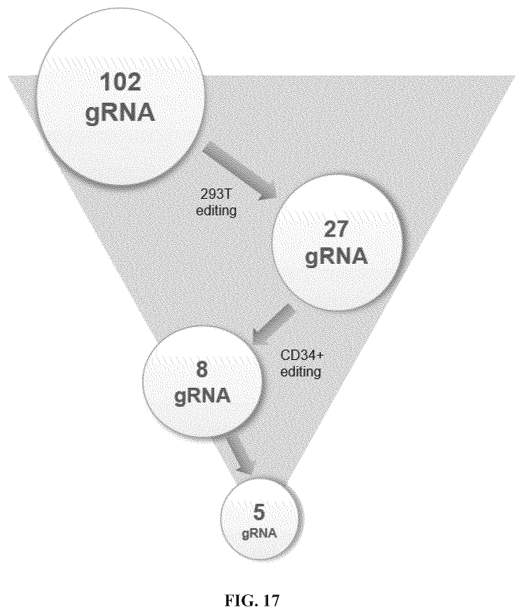

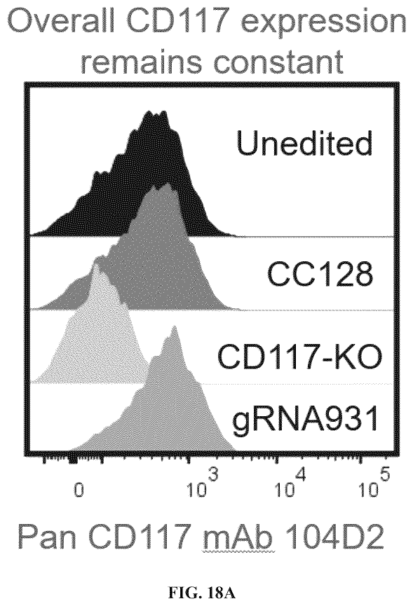

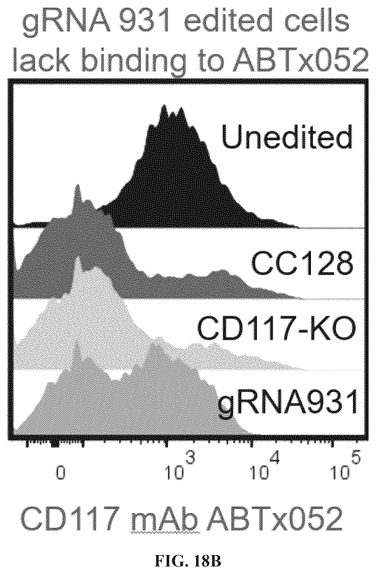

To develop an engineered stem cell antibody-paired evasion (ESCAPE) approach for engraftment of HSCs engineered to express a Makassar variant of beta hemoglobin, a set of 102 candidate guides were screened (FIG. 17). The guides were screen using the base editor ABE8.20-NRCH (see Table 16 below). The criteria for selection from guide screening in HEK293T cells were the following: 1) intending edit was achieved at a frequency >25% of good next-generation DNA sequencing (NGS) quality, 2) low heterogeneity in final protein variant generated, and 3) conservation of protein sequence at the target site and/or amino acid residue between cynomolgus genome and human genome (required at amino acid residue being targeted; preferred if 100% conserved). The guides were also screened in CD34+ hematopoietic stem cells (HSCs). Guides identified in the screen and their corresponding edits and maximum editing efficiencies are listed in Table 16. Expression of CD117 in cells edited using the gRNA931 guide (CC200) was at levels comparable to CD117 expression levels in unedited cells (FIG. 18A). Cells edited using gRNA931 lacked binding to ABTx052 (FIG. 18B).

Cells that were base edited using gRNA931 in combination with ABE8.20-NRCH resulted in predominantly the following CD117 variants: S261G; N260D+S261G (˜15%); N260G+S261G (˜3%); and N260S+S261G (˜4%). CD117 variants containing N260S or N260D alterations also contained an S261G alteration, and the combined alterations N260S+S261G or N260D+S261G were observed in a small number of CD117 variants detected (less than 10%).

TABLE 16
Edits corresponding to gRNA samples.

| | Dominant edit of | |
|---|---|---|
| Sample | interest (max %) | Notes |
| gRNA889 | L472P (79.27%) | |
| gRNA908 | S279P (57.6%) | |
| gRNA918 | V64A (56.79%) | |
| gRNA923 | Silent | Near SCF |
| | | binding domain? |
| gRNA928 | S251G (85.17%) | |
| gRNA929 | Silent | Similar to CC128 |
| gRNA931 | S261G (56.06%) | Does not bind to |
| (CC200) | | ABTx052 |
| gRNA944 | Silent | |

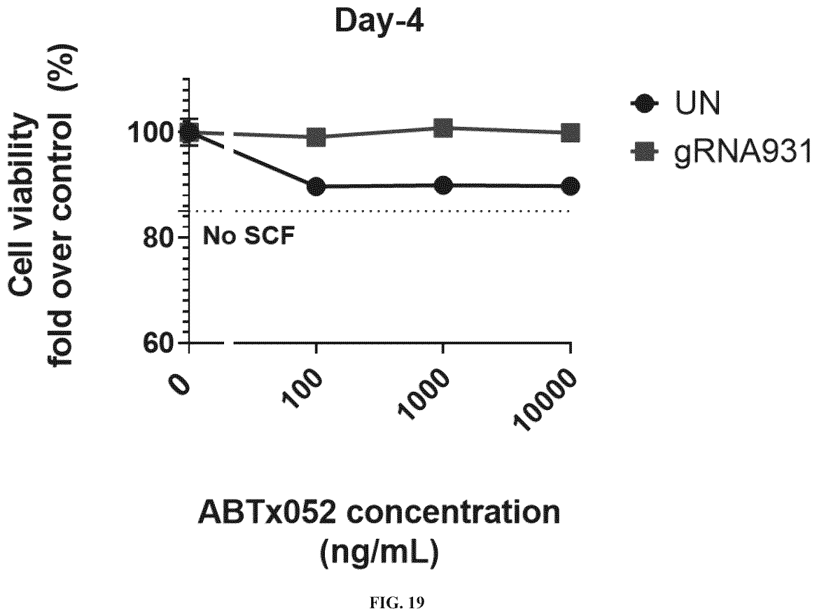

Cells expressing CD117 variants prepared using the gRNA931 guide showed increased viability relative to unedited cells when grown in the presence of ABTx052 at increasing concentrations (FIG. 19). The CD117 S261G engineered epitope was protective against ligand blocking by ABTx052 in vitro. Viability of the edited cells was not affected by addition of ABTx052 at any concentration at the day 4 time point. The viability of unedited cells reached the viability levels of cells cultured without any SCF.

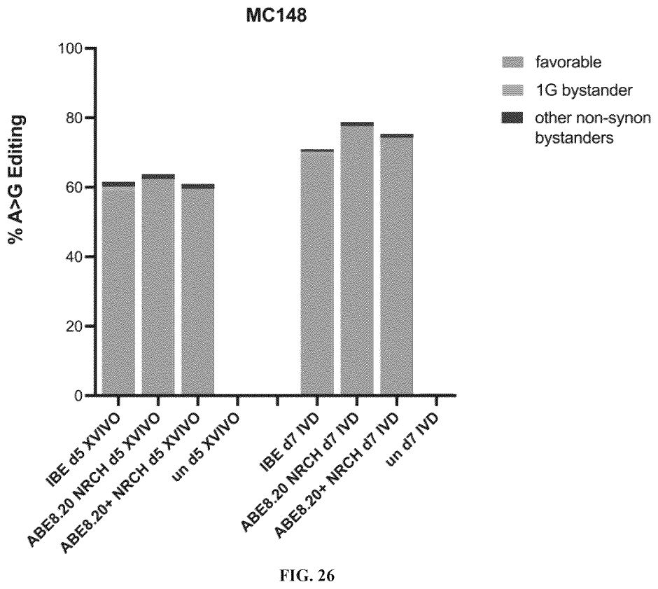

Experiments were undertaking to evaluate the efficiency of base editing of hematopoietic stem cells containing a Sickle Cell Disease mutation (E6V) in the beta hemoglobin gene (HbSS CD34s). The following base editor systems (see FIG. 26) were used: An Inlaid Base Editor (IBE) in combination with sgRNA_017; ABE8.20-NRCH in combination with sgRNA_027; and ABE8.20+ in combination with sgRNA_027. The term “NRCH” refers to the PAM recognized by the Cas9 variant within the indicated base editor, where N represents A, C, G, or T, R represents A or G, and H represents A, C, or T. The amino acid and/or nucleotide sequences for ABE8.20-NRCH, ABE8.20+, and the Inlaid Base Editor (IBE) referenced in FIG. 26 are provided in Table 17 below. The ABE8.20-NRCH base editor contained a TadA*8.20 deaminase domain, the ABE9v1-NRCH base editor contained a TadA*8.20 deaminase domain with the amino acid alteration S82T, and the ABE9v2 base editor contained a TadA*8.20 deaminase domain with the amino acid alterations S82T, Y147D, T166I, and D167N. As shown in FIG. 26, it was found that Makassar editing (i.e., installing the Makassar edit or the Ser9Pro bystander and other non-synonymous bystanders within the target window on the beta globin gene) was achieved at the beta globin locus in the CD34+ cells grown in XVIVO medium (serum-free stem cell medium) 5 days (i.e., “d5”) post electroporation (EP) and 7 days (i.e., “d7”) in in vitro differentiated erythroid cultures (IVD).

TABLE 17
Base editor sequences. In the amino acid sequences, adenosine deaminase
domains are in plain text, linkers are in italics, nucleic acid programmable DNA
binding protein (e.g., Cas9-NRCH or Cas9-MQKFRAER*) domains are in bold, and
bipartite nuclear localization signals are underlined. The Cas9-NRCH domains
recognized the PAM sequence CACC. In embodiments, the ABE8.20-NRCH and
ABE8.20+-NRCH base editors avoid introducing a Ser9Pro bystander edit to a beta
globin gene sequence.

| Editor | Amino Acid or Nucleotide Sequence |
|---|---|
| ABE8.20 | MSEVEFSHEYWMRHALTLAKRARDEREVPVGAVLVLNNRVIGEGWNRAIGLHDPTAH |
| (F149Y)- | AEIMALRQGGLVMQNYRLYDATLYSTFEPCVMCAGAMIHSRIGRVVFGVRNAKTGAA |
| NRCH | GSLMDVLHHPGMNHRVEITEGILADECAALLCRFYRMPRRVFNAQKKAQSSTDSGGS |
| | SGGSSGSETPGTSESATPESSGGSSGGSDKKYSIGLAIGTNSVGWAVITDEYKVPSK |
| | KFKVLGNTDRHSIKKNLIGALLFDSGETAEATRLKRTARRRYTRRKNRICYLQEIFS |
| | NEMAKVDDSFFHRLEESFLVEEDKKHERHPIFGNIVDEVAYHEKYPTIYHLRKKLVD |
| | STDKADLRLIYLALAHMIKFRGHFLIEGDLNPDNSDVDKLFIQLVQTYNQLFEENPI |
| | NASGVDAKAILSARLSKSRRLENLIAQLPGEKKNGLFGNLIALSLGLTPNFKSNFDL |
| | AEDAKLQLSKDTYDDDLDNLLAQIGDQYADLFLAAKNLSDAILLSDILRVNTEITKA |
| | PLSASMVKRYDEHHQDLTLLKALVRQQLPEKYKEIFFDQSKNGYAGYIDGGASQEEF |
| | YKFIKPILEKMDGTEELLVKLNREDLLRKQRTFDNGIIPHQIHLGELHAILRRQGDF |
| | YPFLKDNREKIEKILTFRIPYYVGPLARGNSRFAWMTRKSEETITPWNFEEVVDKGA |
| | SAQSFIERMTNFDKNLPNEKVLPKHSLLYEYFTVYNELTKVKYVTEGMRKPAFLSGE |
| | QKKAIVDLLFKTNRKVTVKQLKEDYFKKIECFDSVEISGVEDRFNASLGTYHDLLKI |
| | IKDKDFLDNEENEDILEDIVLTLTLFEDREMIEERLKTYAHLFDDKVMKQLKRLRYT |
| | GWGRLSRKLINGIRDKQSGKTILDFLKSDGFANRNFMQLIHDDSLTFKEDIQKAQVS |
| | GQGDSLHEHIANLAGSPAIKKGILQTVKVVDELVKVMGGHKPENIVIEMARENQTTQ |
| | KGQKNSRERMKRIEEGIKELGSQILKEHPVENTQLQNEKLYLYYLQNGRDMYVDQEL |
| | DINRLSDYDVDHIVPQSFLKDDSIDNKVLTRSDKNRGKSDNVPSEEVVKKMKNYWRQ |
| | LLNAKLITQRKFDNLTKAERGGLSELDKAGFIKRQLVETRQITKHVAQILDSRMNTK |
| | YDENDKLIREVKVITLKSKLVSDFRKDFQFYKVREINNYHHAHDAYLNAVVGTALIK |
| | KYPKLESEFVYGDYKVYDVRKMIAKSEQEIGKATAKYFFYSNIMNFFKTEITLANGE |
| | IRKRPLIETNGETGEIVWDKGRDFATVRKVLSMPQVNIVKKTEVQTGGFSKESILPK |
| | GNSDKLIARKKDWDPKKYGGFNSPTVAYSVLVVAKVEKGKSKKLKSVKELLGITIME |
| | RSSFEKNPIDFLEAKGYKEVKKDLIIKLPKYSLFELENGRKRMLASAGVLQKGNELA |
| | LPSKYVNFLYLASHYEKLKGSPEDNEQKQLFVEQHKHYLDEIIEQISEFSKRVILAD |
| | ANLDKVLSAYNKHRDKPIREQAENIIHLFTLTNLGAPAAFKYFDTTINRKQYNTTKE |
| | VLDATLIRQSITGLYETRIDLSQLGGDEGADKRTADGSEFESPKKKRKV (SEQ ID |
| | NO: 929) |
| ABE8.20 | TAATACGACTCACTATAAGGAAATAAGAGAGAAAAGAAGAGTAAGAAGAAATATAAG |
| (F149Y)- | AGCCACCATGTCCGAAGTCGAGTTTTCCCATGAGTACTGGATGAGACACGCATTGAC |
| NRCH | TCTCGCAAAGAGGGCTCGAGATGAACGCGAGGTGCCCGTGGGGGCAGTACTCGTGCT |
| | CAACAATCGCGTAATCGGCGAAGGTTGGAATAGGGCAATCGGACTCCACGACCCCAC |
| | TGCACATGCGGAAATCATGGCCCTTCGACAGGGAGGGCTTGTGATGCAGAATTATCG |
| | ACTTTATGATGCGACGCTGTACTCGACGTTTGAACCTTGCGTAATGTGCGCGGGAGC |
| | TATGATTCACTCCCGCATTGGACGAGTTGTATTCGGTGTTCGCAACGCCAAGACGGG |
| | TGCCGCAGGTTCACTGATGGACGTGCTGCATCATCCAGGCATGAACCACCGGGTAGA |
| | AATCACAGAAGGCATATTGGCGGACGAATGTGCGGCGCTGTTGTGTCGTTTTTATCG |
| | CATGCCCAGGCGTGTCTTTAACGCCCAGAAAAAAGCACAATCCTCTACTGACTCCGG |
| | CGGAAGCAGCGGAGGATCTTCTGGAAGCGAAACCCCAGGCACCAGCGAGTCTGCCAC |
| | ACCAGAATCATCTGGCGGTAGCTCCGGCGGCAGCGACAAGAAGTATTCTATCGGACT |
| | GGCCATCGGCACCAACTCTGTTGGATGGGCCGTGATCACCGACGAGTACAAGGTGCC |
| | CAGCAAGAAATTCAAGGTGCTGGGCAACACCGACAGGCACAGCATCAAGAAGAACCT |
| | GATCGGCGCACTGCTGTTCGACTCTGGCGAAACAGCCGAGGCCACCAGACTGAAGAG |
| | AACAGCCCGCAGACGGTACACCAGAAGAAAGAACCGGATCTGCTACCTCCAAGAGAT |
| | CTTCAGCAACGAGATGGCCAAGGTGGACGACAGCTTCTTCCACAGACTGGAAGAGTC |
| | CTTCCTGGTGGAAGAGGACAAGAAGCACGAGAGACACCCCATCTTCGGCAACATCGT |
| | GGACGAGGTGGCCTACCACGAGAAGTACCCCACCATCTACCACCTGAGAAAGAAACT |
| | GGTGGACAGCACCGACAAGGCCGACCTGAGACTGATCTATCTGGCCCTGGCTCACAT |
| | GATCAAGTTCCGGGGCCACTTCCTGATCGAGGGCGACCTGAATCCTGACAACAGCGA |
| | CGTGGACAAGCTGTTCATCCAGCTGGTGCAGACCTACAACCAGCTGTTCGAGGAAAA |
| | CCCCATCAACGCCAGCGGAGTGGATGCCAAGGCCATCCTGTCTGCCAGACTGAGCAA |
| | GAGCAGACGGCTGGAAAATCTGATCGCCCAGCTGCCTGGCGAGAAGAAGAATGGCCT |
| | GTTCGGCAACCTGATTGCCCTGAGCCTGGGCCTGACACCTAACTTCAAGAGCAACTT |
| | CGACCTGGCCGAGGACGCCAAACTGCAGCTGAGCAAGGACACCTACGACGACGACCT |
| | GGACAATCTGCTGGCCCAGATCGGCGATCAGTACGCCGACTTGTTTCTGGCCGCCAA |
| | GAATCTGAGCGACGCCATCCTGCTGTCCGACATCCTGAGAGTGAACACCGAGATCAC |
| | CAAGGCACCTCTGAGCGCCTCTATGGTCAAGAGATACGACGAGCACCACCAGGATCT |
| | GACCCTGCTGAAGGCCCTCGTTAGACAGCAGCTGCCAGAGAAGTACAAAGAGATTTT |
| | CTTCGACCAGAGCAAGAACGGCTACGCCGGCTACATTGATGGCGGAGCCAGCCAAGA |
| | GGAATTCTACAAGTTCATCAAGCCCATCCTCGAGAAGATGGACGGCACCGAGGAACT |
| | GCTGGTCAAGCTGAACAGAGAGGACCTGCTGAGAAAGCAGAGAACCTTCGACAACGG |
| | CATCATCCCTCACCAGATCCACCTGGGAGAACTGCACGCCATTCTGCGGAGACAAGG |
| | GGACTTTTACCCATTCCTGAAGGACAACCGGGAAAAGATCGAGAAAATCCTGACCTT |
| | CAGGATCCCCTACTACGTGGGACCACTGGCCAGAGGCAATAGCAGATTCGCCTGGAT |
| | GACCAGAAAGAGCGAGGAAACCATCACTCCCTGGAACTTCGAGGAAGTGGTGGACAA |
| | GGGCGCCAGCGCTCAGTCCTTCATCGAGCGGATGACCAACTTCGATAAGAACCTGCC |
| | TAACGAGAAGGTGCTGCCCAAGCACAGCCTGCTGTACGAGTACTTCACCGTGTACAA |
| | CGAGCTGACCAAAGTGAAATACGTGACCGAGGGAATGAGAAAGCCCGCCTTTCTGAG |
| | CGGCGAGCAGAAAAAGGCCATCGTGGATCTGCTGTTCAAGACCAACCGGAAAGTGAC |
| | CGTGAAGCAGCTGAAAGAGGACTACTTCAAGAAAATCGAGTGCTTCGACAGCGTCGA |
| | GATCTCCGGCGTGGAAGATCGGTTCAATGCCAGCCTGGGCACATACCACGATCTGCT |
| | GAAAATTATCAAGGACAAGGACTTCCTGGACAACGAAGAGAACGAGGACATCCTTGA |
| | GGACATCGTGCTGACACTGACCCTGTTTGAGGACAGAGAGATGATCGAGGAACGGCT |
| | GAAAACATACGCCCACCTGTTCGACGACAAAGTGATGAAGCAACTGAAGCGGCTGAG |
| | ATACACCGGCTGGGGCAGACTGTCTCGGAAGCTGATCAACGGCATCCGGGATAAGCA |
| | GTCCGGCAAGACCATCCTGGACTTTCTGAAGTCCGACGGCTTCGCCAACAGAAACTT |
| | CATGCAGCTGATTCACGACGACAGCCTCACCTTCAAAGAGGATATCCAGAAAGCCCA |
| | GGTGTCCGGCCAGGGCGATTCTCTGCATGAGCACATTGCCAACCTGGCCGGCTCTCC |
| | CGCCATTAAGAAAGGCATCCTGCAGACAGTGAAGGTGGTGGACGAGCTTGTGAAAGT |
| | GATGGGCGGACACAAGCCCGAGAACATCGTGATCGAAATGGCCAGAGAGAACCAGAC |
| | CACACAGAAGGGACAGAAGAACAGCCGCGAGAGAATGAAGCGGATCGAAGAGGGCAT |
| | CAAAGAGCTGGGCAGCCAGATCCTGAAAGAACACCCCGTGGAAAACACCCAGCTGCA |
| | GAACGAGAAGCTGTACCTGTACTACCTGCAGAATGGACGGGATATGTACGTGGACCA |
| | AGAGCTGGACATCAACAGACTGTCCGACTACGATGTGGACCATATCGTGCCCCAGTC |
| | TTTTCTGAAGGACGACTCCATCGACAACAAGGTCCTGACCAGATCCGACAAGAATCG |
| | GGGCAAGAGCGACAACGTGCCCTCCGAAGAGGTGGTCAAGAAGATGAAGAACTACTG |
| | GCGACAGCTGCTGAACGCCAAGCTGATTACCCAGCGGAAGTTCGACAATCTGACCAA |
| | GGCCGAAAGAGGCGGCCTGAGCGAACTGGATAAGGCCGGCTTCATCAAGAGACAGCT |
| | GGTGGAAACCCGGCAGATCACAAAGCACGTGGCACAGATTCTGGACTCTCGGATGAA |
| | CACTAAGTACGACGAGAACGACAAACTGATCCGCGAAGTGAAAGTCATCACCCTGAA |
| | GTCCAAGCTGGTGTCCGATTTCCGGAAGGATTTCCAGTTCTACAAAGTGCGCGAGAT |
| | CAACAACTACCATCACGCCCACGACGCCTACCTGAATGCCGTTGTTGGAACAGCCCT |
| | GATCAAAAAGTACCCTAAGCTGGAAAGCGAGTTCGTGTACGGCGACTACAAGGTGTA |
| | CGACGTGCGGAAGATGATCGCCAAGAGCGAGCAAGAGATTGGCAAGGCAACCGCCAA |
| | GTACTTCTTCTACAGCAACATCATGAACTTTTTCAAGACAGAGATCACCCTCGCCAA |
| | CGGCGAGATCAGAAAGCGGCCTCTGATCGAGACAAACGGCGAAACCGGCGAGATTGT |
| | GTGGGATAAGGGCAGAGACTTTGCCACAGTGCGGAAAGTGCTGAGCATGCCCCAAGT |
| | GAATATCGTGAAGAAAACCGAGGTGCAGACAGGCGGCTTCAGCAAAGAGTCTATCCT |
| | GCCTAAGGGGAACTCCGACAAGCTGATCGCCAGAAAGAAGGACTGGGACCCCAAGAA |
| | ATACGGCGGCTTTAACTCTCCCACCGTGGCCTATTCTGTTCTGGTGGTGGCCAAAGT |
| | GGAAAAGGGCAAGTCCAAGAAACTCAAGAGCGTGAAAGAGCTGCTGGGGATCACCAT |
| | CATGGAAAGAAGCAGCTTCGAGAAGAATCCGATCGATTTCCTCGAGGCCAAGGGTTA |
| | CAAAGAAGTGAAAAAGGACCTGATCATCAAGCTCCCCAAGTACTCCCTGTTCGAGCT |
| | GGAAAACGGCCGGAAGAGAATGCTGGCCTCTGCCGGTGTCCTGCAGAAGGGAAACGA |
| | ACTGGCCCTGCCTAGCAAATATGTGAACTTCCTGTACCTGGCCAGCCACTATGAGAA |
| | GCTGAAGGGCAGCCCCGAGGACAATGAGCAAAAGCAGCTGTTTGTGGAACAGCACAA |
| | GCACTACCTGGACGAGATCATCGAGCAGATCAGCGAGTTTAGCAAGAGAGTGATTCT |
| | GGCCGACGCCAATCTGGACAAAGTGCTGTCCGCCTACAACAAGCACCGGGACAAGCC |
| | TATCAGAGAGCAGGCCGAGAATATCATCCACCTGTTTACCCTGACCAACCTGGGAGC |
| | CCCTGCGGCCTTCAAGTACTTTGACACCACCATCAACAGGAAGCAGTACAACACCAC |
| | CAAAGAGGTGCTGGACGCCACTCTGATCCGCCAGTCTATCACCGGCCTGTACGAGAC |
| | ACGGATCGACCTGTCTCAACTCGGAGGCGACGAAGGCGCCGATAAGAGAACCGCCGA |
| | TGGCTCTGAGTTCGAGAGCCCTAAGAAAAAGCGCAAAGTGTAG (SEQ ID NO: 930) |
| ABE8.20+ | MSEVEFSHEYWMRHALTLAKRARDEREVPVGAVLVLNNRVIGEGWNRAIGLHDPTAH |
| (ABE8.20 | AEIMALRQGGLVMQNYRLYDATLYSTFEPCVMCAGAMIHSRIGRVVFGVRNAKTGAA |
| w/ the | GSLMDVLHHPGMNHRVEITEGILADECAALLCDFYRMPRRVFNAQKKAQSSINSGGS |
| alterations | SGGSSGSETPGTSESATPESSGGSSGGSDKKYSIGLAIGTNSVGWAVITDEYKVPSK |
| R147D, | KFKVLGNTDRHSIKKNLIGALLFDSGETAEATRLKRTARRRYTRRKNRICYLQEIFS |
| F149Y, | NEMAKVDDSFFHRLEESFLVEEDKKHERHPIFGNIVDEVAYHEKYPTIYHLRKKLVD |
| T166I, and | STDKADLRLIYLALAHMIKFRGHFLIEGDLNPDNSDVDKLFIQLVQTYNQLFEENPI |
| D167N in | NASGVDAKAILSARLSKSRRLENLIAQLPGEKKNGLFGNLIALSLGLTPNFKSNFDL |
| the TadA* | AEDAKLQLSKDTYDDDLDNLLAQIGDQYADLFLAAKNLSDAILLSDILRVNTEITKA |
| domain) | PLSASMVKRYDEHHQDLTLLKALVRQQLPEKYKEIFFDQSKNGYAGYIDGGASQEEF |
| | YKFIKPILEKMDGTEELLVKLNREDLLRKQRTFDNGIIPHQIHLGELHAILRRQGDF |
| | YPFLKDNREKIEKILTFRIPYYVGPLARGNSRFAWMTRKSEETITPWNFEEVVDKGA |
| | SAQSFIERMTNFDKNLPNEKVLPKHSLLYEYFTVYNELTKVKYVTEGMRKPAFLSGE |
| | QKKAIVDLLFKTNRKVTVKQLKEDYFKKIECFDSVEISGVEDRFNASLGTYHDLLKI |
| | IKDKDFLDNEENEDILEDIVLTLTLFEDREMIEERLKTYAHLFDDKVMKQLKRLRYT |
| | GWGRLSRKLINGIRDKQSGKTILDFLKSDGFANRNFMQLIHDDSLTFKEDIQKAQVS |
| | GQGDSLHEHIANLAGSPAIKKGILQTVKVVDELVKVMGGHKPENIVIEMARENQTTQ |
| | KGQKNSRERMKRIEEGIKELGSQILKEHPVENTQLQNEKLYLYYLQNGRDMYVDQEL |
| | DINRLSDYDVDHIVPQSFLKDDSIDNKVLTRSDKNRGKSDNVPSEEVVKKMKNYWRQ |
| | LLNAKLITQRKFDNLTKAERGGLSELDKAGFIKRQLVETRQITKHVAQILDSRMNTK |
| | YDENDKLIREVKVITLKSKLVSDFRKDFQFYKVREINNYHHAHDAYLNAVVGTALIK |
| | KYPKLESEFVYGDYKVYDVRKMIAKSEQEIGKATAKYFFYSNIMNFFKTEITLANGE |
| | IRKRPLIETNGETGEIVWDKGRDFATVRKVLSMPQVNIVKKTEVQTGGFSKESILPK |
| | GNSDKLIARKKDWDPKKYGGFNSPTVAYSVLVVAKVEKGKSKKLKSVKELLGITIME |
| | RSSFEKNPIDFLEAKGYKEVKKDLIIKLPKYSLFELENGRKRMLASAGVLQKGNELA |
| | LPSKYVNFLYLASHYEKLKGSPEDNEQKQLFVEQHKHYLDEIIEQISEFSKRVILAD |
| | ANLDKVLSAYNKHRDKPIREQAENIIHLFTLTNLGAPAAFKYFDTTINRKQYNTTKE |
| | VLDATLIRQSITGLYETRIDLSQLGGDEGADKRTADGSEFESPKKKRKV (SEQ ID |
| | NO: 931) |
| ABE8.20+ | TAATACGACTCACTATAAGGAAATAAGAGAGAAAAGAAGAGTAAGAAGAAATATAAG |
| | AGCCACCATGTCCGAAGTCGAGTTTTCCCATGAGTACTGGATGAGACACGCATTGAC |
| | TCTCGCAAAGAGGGCTCGAGATGAACGCGAGGTGCCCGTGGGGGCAGTACTCGTGCT |
| | CAACAATCGCGTAATCGGCGAAGGTTGGAATAGGGCAATCGGACTCCACGACCCCAC |
| | TGCACATGCGGAAATCATGGCCCTTCGACAGGGAGGGCTTGTGATGCAGAATTATCG |
| | ACTTTATGATGCGACGCTGTACTCGACGTTTGAACCTTGCGTAATGTGCGCGGGAGC |
| | TATGATTCACTCCCGCATTGGACGAGTTGTATTCGGTGTTCGCAACGCCAAGACGGG |
| | TGCCGCAGGTTCACTGATGGACGTGCTGCATCATCCAGGCATGAACCACCGGGTAGA |
| | AATCACAGAAGGCATATTGGCGGACGAATGTGCGGCGCTGTTGTGTGACTTTTATCG |
| | CATGCCCAGGCGTGTCTTTAACGCCCAGAAAAAAGCACAATCCTCTATAAACTCCGG |
| | CGGAAGCAGCGGAGGATCTTCTGGAAGCGAAACCCCAGGCACCAGCGAGTCTGCCAC |
| | ACCAGAATCATCTGGCGGTAGCTCCGGCGGCAGCGACAAGAAGTATTCTATCGGACT |
| | GGCCATCGGCACCAACTCTGTTGGATGGGCCGTGATCACCGACGAGTACAAGGTGCC |
| | CAGCAAGAAATTCAAGGTGCTGGGCAACACCGACAGGCACAGCATCAAGAAGAACCT |
| | GATCGGCGCACTGCTGTTCGACTCTGGCGAAACAGCCGAGGCCACCAGACTGAAGAG |
| | AACAGCCCGCAGACGGTACACCAGAAGAAAGAACCGGATCTGCTACCTCCAAGAGAT |
| | CTTCAGCAACGAGATGGCCAAGGTGGACGACAGCTTCTTCCACAGACTGGAAGAGTC |
| | CTTCCTGGTGGAAGAGGACAAGAAGCACGAGAGACACCCCATCTTCGGCAACATCGT |
| | GGACGAGGTGGCCTACCACGAGAAGTACCCCACCATCTACCACCTGAGAAAGAAACT |
| | GGTGGACAGCACCGACAAGGCCGACCTGAGACTGATCTATCTGGCCCTGGCTCACAT |
| | GATCAAGTTCCGGGGCCACTTCCTGATCGAGGGCGACCTGAATCCTGACAACAGCGA |
| | CGTGGACAAGCTGTTCATCCAGCTGGTGCAGACCTACAACCAGCTGTTCGAGGAAAA |
| | CCCCATCAACGCCAGCGGAGTGGATGCCAAGGCCATCCTGTCTGCCAGACTGAGCAA |
| | GAGCAGACGGCTGGAAAATCTGATCGCCCAGCTGCCTGGCGAGAAGAAGAATGGCCT |
| | GTTCGGCAACCTGATTGCCCTGAGCCTGGGCCTGACACCTAACTTCAAGAGCAACTT |
| | CGACCTGGCCGAGGACGCCAAACTGCAGCTGAGCAAGGACACCTACGACGACGACCT |
| | GGACAATCTGCTGGCCCAGATCGGCGATCAGTACGCCGACTTGTTTCTGGCCGCCAA |
| | GAATCTGAGCGACGCCATCCTGCTGTCCGACATCCTGAGAGTGAACACCGAGATCAC |
| | CAAGGCACCTCTGAGCGCCTCTATGGTCAAGAGATACGACGAGCACCACCAGGATCT |
| | GACCCTGCTGAAGGCCCTCGTTAGACAGCAGCTGCCAGAGAAGTACAAAGAGATTTT |
| | CTTCGACCAGAGCAAGAACGGCTACGCCGGCTACATTGATGGCGGAGCCAGCCAAGA |
| | GGAATTCTACAAGTTCATCAAGCCCATCCTCGAGAAGATGGACGGCACCGAGGAACT |
| | GCTGGTCAAGCTGAACAGAGAGGACCTGCTGAGAAAGCAGAGAACCTTCGACAACGG |
| | CATCATCCCTCACCAGATCCACCTGGGAGAACTGCACGCCATTCTGCGGAGACAAGG |
| | GGACTTTTACCCATTCCTGAAGGACAACCGGGAAAAGATCGAGAAAATCCTGACCTT |
| | CAGGATCCCCTACTACGTGGGACCACTGGCCAGAGGCAATAGCAGATTCGCCTGGAT |
| | GACCAGAAAGAGCGAGGAAACCATCACTCCCTGGAACTTCGAGGAAGTGGTGGACAA |
| | GGGCGCCAGCGCTCAGTCCTTCATCGAGCGGATGACCAACTTCGATAAGAACCTGCC |
| | TAACGAGAAGGTGCTGCCCAAGCACAGCCTGCTGTACGAGTACTTCACCGTGTACAA |
| | CGAGCTGACCAAAGTGAAATACGTGACCGAGGGAATGAGAAAGCCCGCCTTTCTGAG |
| | CGGCGAGCAGAAAAAGGCCATCGTGGATCTGCTGTTCAAGACCAACCGGAAAGTGAC |
| | CGTGAAGCAGCTGAAAGAGGACTACTTCAAGAAAATCGAGTGCTTCGACAGCGTCGA |
| | GATCTCCGGCGTGGAAGATCGGTTCAATGCCAGCCTGGGCACATACCACGATCTGCT |
| | GAAAATTATCAAGGACAAGGACTTCCTGGACAACGAAGAGAACGAGGACATCCTTGA |
| | GGACATCGTGCTGACACTGACCCTGTTTGAGGACAGAGAGATGATCGAGGAACGGCT |
| | GAAAACATACGCCCACCTGTTCGACGACAAAGTGATGAAGCAACTGAAGCGGCTGAG |
| | ATACACCGGCTGGGGCAGACTGTCTCGGAAGCTGATCAACGGCATCCGGGATAAGCA |
| | GTCCGGCAAGACCATCCTGGACTTTCTGAAGTCCGACGGCTTCGCCAACAGAAACTT |
| | CATGCAGCTGATTCACGACGACAGCCTCACCTTCAAAGAGGATATCCAGAAAGCCCA |
| | GGTGTCCGGCCAGGGCGATTCTCTGCATGAGCACATTGCCAACCTGGCCGGCTCTCC |
| | CGCCATTAAGAAAGGCATCCTGCAGACAGTGAAGGTGGTGGACGAGCTTGTGAAAGT |
| | GATGGGCGGACACAAGCCCGAGAACATCGTGATCGAAATGGCCAGAGAGAACCAGAC |
| | CACACAGAAGGGACAGAAGAACAGCCGCGAGAGAATGAAGCGGATCGAAGAGGGCAT |
| | CAAAGAGCTGGGCAGCCAGATCCTGAAAGAACACCCCGTGGAAAACACCCAGCTGCA |
| | GAACGAGAAGCTGTACCTGTACTACCTGCAGAATGGACGGGATATGTACGTGGACCA |
| | AGAGCTGGACATCAACAGACTGTCCGACTACGATGTGGACCATATCGTGCCCCAGTC |
| | TTTTCTGAAGGACGACTCCATCGACAACAAGGTCCTGACCAGATCCGACAAGAATCG |
| | GGGCAAGAGCGACAACGTGCCCTCCGAAGAGGTGGTCAAGAAGATGAAGAACTACTG |
| | GCGACAGCTGCTGAACGCCAAGCTGATTACCCAGCGGAAGTTCGACAATCTGACCAA |
| | GGCCGAAAGAGGCGGCCTGAGCGAACTGGATAAGGCCGGCTTCATCAAGAGACAGCT |
| | GGTGGAAACCCGGCAGATCACAAAGCACGTGGCACAGATTCTGGACTCTCGGATGAA |
| | CACTAAGTACGACGAGAACGACAAACTGATCCGCGAAGTGAAAGTCATCACCCTGAA |
| | GTCCAAGCTGGTGTCCGATTTCCGGAAGGATTTCCAGTTCTACAAAGTGCGCGAGAT |
| | CAACAACTACCATCACGCCCACGACGCCTACCTGAATGCCGTTGTTGGAACAGCCCT |
| | GATCAAAAAGTACCCTAAGCTGGAAAGCGAGTTCGTGTACGGCGACTACAAGGTGTA |
| | CGACGTGCGGAAGATGATCGCCAAGAGCGAGCAAGAGATTGGCAAGGCAACCGCCAA |
| | GTACTTCTTCTACAGCAACATCATGAACTTTTTCAAGACAGAGATCACCCTCGCCAA |
| | CGGCGAGATCAGAAAGCGGCCTCTGATCGAGACAAACGGCGAAACCGGCGAGATTGT |
| | GTGGGATAAGGGCAGAGACTTTGCCACAGTGCGGAAAGTGCTGAGCATGCCCCAAGT |
| | GAATATCGTGAAGAAAACCGAGGTGCAGACAGGCGGCTTCAGCAAAGAGTCTATCCT |
| | GCCTAAGGGGAACTCCGACAAGCTGATCGCCAGAAAGAAGGACTGGGACCCCAAGAA |
| | ATACGGCGGCTTTAACTCTCCCACCGTGGCCTATTCTGTTCTGGTGGTGGCCAAAGT |
| | GGAAAAGGGCAAGTCCAAGAAACTCAAGAGCGTGAAAGAGCTGCTGGGGATCACCAT |
| | CATGGAAAGAAGCAGCTTCGAGAAGAATCCGATCGATTTCCTCGAGGCCAAGGGTTA |
| | CAAAGAAGTGAAAAAGGACCTGATCATCAAGCTCCCCAAGTACTCCCTGTTCGAGCT |
| | GGAAAACGGCCGGAAGAGAATGCTGGCCTCTGCCGGTGTCCTGCAGAAGGGAAACGA |
| | ACTGGCCCTGCCTAGCAAATATGTGAACTTCCTGTACCTGGCCAGCCACTATGAGAA |
| | GCTGAAGGGCAGCCCCGAGGACAATGAGCAAAAGCAGCTGTTTGTGGAACAGCACAA |
| | GCACTACCTGGACGAGATCATCGAGCAGATCAGCGAGTTTAGCAAGAGAGTGATTCT |
| | GGCCGACGCCAATCTGGACAAAGTGCTGTCCGCCTACAACAAGCACCGGGACAAGCC |
| | TATCAGAGAGCAGGCCGAGAATATCATCCACCTGTTTACCCTGACCAACCTGGGAGC |
| | CCCTGCGGCCTTCAAGTACTTTGACACCACCATCAACAGGAAGCAGTACAACACCAC |
| | CAAAGAGGTGCTGGACGCCACTCTGATCCGCCAGTCTATCACCGGCCTGTACGAGAC |
| | ACGGATCGACCTGTCTCAACTCGGAGGCGACGAAGGCGCCGATAAGAGAACCGCCGA |
| | TGGCTCTGAGTTCGAGAGCCCTAAGAAAAAGCGCAAAGTGTAG (SEQ ID NO: 932) |
| Inlaid Base | MDKKYSIGLAIGTNSVGWAVITDEYKVPSKKFKVLGNTDRHSIKKNLIGALLFDSGE |
| Editor | TAEATRLKRTARRRYTRRKNRICYLQEIFSNEMAKVDDSFFHRLEESFLVEEDKKHE |
| (IBE) | RHPIFGNIVDEVAYHEKYPTIYHLRKKLVDSTDKADLRLIYLALAHMIKFRGHFLIE |
| referenced | GDLNPDNSDVDKLFIQLVQTYNQLFEENPINASGVDAKAILSARLSKSRRLENLIAQ |
| in FIG. 26 | LPGEKKNGLFGNLIALSLGLTPNFKSNFDLAEDAKLQLSKDTYDDDLDNLLAQIGDQ |
| (MSP684 | YADLFLAAKNLSDAILLSDILRVNTEITKAPLSASMIKRYDEHHQDLTLLKALVRQQ |
| mono TadA- | LPEKYKEIFFDQSKNGYAGYIDGGASQEEFYKFIKPILEKMDGTEELLVKLNREDLL |
| TadA7.10 | RKQRTFDNGSIPHQIHLGELHAILRRQEDFYPFLKDNREKIEKILTFRIPYYVGPLA |
| (V82S)- | RGNSRFAWMTRKSEETITPWNFEEVVDKGASAQSFIERMTNFDKNLPNEKVLPKHSL |
| MQKFRAER) | LYEYFTVYNELTKVKYVTEGMRKPAFLSGEQKKAIVDLLFKTNRKVTVKQLKEDYFK |
| | KIECFDSVEISGVEDRFNASLGTYHDLLKIIKDKDFLDNEENEDILEDIVLTLTLFE |
| | DREMIEERLKTYAHLFDDKVMKQLKRRRYTGWGRLSRKLINGIRDKQSGKTILDFLK |
| | SDGFANRNFMQLIHDDSLTFKEDIQKAQVSGQGDSLHEHIANLAGSPAIKKGILQTV |
| | KVVDELVKVMGRHKPENIVIEMARENQTTQKGQKNSRERMKRIEEGIKELGSQILKE |
| | HPVENTQLQNEKLYLYYLQNGRDMYVDQELDINRLSDYDVDHIVPQSFLKDDSIDNK |
| | VLTRSDKNRGKSDNVPSEEVVKKMKNYWRQLLNAKLITQRKFDNLTKAERGGLSELD |
| | KAGFIKRQLVETRQITKHVAQILDSRMNTKYDENDKLIREVKVITLKSKLVSDFRKD |
| | FQFYKVREINNYHHAHDAYLNAVVGTALIKKYPKLESEFVYGDYKVYDVRKMIAKSE |
| | QEIGSSGSETPGTSESATPESSGSEVEFSHEYWMRHALTLAKRARDEREVPVGAVLV |
| | LNNRVIGEGWNRAIGLHDPTAHAEIMALRQGGLVMQNYRLIDATLYSTFEPCVMCAG |
| | AMIHSRIGRVVFGVRNAKTGAAGSLMDVLHYPGMNHRVEITEGILADECAALLCYFF |
| | RMPRQVFNAQKKAQSSTDGKATAKYFFYSNIMNFFKTEITLANGEIRKRPLIETNGE |
| | TGEIVWDKGRDFATVRKVLSMPQVNIVKKTEVQTGGFSKESILPKRNSDKLIARKKD |
| | WDPKKYGGFMQPTVAYSVLVVAKVEKGKSKKLKSVKELLGITIMERSSFEKNPIDFL |
| | EAKGYKEVKKDLIIKLPKYSLFELENGRKRMLASAKFLQKGNELALPSKYVNFLYLA |
| | SHYEKLKGSPEDNEQKQLFVEQHKHYLDEIIEQISEFSKRVILADANLDKVLSAYNK |
| | HRDKPIREQAENIIHLFTLTNLGAPRAFKYFDTTIARKEYRSTKEVLDATLIHQSIT |
| | GLYETRIDLSQLGGD (SEQ ID NO: 933) |
| ABE9v1- | MSEVEFSHEYWMRHALTLAKRARDEREVPVGAVLVLNNRVIGEGWNRAIGLHDPTAH |
| NRCH | AEIMALRQGGLVMQNYRLYDATLYTTFEPCVMCAGAMIHSRIGRVVFGVRNAKTGAA |
| (ABE8.20 | GSLMDVLHHPGMNHRVEITEGILADECAALLCRFYRMPRRVFNAQKKAQSSTDSGGS |
| w/ the | SGGSSGSETPGTSESATPESSGGSSGGSDKKYSIGLAIGTNSVGWAVITDEYKVPSK |
| alterations | KFKVLGNTDRHSIKKNLIGALLFDSGETAEATRLKRTARRRYTRRKNRICYLQEIFS |
| S82T and | NEMAKVDDSFFHRLEESFLVEEDKKHERHPIFGNIVDEVAYHEKYPTIYHLRKKLVD |
| F149Y in | STDKADLRLIYLALAHMIKFRGHFLIEGDLNPDNSDVDKLFIQLVQTYNQLFEENPI |
| the TadA* | NASGVDAKAILSARLSKSRRLENLIAQLPGEKKNGLFGNLIALSLGLTPNFKSNFDL |
| domain) | AEDAKLQLSKDTYDDDLDNLLAQIGDQYADLFLAAKNLSDAILLSDILRVNTEITKA |
| | PLSASMVKRYDEHHQDLTLLKALVRQQLPEKYKEIFFDQSKNGYAGYIDGGASQEEF |
| | YKFIKPILEKMDGTEELLVKLNREDLLRKQRTFDNGIIPHQIHLGELHAILRRQGDF |
| | YPFLKDNREKIEKILTFRIPYYVGPLARGNSRFAWMTRKSEETITPWNFEEVVDKGA |
| | SAQSFIERMTNFDKNLPNEKVLPKHSLLYEYFTVYNELTKVKYVTEGMRKPAFLSGE |
| | QKKAIVDLLFKTNRKVTVKQLKEDYFKKIECFDSVEISGVEDRFNASLGTYHDLLKI |
| | IKDKDFLDNEENEDILEDIVLTLTLFEDREMIEERLKTYAHLFDDKVMKQLKRLRYT |
| | GWGRLSRKLINGIRDKQSGKTILDFLKSDGFANRNFMQLIHDDSLTFKEDIQKAQVS |
| | GQGDSLHEHIANLAGSPAIKKGILQTVKVVDELVKVMGGHKPENIVIEMARENQTTQ |
| | KGQKNSRERMKRIEEGIKELGSQILKEHPVENTQLQNEKLYLYYLQNGRDMYVDQEL |
| | DINRLSDYDVDHIVPQSFLKDDSIDNKVLTRSDKNRGKSDNVPSEEVVKKMKNYWRQ |
| | LLNAKLITQRKFDNLTKAERGGLSELDKAGFIKRQLVETRQITKHVAQILDSRMNTK |
| | YDENDKLIREVKVITLKSKLVSDFRKDFQFYKVREINNYHHAHDAYLNAVVGTALIK |
| | KYPKLESEFVYGDYKVYDVRKMIAKSEQEIGKATAKYFFYSNIMNFFKTEITLANGE |
| | IRKRPLIETNGETGEIVWDKGRDFATVRKVLSMPQVNIVKKTEVQTGGFSKESILPK |
| | GNSDKLIARKKDWDPKKYGGFNSPTVAYSVLVVAKVEKGKSKKLKSVKELLGITIME |
| | RSSFEKNPIDFLEAKGYKEVKKDLIIKLPKYSLFELENGRKRMLASAGVLQKGNELA |
| | LPSKYVNFLYLASHYEKLKGSPEDNEQKQLFVEQHKHYLDEIIEQISEFSKRVILAD |
| | ANLDKVLSAYNKHRDKPIREQAENIIHLFTLTNLGAPAAFKYFDTTINRKQYNTTKE |
| | VLDATLIRQSITGLYETRIDLSQLGGDEGADKRTADGSEFESPKKKRKV (SEQ ID |
| | NO: 934) |
| ABE9v1- | ATGTCCGAAGTCGAGTTTTCCCATGAGTACTGGATGAGACACGCATTGACTCTCGCA |
| NRCH | AAGAGGGCTCGAGATGAACGCGAGGTGCCCGTGGGGGCAGTACTCGTGCTCAACAAT |
| | CGCGTAATCGGCGAAGGTTGGAATAGGGCAATCGGACTCCACGACCCCACTGCACAT |
| | GCGGAAATCATGGCCCTTCGACAGGGAGGGCTTGTGATGCAGAATTATCGACTTTAT |
| | GATGCGACGCTGTACACCACGTTTGAACCTTGCGTAATGTGCGCGGGAGCTATGATT |
| | CACTCCCGCATTGGACGAGTTGTATTCGGTGTTCGCAACGCCAAGACGGGTGCCGCA |
| | GGTTCACTGATGGACGTGCTGCATCATCCAGGCATGAACCACCGGGTAGAAATCACA |
| | GAAGGCATATTGGCGGACGAATGTGCGGCGCTGTTGTGTCGTTTTTATCGCATGCCC |
| | AGGCGTGTCTTTAACGCCCAGAAAAAAGCACAATCCTCTACTGACTCCGGCGGAAGC |
| | AGCGGAGGATCTTCTGGAAGCGAAACCCCAGGCACCAGCGAGTCTGCCACACCAGAA |
| | TCATCTGGCGGTAGCTCCGGCGGCAGCGACAAGAAGTATTCTATCGGACTGGCCATC |
| | GGCACCAACTCTGTTGGATGGGCCGTGATCACCGACGAGTACAAGGTGCCCAGCAAG |
| | AAATTCAAGGTGCTGGGCAACACCGACAGGCACAGCATCAAGAAGAACCTGATCGGC |
| | GCACTGCTGTTCGACTCTGGCGAAACAGCCGAGGCCACCAGACTGAAGAGAACAGCC |
| | CGCAGACGGTACACCAGAAGAAAGAACCGGATCTGCTACCTCCAAGAGATCTTCAGC |
| | AACGAGATGGCCAAGGTGGACGACAGCTTCTTCCACAGACTGGAAGAGTCCTTCCTG |
| | GTGGAAGAGGACAAGAAGCACGAGAGACACCCCATCTTCGGCAACATCGTGGACGAG |
| | GTGGCCTACCACGAGAAGTACCCCACCATCTACCACCTGAGAAAGAAACTGGTGGAC |
| | AGCACCGACAAGGCCGACCTGAGACTGATCTATCTGGCCCTGGCTCACATGATCAAG |
| | TTCCGGGGCCACTTCCTGATCGAGGGCGACCTGAATCCTGACAACAGCGACGTGGAC |
| | AAGCTGTTCATCCAGCTGGTGCAGACCTACAACCAGCTGTTCGAGGAAAACCCCATC |
| | AACGCCAGCGGAGTGGATGCCAAGGCCATCCTGTCTGCCAGACTGAGCAAGAGCAGA |
| | CGGCTGGAAAATCTGATCGCCCAGCTGCCTGGCGAGAAGAAGAATGGCCTGTTCGGC |
| | AACCTGATTGCCCTGAGCCTGGGCCTGACACCTAACTTCAAGAGCAACTTCGACCTG |
| | GCCGAGGACGCCAAACTGCAGCTGAGCAAGGACACCTACGACGACGACCTGGACAAT |
| | CTGCTGGCCCAGATCGGCGATCAGTACGCCGACTTGTTTCTGGCCGCCAAGAATCTG |
| | AGCGACGCCATCCTGCTGTCCGACATCCTGAGAGTGAACACCGAGATCACCAAGGCA |
| | CCTCTGAGCGCCTCTATGGTCAAGAGATACGACGAGCACCACCAGGATCTGACCCTG |
| | CTGAAGGCCCTCGTTAGACAGCAGCTGCCAGAGAAGTACAAAGAGATTTTCTTCGAC |
| | CAGAGCAAGAACGGCTACGCCGGCTACATTGATGGCGGAGCCAGCCAAGAGGAATTC |
| | TACAAGTTCATCAAGCCCATCCTCGAGAAGATGGACGGCACCGAGGAACTGCTGGTC |
| | AAGCTGAACAGAGAGGACCTGCTGAGAAAGCAGAGAACCTTCGACAACGGCATCATC |
| | CCTCACCAGATCCACCTGGGAGAACTGCACGCCATTCTGCGGAGACAAGGGGACTTT |
| | TACCCATTCCTGAAGGACAACCGGGAAAAGATCGAGAAAATCCTGACCTTCAGGATC |
| | CCCTACTACGTGGGACCACTGGCCAGAGGCAATAGCAGATTCGCCTGGATGACCAGA |
| | AAGAGCGAGGAAACCATCACTCCCTGGAACTTCGAGGAAGTGGTGGACAAGGGCGCC |
| | AGCGCTCAGTCCTTCATCGAGCGGATGACCAACTTCGATAAGAACCTGCCTAACGAG |
| | AAGGTGCTGCCCAAGCACAGCCTGCTGTACGAGTACTTCACCGTGTACAACGAGCTG |
| | ACCAAAGTGAAATACGTGACCGAGGGAATGAGAAAGCCCGCCTTTCTGAGCGGCGAG |
| | CAGAAAAAGGCCATCGTGGATCTGCTGTTCAAGACCAACCGGAAAGTGACCGTGAAG |
| | CAGCTGAAAGAGGACTACTTCAAGAAAATCGAGTGCTTCGACAGCGTCGAGATCTCC |
| | GGCGTGGAAGATCGGTTCAATGCCAGCCTGGGCACATACCACGATCTGCTGAAAATT |
| | ATCAAGGACAAGGACTTCCTGGACAACGAAGAGAACGAGGACATCCTTGAGGACATC |
| | GTGCTGACACTGACCCTGTTTGAGGACAGAGAGATGATCGAGGAACGGCTGAAAACA |
| | TACGCCCACCTGTTCGACGACAAAGTGATGAAGCAACTGAAGCGGCTGAGATACACC |
| | GGCTGGGGCAGACTGTCTCGGAAGCTGATCAACGGCATCCGGGATAAGCAGTCCGGC |
| | AAGACCATCCTGGACTTTCTGAAGTCCGACGGCTTCGCCAACAGAAACTTCATGCAG |
| | CTGATTCACGACGACAGCCTCACCTTCAAAGAGGATATCCAGAAAGCCCAGGTGTCC |
| | GGCCAGGGCGATTCTCTGCATGAGCACATTGCCAACCTGGCCGGCTCTCCCGCCATT |
| | AAGAAAGGCATCCTGCAGACAGTGAAGGTGGTGGACGAGCTTGTGAAAGTGATGGGC |
| | GGACACAAGCCCGAGAACATCGTGATCGAAATGGCCAGAGAGAACCAGACCACACAG |
| | AAGGGACAGAAGAACAGCCGCGAGAGAATGAAGCGGATCGAAGAGGGCATCAAAGAG |
| | CTGGGCAGCCAGATCCTGAAAGAACACCCCGTGGAAAACACCCAGCTGCAGAACGAG |
| | AAGCTGTACCTGTACTACCTGCAGAATGGACGGGATATGTACGTGGACCAAGAGCTG |
| | GACATCAACAGACTGTCCGACTACGATGTGGACCATATCGTGCCCCAGTCTTTTCTG |
| | AAGGACGACTCCATCGACAACAAGGTCCTGACCAGATCCGACAAGAATCGGGGCAAG |
| | AGCGACAACGTGCCCTCCGAAGAGGTGGTCAAGAAGATGAAGAACTACTGGCGACAG |
| | CTGCTGAACGCCAAGCTGATTACCCAGCGGAAGTTCGACAATCTGACCAAGGCCGAA |
| | AGAGGCGGCCTGAGCGAACTGGATAAGGCCGGCTTCATCAAGAGACAGCTGGTGGAA |
| | ACCCGGCAGATCACAAAGCACGTGGCACAGATTCTGGACTCTCGGATGAACACTAAG |
| | TACGACGAGAACGACAAACTGATCCGCGAAGTGAAAGTCATCACCCTGAAGTCCAAG |
| | CTGGTGTCCGATTTCCGGAAGGATTTCCAGTTCTACAAAGTGCGCGAGATCAACAAC |
| | TACCATCACGCCCACGACGCCTACCTGAATGCCGTTGTTGGAACAGCCCTGATCAAA |
| | AAGTACCCTAAGCTGGAAAGCGAGTTCGTGTACGGCGACTACAAGGTGTACGACGTG |
| | CGGAAGATGATCGCCAAGAGCGAGCAAGAGATTGGCAAGGCAACCGCCAAGTACTTC |
| | TTCTACAGCAACATCATGAACTTTTTCAAGACAGAGATCACCCTCGCCAACGGCGAG |
| | ATCAGAAAGCGGCCTCTGATCGAGACAAACGGCGAAACCGGCGAGATTGTGTGGGAT |
| | AAGGGCAGAGACTTTGCCACAGTGCGGAAAGTGCTGAGCATGCCCCAAGTGAATATC |
| | GTGAAGAAAACCGAGGTGCAGACAGGCGGCTTCAGCAAAGAGTCTATCCTGCCTAAG |
| | GGGAACTCCGACAAGCTGATCGCCAGAAAGAAGGACTGGGACCCCAAGAAATACGGC |
| | GGCTTTAACTCTCCCACCGTGGCCTATTCTGTTCTGGTGGTGGCCAAAGTGGAAAAG |
| | GGCAAGTCCAAGAAACTCAAGAGCGTGAAAGAGCTGCTGGGGATCACCATCATGGAA |
| | AGAAGCAGCTTCGAGAAGAATCCGATCGATTTCCTCGAGGCCAAGGGTTACAAAGAA |
| | GTGAAAAAGGACCTGATCATCAAGCTCCCCAAGTACTCCCTGTTCGAGCTGGAAAAC |
| | GGCCGGAAGAGAATGCTGGCCTCTGCCGGTGTCCTGCAGAAGGGAAACGAACTGGCC |
| | CTGCCTAGCAAATATGTGAACTTCCTGTACCTGGCCAGCCACTATGAGAAGCTGAAG |
| | GGCAGCCCCGAGGACAATGAGCAAAAGCAGCTGTTTGTGGAACAGCACAAGCACTAC |
| | CTGGACGAGATCATCGAGCAGATCAGCGAGTTTAGCAAGAGAGTGATTCTGGCCGAC |
| | GCCAATCTGGACAAAGTGCTGTCCGCCTACAACAAGCACCGGGACAAGCCTATCAGA |
| | GAGCAGGCCGAGAATATCATCCACCTGTTTACCCTGACCAACCTGGGAGCCCCTGCG |
| | GCCTTCAAGTACTTTGACACCACCATCAACAGGAAGCAGTACAACACCACCAAAGAG |
| | GTGCTGGACGCCACTCTGATCCGCCAGTCTATCACCGGCCTGTACGAGACACGGATC |
| | GACCTGTCTCAACTCGGAGGCGACGAAGGCGCCGATAAGAGAACCGCCGATGGCTCT |
| | GAGTTCGAGAGCCCTAAGAAAAAGCGCAAAGTGTAG (SEQ ID NO: 942) |
| ABE9v2- | MSEVEFSHEYWMRHALTLAKRARDEREVPVGAVLVLNNRVIGEGWNRAIGLHDPTAH |
| NRCH | AEIMALRQGGLVMQNYRLYDATLYTTFEPCVMCAGAMIHSRIGRVVFGVRNAKTGAA |
| (ABE8.20 | GSLMDVLHHPGMNHRVEITEGILADECAALLCDFYRMPRRVFNAQKKAQSSINSGGS |
| w/ the | SGGSSGSETPGTSESATPESSGGSSGGSDKKYSIGLAIGTNSVGWAVITDEYKVPSK |
| alterations | KFKVLGNTDRHSIKKNLIGALLFDSGETAEATRLKRTARRRYTRRKNRICYLQEIFS |
| Y147D, | NEMAKVDDSFFHRLEESFLVEEDKKHERHPIFGNIVDEVAYHEKYPTIYHLRKKLVD |
| F149Y, | STDKADLRLIYLALAHMIKFRGHFLIEGDLNPDNSDVDKLFIQLVQTYNQLFEENPI |
| T166I, | NASGVDAKAILSARLSKSRRLENLIAQLPGEKKNGLFGNLIALSLGLTPNFKSNFDL |
| D167N and | AEDAKLQLSKDTYDDDLDNLLAQIGDQYADLFLAAKNLSDAILLSDILRVNTEITKA |
| S82T in the | PLSASMVKRYDEHHQDLTLLKALVRQQLPEKYKEIFFDQSKNGYAGYIDGGASQEEF |
| TadA* | YKFIKPILEKMDGTEELLVKLNREDLLRKQRTFDNGIIPHQIHLGELHAILRRQGDF |
| domain) | YPFLKDNREKIEKILTFRIPYYVGPLARGNSRFAWMTRKSEETITPWNFEEVVDKGA |
| | SAQSFIERMTNFDKNLPNEKVLPKHSLLYEYFTVYNELTKVKYVTEGMRKPAFLSGE |
| | QKKAIVDLLFKTNRKVTVKQLKEDYFKKIECFDSVEISGVEDRFNASLGTYHDLLKI |
| | IKDKDFLDNEENEDILEDIVLTLTLFEDREMIEERLKTYAHLFDDKVMKQLKRLRYT |
| | GWGRLSRKLINGIRDKQSGKTILDFLKSDGFANRNFMQLIHDDSLTFKEDIQKAQVS |
| | GQGDSLHEHIANLAGSPAIKKGILQTVKVVDELVKVMGGHKPENIVIEMARENQTTQ |
| | KGQKNSRERMKRIEEGIKELGSQILKEHPVENTQLQNEKLYLYYLQNGRDMYVDQEL |
| | DINRLSDYDVDHIVPQSFLKDDSIDNKVLTRSDKNRGKSDNVPSEEVVKKMKNYWRQ |
| | LLNAKLITQRKFDNLTKAERGGLSELDKAGFIKRQLVETRQITKHVAQILDSRMNTK |
| | YDENDKLIREVKVITLKSKLVSDFRKDFQFYKVREINNYHHAHDAYLNAVVGTALIK |
| | KYPKLESEFVYGDYKVYDVRKMIAKSEQEIGKATAKYFFYSNIMNFFKTEITLANGE |
| | IRKRPLIETNGETGEIVWDKGRDFATVRKVLSMPQVNIVKKTEVQTGGFSKESILPK |
| | GNSDKLIARKKDWDPKKYGGFNSPTVAYSVLVVAKVEKGKSKKLKSVKELLGITIME |
| | RSSFEKNPIDFLEAKGYKEVKKDLIIKLPKYSLFELENGRKRMLASAGVLQKGNELA |
| | LPSKYVNFLYLASHYEKLKGSPEDNEQKQLFVEQHKHYLDEIIEQISEFSKRVILAD |
| | ANLDKVLSAYNKHRDKPIREQAENIIHLFTLTNLGAPAAFKYFDTTINRKQYNTTKE |
| | VLDATLIRQSITGLYETRIDLSQLGGDEGADKRTADGSEFESPKKKRKV (SEQ ID |
| | NO: 935) |
| ABE9v2- | ATGTCCGAAGTCGAGTTTTCCCATGAGTACTGGATGAGACACGCATTGACTCTCGCA |
| NRCH | AAGAGGGCTCGAGATGAACGCGAGGTGCCCGTGGGGGCAGTACTCGTGCTCAACAAT |
| | CGCGTAATCGGCGAAGGTTGGAATAGGGCAATCGGACTCCACGACCCCACTGCACAT |
| | GCGGAAATCATGGCCCTTCGACAGGGAGGGCTTGTGATGCAGAATTATCGACTTTAT |
| | GATGCGACGCTGTACACCACGTTTGAACCTTGCGTAATGTGCGCGGGAGCTATGATT |
| | CACTCCCGCATTGGACGAGTTGTATTCGGTGTTCGCAACGCCAAGACGGGTGCCGCA |
| | GGTTCACTGATGGACGTGCTGCATCATCCAGGCATGAACCACCGGGTAGAAATCACA |
| | GAAGGCATATTGGCGGACGAATGTGCGGCGCTGTTGTGTGACTTTTATCGCATGCCC |
| | AGGCGTGTCTTTAACGCCCAGAAAAAAGCACAATCCTCTATAAACTCCGGCGGAAGC |
| | AGCGGAGGATCTTCTGGAAGCGAAACCCCAGGCACCAGCGAGTCTGCCACACCAGAA |
| | TCATCTGGCGGTAGCTCCGGCGGCAGCGACAAGAAGTATTCTATCGGACTGGCCATC |
| | GGCACCAACTCTGTTGGATGGGCCGTGATCACCGACGAGTACAAGGTGCCCAGCAAG |
| | AAATTCAAGGTGCTGGGCAACACCGACAGGCACAGCATCAAGAAGAACCTGATCGGC |
| | GCACTGCTGTTCGACTCTGGCGAAACAGCCGAGGCCACCAGACTGAAGAGAACAGCC |
| | CGCAGACGGTACACCAGAAGAAAGAACCGGATCTGCTACCTCCAAGAGATCTTCAGC |
| | AACGAGATGGCCAAGGTGGACGACAGCTTCTTCCACAGACTGGAAGAGTCCTTCCTG |
| | GTGGAAGAGGACAAGAAGCACGAGAGACACCCCATCTTCGGCAACATCGTGGACGAG |
| | GTGGCCTACCACGAGAAGTACCCCACCATCTACCACCTGAGAAAGAAACTGGTGGAC |
| | AGCACCGACAAGGCCGACCTGAGACTGATCTATCTGGCCCTGGCTCACATGATCAAG |
| | TTCCGGGGCCACTTCCTGATCGAGGGCGACCTGAATCCTGACAACAGCGACGTGGAC |
| | AAGCTGTTCATCCAGCTGGTGCAGACCTACAACCAGCTGTTCGAGGAAAACCCCATC |
| | AACGCCAGCGGAGTGGATGCCAAGGCCATCCTGTCTGCCAGACTGAGCAAGAGCAGA |
| | CGGCTGGAAAATCTGATCGCCCAGCTGCCTGGCGAGAAGAAGAATGGCCTGTTCGGC |
| | AACCTGATTGCCCTGAGCCTGGGCCTGACACCTAACTTCAAGAGCAACTTCGACCTG |
| | GCCGAGGACGCCAAACTGCAGCTGAGCAAGGACACCTACGACGACGACCTGGACAAT |
| | CTGCTGGCCCAGATCGGCGATCAGTACGCCGACTTGTTTCTGGCCGCCAAGAATCTG |
| | AGCGACGCCATCCTGCTGTCCGACATCCTGAGAGTGAACACCGAGATCACCAAGGCA |
| | CCTCTGAGCGCCTCTATGGTCAAGAGATACGACGAGCACCACCAGGATCTGACCCTG |
| | CTGAAGGCCCTCGTTAGACAGCAGCTGCCAGAGAAGTACAAAGAGATTTTCTTCGAC |
| | CAGAGCAAGAACGGCTACGCCGGCTACATTGATGGCGGAGCCAGCCAAGAGGAATTC |
| | TACAAGTTCATCAAGCCCATCCTCGAGAAGATGGACGGCACCGAGGAACTGCTGGTC |
| | AAGCTGAACAGAGAGGACCTGCTGAGAAAGCAGAGAACCTTCGACAACGGCATCATC |
| | CCTCACCAGATCCACCTGGGAGAACTGCACGCCATTCTGCGGAGACAAGGGGACTTT |
| | TACCCATTCCTGAAGGACAACCGGGAAAAGATCGAGAAAATCCTGACCTTCAGGATC |
| | CCCTACTACGTGGGACCACTGGCCAGAGGCAATAGCAGATTCGCCTGGATGACCAGA |
| | AAGAGCGAGGAAACCATCACTCCCTGGAACTTCGAGGAAGTGGTGGACAAGGGCGCC |
| | AGCGCTCAGTCCTTCATCGAGCGGATGACCAACTTCGATAAGAACCTGCCTAACGAG |
| | AAGGTGCTGCCCAAGCACAGCCTGCTGTACGAGTACTTCACCGTGTACAACGAGCTG |
| | ACCAAAGTGAAATACGTGACCGAGGGAATGAGAAAGCCCGCCTTTCTGAGCGGCGAG |
| | CAGAAAAAGGCCATCGTGGATCTGCTGTTCAAGACCAACCGGAAAGTGACCGTGAAG |
| | CAGCTGAAAGAGGACTACTTCAAGAAAATCGAGTGCTTCGACAGCGTCGAGATCTCC |
| | GGCGTGGAAGATCGGTTCAATGCCAGCCTGGGCACATACCACGATCTGCTGAAAATT |
| | ATCAAGGACAAGGACTTCCTGGACAACGAAGAGAACGAGGACATCCTTGAGGACATC |
| | GTGCTGACACTGACCCTGTTTGAGGACAGAGAGATGATCGAGGAACGGCTGAAAACA |
| | TACGCCCACCTGTTCGACGACAAAGTGATGAAGCAACTGAAGCGGCTGAGATACACC |
| | GGCTGGGGCAGACTGTCTCGGAAGCTGATCAACGGCATCCGGGATAAGCAGTCCGGC |
| | AAGACCATCCTGGACTTTCTGAAGTCCGACGGCTTCGCCAACAGAAACTTCATGCAG |
| | CTGATTCACGACGACAGCCTCACCTTCAAAGAGGATATCCAGAAAGCCCAGGTGTCC |
| | GGCCAGGGCGATTCTCTGCATGAGCACATTGCCAACCTGGCCGGCTCTCCCGCCATT |
| | AAGAAAGGCATCCTGCAGACAGTGAAGGTGGTGGACGAGCTTGTGAAAGTGATGGGC |
| | GGACACAAGCCCGAGAACATCGTGATCGAAATGGCCAGAGAGAACCAGACCACACAG |
| | AAGGGACAGAAGAACAGCCGCGAGAGAATGAAGCGGATCGAAGAGGGCATCAAAGAG |
| | CTGGGCAGCCAGATCCTGAAAGAACACCCCGTGGAAAACACCCAGCTGCAGAACGAG |
| | AAGCTGTACCTGTACTACCTGCAGAATGGACGGGATATGTACGTGGACCAAGAGCTG |
| | GACATCAACAGACTGTCCGACTACGATGTGGACCATATCGTGCCCCAGTCTTTTCTG |
| | AAGGACGACTCCATCGACAACAAGGTCCTGACCAGATCCGACAAGAATCGGGGCAAG |
| | AGCGACAACGTGCCCTCCGAAGAGGTGGTCAAGAAGATGAAGAACTACTGGCGACAG |
| | CTGCTGAACGCCAAGCTGATTACCCAGCGGAAGTTCGACAATCTGACCAAGGCCGAA |
| | AGAGGCGGCCTGAGCGAACTGGATAAGGCCGGCTTCATCAAGAGACAGCTGGTGGAA |
| | ACCCGGCAGATCACAAAGCACGTGGCACAGATTCTGGACTCTCGGATGAACACTAAG |
| | TACGACGAGAACGACAAACTGATCCGCGAAGTGAAAGTCATCACCCTGAAGTCCAAG |
| | CTGGTGTCCGATTTCCGGAAGGATTTCCAGTTCTACAAAGTGCGCGAGATCAACAAC |
| | TACCATCACGCCCACGACGCCTACCTGAATGCCGTTGTTGGAACAGCCCTGATCAAA |
| | AAGTACCCTAAGCTGGAAAGCGAGTTCGTGTACGGCGACTACAAGGTGTACGACGTG |
| | CGGAAGATGATCGCCAAGAGCGAGCAAGAGATTGGCAAGGCAACCGCCAAGTACTTC |
| | TTCTACAGCAACATCATGAACTTTTTCAAGACAGAGATCACCCTCGCCAACGGCGAG |
| | ATCAGAAAGCGGCCTCTGATCGAGACAAACGGCGAAACCGGCGAGATTGTGTGGGAT |
| | AAGGGCAGAGACTTTGCCACAGTGCGGAAAGTGCTGAGCATGCCCCAAGTGAATATC |
| | GTGAAGAAAACCGAGGTGCAGACAGGCGGCTTCAGCAAAGAGTCTATCCTGCCTAAG |
| | GGGAACTCCGACAAGCTGATCGCCAGAAAGAAGGACTGGGACCCCAAGAAATACGGC |
| | GGCTTTAACTCTCCCACCGTGGCCTATTCTGTTCTGGTGGTGGCCAAAGTGGAAAAG |
| | GGCAAGTCCAAGAAACTCAAGAGCGTGAAAGAGCTGCTGGGGATCACCATCATGGAA |
| | AGAAGCAGCTTCGAGAAGAATCCGATCGATTTCCTCGAGGCCAAGGGTTACAAAGAA |
| | GTGAAAAAGGACCTGATCATCAAGCTCCCCAAGTACTCCCTGTTCGAGCTGGAAAAC |
| | GGCCGGAAGAGAATGCTGGCCTCTGCCGGTGTCCTGCAGAAGGGAAACGAACTGGCC |
| | CTGCCTAGCAAATATGTGAACTTCCTGTACCTGGCCAGCCACTATGAGAAGCTGAAG |
| | GGCAGCCCCGAGGACAATGAGCAAAAGCAGCTGTTTGTGGAACAGCACAAGCACTAC |
| | CTGGACGAGATCATCGAGCAGATCAGCGAGTTTAGCAAGAGAGTGATTCTGGCCGAC |
| | GCCAATCTGGACAAAGTGCTGTCCGCCTACAACAAGCACCGGGACAAGCCTATCAGA |
| | GAGCAGGCCGAGAATATCATCCACCTGTTTACCCTGACCAACCTGGGAGCCCCTGCG |
| | GCCTTCAAGTACTTTGACACCACCATCAACAGGAAGCAGTACAACACCACCAAAGAG |
| | GTGCTGGACGCCACTCTGATCCGCCAGTCTATCACCGGCCTGTACGAGACACGGATC |
| | GACCTGTCTCAACTCGGAGGCGACGAAGGCGCCGATAAGAGAACCGCCGATGGCTCT |
| | GAGTTCGAGAGCCCTAAGAAAAAGCGCAAAGTGTAG (SEQ ID NO: 943) |

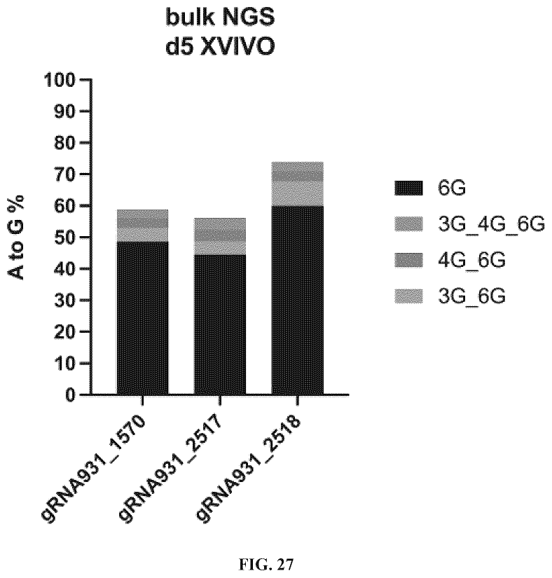

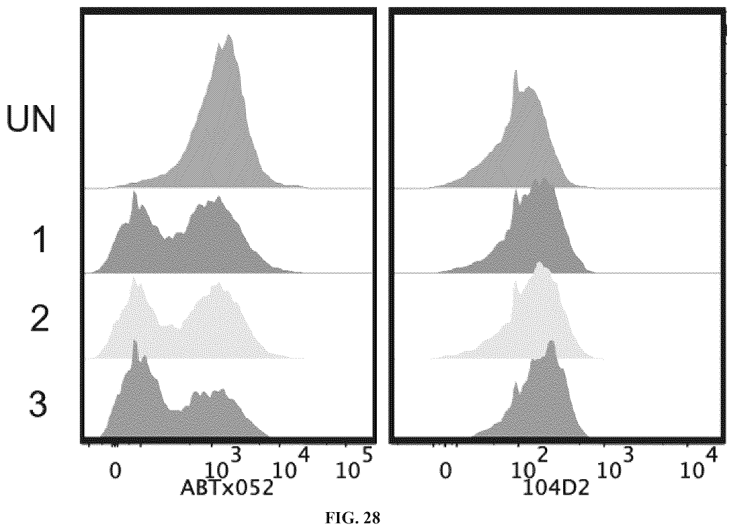

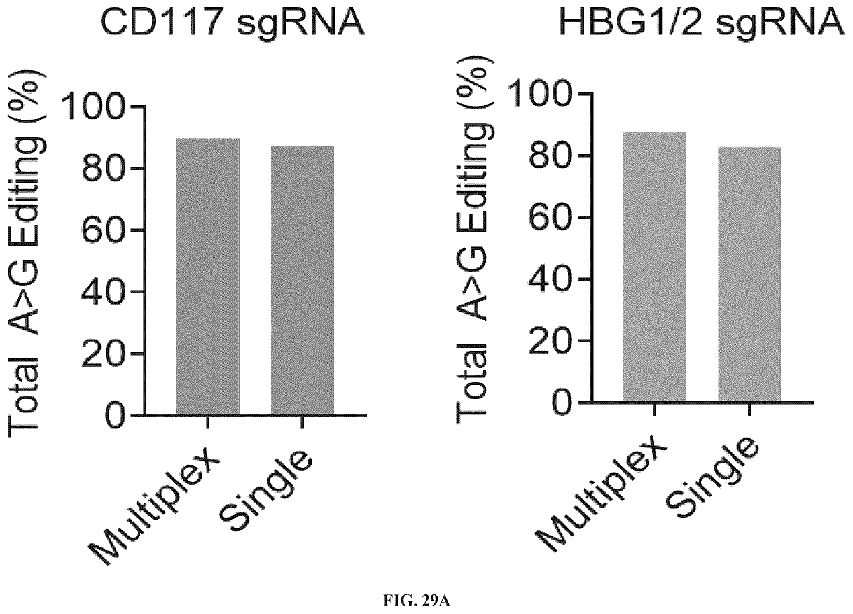

Experiments were undertaken to evaluate editing efficiencies in CD34+ hematopoietic stem cells (HSCs) using the base editors ABE8.20, ABE9v1, and ABE9v2 (see Table 17) in combination with the guide gRNA931 (CC200). The cells were transfected with the guide and mRNA encoding the base editors using electroporation. The cells were grown in XVIVO serum free stem cell medium. All of the combinations evaluated resulted in editing efficiencies of greater than 50% (see FIG. 27). The resulting edited cells showed improved escape from ABTx052 binding 2 days post-electroporation (FIG. 28).

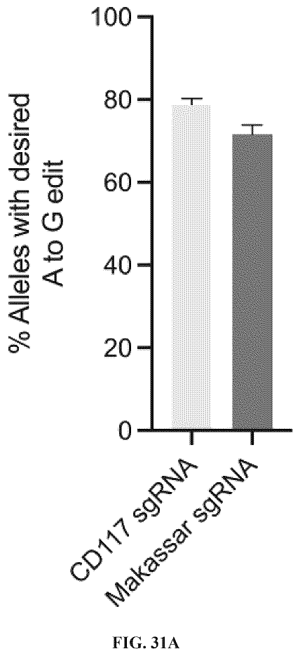

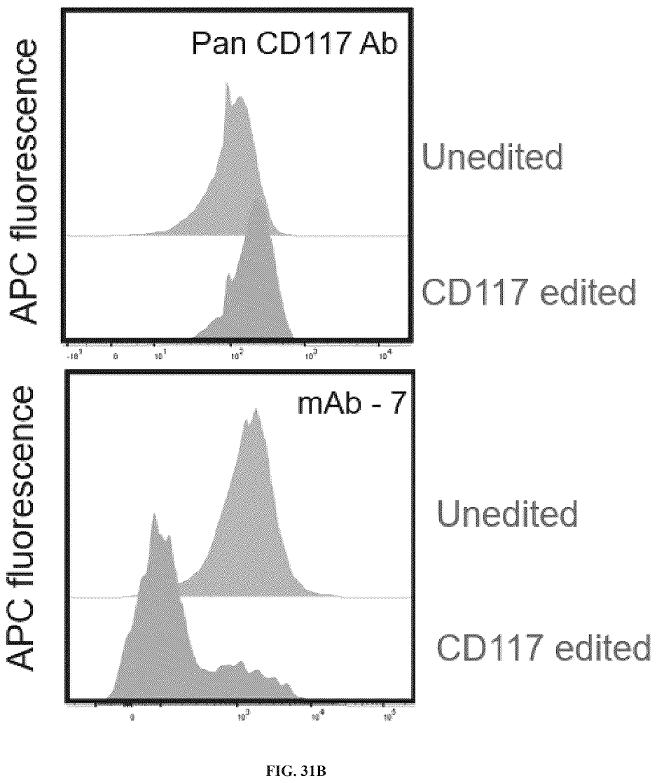

The base editors used to edit the CD117 polynucleotide were suitable for use in introducing a Makassar edit to a beta globin gene. CD34+ hematopoietic stem cells were edited in multiplex by contacting them with an mRNA polynucleotide encoding an adenosine deaminase (see ABE8.20+, ABE9v1, and ABE9v2 of Table 17) and the guide RNAs sgRNA_027 and gRNA931. High editing levels of base editing were achieved at the beta globin gene (HBB) locus for sickle to Makassar gene correction (FIG. 31A). The multiplex edited cells showed reduced binding to mAb-7 (i.e., ABTx052) (FIG. 31B). Therefore, high levels of base editing at the HBB gene locus for sickle to Makassar gene correction and CD117 was achieved using a single ABE.

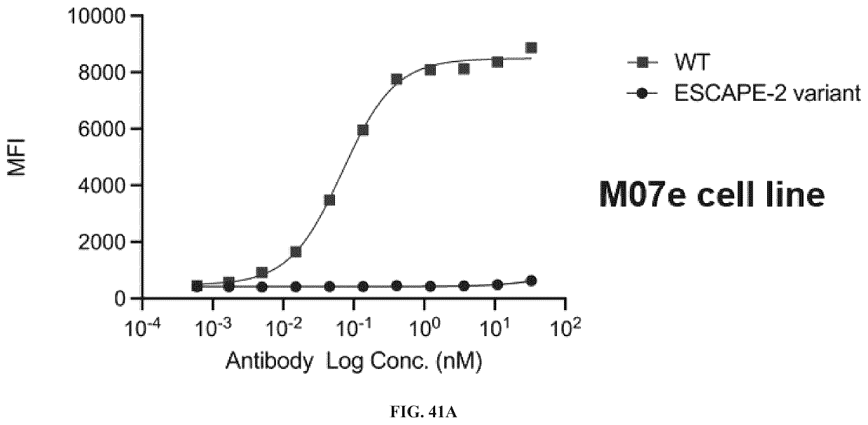

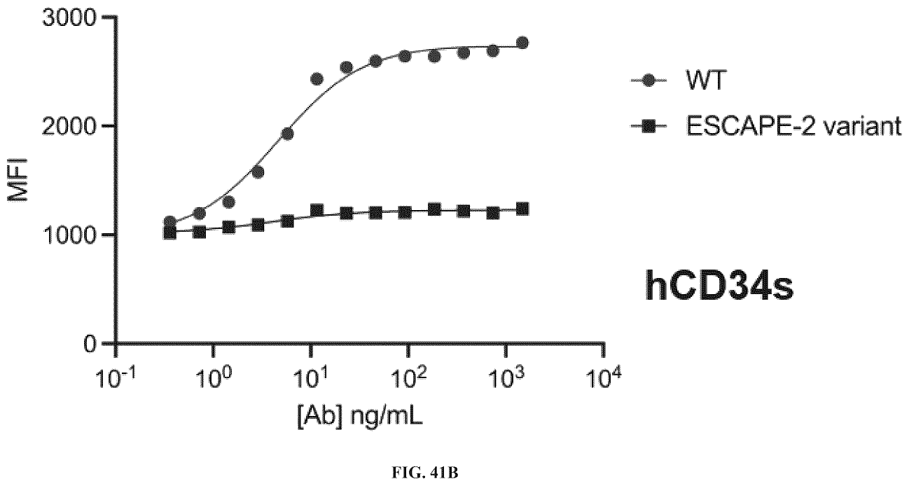

An experiment was undertaken to evaluate binding of ABTx052 to M07e and hCD34+ cells surface-expressing either WT CD117 or a CD117 variant prepared by base editing the cells using an adenosine deaminase (ABE9v2) and gRNA931. It was determined that ABTx052 bound with high affinity to cells expressing WT CD117 while the base-edited cells expressing the CD117 variant (ESCAPE-2 variant) escaped recognition by ABTx052 at all concentrations of the antibody that were evaluated (FIGS. 41A and 41B).

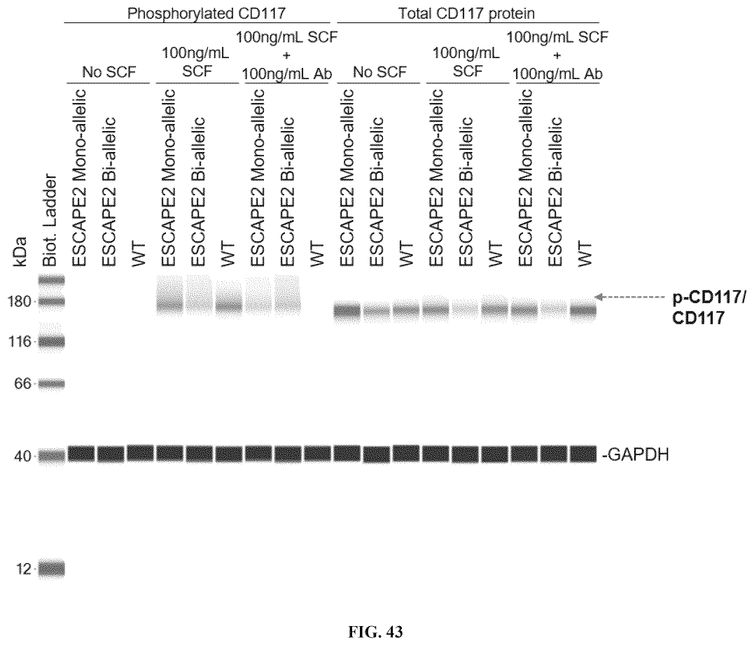

A further experiment was undertaken in vitro to determine whether the altered CD117 polypeptide expressed in CD34+ hematopoietic stem cells base edited using gRNA931 was fully functional. The cells were multiplex base edited using an adenosine deaminase and the guide polynucleotides sgRNA_027 and gRNA931. The cells were grown in vitro, and it was assessed whether the altered CD117 polypeptide was capable of binding SCF and whether the SCF binding induced phosphorylation. It was determined that SCF did bind to and induce phosphorylation of the altered CD117 polypeptide expressed by the CD34+ cells, even in the presence of the ABTx052 antibody (FIG. 43). The cells evaluated were either bi-allelic or mono-allelic for the altered CD117 polypeptide, and both cell types were signaling competent. Therefore, the epitope alteration contained in the altered CD117 polypeptide did not interfere with normal receptor-ligand binding.

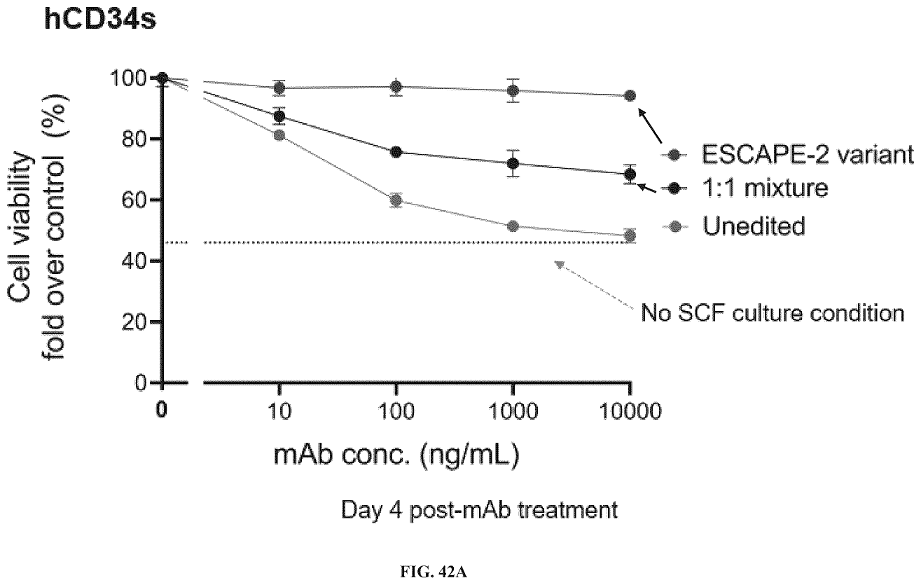

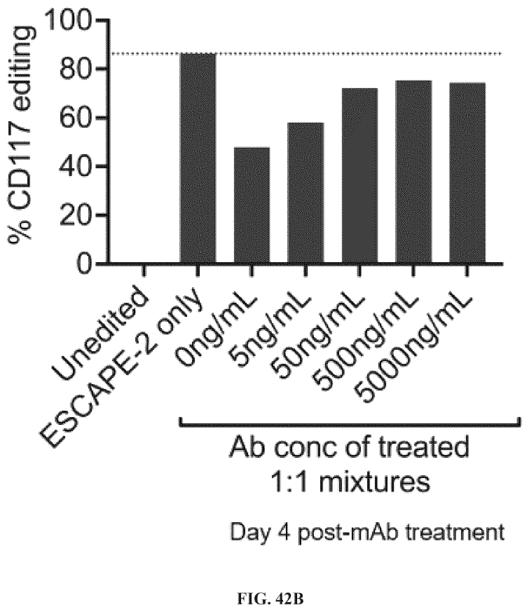

The multiplex base edited primary hCD34+HSPCs prepared as described above using an adenosine deaminase and sgRNA_027 and gRNA931 were evaluated to determine if the cells were able to escape depletion by ABTx052. It was determined that the cells did, in fact, escape depletion (FIGS. 42A and 42B). In an in vitro competition assay, ABTx052 mediated enrichment of cells expressing the altered CD117 polypeptide produced through base editing when grown in co-culture with unedited cells at an initial edited cells to unedited cells ratio of 1:1. Unedited cells reached levels of SCF withdrawal conditions in a dose-dependent fashion, whereas the multiplex base edited CD34+ cells maintained viability (FIG. 42A). Enrichment of the CD117 variant was confirmed using next-generation sequencing (NGS) (FIG. 42B).

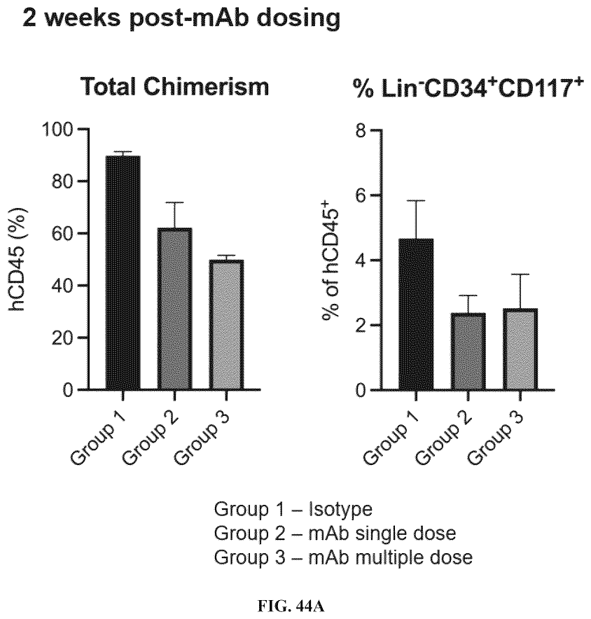

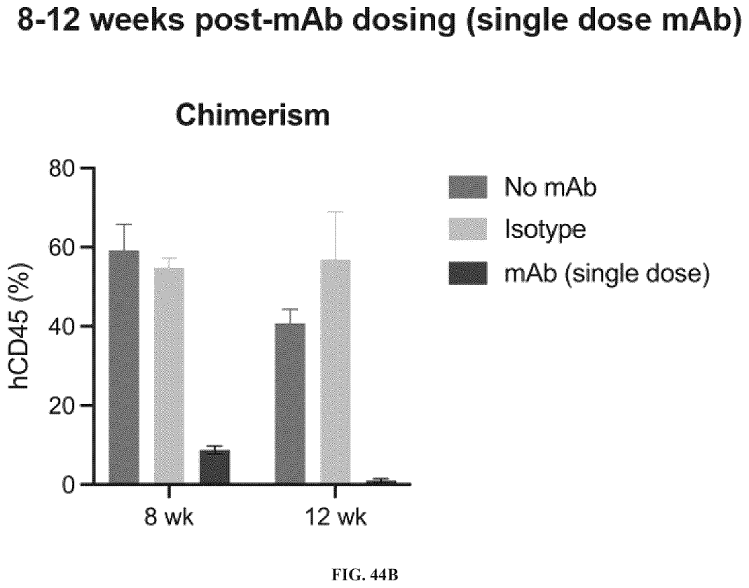

Next, in vivo depletion of wild type hematopoietic stem cells (HSCs) in the bone marrow of NSG mice mediated by ABTx052 (FIG. 44A) or ABTx135 (FIG. 44B) administration (20 mg antibody/kg mouse body weight) was evaluated. At 4 weeks post-humanization (i.e., post-administration of the HSCs), the NSG mice were administered the monoclonal antibody (mAb) ABTx052 or ABTx135. Chimerism was measured at two weeks following administration of ABTx052 (FIG. 44A) and at 8 and 12 weeks following administration of ABTx135 (FIG. 44B). Significant reductions in chimerism was observed in mice treated with ABTx052 at 2 weeks post-administration of the antibody. At longer time points, even with a single dose of ABTx135, even further reductions in chimerism were observed, suggesting that ABTx135 could abrogate long-term engraftment of wild-type HSPCs in vivo.

The above-presented results demonstrate the development of a pair containing a base-edited antigen and a corresponding antibody that enables edited cells to “ESCAPE” binding from a monoclonal antibody (mAb) that can deplete unedited HSCs both in vitro and in vivo. Combined with a therapeutic Makassar edit that eliminate the causative sickle cell disease protein, the ESCAPE strategy presents a promising new paradigm for autologous stem cell therapies in treatment of sickle cell anemia.

The Hb G-Makassar-compatible ESCAPE strategy presented above demonstrated not only highly efficient editing of CD117 polynucleotides in HSCs, but also the ability to evade binding by a monoclonal antibody and subsequent depletion mediated thereby in vitro. The above experiments demonstrate that primary human HSPCs harboring the engineered CD117 epitope could effectively evade depletion mediated by the blocking of the CD117 ligand (SCF) binding by a highly specific and potent mAb in vitro. Biological assessment of receptor function suggested that the engineered CD117 epitope was compatible with normal function. In vivo studies that were conducted also suggested the mAb was effective in depleting non-edited HSPCs.

With the above-described non-genotoxic conditioning approach, the direct editing of the causative sickle cell mutation to the naturally occurring and asymptomatic Hb G-Makassar is a promising new potential treatment paradigm for autologous hematopoietic stem cell transplant (HSCT) for patients with sickle cell anemia.

### Example 3: Cryo-Electron Microscopy (EM) Structures

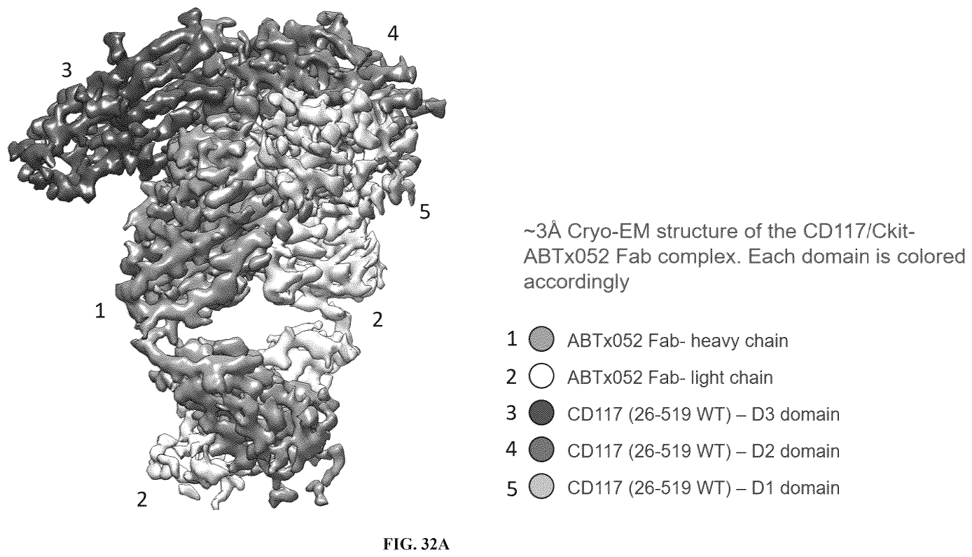

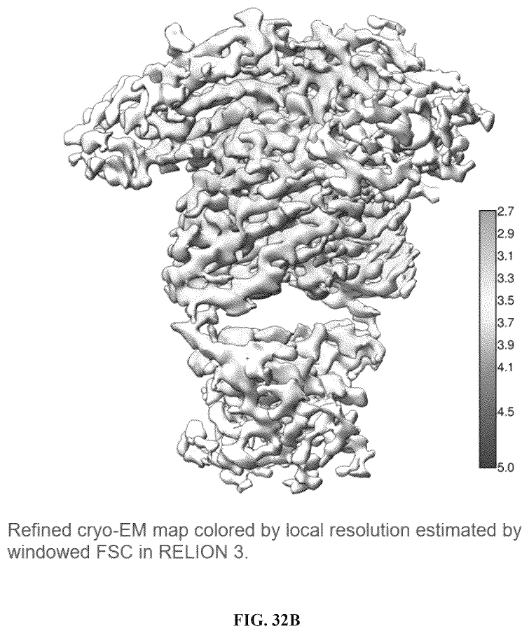

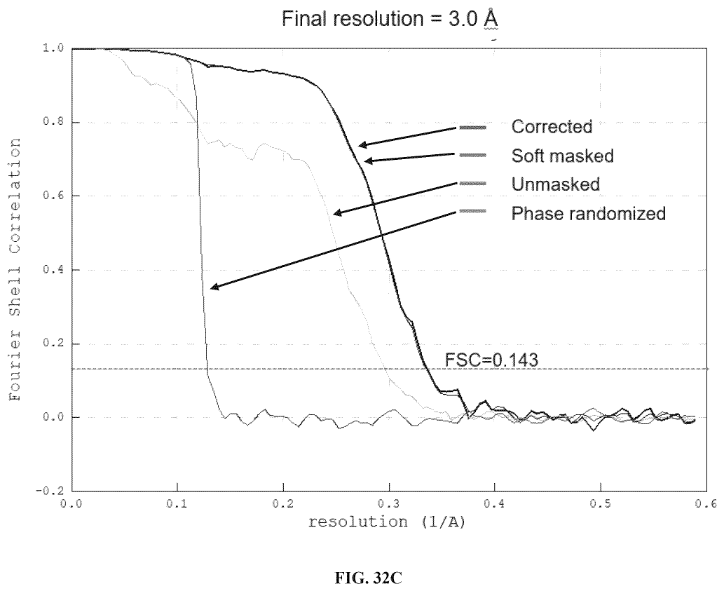

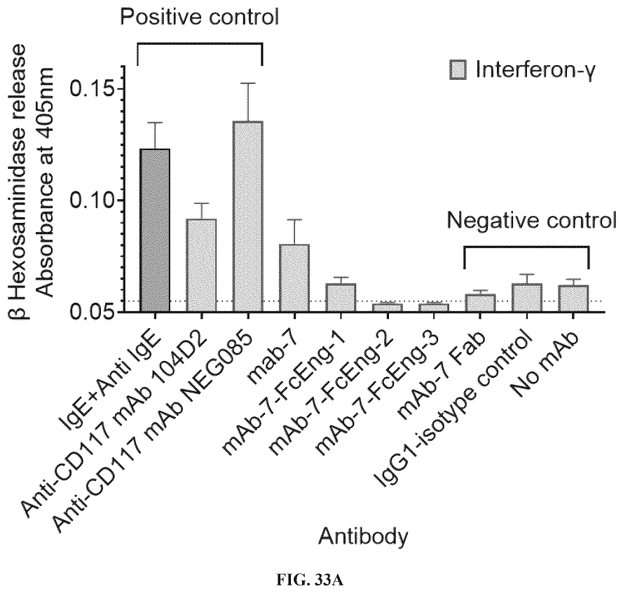

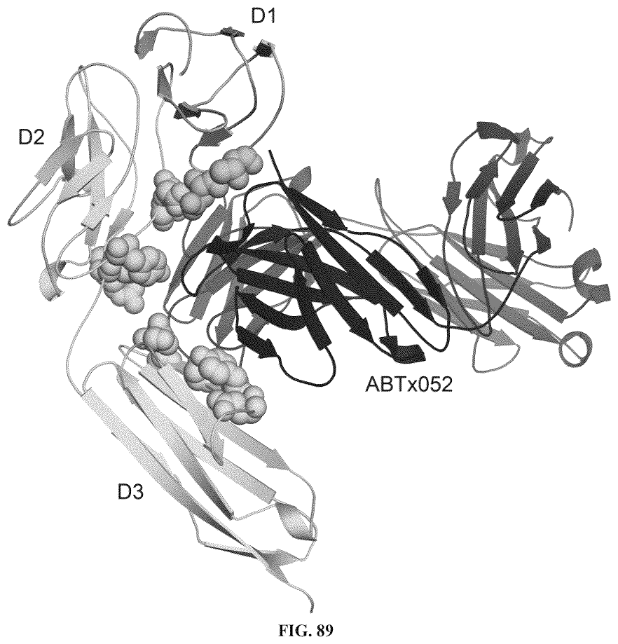

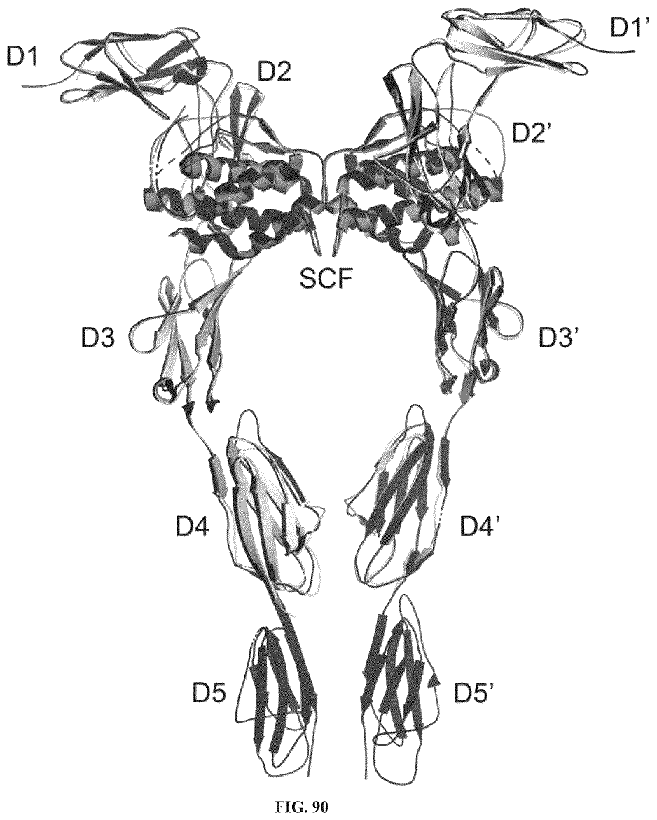

To determine the structure of ABTx052 bound to CD117, cryo-electron microscopy (cryo-EM) was utilized to determine a ˜3.0 Å structure of CD117/c-KIT in complex with ABTx052-Fab in solution state (FIGS. 32A-32C). Also, FIGS. 89 and 90 provide ribbon diagrams showing structures of CD117 bound by ABTx052 and of stem cell factor (SCF) bound to CD117, respectively, as determined using cryo-EM. High structural similarity between complexes of wild type CD117 or the CC128 variant of CD117 with SCF showed that the CC128-Y259C—N260D substitutions in the CC128 variant did not affect the protein structure and, consequently, its function in binding SCF.

### Example 4: Modifications to ABTx052 to Reduce Mast Cell Degranulation

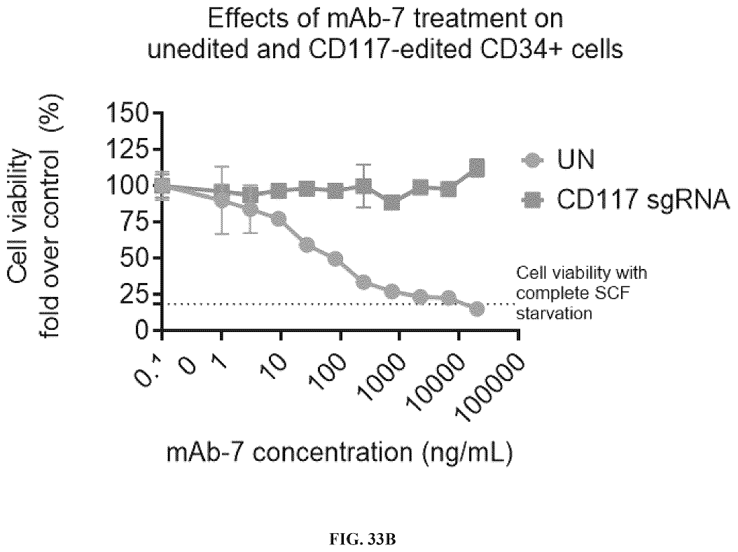

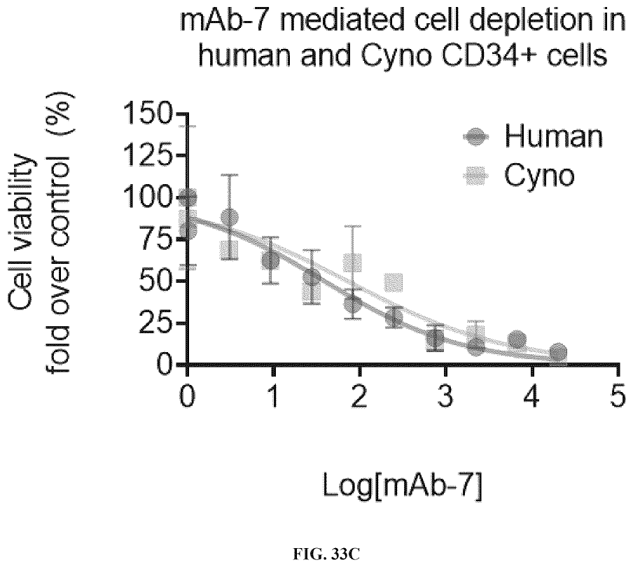

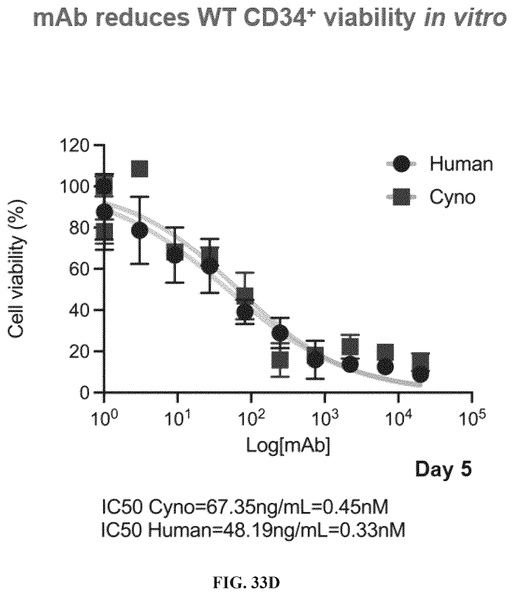

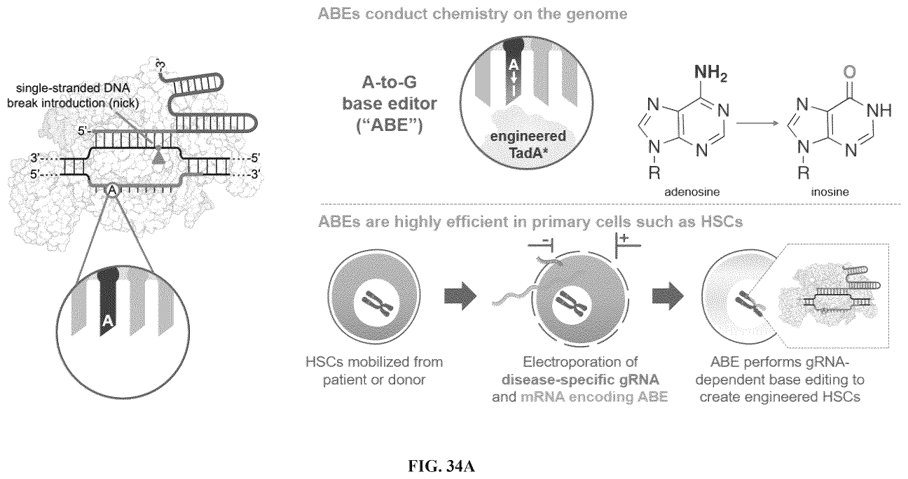

Experiments were undertaken to determine what modifications to the Fc domain of ABTx052 would reduce the degranulation of mast cells contacted with the antibody. ABTx052 variants containing altered Fc domains were prepared. In particular mAb-7-FcEng-1, mAB-7-FcEng-3, and mAb-7-FcEng-3, were prepared, which correspond to ABTx052 modified to contain the Fc modifications L234A, L235A (LALA); L234A, L235A, P329G (LALAPG); L234S, L235T, G236R (LSLTGR); and L234A, L235A, D265A (LALADA), respectively, referenced to the following amino acid sequence: SGAAVKKPGESLKISCKGSGYRFTSYWIGWVRQMPGKGLEWMGI IYPGDSDTRYSPSFQGQVTI SAGKSISTAYLOWSSLKASDTAMYYCARHGRGYNGYEGAFDIWGQGTMVTVSSASTKGPSVFPL APSSKSTSGGTAALGCLVKDYFPEPVTVSWNSGALTSGVHTF PAVLQSSGLYSLSSVVTVPSSS LGTQTYI CNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFYPSDIAVEWESN GQPENNYKTTPPVLDSDGSFFLYSKLTVDKSRWQQGNVFSCSVMHEALHNHYTQKSLSLSPG (positions 7-1101 of SEQ ID NO: 419). Degranulation of mast cells in the presence of the ABTx052 variants and control antibodies was measured using a mast cell degranulation assay. In brief, CD34+ cells were differentiated into mast cells using established protocols, the cells were then sensitized with human IgE or treated with interferon gamma, which enhances the expression of Fcgamma receptor on the cell surface. The cells were then contacted with the antibodies and cell degranulation was measured by detecting betahexosaminidase release in the supernatant. Mast cells sensitized with IgE showed degranulation when incubated with anti-human IgE, and this served as a positive control for the assay (FIG. 33A). The positive control antibodies 104D2 and NEG085 were known previously to cause mast cell degranulation upon incubation with mast cells. Unmodified ABTx052 antibody led to some mast cell degranulation, in vitro. It was determined that the ABTx052 variants resulted in reduced levels of mast cell degranulation relative to the unmodified ABTx052 antibody (FIG. 33A), as measured by reduced levels of beta hexosaminidase release in vitro. It was also determined that ABTx052 selectively depleted unedited CD34+ cells in vitro, while CD117-edited cells retained viability (FIG. 33B). It was found that the ABTx135 (ABTx052 with a LALADA alteration in the Fc domain) effected similar levels of cell depletion in both human and Cynomolgus CD34+ cells (FIGS. 33C and 33D). Cells contacted with ABTx052 or the ABTx052 Fc variants mimicked complete SCF withdrawal.

### Example 5: ABTx052 Variants that Bound Wild-Type CD117 and that Showed Low-to-No Binding to CD117 Variants

Experiments were undertaken to identify ABTx052 variants that were capable of binding wild-type CD117 while showing reduced binding to the CD117 variants CC128 and CC200. The CC128 and CC200 variants of CD117 were expressed from polynucleotides prepared by base editing a polynucleotide encoding wild-type CD117 using a base editor system containing the guide polynucleotide CC128 or CC200, respectively, and an adenosine deaminase base editor (ABE). The ABTx052 variants each contained a single amino acid alteration in either the heavy chain variable region (VH) or the light chain variable region (VL) of ABTx052, where the alteration was at one of the positions highlighted in bold in the below VH and VL sequences:

ABTx052 Heavy chain variable region (VH)
(SEQ ID NO: 959)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYNGYEGAFDIWGQGTMVTVSS
ABTx052 Light chain variable region (VL)
(SEQ ID NO: 960)
AIQLTQSPSSLSASVGDRVTITCRASQGISSALAWYQQKPGKAPKLLIY
DASSLESGVPSRFSGSGSGTDFTLTISSLQPEDFATYYCQQFNSYPLTF
GGGTKVEIKRTV.

In particular, ABTx052 variants containing the following alterations in the heavy chain variable region (VH) or in the light chain variable region (VL) were prepared: VH_R59K, VH_N104D, VH_G105A, VH_E107D (ABTx181), VL_F91T, VL_F91V, VL_N92R, VL_N92Y, VL_S93G, VL_Y94H, and VL_Y94S. Not intending to be bound by theory, amnio acid position N104 is a deamination site. Three of the ABTx052 variants contained a VH CDR3 with one of the following amino acid sequences: ARHGRGYDGYEGAFDI (SEQ ID NO: 948), ARHGRGYNAYEGAFDI (SEQ ID NO: 949), ARHGRGYNGYDGAFDI (SEQ ID NO: 950). One of the ABTx052 variants contained a VH CDR2 with the following amino acid sequence: IYPGDSDTK (SEQ ID NO: 972). Seven of the ABTx052 variants contained a VL CDR3 with one of the following amino acid sequences: QQTNSYPLT (SEQ ID NO: 951), QQVNSYPLT (SEQ ID NO: 952), QQFRSYPLT (SEQ ID NO: 953), QQFYSYPLT (SEQ ID NO: 954), QQFNGYPLT (SEQ ID NO: 957), QQFNSHPLT (SEQ ID NO: 955), QQFNSSPLT (SEQ ID NO: 956). Amino acid sequences for variable light chains and variable heavy chains corresponding to the ABTx052 variants are provide below:

VH_R59K:
(SEQ ID NO: 961)
QVQLVQSGAAVKKPGESLKISCKGSGYRFTSYWIGWVRQMPGKGLEWMG
IIYPGDSDTKYSPSFQGQVTISAGKSISTAYLQWSSLKASDTAMYYCAR
HGRGYNGYEGAFDIWGQGTMVTVSS.
VH_N104D:
(SEQ ID NO: 962)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYDGYEGAFDIWGQGTMVTVSS.
VH_G105A:
(SEQ ID NO: 963)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYNAYEGAFDIWGQGTMVTVSS.
VH_E107D (ABTx181):
(SEQ ID NO: 964)
QVQLVQSGAAVKKPGESLKISCKGSGYRFTSYWIGWVRQMPGKGLEWMG
IIYPGDSDTRYSPSFQGQVTISAGKSISTAYLQWSSLKASDTAMYYCAR
HGRGYNGYDGAFDIWGQGTMVTVSS.
VL_F91T:
(SEQ ID NO: 965)
AIQLTQSPSSLSASVGDRVTITCRASQGISSALAWYQQKPGKAPKLLIY
DASSLESGVPSRFSGSGSGTDFTLTISSLQPEDFATYYCQQTNSYPLTF
GGGTKVEIK.
VL_F91V:
(SEQ ID NO: 966)
AIQLTQSPSSLSASVGDRVTITCRASQGISSALAWYQQKPGKAPKLLIY
DASSLESGVPSRFSGSGSGTDFTLTISSLQPEDFATYYCQQVNSYPLTF
GGGTKVEIK.
VL_N92R:
(SEQ ID NO: 967)
AIQLTQSPSSLSASVGDRVTITCRASQGISSALAWYQQKPGKAPKLLIY
DASSLESGVPSRFSGSGSGTDFTLTISSLQPEDFATYYCQQFRSYPLTF
GGGTKVEIK.
VL_N92Y:
(SEQ ID NO: 968)
AIQLTQSPSSLSASVGDRVTITCRASQGISSALAWYQQKPGKAPKLLIY
DASSLESGVPSRFSGSGSGTDFTLTISSLQPEDFATYYCQQFYSYPLTF
GGGTKVEIK.
VL_S93G:
(SEQ ID NO: 969)
AIQLTQSPSSLSASVGDRVTITCRASQGISSALAWYQQKPGKAPKLLIY
DASSLESGVPSRFSGSGSGTDFTLTISSLQPEDFATYYCQQFNGYPLTF
GGGTKVEIK.
VL_Y94H:
(SEQ ID NO: 970)
AIQLTQSPSSLSASVGDRVTITCRASQGISSALAWYQQKPGKAPKLLIY
DASSLESGVPSRFSGSGSGTDFTLTISSLQPEDFATYYCQQFNSHPLTF
GGGTKVEIK.
VL_Y94S:
(SEQ ID NO: 971)
AIQLTQSPSSLSASVGDRVTITCRASQGISSALAWYQQKPGKAPKLLIY
DASSLESGVPSRFSGSGSGTDFTLTISSLQPEDFATYYCQQFNSSPLTF
GGGTKVEIK.

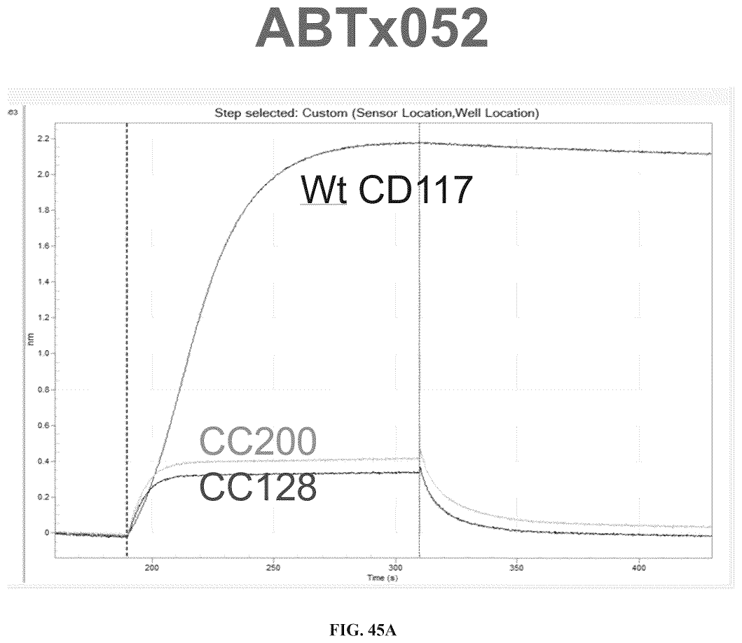

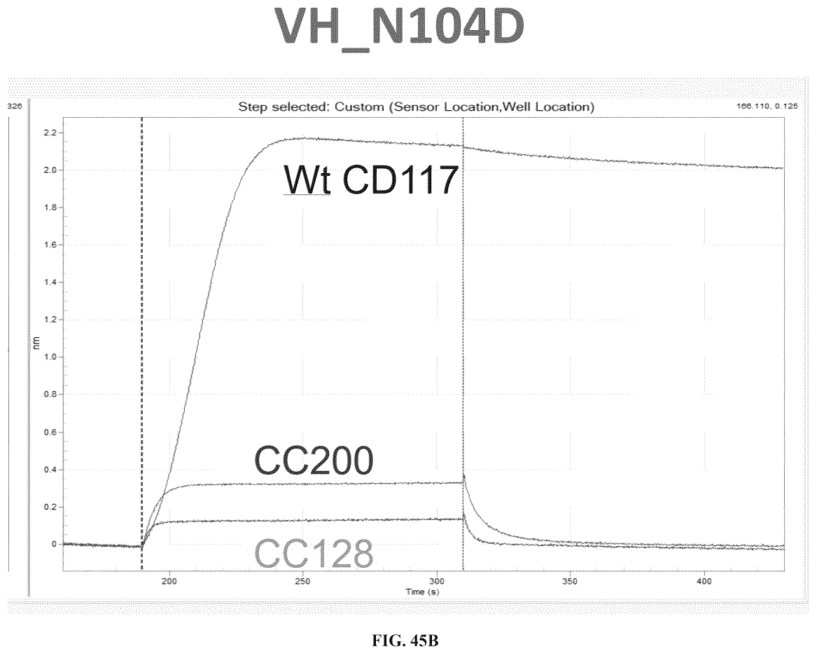

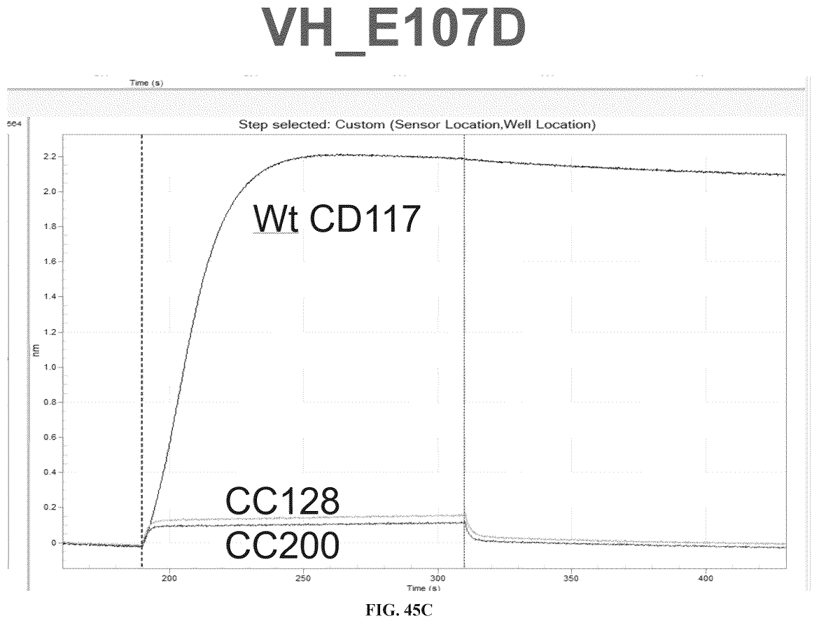

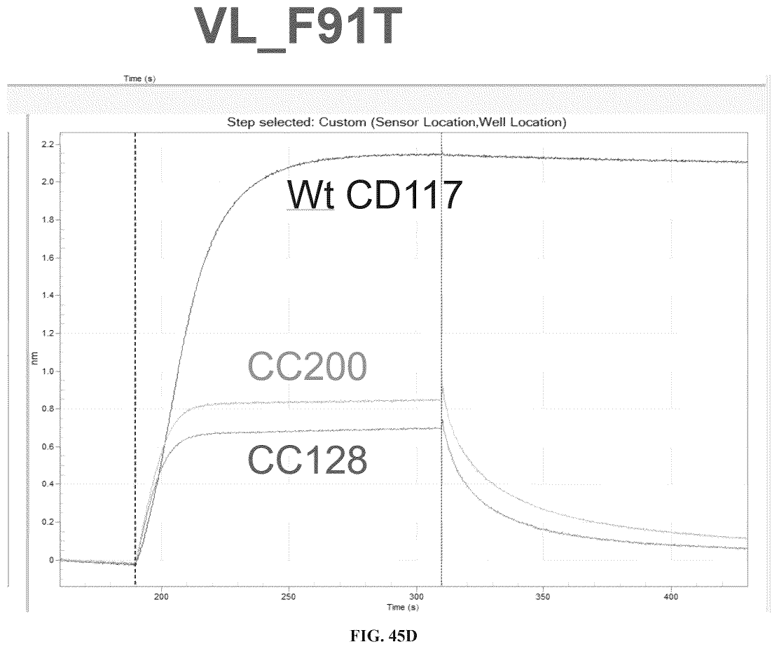

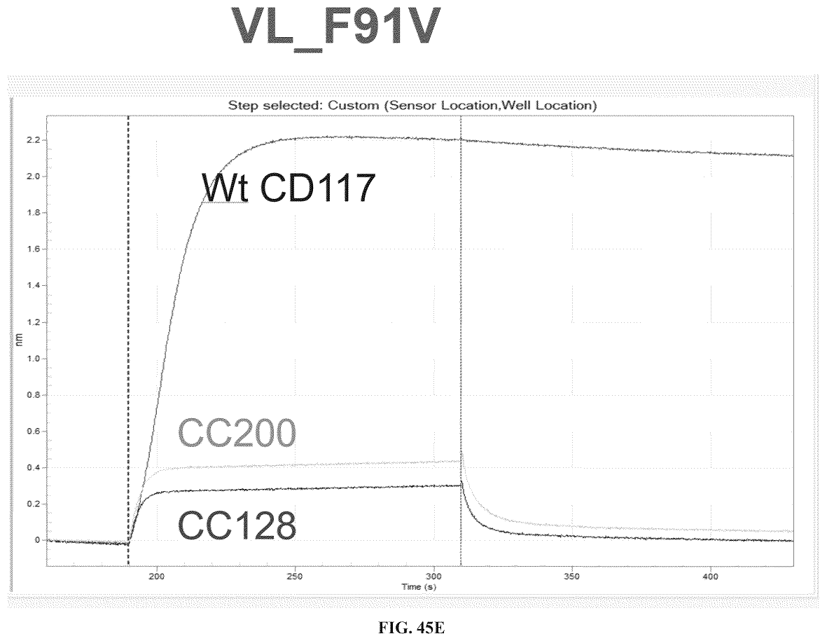

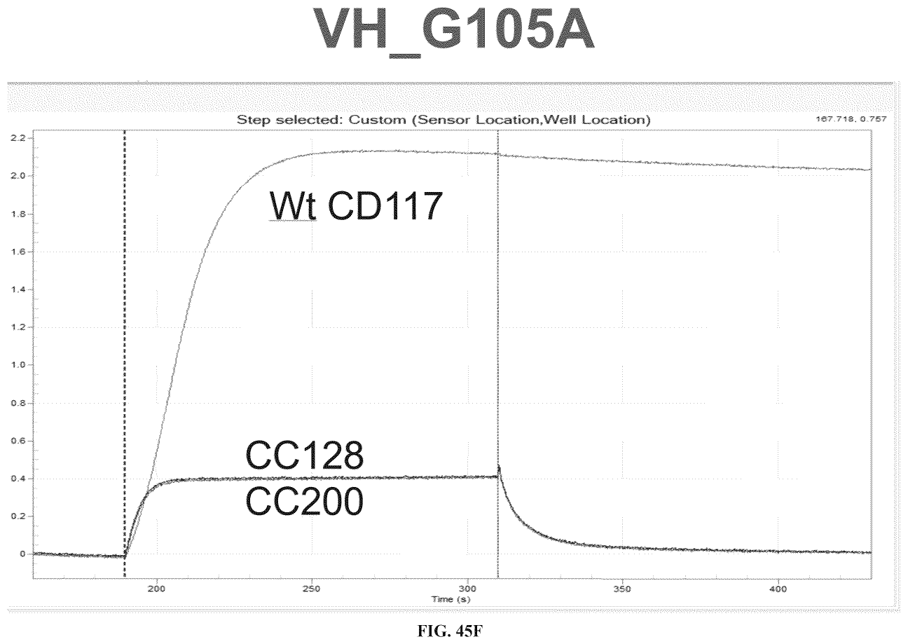

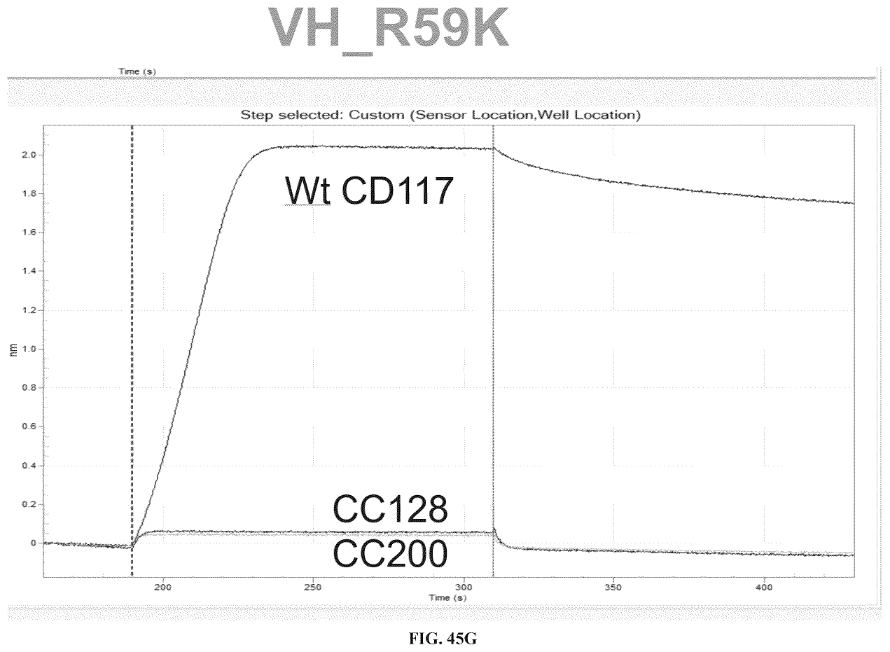

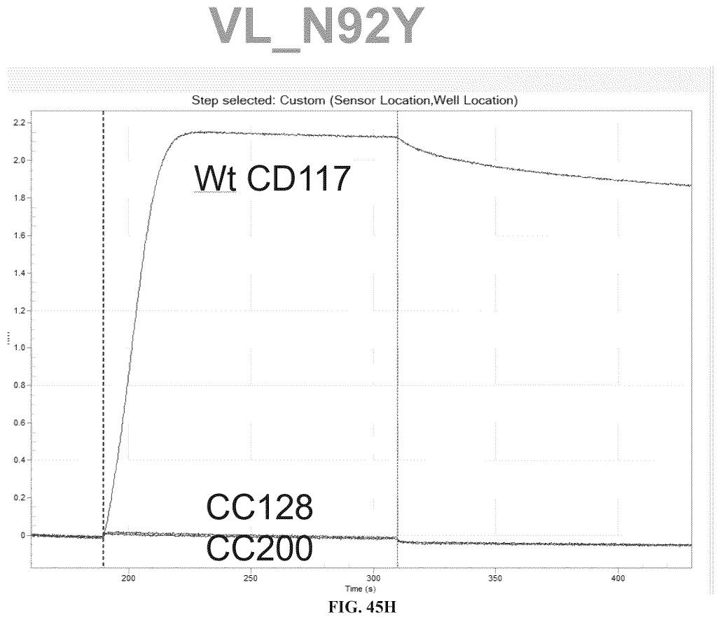

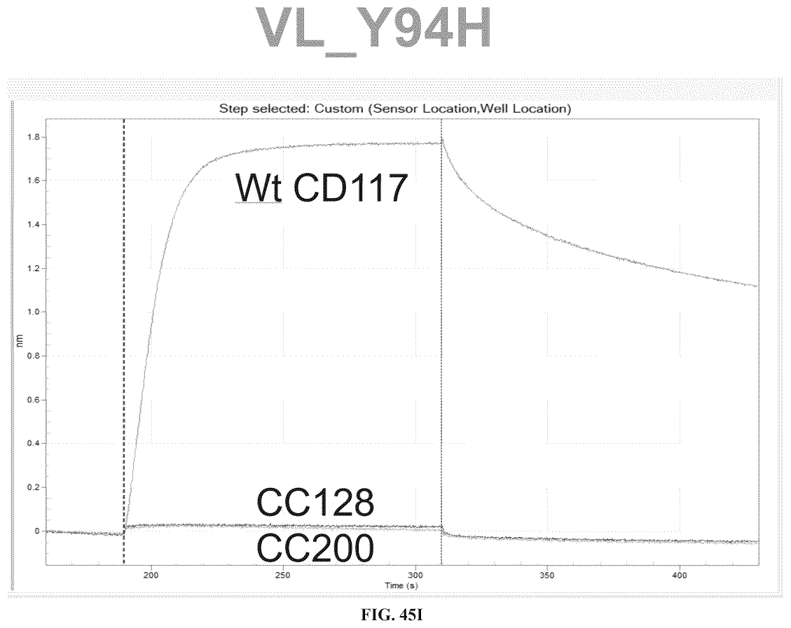

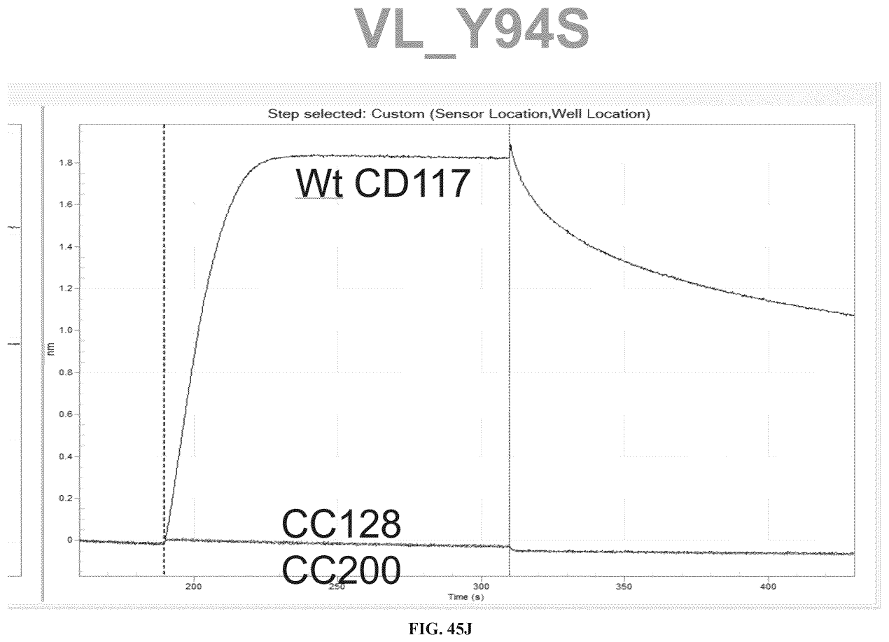

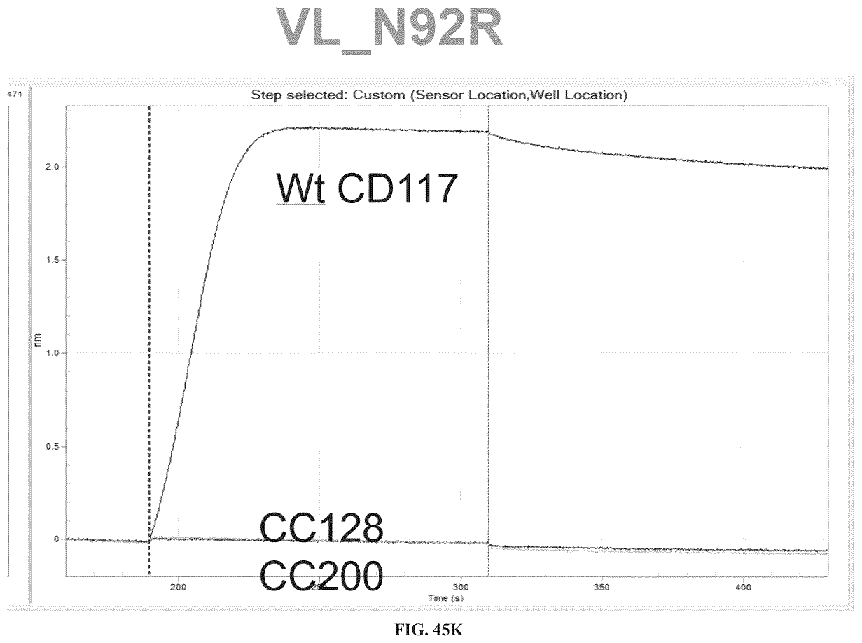

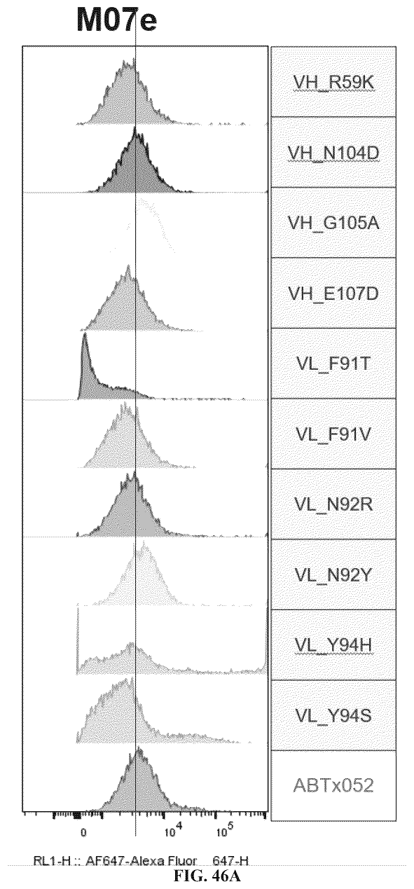

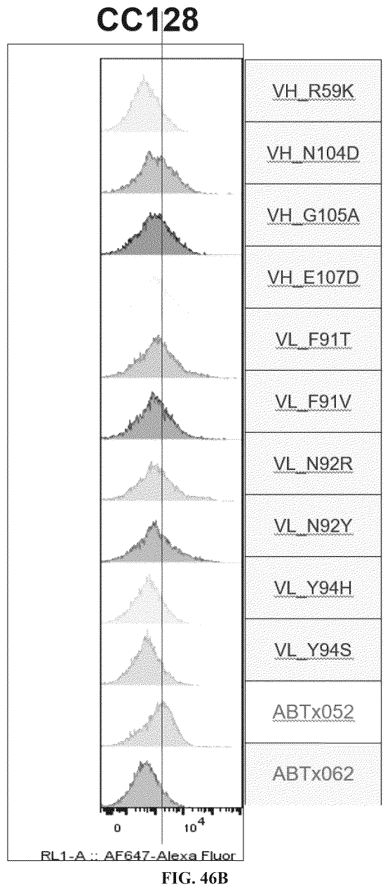

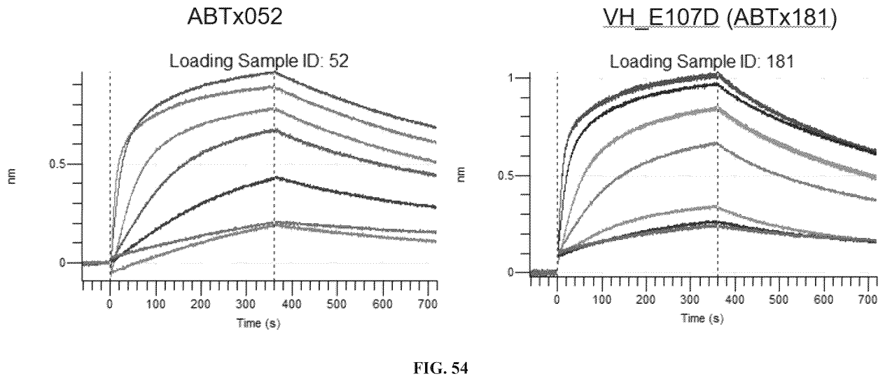

Binding of each of the ABTx052 variants to wild-type CD117 and to the CD117 variants CC128 and CC200 was quantitatively evaluated (see FIGS. 45A-45K and 54) and Tables 18A and 18B). Variants with a slow off-rate for binding to wild-type CD117 were of particular interest on account of the slow off-rate likely corresponding to an increased likelihood of interfering with the binding of stem cell factor (SCF) to CD117. The ABTx052 variant VH_E107D (ABTx181) showed good binding to wild-type CD117 with a slow off-rate and low binding to both CD117 variants CC128 and CC200, which would be advantageous characteristics for use of the antibody in the engineered stem cell antibody-paired evasion (ESCAPE) approaches for engraftment of hematopoietic stem cells (HSCs) provided herein. ABTx052 variant VH_E107D had a K_{D} for binding to CD117 that was comparable to that of ABTx052 (FIG. 54 and Table 18B). Most of the ABTx052 variants evaluated showed binding to M07e cells that expressed wild-type CD117 and reduced binding to M07e cells that expressed the CC128 variant of CD117 (FIGS. 46A and 46B).

TABLE 18A
Summary of binding of the indicated ABTx052 variants to wild-
type CD117 and to the CD117 variants CC128 and CC200.

| Antibody | CD117 | | | Off | | | | Full |
|---|---|---|---|---|---|---|---|---|
| ID | wt | CC128 | CC200 | Rate | K_{D} (M) | kₐ (1/Ms) | k_{dis} (1/s) | R\hat{ }2 |
| VL_F91T | strong | similar | >>control | slow | 1.91E−10 | 5.43E+05 | 1.04E−04 | 0.973 |
| VH_G105A | strong | similar | similar | slow | 2.54E−10 | 6.01E+05 | 1.53E−04 | 0.9759 |
| VL_F91V | strong | <control² | similar | slow | 2.34E−10 | 6.54E+05 | 1.53E−04 | 0.9813 |
| VH_E107D | strong | <<control | <<control | slow | 3.76E−10 | 6.09E+05 | 2.29E−04 | 0.9762 |
| (ABTx181) | | | | | | | | |
| VH_N104D | strong | <<control | similar | slow | 4.95E−10 | 6.91E+05 | 3.42E−04 | 0.9677 |
| VL_N92R | strong | NO³ | NO | medium | 6.83E−10 | 6.35E+05 | 4.34E−04 | 0.9747 |
| VL_N92Y | strong_F⁴ | NO | NO | medium | 8.41E−10 | 7.55E+05 | 6.35E−04 | 0.9764 |
| VH_R59K | strong_F | NO | NO | medium | 9.86E−10 | 7.23E+05 | 7.13E−04 | 0.9701 |
| VL_Y94H | Strong_F | NO | NO | Fast | 3.16E−09 | 5.63E+05 | 1.78E−03 | 0.9791 |
| VL_Y94S | strong_F | NO | NO | Fast | 3.38E−09 | 6.43E+05 | 2.18E−03 | 0.9667 |
| ABTx135 | strong | control | control | slow | 2.60E−10 | 6.77E+05 | 1.76E−04 | 0.9733 |
| VL_S93G | strong | strong | strong | slow | 7.16E−11 | 6.71E+05 | 4.81E−05 | 0.984 |

²“<<control,” “<control,” and “>>control” indicate much lower binding than ABTx135, which had binding characteristics similar to ABTx052, less binding than ABTx135, and much greater binding than ABTx135, respectively.
³“NO” indicates no measured binding.
⁴“strong F” indicates strong binding with a medium to fast off rate

TABLE 18B
Summary of binding of the indicated ABTx052 variants to wild-
type CD117 and to the CD117 variants CC128 and CC200.

| | | | | Full |
|---|---|---|---|---|
| Ab ID | K_{D}(M) | Kₐ (1/Ms) | K_{dis} (1/s) | R\hat{ }2 |
| ABTx052 | 9.298E−09 | 1.272E05 | 1.182E−03 | 0.9966 |
| VH_E107D | 9.324E−09 | 1.772E05 | 1.652E−03 | 0.9838 |
| (ABTx181) | | | | |

As described in Table 18, the K_{D} (equilibrium dissociation constant), kₐ (equilibrium association constant), k_{dis} (equilibrium dissociation rate constant), and Full R\hat{ }2 values characterize binding of the indicated ABTx052 variant or ABTx135 to wild-type CD117. R\hat{ }2 ranged from 0-1, where a value of 1 indicated that predictions calculated using the K_{D}, Kₐ, and Kdis constants exactly predicted the observed values. A “slow off rate” is indicated by a k_{dis} of less than about 4E-04. A “medium” off rate is indicated by a k_{dis} of between about 4E-04 and 1E-03. A “fast” off rate is indicated by a k_{dis} of less than about 1E-03.

### Example 6: mAb-7 Treatment Induced Hematopoietic Stem and Progenitor Cell (HSPC) Apoptosis In Vitro

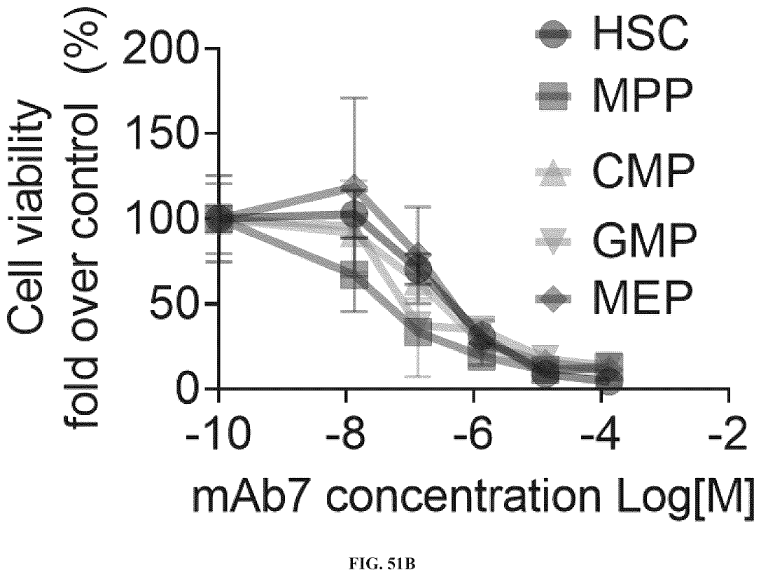

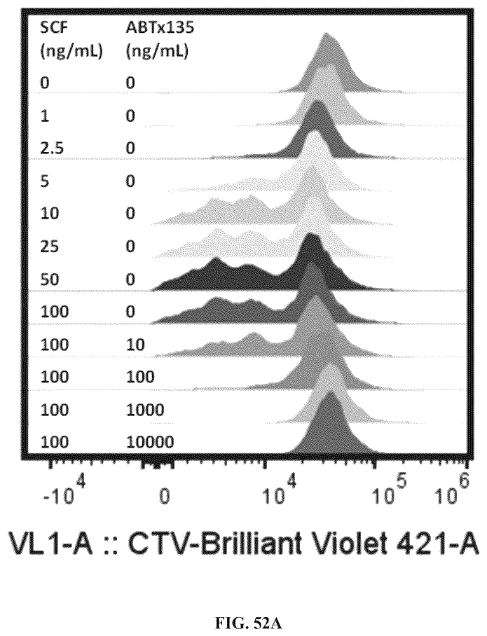

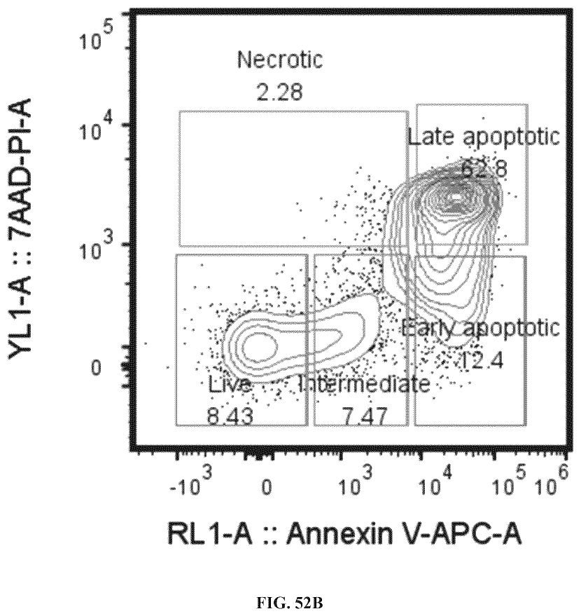

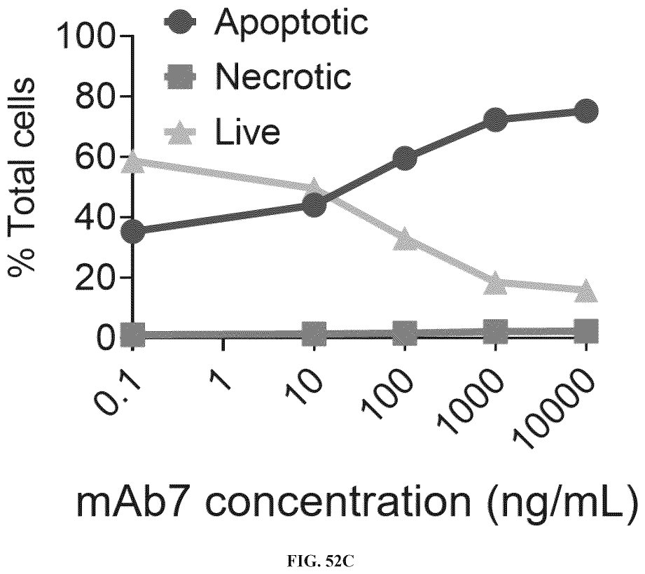

Experiments were undertaken to demonstrate that treatment with ABTx135 induced apoptosis of HSPC cells. Healthy human mobilized peripheral blood derived CD34+HSPCs were thawed and loaded with cytoplasmic dye cell-trace violet. A portion of the cells were then cultured in the presence of varying concentrations of stem cell factor (SCF) (0-100 ng/ml). The other portion of the cells were cultured in the presence of 100 ng/mL SCF and varying concentrations (10-10000 ng/mL) of ABTx135. The cells were cultured for 5 days at 37° C. using a tissue culture incubator. At Day 5, the cells were co-stained with Annexin V and 7-AAD and interrogated using flow cytometry (FIGS. 52A-52C). ABTx135 treatment induced apoptosis of the cells (FIG. 52C).

### Example 7: ABTx135 Engraftment of CC128 Base Edited Hematopoietic Stem and Progenitor Cells (HSPCs) Post Anti-CD117 mAb-Based Conditioning

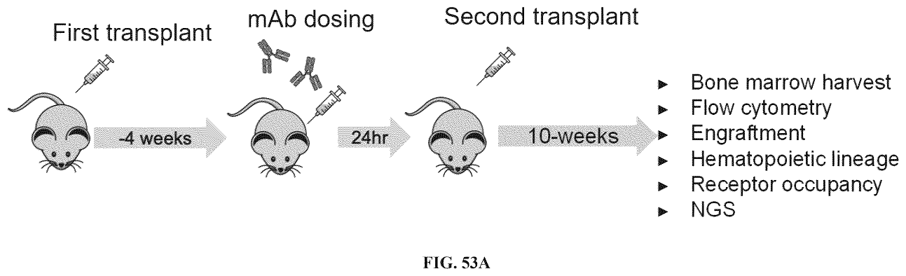

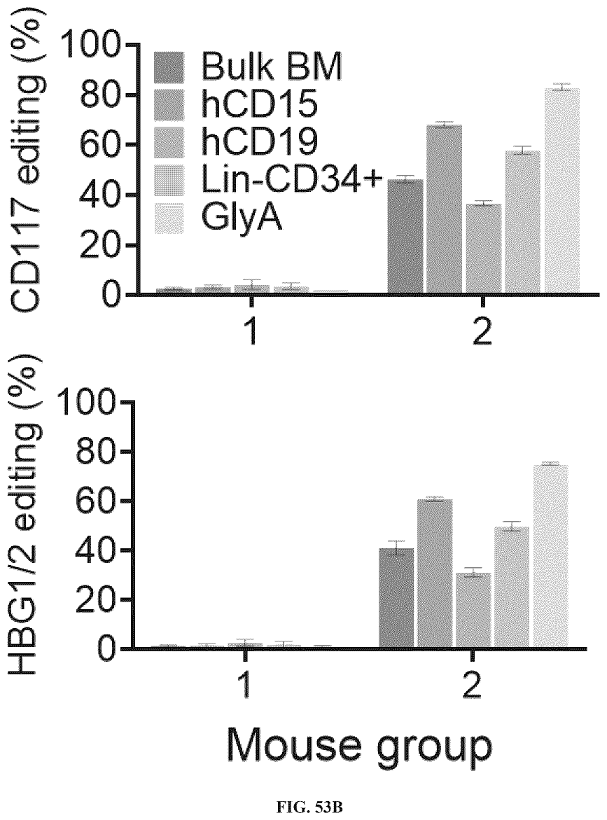

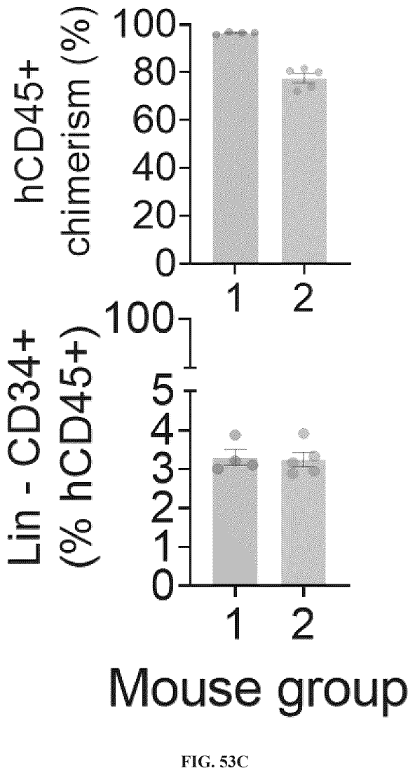

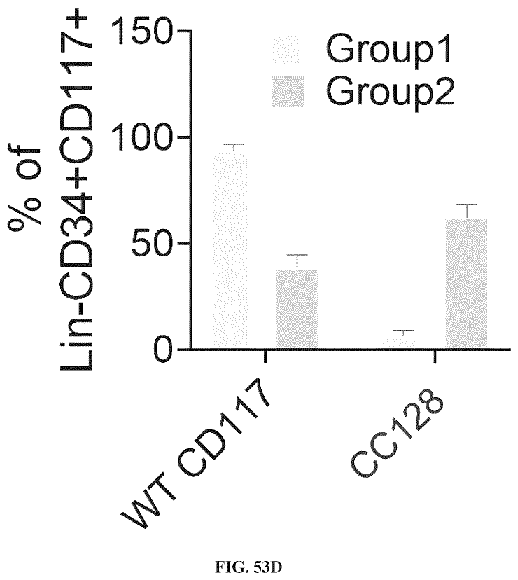

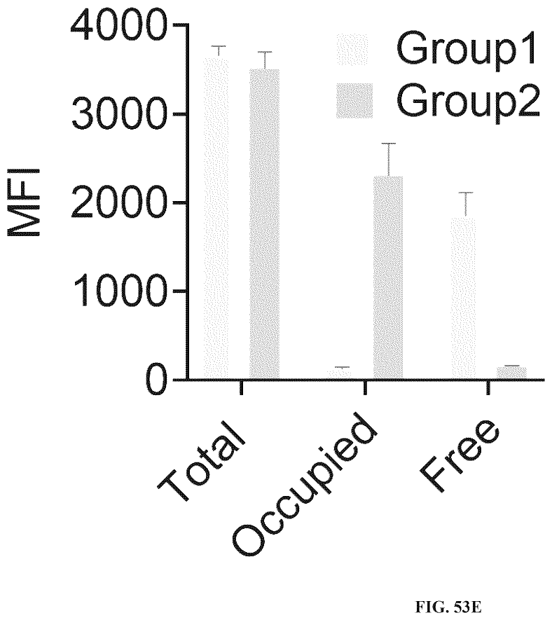

Experiments were undertaken to demonstrate the engraftment of base edited hematopoietic stem cells (HSCs) in mice previously treated with an anti-CD117 monoclonal antibody. As shown in FIG. 53A, NBSGW mice were first transplanted with 1e6 wild type (unedited) healthy human CD34+HSPCs. 4 weeks post transplantation animals were treated with either isotype control antibody or ABTx135 (see Table 19). 24 hours after antibody dosing, a second transplant was performed with 1E+6 cells multiplex edited using the base editor ABE8.8 in combination with sgRNA CC128 (i.e., gRNA434) and sgRNA_015 (HBG1/2-114 edit). 10 weeks after the second transplant, the mice were sacrificed, and bone marrow was harvested. Table 19 provides details relating to mAb treatment and transplantation for the mouse groups. A part of the bulk bone marrow from each mouse group was subjected to genomic DNA isolation and subsequent next generation sequencing analysis. In addition, human CD15+, Human CD19+, and Lin-human CD34+, and GlyA+ cells were isolated from the bone marrow using flow assisted sorting. Genomic DNA was isolated from the sorted cells and was subjected to next generation sequencing. FIG. 53B shows levels of on-target editing in the sorted cell populations within the CD117 (top panel) and HBG1/2 (bottom panel) sites targeted for base editing. The mouse bone marrow was interrogated using flow cytometry to determine human engraftment and multi-lineage reconstitution. FIG. 53C show human CD45+ chimerism (top panel) and human Lin−CD34+ engraftment (bottom panel) within mouse bone marrow. FIG. 53D shows frequencies of unedited and CD117-edited cells within human Lin−CD34+CD117+ subsets within bone marrow. FIG. 53E shows levels of total CD117 receptor on the surface of human Lin−CD34+CD117+ cells, mAb-occupied CD117 receptor on the surface of unedited Lin−CD34+CD117+ cells, and free CD117 receptors within Lin−CD34+CD117+ cells. These data demonstrate the successful engraftment of the multiplex base edited HSCs in the mice previously treated using an anti-CD117 monoclonal antibody for conditioning.

TABLE 19
Transplant and dosing groups.

| | First | Second | |
|---|---|---|---|
| | transplant | transplant | mAb |
| Group | (“Host”) | (“Graft”) | treatment |
| 1 | Unedited | CD117/HBG1/2- | Isotype |
| | | Multiplex | |
| 2 | Unedited | CD117/HBG1/2- | mAb-7 |
| | | Multiplex | |

### Example 8: Engineering of Antibodies Suitable for Use in an Engineered Stem Cell Antibody-Paired Evasion (ESCAPE) Approach for Engraftment of Hematopoietic Stem Cells (HSCs)

Experiments were undertaken to identify antibodies suitable for use in the ESCAPE approach for engraftment of HSCs. As a first step to this end, the antibodies described in Table were expressed in Chinese hamster ovary (CHO) cells and purified to yield about 3 mg of each antibody from a 20 mL cell culture. A monomer purity of >95% was confirmed using SDS PAGE and SEC-HPLC. The antibodies were characterized as described below.

TABLE 20
Summary of antibody amino acid sequences.

| Antibody | Mutations relative to ABTx052 in the heavy chain variable region (VH) |
|---|---|
| Identifier | and light chain variable region (VL). |
| ABTx248 | ABTx052 VH-N104D_VH-E107D VL |
| ABTx249 | ABTx052 VL-F91L_VL-S93G_VH-N104D |
| ABTx250 | ABTx052 VH-R59K_VH-N104D VL |
| ABTx251 | ABTx052 VL-N92Y_VH-N104D |
| ABTx273 | ABTx052 VL-N92R_VH-N104D |
| ABTx252 | ABTx052 VL-Y94H_VH-N104D |
| ABTx253 | ABTx052 VL-N92S_VH-N104D |
| ABTx254 | ABTx052 VL-F91T_VL-Y94H_VH-R59K_VH-N104D_VH-E107D |
| ABTx255 | ABTx052 VL-F91V_VL-N92R_VH-R59K_VH-N104D |
| ABTx256 | ABTx052 VL-F91T_VL-N92Y_VH-N104D_VH-G105A |
| ABTx257 | ABTx052 VL-F91T_VH-R59K_VH-N104D_VH-G105A_VH-E107D |
| ABTx258 | ABTx052 VL-F91V_VL-N92R_VH-R59K_VH-N104D_VH-G105A |
| ABTx259 | ABTx052 VL-F91T_VL-N92R_VH-R59K_VH-N104D |
| ABTx260 | ABTx052 VL-F91V_VL-Y94H_VH-R59K_VH-N104D_VH-E107D |
| ABTx261 | ABTx052 VL-F91T_VL-N92R_VH-R59K_VH-N104D_VH-G105A |
| ABTx262 | ABTx052 VL-N92R_VL-Y94S_VH-N104D_VH-G105A |
| ABTx263 | ABTx052 VL-F91V_VL-N92Y_VH-R59K_VH-N104D_VH-E107D |
| ABTx264 | ABTx052 VL-F91T_VL-N92Y_VH-R59K_VH-N104D_VH-G105A |
| ABTx274 | ABTx052 VL-F91T_VH-R59K_VH-N104D |
| ABTx265 | ABTx052 VL-F91T_VL-N92R_VH-R59K_VH-N104D_VH-E107D |
| ABTx266 | ABTx052 VL-F91V_VL-N92R_VL-Y94H_VH-N104D_VH-G105A |
| ABTx267 | ABTx052 VL-F91T_VL-Y94S_VH-R59K_VH-N104D_VH-G105A_VH- |
| | E107D |
| ABTx268 | ABTx052 VL-F91V_VH-R59K_VH-N104D |
| ABTx269 | ABTx052 VL-F91V_VL-N92Y_VH-R59K_VH-N104D |
| ABTx270 | ABTx052 VL-F91V_VL-N92R_VH-N104D_VH-G105A |
| ABTx271 | ABTx052 VL-F91T_VH-N104D_VH-G105A_VH-E107D |
| ABTx272 | ABTx052 VL-F91V_VL-N92R_VH-R59K_VH-N104D_VH-E107D |

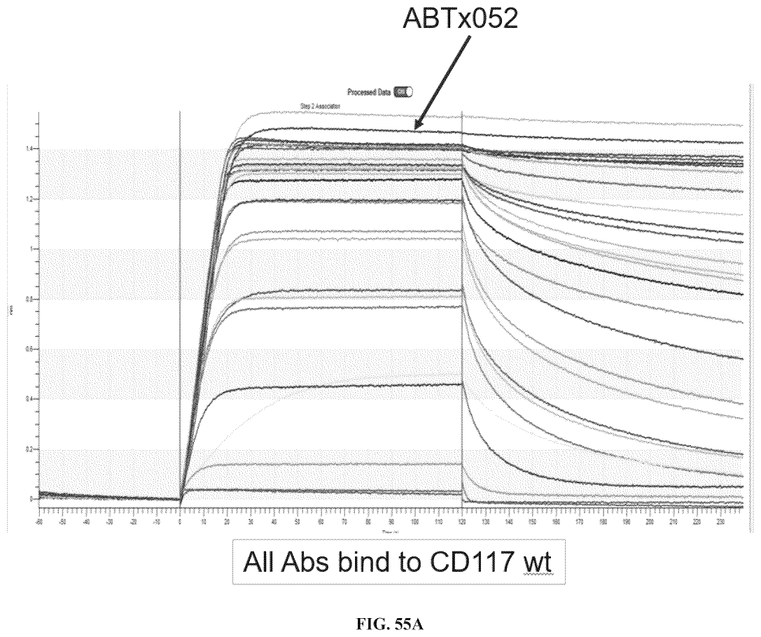

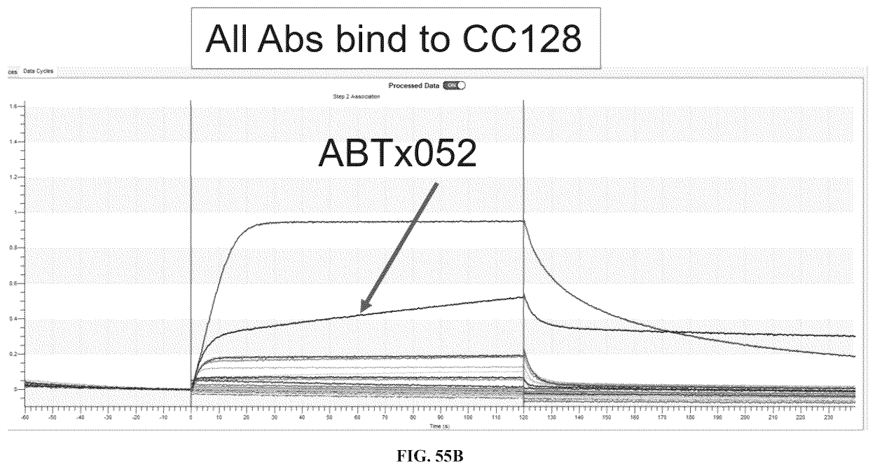

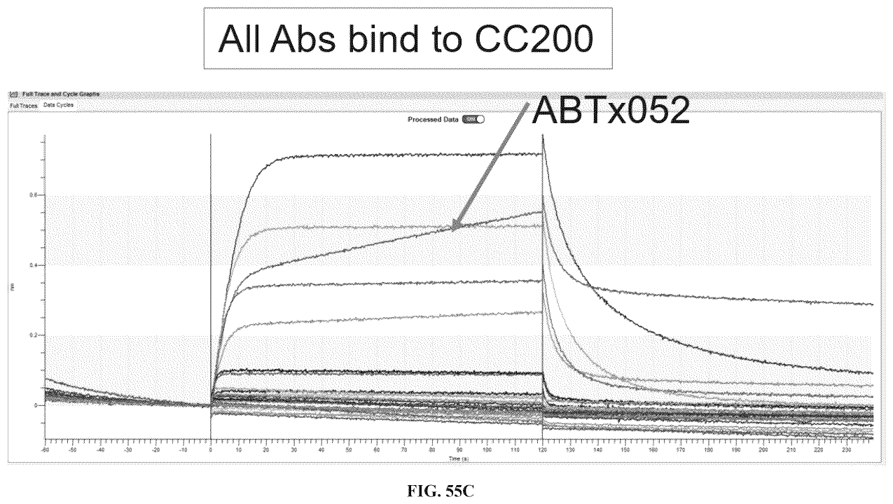

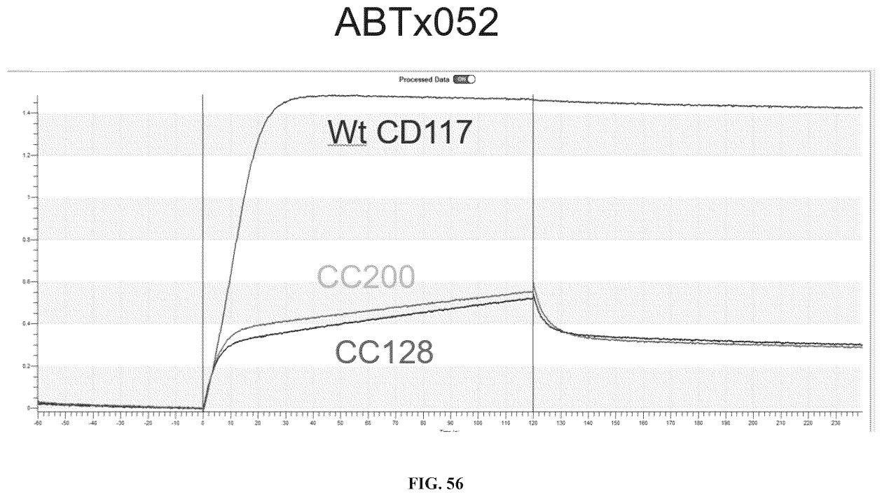

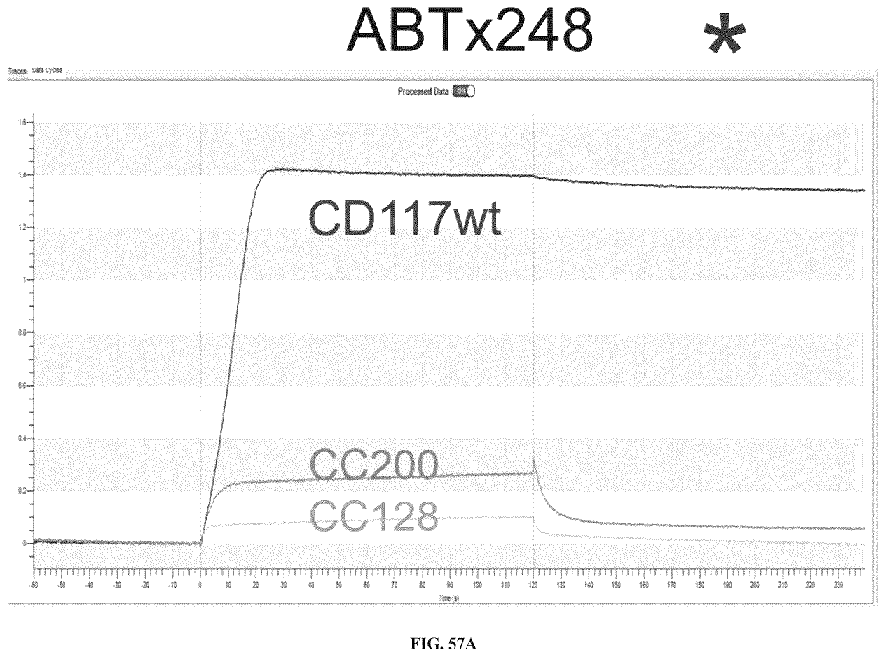

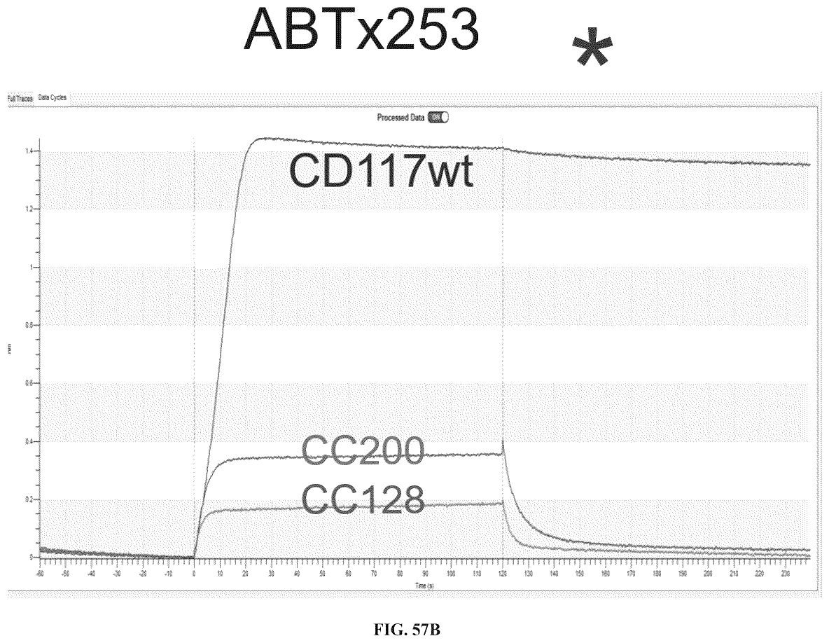

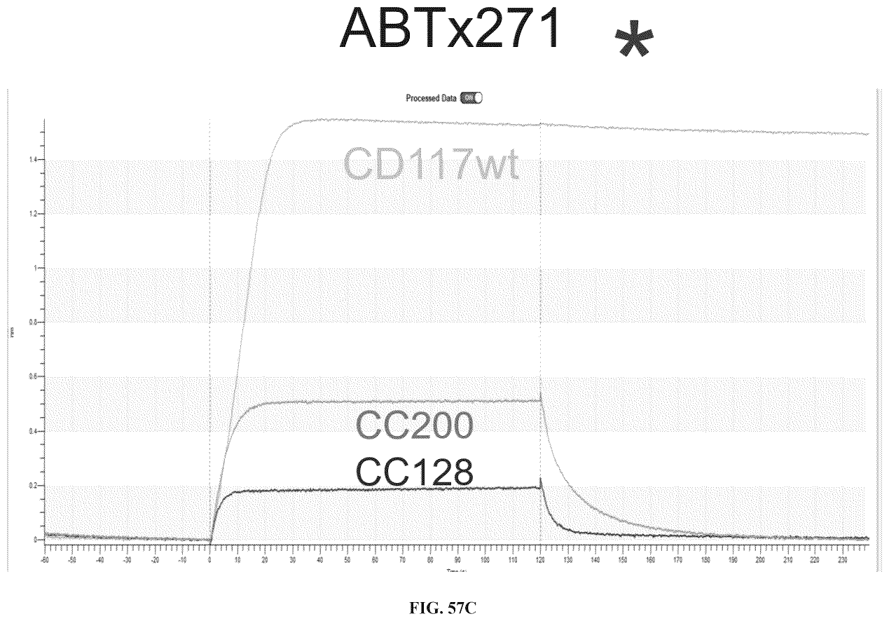

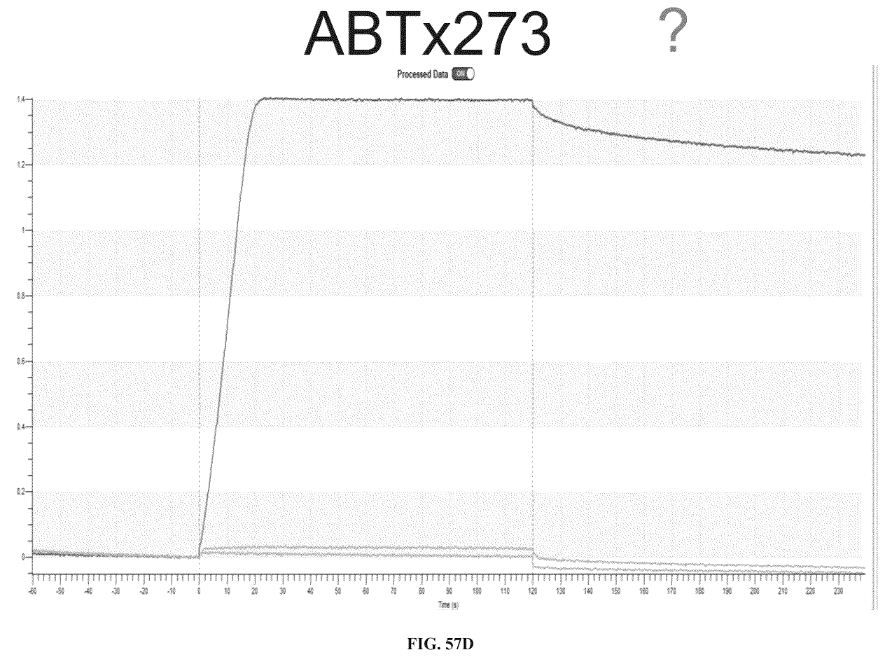

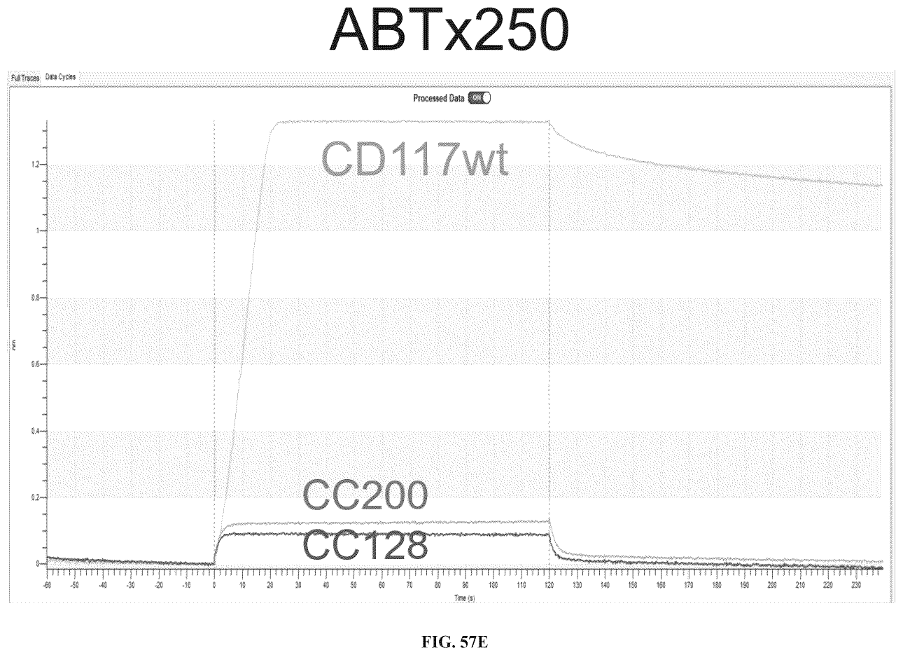

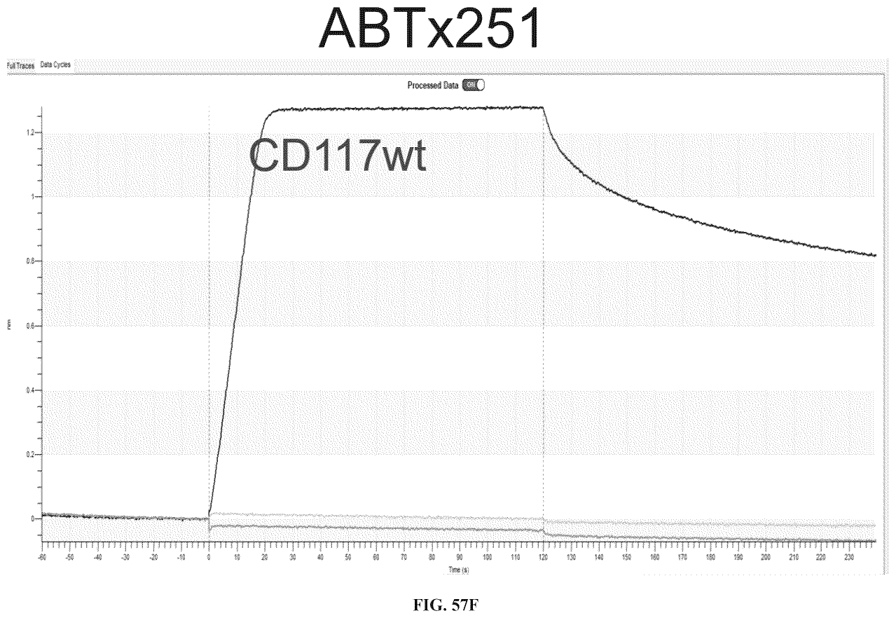

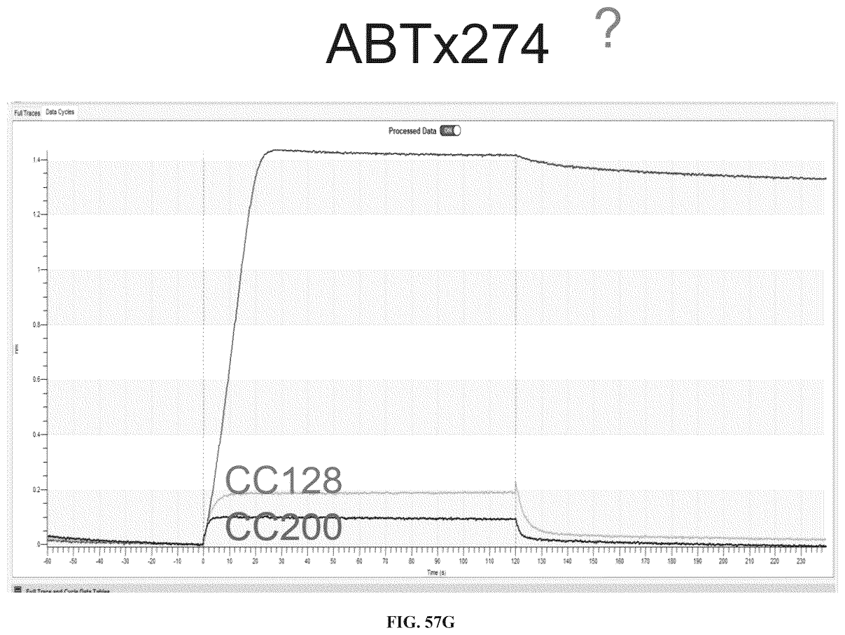

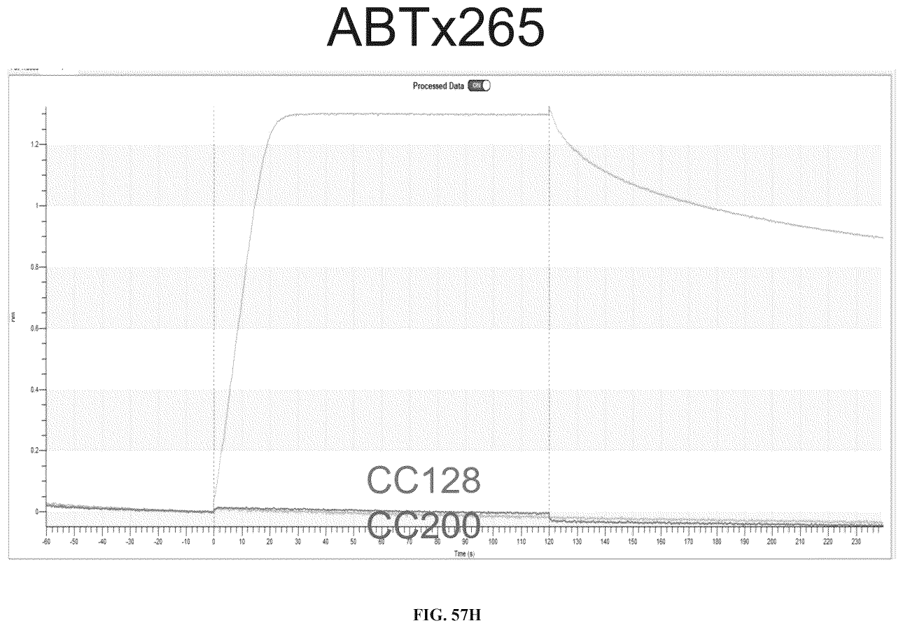

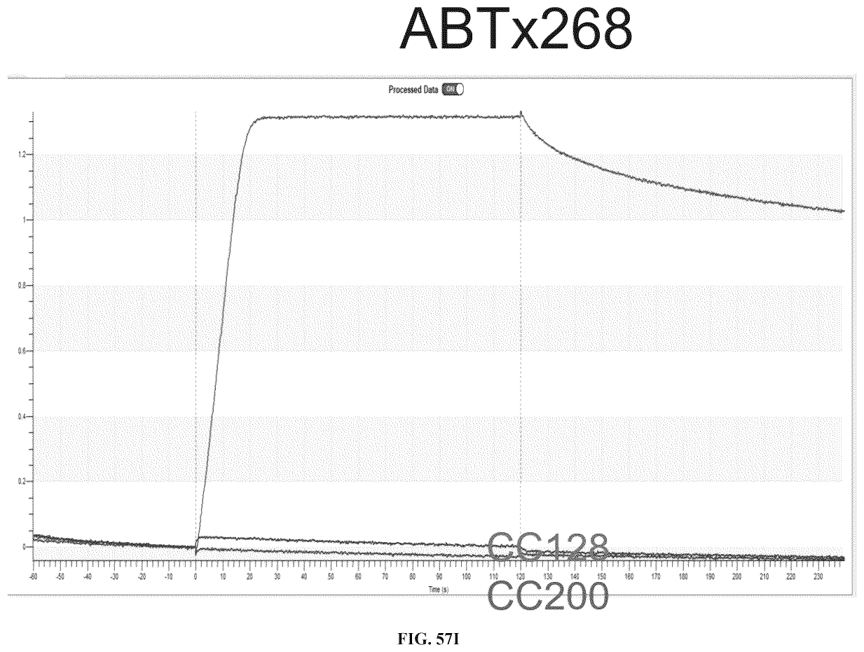

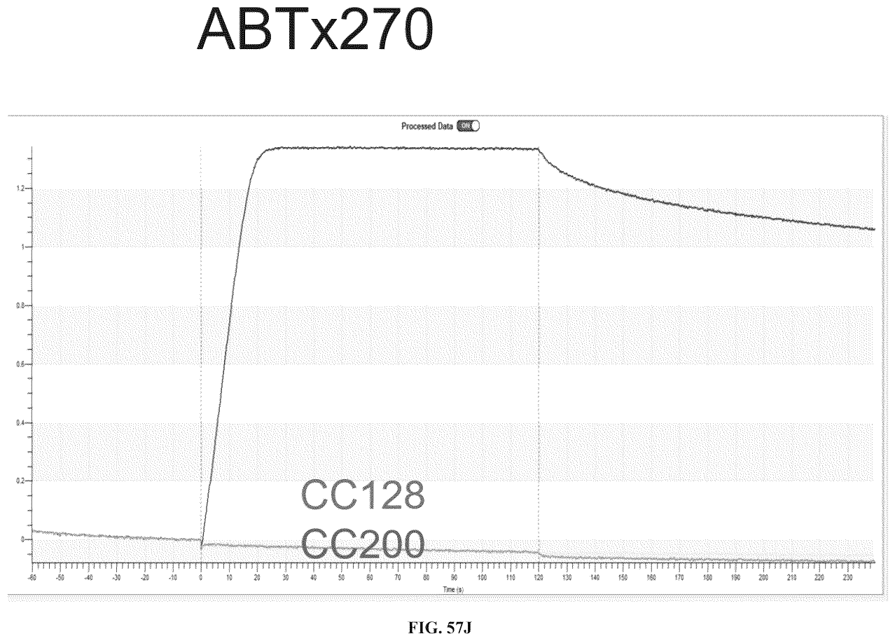

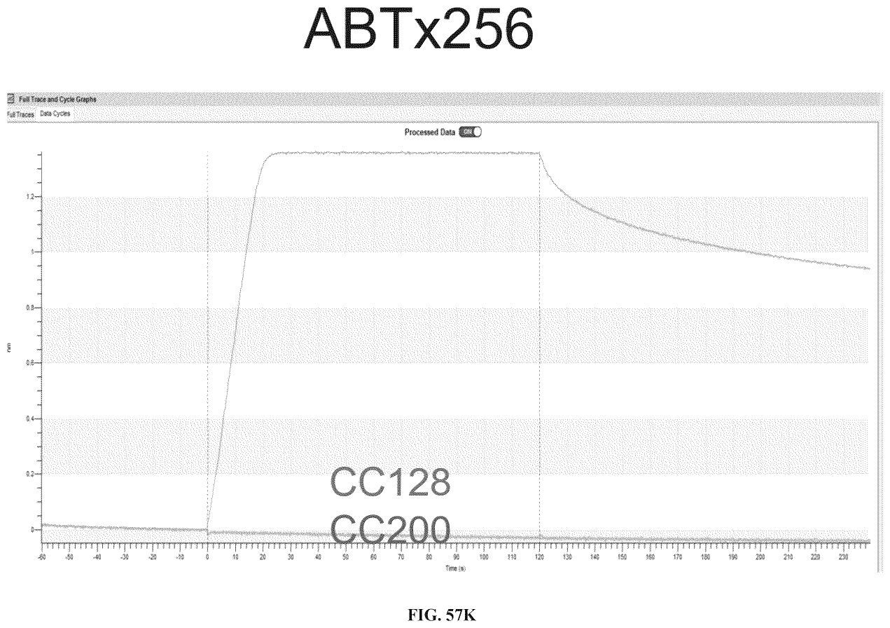

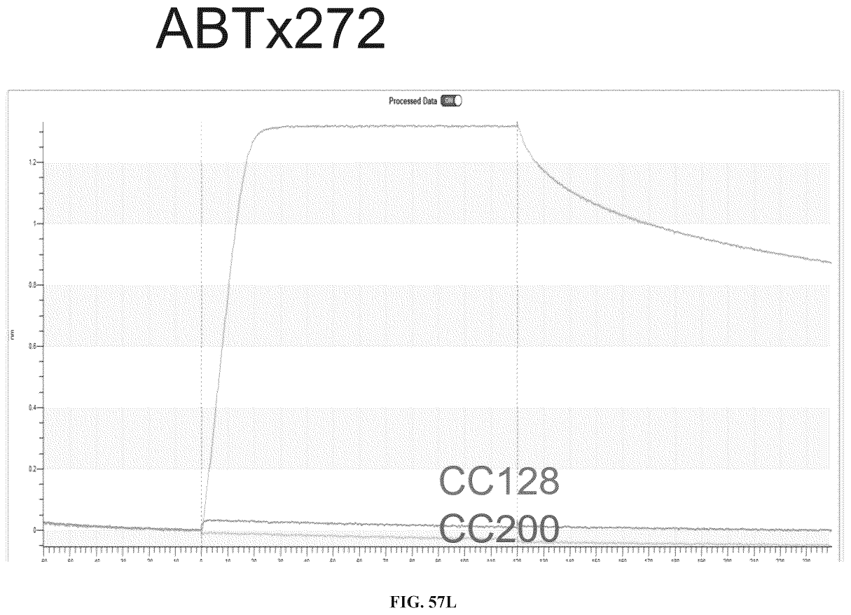

Experiments were undertaken to evaluate for each of the antibodies listed in Table 20 the binding affinity for wild-type CD117 (FIG. 55A), to the CC128 variant of CD117 (FIG. 55B), and to the CC200 variant of CD117 (FIG. 55C) (see also FIGS. 57A-57L). The CC200 and CC128 variants of CD117 were prepared by base editing cells using the guide CC200 or CC128, respectively, and an adenosine deaminase base editor (ABE). Binding affinities for ABTx052 were measured for comparison (FIG. 56). The measured antibody binding affinities are summarized in Table 21. Twelve (12) of the antibodies had strong binding affinity for wt CD117 and lower/same binding affinity to the CC128 and CC200 variants of CD117 (FIGS. 57A-57L).

TABLE 21
Summary of antibody binding affinities for wt
CD117 and the CD117 variants CC128 and CC200.

| | wt | | |
|---|---|---|---|
| Ab ID | CD117 | CC128 | CC200 |
| ABTx248 | Y | N | S |
| ABTx249 | Y | H | H |
| ABTx250 | Y | L | L |
| ABTx251 | Y | N | N |
| ABTx273 | Y | N | N |
| ABTx252 | N | L/N | L/N |
| ABTx253 | Y | L | S |
| ABTx254 | L/N | L/N | L/N |
| ABTx255 | L/N | L/N | L/N |
| ABTx256 | Y | N | N |
| ABTx257 | Y/L | L | L |
| ABTx258 | L/N | N | N |
| ABTx259 | L/N | N | N |
| ABTx260 | L/N | N | N |
| ABTx261 | L/N | N | N |
| ABTx262 | L/N | N | N |
| ABTx263 | L/N | N | N |
| ABTx264 | L/N | N | N |
| ABTx274 | Y | L | L |
| ABTx265 | Y | N | N |
| ABTx266 | L/N | L | L |
| ABTx267 | L/N | N | N |
| ABTx268 | Y | N | N |
| ABTx269 | L/N | N | N |
| ABTx270 | Y | N | N |
| ABTx271 | Y | L | S |
| ABTx272 | Y | N | N |
| ABTx052 | Y | L | L |

“N” indicates no binding, “Y” indicates binding, “H” indicates high binding, “L” indicates low binding, “S” indicates strong binding, and “L/N” indicates low-to-no binding.

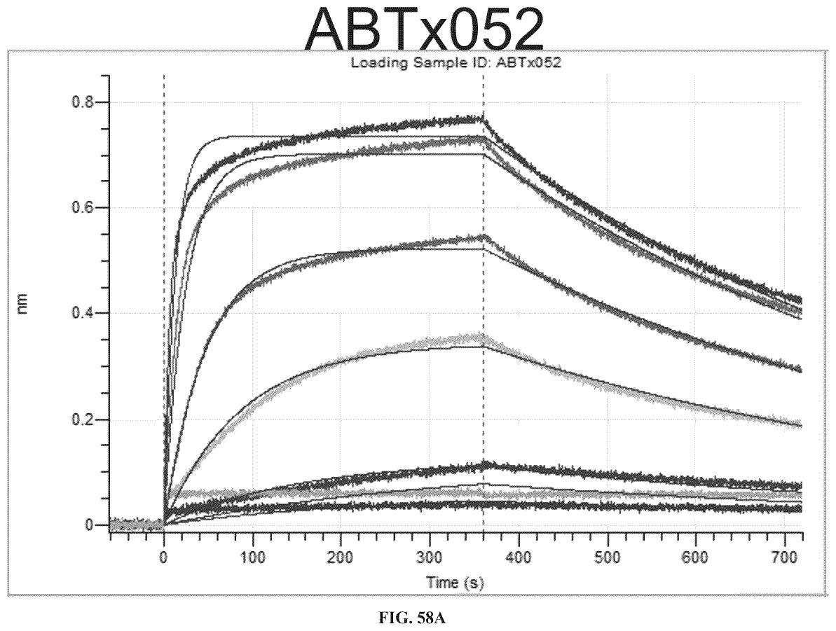

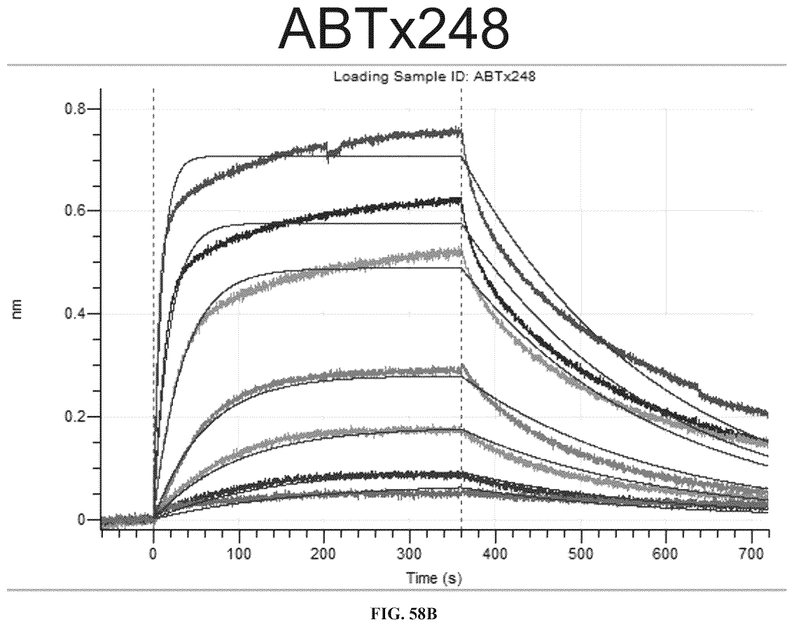

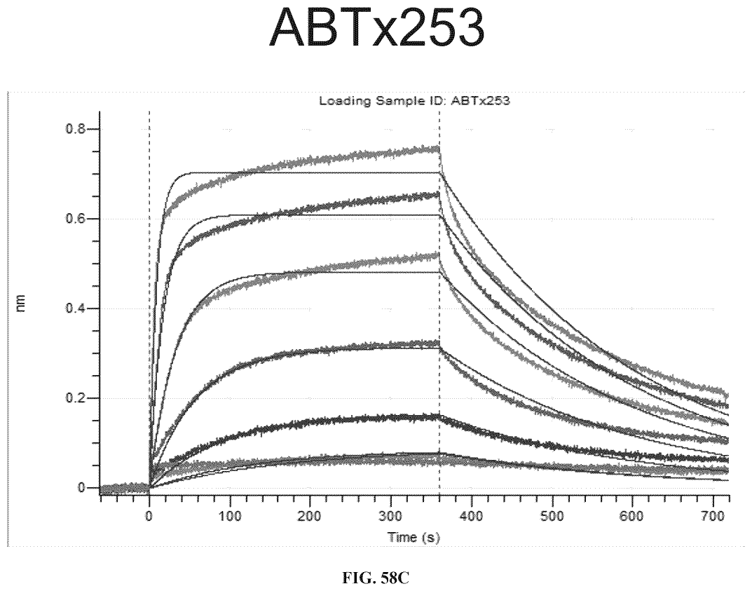

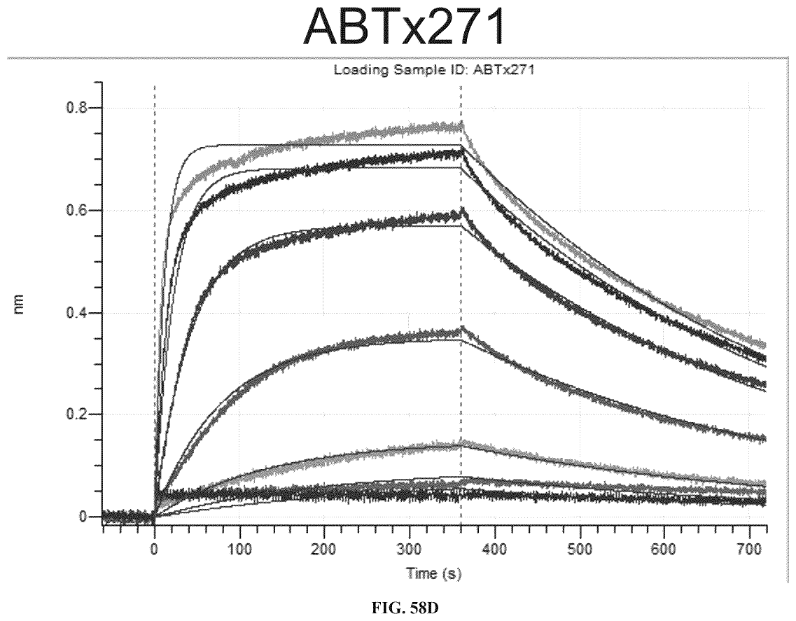

Experiments were undertaken to quantify the wild-type CD117 binding kinetics for the following antibodies: ABTx052, ABTx248, ABTx253, and ABTx271 (FIGS. 58A-58D). Table 22 provides a quantitative summary of the binding observed for each of the antibodies. Each of ABTx248, ABTx253, and ABTx271 had lower KDs compared to ABTx052.

TABLE 22
Summary of binding of the indicated
ABTx052 variants to wild-type CD117.

| | K_{D} | kₐ | k_{dis} | Full |
|---|---|---|---|---|
| | (M) | (1/Ms) | (1/s) | R\hat{ }2 |
| ABTx052 | 7.22E−09 | 2.28E+05 | 1.65E−03 | 0.9963 |
| ABTx248 | 1.53E−08 | 2.83E+05 | 4.32E−03 | 0.991 |
| ABTx253 | 1.31E−08 | 3.12E+05 | 4.08E−03 | 0.9899 |
| ABTx271 | 9.53E−09 | 2.46E+05 | 2.34E−03 | 0.9961 |

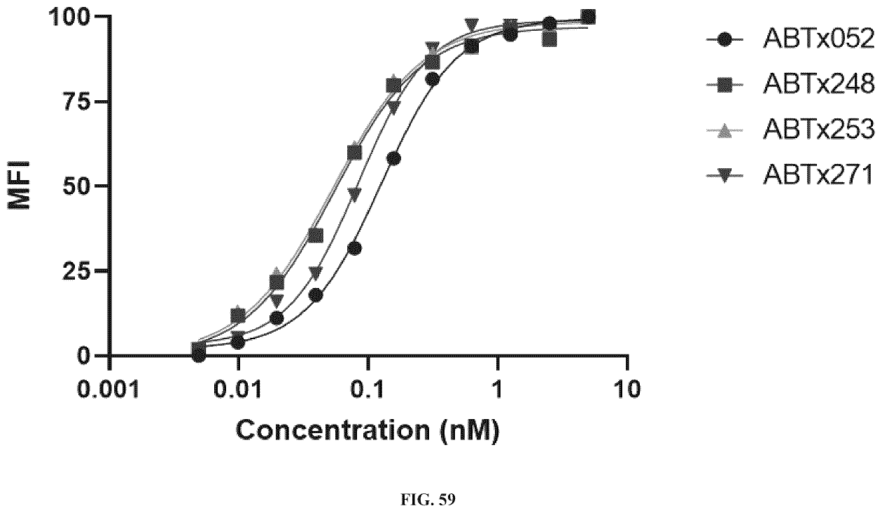

Experiments were undertaken to determine EC50 (50% effective concentration) values on M07e cells for each of the following antibodies: ABTx052, ABTx248, ABTx253, and ABTx271 (FIG. 59 and Table 23). Each of ABTx248, ABTx253, and ABTx271 had a lower EC50 value than ABTx052 on M07e cells.

TABLE 23
EC₅₀ values for the indicated antibodies on M07e cells.

| Ab ID | EC₅₀ (nM) |
|---|---|
| ABTx052 | 0.1256 |
| ABTx248 | 0.0541 |
| ABTx253 | 0.0529 |
| ABTx271 | 0.0836 |

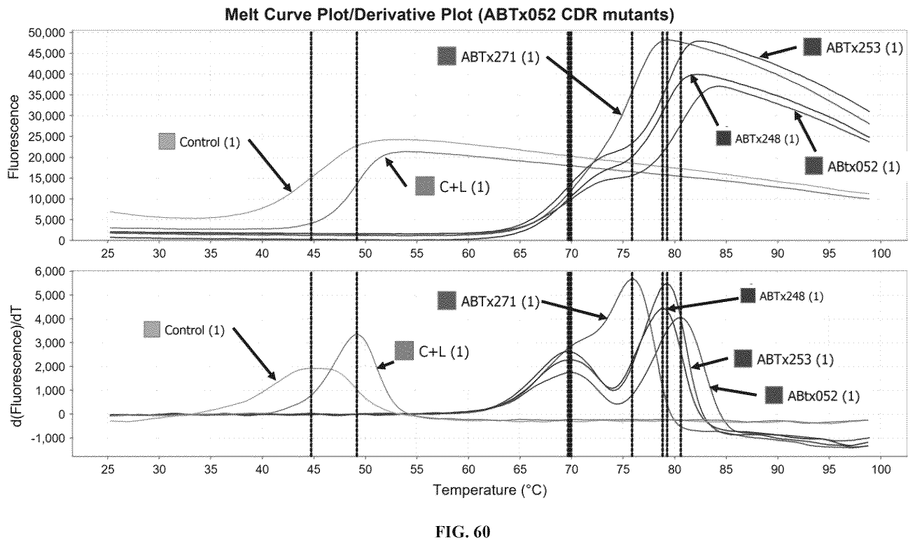

Experiments were then undertaken to calculate melting temperatures (Tms) for each of the following antibodies: ABTx052, ABTx248, ABTx253, and ABTx271 (FIG. 60 and Table 24). The antibodies ABTx248, ABTx253, and ABTx271 each had melting temperatures that were comparable to those of ABTx052 (Table 24).

TABLE 24
Tms values for the indicated antibodies.

| Ab ID | Tm1(° C.) | Tm2 (° C.) |
|---|---|---|
| ABTx052 | 69.96 | 80.58 |
| ABTx248 | 69.82 | 78.81 |
| ABTx253 | 69.67 | 79.26 |
| ABTx271 | 75.86 | |

The polyspecificity of ABTx248, ABTx253, and ABTx271 was evaluated (FIG. 61) and it was determined that each of the antibodies had low polyspecificity.

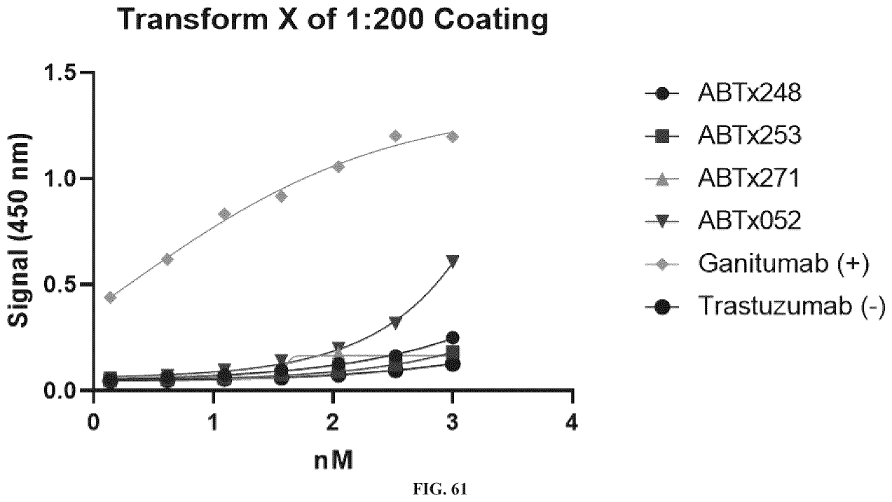

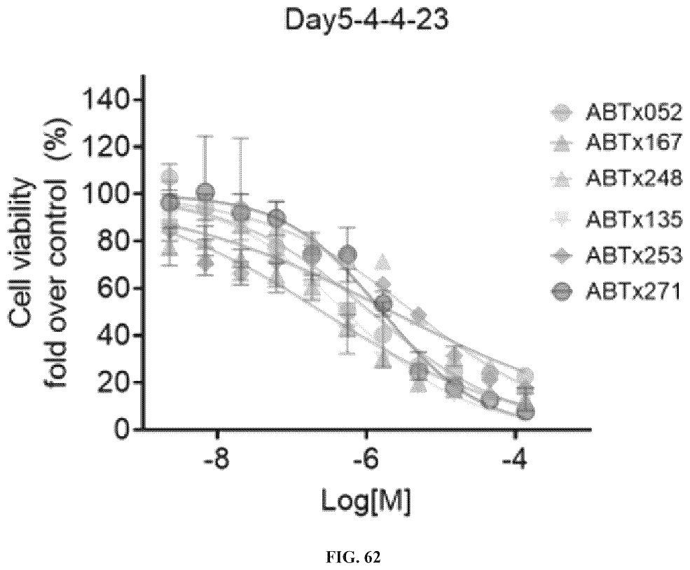

An experiment was undertaken to determine the viability of hematopoietic stem cells (HSCs) treated with increasing concentrations of the antibodies ABTx167, ABTx248, ABTx135, ABTx253, and ABTx271 (FIG. 62 and Table 25). Each of the antibodies had IC50 values that were similar to or lower than that for ABTx052 (Table 25).

TABLE 25
Summary of IC50 values for the indicated antibodies when
used to treat unedited hematopoietic stem cells in vitro.

| Samples | Background | IC50 [M] |
|---|---|---|
| ABTx052 | — | 1.138e−006 |
| ABTx135 | ABTx052_LALADA | 5.337e−007 |
| ABTx167 | ABTx052_LALADA_YTE | 2.556e−007 |
| ABTx248 | ABTx052 mutant | 4.313e−006 |
| ABTx253 | ABTx052 mutant | 2.210e−006 |
| ABTx271 | ABTx052 mutant | 1.640e−006 |

### Example 9: Transplant of Multiplex Base Edited Hematopoietic Stem Cells (HSCs) into Humanized NBSGW Mice

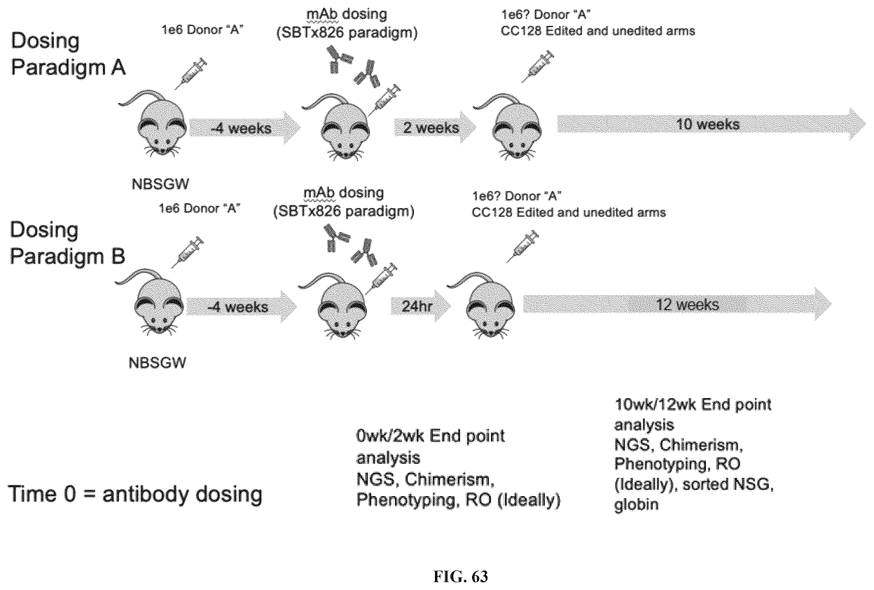

Experiments were undertaken to demonstrate the engraftment of base edited hematopoietic stem cells (HSCs) in mice previously treated with an anti-CD117 monoclonal antibody. As shown in FIG. 63, NBSGW mice were treated according to either “Dosing Paradigm A” or “Dosing Paradigm B.” In each dosing paradigm, mice were first transplanted with 1e6 wild type (unedited) healthy human CD34+HSPCs. 4 weeks post transplantation the mice were treated with either isotype control antibody or ABTx135 (see Tables 26 and 27). 2 weeks (Dosing Paradigm A) or 24 hours (Dosing Paradigm B) after antibody dosing, a second transplant was performed with 1E+6 cells multiplex edited using the base editor ABE8.8 in combination with sgRNA CC128 (i.e., gRNA434; “CD117 edit cells”) and sgRNA_015 (“HBG1/2 edit cells”). Mice were sacrificed at the time points indicated in Tables 26 and 27, and bone marrow was harvested and one or more of the following was evaluated: A) base editing in the cells, as determined using next-generation sequencing (NGS); B) chimerism, C) immune cell immunophenotypes; D) receptor occupancy (RO); E) globin. Tables 26 and 27 provide details relating to mAb treatment and transplantation for the mouse groups. As controls, mice were administered an isotype antibody that did not bind wild type CD117 and/or the mice were administered cells that were only base edited using the base editor ABE8.8 in combination with only the guide sgRNA_015 (“HBG1/2 edit cells”).

TABLE 26
Description of groups of mice treated according
“Dosing Paradigm A” (see FIG. 63).

| | Cells (1e6/mouse) | | | | |
|---|---|---|---|---|---|
| | Administered ~4 | | | | |
| | wks After Administration | | | | |
| | of 1e6 wild type | | Take Down | | |
| | (unedited) healthy | Antibody | (TD) | TD | TD |
| Group | human CD34+ HSPCs | Administered | (0 wk) | (2 wk) | (12 wk) |
| 1 | HBG1/2 edit | Isotype | N = 5 | N = 2 | N = 5 |
| 2 | HBG1/2 edit | ABTx135 | | N = 2 | N = 5 |
| 3 | HBG1/2 edit + CD117 edit | Isotype | | N = 3 | N = 5 |
| 4 | HBG1/2 edit + CD117 edit | ABTx135 | | N = 3 | N = 5 |

“HBG1/2 edit” indicates that the cells were edited using the base editor ABE8.8 in combination with sgRNA_015, and “CD117 edit” indicates that the cells were edited using the base editor ABE8.8 in combination with sgRNA CC128 (gRNA434).

TABLE 27
Description of groups of mice treated according to
“Dosing Paradigm B” (see FIG. 63).

| | Cells (1e6/mouse) | | | | |
|---|---|---|---|---|---|
| | Administered ~4 | | | | |
| | wks After Administration | | | | |
| | of 1e6 wild type | | Take Down | | |
| | (unedited) healthy | Antibody | (TD) | TD | TD |
| Group | human CD34+ HSPCs | Administered | (0 wk) | (10 wk) | (12 wk) |
| 1 | HBG1/2 edit | Isotype | N = 0 | N = 5 | N = 5 |
| 2 | HBG1/2 edit | ABTx135 | | N = 5 | N = 5 |
| 3 | HBG1/2 edit + CD117 edit | Isotype | | N = 5 | N = 5 |
| 4 | HBG1/2 edit + CD117 edit | ABTx135 | | N = 5 | N = 5 |

“HBG1/2 edit” indicates that the cells were edited using the base editor ABE8.8 in combination with sgRNA_015, and “CD117 edit” indicates that the cells were edited using the base editor ABE8.8 in combination with sgRNA CC128 (gRNA434).

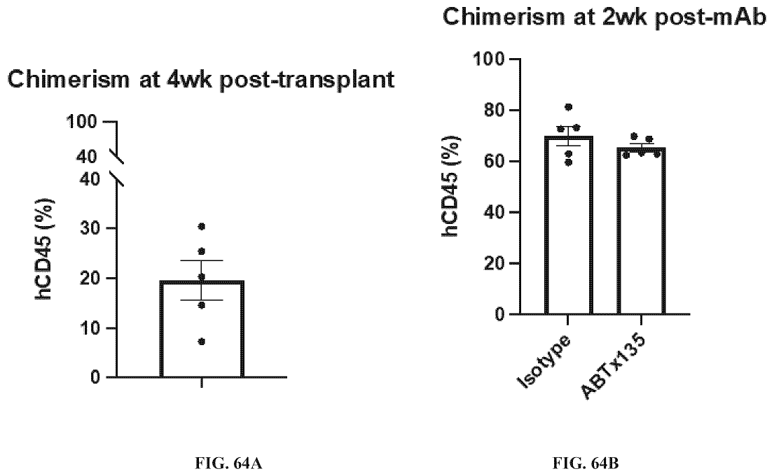

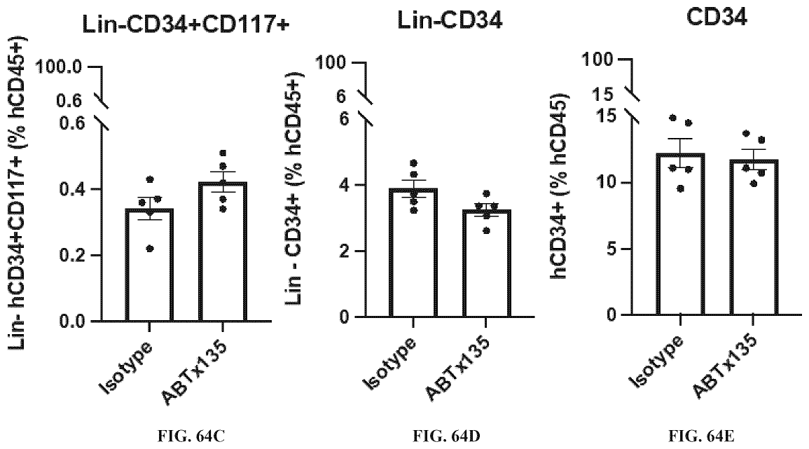

Analysis of immune cells in mice treated according to Dosing Paradigm A at two weeks following administration of the monoclonal antibody confirmed that administration of the antibody led to reductions in myeloid (CD15+) and erythrocyte (GlyA) cells in the mice (FIGS. 64A-64G). This confirmed that the antibody can be used as a way to deplete a bone marrow niche to facilitate engraftment of the cleared niche by a hematopoietic stem cell transplant.

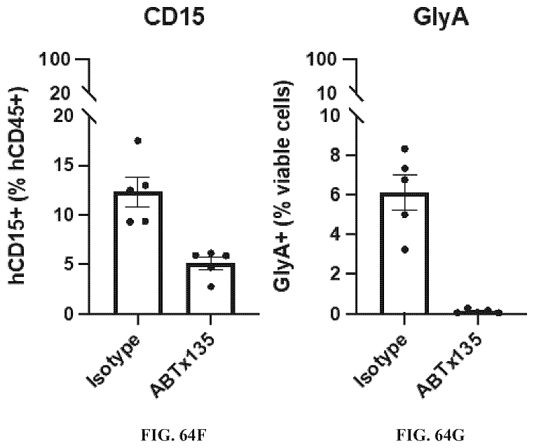

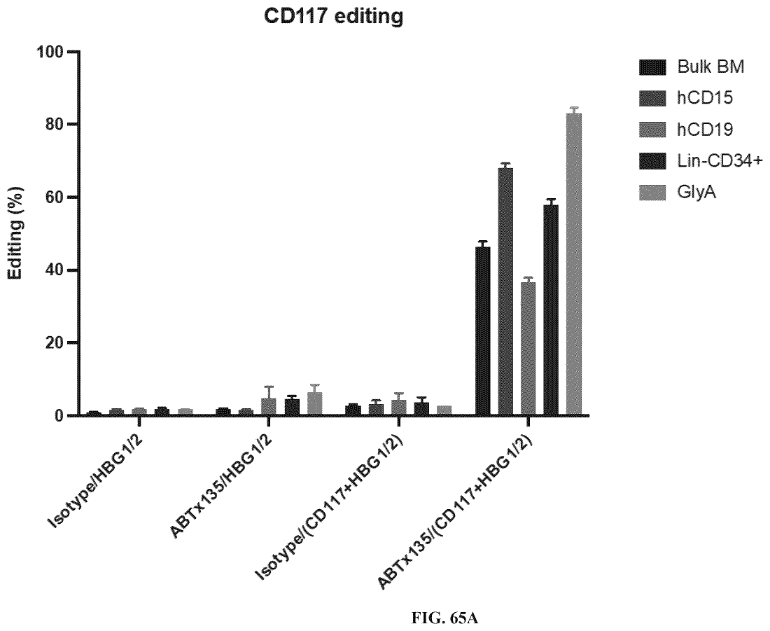

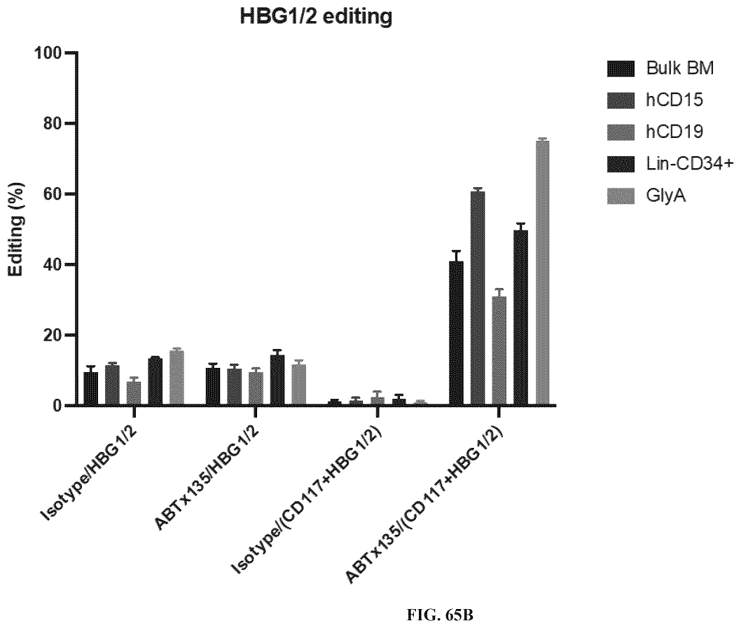

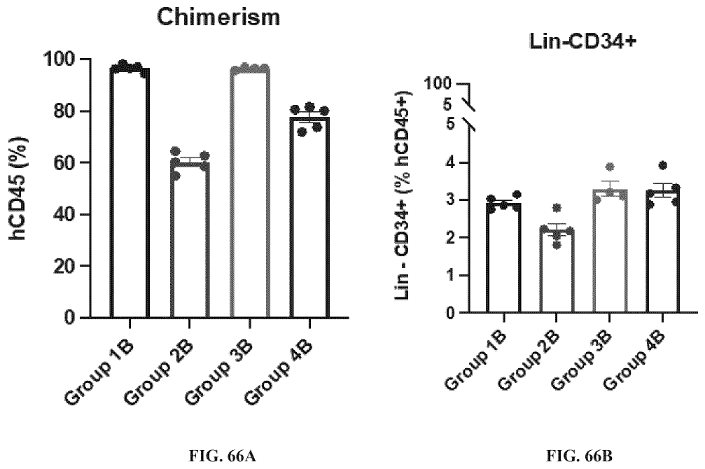

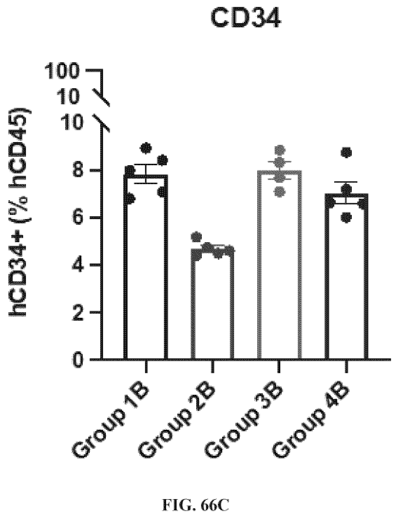

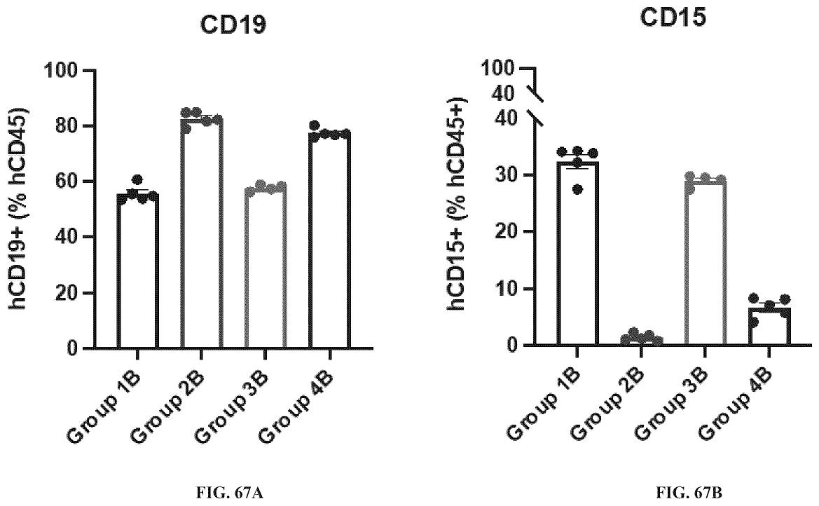

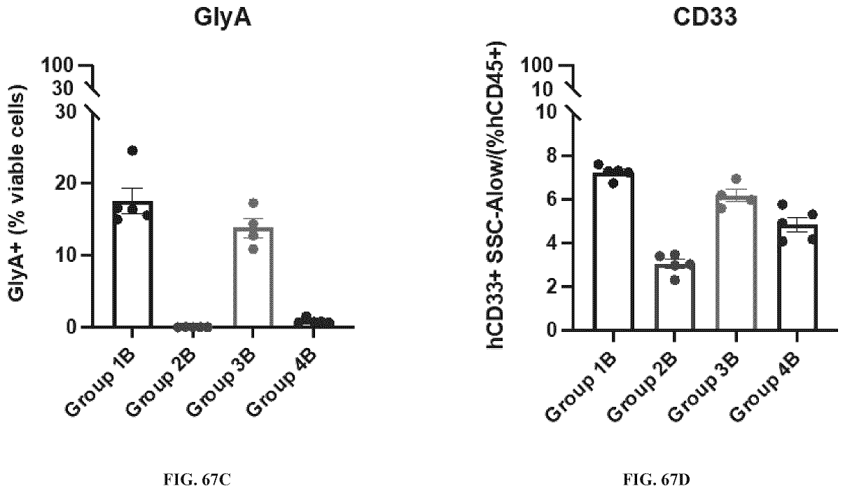

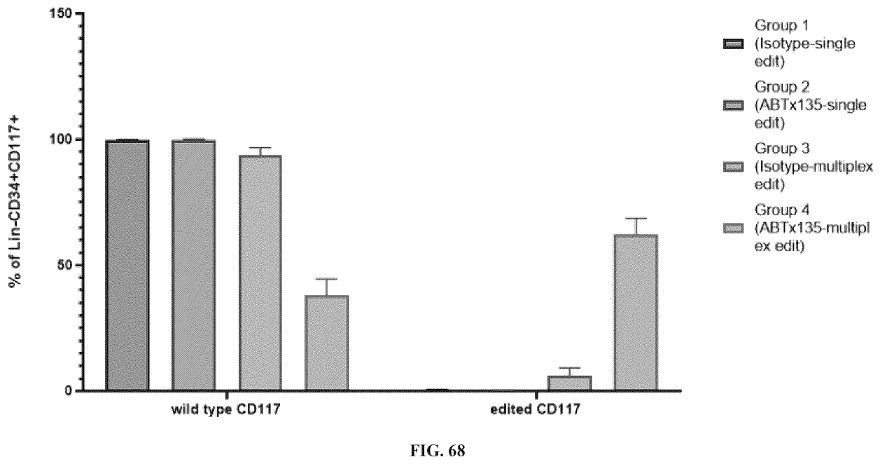

At 10 weeks post administration of base edited CD34+HSPCS to mice treated according to Dosing Paradigm B, a part of the bulk bone marrow from each mouse group was characterized. First, human CD15+, Human CD19+, and Lin-human CD34+, and GlyA+ cells were isolated from the bone marrow using flow assisted sorting at 10 weeks following administration of the monoclonal antibodies. Genomic DNA was isolated from the sorted cells and was subjected to next generation sequencing. FIGS. 65A and 65B show levels of on-target editing in the sorted cell populations within the CD117 (FIG. 65A) and HBG1/2 (FIG. 65B) sites targeted for base editing. Levels of target base editing was highest in cells collected from mice administered the “HBG1/2 edit+CD117 edit” cells post monoclonal antibody treatment using ABTx135. The mouse bone marrow was also interrogated using flow cytometry to confirm cell engraftment and multi-lineage reconstitution. FIGS. 66A-66C show human CD45+ chimerism (FIG. 66A), human Lin−CD34+ engraftment (FIG. 66B), and CD34+ engraftment (FIG. 66C) within mouse bone marrow. Frequencies of HSPCs and hematopoietic stem cells were comparable between isotype antibody treated mice administered cells with the HBG1/2 edit or with cells containing both the HBG1/2 edit and the CD117 edit. Administration of ABTx134 resulted in lower frequencies of CD34+ and Lin(−)CD34+ cells in mice that received only HBG1/2 edit cells (Group 2 in Table 27). Frequencies of CD34+ and Lin(−)CD34+ cells were higher in ABTx135-treated mice that were transplanted with HBG1/2 edit+CD117 edit cells (Group 4 in Table 27). Frequencies of CD19+, CD15+, GlyA+, and CD33+ cell populations were comparable between isotype antibody-treated groups engrafted with HBG1/2 edit cells or HBG1/2 edit+CD117 edit cells (FIGS. 67A-67D, Groups 1B and 3B). Administration of the ABTx135 monoclonal antibody (Groups 2 and 4) to the mice resulted in lower frequencies of GlyA+, CD33+, and CD15+ cells as compared to mice administered only the isotype antibody, and CD15+ and CD33+ cells were observed at higher frequencies in mice transplanted with HBG1/2 edit+CD117 edit cells. Levels of cells surface expressing wild type or edited CD117 expressed by cells containing the CD117 edit was also determined using flow cytometry. The cells were contacted ex vivo with a saturating dose of ABTx135, detected by anti-IgG-PE, which allowed for discrimination between wild type CD117 cells capable of binding ABTx135/1gG-PE and CD117 edit cells, which escaped ABTx135 binding. Mice administered HBG1/2 edit+CD117 edit cells contained the highest levels of cells surface-expressing edited CD117 polypeptides (i.e., CD117 polypeptides expressed by CD117 edit cells) (FIG. 68).

It was determined that both Dosing Paradigm A and Dosing Paradigm B yielded similar results in terms of, for example, engraftment and multi-lineage reconstitution.

The above results demonstrate that a single dose of an anti-CD117 monoclonal antibody can be used as a successful strategy for clearing a bone marrow niche to allow for engraftment in a subject of cells base edited to express a CD117 polypeptide that is not bound by or that only weakly binds the anti-CD117 monoclonal antibody.

### Example 10: Transplant of Base Edited Hematopoietic Stem Cells (HSCs) into NSG-SGM3 Mice Expressing Human Stem Cell Factor

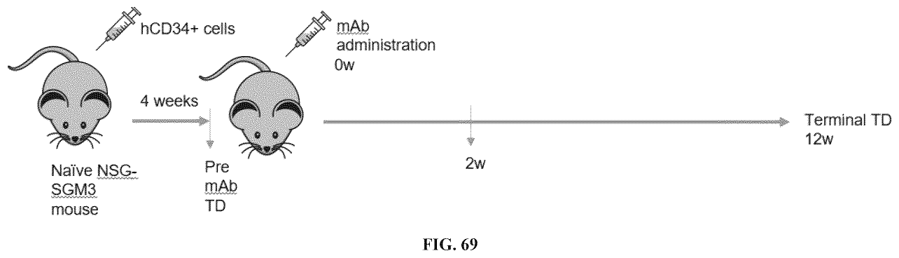

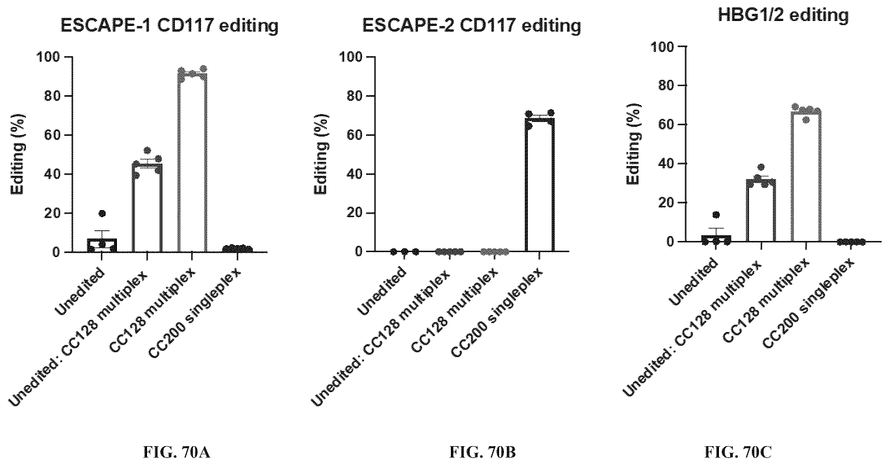

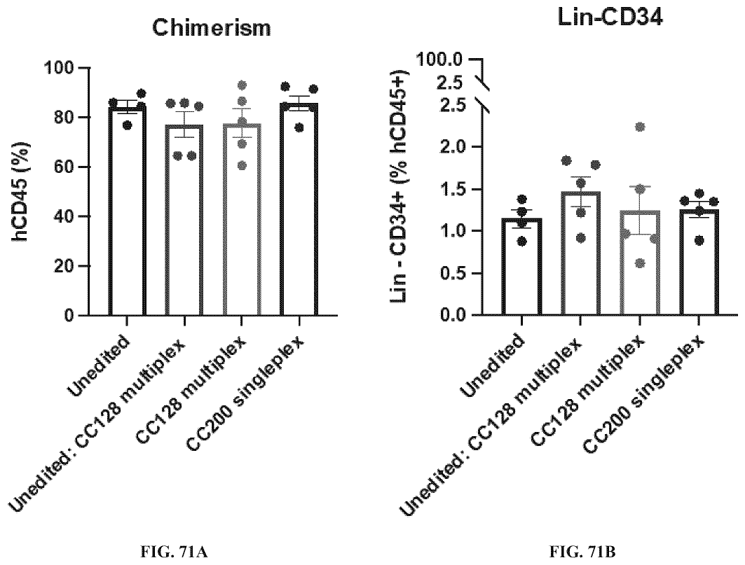

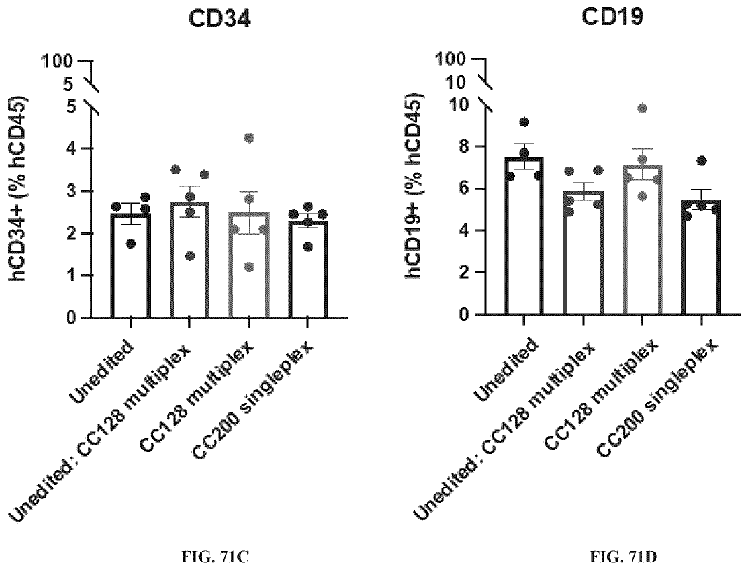

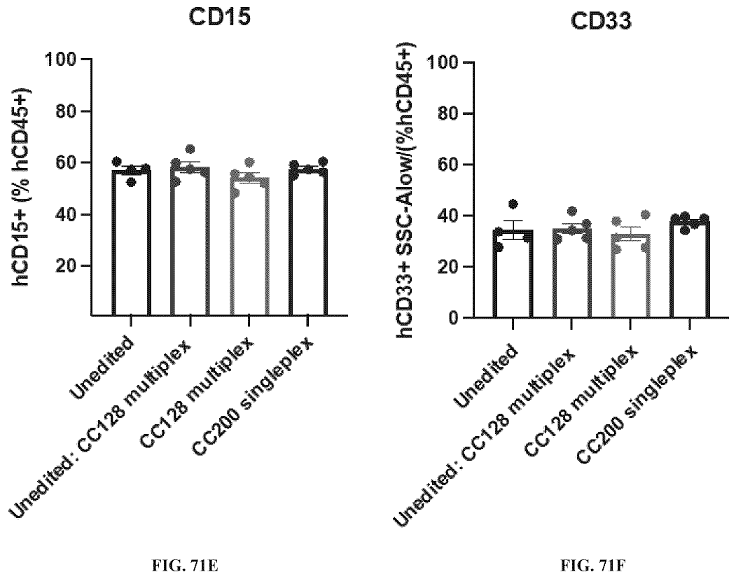

Since the above in vivo engraftment experiments used mice expressing a mouse stem cell factor (SCF), experiments were undertaken to demonstrate improved engraftment of base edited hematopoietic stem cells (HSCs) in mice expressing a human stem cell factor. The mice were not previously humanized (i.e., transplanted with 1e6 wild type (unedited) healthy human CD34+HSPCs). As shown in FIG. 69, NSG-SGM3 mice, which express a human stem cell factor, were treated according to the protocol described in FIG. 69 and in Table 28 below. Endpoint readouts included the following: immunophenotyping using flow cytometry and a combined antibody panel (FIGS. 71A-71F); and next-generation sequencing to characterize base editing detected in bulk bone marrow (BM) (FIGS. 70A-70C). It was found that the cells administered to the mice demonstrated high levels of engraftment as well as multilineage reconstitution at 4 weeks following administration of the human CD34+ cell transplants. Engraftment and frequencies of measured cell populations were comparable between edited and unedited cells (FIGS. 71A-71F). The results also demonstrated higher levels of engraftment in the mice expressing the human stem cell factor (hSCF) as compared to mice expressing only the mouse stem cell factor (mSCF), thereby showing that levels of engraftment of cells expressing an edited CD117 polypeptide previously observed in mice were lower than would have been the case if the mice expressed a human SCF (compare, for example, FIG. 66A, Group 4 with FIG. 70A “CC128 Multiplex.”

TABLE 69
Description of groups of mice treated (see FIG. 69).

| | | | mAb | | | |
|---|---|---|---|---|---|---|
| | Cells Administered (1e6 | | Dose | N | N | N |
| Group | cells/mouse) | mAb | (mg/kg) | (0 w) | (2 w) | (12 w) |
| 1 | Unedited | No mAb | 0 | 5 | 5 | 5 |
| 2 | Unedited:HBG1/2 edit + CC128 | Isotype | 20 | 5 | 5 | 5 |
| | edit (CC128 Multiplex) | | | | | |
| 3 | HBG1/2 edit + CC128 edit | No mAb | 0 | 5 | 5 | 5 |
| | (CC128 Multiplex) | | | | | |
| 4 | CC200 edit (CC200 singleplex) | No mAb | 0 | 5 | 5 | 5 |

“HBG1/2 edit” indicates that the cells were edited using the base editor ABE8.8 in combination with sgRNA_015, “CC128 edit” indicates that the cells were edited using the base editor ABE8.8 in combination with sgRNA CC128 (gRNA434), and “CC200” indicates that the cells were edited using the base editor ABE8.20-NRCH, ABE9v1, or ABE9v2 in combination with sgRNA CC200 (gRNA931). The term “Unedited:HBG1/2 edit + CC128 edit (CC128 Multiplex)” indicates that the mice were administered a 1-to-1 mix of unedited cells and multiplex base edited cells (i.e., HBG1/2 edit + CC128 edit cells).

### Example 11: Affinity Maturation of Anti-CD117 Antibodies

Experiments were undertaken to use a yeast display platform available from the company “Curia” for the affinity maturation of the monoclonal antibody ABTx025. ABTx025 had specific binding for wild type CD117, was cross-reactive with cyno CD117, and blocked binding of stem cell factor (SCF) to CD117. The objective of the experiments was to identify affinity matured antibodies with improved affinity for wild type human CD117 (hu-cKIT) and low-to-no binding to CC128 CD117 (cKIT) variants. The CC128 CD117 variants corresponded to those CD117 polypeptides expressed from variants of polynucleotides encoding wild type CD117 prepared through base edited thereof using the base editor ABE8.8 in combination with the guide polynucleotide sgRNA_015. The affinity maturation involved generating random mutants of ABTx025 followed by negative selection and counter screening against the CC128 CD117 variant. Eleven (11) unique variants of ABTx025 were identified using the yeast display platform.

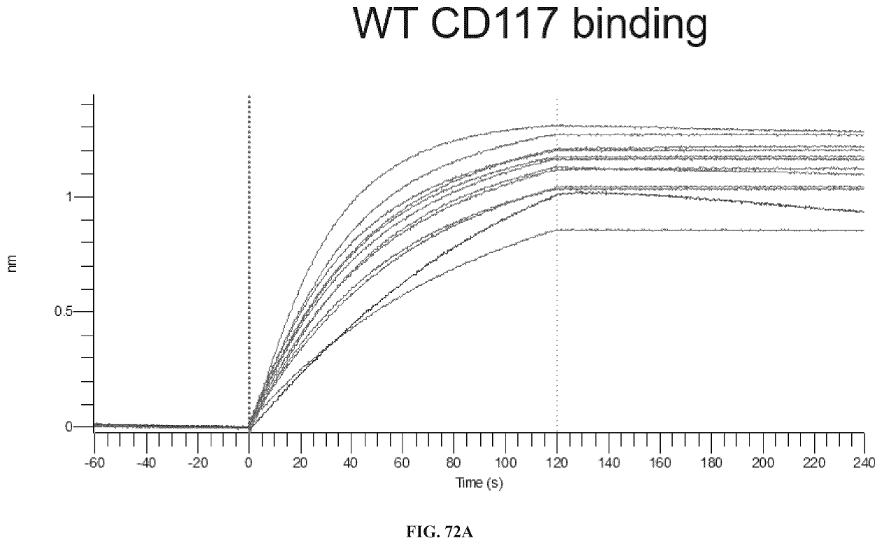

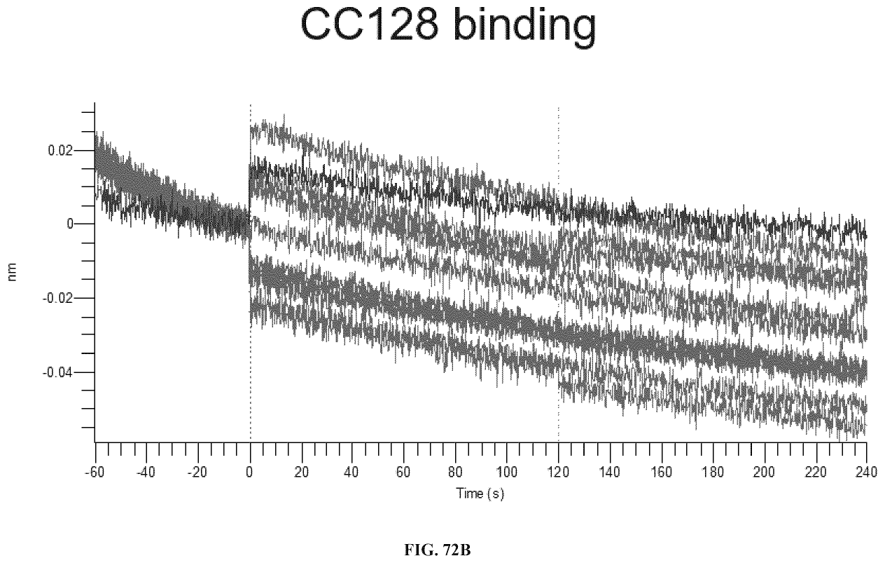

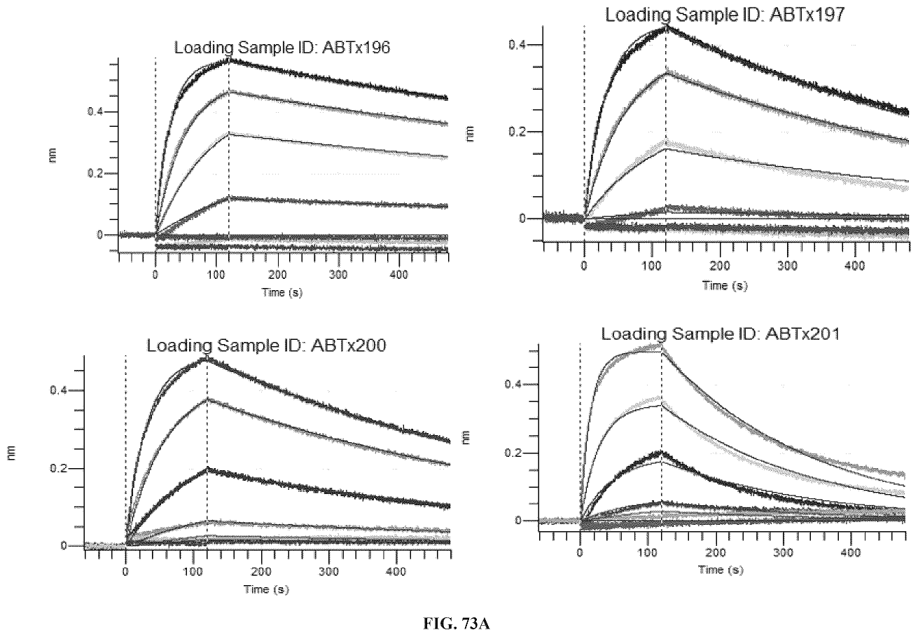

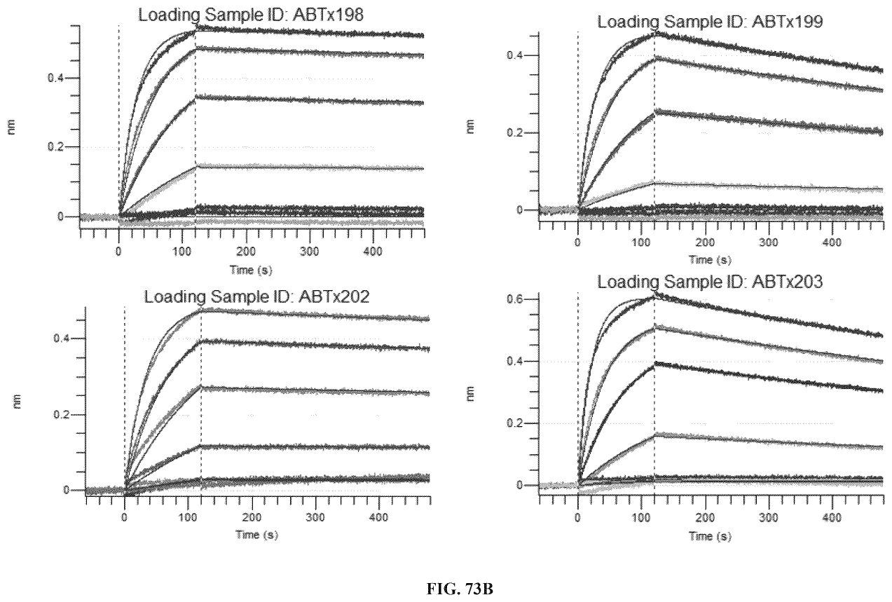

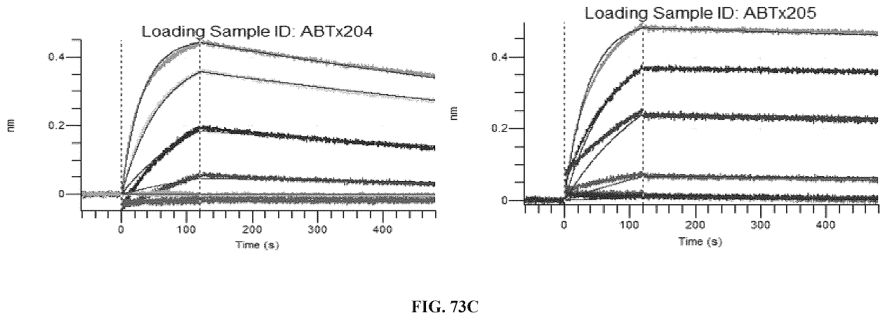

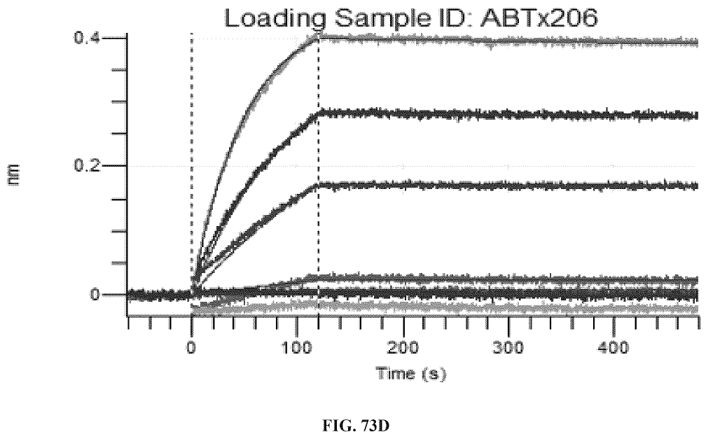

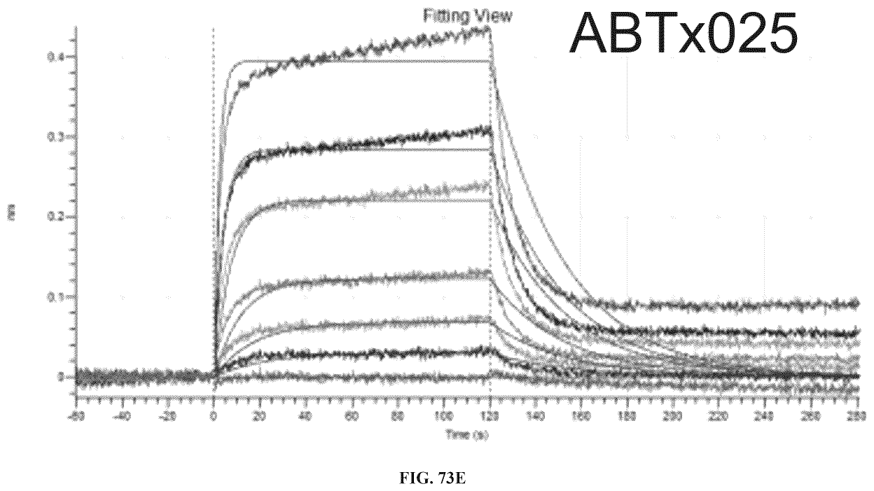

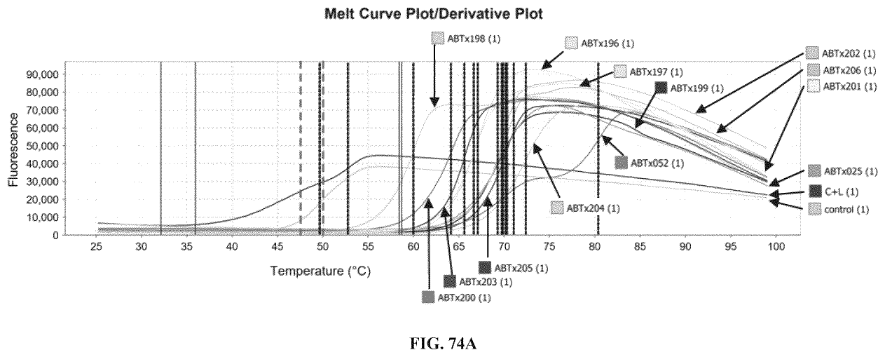

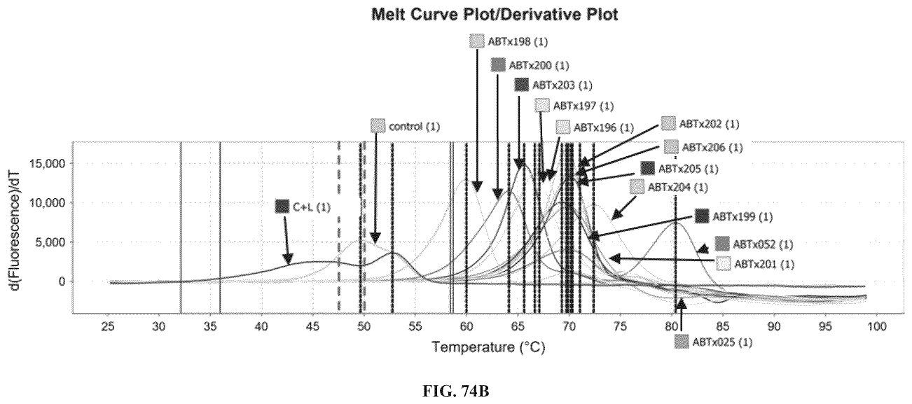

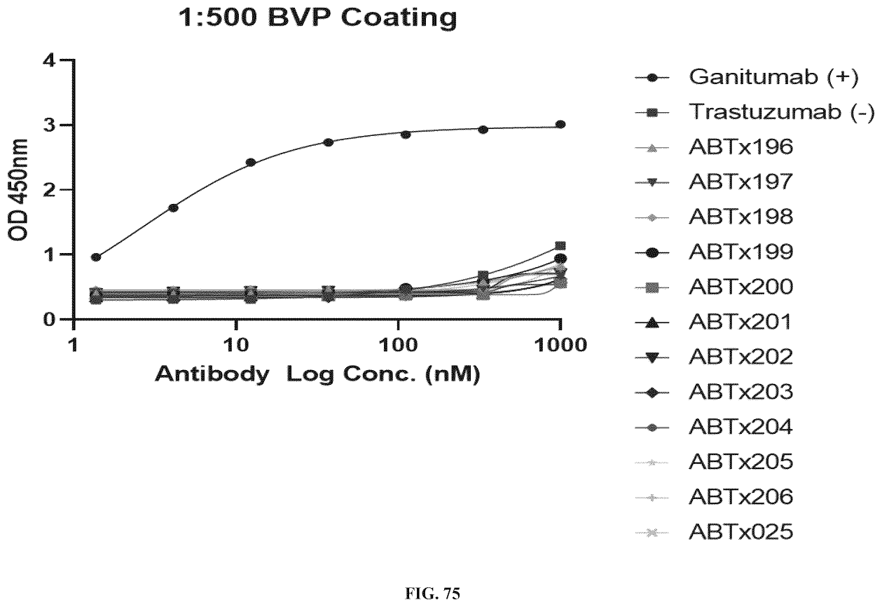

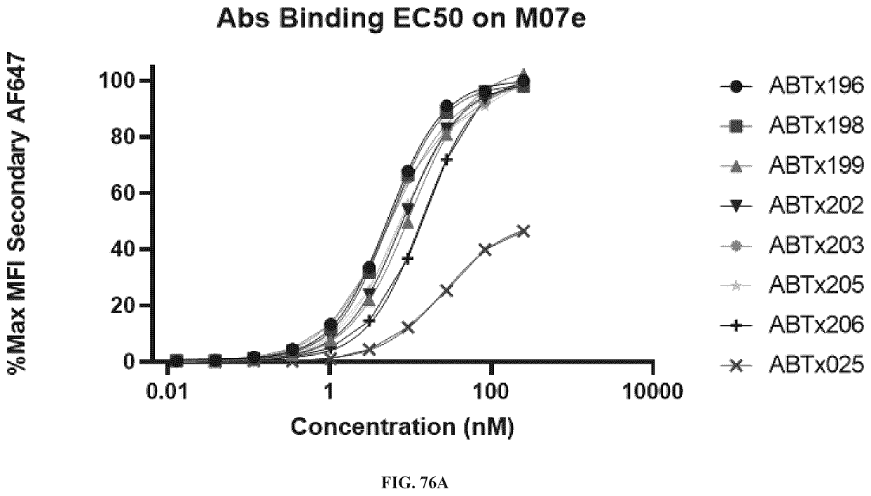

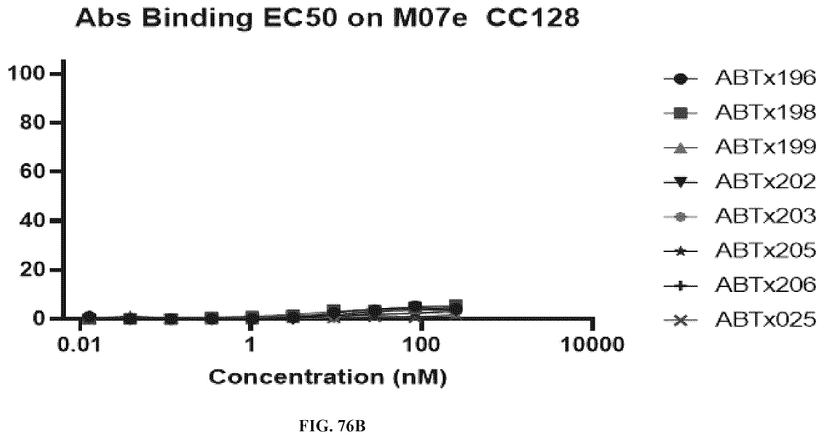

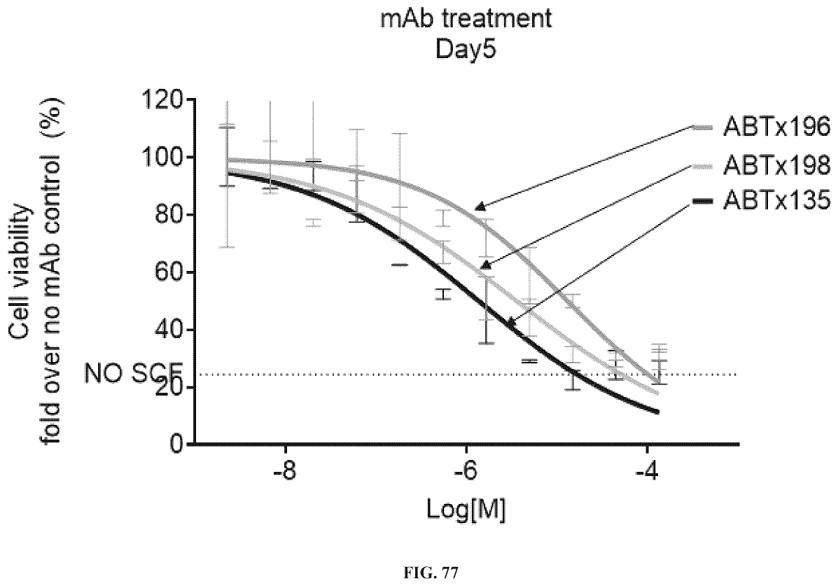

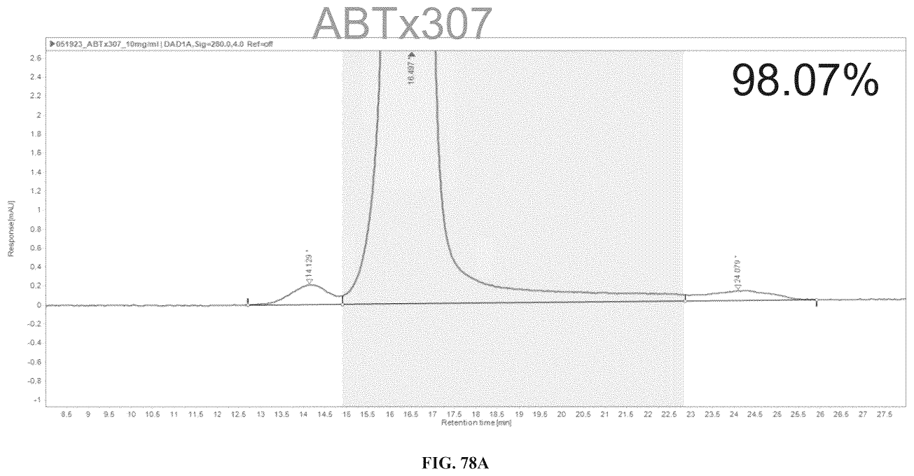

The 11 IgG1 variants of ABTx025 identified using the yeast display platform were expressed in Chinese hamster ovary (CHO) cells, purified (see Table 70), and characterized. The following properties of the antibodies were characterized: 1) wild type CD117 and CC128 CD117 variant binding including monovalent KD measurement (Table 71); Tm as determined by differential scanning fluorimetry (DSF) (FIGS. 74A and 74B and Table 72); polyspecificity; EC50 on M07e cells (FIGS. 76A and 76B and Table 73); and stem cell factor (SCF) blocking (FIG. 77). Most of the 11 variants showed improved binding affinity to wild type CD117 and showed no binding to the CC128 variant of CD117 (FIGS. 72A and 72B), and many of the variants showed slow off rates for binding (FIGS. 73A-73E). Seven of the eleven variants of ABTx025 had comparable monovalent KD values as compared to ABTx052 (Table 71). All of the 11 variants had low polyspecificity (FIG. 75). Most of the variants had high melting temperatures (FIGS. 74A and 74B, and Table 72). The variants ABTx196 and ABTx198 were able to efficiently deplete human CD34+ hematopoietic stem and progenitor cells (HSPCs) with efficiencies comparable to the monoclonal antibody ABTx135 (FIG. 77 and Table 74). Table 75 provides a summary of the characterization of the eleven (11) variants.

TABLE 70
Purity of antibodies obtained from CHO cells. The
antibodies were produced as human IgG1 format.

| | | Purity | Purity | | |
|---|---|---|---|---|---|
| | Concen- | determined | determined | | |
| Antibody | tration | by SDS- | by SEC- | Endotoxin | Total |
| ID | (mg/ml) | PAGE(%) | HPLC(%) | (EU/mg) | (mg) |
| ABTx196 | 1.65 | ≥95 | 97 | ≤0.1 | 2.8 |
| ABTx197 | 1.38 | ≥95 | 97 | ≤0.1 | 2.48 |
| ABTx198 | 1.22 | ≥80 | 93 | ≤0.1 | 2.19 |
| ABTx199 | 2.04 | ≥90 | 96 | ≤0.1 | 3.67 |
| ABTx200 | 2.03 | ≥95 | 94 | ≤0.1 | 3.65 |
| ABTx201 | 1.15 | ≥95 | 97 | ≤0.1 | 1.95 |
| ABTx202 | 1.71 | ≥95 | 98 | ≤0.1 | 3.07 |
| ABTx203 | 0.96 | ≥95 | 98 | ≤0.1 | 1.72 |
| ABTx204 | 1.07 | ≥95 | 97 | ≤0.1 | 1.92 |
| ABTx205 | 1.69 | ≥95 | 82 | ≤0.1 | 2.87 |
| ABTx206 | 1.83 | ≥75 | 89 | ≤0.1 | 3.29 |

TABLE 71
KD values.

| Antibody | KD | ka | kdis | Full | Full |
|---|---|---|---|---|---|
| ID | (M) | (1/Ms) | (1/s) | X\hat{ }2 | R\hat{ }2 |
| ABTx196 | 8.18E−09 | 8.44E+04 | 6.91E−04 | 6.1357 | 0.9912 |
| ABTx197 | 2.93E−08 | 5.94E+04 | 1.74E−03 | 4.011 | 0.9864 |
| ABTx198 | 1.20E−09 | 7.28E+04 | 8.74E−05 | 1.4725 | 0.9979 |
| ABTx199 | 8.70E−09 | 7.28E+04 | 6.33E−04 | 1.618 | 0.9964 |
| ABTx200 | 2.57E−08 | 6.36E+04 | 1.64E−03 | 0.5891 | 0.9982 |
| ABTx201 | 3.46E−08 | 1.27E+05 | 4.41E−03 | 2.3177 | 0.9911 |
| ABTx202 | 2.42E−09 | 5.78E+04 | 1.40E−04 | 0.9935 | 0.9978 |
| ABTx203 | 6.62E−09 | 9.71E+04 | 6.43E−04 | 1.0487 | 0.9985 |
| ABTx204 | 1.17E−08 | 8.44E+04 | 6.91E−04 | 2.3487 | 0.9941 |
| ABTx205 | 2.07E−09 | 5.66E+04 | 1.17E−04 | 2.1499 | 0.9956 |
| ABTx206 | 1.26E−09 | 4.03E+04 | 5.07E−05 | 1.3438 | 0.9962 |
| ABTx025 | 1.47E−06 | 3.72E+04 | 5.09E−02 | 1.2024 | 0.9169 |
| ABTx052 | 8.05E−09 | 3.07E+05 | 2.47E−03 | 4.7577 | 0.992 |

TABLE 72
Melting temperatures (Tms). The “control” sample
was provided with a kit used to measure melting temperatures,
and “C + L” indicates “control plus ligand.”

| Antibody ID | Tm1 | Tm2 |
|---|---|---|
| ABTx025 | 69.88 | |
| ABTx196 | 67.08 | |
| ABTx197 | 66.63 | |
| ABTx198 | 59.99 | 70.33 |
| ABTx199 | 69.29 | |
| ABTx200 | 64.12 | |
| ABTx201 | 71.06 | |
| ABTx202 | 69.74 | |
| ABTx203 | 65.6 | |
| ABTx204 | 72.39 | |
| ABTx205 | 70.18 | |
| ABTx206 | 70.18 | |
| ABTx052 | 69.88 | 80.37 |
| control | 49.65 | |
| C + L | 52.75 | |

TABLE 73
EC50 on M07e cells.

| Antibody ID | EC50 (nM) |
|---|---|
| ABTx196 | 5.215 |
| ABTx198 | 5.425 |
| ABTx199 | 9.978 |
| ABTx202 | 7.977 |
| ABTx203 | 5.56 |
| ABTx205 | 7.48 |
| ABTx206 | 14.15 |

TABLE 74
IC50 values.

| Antibody ID | IC50 (M) | R squared |
|---|---|---|
| ABTx196 | 1.30E−05 | 0.7431 |
| ABTx198 | 3.59E−06 | 0.8425 |
| ABTx135 | 1.36E−06 | 0.8672 |

TABLE 75
Summary of characterization of variants
of the monoclonal antibody ABTx025.

| | KD | Tm | | |
|---|---|---|---|---|
| Ab ID | (M) | (° C.) | BVP | EC50(nM) |
| ABTx196 | 8.18E−09 | 69.88 | Low | 5.215 |
| ABTx197 | 2.93E−08 | 67.08 | Low | 5.425 |
| ABTx198 | 1.20E−09 | 66.63 | Low | 5.425 |
| ABTx199 | 8.70E−09 | 59.99 | Low | 9.978 |
| ABTx200 | 2.57E−08 | 69.29 | Low | |
| ABTx201 | 3.46E−08 | 64.12 | Low | |
| ABTx202 | 2.42E−09 | 71.06 | Low | 7.977 |
| ABTx203 | 6.62E−09 | 69.74 | Low | 5.56 |
| ABTx204 | 1.17E−08 | 65.6 | Low | |
| ABTx205 | 2.07E−09 | 72.39 | Low | 7.48 |
| ABTx206 | 1.26E−09 | 70.18 | Low | 14.15 |
| ABTx025 | 1.47E−06 | 70.18 | Low | NS |
| ABTx052 | 8.05E−09 | 69.88 | Low | 0.17 |

### Example 12: Anti-CD117 Antibodies with Modified Fc Domains

IgG1 antibodies targeting CD117 can cause mast cell degranulation, which can result from Fcγ receptor interactions. Accordingly, it can be advantageous to modify the Fc domain of anti-CD117 antibodies include one or more amino acid residue alterations in an Fc domain to reduce or eliminate binding to FcγR, binding to FcRn, and/or mast cell activation. Therefore, variants of the antibodies ABTx307, ABTx308, ABTx309, were prepared to have Fc domains containing the alterations L234A, L235A, and D265A (LALADA); where the amino acid positions are referenced to the following amino acid sequence: SGAAVKKPGESLKISCKGSGYRFTSYWIGWVRQMPGKGLEWMGIIYPGDSDTRYSPSFQGQVTI SAGKSISTAYLOWSSLKASDTAMYYCARHGRGYNGYEGAFDIWGQGTMVTVSSASTKGPSVFPL APSSKSTSGGTAALGCLVKDYFPEPVTVSWNSGALTSGVHTFPAVLOSSGLYSLSSVVTVPSSS LGTQTYICNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFYPSDIAVEWESN GQPENNYKTTPPVLDSDGSFFLYSKLTVDKSRWQQGNVFSCSVMHEALHNHYTQKSLSLSPG (positions 7-1101 of SEQ ID NO: 419). Further, the antibody ABTx313 was prepared, which contained the amino acid alterations N104D and E107D in the heavy chain (i.e., in CDR3) and the alterations N92S in the light chain (i.e., in CDR3), where the alterations are relative to the heavy chain and light chains of the antibody ABTx052 (SEQ ID NOs: 419 and 420, respectively), together with an Fc domain containing the LALADA alteration. The antibodies were expressed in host cells, purified, and characterized.

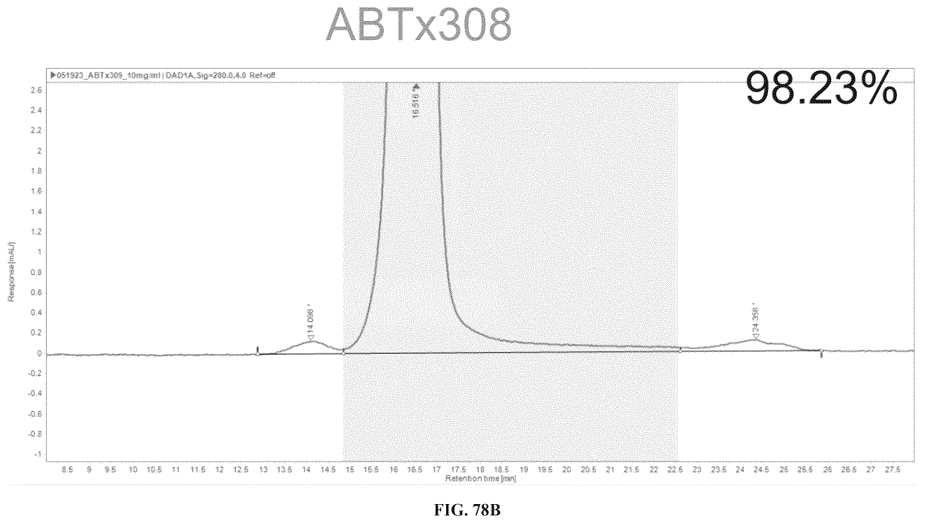

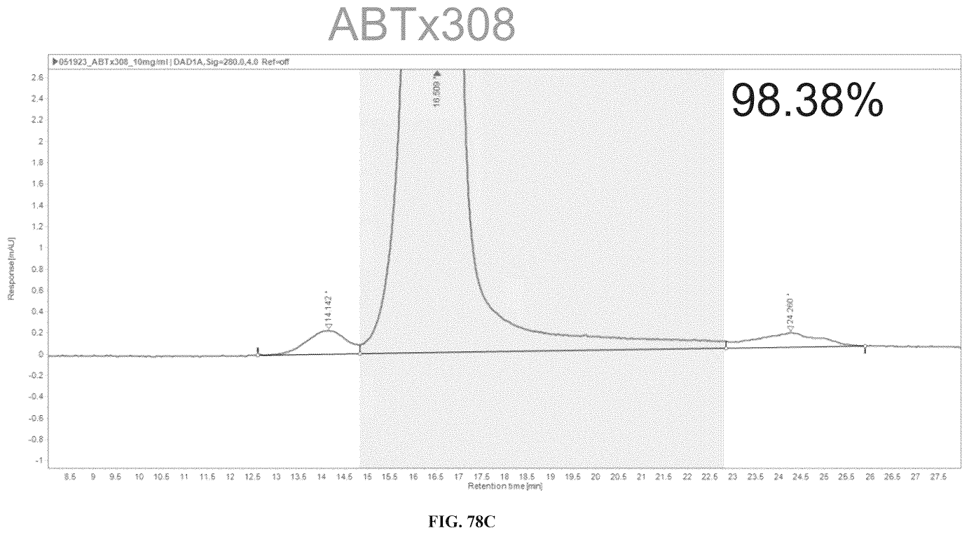

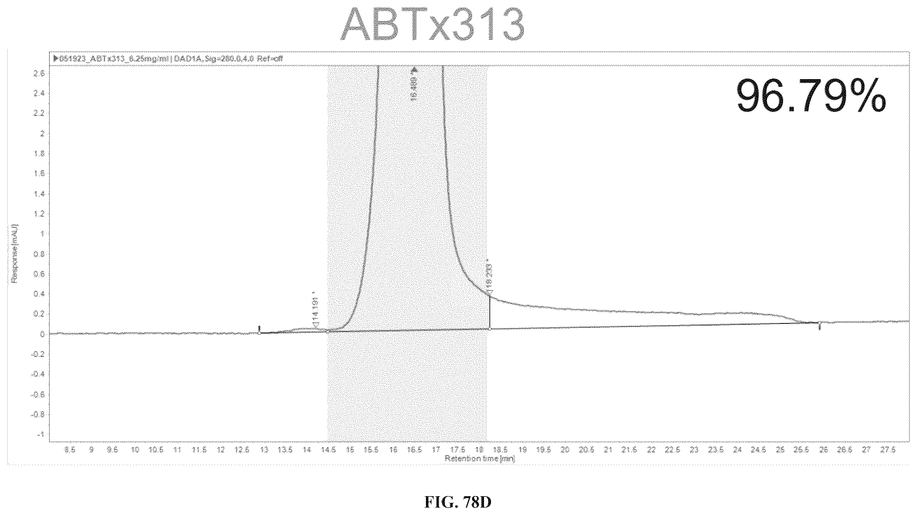

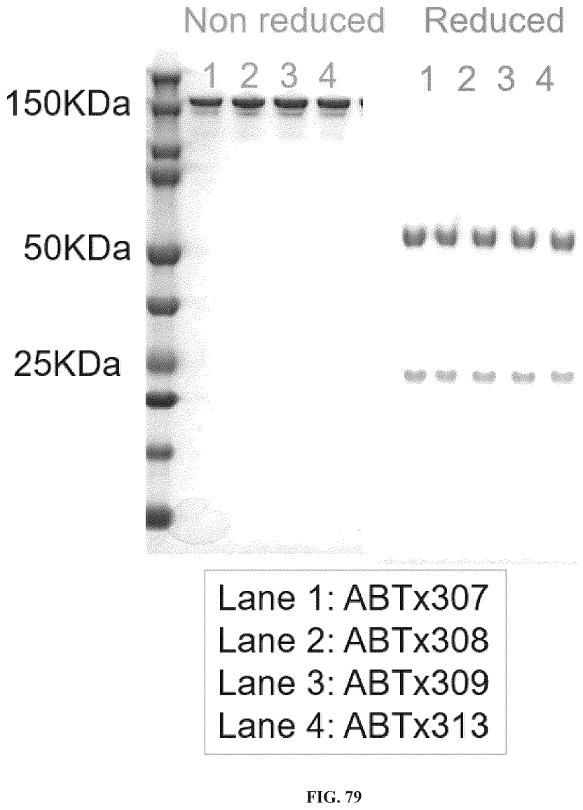

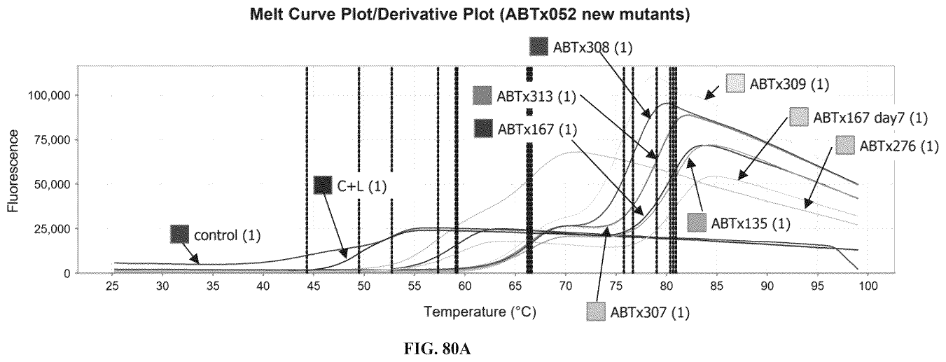

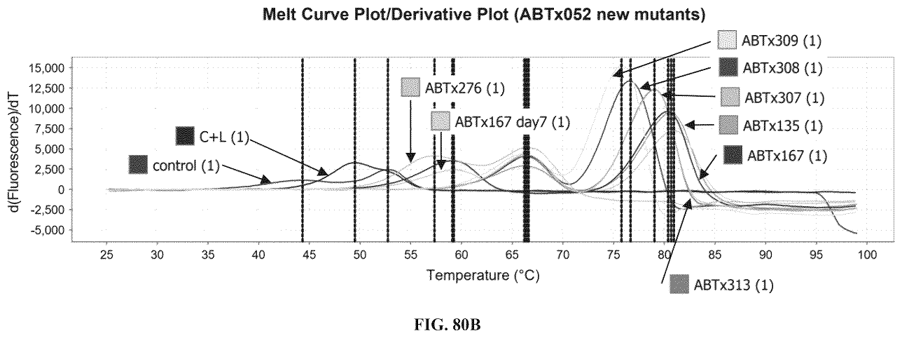

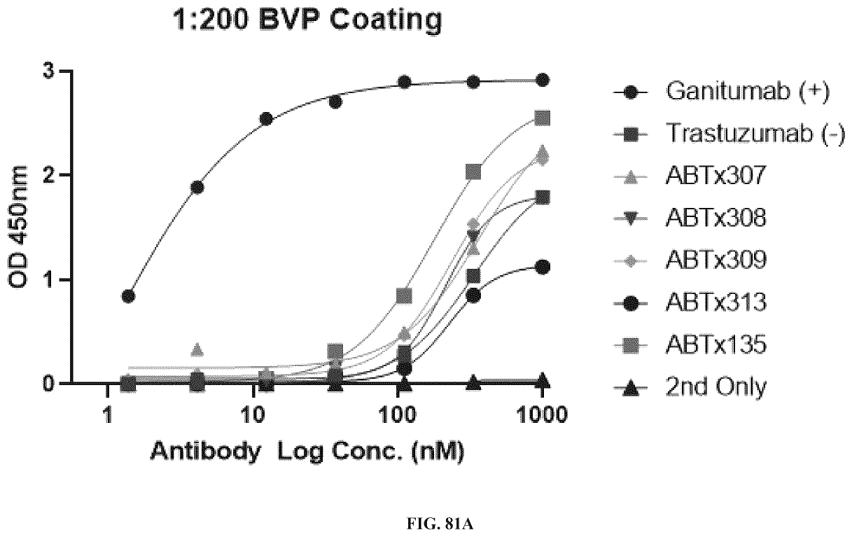

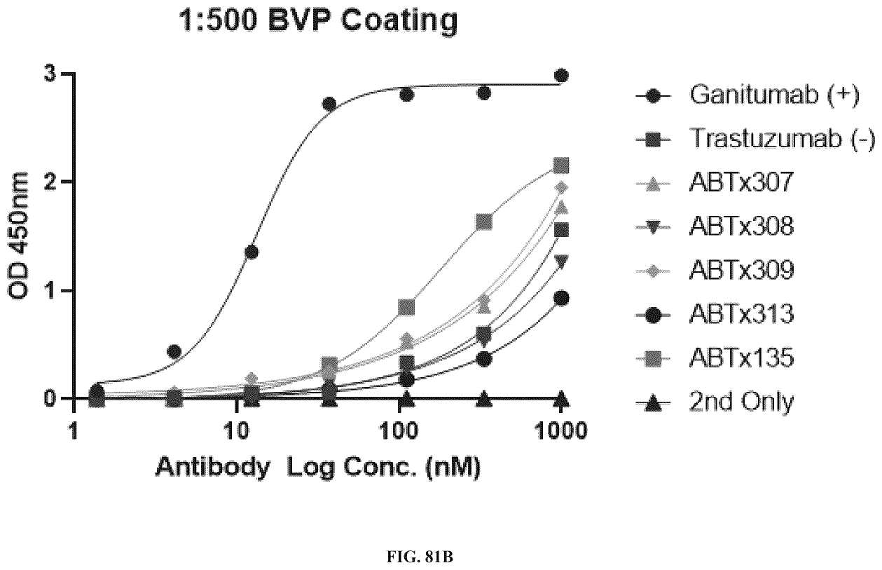

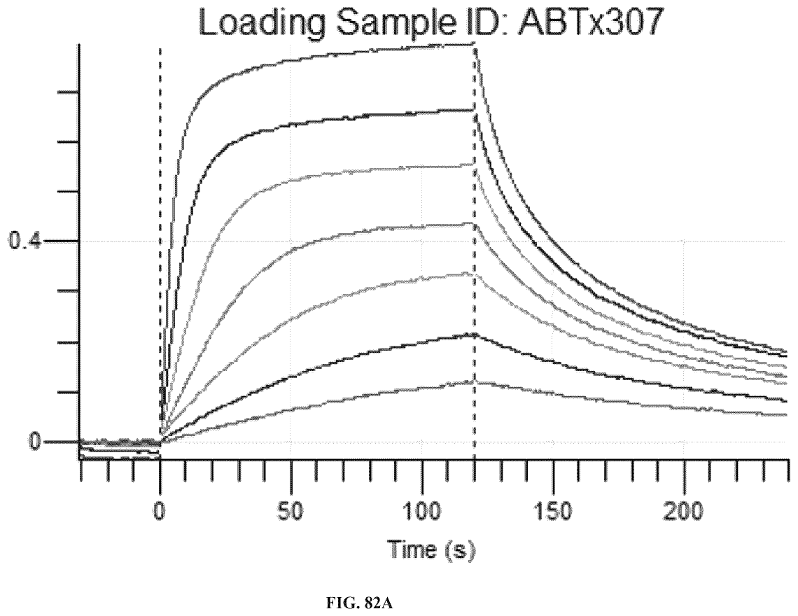

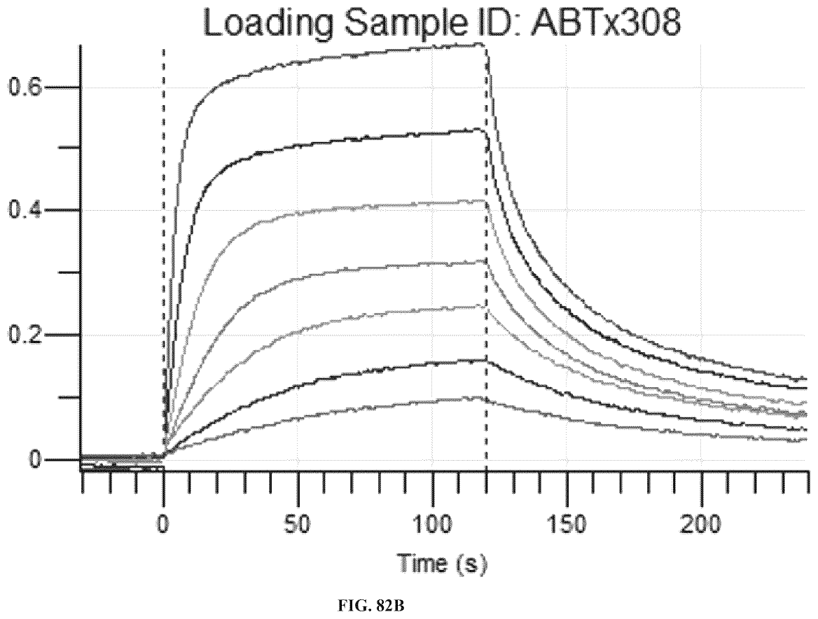

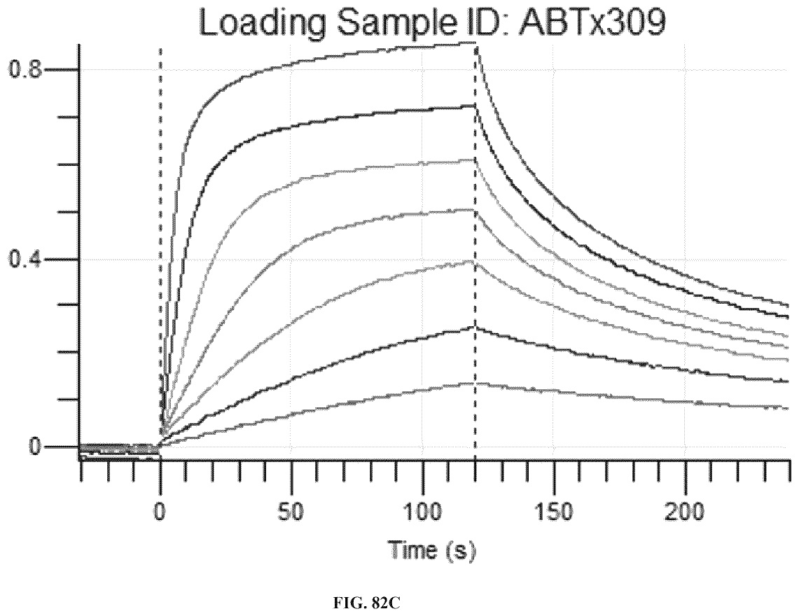

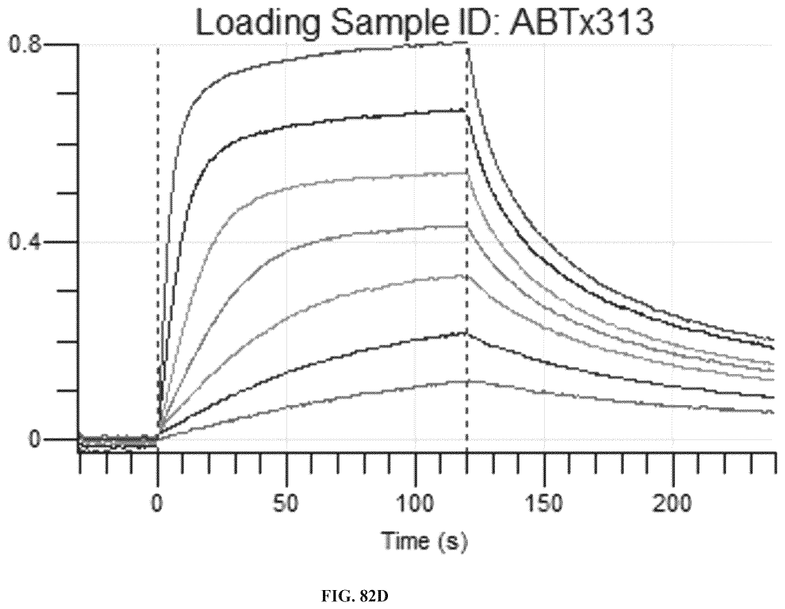

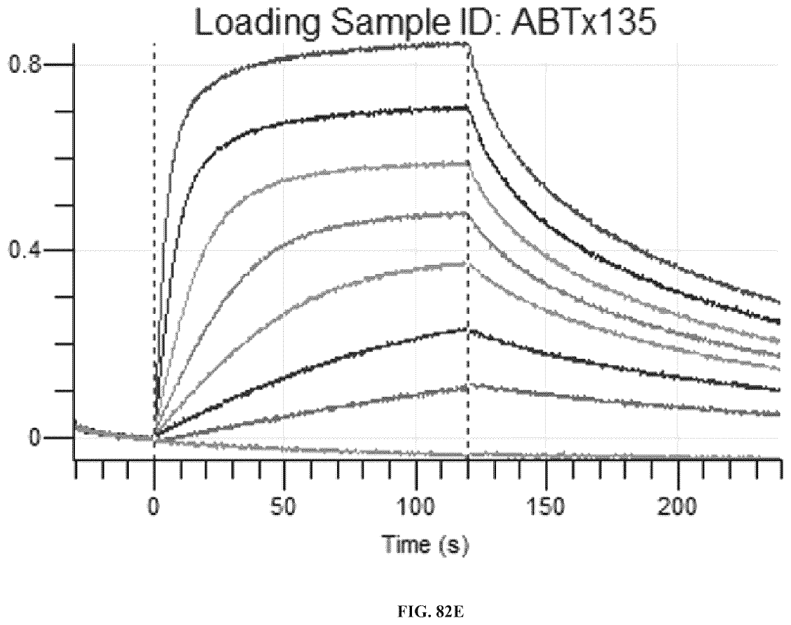

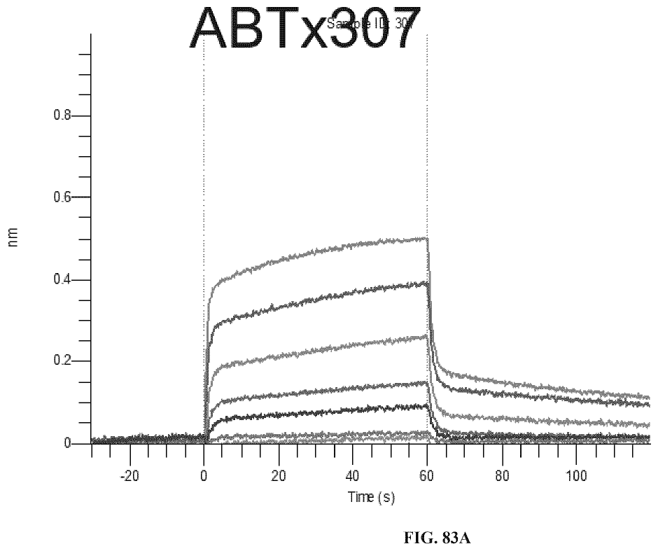

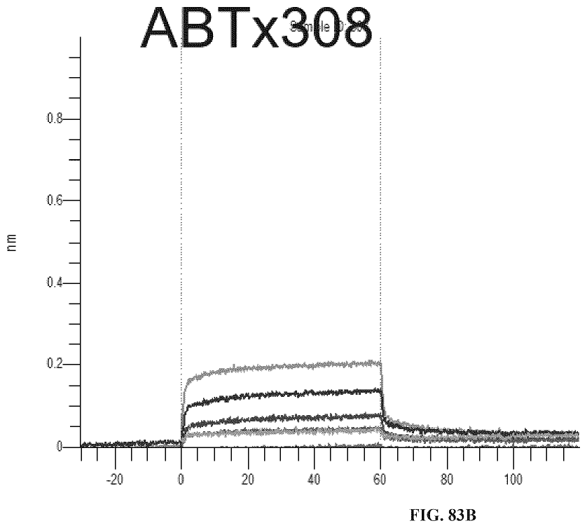

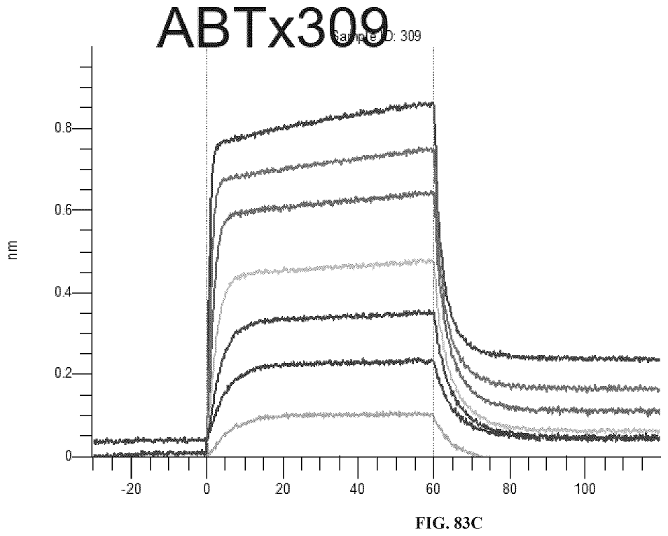

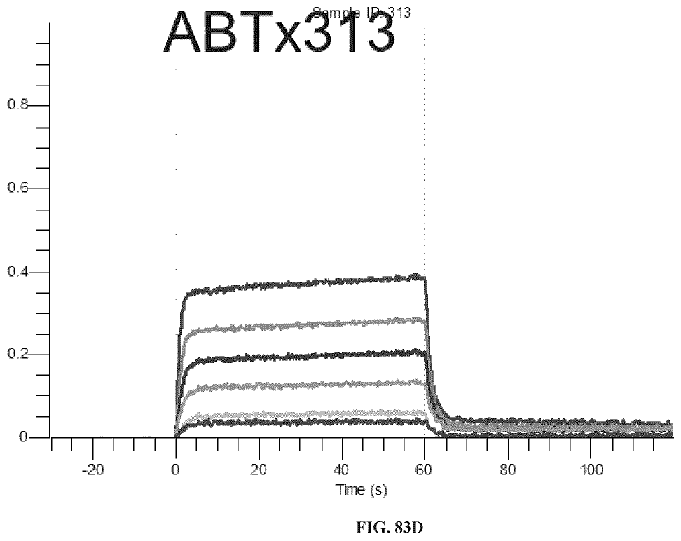

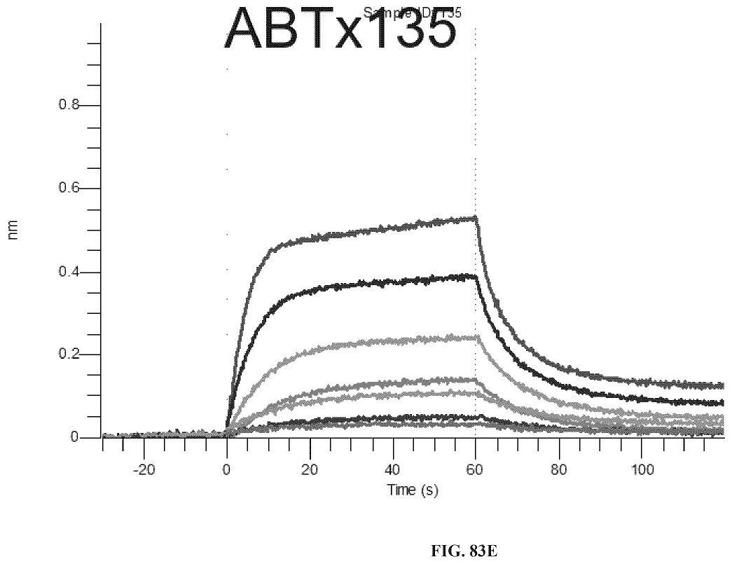

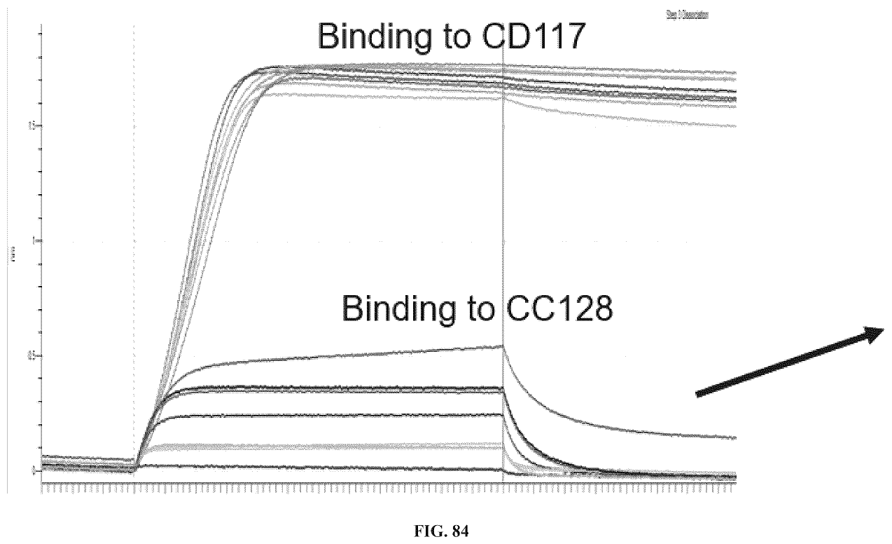

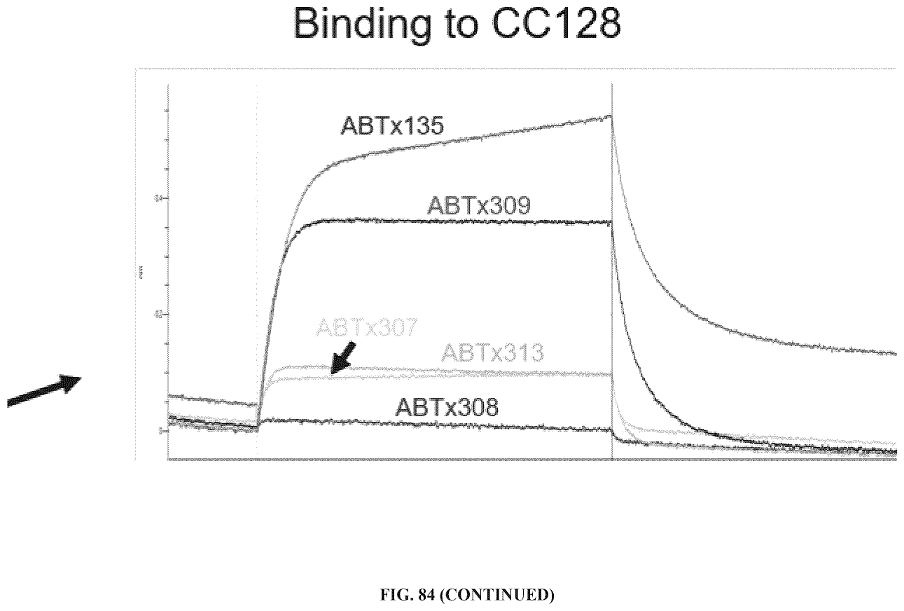

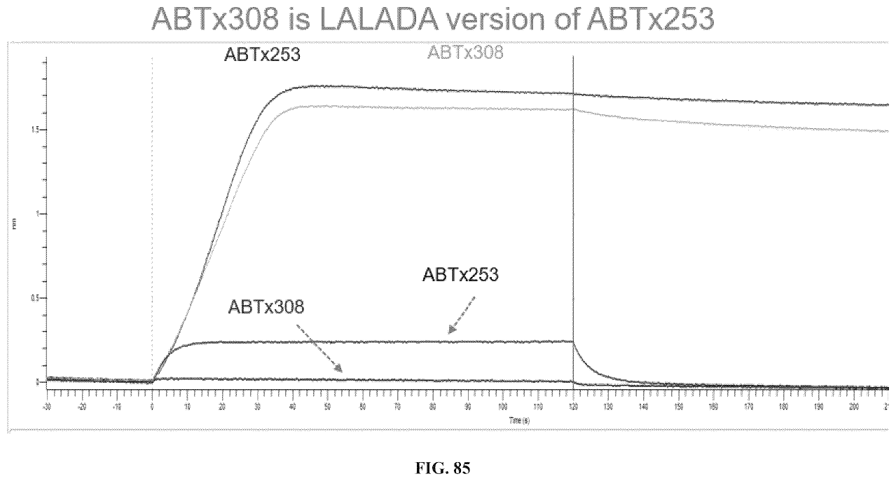

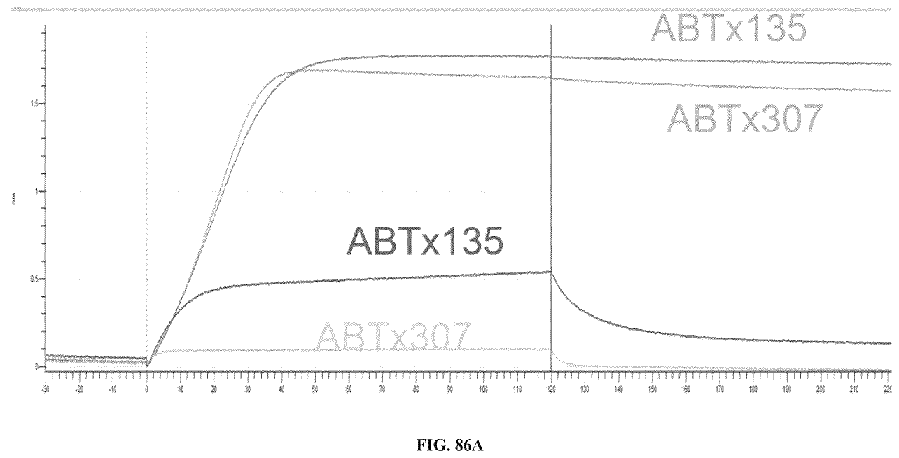

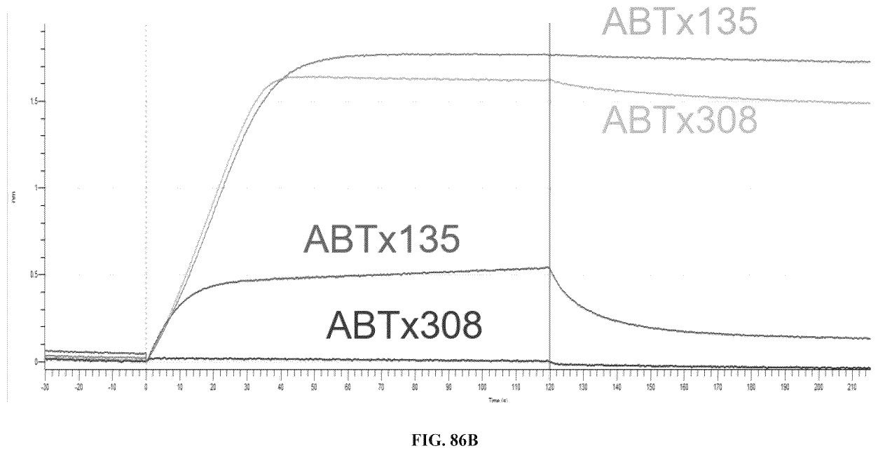

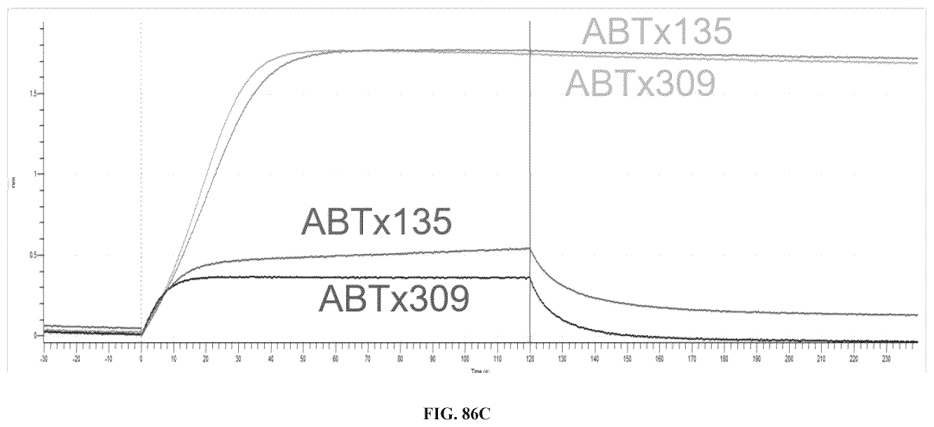

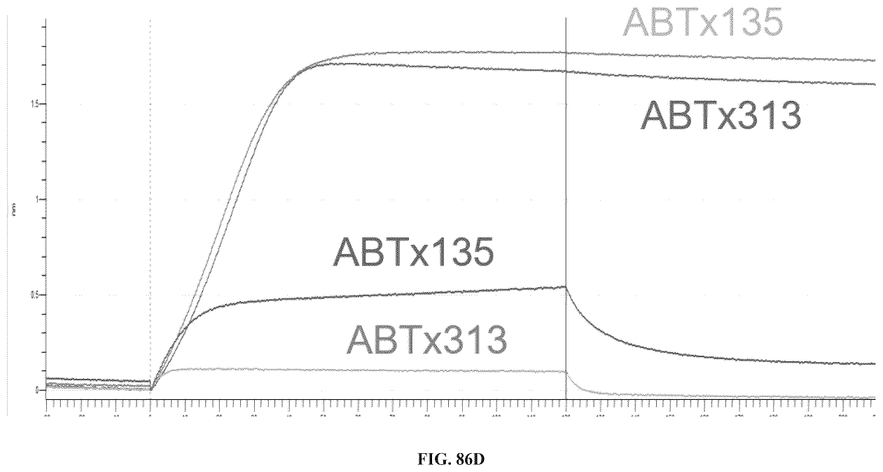

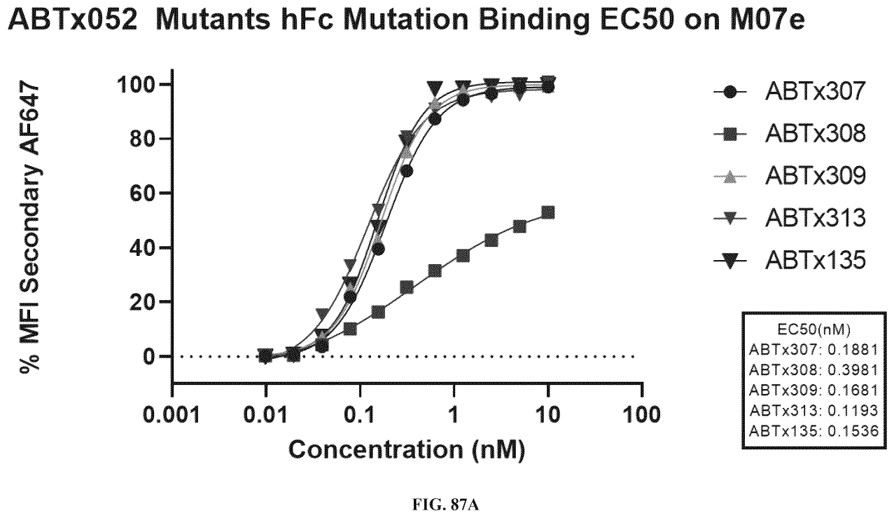

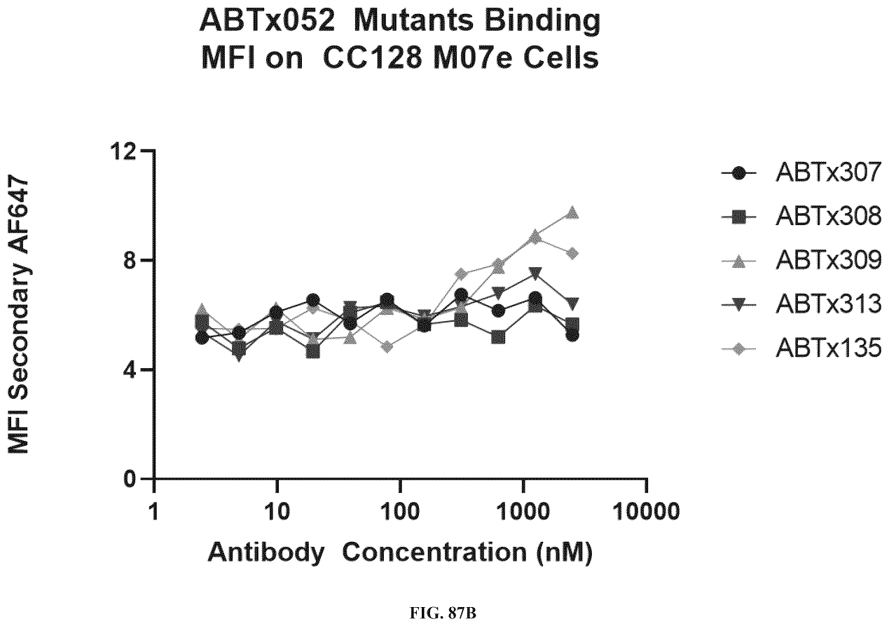

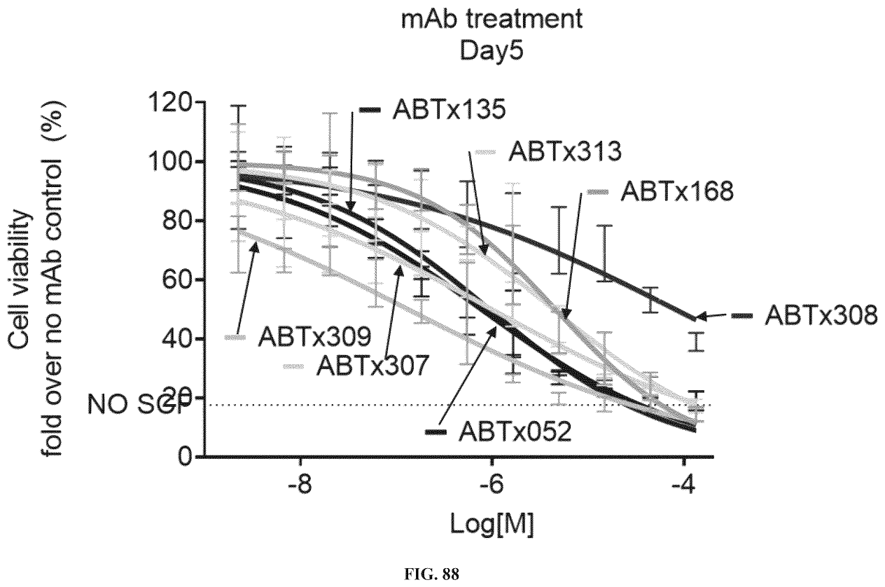

The ABTx307, ABTx308, ABTx309, and ABTx313 antibodies were expressed in an Expi 293 cell line, purified (FIGS. 78A-78D and 79), and characterized. The following properties of the antibodies were characterized: 1) Tm as determined by differential scanning fluorimetry (DSF) (FIGS. 80A and 80B and Table 76); 2) polyspecificity (FIGS. 81A and 81B); 3) KD for monovalent binding to CD117 (FIGS. 82A-82E and Table 77); 4) binding to CC128 and CD117 (FIGS. 83A-83E, 84, 85, 86A-86D, and Table 78); 5) EC50 on M07e cells (FIGS. 87A and 87B and Table 79); and 6) stem cell factor blocking (FIG. 88 and Table 80). The antibodies all had similar melting temperatures as ABTx135 (FIGS. 80A and 80B and Table 76). All of the antibodies had lower polyspecificity than ABTx135 (FIGS. 81A and 81B). ABTx308 had very low binding to CC128 (FIG. 85). Each of ABTx307, ABTx308, ABTx309, and ABTx313 had lower binding to CC128 than ABTx135 (FIGS. 86A-86D). ABTx052 and ABTx135 showed similar capacity to block SCF binding to human CD34+HSPCs at day 5 (FIG. 88). ABTx309 and ABTx307 showed IC50s for blocking SCF binding that were similar to ABTx135 and ABTx052, and ABTx313 had an IC50 for blocking SCF binding that was similar to ABTx168. Most of the monoclonal antibodies showed similar capacity for depleting human CD34+HSPCs (FIG. 88).

TABLE 76
Melting temperatures. The “control” sample
was provided with a kit used to measure melting
temperatures, and “C + L” indicates “control plus ligand.”

| Antibody ID | Tm1 | Tm2 |
|---|---|---|
| ABTx307 | 66.33 | 79.03 |
| ABTx308 | 66.48 | 76.67 |
| ABTx309 | 66.19 | 75.79 |
| ABTx313 | 66.33 | 79.03 |
| ABTx135 | 66.19 | 80.66 |
| control | 44.33 | 52.75 |
| C + L | 49.5 | |

TABLE 77
KD measurements.

| Antibody | KD | ka | kdis | Full | Full |
|---|---|---|---|---|---|
| ID | (M) | (1/Ms) | (1/s) | X\hat{ }2 | R\hat{ }2 |
| ABTx307 | 7.60E−09 | 1.57E+06 | 1.19E−02 | 2.0101 | 0.9843 |
| ABTx308 | 8.48E−09 | 1.95E+06 | 1.66E−02 | 1.0109 | 0.9863 |
| ABTx309 | 5.52E−09 | 1.47E+06 | 8.14E−03 | 2.0163 | 0.9864 |
| ABTx313 | 7.35E−09 | 1.62E+06 | 1.19E−02 | 1.8706 | 0.9846 |
| ABTx135 | 5.60E−09 | 1.63E+06 | 9.11E−03 | 1.3612 | 0.9907 |

TABLE 78
Binding to CC128.

| Antibody ID | R Max | kD | Ka | Koff | X\hat{ }2 | R\hat{ }2 |
|---|---|---|---|---|---|---|
| ABTx307 | 0.4941 | 5.11E−09 | 9.38E+06 | 4.80E−02 | 5.5661 | 0.9299 |
| ABTx308 | 0.2023 | 7.49E−09 | 8.96E+06 | 6.71E−02 | 0.9248 | 0.9248 |
| ABTx309 | 0.8522 | 4.67E−09 | 1.54E+07 | 7.20E−02 | 19.177 | 0.93 |
| ABTx313 | 0.383 | 9.61E−08 | 4.41E+06 | 4.24E−01 | 1.3928 | 0.9749 |
| ABTx135 | 0.5211 | 2.66E−08 | 1.59E+06 | 4.24E−02 | 1.7782 | 0.977 |

TABLE 79
EC50 on M07e cells expressing wild type CD117 and
M07e cells expressing a CC128 variant of CD117.

| Antibody ID | EC50 (nM) |
|---|---|
| ABTx307 | 0.1881 |
| ABTx308 | 0.3981 |
| ABTx309 | 0.1681 |
| ABTx313 | 0.1193 |
| ABTx135 | 0.1536 |

TABLE 80
IC50 (Inhibitory Concentration 50%) values for
the monoclonal antibodies on CD34+ HSPCs.

| | ABTx168 | ABTx309 | ABTx313 | ABTx307 | ABTx308 | ABTx052 | ABTx135 |
|---|---|---|---|---|---|---|---|
| IC50 | 4.56E−06 | 1.33E−07 | 4.37E−06 | 9.95E−07 | 8.05E−05 | 7.70E−07 | 9.27E−07 |
| R2 | 0.9387 | 0.8949 | 0.8826 | 0.895 | 0.6752 | 0.9423 | 0.92 |

The following methods were employed in the above examples.

### General HEK293T Mammalian Culture Conditions

Cells were cultured at 37° C. with 5% CO₂. HEK293T cells [CLBTx013, American Type Cell Culture Collection (ATCC)] were cultured in Dulbecco's modified Eagles medium plus Glutamax (10566-016, Thermo Fisher Scientific) with 10% (v/v) fetal bovine serum (A31606-02, Thermo Fisher Scientific). Cells were tested negative for mycoplasma after receipt from supplier.

mRNA Production for ABE Editors Used in CD34+ Cells

Editors were cloned into a plasmid encoding a dT7 promoter followed by a 5′UTR, Kozak sequence, ORF, and 3′UTR. The dT7 promoter carries an inactivating point mutation within the T7 promoter that prevents transcription from circular plasmid. This plasmid templated a PCR reaction (Q5 Hot Start 2X Master Mix), in which the forward primer corrected the SNP within the T7 promoter, and the reverse primer appended a 120A tail to the 3′ UTR. The resulting PCR product was purified on a Zymo Research 25 μg DCC column and used as mRNA template in the subsequent in vitro transcription. The NEB HiScribe High-Yield Kit was used as per the instruction manual but with full substitution of N1-methyl-pseudouridine for uridine and co-transcriptional capping with CleanCap AG (Trilink). Reaction cleanup was performed by lithium chloride precipitation. Primers used for amplification can be found in Table 26.

TABLE 81
Primers used for ABE8 T7 in vitro transcription reactions

| Name | Sequence |
|---|---|
| fwd_IVT | TCGAGCTCGGTACCTAATACGACTCAC (SEQ ID NO: 936) |
| rev_IVT | TTTTTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTTTTT |
| | TTTTTTTTTTTTCTTCCTACTCAGGCTTTATTCAAAGACCA (SEQ ID NO: 937) |

### CD34+ Cell Preparation

Mobilized peripheral blood was obtained and enriched for Human CD34+HSPCs (HemaCare, M001F-GCSF/MOZ-2). The CD34+ cells were thawed and put into X-VIVO 10 (Lonza) containing 1% Glutamax (Gibco), 100 ng/ml of TPO (Peprotech), SCF (Peprotech) and Flt-3 (Peprotech) at 48 hours prior to electroporation.

### Electroporation of CD34+ Cells

48 hours post thaw, the cells were spun down to remove the X-VIVO 10 media and washed in MaxCyte buffer (HyClone) with 0.1% HSA (Akron Biotechnologies). The cells were then resuspended in cold MaxCyte buffer at 1,250,000 cells per mL and split into multiple 20 μL aliquots. ABE mRNA and guide polynucleotides were then aliquoted as per the experimental conditions and raised to a total of 5 μL in MaxCyte buffer. The 20 μL of cells was the added into the 5 μL RNA mixture in groups of 3 and loaded into each chamber of an OC25x3 MaxCyte cuvette for electroporation. After receiving the charge, 25 μL was collected from the chambers and placed in the center of the wells in a 24-well untreated culture plate. The cells recovered for 20 minutes in an incubator (37° C., 5% CO₂). After the 20 minutes recovery, X-VIVO 10 (a hematopoietic cell medium) containing 1% Glutamax, 100 ng/ml of TPO, SCF and Flt-3 was added to the cells for a concentration of 1,000,000 cells per mL. The cells were then left to further recover in an incubator (37° C., 5% CO₂) for 48 hrs.

### Genomic DNA Extraction for CD34+ Cells

Following ABE electroporation (48 h later), an aliquot of cells was cultured in X-VIVO media (Lonza) containing 1% Glutamax (Gibco), 100 ng/ml of TPO (Peprotech), SCF (Peprotech) and Flt-3 (Peprotech). Following 48 h and 144 h post culturing, 100,000 cells were collected and spun down. 50 μL of Quick Extract (Lucigen) was added to the cell pellet and the cell mixture was transferred to a 96-well PCR plate (Bio-Rad). The lysate was heated for 15 minutes at 65° C. followed by 10 minutes at 98° C. The cell lysates were stored at −20° C.

### Polyspecificity

Some antibodies specifically or non-specifically bind more than one antigen, and this binding to multiple different antigens is referred to as “polyspecificity.” One potential reason for fast clearance of an antibody from the body of a subject is polyspecificity of the antibody. An ELISA assay for non-specific binding of antibodies to baculovirus particles (BVPs) can be used to determine polyspecificity of an antibody, and results from the assay are known to correlate with fast in vivo clearance of the antibodies. Accordingly, polyspecificity of antibodies of the disclosure was measured by using an ELISA assay to determine binding to BVP. In the assay, 1:1000, 1:10000, and 1:100000 dilutions of BVP in 50 mM sodium carbonate (pH 9.6) were indubated in wells surface-treated to bind the BVP for 16-24 hr at 4° C. Following the incubation, unbound BVP was removed from the wells and the wells were then incubated for 1 hr at room temperature with a blocking buffer (PBS containing 1% bovine serum albumin (BSA)) followed by three washes with PBST (phosphate-buffered saline with Tween 20). Antibodies to be characterized were then added to the wells as primary antibodies in 1:3 serial dilutions (˜1 μM undiluted antibody concentrations) and incubated for 1 hour at room temperature. The wells were again washed in PBST and anti-Human-IgG-HRP (horseradish peroxidase) was then added to the wells as a secondary antibody and incubated for 1 hr at room temperature. The wells were then washed 6 times using PBST followed by the addition of the HRP substrate 3,3′,5,5′-Tetramethylbenzidine (TMB). The reaction was stopped by adding 2M sulfuric acid to the wells and absorbance was then read at 450 nm. A “BVP score” was then calculated by normalizing absorbance to control wells that were not contacted with any primary antibody. For primary antibody serial diultion results, GraphPad Prism 9 was used to determine EC50.

### Other Embodiments

From the foregoing description, it will be apparent that variations and modifications may be made to the embodiments of the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed:

1. A method for hematopoietic stem cell transplantation in a subject, the method comprising:
(a) contacting an isolated hematopoietic stem cell or progenitor thereof with a guide polynucleotide and a base editor comprising a nucleic acid programmable DNA binding protein (napDNAbp) and an adenosine deaminase domain or a polynucleotide encoding the base editor, wherein the guide polynucleotide targets a nucleic acid molecule encoding a CD117 polypeptide, thereby generating an edited cell;
(b) administering the edited cell to the subject; and
(c) administering to the subject an antibody or antigen binding fragment thereof, wherein the antibody is selected from the group consisting of ABTx025, ABTx030, ABTx052, ABTx061, ABTx062, ABTx070, ABTx071, ABTx196, ABTx198, ABTx202, ABTx203, ABTx205, ABTx206, ABTx248, ABTx250, ABTx251, ABTx253, ABTx254, ABTx255, ABTx256, ABTx265, ABTx268, ABTx270, ABTx271, ABTx272, ABTx273, ABTx274, ABTx307, ABTx308, ABTx309, and ABTx313.

2. The method of claim 1, wherein in step (a) generating an edited cell comprises
i) introducing an alteration of a nucleobase in a codon encoding a serine at amino acid position 261, wherein the alteration of the nucleobase encoding the serine at amino acid position 261 results in the codon expressing a glycine,
ii) introducing an alteration of a nucleobase in a codon encoding a serine at amino acid position 251, and/or
iii) introducing an alteration of a nucleobase in a codon encoding an asparagine at amino acid position 260 and introducing an alteration of a nucleobase in a codon encoding a serine at amino acid position 261, or corresponding positions in another CD117 polypeptide, and generating an edited cell;

3. The method of claim 1, wherein the adenosine deaminase domain comprises a combination of alterations to TadA*7.10 selected from:
i) I76Y, V82T, Y123H, Y147R, F149Y, and Q154R; and
ii) I76Y, V82T, Y123H, Y147D, F149Y, Q154R, T166I, and D167N; wherein the adenosine deaminase domain has at least 85% sequence identity to TadA*7.10.

4. A method for treating a hemoglobinopathy in a subject, the method comprising:
(a) contacting an isolated hematopoietic stem cell or progenitor thereof with two or more guide polynucleotides and a base editor comprising a nucleic acid programmable DNA binding protein (napDNAbp) and an adenosine deaminase domain or a polynucleotide encoding the base editor, wherein one guide polynucleotide targets a nucleic acid molecule encoding a CD117 polypeptide and another guide polynucleotide targets the base editor to effect a deamination of a nucleobase of a hemoglobin subunit gamma 1 and/or 2 (HBG1/2) promoter, thereby generating an edited cell;
(b) administering the edited cell to the subject; and
(c) administering to the subject an antibody or antigen binding fragment thereof, wherein the antibody is selected from the group consisting of ABTx025, ABTx030, ABTx052, ABTx061, ABTx062, ABTx070, ABTx071, ABTx196, ABTx198, ABTx202, ABTx203, ABTx205, ABTx206, ABTx248, ABTx250, ABTx251, ABTx253, ABTx254, ABTx255, ABTx256, ABTx265, ABTx268, ABTx270, ABTx271, ABTx272, ABTx273, ABTx274, ABTx307, ABTx308, ABTx309, and ABTx313.

5. The method of claim 4, wherein in step (a),
1. one guide polynucleotide targets a nucleic acid molecule encoding a CD117 polypeptide, thereby
i) introducing an alteration of a nucleobase in a codon encoding a serine at amino acid position 261, wherein the alteration of the nucleobase encoding the serine at amino acid position 261 results in the codon expressing a glycine,
ii) introducing an alteration of a nucleobase in a codon encoding a serine at amino acid position 251, and/or
iii) introducing an alteration of a nucleobase in a codon encoding an asparagine at amino acid position 260 and introducing an alteration of a nucleobase in a codon encoding a serine at amino acid position 261 or corresponding positions in another CD117 polypeptide; and
2) another guide polynucleotide targets the base editor to effect an alteration to a beta globin polynucleotide (HBB) that results in expression of a beta globin polypeptide having an alanine at position 6 (Hb G-Makassar), thereby generating an edited cell;
(b) administering the edited cell to the subject; and
(c) administering to the subject an antibody or antigen binding fragment thereof, antibody drug conjugate, or a chimeric antigen receptor T (CAR-T) cell, each of which selectively binds a wild type CD117 polypeptide.

6. A method of altering a nucleobase of a CD117 polynucleotide, the method comprising:
contacting the CD117 polynucleotide with a base editor polypeptide comprising a nucleic acid programmable DNA binding protein (napDNAbp) and an adenosine deaminase domain, and a guide polynucleotide that targets said base editor to
i) effect an alteration of a nucleobase in a codon encoding a serine at amino acid position 261, wherein the alteration of the nucleobase encoding the serine at amino acid position 261 results in the codon expressing a glycine,
ii) effect an alteration of a nucleobase in a codon encoding a serine at amino acid position 251, and/or
iii) effect an alteration of a nucleobase in a codon encoding an asparagine at amino acid position 260 and effect an alteration of a nucleobase in a codon encoding a serine at amino acid position 261, or corresponding positions in another CD117 polypeptide, thereby altering the nucleobase of the CD117 polynucleotide.

7. The method of claim 6, wherein the adenosine deaminase domain comprises a combination of alterations to TadA*7.10 selected from:
a) I76Y, V82T, Y123H, Y147R, F149Y, and Q154R; and
b) I76Y, V82T, Y123H, Y147D, F149Y, Q154R, T166I, and D167N; wherein the adenosine deaminase domain has at least 85% sequence identity to TadA*7.10, and a guide polynucleotide that targets the base editor to effect an alteration of a nucleobase in a polynucleotide encoding a CD117 polypeptide, thereby altering the nucleobase of the CD117 polynucleotide.

8. A cell produced by the method of claim 1.

9. A pharmaceutical composition comprising an effective amount of the cell of claim 8.

10. A base editor system comprising a base editor comprising a nucleic acid programmable DNA binding protein (napDNAbp) and an adenosine deaminase domain or a polynucleotide encoding the base editor, and a guide polynucleotide that targets said base editor to
i) effect an alteration of a nucleobase in a codon encoding a serine at amino acid position 261, wherein the alteration of the nucleobase encoding the serine at amino acid position 261 results in the codon expressing a glycine,
ii) effect an alteration of a nucleobase in a codon encoding a serine at amino acid position 251, and/or
iii) effect an alteration of a nucleobase in a codon encoding an asparagine at amino acid position 260 and effect an alteration of a nucleobase in a codon encoding a serine at amino acid position 261, or corresponding positions in another CD117 polypeptide, thereby altering the nucleobase of the CD117 polynucleotide.

11. The base editor system of claim 10, wherein the adenosine deaminase domain comprises a combination of alterations to TadA*7.10, wherein the combinations are selected from:
a) I76Y, V82T, Y123H, Y147R, F149Y, and Q154R; and
b) I76Y, V82T, Y123H, Y147D, F149Y, Q154R, T166I, and D167N; wherein the guide polynucleotide targets the base editor to effect an alteration of a nucleobase of a CD117 polynucleotide, wherein the adenosine deaminase domain has at least 85% sequence identity to TadA*7.10.

12. The base editor system of claim 10, wherein the guide polynucleotide comprises a polynucleotide sequence selected from the group consisting of: AUAAUAGCUGGCAUCACGGU (SEQ ID NO: 693; CC200);
(SEQ ID NO: 694; gRNA889)
CCACUAGCUUUCCAAACGGU;
(SEQ ID NO: 695; gRNA908)
GCUGAACUGAUAGUCAACGU;
(SEQ ID NO: 696; gRNA918)
UUUGACAAAGCCCGGAUCAG;
(SEQ ID NO: 697; gRNA923)
UGAAAGUGAGGCCAGGUACU;
(SEQ ID NO: 698; gRNA928)
AAACAGUCAGGUGAGUGAAU;
(SEQ ID NO: 699; gRNA929)
AACUACAGGAGAAAUAUAAU;
and
(SEQ ID NO: 700; gRNA944)
GAUUAAAAGGCACCGAAGGA.

13. A polynucleotide encoding the base editor system of claim 10.

14. A guide polynucleotide comprising a spacer sequence selected from the group consisting of:
(SEQ ID NO: 693; gRNA931)
AUAAUAGCUGGCAUCACGGU;
(SEQ ID NO: 694; gRNA889)
CCACUAGCUUUCCAAACGGU;
(SEQ ID NO: 695; gRNA908)
GCUGAACUGAUAGUCAACGU;
(SEQ ID NO: 696; gRNA918)
UUUGACAAAGCCCGGAUCAG;
(SEQ ID NO: 697; gRNA923)
UGAAAGUGAGGCCAGGUACU;
(SEQ ID NO: 698; gRNA928)
AAACAGUCAGGUGAGUGAAU;
(SEQ ID NO: 699; gRNA929)
AACUACAGGAGAAAUAUAAU;
and
(SEQ ID NO: 700; gRNA944)
GAUUAAAAGGCACCGAAGGA.

15. A kit comprising base editor system of claim 10.

16. An anti-CD117 antibody or antigen-binding portion thereof comprising one or more complementarity determining regions (CDRs) which comprise or consist of heavy chain variable region (VH) CDRs and/or light chain variable region (VL) CDRs selected from the following:
A)
VL CDR1:
(SEQ ID NO: 394)
QSVSSSY;
VL CDR2:
GAS;
VL CDR3:
(SEQ ID NO: 395)
QQYGTSLT;
VH CDR1:
(SEQ ID NO: 391)
GFTFDDYA;
VH CDR2:
(SEQ ID NO: 392)
ISWNSGTI;
VH CDR3:
(SEQ ID NO: 393)
AKDSPPGYCSSASCYGAFDI (ABTx025);
B)
VL CDR1:
(SEQ ID NO: 409)
QSISSY;
VL CDR2:
AAS;
VL CDR3:
(SEQ ID NO: 410)
QQSYSTPLT;
VH CDR1:
(SEQ ID NO: 406)
GFTFSSYS;
VH CDR2:
(SEQ ID NO: 407)
IGTISSYI;
VH CDR3:
(SEQ ID NO: 408)
ARDYYGGLFDY (ABTx030);
C)
VL CDR1:
(SEQ ID NO: 439)
QSVSSSY;
VL CDR2:
GAS;
VL CDR3:
(SEQ ID NO: 440)
QQYGSSPLT;
VH CDR1:
(SEQ ID NO: 436)
GFTFDDYA;
VH CDR2:
(SEQ ID NO: 437)
ISWNSGSI;
VH CDR3:
(SEQ ID NO: 438)
AKDTPLGYCSTTSCYGAFDI (ABTx061);
D)
VL CDR1:
(SEQ ID NO: 454)
QSISSY;
VL CDR2:
AAS;
VL CDR3:
(SEQ ID NO: 455)
QQSYSTPFT;
VH CDR1:
(SEQ ID NO: 451)
GFTFDDYA;
VH CDR2:
(SEQ ID NO: 452)
ISWNSGTI;
VH CDR3:
(SEQ ID NO: 453)
AKDSPPGYCSSASCYGAFDI (ABTx062);
E)
VL CDR1:
(SEQ ID NO: 469)
QGISSY;
VL CDR2:
AAS;
VL CDR3:
(SEQ ID NO: 470)
QQSYSTPIT;
VH CDR1:
(SEQ ID NO: 466)
GFTFDDYG;
VH CDR2:
(SEQ ID NO: 467)
INWNGGST;
VH CDR3:
(SEQ ID NO: 468)
ARESWDGSGIYYMDV (ABTx070);
F)
VL CDR1:
(SEQ ID NO: 484)
QGISSY;
VL CDR2:
AAS;
VL CDR3:
(SEQ ID NO: 485)
QQLNSYPYT;
VH CDR1:
(SEQ ID NO:481)
GFTFDDYG;
VH CDR2:
(SEQ ID NO: 482)
INWNGGST;
VH CDR3:
(SEQ ID NO: 483)
ARESWNYEGYYYMDV (ABTx071);
G)
VL CDR1:
(SEQ ID NO: 424)
QGISSA;
VL CDR2:
DAS;
VL CDR3:
(SEQ ID NO: 425)
QQFNSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 1012)
ARHGRGYDGYDGAFDI;
H)
VL CDR1:
(SEQ ID NO: 424)
QGISSA;
VL CDR2:
DAS;
VL CDR3:
(SEQ ID NO: 425)
QQFNSYPLT;
VH CDR1:
(SEQ ID NO: 421)
GYRFTSYW;
VH CDR2:
(SEQ ID NO: 972)
IYPGDSDTK;
VH CDR3:
(SEQ ID NO: 948)
ARHGRGYDGYEGAFDI;
I)
VL CDR1:
(SEQ ID NO: 424)
QGISSA;
VL CDR2:
DAS;
VL CDR3:
(SEQ ID NO: 954)
QQFYSYPLT;
VH CDR1:
(SEQ ID NO: 421)
GYRFTSYW;
VH CDR2:
(SEQ ID NO: 958)
TYPGDSDTR;
VH CDR3:
(SEQ ID NO: 948)
ARHGRGYDGYEGAFDI;
J)
VL CDR1:
(SEQ ID NO: 424)
QGISSA;
VL CDR2:
DAS;
VL CDR3:
(SEQ ID NO: 1020)
QQFSSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 948)
ARHGRGYDGYEGAFDI;
K)
VL CDR1:
(SEQ ID NO: 424)
QGISSA;
VL CDR2:
DAS;
VL CDR3:
(SEQ ID NO: 1023)
QQTNSHPLT;
VH CDR1:
(SEQ ID NO: 421)
GYRFTSYW;
VH CDR2:
(SEQ ID NO: 972)
IYPGDSDTK;
VH CDR3:
(SEQ ID NO: 1012)
ARHGRGYDGYDGAFDI;
L)
VL CDR1:
(SEQ ID NO: 424)
QGISSA;
VL CDR2:
DAS;
VL CDR3:
(SEQ ID NO: 1025)
QQVRSYPLT;
VH CDR1:
(SEQ ID NO: 421)
GYRFTSYW;
VH CDR2:
(SEQ ID NO: 972)
IYPGDSDTK;
VH CDR3:
(SEQ ID NO: 948)
ARHGRGYDGYEGAFDI;
M)
VL CDR1:
(SEQ ID NO: 424)
QGISSA;
VL CDR2:
DAS;
VL CDR3:
(SEQ ID NO: 1029)
QQTYSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 1027)
ARHGRGYDAYEGAFDI;
N)
VL CDR1:
(SEQ ID NO: 424)
QGISSA;
VL CDR2:
DAS;
VL CDR3:
(SEQ ID NO: 1036)
QQTRSYPLT;
VH CDR1:
(SEQ ID NO: 421)
GYRFTSYW;
VH CDR2:
(SEQ ID NO: 972)
IYPGDSDTK;
VH CDR3:
(SEQ ID NO: 1012)
ARHGRGYDGYDGAFDI;
O)
VL CDR1:
(SEQ ID NO: 424)
QGISSA;
VL CDR2:
DAS;
VL CDR3:
(SEQ ID NO: 952)
QQVNSYPLT;
VH CDR1:
(SEQ ID NO: 421)
GYRFTSYW;
VH CDR2:
(SEQ ID NO: 972)
IYPGDSDTK;
VH CDR3:
(SEQ ID NO: 948)
ARHGRGYDGYEGAFDI;
P)
VL CDR1:
(SEQ ID NO: 424)
QGISSA;
VL CDR2:
DAS;
VL CDR3:
(SEQ ID NO: 1025)
QQVRSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 1027)
ARHGRGYDAYEGAFDI;
Q)
VL CDR1:
(SEQ ID NO: 424)
QGISSA;
VL CDR2:
DAS;
VL CDR3:
(SEQ ID NO: 1044)
QQTNSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 1032)
ARHGRGYDAYDGAFDI;
R)
VL CDR1:
(SEQ ID NO: 424)
QGISSA;
VL CDR2:
DAS;
VL CDR3:
(SEQ ID NO: 1025)
QQVRSYPLT;
VH CDR1:
(SEQ ID NO: 421)
GYRFTSYW;
VH CDR2:
(SEQ ID NO: 972)
IYPGDSDTK;
VH CDR3:
(SEQ ID NO: 1012)
ARHGRGYDGYDGAFDI;
S)
VL CDR1:
(SEQ ID NO: 424)
QGISSA;
VL CDR2:
DAS;
VL CDR3:
(SEQ ID NO: 953)
QQFRSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 948)
ARHGRGYDGYEGAFDI;
T)
VL CDR1:
(SEQ ID NO: 424)
QGISSA;
VL CDR2:
DAS;
VL CDR3:
(SEQ ID NO: 1044)
QQTNSYPLT;
VH CDR1:
(SEQ ID NO: 421)
GYRFTSYW;
VH CDR2:
(SEQ ID NO: 972)
TYPGDSDTK;
VH CDR3:
(SEQ ID NO: 948)
ARHGRGYDGYEGAFDI;
U)
VL CDR1:
(SEQ ID NO: 394)
QSVSSSY;
VL CDR2:
GAS;
VL CDR3:
(SEQ ID NO: 1074)
QQSETCLT;
VH CDR1:
(SEQ ID NO: 391)
GFTFDDYA
VH CDR2:
(SEQ ID NO: 1070)
ISWNSGTIG;
VH CDR3:
(SEQ ID NO: 393)
AKDSPPGYCSSASCYGAFDI;
V)
VL CDR1:
(SEQ ID NO: 394)
QSVSSSY;
VL CDR2:
GAS;
VL CDR3:
(SEQ ID NO: 1074)
QQDSLGLT;
VH CDR1:
(SEQ ID NO: 391)
GFTFDDYA;
VH CDR2:
(SEQ ID NO: 1070)
ISWNSGTIG;
VH CDR3:
(SEQ ID NO: 1090)
AKDSPPGYCASASCYGAFDI;
W)
VL CDR1:
(SEQ ID NO: 394)
QSVSSSY;
VL CDR2:
GSS;
VL CDR3:
(SEQ ID NO: 1084)
QQYNFWPYT;
VH CDR1:
(SEQ ID NO: 391)
GFTFDDYA;
VH CDR2:
(SEQ ID NO: 1070)
ISWNSGTIG;
VH CDR3:
(SEQ ID NO: 393)
AKDSPPGYCSSASCYGAFDI;
X)
VL CDR1:
(SEQ ID NO: 469)
QGISSY;
VL CDR2:
AAS;
VL CDR3:
(SEQ ID NO: 1097)
QQSYSTPYT;
VH CDR1:
(SEQ ID NO: 391)
GFTFDDYA;
VH CDR2:
(SEQ ID NO: 1070)
ISWNSGTIG;
VH CDR3:
(SEQ ID NO: 1094)
AKDWPSGFCSSAYCYGAFDI;
Y)
VL CDR1:
(SEQ ID NO: 469)
QGISSY;
VL CDR2:
AAS;
VL CDR3:
(SEQ ID NO: 1097)
QQSYSTPYT;
VH CDR1:
(SEQ ID NO: 1071)
GFTFDDYA;
VH CDR2:
(SEQ ID NO: 1070)
ISWNSGTIG;
VH CDR3:
(SEQ ID NO: 393)
AKDSPPGYCSSASCYGAFDI;
Z)
VL CDR1:
(SEQ ID NO: 424)
QGISSA;
VL CDR2:
DAS;
VL CDR3:
(SEQ ID NO: 425)
QQFNSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 1012)
ARHGRGYDGYDGAFDI;
AA)
VL CDR1:
(SEQ ID NO: 424)
QGISSA;
VL CDR2:
DAS;
VL CDR3:
(SEQ ID NO: 1020)
QQFSSYPLT;
VH CDR1:
(SEQ ID NO: 421)
GYRFTSYW;
VH CDR2:
(SEQ ID NO: 972)
TYPGDSDTK;
VH CDR3:
(SEQ ID NO: 1032)
ARHGRGYDAYDGAFDI;
AB)
VL CDR1:
(SEQ ID NO: 424)
QGISSA;
VL CDR2:
DAS;
VL CDR3:
(SEQ ID NO: 1044)
QQTNSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 1032)
ARHGRGYDAYDGAFDI;
and
AC)
VL CDR1:
(SEQ ID NO: 424)
QGISSA;
VL CDR2:
DAS;
VL CDR3:
(SEQ ID NO: 425)
QQFSSYPLT;
VH CDR1:
(SEQ ID NO: 421)
GYRFTSYW;
VH CDR2:
(SEQ ID NO: 958)
IYPGDSDTR;
VH CDR3:
(SEQ ID NO: 1012)
ARHGRGYDGYDGAFDI.

17. An isolated polynucleotide that encodes the antibody of claim 16.

18. An anti-CD117 antibody or antigen-binding portion thereof comprising complementarity determining regions (CDRs) that comprise or consist of the following heavy chain variable region (VH) CDR and light chain variable region (VL) CDR amino acid sequences:
VH CDR1: GX₁X₂FX₃X₄YX₅, wherein
X₁ is F or Y,
X₂ is R or T,
X₃ is D, S, or T,
X₄ is D or S, and
X₅ is A, G, S, or W;
VH CDR2 is IX₆X₇X₈X₉X₁₀X₁₁X₁₂X₁₃, wherein
X₆ is G, N, S, or Y,
X₇ is P, T, or W,
X₈ is G, I, or N,
X₉ is D, G, or S,
X₁₀ is Gor S,
X₁₁ is D, S, T, or Y,
X₁₂ is I or T, and
X₁₃ is G, K, R, or Y;
VH CDR3 is selected from the group consisting of ARHGRGYDX₁₄ YDGAFDI (SEQ ID NO: 1105), ARDYYGGLFDY (SEQ ID NO: 1106), ARESWX₁₅X₁₆X₁₇GX₁₈ YYMDV (SEQ ID NO: 1107), and AKDX₁₉PX₂₀GX₂₁CX₂₂X₂₃X₂₄X₂₅CYGAFDI (SEQ ID NO: 1108), wherein
X₁₄ is A or G,
X₁₅ is D or N,
X₁₆ is Gor Y,
X₁₇ is E or S,
X₁₈ is I or Y,
X₁₉ is S, T, or W,
X₂₀ is L, P, or S,
X₂₁ is F or Y,
X₂₂ is A or S,
X₂₃ is S or T,
X₂₄ is A or T, and
X₂₅ is S or Y;
VL CDR1 is QSX₂₆SSX₂₇ (SEQ ID NO: 1109) or QSVSSSY (SEQ ID NO: 1110), wherein
X₂₆ is Gor S, and
X₂₇ is A or Y;
VL CDR2: X₂₈X₂₉S, wherein,
X₂₈ is A, D, or G, and
X₂₉ is A or S; and
VL CDR3 is QQX₃₀X₃₁X₃₂X₃₃PX₃₄T (SEQ ID NO: 1111) or QQX₃₅X₃₆X₃₇X₃₈LT (SEQ ID NO: 1112), wherein
X₃₀ is F, L, S, T, or Y,
X₃₁ G, N, S, or Y,
X₃₂ is S or F,
X₃₃ S, T, W, or Y,
X₃₄ F, I, L, or Y,
X₃₅ is D, S, or Y,
X₃₆ is E, G, or S,
X₃₇ is L or T, and
X₃₈ is C, G, or S;
and wherein the anti-CD117 antibody comprises at least one amino acid alteration relative to the amino acid sequence of ABTx052.

19. A method for hematopoietic stem cell transplantation in a subject, the method comprising:
(a) administering a hematopoietic stem cell or progenitor thereof to the subject, wherein the hematopoietic stem cell or progenitor thereof expresses a CD117 variant comprising an S261G amino acid alteration, or a CD117 variant comprising Y259C and N260D amino acid alterations; and
(b) administering to the subject an antibody or antigen binding fragment thereof that selectively binds a wild type CD117 polypeptide.

20. The method of claim 19, wherein the hematopoietic stem cell or progenitor thereof:
i) either expresses a CD117 variant comprising an S261G amino acid alteration or a CD117 variant comprising Y259C and N260D amino acid alterations, and
ii) comprises a nucleobase alteration to the HBG1/2 promoter that effects an increase in gamma globin expression and/or expresses an HBB polypeptide comprising an alanine at position 6.

21. A hematopoietic stem cell or progenitor thereof expressing a CD117 polypeptide comprising
i) an S261G alteration,
ii) alterations at amino acid positions 260 and 261, and/or
ii) an alteration at amino acid position 251, relative to the following amino acid sequence,
wherein the CD117 polypeptide has at least 85% sequence identity to the following amino acid sequence:
Wild Type CD117
(SEQ ID NO: 499)
MRGARGAWDFLCVLLLLLRVQTGSSQPSVSPGEPSPPSIHPGKSDLIVRVGDEIRLLCTDPGFV
KWTFEILDETNENKQNEWITEKAEATNTGKYTCTNKHGLSNSIYVFVRDPAKLFLVDRSLYGKE
DNDTLVRCPLTDPEVTNYSLKGCQGKPLPKDLRFIPDPKAGIMIKSVKRAYHRLCLHCSVDQEG
KSVLSEKFILKVRPAFKAVPVVSVSKASYLLREGEEFTVTCTIKDVSSSVYSTWKRENSQTKLQ
EKYNSWHHGDFNYERQATLTISSARVNDSGVFMCYANNTFGSANVTTTLEVVDKGFINIFPMIN
TTVFVNDGENVDLIVEYEAFPKPEHQQWIYMNRTFTDKWEDYPKSENESNIRYVSELHLTRLKG
TEGGTYTFLVSNSDVNAAIAFNVYVNTKPEILTYDRLVNGMLQCVAAGFPEPTIDWYFCPGTEQ
RCSASVLPVDVQTLNSSGPPFGKLVVQSSIDSSAFKHNGTVECKAYNDVGKTSAYFNFAFKGNN
KEQIHPHTLFTPLLIGFVIVAGMMCIIVMILTYKYLQKPMYEVQWKVVEEINGNNYVYIDPTQL
PYDHKWEFPRNRLSFGKTLGAGAFGKVVEATAYGLIKSDAAMTVAVKMLKPSAHLTEREALMSE
LKVLSYLGNHMNIVNLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTPAIMEDDELALDLED
LLSFSYQVAKGMAFLASKNCIHRDLAARNILLTHGRITKICDFGLARDIKNDSNYVVKGNARLP
VKWMAPESIFNCVYTFESDVWSYGIFLWELFSLGSSPYPGMPVDSKFYKMIKEGFRMLSPEHAP
AEMYDIMKTCWDADPLKRPTFKQIVQLIEKQISESTNHIYSNLANCSPNRQKPVVDHSVRINSV
GSTASSSQPLLVHDDV

22. The hematopoietic stem cell or progenitor thereof expressing a CD117 polypeptide, the CD117 polypeptide comprising a sequence comprising 10, 20, 30, or 40 consecutive amino acids, wherein the sequence of consecutive amino acids comprises amino acid position 260, amino acid positions 261, and/or amino acid position 251 relative to the following amino acid sequence, wherein:
i) an amino acid corresponding to amino acid position 261 is substituted with a glycine,
ii) the amino acids corresponding to amino acid positions 260 and 261 are altered, and/or
iii) an amino acid corresponding to amino acid position 251 is altered relative to the following amino acid sequence in the sequence of consecutive amino acids, wherein the sequence of consecutive amino acids has at least 85% sequence identity to a fragment of the following amino acid sequence that has the same length as the sequence of consecutive amino acids, and wherein the CD117 polypeptide is capable of binding a stem cell factor (SCF) polypeptide:
Wild Type CD117
(SEQ ID NO: 499)
MRGARGAWDFLCVLLLLLRVQTGSSQPSVSPGEPSPPSIHPGKSDLIVRVGDEIRLLCTDPGFV
KWTFEILDETNENKQNEWITEKAEATNTGKYTCTNKHGLSNSIYVFVRDPAKLFLVDRSLYGKE
DNDTLVRCPLTDPEVTNYSLKGCQGKPLPKDLRFIPDPKAGIMIKSVKRAYHRLCLHCSVDQEG
KSVLSEKFILKVRPAFKAVPVVSVSKASYLLREGEEFTVTCTIKDVSSSVYSTWKRENSQTKLQ
EKYNSWHHGDFNYERQATLTISSARVNDSGVFMCYANNTFGSANVTTTLEVVDKGFINIFPMIN
TTVFVNDGENVDLIVEYEAFPKPEHQQWIYMNRTFTDKWEDYPKSENESNIRYVSELHLTRLKG
TEGGTYTFLVSNSDVNAAIAFNVYVNTKPEILTYDRLVNGMLQCVAAGFPEPTIDWYFCPGTEQ
RCSASVLPVDVQTLNSSGPPFGKLVVQSSIDSSAFKHNGTVECKAYNDVGKTSAYFNFAFKGNN
KEQIHPHTLFTPLLIGFVIVAGMMCIIVMILTYKYLQKPMYEVQWKVVEEINGNNYVYIDPTQL
PYDHKWEFPRNRLSFGKTLGAGAFGKVVEATAYGLIKSDAAMTVAVKMLKPSAHLTEREALMSE
LKVLSYLGNHMNIVNLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTPAIMEDDELALDLED
LLSFSYQVAKGMAFLASKNCIHRDLAARNILLTHGRITKICDFGLARDIKNDSNYVVKGNARLP
VKWMAPESIFNCVYTFESDVWSYGIFLWELFSLGSSPYPGMPVDSKFYKMIKEGFRMLSPEHAP
AEMYDIMKTCWDADPLKRPTFKQIVQLIEKQISESTNHIYSNLANCSPNRQKPVVDHSVRINSV
GSTASSSQPLLVHDDV
